(19) United States
(12) Patent Application Publication
(10) Pub. No.: US 2025/0241922 A1
(43) Pub. Date: Jul. 31, 2025

(54) ANTI-INFLAMMATORY COUPLING COMPOUND DRUG, AND PREPARATION METHOD THEREFOR AND USE THEREOF

(71) Applicant: COVAL BIOPHARMA (SHANGHAI) CO., LTD., Shanghai (CN)

(72) Inventors: Lichun FENG, Shanghai (CN); Weijiang ZHANG, Redwood City, CA (US); Guolong WU, Shanghai (CN); Hao ZHANG, Foster City, CA (US); Dafeng LI, Shanghai (CN)

(21) Appl. No.: 19/183,821

(22) Filed: Apr. 19, 2025

Related U.S. Application Data
(63) Continuation of application No. 18/413,842, filed on Jan. 16, 2024, now Pat. No. 12,290,519.
Application No. 18/413,842 is a continuation of application No. PCT/CN2022/105503, filed on Jul. 13, 2022.

(30) Foreign Application Priority Data
Jul. 20, 2021 (CN) 202110834326.4

(51) Int. Cl. A61K 31/519; A61P 29/00

(52) U.S. Cl. CPC A61K 31/519; A61P 29/00

(57) ABSTRACT

An anti-inflammatory drug compound, and a preparation method therefor and the use thereof. The structural formula of the compound is A-Y—B, wherein A is a group after dehydrogenation of an amine compound having JAK inhibitory activity, Y is a direct connection or —(CH2)-O— or, and B is a group formed by means of dehydroxylation of a carboxy-containing carboxylic acid compound B1, or a group formed by means of dehydrogenation of a hydroxy-containing compound B2. The compound has the special effects of having a strong transdermal property, controlled drug release, high efficacy, etc.

### CROSS-REFERENCE TO RELATED APPLICATIONS

This application is a continuation of U.S. patent application Ser. No. 18/413,842 filed Jan. 16, 2024, which is a continuation of International Patent Application No. PCT/CN2022/105503 filed Jul. 13, 2022, which claims priority to Chinese Patent Application No. 202110834326.4 filed Jul. 20, 2021. The entire contents of each of the aforementioned applications is incorporated herein by reference.

### TECHNICAL FIELD

The present invention relates to an anti-inflammatory drug suitable for external use, and more particularly, to a compound drug synthesized through coupling and use thereof.

### BACKGROUND ART

JAK-STAT signaling pathway is a cytokine-stimulated signal transduction pathway, involved in cell proliferation, differentiation, apoptosis and immune regulation, and other important biological processes. Many cytokines and growth factors signal through the JAK-STAT signaling pathway, including IL (interleukins), GM-CSF (granulocyte/macrophage colony-stimulating factor), GH (growth hormone), EGF (epidermal growth factor), PDGF (platelet-derived factor), and IFN (interferons), among others.

The JAK-STAT signaling pathway consists of three components, a receptor, a tyrosine kinase (JAK), and a transcription factor (STAT). Upon binding of the ligand to the tyrosine kinase-related receptor, the JAK bound thereto is activated. The activated JAK further activates the corresponding STAT protein. The activated STAT protein enters the nucleus to bind to a target gene and regulates the gene transcription.

The JAK family includes JAK1, JAK2, JAK3, and TYK2. These kinases control seven different STATs, namely STAT1, STAT2, STAT3, STAT4, STAT5A, STAT5B, and STAT6. It is precisely by controlling these STATs that cytokines and growth factors can control cell proliferation, differentiation, apoptosis, and immune regulation. Many diseases arise from JAK and STAT mutations. By modulating and selectively inhibiting certain JAK, these diseases can be effectively treated.

Although JAK-STAT is a relatively simple signaling pathway, it is involved in many cellular functions. There are dozens of JAK inhibitors currently on the market and under research. The vast majority of JAK inhibitor programs seek to maximize efficacy and minimize side effects by selectively inhibiting one of the JAK-STAT signaling. However, systemic administration is often unable to circumvent the contradiction between efficacy and side effects. Topical administration of JAK inhibitors may enhance efficacy while reducing systemic side effects. Many of the JAK inhibitor programs under research also have organ selectivity as the ultimate goal to maximize efficacy/risk.

It is also an object of the present invention to maximize the therapeutic effect of dermal administration and minimize systemic toxicity by topical (dermal) administration in combination with optimization of the structure of the compound. Many skin diseases, including psoriasis, vitiligo, alopecia areata, etc. have a clear understanding of their causes and mechanisms of disease treatment. The regulatory and control mechanisms of various JAK-STATs by JAK inhibitors currently on the market and under research have also been very clear. The purpose of selective dermal administration can also be achieved by modifying the chemical structure of the currently known JAK inhibitors to allow them to penetrate more through the protective layer of the dermal. Compared with the development of a skin-selective drug delivery formulation of a completely new compound, the product with a known compound with an optimized structure has a relatively short development cycle, low risk of efficacy failure, low possibility of unknown toxicity and low development cost.

The optimization of a compound structure by increasing compound transdermal penetration can change the physical and chemical properties of the compound by adding nonfunctional groups to the known compound structure, allowing it to penetrate more through the protective layer of the dermal.

### SUMMARY OF THE INVENTION

The present invention is directed to the technical problem that existing JAK inhibitors also require high doses or have side effects, and seeks to optimize the structure of the known JAK compounds so that they can effectively achieve skin-selective administration, thereby increasing the effectiveness for skin diseases and reducing systemic dose and side effects. More specifically, structural optimization and skin-selective administration of known JAK inhibitors are achieved by coupling with other small molecule compounds.

Specifically, the present invention provides the following technical solutions.
1. Depending on the structure of the different JAK inhibitor compounds, specific linkers and coupling small molecules are chosen to allow more final compound (prodrug) to penetrate the protective layer of the dermal.
2. The chemical bond between the linker and the JAK inhibitor is unstable in the human skin, leading to hydrolysis and release of effective ingredients, known as JAK inhibitors.
3. The linkage between the linker and the coupled small molecule is also unstable, leading to the release of the coupled small molecule.
4. The linker is known per se as a simple chemical structure without toxicity.
5. The chemical structure of coupled small molecules is clear, and the pharmacology and toxicology are known.

Specifically, the present invention provides the following technical solutions.

In an aspect, the present invention provides an anti-inflammatory compound, or a stereoisomer, tautomer, nitrogen oxide, metabolite, prodrug, pharmaceutically acceptable salt, or solvate thereof, having a structure shown in general formula (I):

A-Y—B (I)
wherein, A is a group after dehydrogenation of an amine compound having JAK inhibitory activity;
Y is a direct connection or —(CH₂)—O— or —(CH₂)—;
B is a group formed by means of dehydroxylation of a carboxy-containing carboxylic acid compound B₁, or a group formed by means of dehydrogenation of a hydroxy-containing compound B₂; and wherein, in the case where the carboxylic acid B₁ is dehydroxylated to form a group, the Y group is a direct connection or —(CH₂)—O—; in the case where the hydroxy-containing compound B₂ is dehydrogenated to form a group (i.e., B is B₂), the Y group is —(CH₂)—.

In a specific embodiment, the anti-inflammatory compound, or the stereoisomer, tautomer, nitrogen oxide, metabolite, prodrug, pharmaceutically acceptable salt, or solvate thereof described above, having a structure shown in general formulas (II) or (IIa):

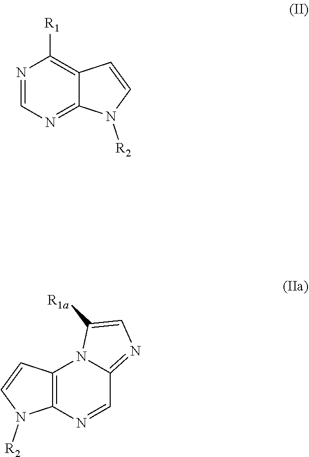

wherein R₁ is selected from pyrazolyl or pyrrolyl unsubstituted or substituted with Rₐ; or —N(CH₃)—Cy; R₁ₐ represents a pyrrole ring substituted by a halogen-substituted C₁-C₆ alkylaminoacyl group and/or by a C₁-C₆ alkyl group;
C_{y} is a five- or six-membered carbocyclic ring or a five- or six-membered nitrogen-containing heterocyclic ring unsubstituted or substituted by R_{b}; Rₐ and R_{b} are each independently groups containing at least one or two groups selected from a group consisting of an acyl group, a sulfonyl group, a cyano group, an amino group or a C₁-C₆ alkyl-substituted amino group, and a four-, five-, or six-membered nitrogen-containing heterocyclic group, or the nitrogen-containing heterocyclic group substituted with C₁-C₆ alkyl; preferably Rₐ and R_{b} are each independently groups which consist of one group of acyl or sulfonyl and at least one group selected from a group consisting of cyano, amino or C₁-C₆ alkyl substituted amino, and a four-, five-, or six-membered nitrogen-containing heterocyclyl, or the nitrogen-containing heterocyclyl substituted with C₁-C₆ alkyl, wherein the C₁-C₆ alkyl is substitutable by halogen; That is, Rₐ and R_{b} herein are each independently preferably a group comprising at least one acyl or sulfonyl-containing group and at least one nitrogen atom-containing group;
R₂ in both general formulas (II) and (IIa) is —B, i.e. is a group formed by means of dehydroxylation of a carboxy-containing carboxylic acid compound B₁ and is selected from R₄—Ar—R₃—CO—, wherein R₃ is selected from C₁-C₆ alkylene; —NH—, R₅NH—, or C₁-C₆ alkylene substituted with a C₁-C₆ alkoxyamide group; or a direct connection, i.e. the Ar group is directly linked to —CO—; R₃ is preferably methyl substituted or unsubstituted methylene, —C₂H₄—, or a direct connection; R₅ is C₁-C₆ alkylene; wherein the C₁-C₆ alkylene is substitutable by halogen (preferably halogen is one or two or more selected from a group consisting of fluorine, chlorine or bromine);
Ar is an aromatic ring group, preferably selected from a benzene ring; a naphthalene ring or an aryl heterocyclic ring; a benzene ring, a naphthalene ring, or an aryl heterocyclic ring or an aryl fused heterocyclic ring (here, the aryl heterocyclic ring being preferably a benzo nitrogen-containing or oxygen-containing benzo heterocyclic ring such as a benzopyrrole ring) substituted with one or more groups selected from halogen, a C₁-C₆ alkyl group, a C₁-C₆ alkoxy group, a C₁-C₆ acyl group, or a C₁-C₆ alkoxy group; Ar is more preferably an aryl heterocyclic ring containing a nitrogen atom; and
R₄ is halogen, C₁-C₆ alkyl, C₁-C₆ alkoxy, C₁-C₆ alkyl containing C₁-C₆ cycloalkanoyl, C₁-C₆ alkylamido or aryl fused heterocyclic amido, C₁-C₆ carbonyloxy, halogen substituted benzoyl, C₁-C₆ alkyl or halogen substituted or unsubstituted phenoxy, C₁-C₆ alkyl or halogen substituted or unsubstituted phenyl or aryl fused heterocyclic ring, C₁-C₆ alkyl or halogen substituted or unsubstituted phenylamino, or R₄ can also be absent;
wherein the C₁-C₆ alkoxy can also form a bridged ring with Ar.

In a specific embodiment, the anti-inflammatory compound, or the stereoisomer, tautomer, nitrogen oxide, metabolite, prodrug, pharmaceutically acceptable salt, or solvate thereof described above of the present invention, wherein Cy is substituted cyclohexyl or substituted piperidinyl; preferably, the substituted cyclohexyl group is a cyclohexyl group substituted with an amino group and a sulfonyl group, and the substituted piperidinyl group is a piperidinyl group substituted with an acyl group or a sulfonyl group and —CN.

Preferably, the compound, or a stereoisomer, tautomer, nitrogen oxide, metabolite, prodrug, pharmaceutically acceptable salt, or solvate thereof of the present invention is a coupling compound resulting from a condensation reaction of an amine compound A with a carboxylic acid compound B₁.

Preferably, the compound, or a stereoisomer, tautomer, nitrogen oxide, metabolite, prodrug, pharmaceutically acceptable salt, or solvate thereof described above of the present invention, wherein A- is a group after dehydrogenation of an amine compound selected from a group consisting of any one of the following groups: tofacitinib, baricitinib, oclacitinib, ruxolitinib, upadacitinib and delgocitinib:

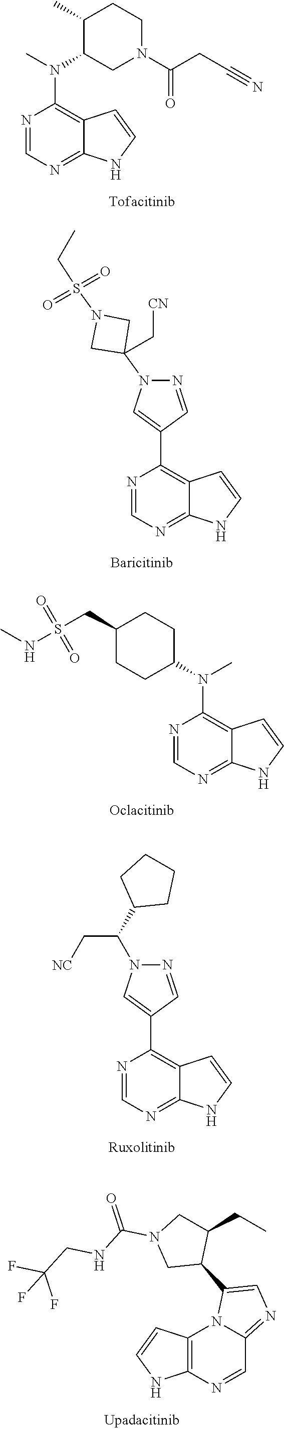

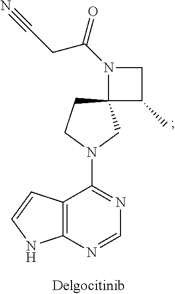 [SMILES: C[C@H]1CN(C(=O)CC#N)[C@]12CCN(c1ncnc3[nH]ccc13)C2]

preferably A is a group formed after dehydrogenation of tofacitinib, ruxolitinib, and baricitinib.

Still preferably, the anti-inflammatory compound or a stereoisomer, tautomer, nitrogen oxide, metabolite, prodrug, pharmaceutically acceptable salt, or solvate thereof of the present invention, wherein —B₁ is a group after dehydroxylation of a carboxylic acid moiety selected from a group consisting of ibuprofen, (S)-(+)-ibuprofen, naproxen, fenoprofen, flurbiprofen, loxoprofen, ketoprofen, diclofenac, etodolac, actarit, indomethacin, N-Boc-L-phenylglycine, aspirin, indobufen, mefenamic acid and tolfenamic acid:

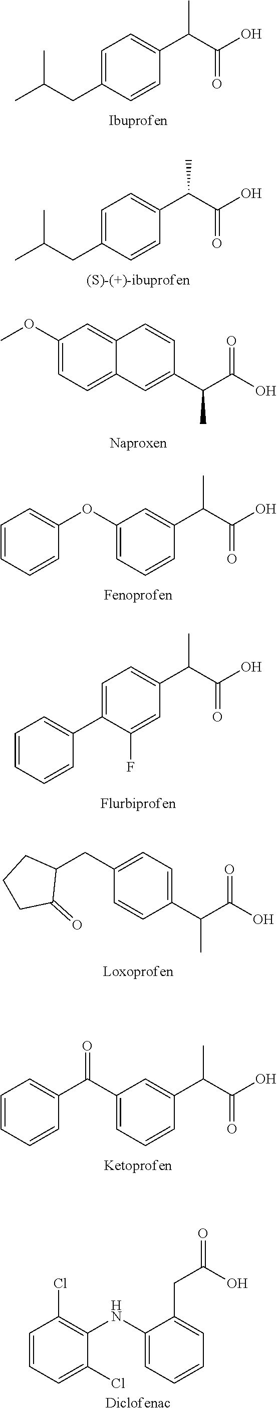

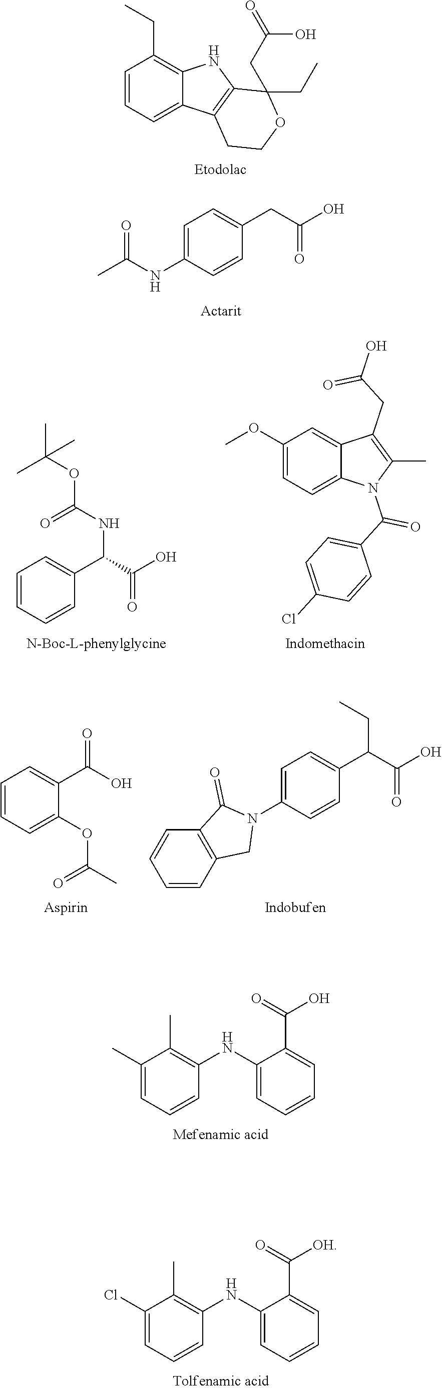

More preferably, the anti-inflammatory compound, or a stereoisomer, tautomer, N-oxide, metabolite, prodrug, pharmaceutically acceptable salt or solvate thereof of the present invention, wherein the compound is a coupled compound obtained by condensation reaction of an amine compound A selected from the group consisting of tofacitinib, baricitinib, upadacitinib, oclacitinib, and ruxolitinib with a carboxylic acid compound B₁ selected from the group consisting of ibuprofen, (S)-(+)-ibuprofen, naproxen, fenoprofen, flurbiprofen, loxoprofen, ketoprofen, etodolac, actarit and indomethacin; preferably, the amine compound is tofacitinib, ruxolitinib and baricitinib.

More preferably, the anti-inflammatory compound, or the stereoisomer, tautomer, nitrogen oxide, metabolite, prodrug, pharmaceutically acceptable salt, or solvate thereof of the present invention, wherein the compound is any one of the following specific compounds:

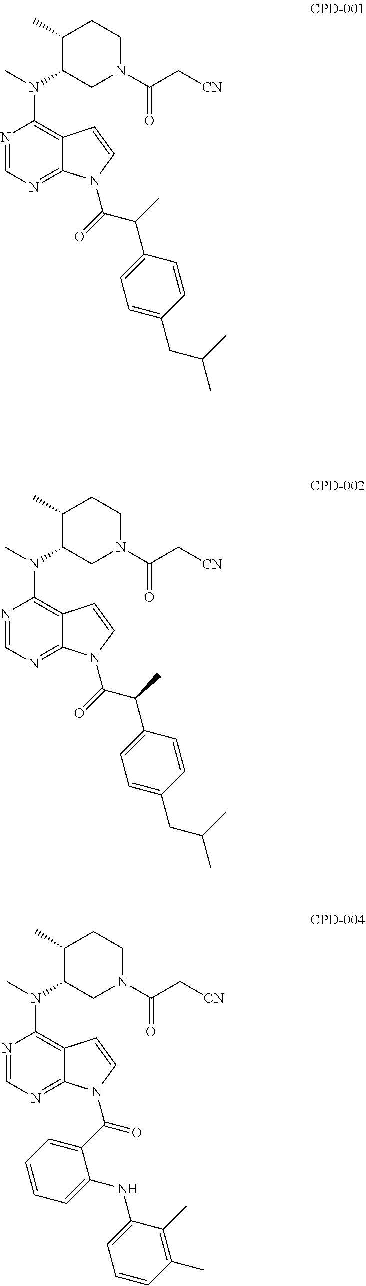 [SMILES: CC(C)Cc1ccc(C(C)C(=O)n2ccc3c(N(C)[C@H]4CN(C(=O)CC#N)CC[C@H]4C)ncnc32)cc1 ; CC(C)Cc1ccc([C@H](C)C(=O)n2ccc3c(N(C)[C@H]4CN(C(=O)CC#N)CC[C@H]4C)ncnc32)cc1 ; Cc1cccc(Nc2ccccc2C(=O)n2ccc3c(N(C)[C@H]4CN(C(=O)CC#N)CC[C@H]4C)ncnc32)c1C]

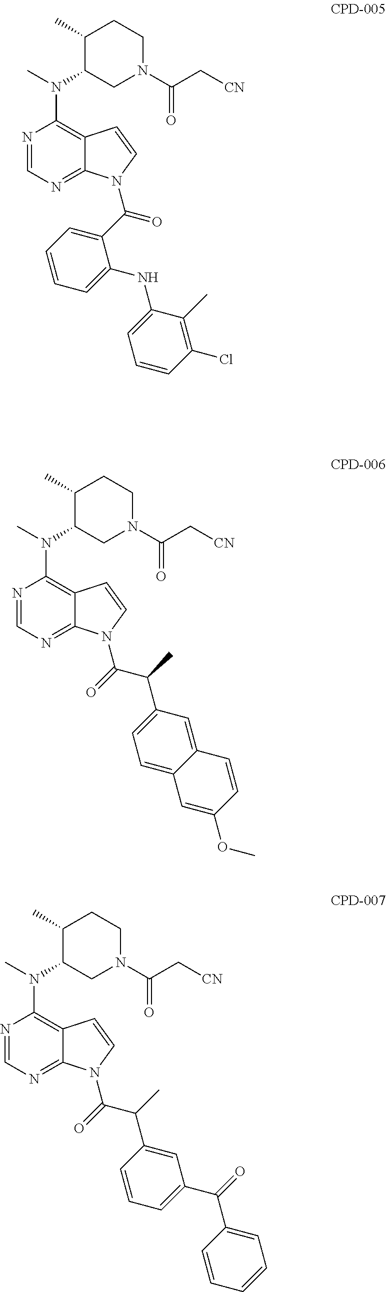 [SMILES: Cc1c(Cl)cccc1Nc1ccccc1C(=O)n1ccc2c(N(C)[C@H]3CN(C(=O)CC#N)CC[C@H]3C)ncnc21 ; COc1ccc2cc([C@H](C)C(=O)n3ccc4c(N(C)[C@H]5CN(C(=O)CC#N)CC[C@H]5C)ncnc43)ccc2c1 ; CC(C(=O)n1ccc2c(N(C)[C@H]3CN(C(=O)CC#N)CC[C@H]3C)ncnc21)c1cccc(C(=O)c2ccccc2)c1]

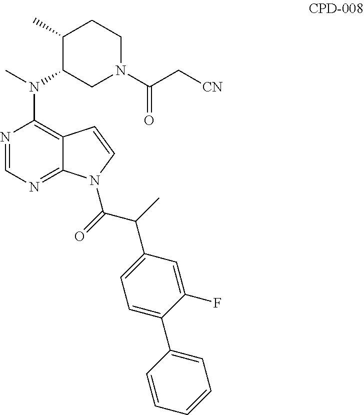 [SMILES: CC(C(=O)n1ccc2c(N(C)[C@H]3CN(C(=O)CC#N)CC[C@H]3C)ncnc21)c1ccc(-c2ccccc2)c(F)c1]

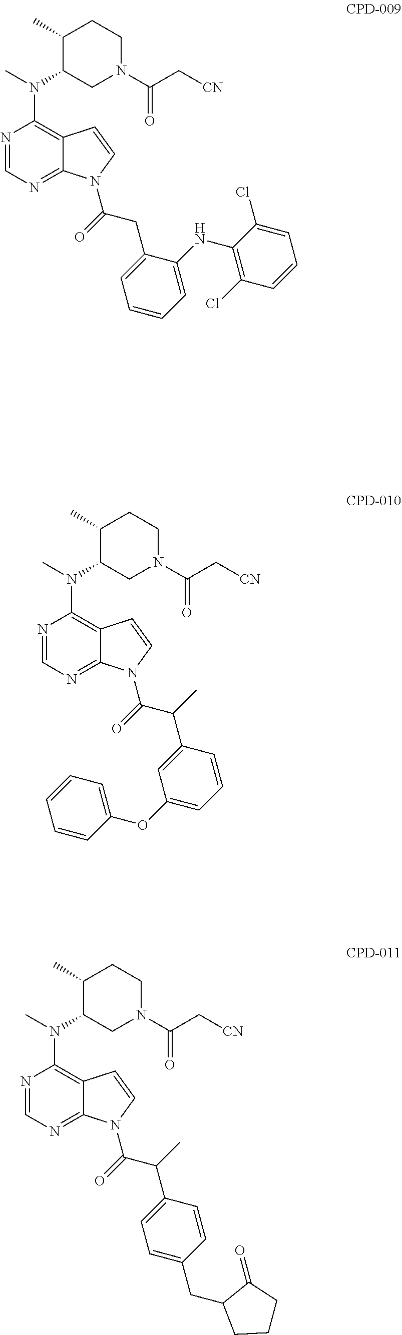 [SMILES: C[C@@H]1CCN(C(=O)CC#N)C[C@@H]1N(C)c1ncnc2c1ccn2C(=O)Cc1ccccc1Nc1c(Cl)cccc1Cl ; CC(C(=O)n1ccc2c(N(C)[C@H]3CN(C(=O)CC#N)CC[C@H]3C)ncnc21)c1cccc(Oc2ccccc2)c1 ; CC(C(=O)n1ccc2c(N(C)[C@H]3CN(C(=O)CC#N)CC[C@H]3C)ncnc21)c1ccc(CC2CCCC2=O)cc1]

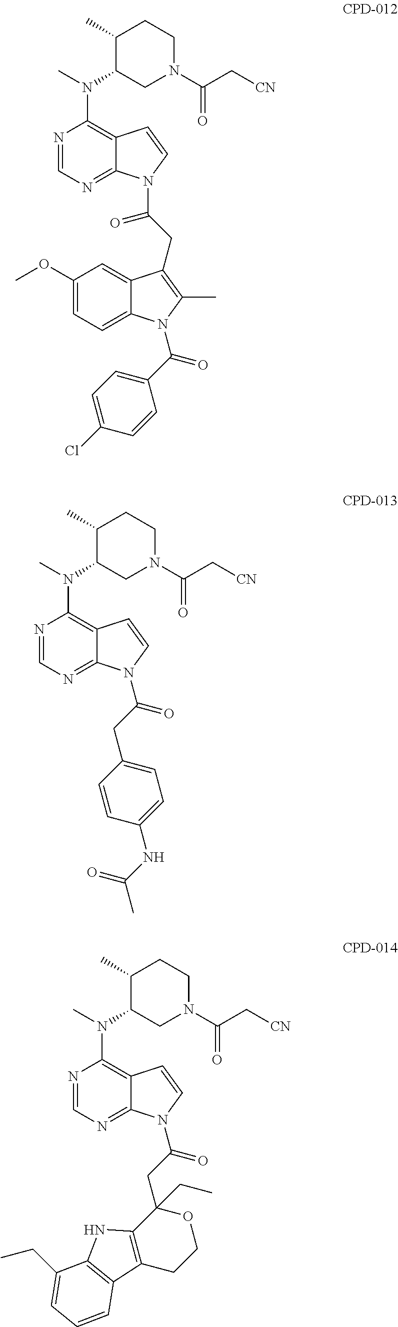 [SMILES: COc1ccc2c(c1)c(CC(=O)n1ccc3c(N(C)[C@H]4CN(C(=O)CC#N)CC[C@H]4C)ncnc31)c(C)n2C(=O)c1ccc(Cl)cc1 ; CC(=O)Nc1ccc(CC(=O)n2ccc3c(N(C)[C@H]4CN(C(=O)CC#N)CC[C@H]4C)ncnc32)cc1 ; CCc1cccc2c3c([nH]c12)C(CC)(CC(=O)n1ccc2c(N(C)[C@H]4CN(C(=O)CC#N)CC[C@H]4C)ncnc21)OCC3]

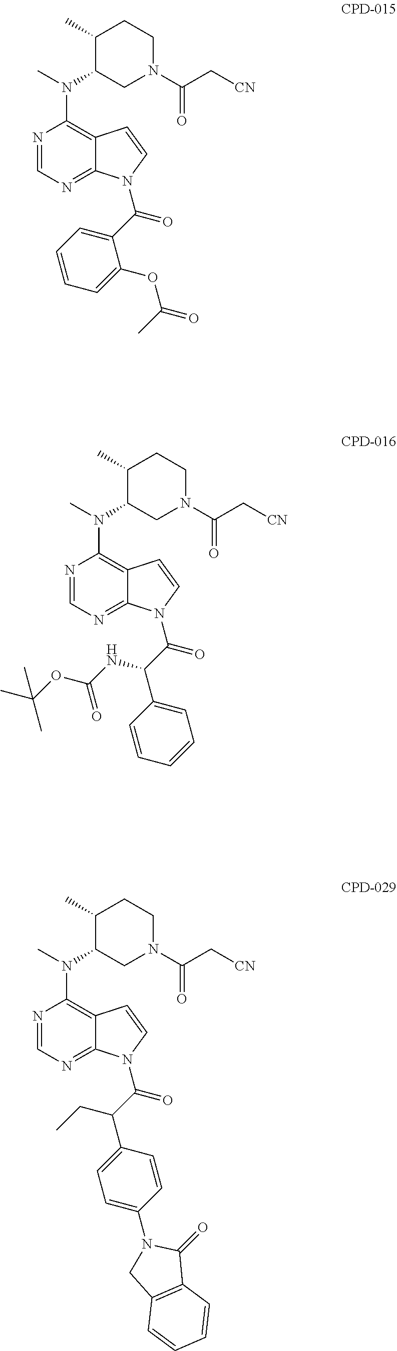 [SMILES: CC(=O)Oc1ccccc1C(=O)n1ccc2c(N(C)[C@H]3CN(C(=O)CC#N)CC[C@H]3C)ncnc21 ; C[C@@H]1CCN(C(=O)CC#N)C[C@@H]1N(C)c1ncnc2c1ccn2C(=O)[C@@H](NC(=O)OC(C)(C)C)c1ccccc1 ; CCC(C(=O)n1ccc2c(N(C)[C@H]3CN(C(=O)CC#N)CC[C@H]3C)ncnc21)c1ccc(N2Cc3ccccc3C2=O)cc1]

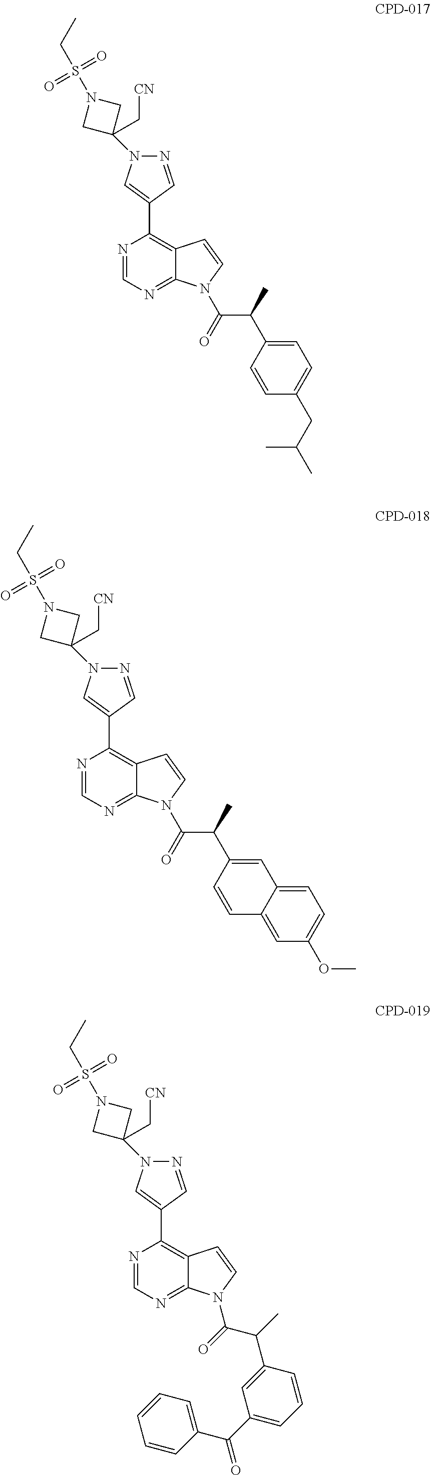 [SMILES: CCS(=O)(=O)N1CC(CC#N)(n2cc(-c3ncnc4c3ccn4C(=O)[C@@H](C)c3ccc(CC(C)C)cc3)cn2)C1 ; CCS(=O)(=O)N1CC(CC#N)(n2cc(-c3ncnc4c3ccn4C(=O)[C@@H](C)c3ccc4cc(OC)ccc4c3)cn2)C1 ; CCS(=O)(=O)N1CC(CC#N)(n2cc(-c3ncnc4c3ccn4C(=O)C(C)c3cccc(C(=O)c4ccccc4)c3)cn2)C1]

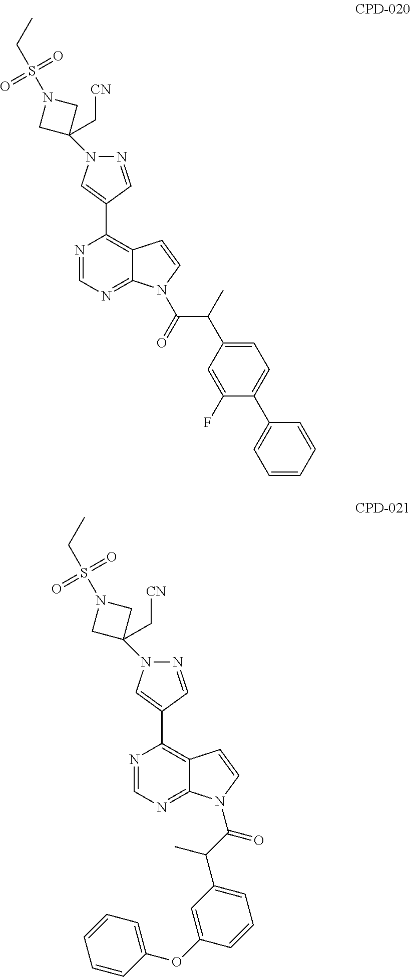 [SMILES: CCS(=O)(=O)N1CC(CC#N)(n2cc(-c3ncnc4c3ccn4C(=O)C(C)c3ccc(-c4ccccc4)c(F)c3)cn2)C1 ; CCS(=O)(=O)N1CC(CC#N)(n2cc(-c3ncnc4c3ccn4C(=O)C(C)c3cccc(Oc4ccccc4)c3)cn2)C1]

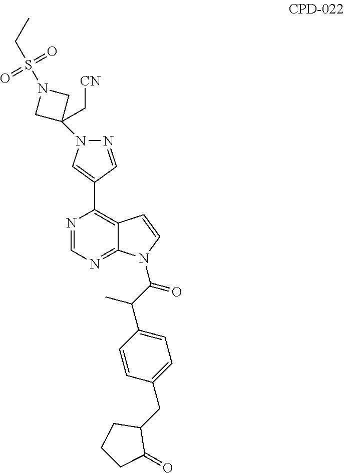 [SMILES: CCS(=O)(=O)N1CC(CC#N)(n2cc(-c3ncnc4c3ccn4C(=O)C(C)c3ccc(CC4CCCC4=O)cc3)cn2)C1]

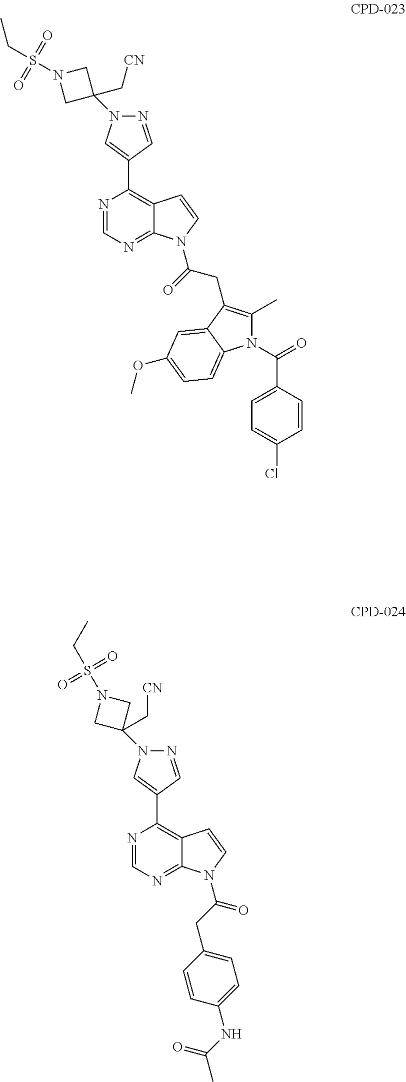 [SMILES: CCS(=O)(=O)N1CC(CC#N)(n2cc(-c3ncnc4c3ccn4C(=O)Cc3c(C)n(C(=O)c4ccc(Cl)cc4)c4ccc(OC)cc34)cn2)C1 ; CCS(=O)(=O)N1CC(CC#N)(n2cc(-c3ncnc4c3ccn4C(=O)Cc3ccc(NC(C)=O)cc3)cn2)C1]

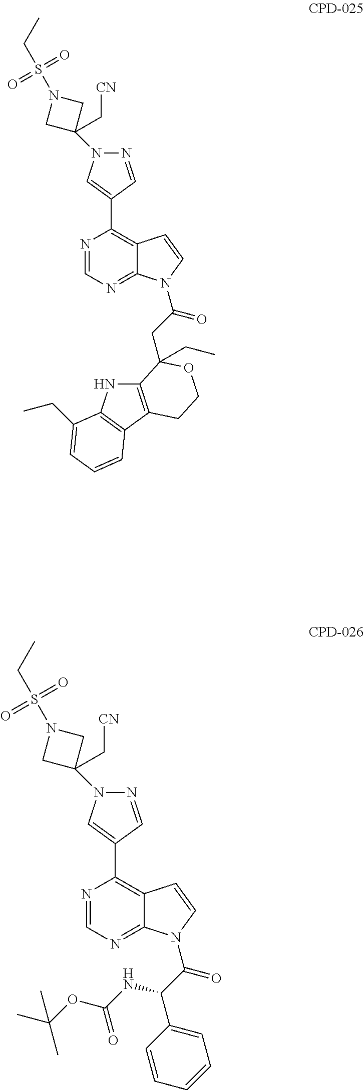 [SMILES: CCc1cccc2c3c([nH]c12)C(CC)(CC(=O)n1ccc2c(-c4cnn(C5(CC#N)CN(S(=O)(=O)CC)C5)c4)ncnc21)OCC3 ; CCS(=O)(=O)N1CC(CC#N)(n2cc(-c3ncnc4c3ccn4C(=O)[C@@H](NC(=O)OC(C)(C)C)c3ccccc3)cn2)C1]

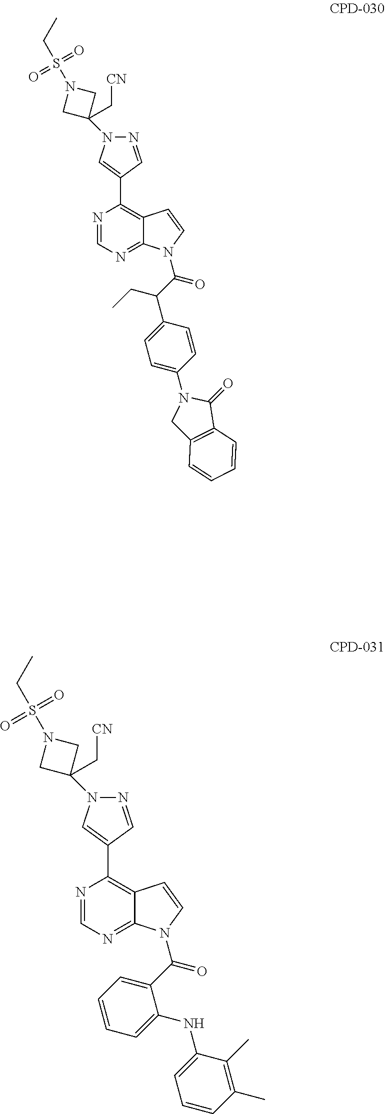 [SMILES: CCC(C(=O)n1ccc2c(-c3cnn(C4(CC#N)CN(S(=O)(=O)CC)C4)c3)ncnc21)c1ccc(N2Cc3ccccc3C2=O)cc1 ; CCS(=O)(=O)N1CC(CC#N)(n2cc(-c3ncnc4c3ccn4C(=O)c3ccccc3Nc3cccc(C)c3C)cn2)C1]

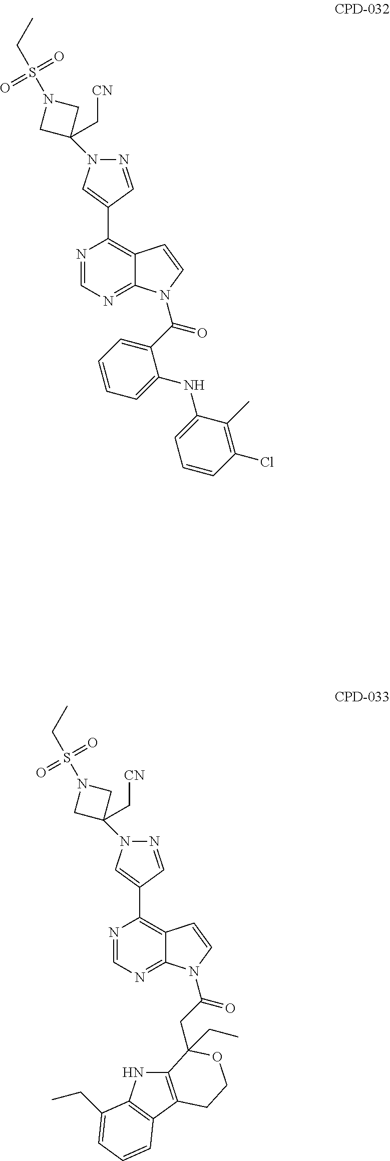 [SMILES: CCS(=O)(=O)N1CC(CC#N)(n2cc(-c3ncnc4c3ccn4C(=O)c3ccccc3Nc3cccc(Cl)c3C)cn2)C1 ; CCc1cccc2c3c([nH]c12)C(CC)(CC(=O)n1ccc2c(-c4cnn(C5(CC#N)CN(S(=O)(=O)CC)C5)c4)ncnc21)OCC3]

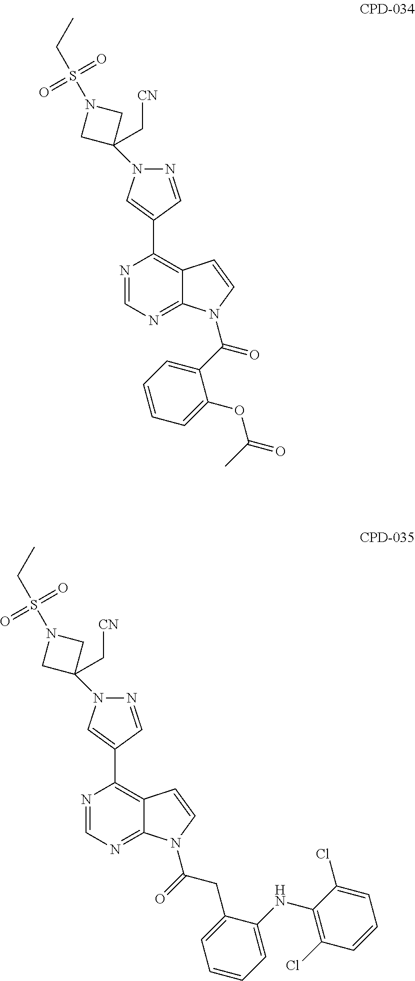 [SMILES: CCS(=O)(=O)N1CC(CC#N)(n2cc(-c3ncnc4c3ccn4C(=O)c3ccccc3OC(C)=O)cn2)C1 ; CCS(=O)(=O)N1CC(CC#N)(n2cc(-c3ncnc4c3ccn4C(=O)Cc3ccccc3Nc3c(Cl)cccc3Cl)cn2)C1]

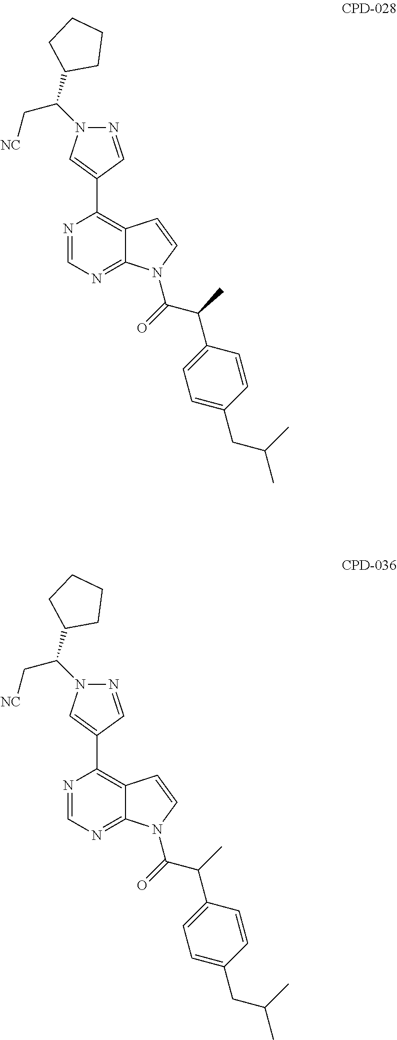 [SMILES: CC(C)Cc1ccc([C@H](C)C(=O)n2ccc3c(-c4cnn([C@H](CC#N)C5CCCC5)c4)ncnc32)cc1 ; CC(C)Cc1ccc(C(C)C(=O)n2ccc3c(-c4cnn([C@H](CC#N)C5CCCC5)c4)ncnc32)cc1]

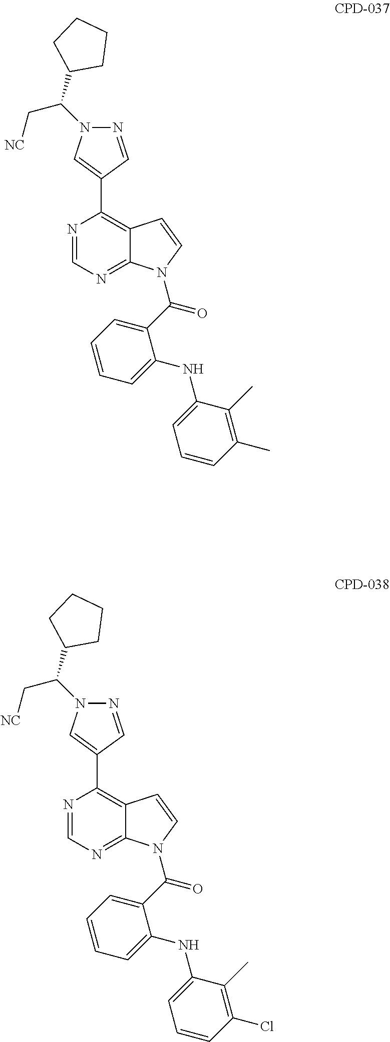 [SMILES: Cc1cccc(Nc2ccccc2C(=O)n2ccc3c(-c4cnn([C@H](CC#N)C5CCCC5)c4)ncnc32)c1C ; Cc1c(Cl)cccc1Nc1ccccc1C(=O)n1ccc2c(-c3cnn([C@H](CC#N)C4CCCC4)c3)ncnc21]

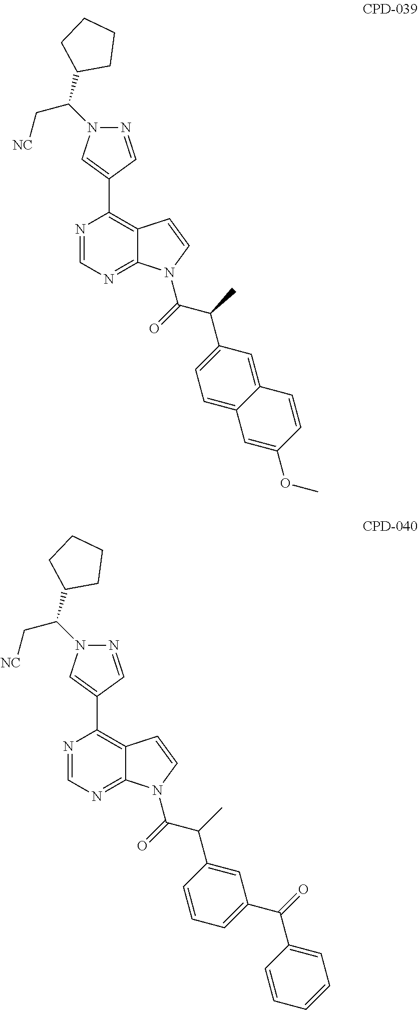 [SMILES: COc1ccc2cc([C@H](C)C(=O)n3ccc4c(-c5cnn([C@H](CC#N)C6CCCC6)c5)ncnc43)ccc2c1 ; CC(C(=O)n1ccc2c(-c3cnn([C@H](CC#N)C4CCCC4)c3)ncnc21)c1cccc(C(=O)c2ccccc2)c1]

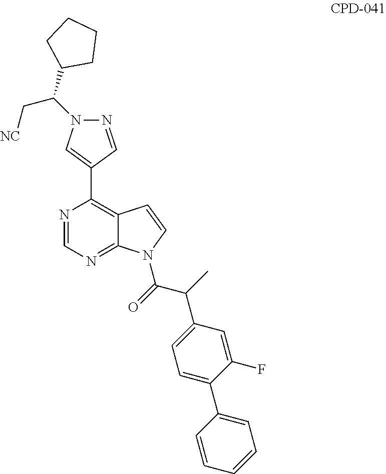 [SMILES: CC(C(=O)n1ccc2c(-c3cnn([C@H](CC#N)C4CCCC4)c3)ncnc21)c1ccc(-c2ccccc2)c(F)c1]

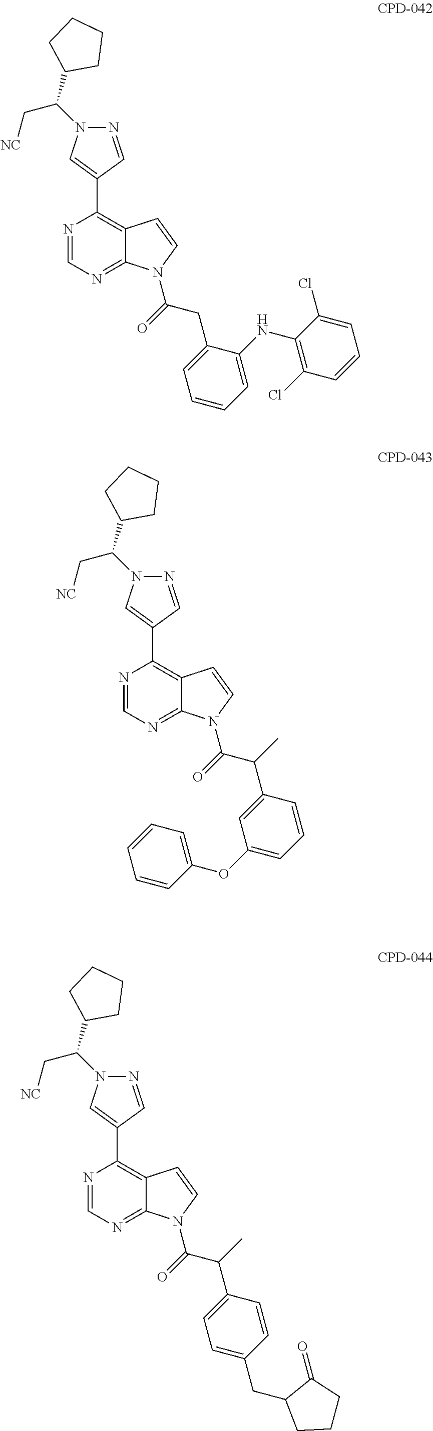 [SMILES: N#CC[C@H](C1CCCC1)n1cc(-c2ncnc3c2ccn3C(=O)Cc2ccccc2Nc2c(Cl)cccc2Cl)cn1 ; CC(C(=O)n1ccc2c(-c3cnn([C@H](CC#N)C4CCCC4)c3)ncnc21)c1cccc(Oc2ccccc2)c1 ; CC(C(=O)n1ccc2c(-c3cnn([C@H](CC#N)C4CCCC4)c3)ncnc21)c1ccc(CC2CCCC2=O)cc1]

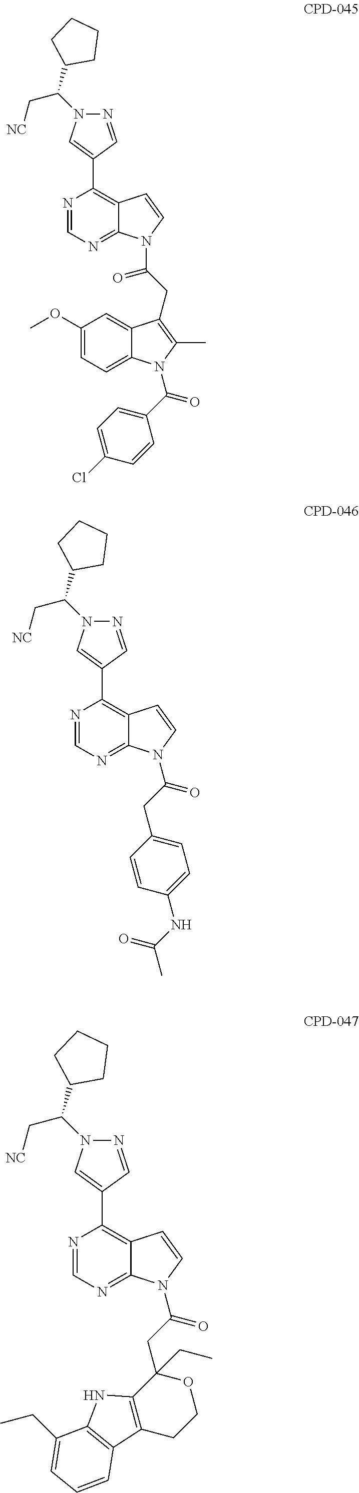

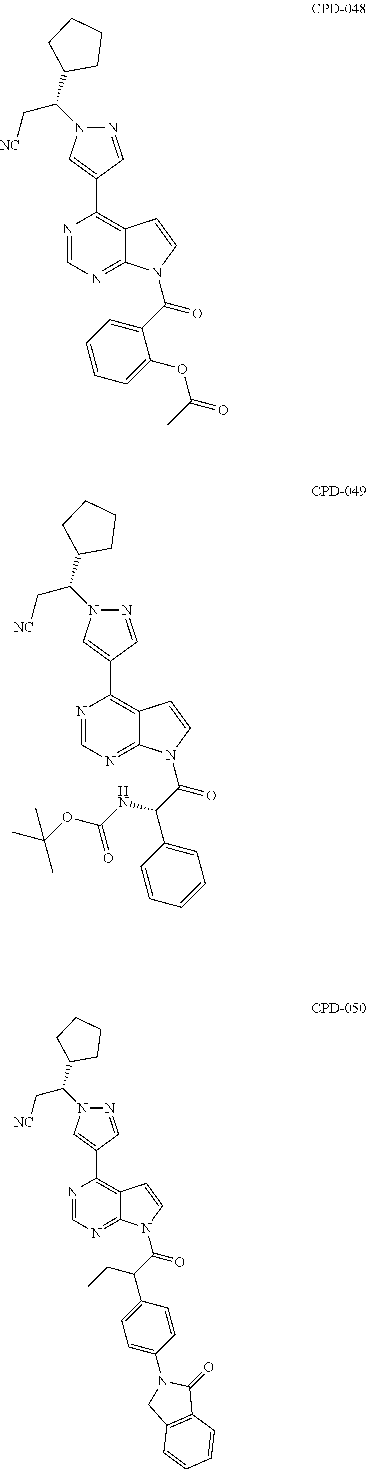 [SMILES: CC(=O)Oc1ccccc1C(=O)n1ccc2c(-c3cnn([C@H](CC#N)C4CCCC4)c3)ncnc21 ; CC(C)(C)OC(=O)N[C@H](C(=O)n1ccc2c(-c3cnn([C@H](CC#N)C4CCCC4)c3)ncnc21)c1ccccc1 ; CCC(C(=O)n1ccc2c(-c3cnn([C@H](CC#N)C4CCCC4)c3)ncnc21)c1ccc(N2Cc3ccccc3C2=O)cc1]

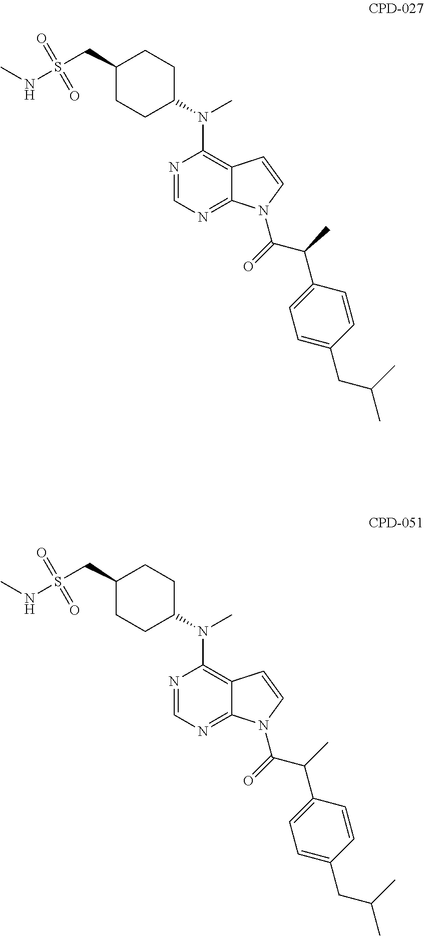

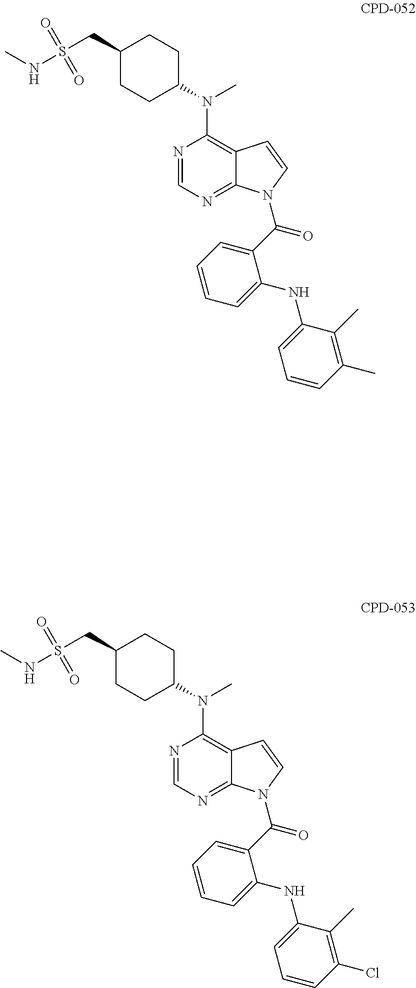

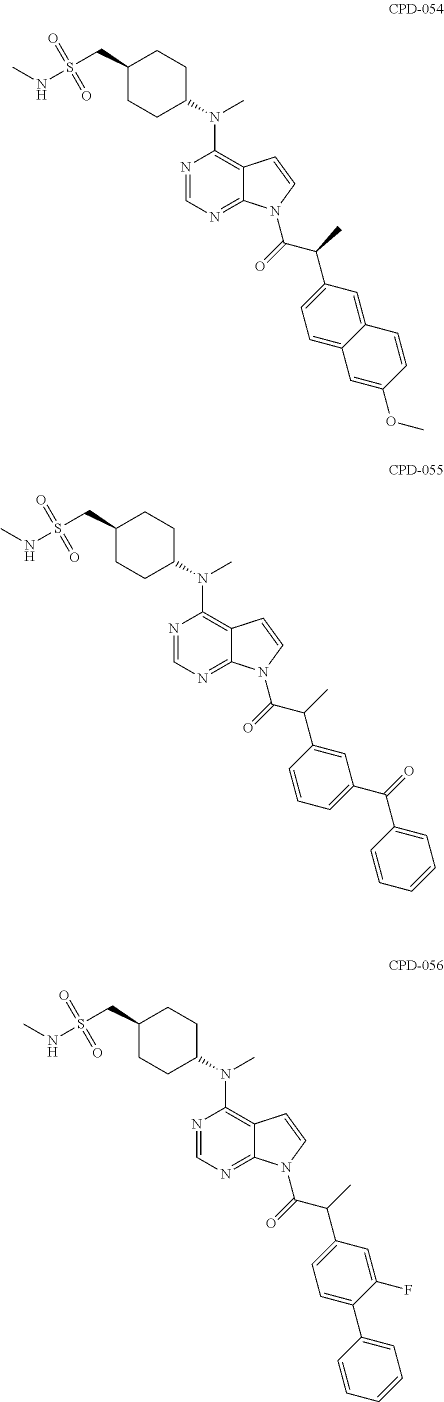

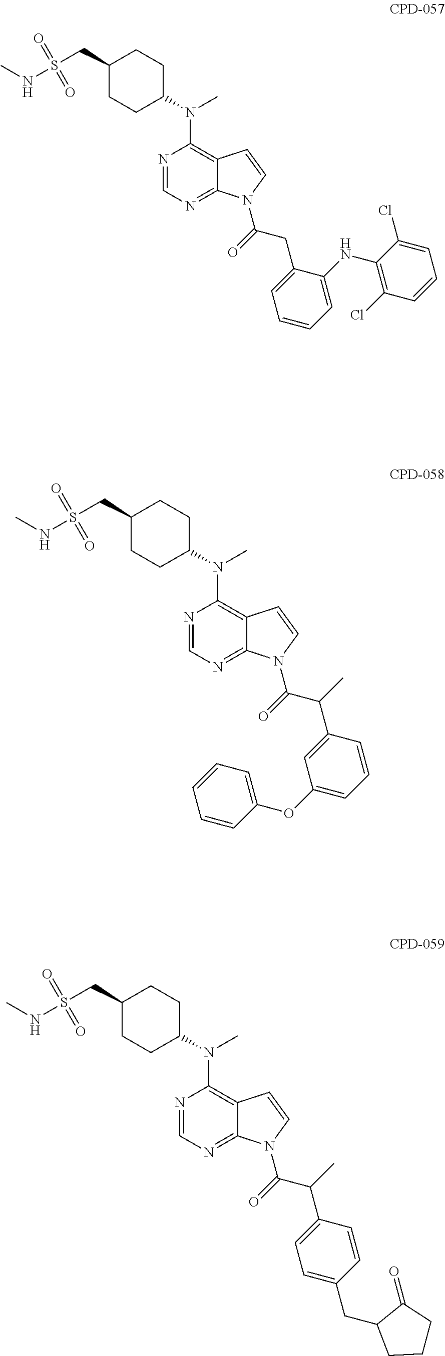

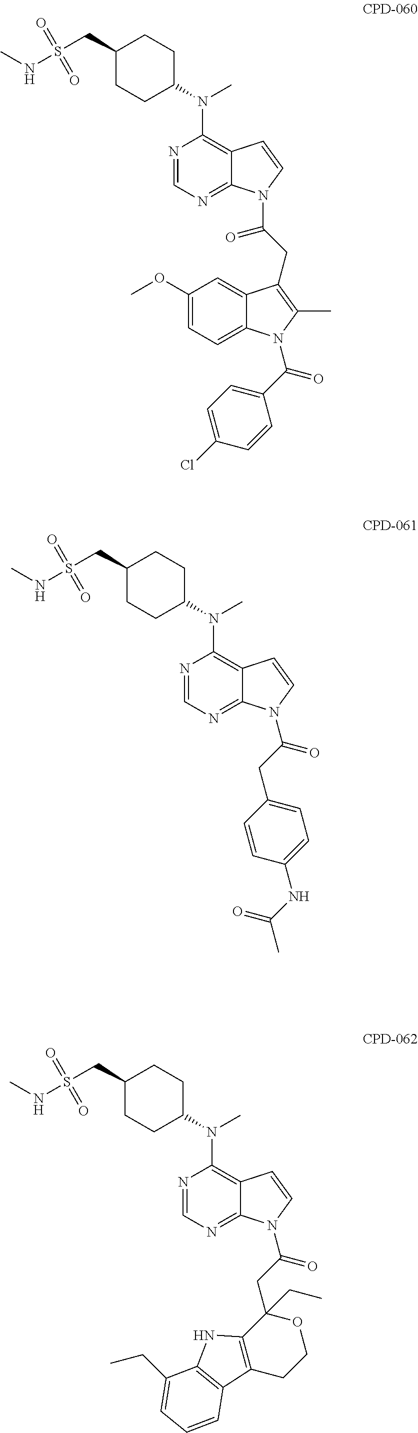

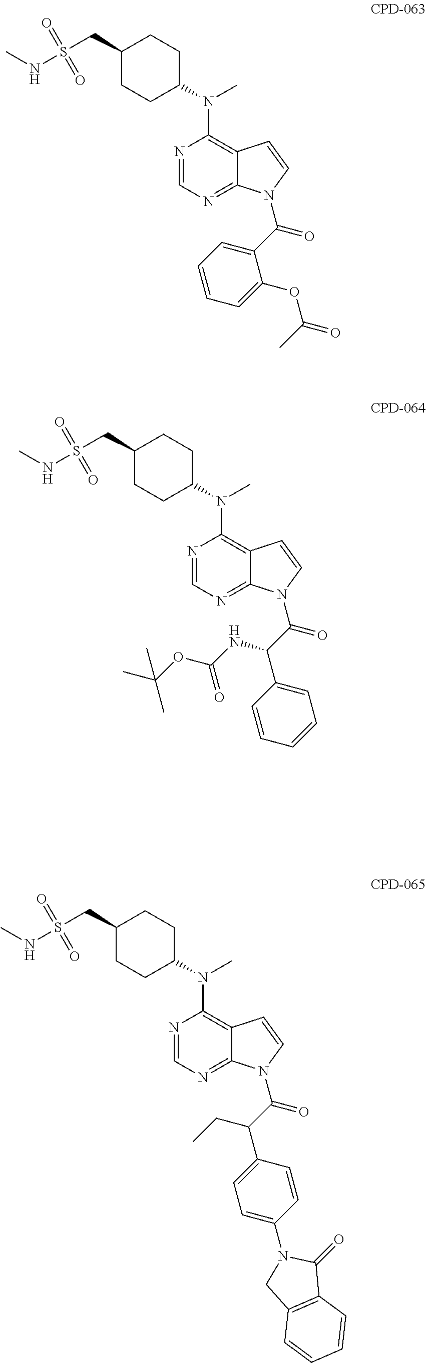

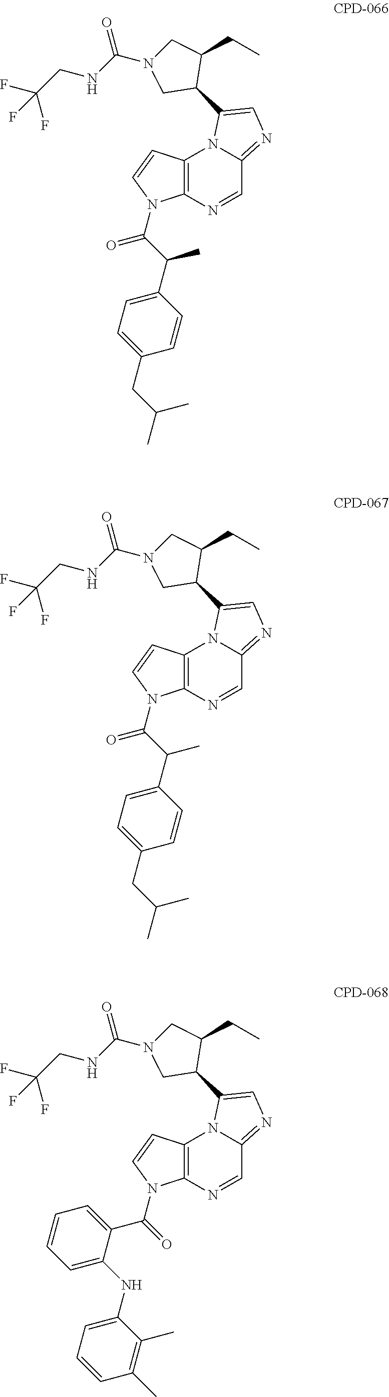 [SMILES: CC[C@@H]1CN(C(=O)NCC(F)(F)F)C[C@@H]1c1cnc2cnc3c(ccn3C(=O)[C@@H](C)c3ccc(CC(C)C)cc3)n12 ; CC[C@@H]1CN(C(=O)NCC(F)(F)F)C[C@@H]1c1cnc2cnc3c(ccn3C(=O)C(C)c3ccc(CC(C)C)cc3)n12 ; CC[C@@H]1CN(C(=O)NCC(F)(F)F)C[C@@H]1c1cnc2cnc3c(ccn3C(=O)c3ccccc3Nc3cccc(C)c3C)n12]

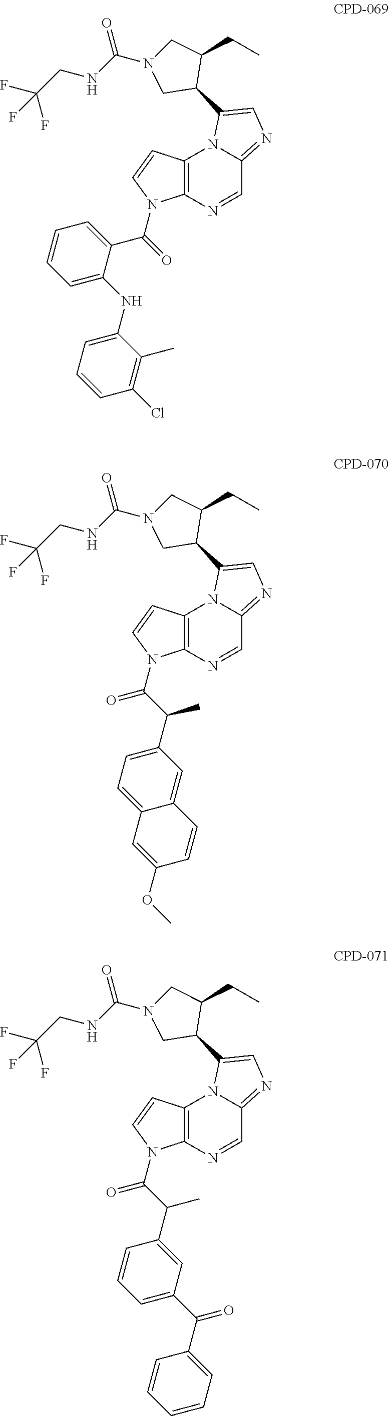 [SMILES: CC[C@@H]1CN(C(=O)NCC(F)(F)F)C[C@@H]1c1cnc2cnc3c(ccn3C(=O)c3ccccc3Nc3cccc(Cl)c3C)n12 ; CC[C@@H]1CN(C(=O)NCC(F)(F)F)C[C@@H]1c1cnc2cnc3c(ccn3C(=O)[C@@H](C)c3ccc4cc(OC)ccc4c3)n12 ; CC[C@@H]1CN(C(=O)NCC(F)(F)F)C[C@@H]1c1cnc2cnc3c(ccn3C(=O)C(C)c3cccc(C(=O)c4ccccc4)c3)n12]

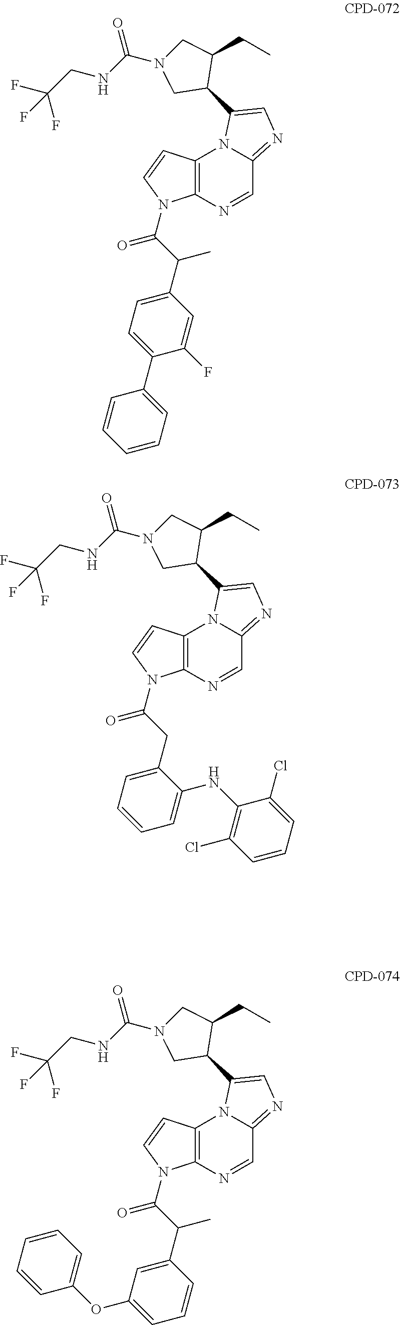 [SMILES: CC[C@@H]1CN(C(=O)NCC(F)(F)F)C[C@@H]1c1cnc2cnc3c(ccn3C(=O)C(C)c3ccc(-c4ccccc4)c(F)c3)n12 ; CC[C@@H]1CN(C(=O)NCC(F)(F)F)C[C@@H]1c1cnc2cnc3c(ccn3C(=O)Cc3ccccc3Nc3c(Cl)cccc3Cl)n12 ; CC[C@@H]1CN(C(=O)NCC(F)(F)F)C[C@@H]1c1cnc2cnc3c(ccn3C(=O)C(C)c3cccc(Oc4ccccc4)c3)n12]

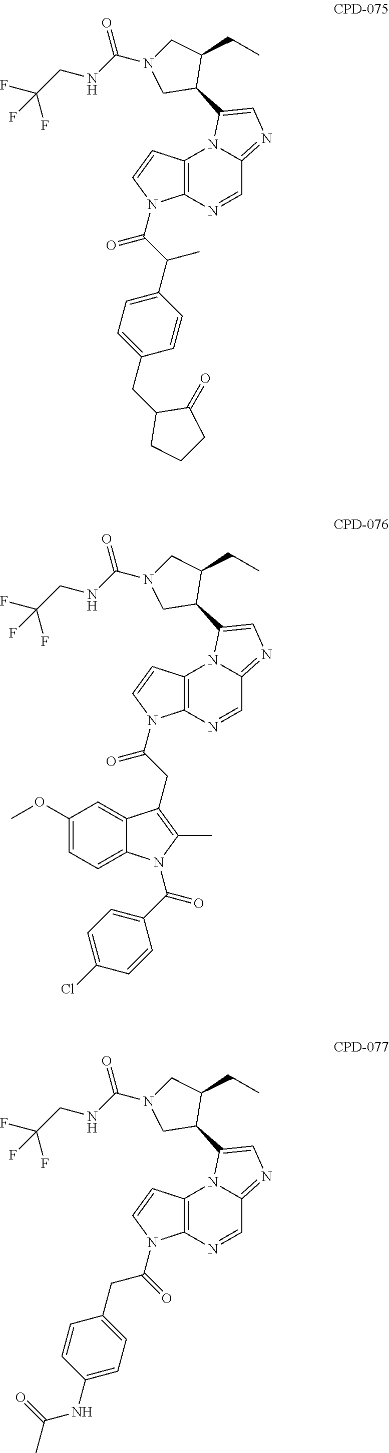 [SMILES: CC[C@@H]1CN(C(=O)NCC(F)(F)F)C[C@@H]1c1cnc2cnc3c(ccn3C(=O)C(C)c3ccc(CC4CCCC4=O)cc3)n12 ; CC[C@@H]1CN(C(=O)NCC(F)(F)F)C[C@@H]1c1cnc2cnc3c(ccn3C(=O)Cc3c(C)n(C(=O)c4ccc(Cl)cc4)c4ccc(OC)cc34)n12 ; CC[C@@H]1CN(C(=O)NCC(F)(F)F)C[C@@H]1c1cnc2cnc3c(ccn3C(=O)Cc3ccc(NC(C)=O)cc3)n12]

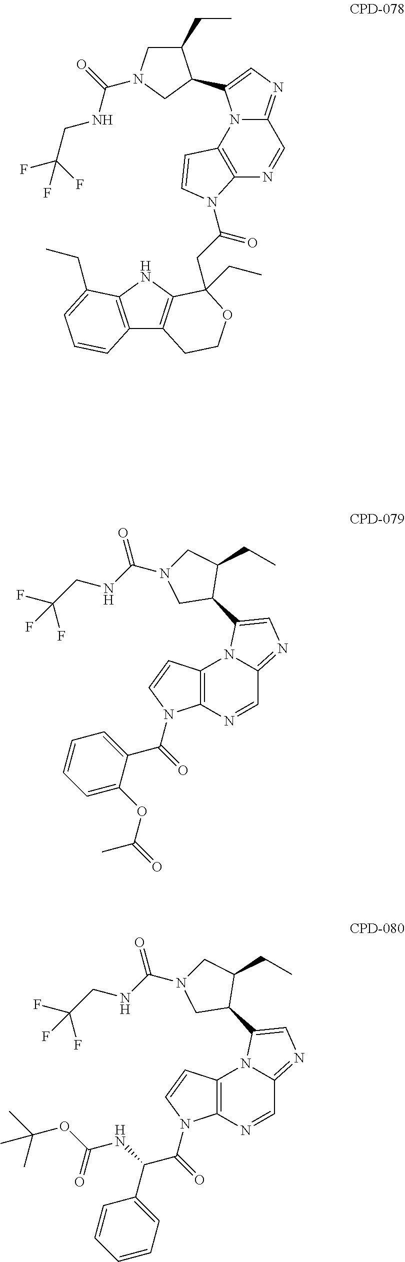 [SMILES: CCc1cccc2c3c([nH]c12)C(CC)(CC(=O)n1ccc2c1ncc1ncc([C@H]4CN(C(=O)NCC(F)(F)F)C[C@H]4CC)n12)OCC3 ; CC[C@@H]1CN(C(=O)NCC(F)(F)F)C[C@@H]1c1cnc2cnc3c(ccn3C(=O)c3ccccc3OC(C)=O)n12 ; CC[C@@H]1CN(C(=O)NCC(F)(F)F)C[C@@H]1c1cnc2cnc3c(ccn3C(=O)[C@@H](NC(=O)OC(C)(C)C)c3ccccc3)n12]

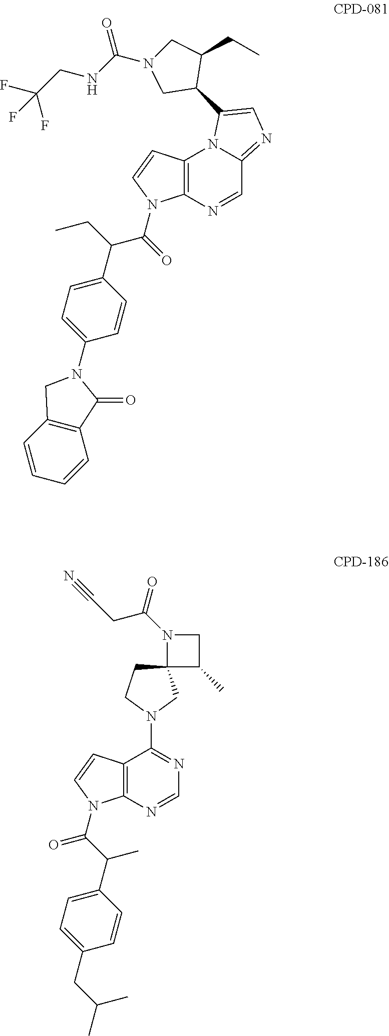 [SMILES: CCC(C(=O)n1ccc2c1ncc1ncc([C@H]3CN(C(=O)NCC(F)(F)F)C[C@H]3CC)n12)c1ccc(N2Cc3ccccc3C2=O)cc1 ; CC(C)Cc1ccc(C(C)C(=O)n2ccc3c(N4CC[C@]5(C4)[C@@H](C)CN5C(=O)CC#N)ncnc32)cc1]

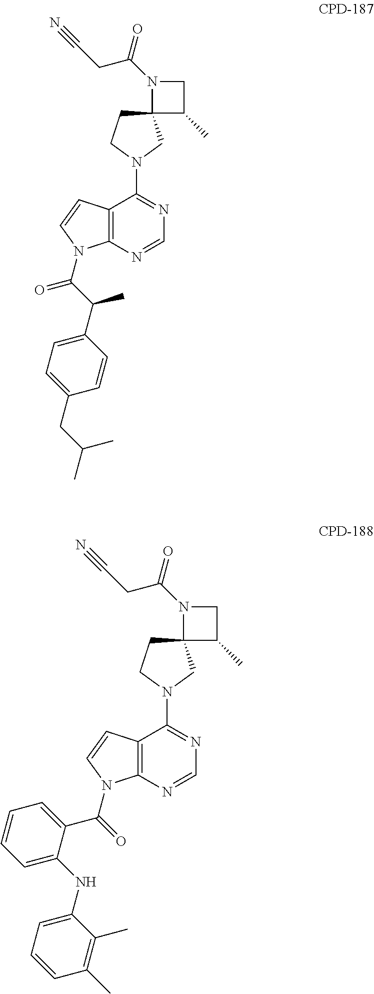 [SMILES: CC(C)Cc1ccc([C@H](C)C(=O)n2ccc3c(N4CC[C@]5(C4)[C@@H](C)CN5C(=O)CC#N)ncnc32)cc1 ; Cc1cccc(Nc2ccccc2C(=O)n2ccc3c(N4CC[C@]5(C4)[C@@H](C)CN5C(=O)CC#N)ncnc32)c1C]

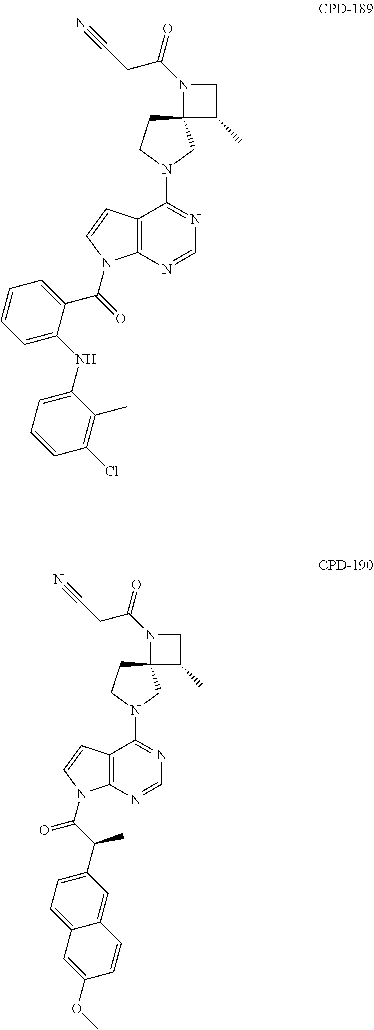 [SMILES: Cc1c(Cl)cccc1Nc1ccccc1C(=O)n1ccc2c(N3CC[C@]4(C3)[C@@H](C)CN4C(=O)CC#N)ncnc21 ; COc1ccc2cc([C@H](C)C(=O)n3ccc4c(N5CC[C@]6(C5)[C@@H](C)CN6C(=O)CC#N)ncnc43)ccc2c1]

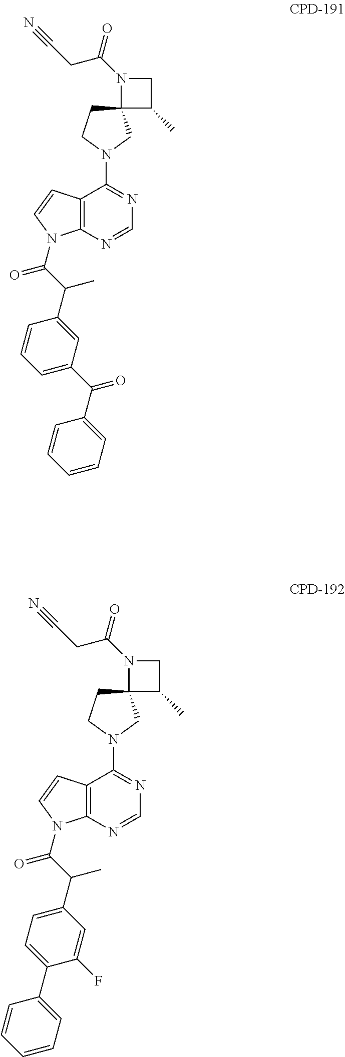 [SMILES: CC(C(=O)n1ccc2c(N3CC[C@]4(C3)[C@@H](C)CN4C(=O)CC#N)ncnc21)c1cccc(C(=O)c2ccccc2)c1 ; CC(C(=O)n1ccc2c(N3CC[C@]4(C3)[C@@H](C)CN4C(=O)CC#N)ncnc21)c1ccc(-c2ccccc2)c(F)c1]

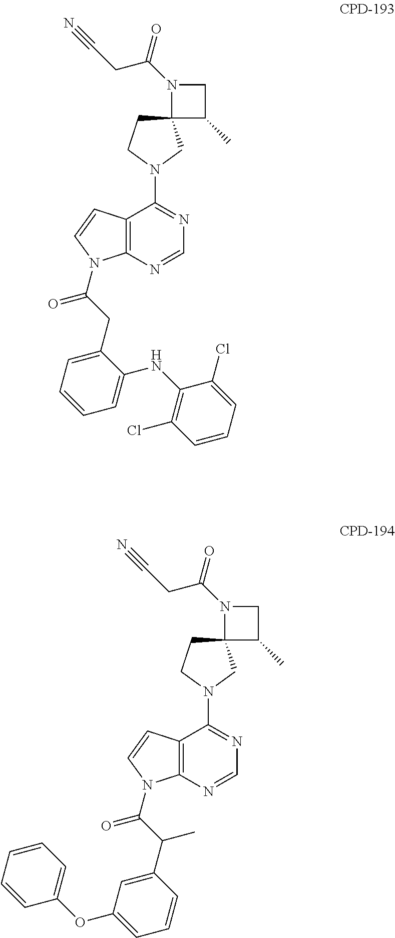 [SMILES: C[C@H]1CN(C(=O)CC#N)[C@]12CCN(c1ncnc3c1ccn3C(=O)Cc1ccccc1Nc1c(Cl)cccc1Cl)C2 ; CC(C(=O)n1ccc2c(N3CC[C@]4(C3)[C@@H](C)CN4C(=O)CC#N)ncnc21)c1cccc(Oc2ccccc2)c1]

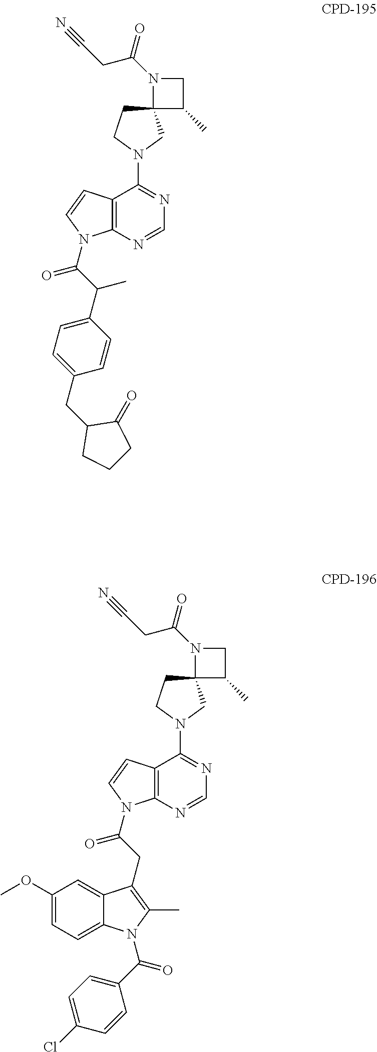

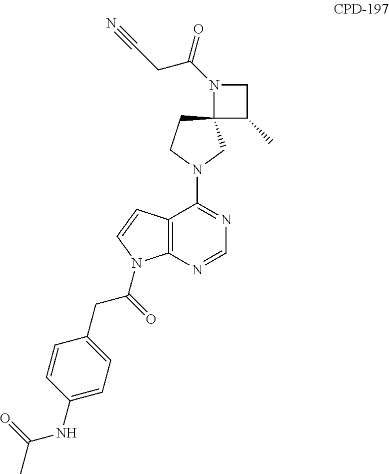 [SMILES: CC(=O)Nc1ccc(CC(=O)n2ccc3c(N4CC[C@]5(C4)[C@@H](C)CN5C(=O)CC#N)ncnc32)cc1]

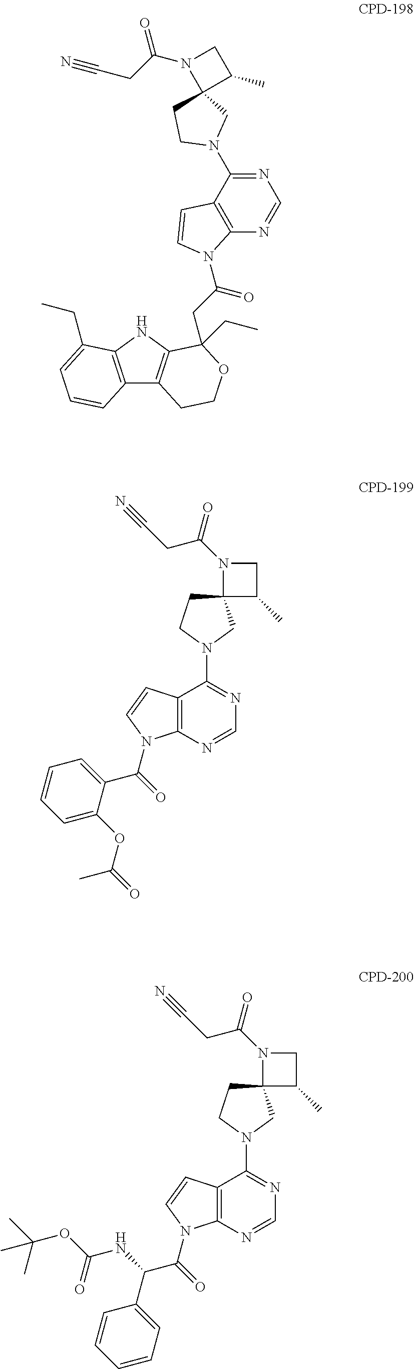

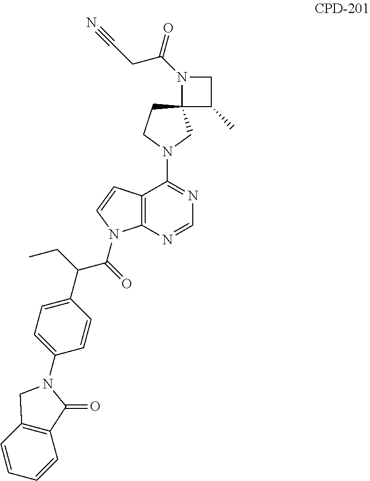 [SMILES: CCC(C(=O)n1ccc2c(N3CC[C@]4(C3)[C@@H](C)CN4C(=O)CC#N)ncnc21)c1ccc(N2Cc3ccccc3C2=O)cc1]

More preferably, the anti-inflammatory compound, or the stereoisomer, tautomer, nitrogen oxide, metabolite, prodrug, pharmaceutically acceptable salt, or solvate thereof of the present invention, wherein the compound is any one of the following specific compounds:

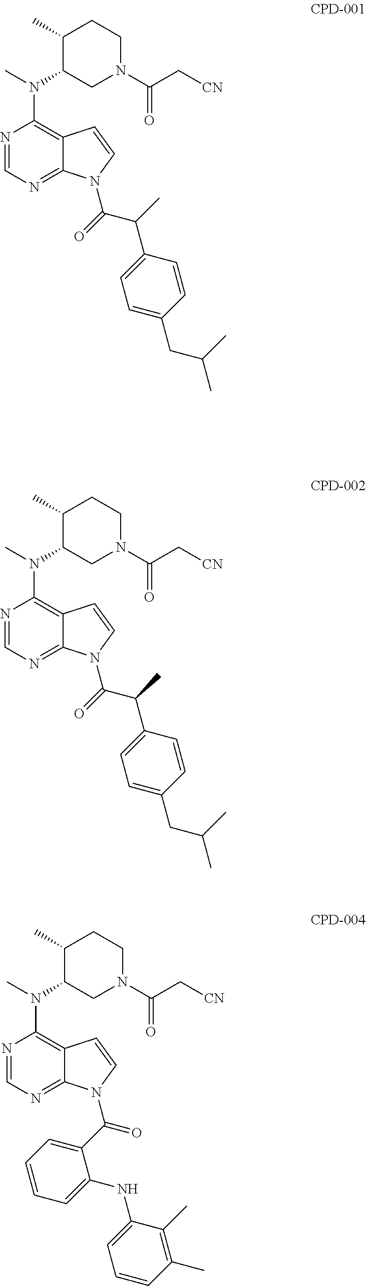 [SMILES: CC(C)Cc1ccc(C(C)C(=O)n2ccc3c(N(C)[C@H]4CN(C(=O)CC#N)CC[C@H]4C)ncnc32)cc1 ; CC(C)Cc1ccc([C@H](C)C(=O)n2ccc3c(N(C)[C@H]4CN(C(=O)CC#N)CC[C@H]4C)ncnc32)cc1 ; Cc1cccc(Nc2ccccc2C(=O)n2ccc3c(N(C)[C@H]4CN(C(=O)CC#N)CC[C@H]4C)ncnc32)c1C]

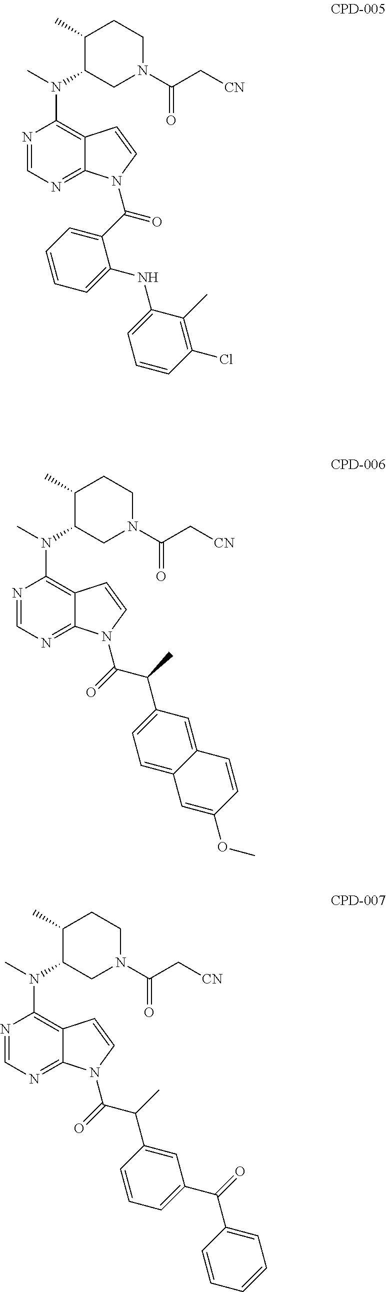 [SMILES: Cc1c(Cl)cccc1Nc1ccccc1C(=O)n1ccc2c(N(C)[C@H]3CN(C(=O)CC#N)CC[C@H]3C)ncnc21 ; COc1ccc2cc([C@H](C)C(=O)n3ccc4c(N(C)[C@H]5CN(C(=O)CC#N)CC[C@H]5C)ncnc43)ccc2c1 ; CC(C(=O)n1ccc2c(N(C)[C@H]3CN(C(=O)CC#N)CC[C@H]3C)ncnc21)c1cccc(C(=O)c2ccccc2)c1]

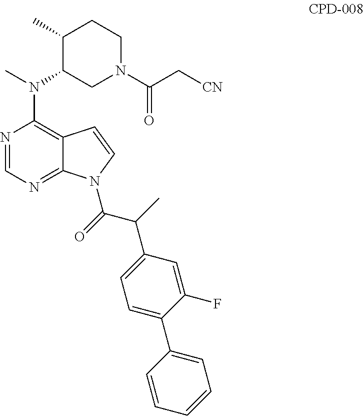 [SMILES: CC(C(=O)n1ccc2c(N(C)[C@H]3CN(C(=O)CC#N)CC[C@H]3C)ncnc21)c1ccc(-c2ccccc2)c(F)c1]

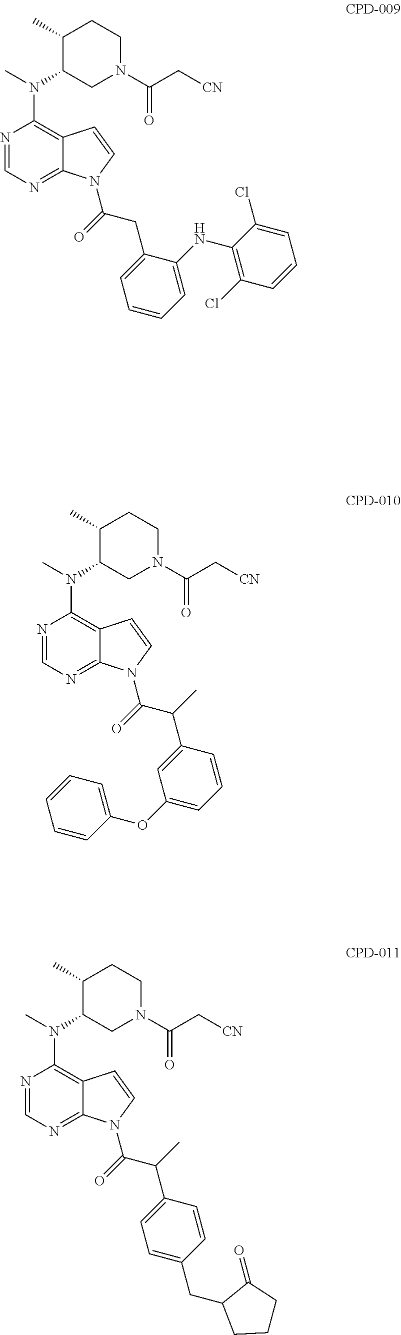 [SMILES: C[C@@H]1CCN(C(=O)CC#N)C[C@@H]1N(C)c1ncnc2c1ccn2C(=O)Cc1ccccc1Nc1c(Cl)cccc1Cl ; CC(C(=O)n1ccc2c(N(C)[C@H]3CN(C(=O)CC#N)CC[C@H]3C)ncnc21)c1cccc(Oc2ccccc2)c1 ; CC(C(=O)n1ccc2c(N(C)[C@H]3CN(C(=O)CC#N)CC[C@H]3C)ncnc21)c1ccc(CC2CCCC2=O)cc1]

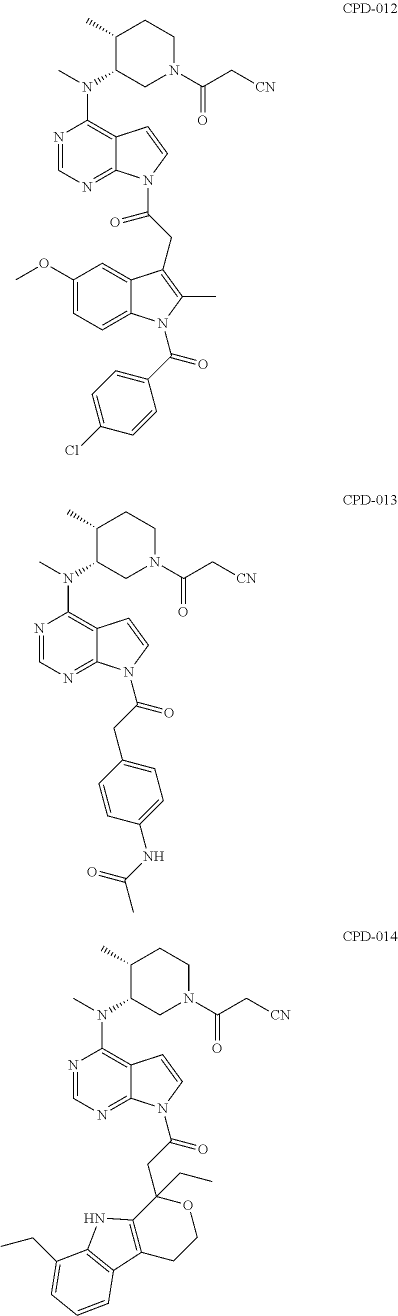

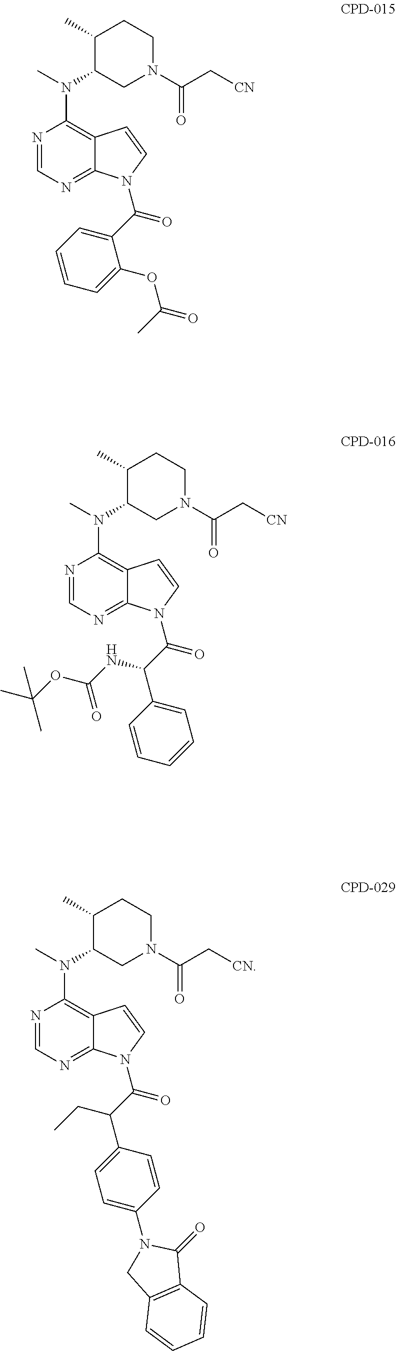 [SMILES: CC(=O)Oc1ccccc1C(=O)n1ccc2c(N(C)[C@H]3CN(C(=O)CC#N)CC[C@H]3C)ncnc21 ; C[C@@H]1CCN(C(=O)CC#N)C[C@@H]1N(C)c1ncnc2c1ccn2C(=O)[C@@H](NC(=O)OC(C)(C)C)c1ccccc1 ; CCC(C(=O)n1ccc2c(N(C)[C@H]3CN(C(=O)CC#N)CC[C@H]3C)ncnc21)c1ccc(N2Cc3ccccc3C2=O)cc1]

Further preferably, the anti-inflammatory compound, or the stereoisomer, tautomer, nitrogen oxide, metabolite, prodrug, pharmaceutically acceptable salt, or solvate thereof of the present invention wherein the compound is one of the following specific compounds:

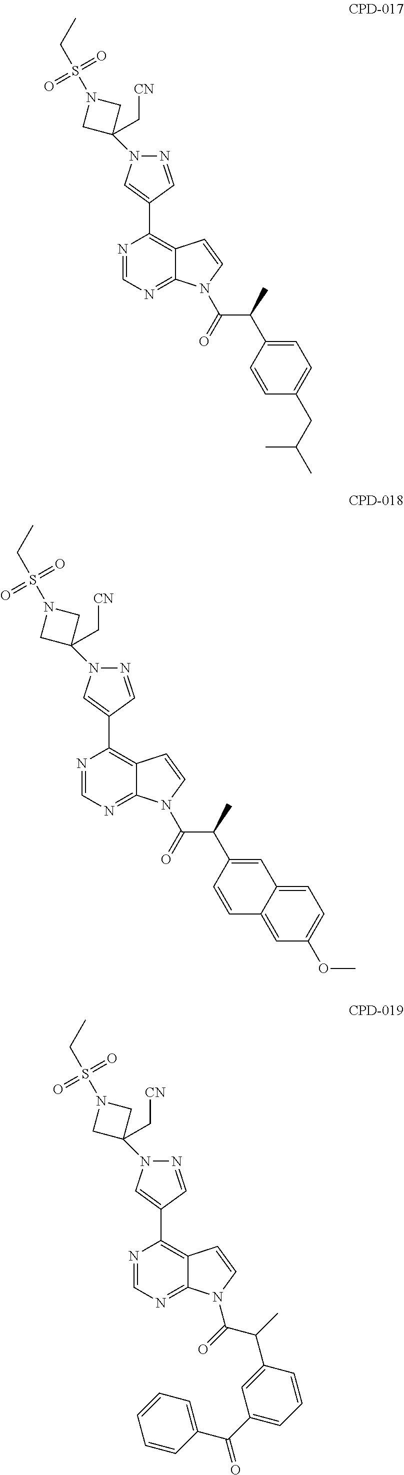 [SMILES: CCS(=O)(=O)N1CC(CC#N)(n2cc(-c3ncnc4c3ccn4C(=O)[C@@H](C)c3ccc(CC(C)C)cc3)cn2)C1 ; CCS(=O)(=O)N1CC(CC#N)(n2cc(-c3ncnc4c3ccn4C(=O)[C@@H](C)c3ccc4cc(OC)ccc4c3)cn2)C1 ; CCS(=O)(=O)N1CC(CC#N)(n2cc(-c3ncnc4c3ccn4C(=O)C(C)c3cccc(C(=O)c4ccccc4)c3)cn2)C1]

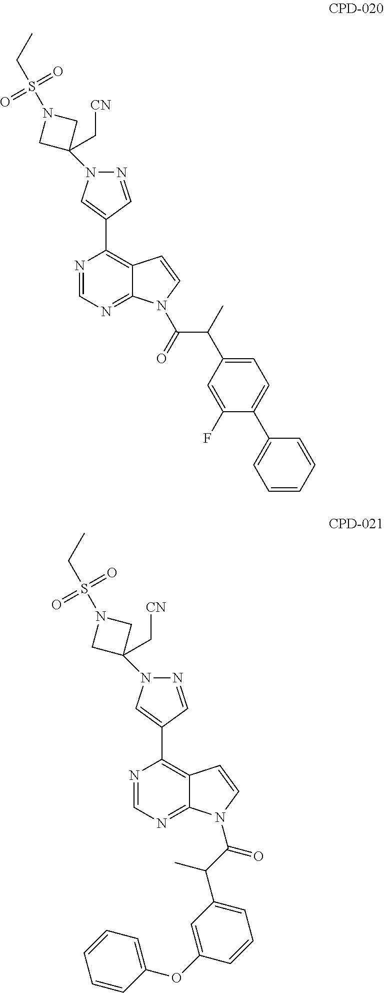 [SMILES: CCS(=O)(=O)N1CC(CC#N)(n2cc(-c3ncnc4c3ccn4C(=O)C(C)c3ccc(-c4ccccc4)c(F)c3)cn2)C1 ; CCS(=O)(=O)N1CC(CC#N)(n2cc(-c3ncnc4c3ccn4C(=O)C(C)c3cccc(Oc4ccccc4)c3)cn2)C1]

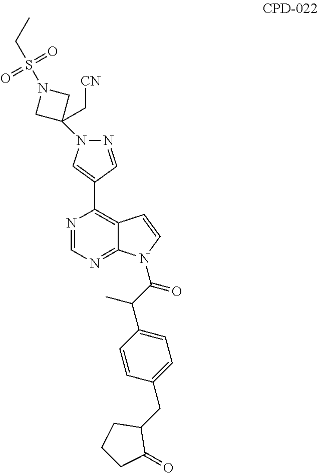 [SMILES: CCS(=O)(=O)N1CC(CC#N)(n2cc(-c3ncnc4c3ccn4C(=O)C(C)c3ccc(CC4CCCC4=O)cc3)cn2)C1]

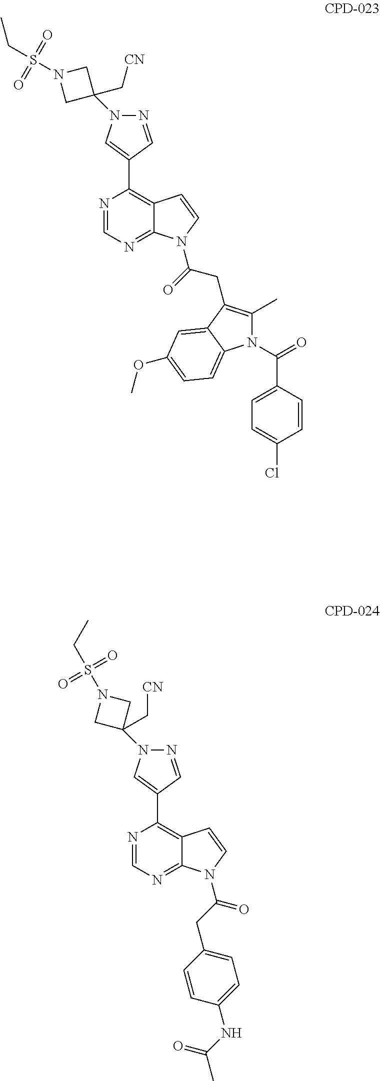 [SMILES: CCS(=O)(=O)N1CC(CC#N)(n2cc(-c3ncnc4c3ccn4C(=O)Cc3c(C)n(C(=O)c4ccc(Cl)cc4)c4ccc(OC)cc34)cn2)C1 ; CCS(=O)(=O)N1CC(CC#N)(n2cc(-c3ncnc4c3ccn4C(=O)Cc3ccc(NC(C)=O)cc3)cn2)C1]

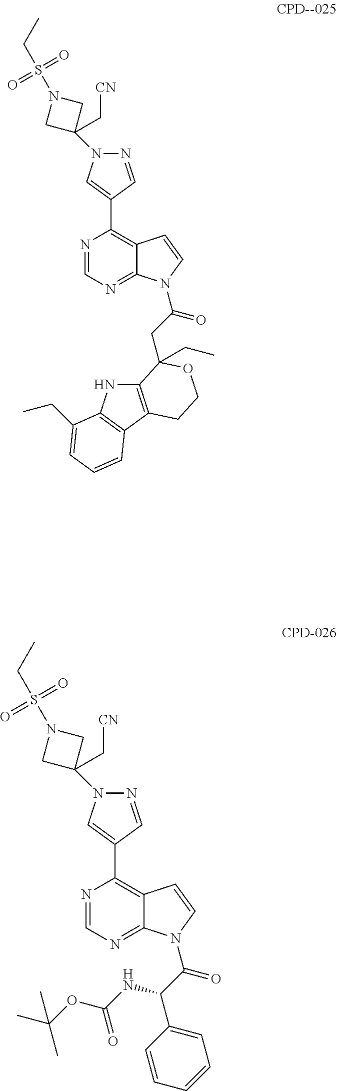 [SMILES: CCc1cccc2c3c([nH]c12)C(CC)(CC(=O)n1ccc2c(-c4cnn(C5(CC#N)CN(S(=O)(=O)CC)C5)c4)ncnc21)OCC3 ; CCS(=O)(=O)N1CC(CC#N)(n2cc(-c3ncnc4c3ccn4C(=O)[C@@H](NC(=O)OC(C)(C)C)c3ccccc3)cn2)C1]

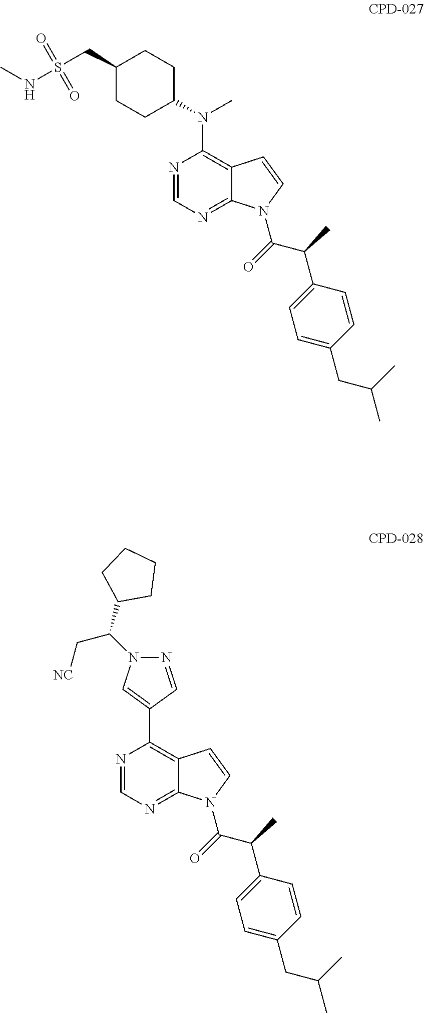

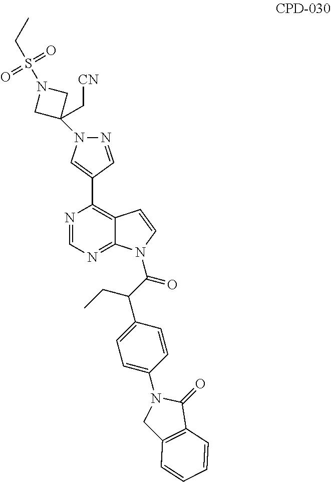 [SMILES: CCC(C(=O)n1ccc2c(-c3cnn(C4(CC#N)CN(S(=O)(=O)CC)C4)c3)ncnc21)c1ccc(N2Cc3ccccc3C2=O)cc1]

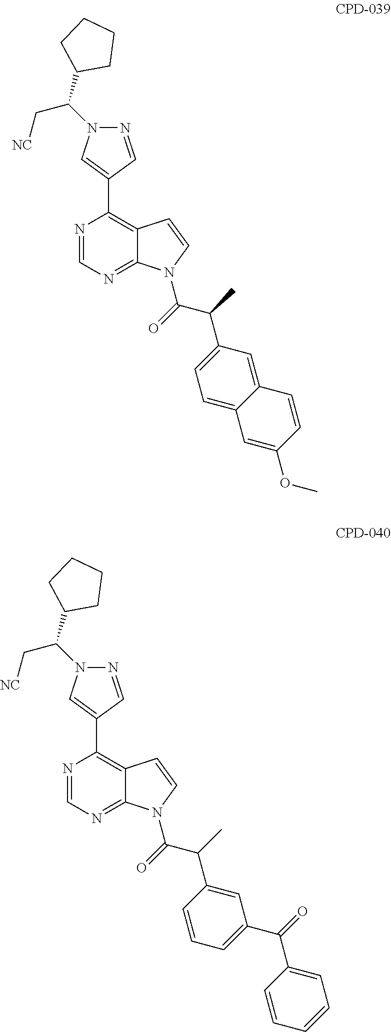 [SMILES: COc1ccc2cc([C@H](C)C(=O)n3ccc4c(-c5cnn([C@H](CC#N)C6CCCC6)c5)ncnc43)ccc2c1 ; CC(C(=O)n1ccc2c(-c3cnn([C@H](CC#N)C4CCCC4)c3)ncnc21)c1cccc(C(=O)c2ccccc2)c1]

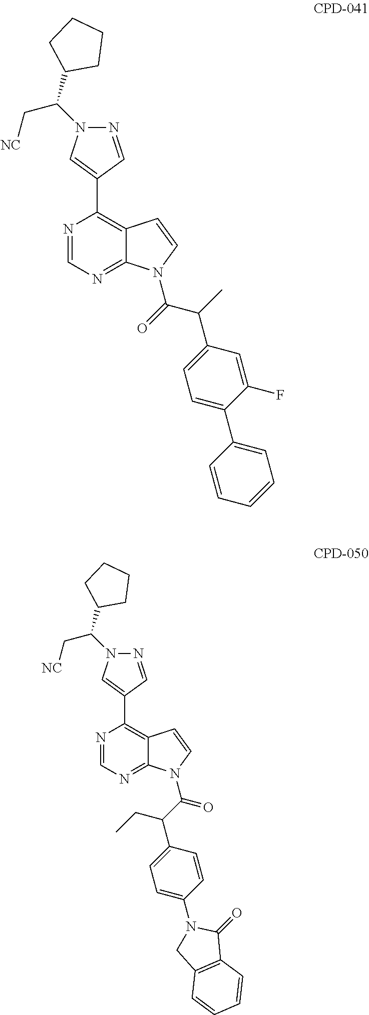 [SMILES: CC(C(=O)n1ccc2c(-c3cnn([C@H](CC#N)C4CCCC4)c3)ncnc21)c1ccc(-c2ccccc2)c(F)c1 ; CCC(C(=O)n1ccc2c(-c3cnn([C@H](CC#N)C4CCCC4)c3)ncnc21)c1ccc(N2Cc3ccccc3C2=O)cc1]

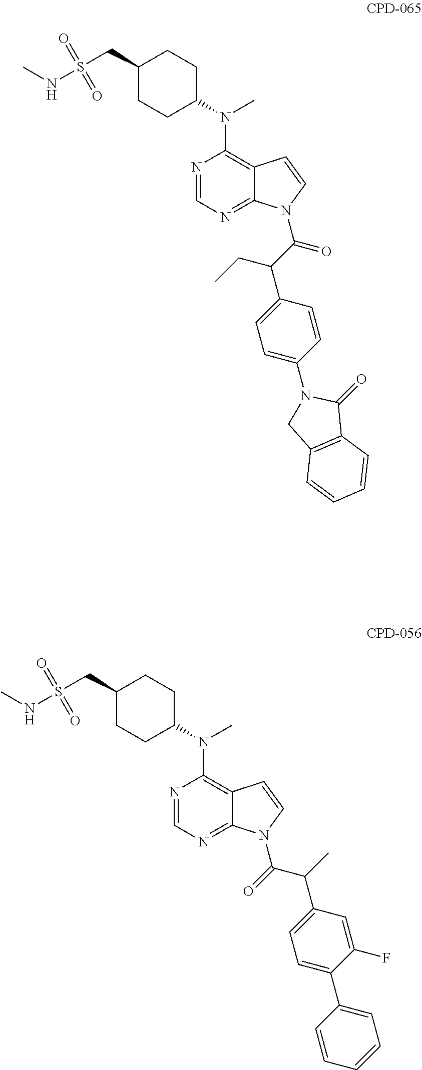

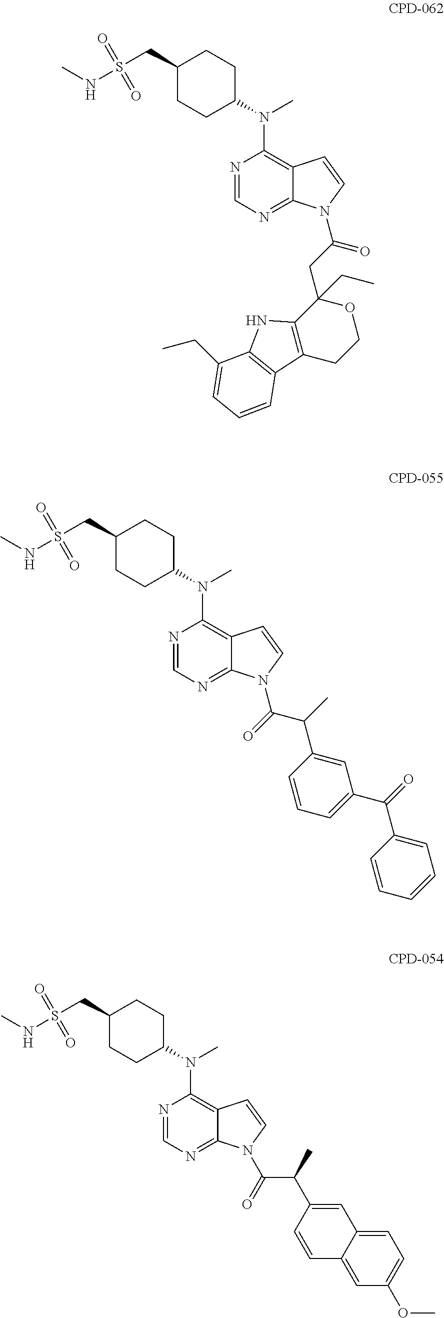

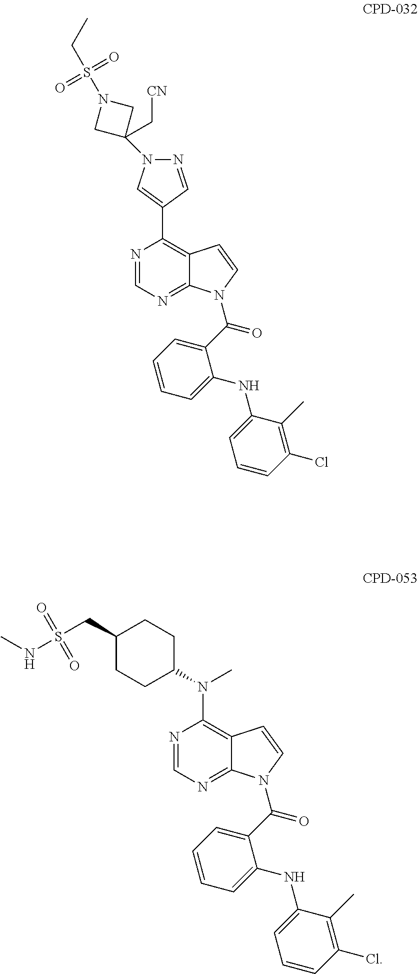

In another scheme of the present invention, the present invention provides an anti-inflammatory compound, or a stereoisomer, tautomer, nitrogen oxide, metabolite, prodrug, pharmaceutically acceptable salt, or solvate thereof, having a structure shown in general formula (III):

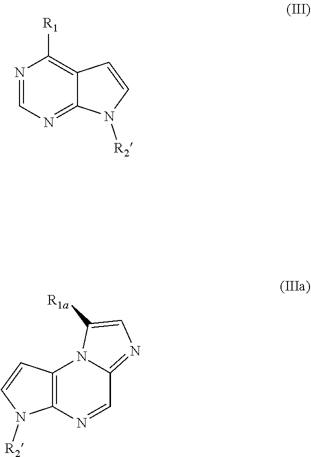

wherein R₁ has the same meaning as R₁ in general formula (II); R₁ₐ has the same meaning as R₁ₐ in formula (IIa);
R₂′ in formula (III) and formula (IIIa) are both Y—B, B is B₁ in formula (II) or (IIa), or B is B₂; wherein the group —B₁ is a group formed by dehydroxylation of a carboxylic acid compound B₁ and Y— is (CH₂)—O—; the group —B₂ is a group formed by dehydrogenation of a hydroxyl-containing compound B₂ and Y— is —(CH₂)—; the group —B₁ has the same meaning as the R₂ group in formula (II) or in formula (IIa); the group —B₂ is R—CO—NH—R_{d}, wherein R_{c} is a 4-hydroxy-benzothiazine dioxide-3-yl represented by the following structural formula (a)(wherein the phenyl ring may be substituted by halogen or C₁-C₆ alkyl), or a 4-hydroxy-Re substituted thienothiazine dioxide-3-yl represented by the following structural formula (b), wherein —CO—NH—R_{d} is bonded at the 3-position of the thiazine ring,

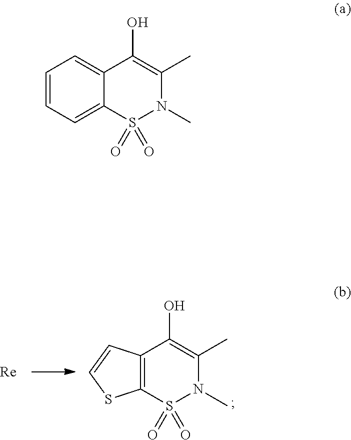

wherein R_{d} is thiazole, isothiazole, oxazole, isoxazole, or pyridine or the group thereof substituted with C₁-C₆ alkyl or halogen, preferably a thiazole or isoxazole substituted with methyl; and unsubstituted pyridyl; Rₑ is C₁-C₆ alkyl or halogen (preferably halogen is one or two or more selected from a group consisting of fluorine, chlorine, or bromine); a arrow next to Rₑ in formula (b) indicates that its substitution position on the thiophene ring may be any carbon-linked hydrogen atom capable of undergoing substitution.

More specifically, the anti-inflammatory compound or a stereoisomer, tautomer, nitrogen oxide, metabolite, prodrug, pharmaceutically acceptable salt, or solvate thereof of the present invention, wherein —B₁ is a group after dehydroxylation of a carboxylic acid moiety selected from a group consisting of ibuprofen, (S)-(+)-ibuprofen, naproxen, fenoprofen, flurbiprofen, loxoprofen, ketoprofen, diclofenac, etodolac, actarit, indomethacin, N-Boc-L-phenylglycine, aspirin, indobufen, mefenamic acid and tolfenamic acid:

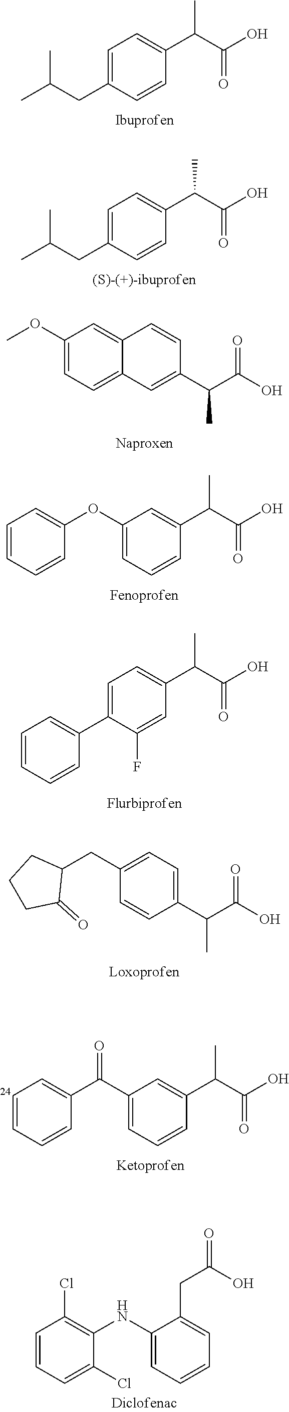

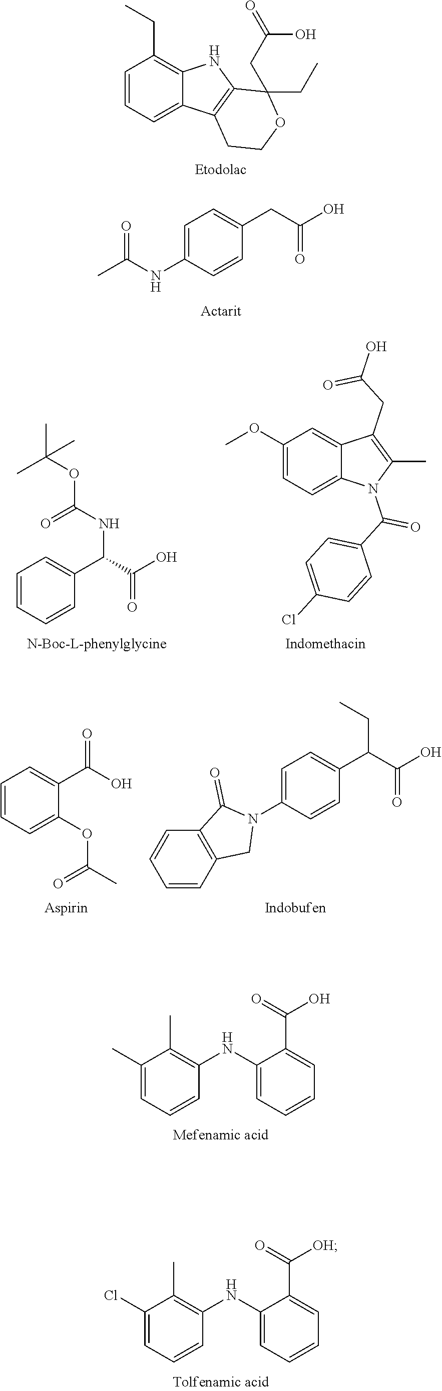

—B₂ is a group after dehydrogenation of a hydroxyl-containing compound of one of the following specific compounds:

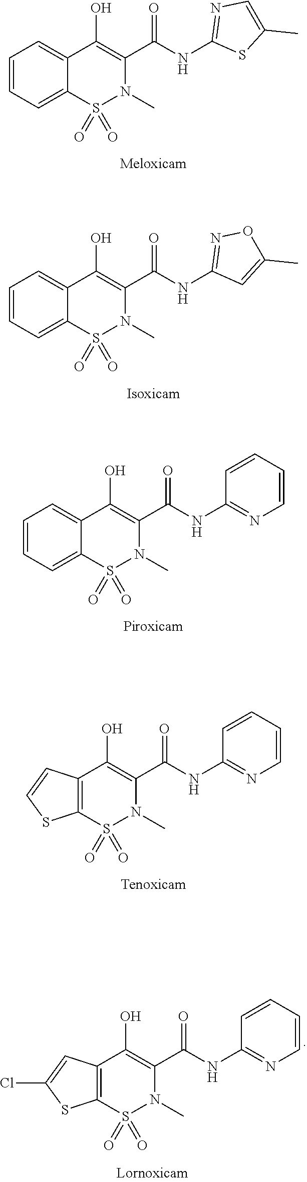 [SMILES: Cc1cnc(NC(=O)C2=C(O)c3ccccc3S(=O)(=O)N2C)s1 ; Cc1cc(NC(=O)C2=C(O)c3ccccc3S(=O)(=O)N2C)no1 ; CN1C(C(=O)Nc2ccccn2)=C(O)c2ccccc2S1(=O)=O ; CN1C(C(=O)Nc2ccccn2)=C(O)c2ccsc2S1(=O)=O ; CN1C(C(=O)Nc2ccccn2)=C(O)c2cc(Cl)sc2S1(=O)=O]

Furthermore, more specifically, the anti-inflammatory compound, or the stereoisomer, tautomer, nitrogen oxide, metabolite, prodrug, pharmaceutically acceptable salt, or solvate thereof of the present invention, obtained by a preparation method comprising the steps of
2) reacting A-CH₂—OH compound with an acyl chloride of B or directly with the B compound;
Wherein the A-CH₂—OH compound is preferably prepared by the following step 1): reacting the amine compound A to form the A-CH₂—OH compound.

More specifically, the anti-inflammatory compound, or a stereoisomer, tautomer, nitrogen oxide, metabolite, prodrug, pharmaceutically acceptable salt, or solvate thereof described above, wherein A- is a group after dehydrogenation of an amine compound selected from a group consisting of any one of the following groups: tofacitinib, baricitinib, oclacitinib, ruxolitinib, upadacitinib and delgocitinib:

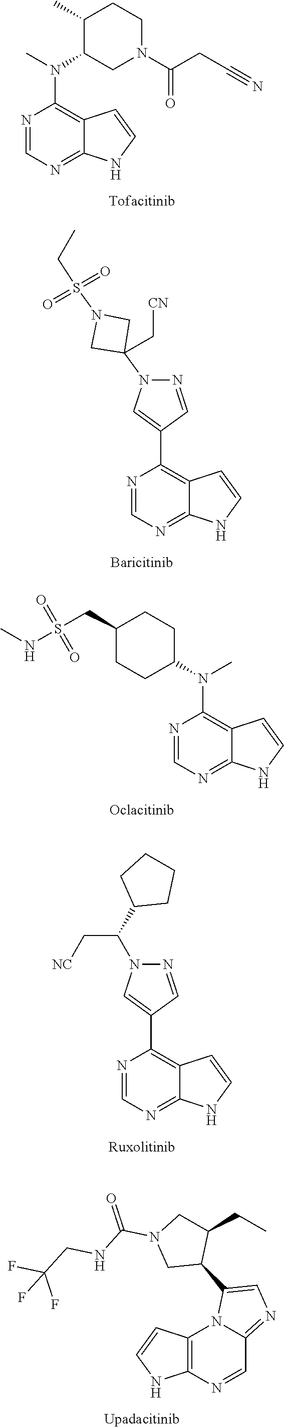

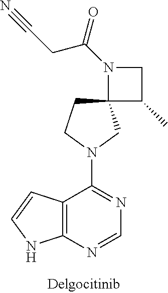 [SMILES: C[C@H]1CN(C(=O)CC#N)[C@]12CCN(c1ncnc3[nH]ccc13)C2]

preferably A is a group formed after dehydrogenation of tofacitinib, ruxolitinib, and baricitinib.

In certain embodiments, A is a group formed by any one of baricitinib, oclacitinib, or upadacitinib.

Further, more specifically, the anti-inflammatory compound, or the stereoisomer, tautomer, nitrogen oxide, metabolite, prodrug, pharmaceutically acceptable salt, or solvate thereof described above, wherein the compound is any one of the following specific compounds:

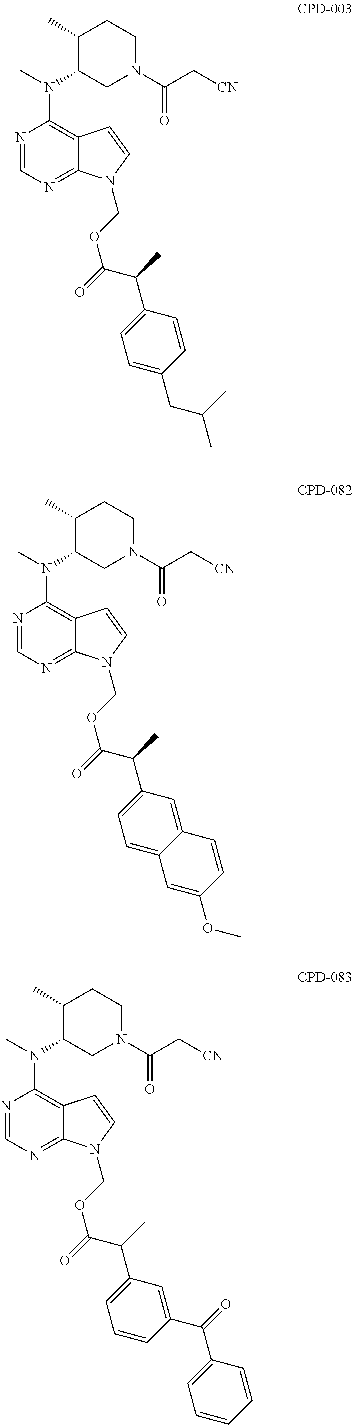 [SMILES: CC(C)Cc1ccc([C@H](C)C(=O)OCn2ccc3c(N(C)[C@H]4CN(C(=O)CC#N)CC[C@H]4C)ncnc32)cc1 ; COc1ccc2cc([C@H](C)C(=O)OCn3ccc4c(N(C)[C@H]5CN(C(=O)CC#N)CC[C@H]5C)ncnc43)ccc2c1 ; CC(C(=O)OCn1ccc2c(N(C)[C@H]3CN(C(=O)CC#N)CC[C@H]3C)ncnc21)c1cccc(C(=O)c2ccccc2)c1]

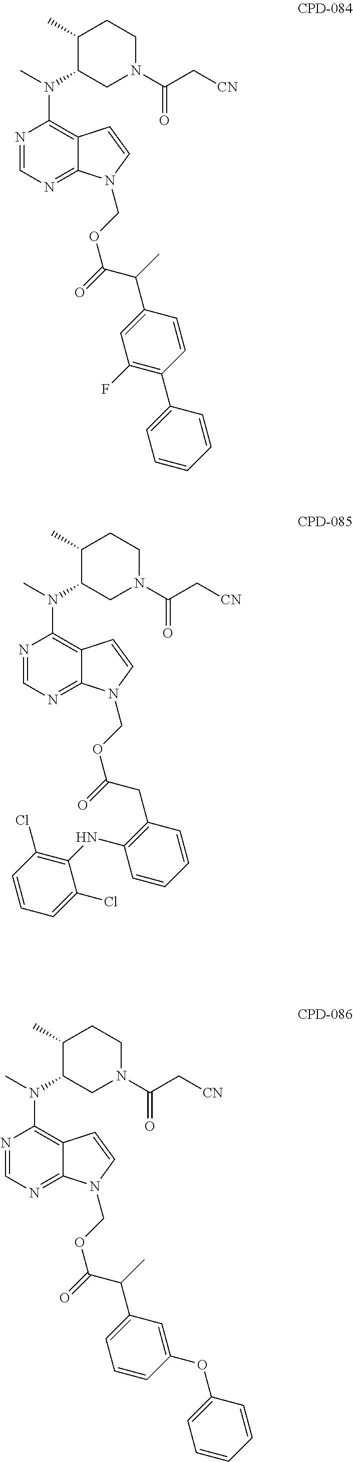 [SMILES: CC(C(=O)OCn1ccc2c(N(C)[C@H]3CN(C(=O)CC#N)CC[C@H]3C)ncnc21)c1ccc(-c2ccccc2)c(F)c1 ; C[C@@H]1CCN(C(=O)CC#N)C[C@@H]1N(C)c1ncnc2c1ccn2COC(=O)Cc1ccccc1Nc1c(Cl)cccc1Cl ; CC(C(=O)OCn1ccc2c(N(C)[C@H]3CN(C(=O)CC#N)CC[C@H]3C)ncnc21)c1cccc(Oc2ccccc2)c1]

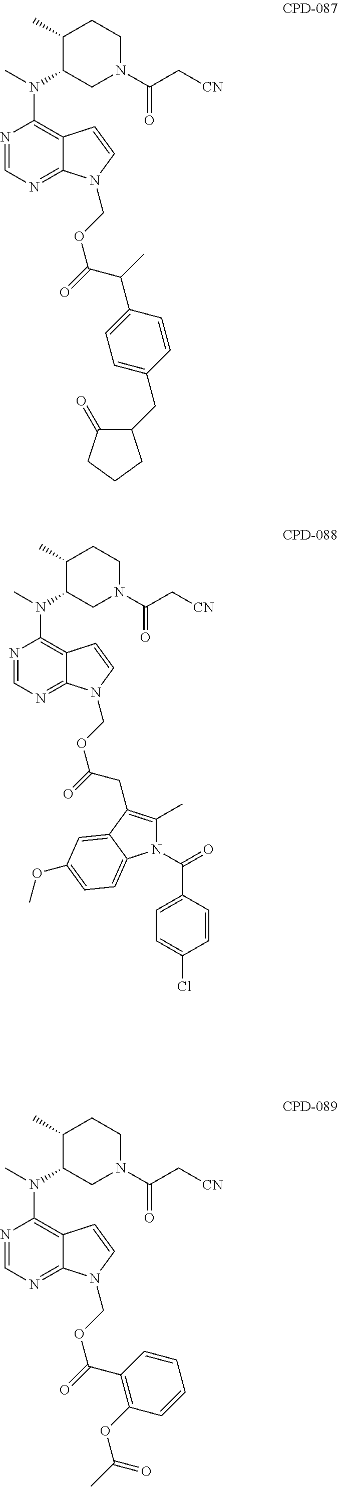 [SMILES: CC(C(=O)OCn1ccc2c(N(C)[C@H]3CN(C(=O)CC#N)CC[C@H]3C)ncnc21)c1ccc(CC2CCCC2=O)cc1 ; COc1ccc2c(c1)c(CC(=O)OCn1ccc3c(N(C)[C@H]4CN(C(=O)CC#N)CC[C@H]4C)ncnc31)c(C)n2C(=O)c1ccc(Cl)cc1 ; CC(=O)Oc1ccccc1C(=O)OCn1ccc2c(N(C)[C@H]3CN(C(=O)CC#N)CC[C@H]3C)ncnc21]

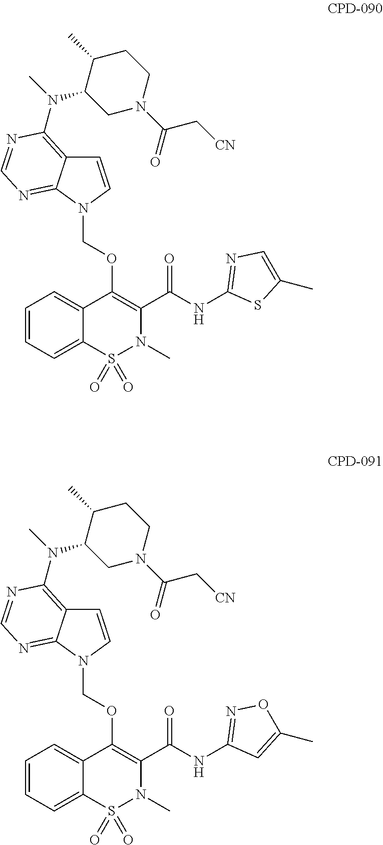 [SMILES: Cc1cnc(NC(=O)C2=C(OCn3ccc4c(N(C)[C@H]5CN(C(=O)CC#N)CC[C@H]5C)ncnc43)c3ccccc3S(=O)(=O)N2C)s1 ; Cc1cc(NC(=O)C2=C(OCn3ccc4c(N(C)[C@H]5CN(C(=O)CC#N)CC[C@H]5C)ncnc43)c3ccccc3S(=O)(=O)N2C)no1]

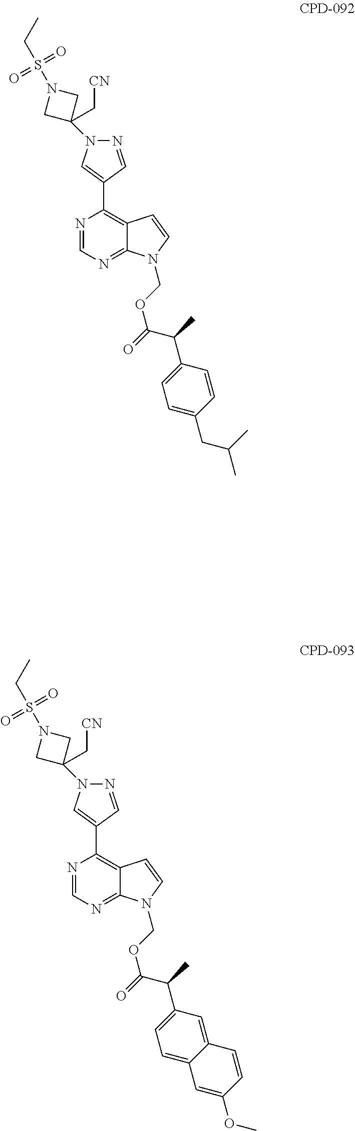 [SMILES: CCS(=O)(=O)N1CC(CC#N)(n2cc(-c3ncnc4c3ccn4COC(=O)[C@@H](C)c3ccc(CC(C)C)cc3)cn2)C1 ; CCS(=O)(=O)N1CC(CC#N)(n2cc(-c3ncnc4c3ccn4COC(=O)[C@@H](C)c3ccc4cc(OC)ccc4c3)cn2)C1]

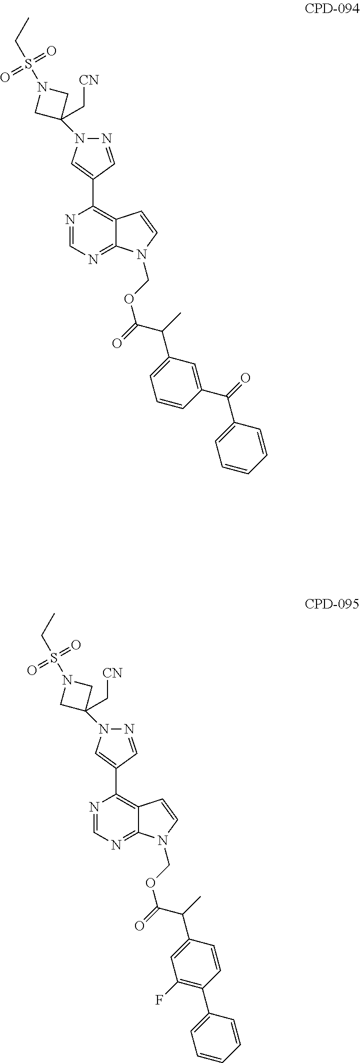 [SMILES: CCS(=O)(=O)N1CC(CC#N)(n2cc(-c3ncnc4c3ccn4COC(=O)C(C)c3cccc(C(=O)c4ccccc4)c3)cn2)C1 ; CCS(=O)(=O)N1CC(CC#N)(n2cc(-c3ncnc4c3ccn4COC(=O)C(C)c3ccc(-c4ccccc4)c(F)c3)cn2)C1]

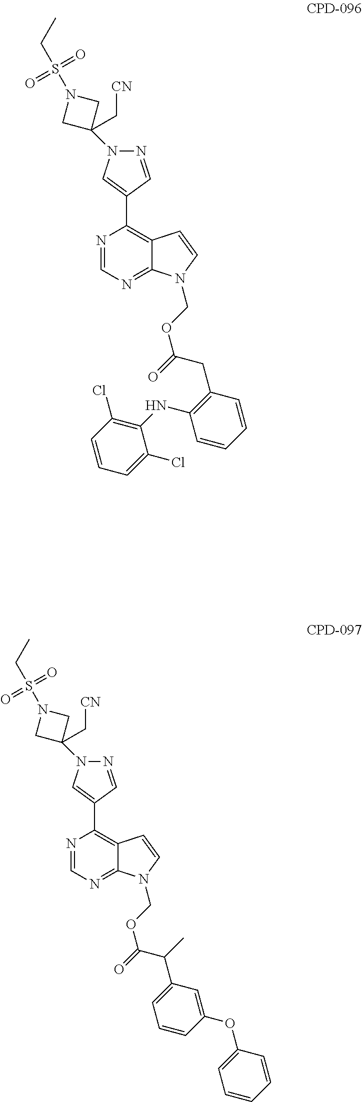 [SMILES: CCS(=O)(=O)N1CC(CC#N)(n2cc(-c3ncnc4c3ccn4COC(=O)Cc3ccccc3Nc3c(Cl)cccc3Cl)cn2)C1 ; CCS(=O)(=O)N1CC(CC#N)(n2cc(-c3ncnc4c3ccn4COC(=O)C(C)c3cccc(Oc4ccccc4)c3)cn2)C1]

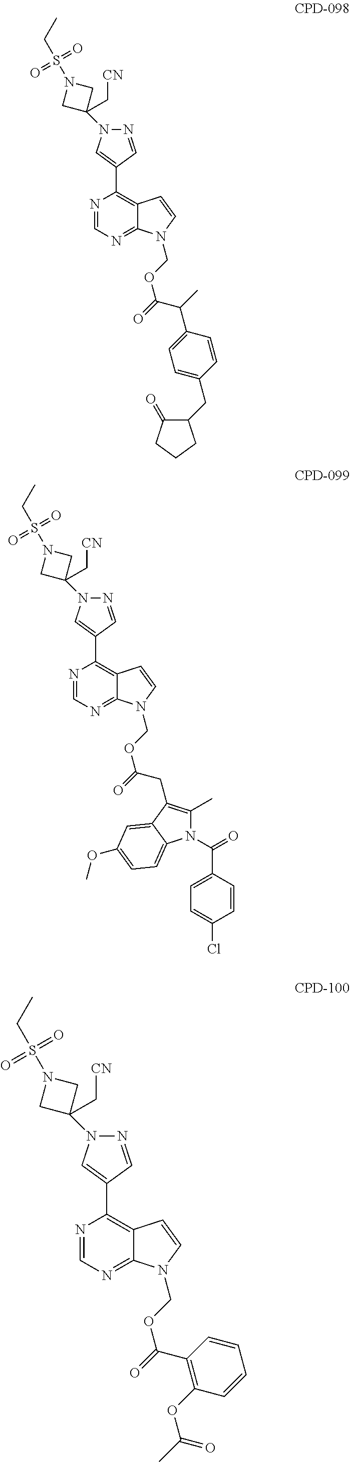 [SMILES: CCS(=O)(=O)N1CC(CC#N)(n2cc(-c3ncnc4c3ccn4COC(=O)C(C)c3ccc(CC4CCCC4=O)cc3)cn2)C1 ; CCS(=O)(=O)N1CC(CC#N)(n2cc(-c3ncnc4c3ccn4COC(=O)Cc3c(C)n(C(=O)c4ccc(Cl)cc4)c4ccc(OC)cc34)cn2)C1 ; CCS(=O)(=O)N1CC(CC#N)(n2cc(-c3ncnc4c3ccn4COC(=O)c3ccccc3OC(C)=O)cn2)C1]

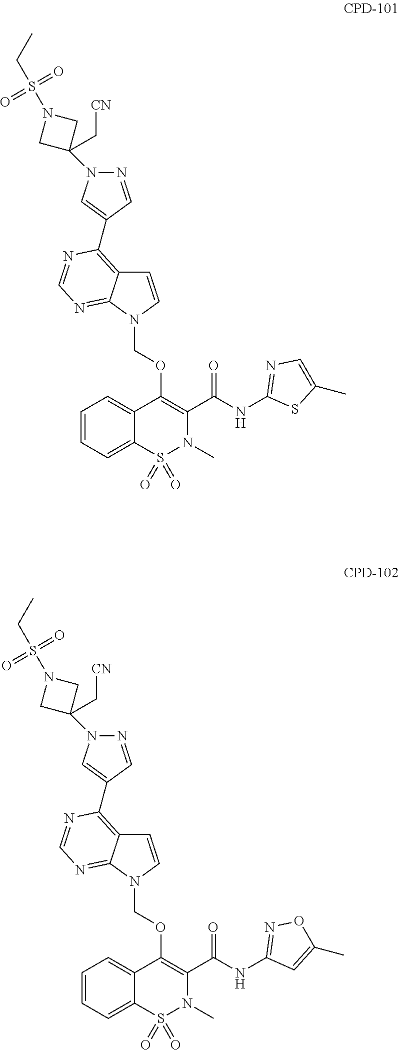 [SMILES: CCS(=O)(=O)N1CC(CC#N)(n2cc(-c3ncnc4c3ccn4COC3=C(C(=O)Nc4ncc(C)s4)N(C)S(=O)(=O)c4ccccc43)cn2)C1 ; CCS(=O)(=O)N1CC(CC#N)(n2cc(-c3ncnc4c3ccn4COC3=C(C(=O)Nc4cc(C)on4)N(C)S(=O)(=O)c4ccccc43)cn2)C1]

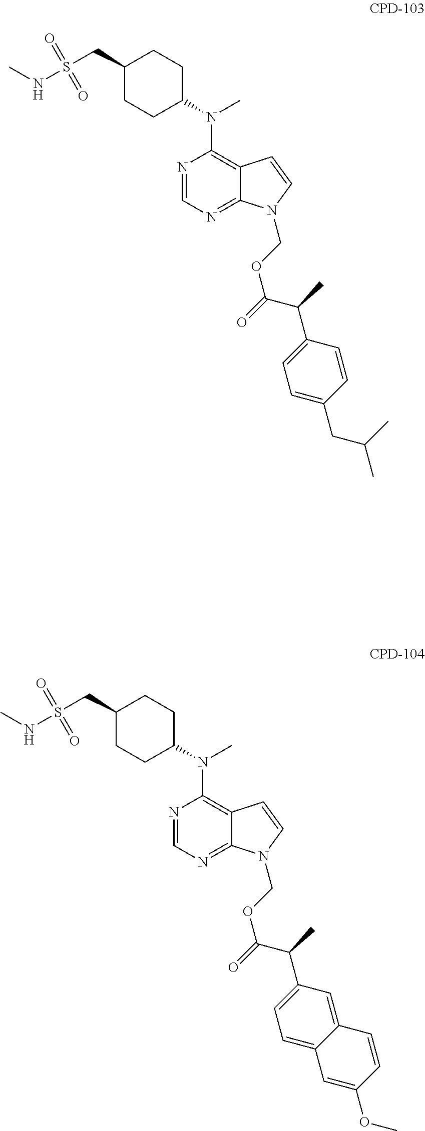

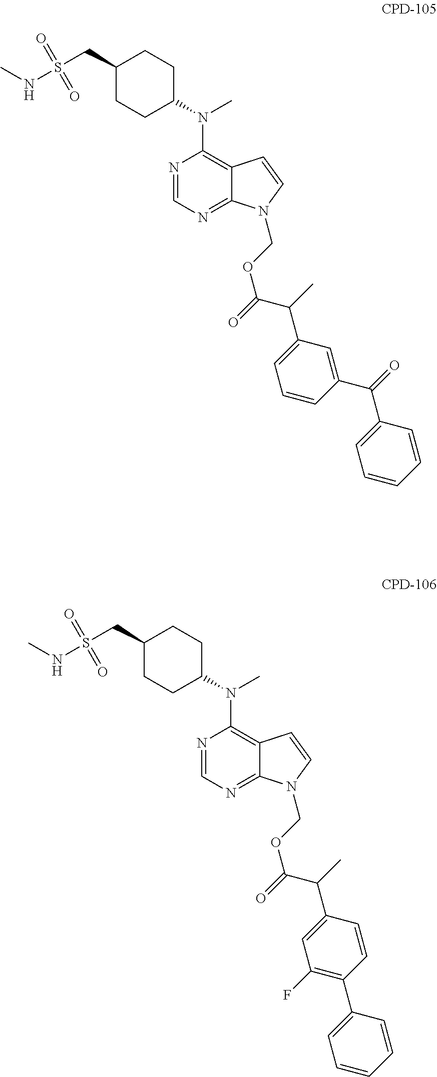

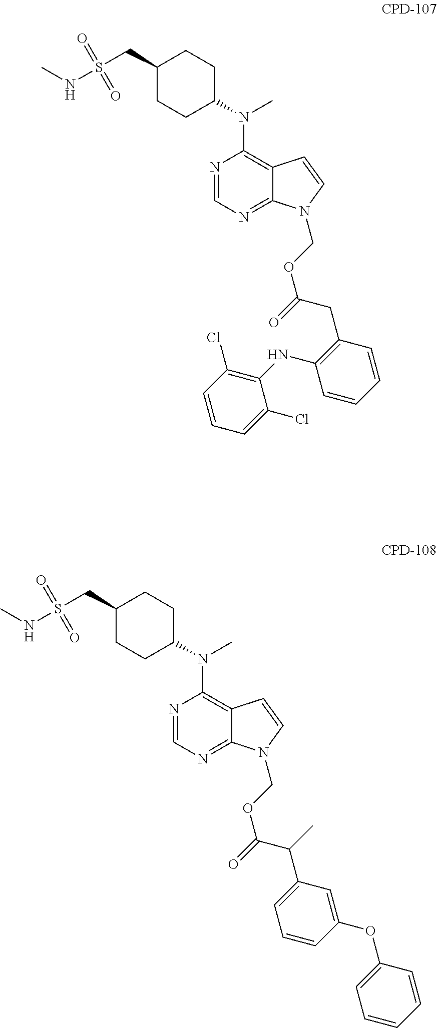

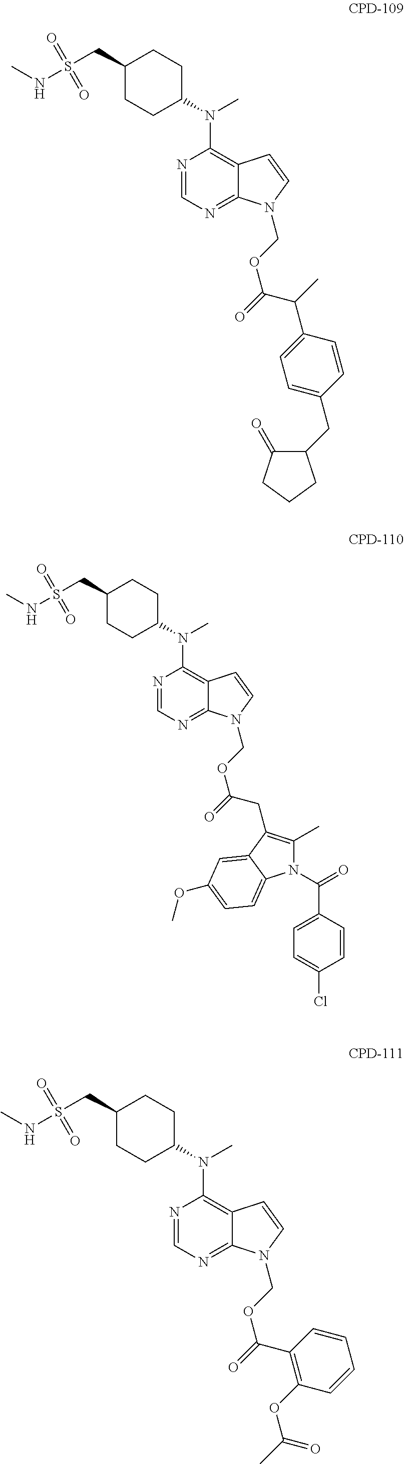

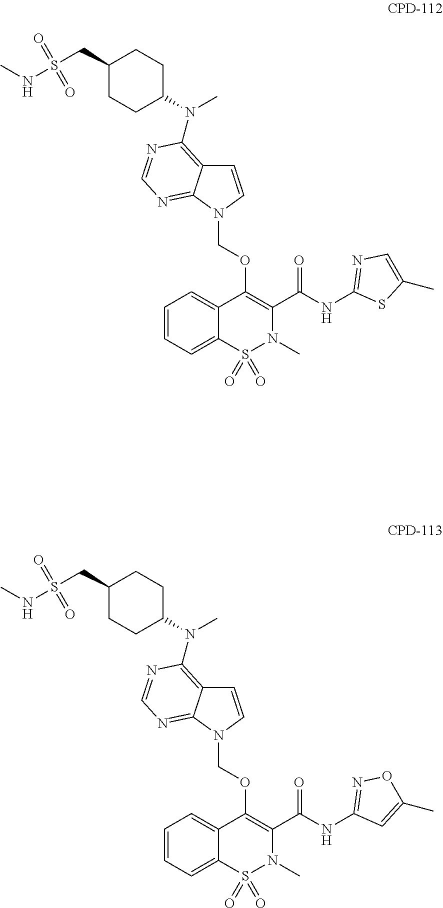

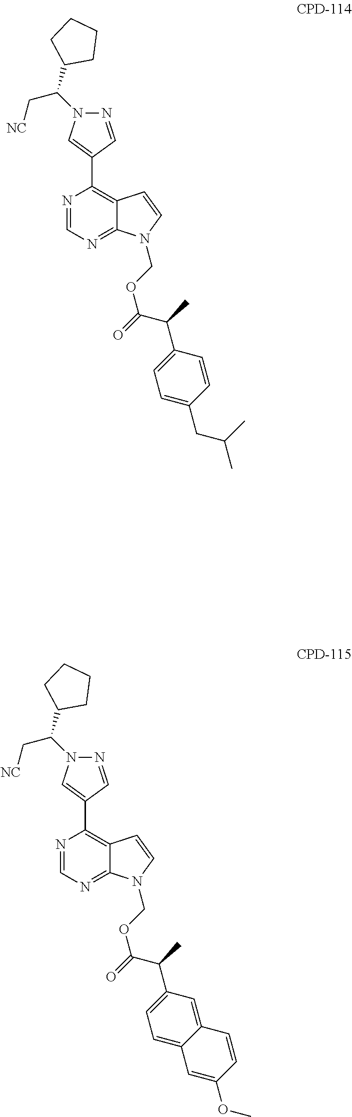 [SMILES: CC(C)Cc1ccc([C@H](C)C(=O)OCn2ccc3c(-c4cnn([C@H](CC#N)C5CCCC5)c4)ncnc32)cc1 ; COc1ccc2cc([C@H](C)C(=O)OCn3ccc4c(-c5cnn([C@H](CC#N)C6CCCC6)c5)ncnc43)ccc2c1]

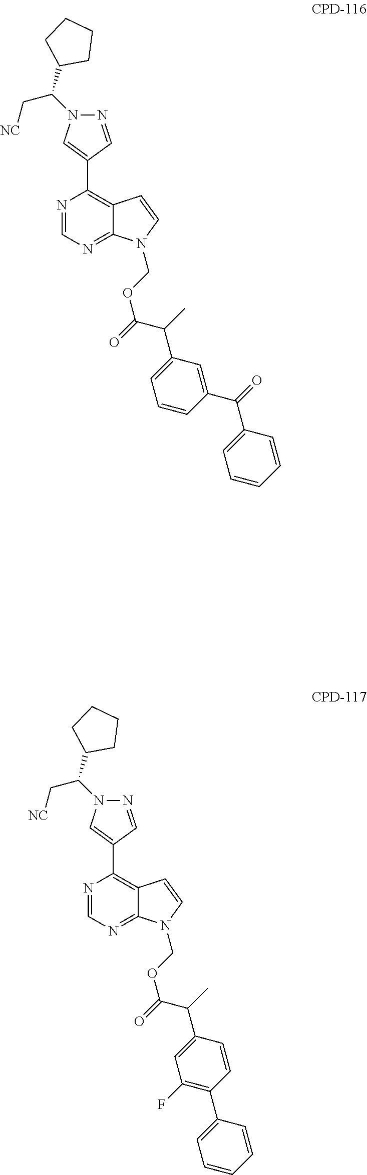 [SMILES: CC(C(=O)OCn1ccc2c(-c3cnn([C@H](CC#N)C4CCCC4)c3)ncnc21)c1cccc(C(=O)c2ccccc2)c1 ; CC(C(=O)OCn1ccc2c(-c3cnn([C@H](CC#N)C4CCCC4)c3)ncnc21)c1ccc(-c2ccccc2)c(F)c1]

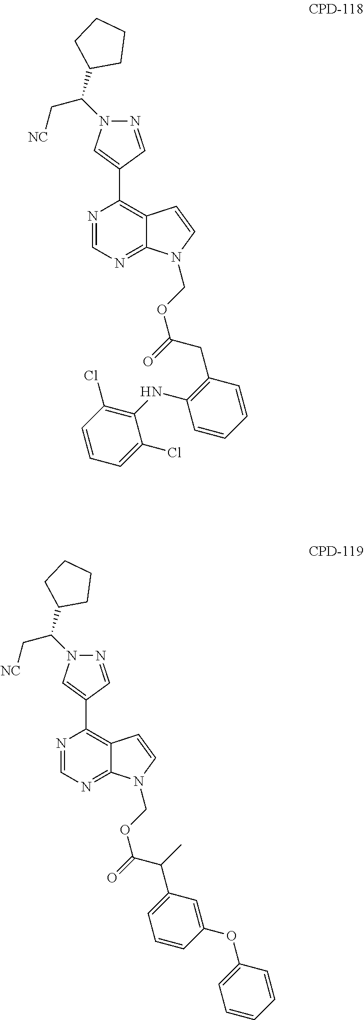 [SMILES: N#CC[C@H](C1CCCC1)n1cc(-c2ncnc3c2ccn3COC(=O)Cc2ccccc2Nc2c(Cl)cccc2Cl)cn1 ; CC(C(=O)OCn1ccc2c(-c3cnn([C@H](CC#N)C4CCCC4)c3)ncnc21)c1cccc(Oc2ccccc2)c1]

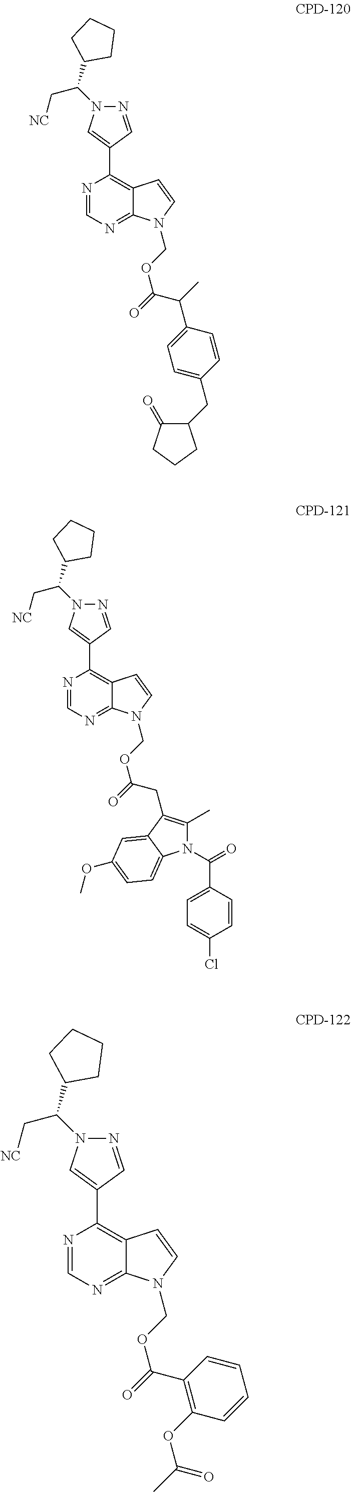 [SMILES: CC(C(=O)OCn1ccc2c(-c3cnn([C@H](CC#N)C4CCCC4)c3)ncnc21)c1ccc(CC2CCCC2=O)cc1 ; COc1ccc2c(c1)c(CC(=O)OCn1ccc3c(-c4cnn([C@H](CC#N)C5CCCC5)c4)ncnc31)c(C)n2C(=O)c1ccc(Cl)cc1 ; CC(=O)Oc1ccccc1C(=O)OCn1ccc2c(-c3cnn([C@H](CC#N)C4CCCC4)c3)ncnc21]

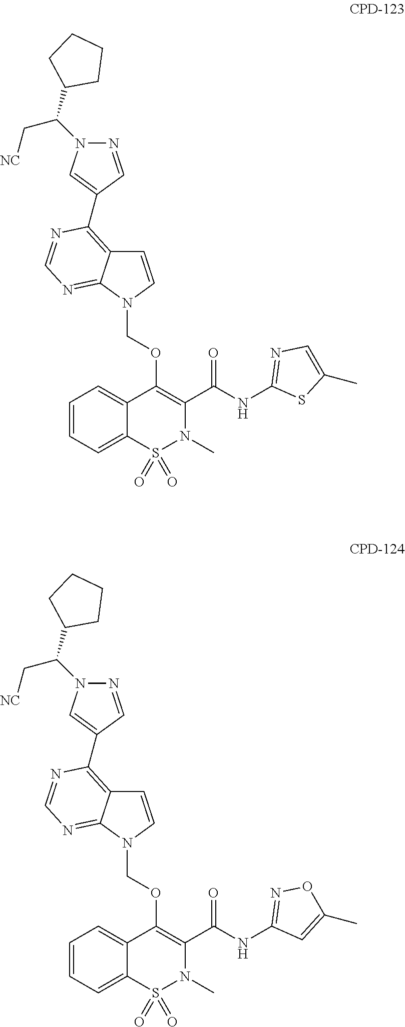 [SMILES: Cc1cnc(NC(=O)C2=C(OCn3ccc4c(-c5cnn([C@H](CC#N)C6CCCC6)c5)ncnc43)c3ccccc3S(=O)(=O)N2C)s1 ; Cc1cc(NC(=O)C2=C(OCn3ccc4c(-c5cnn([C@H](CC#N)C6CCCC6)c5)ncnc43)c3ccccc3S(=O)(=O)N2C)no1]

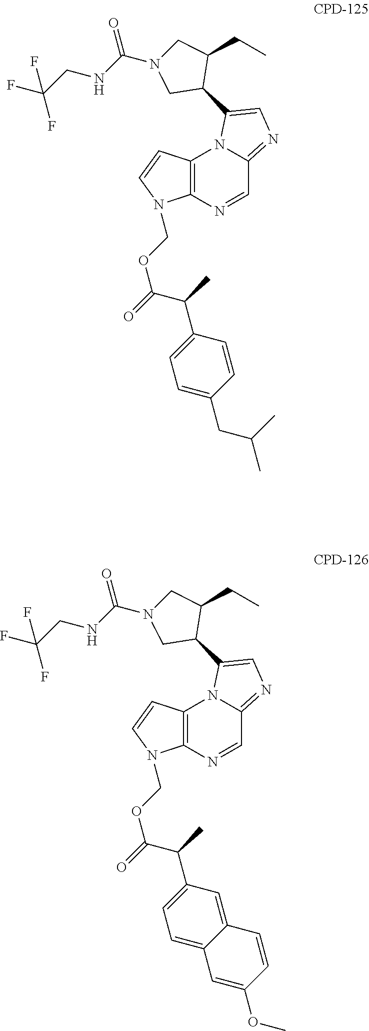 [SMILES: CC[C@@H]1CN(C(=O)NCC(F)(F)F)C[C@@H]1c1cnc2cnc3c(ccn3COC(=O)[C@@H](C)c3ccc(CC(C)C)cc3)n12 ; CC[C@@H]1CN(C(=O)NCC(F)(F)F)C[C@@H]1c1cnc2cnc3c(ccn3COC(=O)[C@@H](C)c3ccc4cc(OC)ccc4c3)n12]

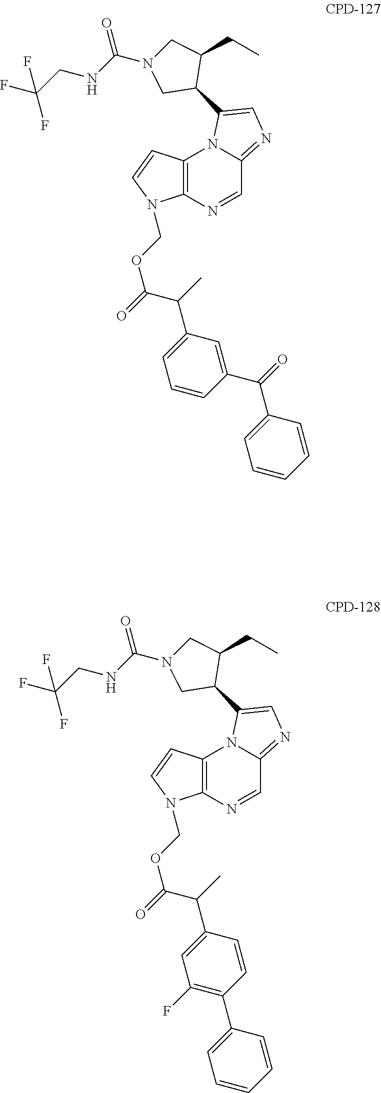 [SMILES: CC[C@@H]1CN(C(=O)NCC(F)(F)F)C[C@@H]1c1cnc2cnc3c(ccn3COC(=O)C(C)c3cccc(C(=O)c4ccccc4)c3)n12 ; CC[C@@H]1CN(C(=O)NCC(F)(F)F)C[C@@H]1c1cnc2cnc3c(ccn3COC(=O)C(C)c3ccc(-c4ccccc4)c(F)c3)n12]

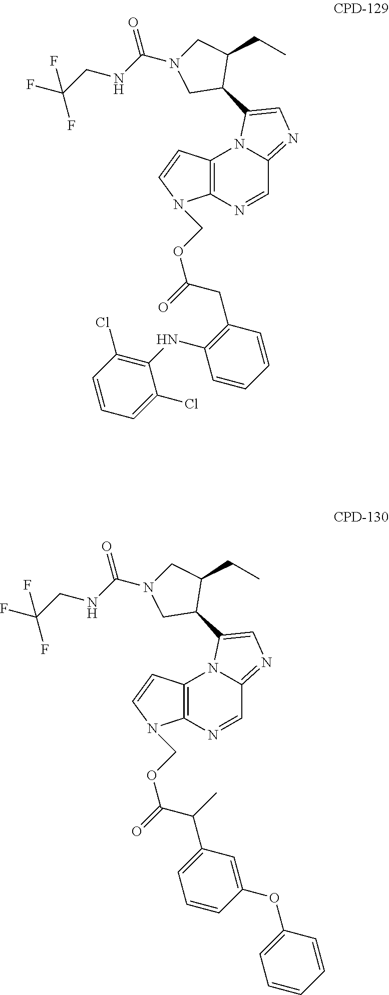 [SMILES: CC[C@@H]1CN(C(=O)NCC(F)(F)F)C[C@@H]1c1cnc2cnc3c(ccn3COC(=O)Cc3ccccc3Nc3c(Cl)cccc3Cl)n12 ; CC[C@@H]1CN(C(=O)NCC(F)(F)F)C[C@@H]1c1cnc2cnc3c(ccn3COC(=O)C(C)c3cccc(Oc4ccccc4)c3)n12]

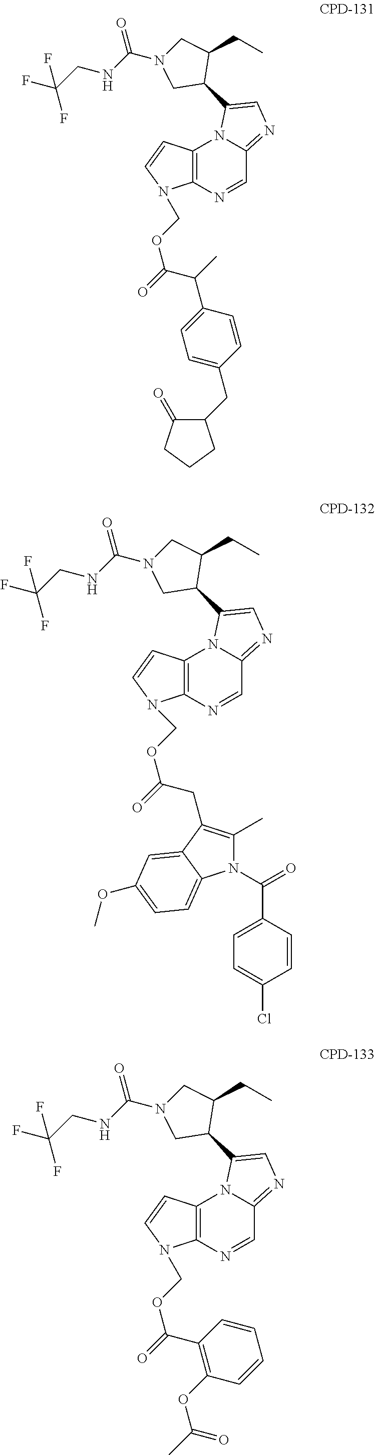 [SMILES: CC[C@@H]1CN(C(=O)NCC(F)(F)F)C[C@@H]1c1cnc2cnc3c(ccn3COC(=O)C(C)c3ccc(CC4CCCC4=O)cc3)n12 ; CC[C@@H]1CN(C(=O)NCC(F)(F)F)C[C@@H]1c1cnc2cnc3c(ccn3COC(=O)Cc3c(C)n(C(=O)c4ccc(Cl)cc4)c4ccc(OC)cc34)n12 ; CC[C@@H]1CN(C(=O)NCC(F)(F)F)C[C@@H]1c1cnc2cnc3c(ccn3COC(=O)c3ccccc3OC(C)=O)n12]

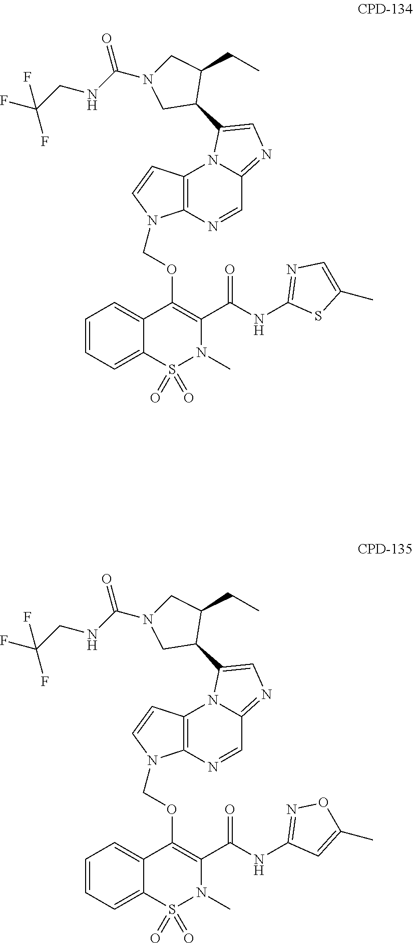 [SMILES: CC[C@@H]1CN(C(=O)NCC(F)(F)F)C[C@@H]1c1cnc2cnc3c(ccn3COC3=C(C(=O)Nc4ncc(C)s4)N(C)S(=O)(=O)c4ccccc43)n12 ; CC[C@@H]1CN(C(=O)NCC(F)(F)F)C[C@@H]1c1cnc2cnc3c(ccn3COC3=C(C(=O)Nc4cc(C)on4)N(C)S(=O)(=O)c4ccccc43)n12]

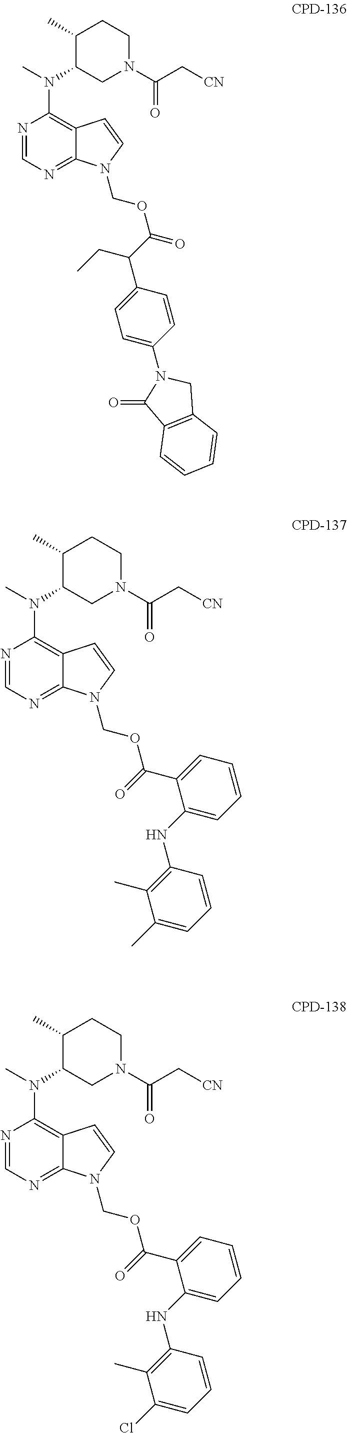 [SMILES: CCC(C(=O)OCn1ccc2c(N(C)[C@H]3CN(C(=O)CC#N)CC[C@H]3C)ncnc21)c1ccc(N2Cc3ccccc3C2=O)cc1 ; Cc1cccc(Nc2ccccc2C(=O)OCn2ccc3c(N(C)[C@H]4CN(C(=O)CC#N)CC[C@H]4C)ncnc32)c1C ; Cc1c(Cl)cccc1Nc1ccccc1C(=O)OCn1ccc2c(N(C)[C@H]3CN(C(=O)CC#N)CC[C@H]3C)ncnc21]

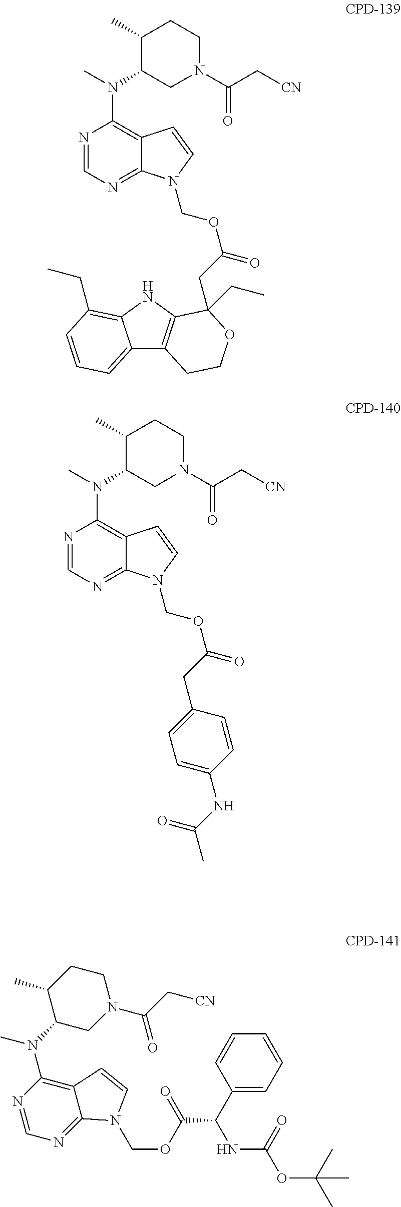 [SMILES: CCc1cccc2c3c([nH]c12)C(CC)(CC(=O)OCn1ccc2c(N(C)[C@H]4CN(C(=O)CC#N)CC[C@H]4C)ncnc21)OCC3 ; CC(=O)Nc1ccc(CC(=O)OCn2ccc3c(N(C)[C@H]4CN(C(=O)CC#N)CC[C@H]4C)ncnc32)cc1 ; C[C@@H]1CCN(C(=O)CC#N)C[C@@H]1N(C)c1ncnc2c1ccn2COC(=O)[C@@H](NC(=O)OC(C)(C)C)c1ccccc1]

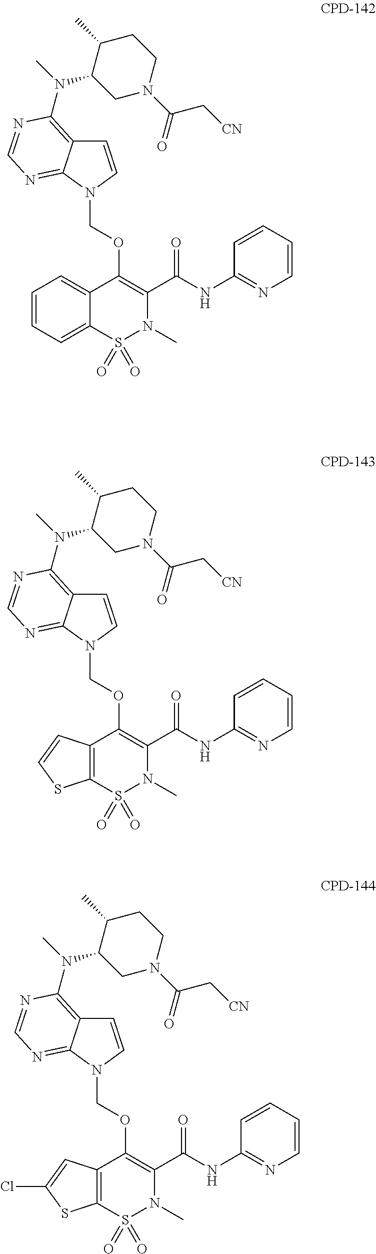 [SMILES: C[C@@H]1CCN(C(=O)CC#N)C[C@@H]1N(C)c1ncnc2c1ccn2COC1=C(C(=O)Nc2ccccn2)N(C)S(=O)(=O)c2ccccc21 ; C[C@@H]1CCN(C(=O)CC#N)C[C@@H]1N(C)c1ncnc2c1ccn2COC1=C(C(=O)Nc2ccccn2)N(C)S(=O)(=O)c2sccc21 ; C[C@@H]1CCN(C(=O)CC#N)C[C@@H]1N(C)c1ncnc2c1ccn2COC1=C(C(=O)Nc2ccccn2)N(C)S(=O)(=O)c2sc(Cl)cc21]

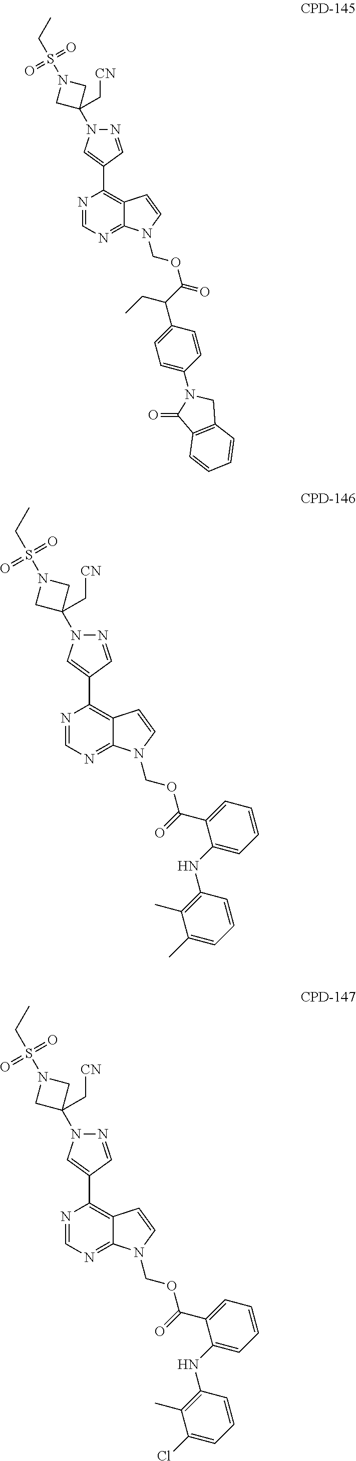 [SMILES: CCC(C(=O)OCn1ccc2c(-c3cnn(C4(CC#N)CN(S(=O)(=O)CC)C4)c3)ncnc21)c1ccc(N2Cc3ccccc3C2=O)cc1 ; CCS(=O)(=O)N1CC(CC#N)(n2cc(-c3ncnc4c3ccn4COC(=O)c3ccccc3Nc3cccc(C)c3C)cn2)C1 ; CCS(=O)(=O)N1CC(CC#N)(n2cc(-c3ncnc4c3ccn4COC(=O)c3ccccc3Nc3cccc(Cl)c3C)cn2)C1]

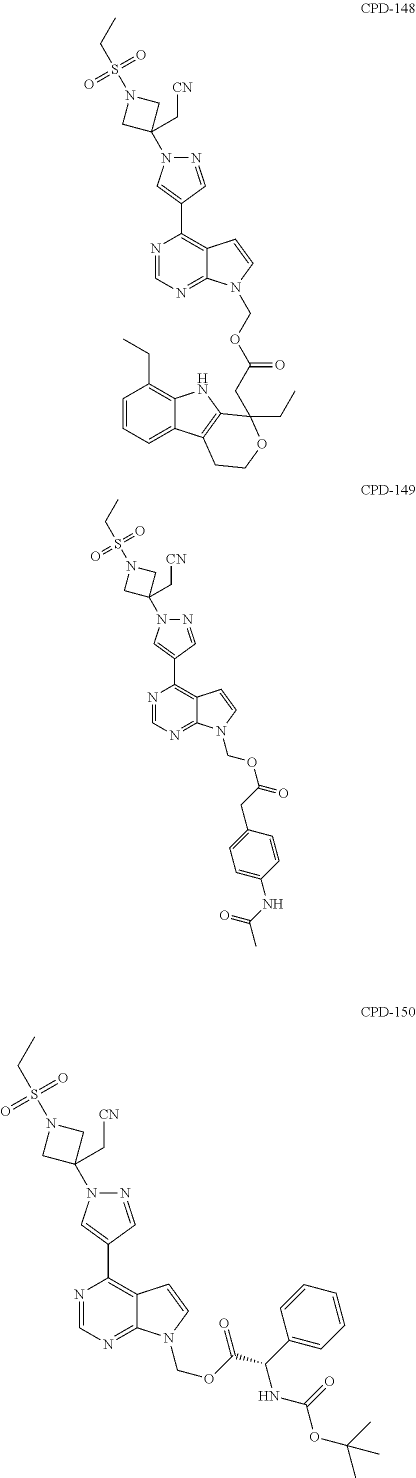 [SMILES: CCc1cccc2c3c([nH]c12)C(CC)(CC(=O)OCn1ccc2c(-c4cnn(C5(CC#N)CN(S(=O)(=O)CC)C5)c4)ncnc21)OCC3 ; CCS(=O)(=O)N1CC(CC#N)(n2cc(-c3ncnc4c3ccn4COC(=O)Cc3ccc(NC(C)=O)cc3)cn2)C1 ; CCS(=O)(=O)N1CC(CC#N)(n2cc(-c3ncnc4c3ccn4COC(=O)[C@@H](NC(=O)OC(C)(C)C)c3ccccc3)cn2)C1]

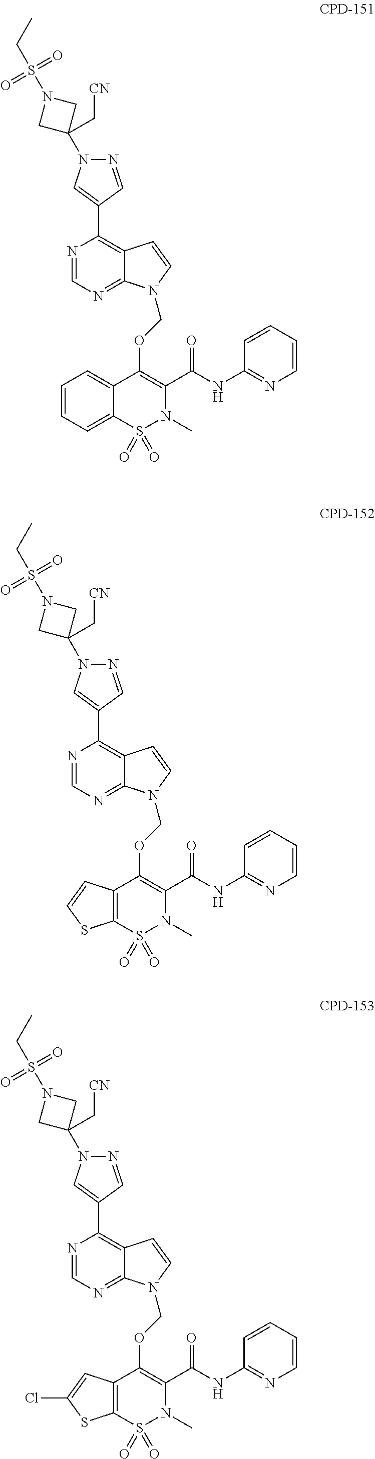 [SMILES: CCS(=O)(=O)N1CC(CC#N)(n2cc(-c3ncnc4c3ccn4COC3=C(C(=O)Nc4ccccn4)N(C)S(=O)(=O)c4ccccc43)cn2)C1 ; CCS(=O)(=O)N1CC(CC#N)(n2cc(-c3ncnc4c3ccn4COC3=C(C(=O)Nc4ccccn4)N(C)S(=O)(=O)c4sccc43)cn2)C1 ; CCS(=O)(=O)N1CC(CC#N)(n2cc(-c3ncnc4c3ccn4COC3=C(C(=O)Nc4ccccn4)N(C)S(=O)(=O)c4sc(Cl)cc43)cn2)C1]

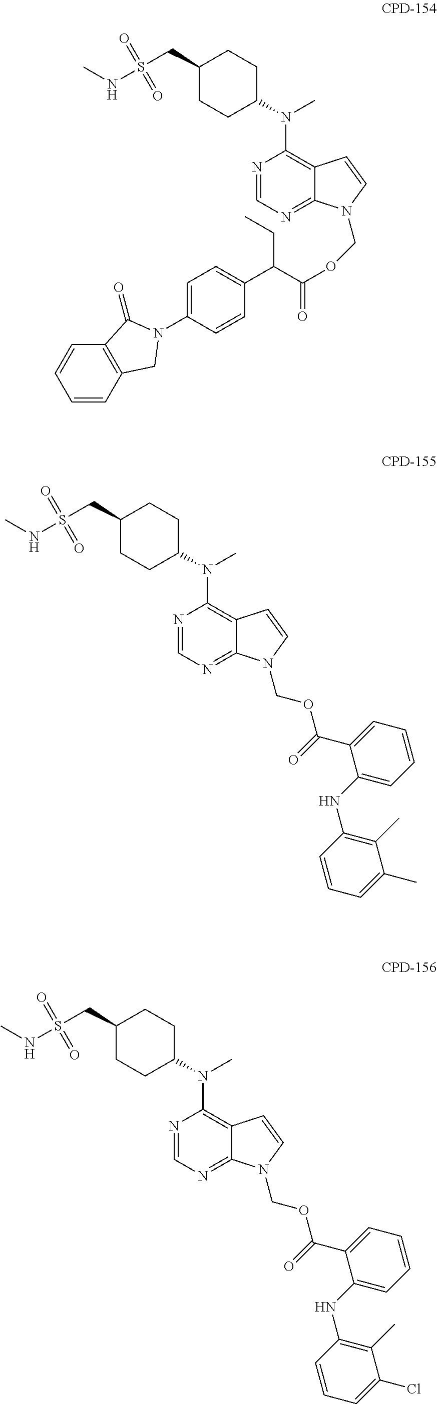

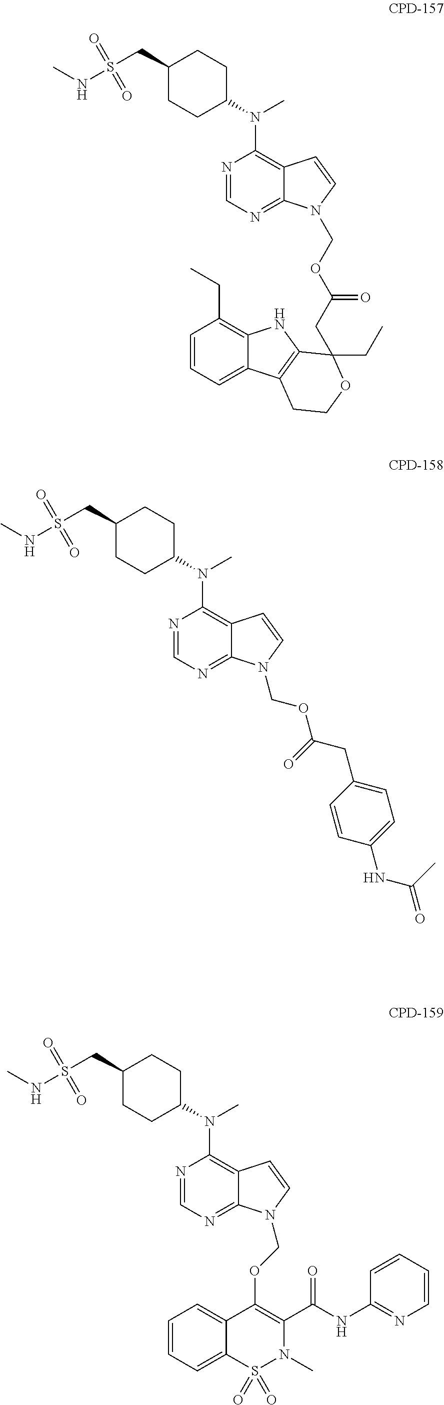

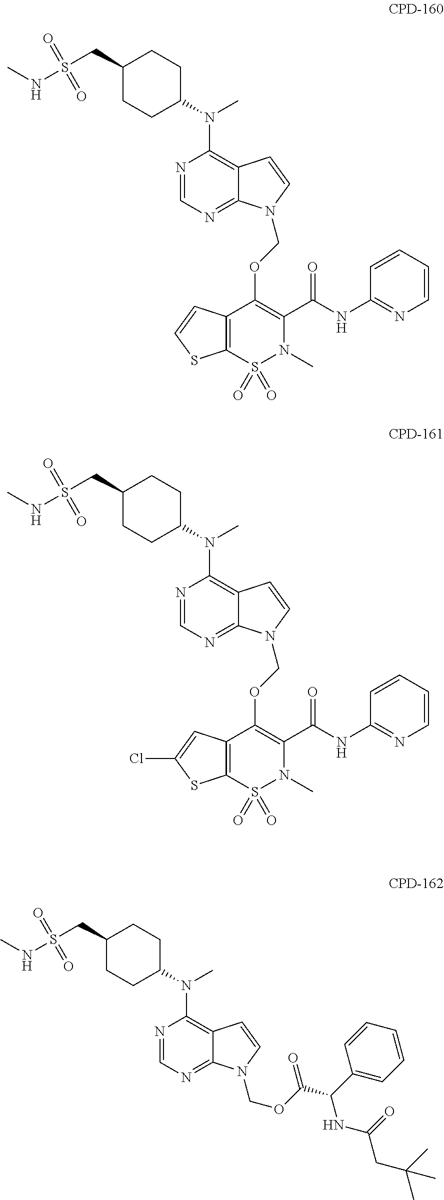

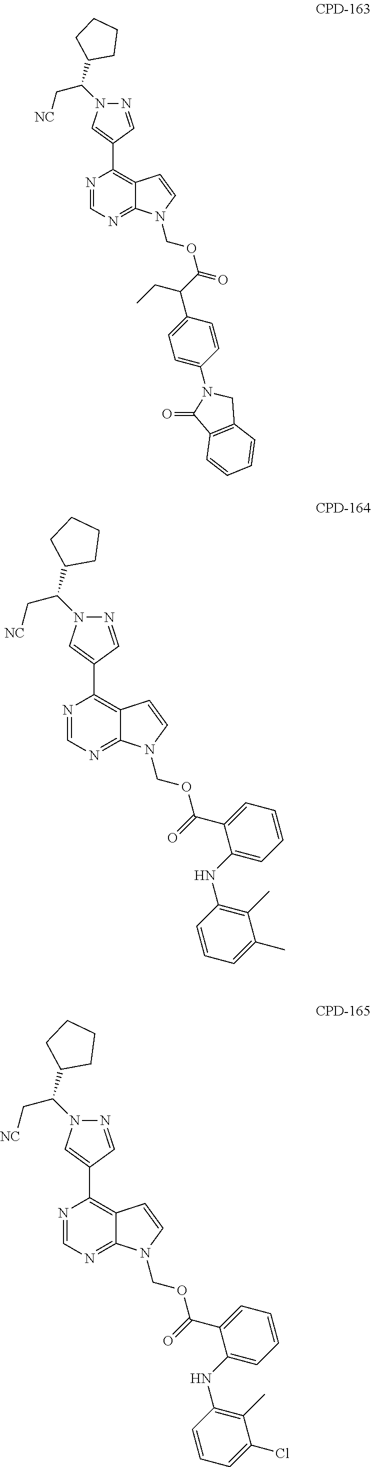 [SMILES: CCC(C(=O)OCn1ccc2c(-c3cnn([C@H](CC#N)C4CCCC4)c3)ncnc21)c1ccc(N2Cc3ccccc3C2=O)cc1 ; Cc1cccc(Nc2ccccc2C(=O)OCn2ccc3c(-c4cnn([C@H](CC#N)C5CCCC5)c4)ncnc32)c1C ; Cc1c(Cl)cccc1Nc1ccccc1C(=O)OCn1ccc2c(-c3cnn([C@H](CC#N)C4CCCC4)c3)ncnc21]

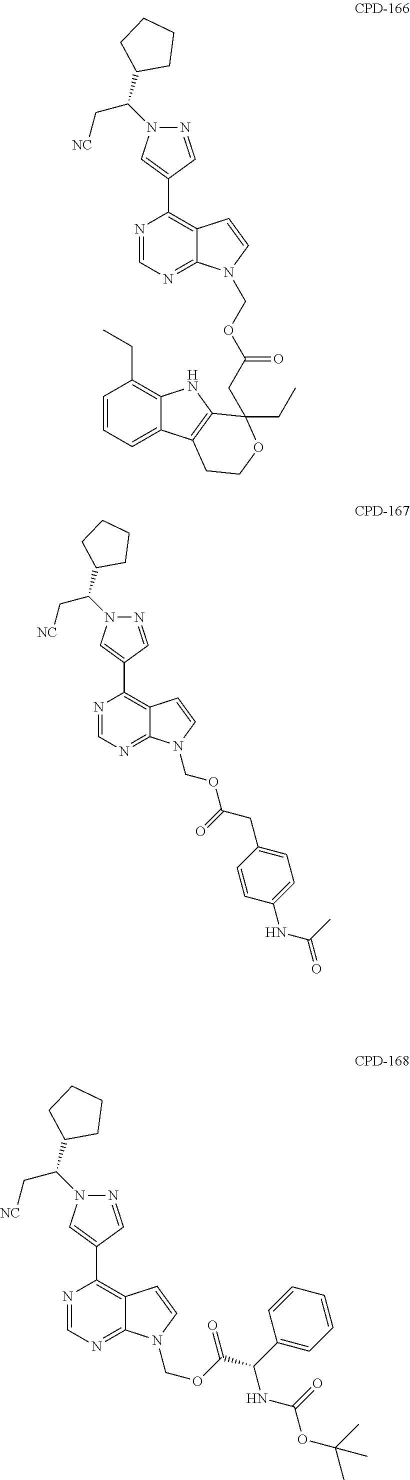 [SMILES: CCc1cccc2c3c([nH]c12)C(CC)(CC(=O)OCn1ccc2c(-c4cnn([C@H](CC#N)C5CCCC5)c4)ncnc21)OCC3 ; CC(=O)Nc1ccc(CC(=O)OCn2ccc3c(-c4cnn([C@H](CC#N)C5CCCC5)c4)ncnc32)cc1 ; CC(C)(C)OC(=O)N[C@H](C(=O)OCn1ccc2c(-c3cnn([C@H](CC#N)C4CCCC4)c3)ncnc21)c1ccccc1]

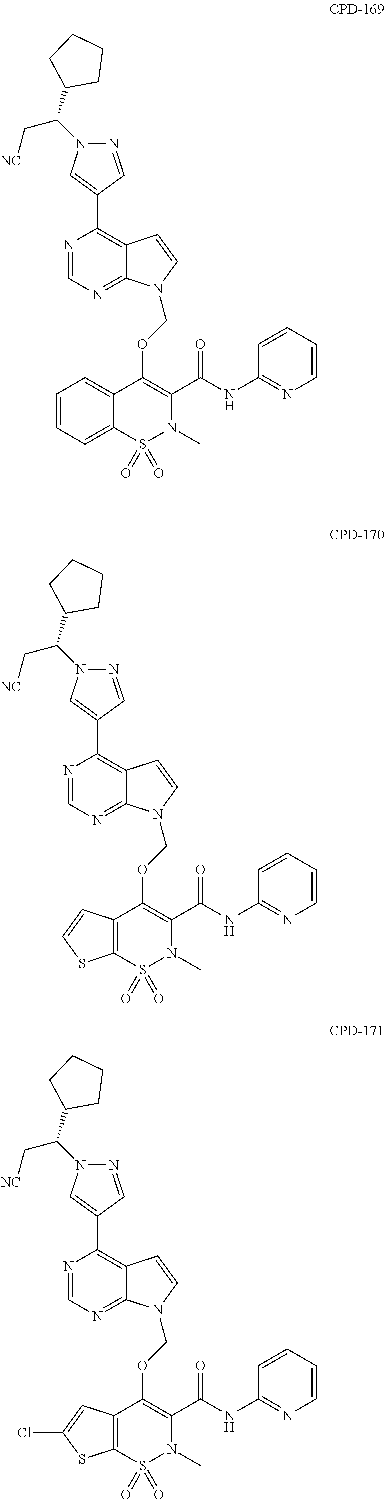 [SMILES: CN1C(C(=O)Nc2ccccn2)=C(OCn2ccc3c(-c4cnn([C@H](CC#N)C5CCCC5)c4)ncnc32)c2ccccc2S1(=O)=O ; CN1C(C(=O)Nc2ccccn2)=C(OCn2ccc3c(-c4cnn([C@H](CC#N)C5CCCC5)c4)ncnc32)c2ccsc2S1(=O)=O ; CN1C(C(=O)Nc2ccccn2)=C(OCn2ccc3c(-c4cnn([C@H](CC#N)C5CCCC5)c4)ncnc32)c2cc(Cl)sc2S1(=O)=O]

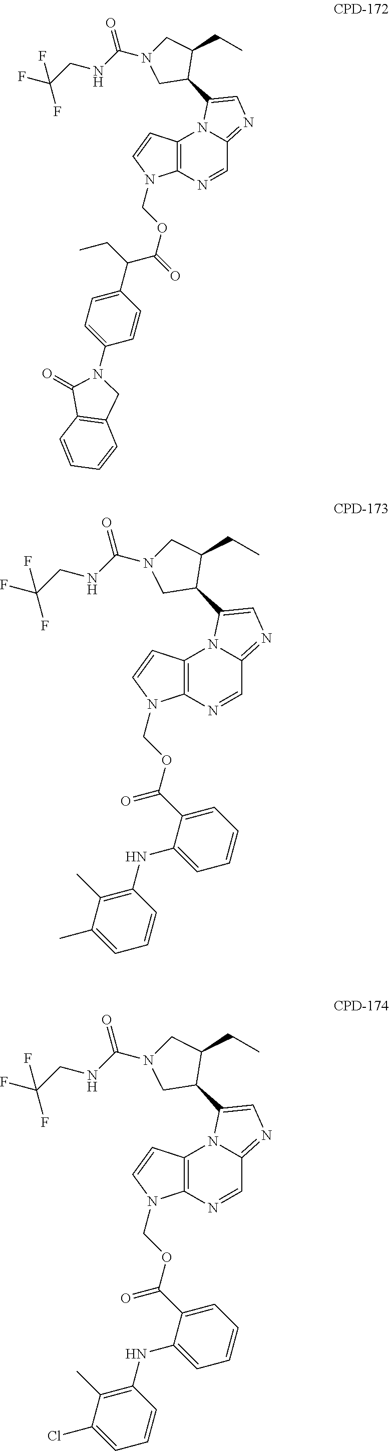 [SMILES: CCC(C(=O)OCn1ccc2c1ncc1ncc([C@H]3CN(C(=O)NCC(F)(F)F)C[C@H]3CC)n12)c1ccc(N2Cc3ccccc3C2=O)cc1 ; CC[C@@H]1CN(C(=O)NCC(F)(F)F)C[C@@H]1c1cnc2cnc3c(ccn3COC(=O)c3ccccc3Nc3cccc(C)c3C)n12 ; CC[C@@H]1CN(C(=O)NCC(F)(F)F)C[C@@H]1c1cnc2cnc3c(ccn3COC(=O)c3ccccc3Nc3cccc(Cl)c3C)n12]

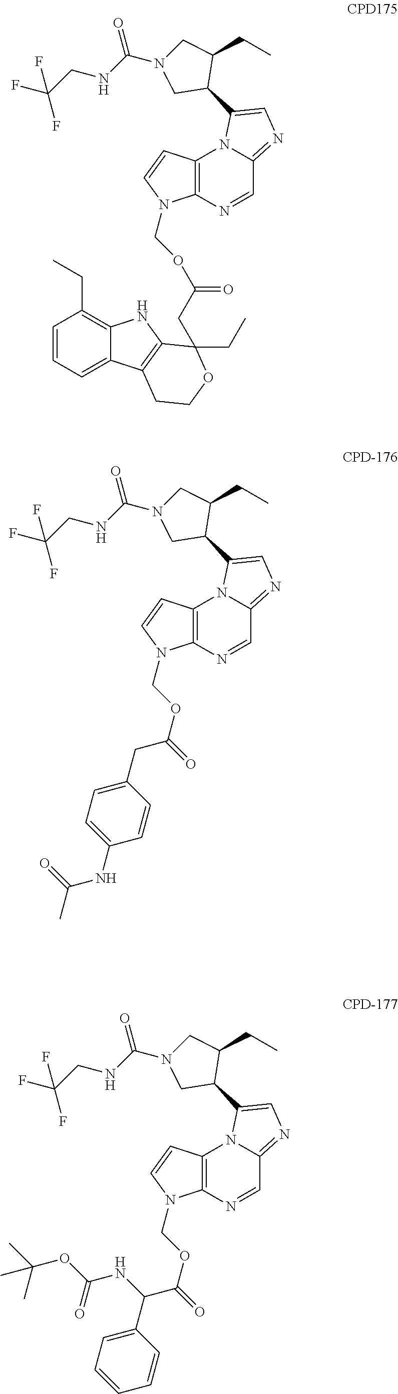 [SMILES: CCc1cccc2c3c([nH]c12)C(CC)(CC(=O)OCn1ccc2c1ncc1ncc([C@H]4CN(C(=O)NCC(F)(F)F)C[C@H]4CC)n12)OCC3 ; CC[C@@H]1CN(C(=O)NCC(F)(F)F)C[C@@H]1c1cnc2cnc3c(ccn3COC(=O)Cc3ccc(NC(C)=O)cc3)n12 ; CC[C@@H]1CN(C(=O)NCC(F)(F)F)C[C@@H]1c1cnc2cnc3c(ccn3COC(=O)C(NC(=O)OC(C)(C)C)c3ccccc3)n12]

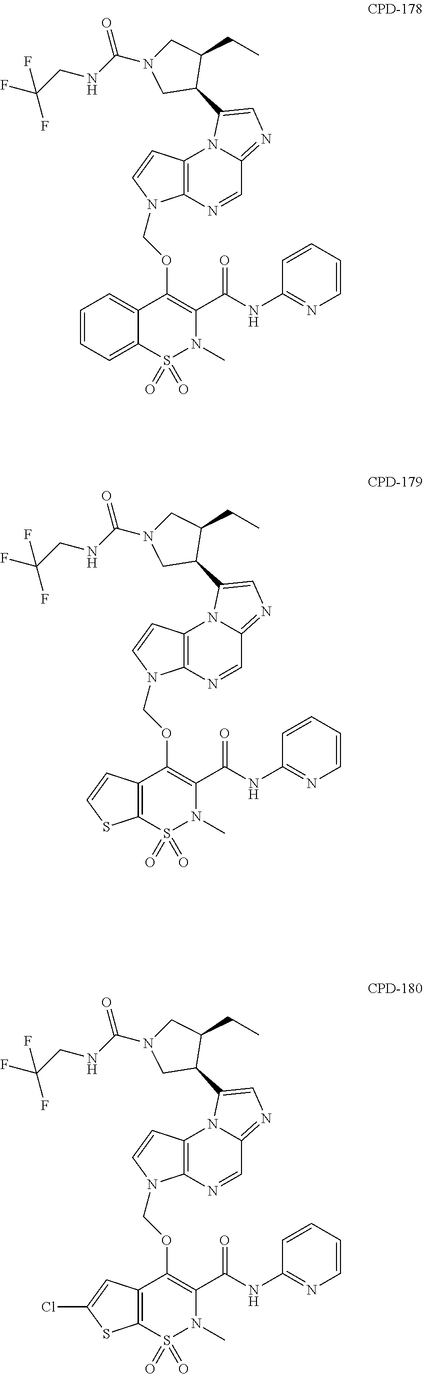 [SMILES: CC[C@@H]1CN(C(=O)NCC(F)(F)F)C[C@@H]1c1cnc2cnc3c(ccn3COC3=C(C(=O)Nc4ccccn4)N(C)S(=O)(=O)c4ccccc43)n12 ; CC[C@@H]1CN(C(=O)NCC(F)(F)F)C[C@@H]1c1cnc2cnc3c(ccn3COC3=C(C(=O)Nc4ccccn4)N(C)S(=O)(=O)c4sccc43)n12 ; CC[C@@H]1CN(C(=O)NCC(F)(F)F)C[C@@H]1c1cnc2cnc3c(ccn3COC3=C(C(=O)Nc4ccccn4)N(C)S(=O)(=O)c4sc(Cl)cc43)n12]

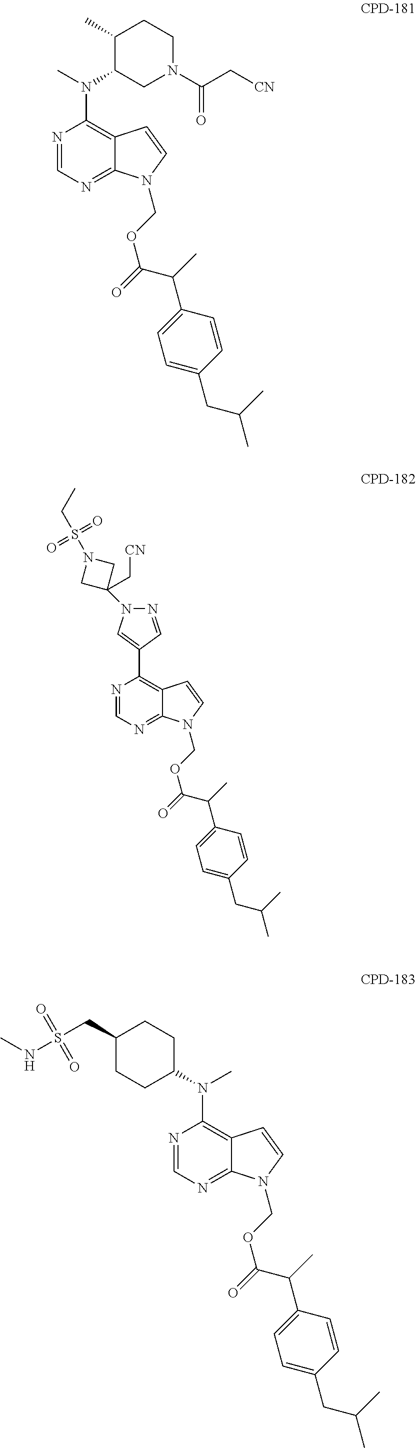

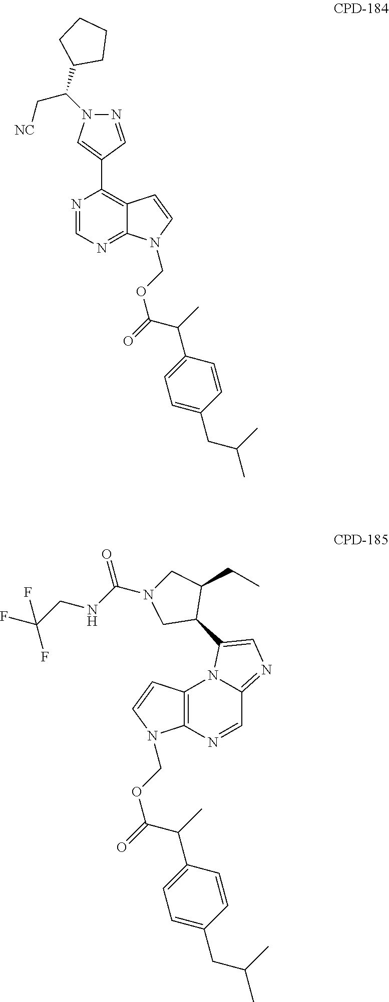 [SMILES: CC(C)Cc1ccc(C(C)C(=O)OCn2ccc3c(-c4cnn([C@H](CC#N)C5CCCC5)c4)ncnc32)cc1 ; CC[C@@H]1CN(C(=O)NCC(F)(F)F)C[C@@H]1c1cnc2cnc3c(ccn3COC(=O)C(C)c3ccc(CC(C)C)cc3)n12]

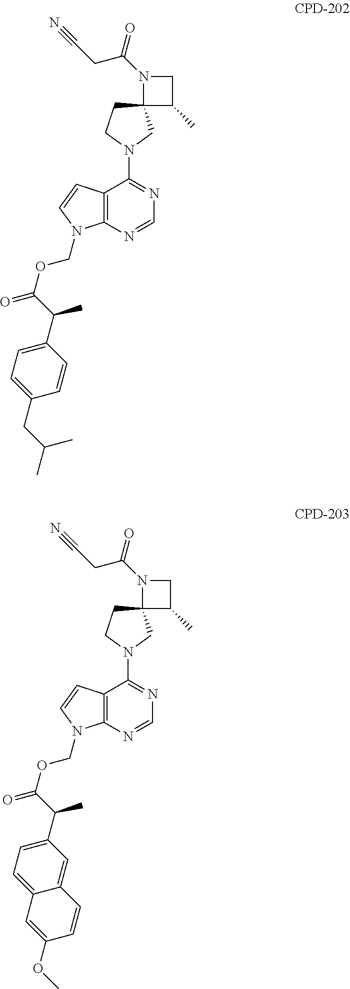 [SMILES: CC(C)Cc1ccc([C@H](C)C(=O)OCn2ccc3c(N4CC[C@]5(C4)[C@@H](C)CN5C(=O)CC#N)ncnc32)cc1 ; COc1ccc2cc([C@H](C)C(=O)OCn3ccc4c(N5CC[C@]6(C5)[C@@H](C)CN6C(=O)CC#N)ncnc43)ccc2c1]

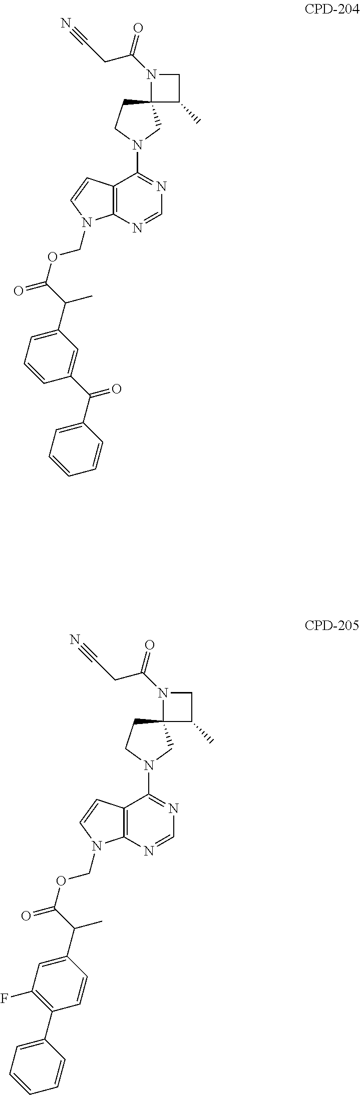 [SMILES: CC(C(=O)OCn1ccc2c(N3CC[C@]4(C3)[C@@H](C)CN4C(=O)CC#N)ncnc21)c1cccc(C(=O)c2ccccc2)c1 ; CC(C(=O)OCn1ccc2c(N3CC[C@]4(C3)[C@@H](C)CN4C(=O)CC#N)ncnc21)c1ccc(-c2ccccc2)c(F)c1]

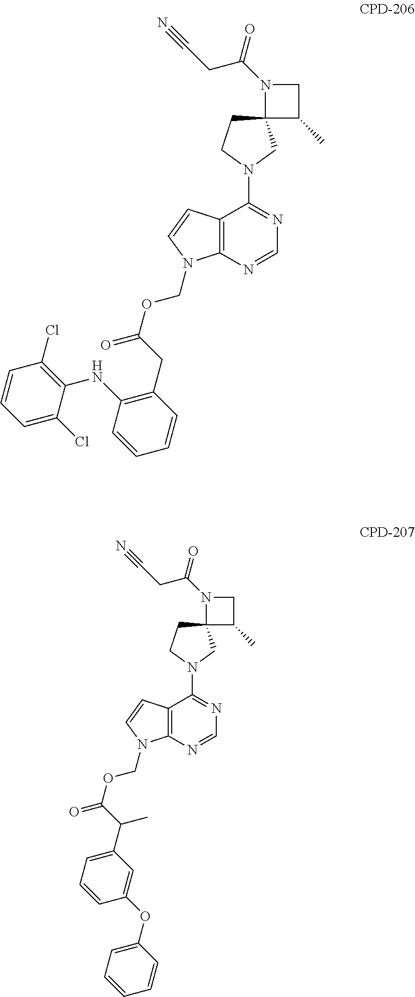 [SMILES: C[C@H]1CN(C(=O)CC#N)[C@]12CCN(c1ncnc3c1ccn3COC(=O)Cc1ccccc1Nc1c(Cl)cccc1Cl)C2 ; CC(C(=O)OCn1ccc2c(N3CC[C@]4(C3)[C@@H](C)CN4C(=O)CC#N)ncnc21)c1cccc(Oc2ccccc2)c1]

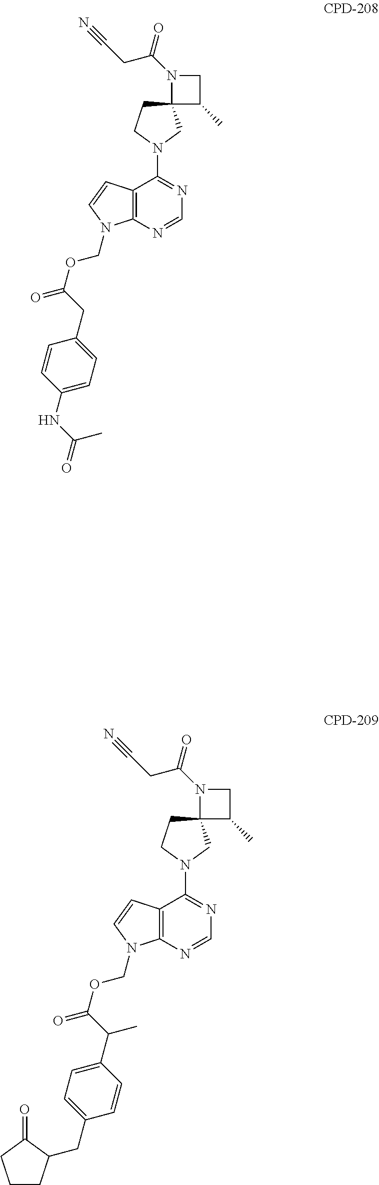 [SMILES: CC(=O)Nc1ccc(CC(=O)OCn2ccc3c(N4CC[C@]5(C4)[C@@H](C)CN5C(=O)CC#N)ncnc32)cc1 ; CC(C(=O)OCn1ccc2c(N3CC[C@]4(C3)[C@@H](C)CN4C(=O)CC#N)ncnc21)c1ccc(CC2CCCC2=O)cc1]

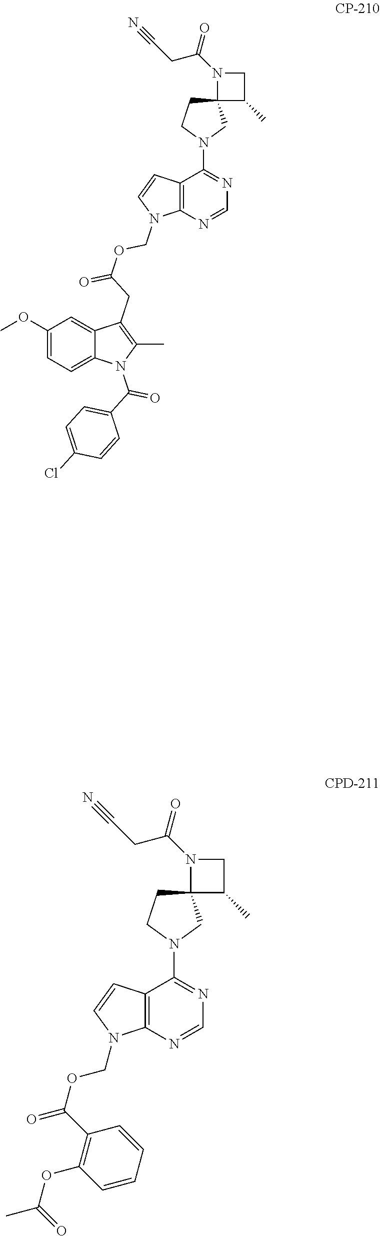

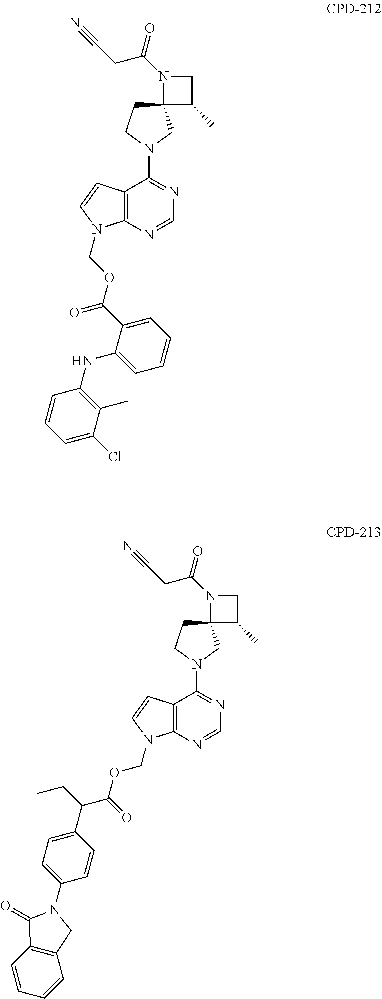 [SMILES: Cc1c(Cl)cccc1Nc1ccccc1C(=O)OCn1ccc2c(N3CC[C@]4(C3)[C@@H](C)CN4C(=O)CC#N)ncnc21 ; CCC(C(=O)OCn1ccc2c(N3CC[C@]4(C3)[C@@H](C)CN4C(=O)CC#N)ncnc21)c1ccc(N2Cc3ccccc3C2=O)cc1]

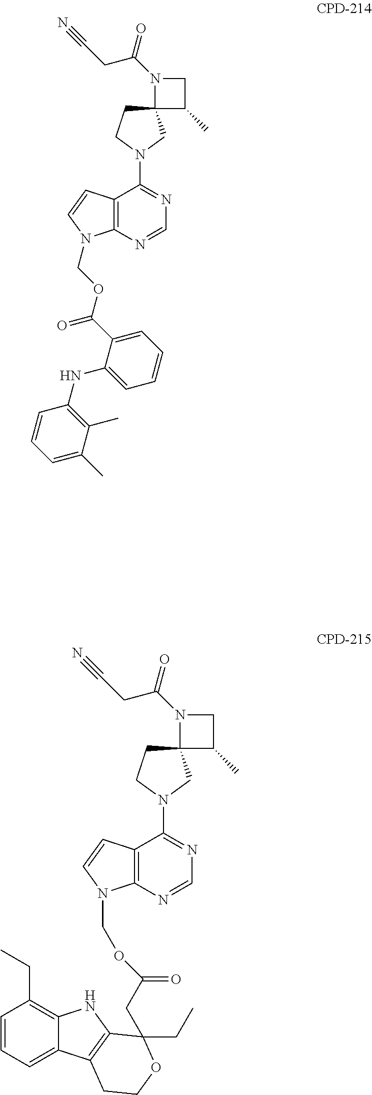 [SMILES: Cc1cccc(Nc2ccccc2C(=O)OCn2ccc3c(N4CC[C@]5(C4)[C@@H](C)CN5C(=O)CC#N)ncnc32)c1C ; CCc1cccc2c3c([nH]c12)C(CC)(CC(=O)OCn1ccc2c(N4CC[C@]5(C4)[C@@H](C)CN5C(=O)CC#N)ncnc21)OCC3]

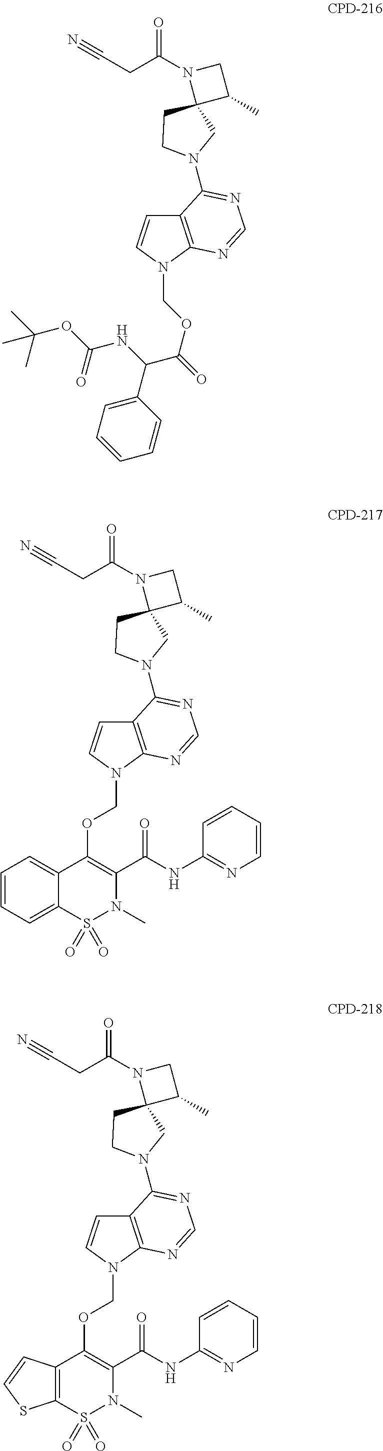 [SMILES: C[C@H]1CN(C(=O)CC#N)[C@]12CCN(c1ncnc3c1ccn3COC(=O)C(NC(=O)OC(C)(C)C)c1ccccc1)C2 ; C[C@H]1CN(C(=O)CC#N)[C@]12CCN(c1ncnc3c1ccn3COC1=C(C(=O)Nc3ccccn3)N(C)S(=O)(=O)c3ccccc31)C2 ; C[C@H]1CN(C(=O)CC#N)[C@]12CCN(c1ncnc3c1ccn3COC1=C(C(=O)Nc3ccccn3)N(C)S(=O)(=O)c3sccc31)C2]

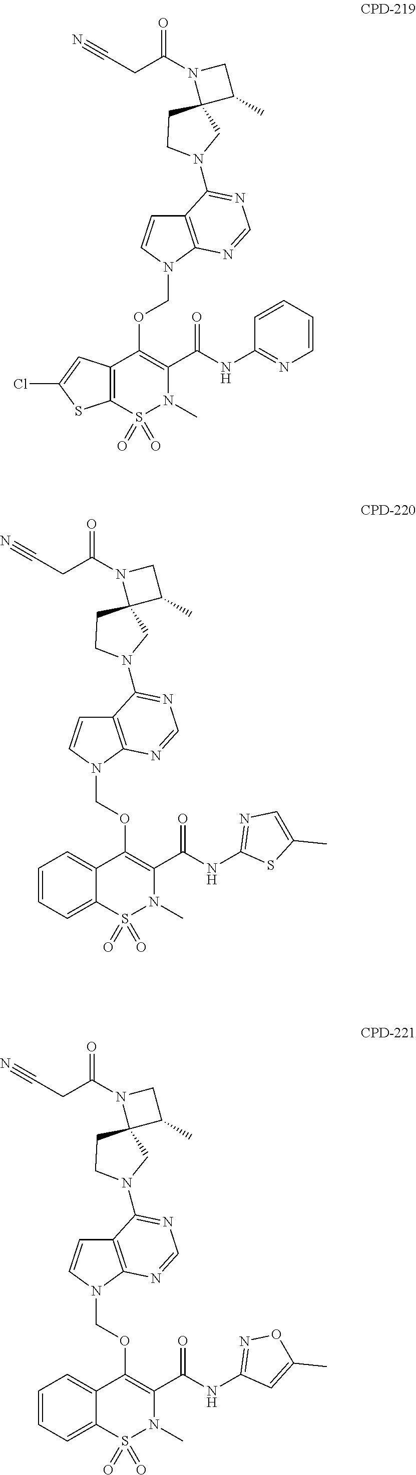 [SMILES: C[C@H]1CN(C(=O)CC#N)[C@]12CCN(c1ncnc3c1ccn3COC1=C(C(=O)Nc3ccccn3)N(C)S(=O)(=O)c3sc(Cl)cc31)C2 ; Cc1cnc(NC(=O)C2=C(OCn3ccc4c(N5CC[C@]6(C5)[C@@H](C)CN6C(=O)CC#N)ncnc43)c3ccccc3S(=O)(=O)N2C)s1 ; Cc1cc(NC(=O)C2=C(OCn3ccc4c(N5CC[C@]6(C5)[C@@H](C)CN6C(=O)CC#N)ncnc43)c3ccccc3S(=O)(=O)N2C)no1]

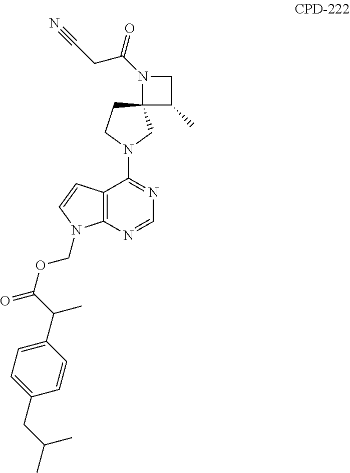 [SMILES: CC(C)Cc1ccc(C(C)C(=O)OCn2ccc3c(N4CC[C@]5(C4)[C@@H](C)CN5C(=O)CC#N)ncnc32)cc1]

On the other hand, the present invention also provides a method for preparing the anti-inflammatory compound, or the stereoisomer, tautomer, nitrogen oxide, metabolite, prodrug, pharmaceutically acceptable salt, or solvate thereof of the present invention, comprising the steps of:
subjecting A and B to a condensation reaction accompanied by the loss of water in the presence of a catalyst and an organic solvent.

Preferably, for the preparation method of the present invention, the catalyst is one or two or more of EDCI (1-(3-dimethylaminopropyl)-3-ethylcarbodiimide hydrochloride)), DCC (dicyclohexyl carbodiimide), CDI (N, N-carbonyldiimidazole), DMTMM (4-(4, 6-dimethoxytriazine)-4-methylmorpholine hydrochloride), HATU (2-(7-azabenzotriazole)-N, N, N′, N′-tetramethyluronium hexafluorophosphate), HCTU (6-chlorobenzotriazole-1, 1, 3, 3-tetramethyluronium hexafluorophosphate), PyBOP (benzotriazol-1-yl-oxytripyrrolidinophosphonium hexafluorophosphate) and NPC; preferably the organic solvent is one or two or more selected from a group consisting of DCM (dichloromethane), DMF (dimethylformamide), petroleum ether, acetone, chloroform, ethyl acetate, acetonitrile and THF (tetrahydrofuran), more preferably dichloromethane and/or dimethylformamide; further preferred, the reaction is carried out in the presence of a basic substance, wherein the basic substance is preferably one or two or more selected from a group consisting of DMAP (dimethylamino pyridine), triethylamine, DIPEA (N, N-diisopropylethyl amine) and hydroxides or salts of sodium, potassium, lithium and ammonium.

Preferably, the method for preparing the anti-inflammatory compound, or the stereoisomer, tautomer, nitrogen oxide, metabolite, prodrug, pharmaceutically acceptable salt, or solvate thereof of the present invention, comprises the steps of subjecting A-CH₂OH and B to a condensation reaction on the loss of water.

Preferably, the method for preparing the anti-inflammatory compound, or the stereoisomer, tautomer, nitrogen oxide, metabolite, prodrug, pharmaceutically acceptable salt, or solvate thereof of the present invention, obtained by a preparation method comprising the steps of:
2) reacting A-CH₂—OH compound with an acyl chloride of B or directly with the B compound;
wherein the A-CH₂—OH compound is preferably prepared by the following step 1):
reacting the amine compound A to generate an A-CH₂—OH compound.

More preferably, for the preparation process according to the invention, wherein step 1) comprises: a) adding (2-(chloromethoxy) ethyl) trimethylsilane to A in the presence of a catalyst and a solvent to generate A-CH₂O—C₂H₄—Si(CH₃)₃; and b) generating A-CH₂OH from A-CH₂O—C₂H₄—Si(CH₃)₃ in the presence of a catalyst and a solvent; step 2) comprises: reacting A-CH₂OH with an acid chloride generated from compound B₁ or directly with compound B₂ to generate A-CH₂O—B;
wherein it is preferred that in step b) the reaction is carried out in the presence of TFA (trifluoroacetic acid) as a catalyst and DCM (dichloromethane) as a solvent; or in step 2), A-CH₂OH is reacted with the acid chloride generated from the compound B₁ in the presence of Et₃N (triethylamine) as a catalyst and DCM (dichloromethane) as a solvent or is reacted with compound B₂ in the presence of PPh3 (triphenylphosphine) and DIAD (diisopropyl azodicarboxylate) as a catalyst and THF (tetrahydrofuran) as a solvent.

Furthermore, the present invention provides a medicinal use, i.e. provides use of the anti-inflammatory compound, or the stereoisomer, tautomer, nitrogen oxide, metabolite, prodrug, pharmaceutically acceptable salt, or solvate thereof, in the preparation of anti-inflammatory drug preparations or drug compositions (preferably external drug compositions).

Furthermore, the present invention also provides an anti-inflammatory drug preparation or drug composition (preferably external drug compositions) including the anti-inflammatory compound, or the stereoisomer, tautomer, nitrogen oxide, metabolite, prodrug, pharmaceutically acceptable salt, or solvate thereof described above.

It has been found that, by synthesizing the above compounds, the compounds of the present invention have a dissolution rate of 0.04% to 50% per day, thereby increasing the transdermal potential of known JAK inhibitors.

### BRIEF DESCRIPTION OF THE DRAWINGS

FIG. 1 is a graph of the PASI score of an ointment prepared from CPD-029 of the present invention through skin application to treat psoriasis in a mouse model test;

FIG. 2 is a graph of the PASI score of an ointment prepared from CPD-028 of the present invention through skin application to treat psoriasis in a mouse model test;

FIG. 3 is a graph of the PASI score of an ointment prepared from CPD-027 of the present invention through skin application to treat psoriasis in a mouse model test;

FIG. 4 is a graph of the PASI score of an ointment prepared from CPD-017 of the present invention through skin application to treat psoriasis in a mouse model test; and

FIG. 5 is a graph of the PASI score of an ointment prepared from CPD-002 of the present invention through skin application to treat psoriasis in a mouse model test.

### DETAILED DESCRIPTION OF THE INVENTION

The present inventors have unexpectedly discovered through intensive studies that the coupling of an anti-inflammatory pharmaceutical compound containing a carboxylic acid or hydroxyl group with a JAK inhibitor compound to form a coupling compound having an acyloxy group and/or methoxy group has a high therapeutic effect and a particular effect of controlled-release pharmaceutical activity.

The anti-inflammatory compound, or a stereoisomer, tautomer, nitrogen oxide, metabolite, prodrug, pharmaceutically acceptable salt, or solvate thereof provided by the present invention, has a structure shown in general formula (I):

A-Y—B (I)
wherein, A is a group after dehydrogenation of an amine compound having JAK inhibitory activity;
Y is a direct connection or —(CH₂)—O—;
B is a group formed by means of dehydroxylation of a carboxylic acid compound B₁ with anti-inflammatory effect, or a group formed by means of dehydrogenation of a hydroxy-containing compound B₂.

That is to say, the compounds of general formula (I) provided by the present invention actually include two main classes. The first main class refers to the case where A is a direct connection and the structural formula thereof is as shown in (II) or (IIa).

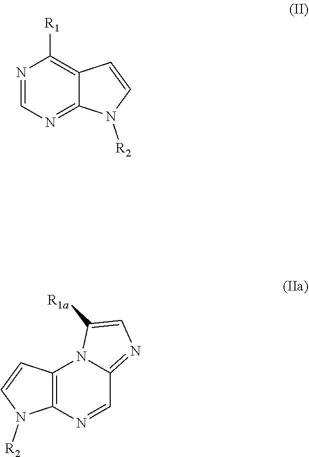

wherein R₁ is selected from pyrazolyl or —N(CH₃)—Cy unsubstituted or substituted with Rₐ; R₁ₐ represents a pyrrole ring substituted a halogen-substituted C₁-C₆ alkylaminoacyl group and/or a C₁-C₆ alkyl group;
C_{y} is a five- or six-membered carbocyclic ring or a five- or six-membered nitrogen-containing heterocyclic ring unsubstituted or substituted by R_{b}; Rₐ and R_{b} are each independently at least one or two groups containing an acyl group, a sulfonyl group, a cyano group, an amino group or a C₁-C₆ alkyl-substituted amino group; preferably Rₐ and R_{b} are each independently one of acyl or sulfonyl and at least one group selected from cyano, amino, or C₁-C₆ alkyl substituted amino, wherein the C₁-C₆ alkyl is substitutable by halogen;
R₂ in both general formulas (II) and (IIa) is —B, i.e. is a group formed by means of dehydroxylation of a carboxy-containing carboxylic acid compound B₁ and is selected from R₄—Ar—R₃—CO—, wherein R₃ is selected from C₁-C₄ alkylene; —NH—, R₅NH—, or C₁-C₆ alkylene substituted with a C₁-C₆ alkoxyamide group; or a direct connection, i.e. the Ar group is directly linked to —CO—; R₃ is preferably methyl substituted or unsubstituted methylene, —C₂H₄—, or a direct connection; R₅ is C₁-C₆ alkylene; wherein the C₁-C₆ alkylene is substitutable by halogen (preferably halogen is selected from one or two or more of fluorine, chlorine or bromine);
Ar is an aromatic ring group, preferably selected from a benzene ring; a naphthalene ring or an aryl heterocyclic ring; and a benzene ring, a naphthalene ring, or an aryl heterocyclic ring or an aryl fused heterocyclic ring (here, the aryl heterocyclic ring being preferably a benzo nitrogen- or oxygen-containing heterocyclic ring such as a benzopyrrole ring) substituted with a group selected from halogen, a C₁-C₆ alkyl group, a C₁-C₆ alkoxy group, a C₁-C₆ acyl group, or a C₁-C₆ alkoxy group; Ar is more preferably an aryl heterocyclic ring containing a nitrogen atom; and
R₆ is halogen, C₁-C₆ alkyl, C₁-C₆ alkoxy, or C₁-C₆ cycloalkanoyl-containing C₁-C₆ alkyl;
or R₆ is C₁-C₆ alkyl or aromatic ring or aromatic condensed ring or aromatic condensed ring heterocyclic ring containing acyl and/or amino groups, such as C₁-C₆ alkylamido or aryl fused heterocyclic amido, C₁-C₆ carbonyloxy, halogen-substituted benzoyl, C₁-C₆ alkyl or halogen substituted or unsubstituted phenoxy, C₁-C₆ alkyl or halogen substituted or unsubstituted phenyl or aryl fused heterocyclic ring, C₁-C₆ alkyl or halogen substituted or unsubstituted phenylamino, or R₆ can also be absent; wherein the C₁-C₆ alkoxy can also form a bridged ring with Ar.

The aforementioned terms in the present invention, as well as all “C₁-C₆” appearing below, refer to a carbon atom number of 1-6. For example, “C₁-C₆ alkyl” refers to an alkyl group with a carbon atom number of 1-6. By analogy, the terms “C₁-C₆ alkoxy” and “C₁-C₆ acyl” mentioned herein refer to alkoxy groups with a carbon atom number of 1-6 and groups with a carbon atom number of 1-6 containing —C═O, respectively. “C₁-C₆ alkylamide group” refers to an alkyl group containing amide groups with a carbon atom number of 1-6. “C₁-C₆ carboacyloxy group” refers to an alkyl group or cycloalkyl group containing acyloxy —CO—O with a carbon atom number of 1-6. “C₁-C₆ cycloalkanoyl group” refers to a cyclic alkyl group containing —C═O group with a carbon atom number of 1-6. The term “C₁-C₆ alkyl group containing C₁-C₆ cycloalkanoyl group” refers to the situation where hydrogen on carbon atoms is substituted by C₁-C₆ cycloalkanoyl groups in an alkyl group with a carbon atom number of 1-6, i.e., “C₁-C₆ alkyl group containing C₁-C₆ cycloalkanoyl group” is equivalent to “C₁-C₆ alkyl group substituted by C₁-C₆ cycloalkanoyl group”. The “C₁-C₆” in the present invention may be specifically “C₁-C₆”, “C₁-C₅”, “C₁-C₄”, “C₁-C₃” or “C₁-C₂”, and may also be C₁, i.e. only one carbon atom.

The first general compound described above is a compound A-B (specifically, A-B₁) formed by means of dehydrogenation of an amine compound A having JAK inhibitory activity and dehydroxylation of a carboxylic acid compound B₁.

The compound of general formula (I) provided by the present invention includes the second main class of compound having a structural formula and structure shown in general formula (III):

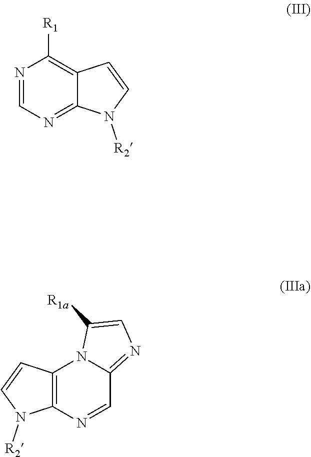

wherein R₁ has the same meaning as R₁ in general formula (II); R₁ₐ has the same meaning as R₁ₐ in formula (IIa);
The R₂′ in general formula (III) and general formula (IIIa) are both —Y—B, where Y is —(CH₂)—O—, and B is —B₁ in general formula (II) or (IIa), i.e. group —B1 formed by the dehydroxylation of a carboxylic acid compound B₁; or B is —B₂, the group —B₂ is R—CO—NH—R_{d}, wherein R_{c} is a 4-hydroxy-benzothiazine dioxide-3-yl represented by the following structural formula (a), wherein the phenyl ring is substitutable by halogen or C₁-C₄ alkyl, or a 4-hydroxy-Re substituted thienothiazine dioxide-3-yl represented by the following structural formula (b), wherein —CO—NH—R_{d} is attached at the 3-position of the thiazine ring,

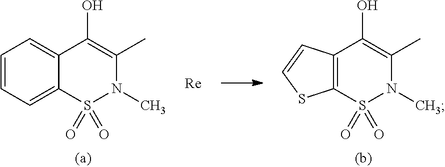

wherein R_{d} is thiazole, isothiazole, oxazole, isoxazole, or pyridine or a group thereof substituted with C₁-C₆ alkyl or halogen, preferably a thiazole or isoxazole substituted with methyl; and unsubstituted pyridyl; Rₑ is C₁-C₆ alkyl or halogen (preferably halogen is selected from one or two or more of fluorine, chlorine, or bromine); the arrow next to Rₑ in formula (b) indicates that its substitution position on the thiophene ring may be any carbon-linked hydrogen atom capable of undergoing substitution.

The second main compound of the present invention is obtained by using A-CH₂OH to undergo a condensation reaction accompanied by loss of water with B.

In particular, the preparation of the second main class of compound comprises the following steps:
2) reacting A-CH₂—OH compound with an acyl chloride of B or directly with the B compound; Wherein the A-CH₂—OH compound is prepared by step 1): reacting A to generate an A-CH₂—OH compound.

The scope of the compound of general formula (I) of the present invention actually also includes the various stereoisomers, tautomers, N-oxides, metabolites, prodrugs, pharmaceutically acceptable salts or solvates thereof, which can be obtained by those skilled in the art according to the common knowledge, that is to say, these compounds, and the various stereoisomers, tautomers, N-oxides, metabolites, prodrugs, pharmaceutically acceptable salts or solvates thereof and the like, which can be modified by those skilled in the art according to common knowledge, can be used in the present invention to achieve the particular effects of the compounds of the present invention, such as strong transdermal performance, controllable drug release, and high efficacy, and therefore all fall within the scope of the present invention.

Examples The following is an example to illustrate how the compound of the present invention is prepared and its performance evaluation.

### The NMR Instrument and Mass Spectrometer Instrument Models Used in Examples 1-149 Below are Described Below, Respectively:

Nuclear magnetic resonance spectrometer: Bruker 400M Nuclear Magnetic Resonance Instrument; Liquid Chromatography Mass Spectrometry Instrument: Agilent InfinityLab LC/MSD iQ Table 1 below is a structural formula and a compound name of a target compound prepared in each example.

Table 1 Structural formulas and compound names of target compounds prepared in examples

TABLE 1
Structural formulas and compound names of target compounds prepared in examples

| Examples | | |
|---|---|---|
| (Cmpd No.) | Structure | Chinese name |
| 1 (CPD-001) | 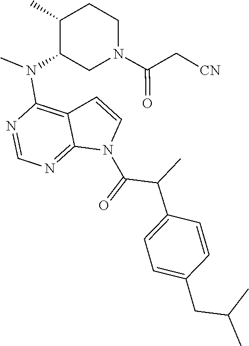 [SMILES: CC(C)Cc1ccc(C(C)C(=O)n2ccc3c(N(C)[C@H]4CN(C(=O)CC#N)CC[C@H]4C)ncnc32)cc1] | 3-((3R,4R)-3-((7-(2-(4-isobutylphenyl) propanoyl)-7H-pyrrolo [2,3-d]pyrimidin-4-yl) (methyl) amino)-4-methylpiperidin-1-yl)-3- oxopropanenitrile |
| 2 (CPD-002) | 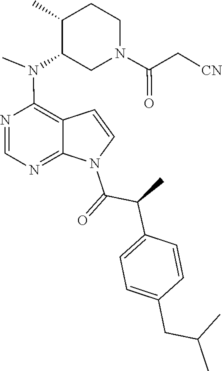 [SMILES: CC(C)Cc1ccc([C@H](C)C(=O)n2ccc3c(N(C)[C@H]4CN(C(=O)CC#N)CC[C@H]4C)ncnc32)cc1] | 3-((3R,4R)-3-((7-((S)-2-(4-isobutylphenyl) propanoyl)-7H-pyrrolo[2,3-d]pyrimidin-4-yl) (methyl) amino)-4-methylpiperidin-1-yl)-3- oxopropanenitrile |
| 3 (CPD-003) | 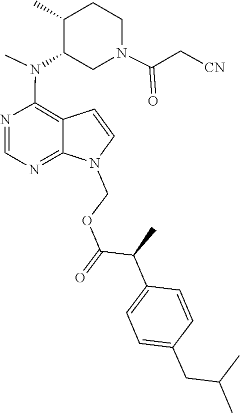 [SMILES: CC(C)Cc1ccc([C@H](C)C(=O)OCn2ccc3c(N(C)[C@H]4CN(C(=O)CC#N)CC[C@H]4C)ncnc32)cc1] | (4-((3R, 4R)-1-(2-cyanoacetyl)- 4-methylpiperidin-3-yl) (methyl)amino)-7H-pyrrolo[2,3-d] pyrimidin-7-yl)methyl (S)-2-(4-isobutylphenyl)propanoate |
| 4 (CPD-004) | 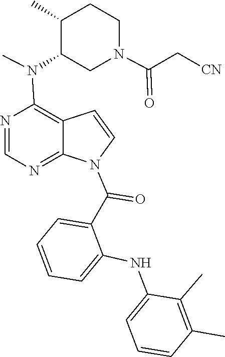 | 3-((3R,4R)-3-((7-(2-((2,3-dimethylphenyl) amino)benzoyl)-7H-pyrrolo[2,3-d]pyrimidin- 4-yl)(methyl) amino)-4-methylpiperidin-1-yl)-3- oxopropanenitrile |
| 5 (CPD-005) | 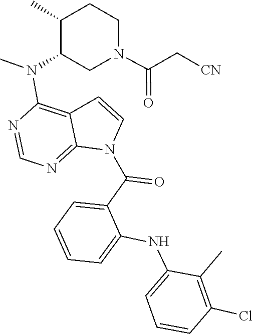 [SMILES: Cc1c(Cl)cccc1Nc1ccccc1C(=O)n1ccc2c(N(C)[C@H]3CN(C(=O)CC#N)CC[C@H]3C)ncnc21] | 3-((3R, 4R)-3-((7-(2-((3-chloro-2-methylphenyl) amino)benzoyl)-7H-pyrrolo[2,3-d] pyrimidin-4-yl)(methyl) amino)-4-methylpiperidin-1-yl)-3- oxopropanenitrile |
| 6 (CPD-006) | 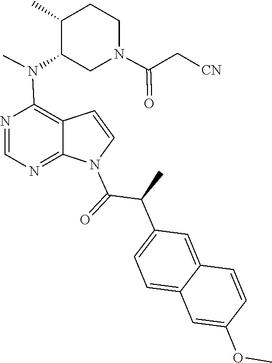 | 3-((3R, 4R)-3-((7-((S)-2-(6-methoxynaphthalen-2-yl) propanoyl)-7H-pyrrolo[2,3-d]pyrimidin-4-yl) (methyl) amino)-4-methylpiperidin-1-yl)-3- oxopropanenitrile |
| 7 (CPD-007) | 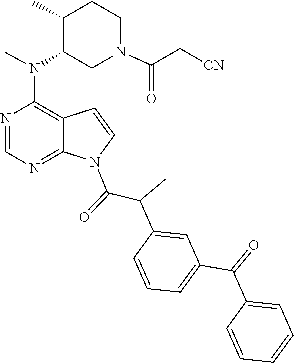 [SMILES: CC(C(=O)n1ccc2c(N(C)[C@H]3CN(C(=O)CC#N)CC[C@H]3C)ncnc21)c1cccc(C(=O)c2ccccc2)c1] | 3-((3R,4R)-3-((7-(2-(3-benzoylphenyl) propanoyl)-7H-pyrrolo[2,3-d]pyrimidin-4-yl) (methyl) amino)-4-methylpiperidin-1-yl)-3- oxopropanenitrile |
| 8 (CPD-008) | 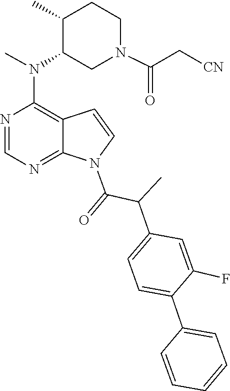 [SMILES: CC(C(=O)n1ccc2c(N(C)[C@H]3CN(C(=O)CC#N)CC[C@H]3C)ncnc21)c1ccc(-c2ccccc2)c(F)c1] | 3-((3R, 4R)-3-((7-(2-(2-fluoro-[1, 1′-biphenyl]-4-yl)propanoyl)-7H-pyrrolo[2, 3-d]pyrimidin-4-yl)(methyl) amino)-4-methylpiperidin-1-yl)-3- oxopropanenitrile |
| 9 (CPD-009) | 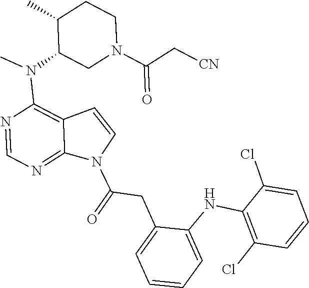 [SMILES: C[C@@H]1CCN(C(=O)CC#N)C[C@@H]1N(C)c1ncnc2c1ccn2C(=O)Cc1ccccc1Nc1c(Cl)cccc1Cl] | 3-((3R,4R)-3-((7-(2-(2-(2-(2-(2, 6-dichlorophenyl)amino)phenyl) acetyl)-7H-pyrrolo[2,3-d]pyrimidin-4-yl) (methyl) amino)-4-methylpiperidin-1-yl)-3- oxopropanenitrile |
| 10 (CPD-010) | 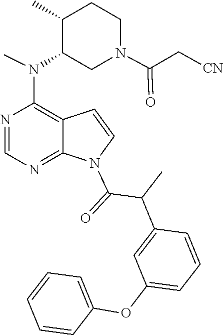 [SMILES: CC(C(=O)n1ccc2c(N(C)[C@H]3CN(C(=O)CC#N)CC[C@H]3C)ncnc21)c1cccc(Oc2ccccc2)c1] | 3-((3R,4R)-4-methyl-3-(methyl (7-(2-(3-phenoxyphenyl) propanoyl)-7H-pyrrolo [2,3-d]pyrimidin-4-yl) amino)piperidin-1-yl)-3-oxopropanenitrile |
| 11 (CPD-011) | 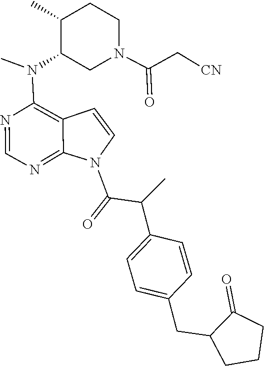 [SMILES: CC(C(=O)n1ccc2c(N(C)[C@H]3CN(C(=O)CC#N)CC[C@H]3C)ncnc21)c1ccc(CC2CCCC2=O)cc1] | 3-((3R,4R)-4-methyl-3-(methyl (7-(2-(4-((2-oxocyclopentyl)methyl)phenyl) propanoyl)-7H-pyrrolo[2,3-d]pyrimidin-4-yl) amino)piperidin-1-yl)-3-oxopropanenitrile |
| 12 (CPD-012) | 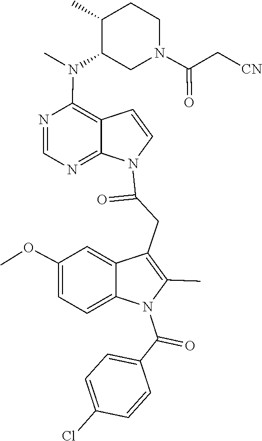 [SMILES: COc1ccc2c(c1)c(CC(=O)n1ccc3c(N(C)[C@H]4CN(C(=O)CC#N)CC[C@H]4C)ncnc31)c(C)n2C(=O)c1ccc(Cl)cc1] | 3-((3R, 4R)-3-((7-(2-(1-(4-chlorobenzoyl)-5-methoxy- 2-methyl-1H-indol-3-yl)acetyl)-7H-pyrrolo[2, 3-d]pyrimidin-4-yl)(methyl) amino)-4-methylpiperidin-1-yl)-3- oxopropanenitrile |
| 13 (CPD-013) | 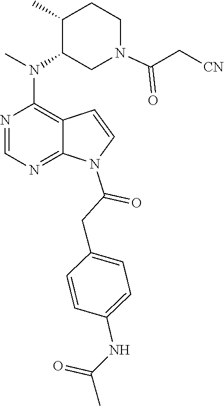 [SMILES: CC(=O)Nc1ccc(CC(=O)n2ccc3c(N(C)[C@H]4CN(C(=O)CC#N)CC[C@H]4C)ncnc32)cc1] | N-(4-(2-(4-(((3R, 4R)-1-(2-cyanoacetyl)-4-methylpiperidin-3-yl) (methyl)amino)-7H-pyrrolo[2,3-d] pyrimidin-7-yl)-2-oxoethyl)phenyl)acetamide |
| 14 (CPD-014) | 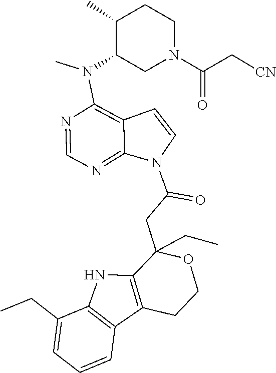 [SMILES: CCc1cccc2c3c([nH]c12)C(CC)(CC(=O)n1ccc2c(N(C)[C@H]4CN(C(=O)CC#N)CC[C@H]4C)ncnc21)OCC3] | 3-((3R,4R)-3-((7-(2-(1,8-diethyl-1,3,4, 9-tetrahydropyrano[3,4-b]indol-1-yl) acetyl)-7H-pyrrolo[2,3-d]pyrimidin-4-yl) (methyl) amino)-4-methylpiperidin-1-yl)-3- oxopropanenitrile |
| 15 (CPD-015) | 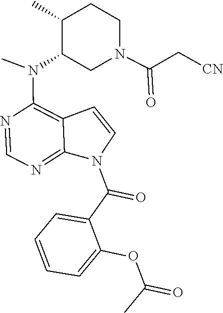 [SMILES: CC(=O)Oc1ccccc1C(=O)n1ccc2c(N(C)[C@H]3CN(C(=O)CC#N)CC[C@H]3C)ncnc21] | 2-(4-((3R, 4R)-1-(2-cyanoacetyl)-4-methylpiperidin-3-yl) (methyl)amino)-7H-pyrrolo[2,3-d] pyrimidine-7-carbonyl)phenyl acetate |
| 16 (CPD-016) | 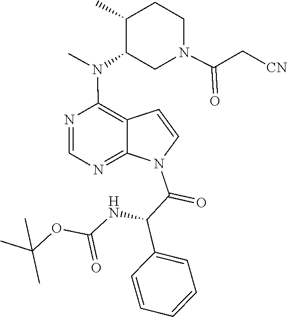 [SMILES: C[C@@H]1CCN(C(=O)CC#N)C[C@@H]1N(C)c1ncnc2c1ccn2C(=O)[C@@H](NC(=O)OC(C)(C)C)c1ccccc1] | Tert-butyl ((S)-2-(4-(((3R, 4R)-1-(2-cyanoacetyl)-4-methylpiperidin-3-yl) (methyl)amino)-7H-pyrrolo[2,3-d]pyrimidin- 7-yl)-2-oxo-1-phenylethyl)carbamate |
| 17 (CPD-017) | 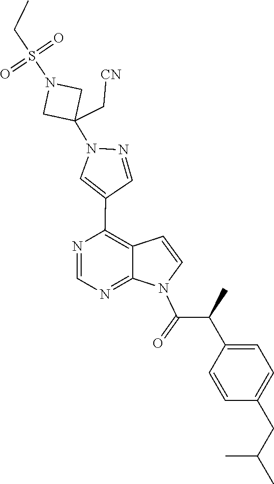 [SMILES: CCS(=O)(=O)N1CC(CC#N)(n2cc(-c3ncnc4c3ccn4C(=O)[C@@H](C)c3ccc(CC(C)C)cc3)cn2)C1] | (S)-2-(1-(ethylsulfonyl)- 3-(4-(7-(2-(4-isobutyl- phenyl)propanoyl)-7H-pyrrolo[2,3-d] pyrimidin-4-yl)-1H-pyrazol-1-yl)azetidin- 3-yl)acetonitrile |
| 18 (CPD-018) | 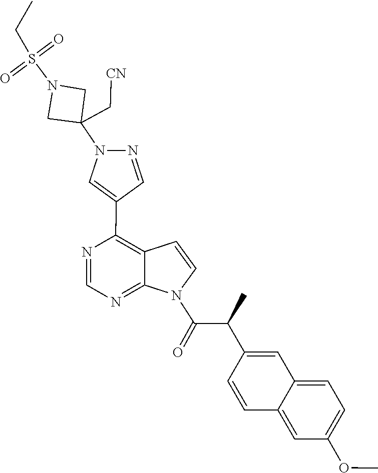 [SMILES: CCS(=O)(=O)N1CC(CC#N)(n2cc(-c3ncnc4c3ccn4C(=O)[C@@H](C)c3ccc4cc(OC)ccc4c3)cn2)C1] | (S)-2-(1-(ethylsulfonyl)- 3-(4-(7-(2-(6-methoxy naphthalen-2-yl)propanoyl)-7H-pyrrolo[2, 3-d]pyrimidin-4-yl)-1H-pyrazol-1-yl) azetidin-3-yl)acetonitrile |
| 19 (CPD-019) | 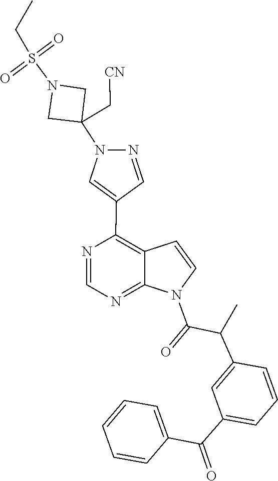 [SMILES: CCS(=O)(=O)N1CC(CC#N)(n2cc(-c3ncnc4c3ccn4C(=O)C(C)c3cccc(C(=O)c4ccccc4)c3)cn2)C1] | 2-(3-(4-(7-(2-(3-benzoylphenyl) propanoyl)-7H-pyrrolo[2,3-d] pyrimidin-4-yl)-1H-pyrazol-1-yl)-1- (ethylsulfonyl)azetidin-3-yl)acetonitrile |
| 20 (CPD-020) | 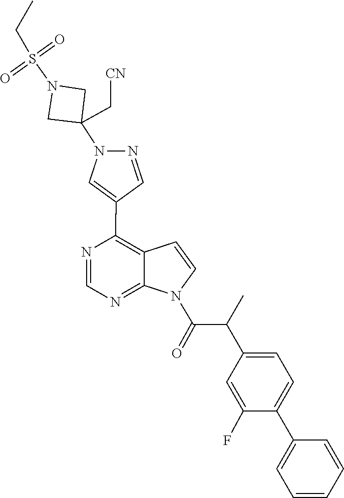 [SMILES: CCS(=O)(=O)N1CC(CC#N)(n2cc(-c3ncnc4c3ccn4C(=O)C(C)c3ccc(-c4ccccc4)c(F)c3)cn2)C1] | 2-(1-(ethylsulfonyl)-3-(4-(7-(2-(2-fluoro-[1, 1′-biphenyl]-4-yl)propanoyl)-7H-pyrrolo[2, 3-d]pyrimidin-4-yl)-1H-pyrazol-1-yl) azetidin-3-yl)acetonitrile |
| 21 (CPD-021) | 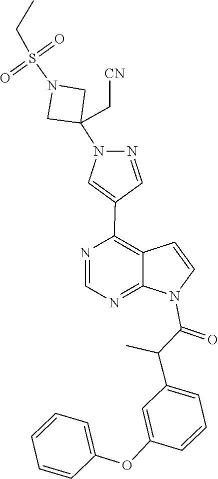 [SMILES: CCS(=O)(=O)N1CC(CC#N)(n2cc(-c3ncnc4c3ccn4C(=O)C(C)c3cccc(Oc4ccccc4)c3)cn2)C1] | 2-(1-(ethylsulfonyl)-3-(4-(7-(2-(3-phenoxy- phenyl)propanoyl)-7H- pyrrolo[2,3-d]pyrimidin- 4-yl)-1H-pyrazol-1-yl)azetidin-3-yl) acetonitrile |
| 22 (CPD-022) | 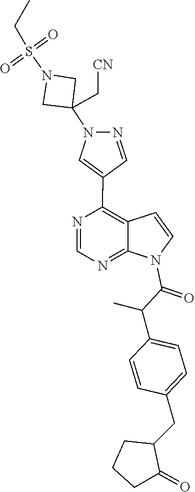 [SMILES: CCS(=O)(=O)N1CC(CC#N)(n2cc(-c3ncnc4c3ccn4C(=O)C(C)c3ccc(CC4CCCC4=O)cc3)cn2)C1] | 2-(1-(ethylsulfonyl)-3- (4-(7-(2-(4-(2-oxocyclo- pentyl)methyl)phenyl)propanoyl)-7H-pyrrolo [2,3-d]pyrimidin-4-yl)-1H-pyrazol-1-yl) azetidin-3-yl)acetonitrile |
| 23 (CPD-023) | 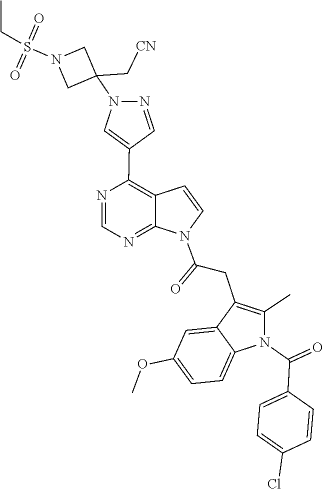 [SMILES: CCS(=O)(=O)N1CC(CC#N)(n2cc(-c3ncnc4c3ccn4C(=O)Cc3c(C)n(C(=O)c4ccc(Cl)cc4)c4ccc(OC)cc34)cn2)C1] | 2-(3-(4-(7-(2-(1-(4- chlorobenzoyl)-5-methoxy- 2-methyl-1H-indol-3-yl)acetyl)-7H-pyrrolo [2,3-d]pyrimidin- 4-yl)-1H-pyrazol-1-yl)-1-(ethylsulfonyl) azetidin-3-yl)acetonitrile |
| 24 (CPD-024) | 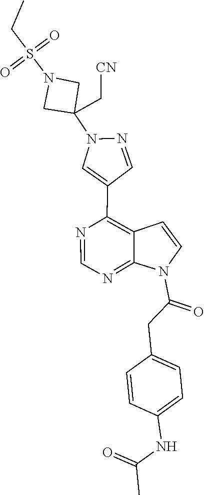 [SMILES: CCS(=O)(=O)N1CC(CC#N)(n2cc(-c3ncnc4c3ccn4C(=O)Cc3ccc(NC(C)=O)cc3)cn2)C1] | N-(4-(2-(4-(1-(3- (cyanomethyl)-1-(ethylsulfonyl) azetidin-3-yl)-1H-pyrazol-4-yl)-7H-pyrrolo [2,3-d]pyrimidin-7-yl)-2-oxoethyl)phenyl) acetamide |
| 25 (CPD-025) | 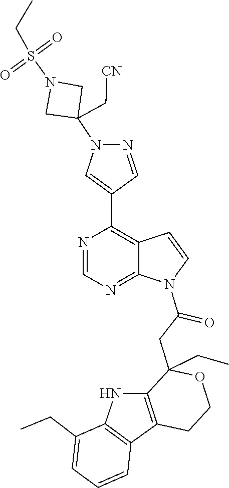 [SMILES: CCc1cccc2c3c([nH]c12)C(CC)(CC(=O)n1ccc2c(-c4cnn(C5(CC#N)CN(S(=O)(=O)CC)C5)c4)ncnc21)OCC3] | 2-(3-(4-(7-(2-(1,8-diethyl-1,3,4, 9-tetrahydropyrano[3,4-b]indol-1-yl) acetyl)-7H-pyrrolo[2,3-d]pyrimidin- 4-yl)-1H-pyrazol-1-yl)-1-(ethylsulfonyl) azetidin-3-yl)acetonitrile |
| 26 (CPD-026) | 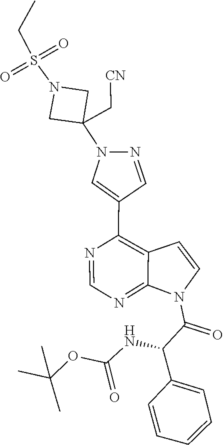 [SMILES: CCS(=O)(=O)N1CC(CC#N)(n2cc(-c3ncnc4c3ccn4C(=O)[C@@H](NC(=O)OC(C)(C)C)c3ccccc3)cn2)C1] | Tert-butyl (S)-(2-(4-(1-(3-(cyanomethyl)- 1-(ethylsulfonyl) azetidin-3-yl)-1H-pyrazol-4-yl)-7H-pyrrolo[2, 3-d]pyrimidin-7-yl)-2-oxo-1-phenylethyl) carbamate |
| 27 (CPD-027) | 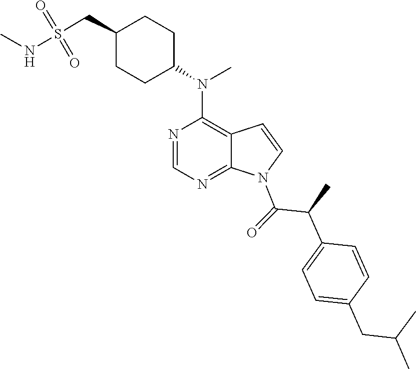 | 1-((Trans-)-4-((7-((S)-2-(4-isobutylphenyl) propanoyl)-7H-pyrrolo[2,3-d]pyrimidin-4-yl) (methyl)amino)cyclohexyl)-N-methyl methanesulfonamide |
| 28 (CPD-028) | 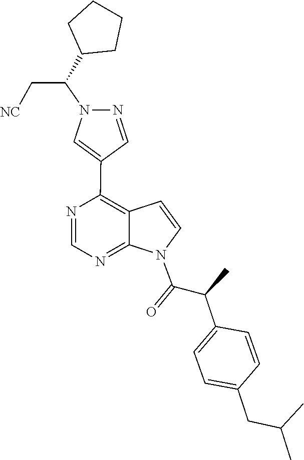 [SMILES: CC(C)Cc1ccc([C@H](C)C(=O)n2ccc3c(-c4cnn([C@H](CC#N)C5CCCC5)c4)ncnc32)cc1] | (R)-3-cyclopentyl-3-(4-(7- (S)-2-(4-isobutylphenyl) propanoyl)-7H-pyrrolo[2,3-d]pyrimidin- 4-yl)-1H-pyrazol-1-yl)propanenitrile |
| 29 (CPD-029) | 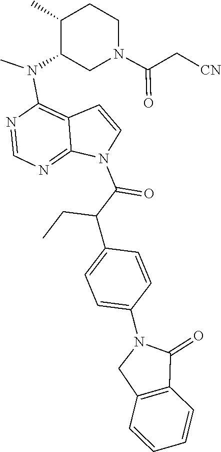 [SMILES: CCC(C(=O)n1ccc2c(N(C)[C@H]3CN(C(=O)CC#N)CC[C@H]3C)ncnc21)c1ccc(N2Cc3ccccc3C2=O)cc1] | 3-((3R,4R)-4-methyl-3-(methyl (7-(2-(4-(1-oxoisoindolin-2-yl)phenyl) butanoyl)-7H-pyrrolo[2,3-d]pyrimidin-4-yl) amino)piperidin-1-yl)-3-oxopropanenitrile |
| 30 (CPD-030) | 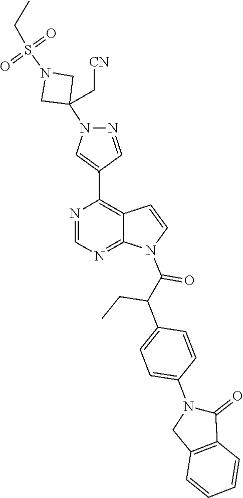 [SMILES: CCC(C(=O)n1ccc2c(-c3cnn(C4(CC#N)CN(S(=O)(=O)CC)C4)c3)ncnc21)c1ccc(N2Cc3ccccc3C2=O)cc1] | 2-2-(1-(Ethylsulfonyl)- 3-(4-(7-(2-(4-(1-oxoiso- indolin-2-yl)phenyl)butanoyl)-7H-pyrrolo[2, 3-d]pyrimidin-4-yl)-1H- pyrazol-1-yl)azetidin- 3-yl)acetonitrile |
| 31 (CPD-039) | 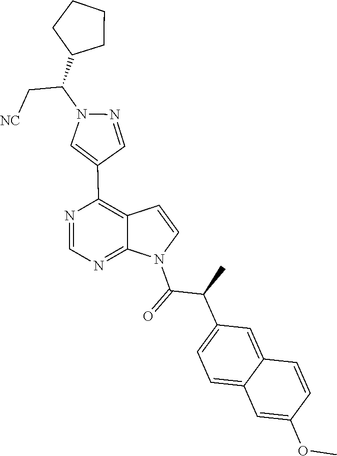 [SMILES: COc1ccc2cc([C@H](C)C(=O)n3ccc4c(-c5cnn([C@H](CC#N)C6CCCC6)c5)ncnc43)ccc2c1] | (R)-3-cyclopentyl-3-(4- (7-((S)-2-(6-methoxy- naphthalen-2-yl)propanoyl)- 7H-pyrrolo[2,3-d] pyrimidin-4-yl))-1H-pyrazol-1-yl) propanenitrile |
| 32 (CPD-040) | 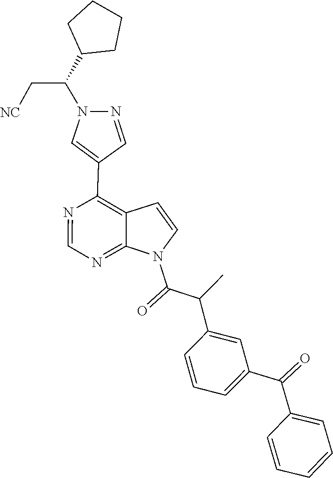 [SMILES: CC(C(=O)n1ccc2c(-c3cnn([C@H](CC#N)C4CCCC4)c3)ncnc21)c1cccc(C(=O)c2ccccc2)c1] | (3R)-3-(4-(7-(2-(3-benzoylphenyl) propanoyl)-7H-pyrrolo[2,3-d]pyrimidin- 4-yl)-1H-pyrazol-1-yl)-3- cyclopentylpropanenitrile |
| 33 (CPD-041) | 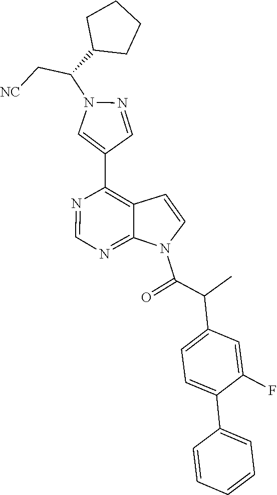 [SMILES: CC(C(=O)n1ccc2c(-c3cnn([C@H](CC#N)C4CCCC4)c3)ncnc21)c1ccc(-c2ccccc2)c(F)c1] | (3R)-3-cyclopentyl-3-(4-(7-(2-(2-fluoro-[1, 1′-biphenyl]-4-yl)propanoyl)-7H-pyrrolo[2, 3-d]pyrimidin-4-yl)-1H-pyrazol-1-yl) propanenitrile |
| 34 (CPD-050) | 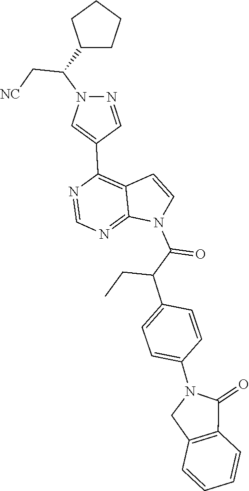 [SMILES: CCC(C(=O)n1ccc2c(-c3cnn([C@H](CC#N)C4CCCC4)c3)ncnc21)c1ccc(N2Cc3ccccc3C2=O)cc1] | (3R)-3-cyclopentyl-3-(4- (7-(2-(4-(1-oxoisoindolin- 2-yl)phenyl)butanoyl)-7H-pyrrolo[2,3-d] pyrimidin-4-yl)-1H-pyrazol-1-yl) propanenitrile |
| 35 (CPD-065) | 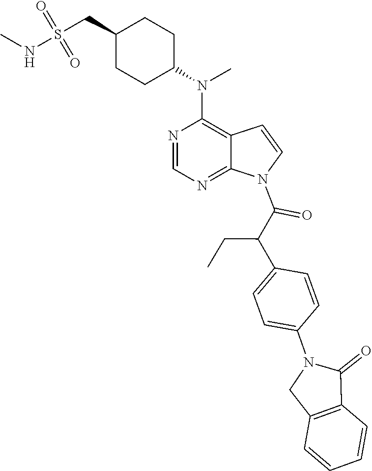 | N-methyl-1-((trans)-4-(methyl (7-(2-(4-(1-oxoisoindolin-2-yl)phenyl) butanoyl)-7H-pyrrolo[2,3-d]pyrimidin-4-yl) amino)cyclohexyl)methanesulfonamide |
| 36 (CPD-056) | 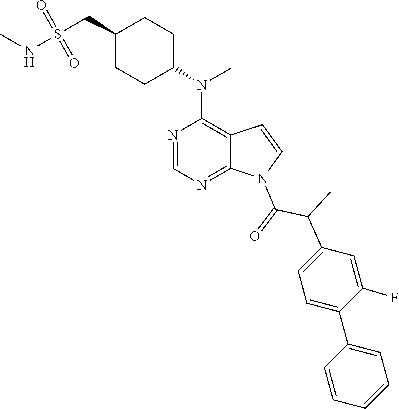 | 1-((Trans)-4-((7-(2-(2-fluoro-[1, 1′-biphenyl]-4-yl)propanoyl)-7H-pyrrolo[2, 3-d]pyrimidin-4-yl)(methyl)amino) cyclohexyl)-N-methyl methanesulfonamide |
| 37 (CPD-062) | 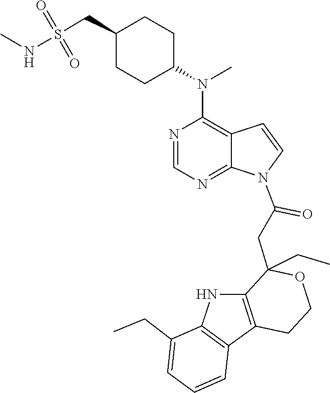 | 1-((Trans)-4-((7-(2-(1,8-diethyl-1,3,4, 9-tetrahydropyrano[3,4-b]indol-1-yl) acetyl)-7H-pyrrolo[2, 3-d]pyrimidin-4-yl) (methyl)amino)cyclohexyl)-N-methyl methanesulfonamide |
| 38 (CPD-055) | 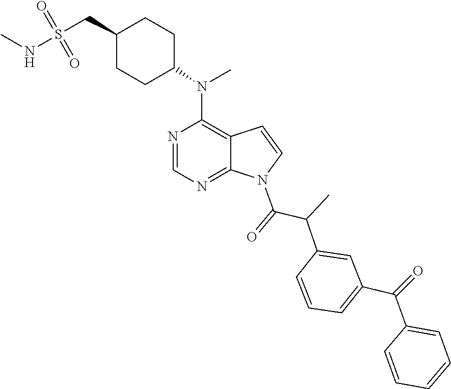 | 1-((Trans)-4-((7-(2-(3-benzoylphenyl) propanoyl)-7H-pyrrolo[2,3-d]pyrimidin-4-yl) (methyl)amino)cyclohexyl)-N-methyl methanesulfonamide |
| 39 (CPD-054) | 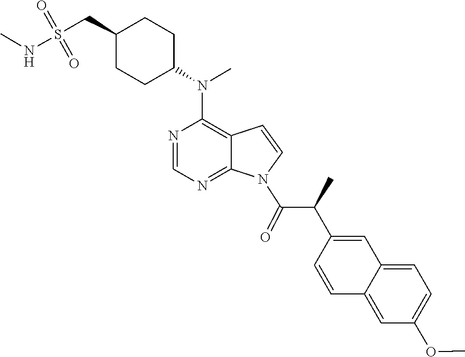 | 1-((Trans)-4-((7-((S)- 2-(6-methoxynaphthalen- 2-yl)propanoyl)-7H-pyrrolo[2,3-d]pyrimidin- 4-yl)(methyl)amino)cyclohexyl)-N-methyl methanesulfonamide |
| 40 (CPD-032) | 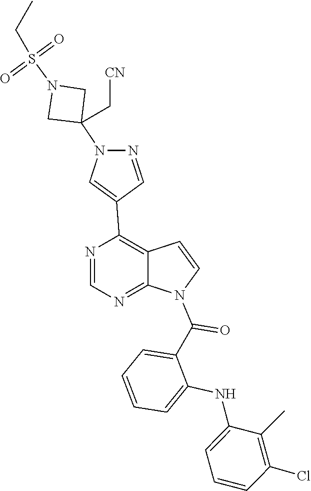 [SMILES: CCS(=O)(=O)N1CC(CC#N)(n2cc(-c3ncnc4c3ccn4C(=O)c3ccccc3Nc3cccc(Cl)c3C)cn2)C1] | 2-(3-(4-(7-(2-((3-Chloro-2-methylphenyl) amino)benzoyl)-7H- pyrrolo[2,3-d]pyrimidin- 4-yl)-1H-pyrazol-1-yl)-1-(ethylsulfonyl) azetidin-3-yl)acetonitrile |
| 41 (CPD-053) | 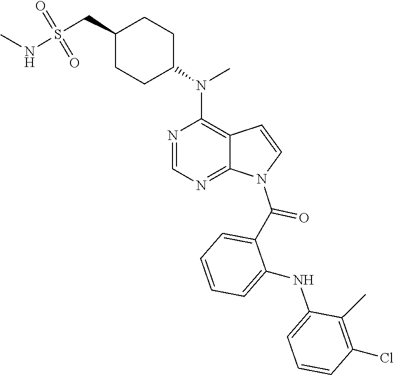 | 1-((Trans)-4-((7-(2-((3- chloro-2-methylphenyl) amino)benzoyl)-7H-pyrrolo[2,3-d]pyrimidin- 4-yl)(methyl)amino)cyclohexyl)-N-methyl methanesulfonamide |
| 42 (CPD-060) | 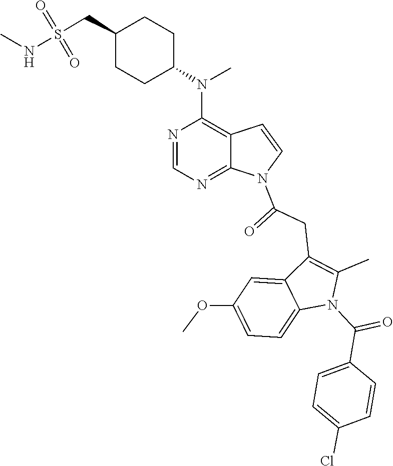 [SMILES: CNS(=O)(=O)C[C@H]1CC[C@H](N(C)c2ncnc3c2ccn3C(=O)Cc2c(C)n(C(=O)c3ccc(Cl)cc3)c3ccc(OC)cc23)CC1] | 1-((Trans)-4-((7-(2-(1-(4-chlorobenzoyl)-5- methoxy-2-methyl-1H-indol-3-yl) acetyl)-7H-pyrrolo[2,3-d]pyrimidin-4-yl) (methyl)amino) cyclohexyl)-N-methylmethanesulfonamide |
| 43 (CPD-052) | 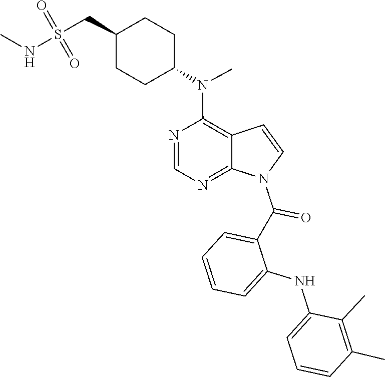 | 1-((Trans)-4-((7-(2-((2,3-dimethylphenyl) amino)benzoyl)-7H-pyrrolo[2,3-d]pyrimidin- 4-yl)(methyl)amino)cyclohexyl)-N-methyl methanesulfonamide |
| 44 (CPD-059) | 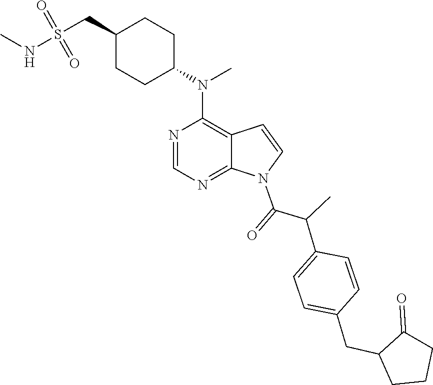 [SMILES: CNS(=O)(=O)C[C@H]1CC[C@H](N(C)c2ncnc3c2ccn3C(=O)C(C)c2ccc(CC3CCCC3=O)cc2)CC1] | N-methyl-1-((trans)-4-(methyl (7-(2-(4-((2-oxocyclopentyl)methyl)phenyl) propanoyl)-7H-pyrrolo[2,3-d]pyrimidin-4-yl) amino)cyclohexyl)methanesulfonamide |
| 45 (CPD-047) | 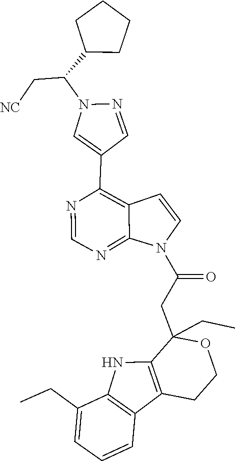 [SMILES: CCc1cccc2c3c([nH]c12)C(CC)(CC(=O)n1ccc2c(-c4cnn([C@H](CC#N)C5CCCC5)c4)ncnc21)OCC3] | (3R)-3-cyclopentyl-3-(4-(7-(2-(1,8-diethyl-1, 3,4,9-tetrahydropyran[3,4-b]indol-1-yl) acetyl)-7H-pyrrolo[2,3-d]pyrimidin- 4-yl)-1H-pyrazol-1-yl)propanenitrile |
| 46 (CPD-064) | 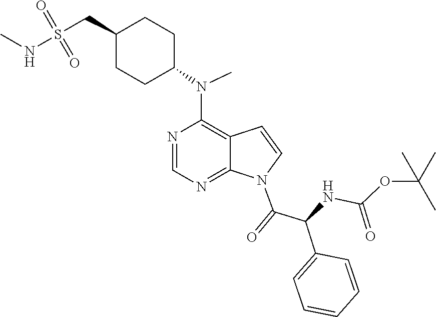 [SMILES: CNS(=O)(=O)C[C@H]1CC[C@H](N(C)c2ncnc3c2ccn3C(=O)[C@@H](NC(=O)OC(C)(C)C)c2ccccc2)CC1] | Tert-butyl ((S)-2-(4-(methyl ((trans)-4-((N-methylsulfamoyl)methyl) cyclohexyl)amino)-7H-pyrrolo[2,3-d] pyrimidin-7-yl)-2-oxo-1-phenylethyl) carbamate |
| 47 (CPD-031) | 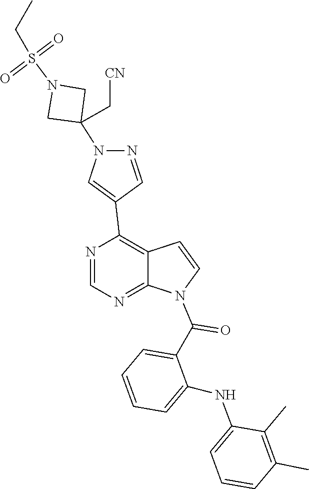 [SMILES: CCS(=O)(=O)N1CC(CC#N)(n2cc(-c3ncnc4c3ccn4C(=O)c3ccccc3Nc3cccc(C)c3C)cn2)C1] | 2-(3-(4-(7-(2-((2,3-Dimethylphenyl)amino) benzoyl)-7H-pyrrolo[2,3-d]pyrimidin- 4-yl)-1H-pyrazol-1-yl)-1-(ethylsulfonyl) azetidin-3-yl)acetonitrile |
| 48 (CPD-082) | 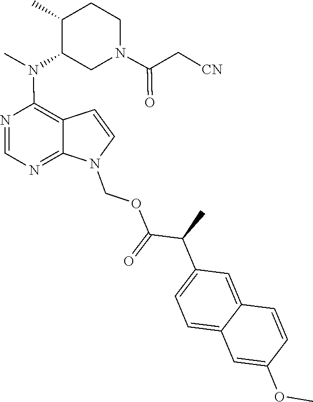 | 4-(((3R, 4R)-1-(2-cyanoacetyl)- 4-methylpiperidin-3-yl) (methyl)amino)-7H-pyrrolo[2,3-d]pyrimidin- 7-yl)methyl (S)-2-(6-methoxynaphthalen-2-yl)propanoate |
| 49 (CPD-083) | 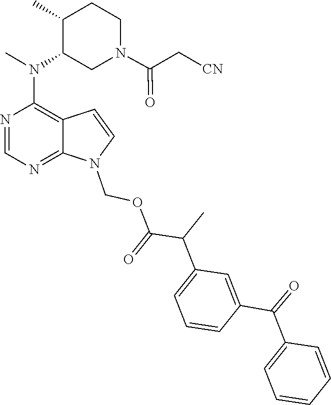 [SMILES: CC(C(=O)OCn1ccc2c(N(C)[C@H]3CN(C(=O)CC#N)CC[C@H]3C)ncnc21)c1cccc(C(=O)c2ccccc2)c1] | (4-(((3R, 4R)-1-(2-cyanoacetyl)- 4-methylpiperidin-3-yl) (methyl)amino)-7H-pyrrolo[2,3-d]pyrimidin- 7-yl)methyl 2-(3-benzoylphenyl)propanoate |
| 50 (CPD-084) | 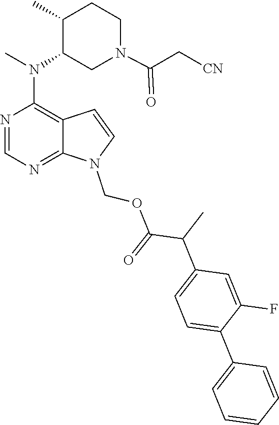 [SMILES: CC(C(=O)OCn1ccc2c(N(C)[C@H]3CN(C(=O)CC#N)CC[C@H]3C)ncnc21)c1ccc(-c2ccccc2)c(F)c1] | (4-(((3R, 4R)-1-(2-cyanoacetyl)- 4-methylpiperidin-3-yl) (methyl)amino)-7H-pyrrolo[2,3-d]pyrimidin- 7-yl)methyl 2-(2-fluoro-[1,1′-biphenyl]-4-yl) propanoate |
| 51 (CPD-085) | 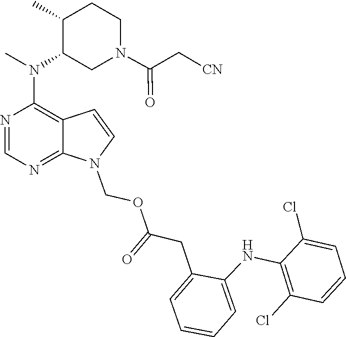 [SMILES: C[C@@H]1CCN(C(=O)CC#N)C[C@@H]1N(C)c1ncnc2c1ccn2COC(=O)Cc1ccccc1Nc1c(Cl)cccc1Cl] | (4-(((3R, 4R)-1-(2-cyanoacetyl)- 4-methylpiperidin-3-yl) (methyl)amino)-7H-pyrrolo[2,3-d]pyrimidin- 7-yl)methyl 2-(2-((2,6-dichlorophenyl) amino)phenyl)acetate |
| 52 (CPD-086) | 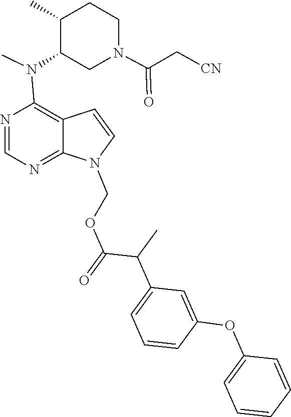 [SMILES: CC(C(=O)OCn1ccc2c(N(C)[C@H]3CN(C(=O)CC#N)CC[C@H]3C)ncnc21)c1cccc(Oc2ccccc2)c1] | (4-(((3R, 4R)-1-(2-cyanoacetyl)- 4-methylpiperidin-3-yl) (methyl)amino)-7H-pyrrolo[2,3-d]pyrimidin- 7-yl)methyl 2-(3-phenoxyphenyl)propanoate |
| 53 (CPD-087) | 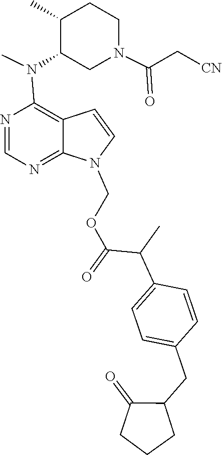 [SMILES: CC(C(=O)OCn1ccc2c(N(C)[C@H]3CN(C(=O)CC#N)CC[C@H]3C)ncnc21)c1ccc(CC2CCCC2=O)cc1] | (4-(((3R,4R)- 1-(2-cyanoacetyl)-4-methylpiperidin-3-yl) (methyl)amino)-7H-pyrrolo[2,3-d]pyrimidin- 7-yl)methyl 2-(4-((2-oxocyclopentyl)methyl) phenyl)propanoate |
| 54 (CPD-043) | 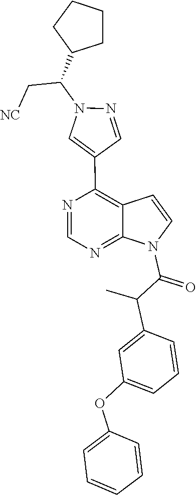 [SMILES: CC(C(=O)n1ccc2c(-c3cnn([C@H](CC#N)C4CCCC4)c3)ncnc21)c1cccc(Oc2ccccc2)c1] | (3R)-3-cyclopentyl-3-(4- (7-(2-(3-phenoxyphenyl) propanoyl)-7H-pyrrolo[2,3-d]pyrimidin- 4-yl)-1H-pyrazol-1-yl)propanenitrile |
| 55 (CPD-088) | 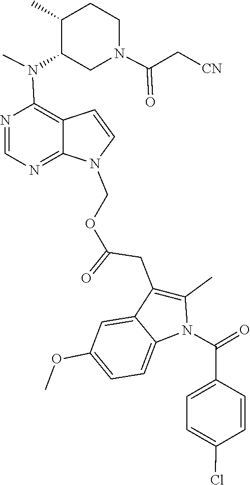 [SMILES: COc1ccc2c(c1)c(CC(=O)OCn1ccc3c(N(C)[C@H]4CN(C(=O)CC#N)CC[C@H]4C)ncnc31)c(C)n2C(=O)c1ccc(Cl)cc1] | (4-(((3R, 4R)-1-(2-cyanoacetyl)- 4-methylpiperidin-3-yl) (methyl)amino)-7H-pyrrolo[2,3-d]pyrimidin- 7-yl)methyl 2-(1-(4-chlorobenzoyl)-5-methoxy-2-methyl- 1H-indol-3-yl)acetate |
| 56 (CPD-089) | 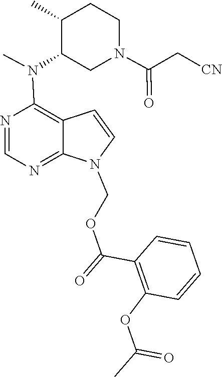 [SMILES: CC(=O)Oc1ccccc1C(=O)OCn1ccc2c(N(C)[C@H]3CN(C(=O)CC#N)CC[C@H]3C)ncnc21] | (4-(((3R, 4R)-1-(2-cyanoacetyl)- 4-methylpiperidin-3-yl) (methyl)amino)-7H-pyrrolo[2,3-d]pyrimidin- 7-yl)methyl 2-acetoxybenzoate |
| 57 (CPD-038) | 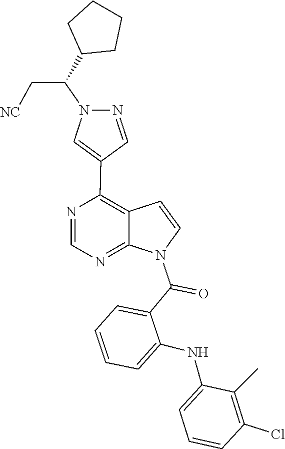 [SMILES: Cc1c(Cl)cccc1Nc1ccccc1C(=O)n1ccc2c(-c3cnn([C@H](CC#N)C4CCCC4)c3)ncnc21] | (R)-3-(4-(7-(2-((3-chloro-2-methylphenyl) amino)benzoyl)-7H-pyrrolo[2,3-d]pyrimidin- 4-yl)-1H-pyrazol-1-yl)-3- cyclopentylpropanenitrile |
| 58 (CPD-061) | 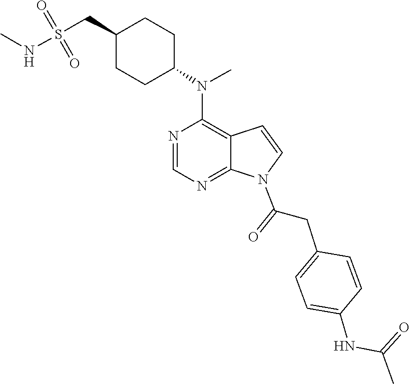 [SMILES: CNS(=O)(=O)C[C@H]1CC[C@H](N(C)c2ncnc3c2ccn3C(=O)Cc2ccc(NC(C)=O)cc2)CC1] | N-(4-(2-(4-(methyl ((trans)-4-((N-methylsulfamoyl)methyl) cyclohexyl)amino)-7H-pyrrolo[2,3-d] pyrimidin-7-yl)-2-oxoethyl)phenyl)acetamide |
| 59 (CPD-140) | 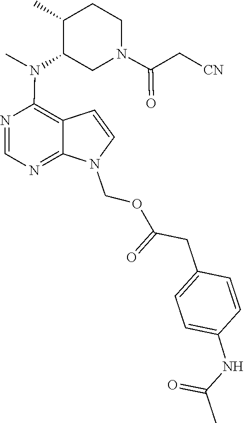 [SMILES: CC(=O)Nc1ccc(CC(=O)OCn2ccc3c(N(C)[C@H]4CN(C(=O)CC#N)CC[C@H]4C)ncnc32)cc1] | (4-(((3R, 4R)-1-(2-cyanoacetyl)- 4-methylpiperidin-3-yl) (methyl)amino)-7H-pyrrolo[2,3-d]pyrimidin- 7-yl)methyl 2-(4-acetamidophenyl)acetate |
| 60 (CPD-092) | 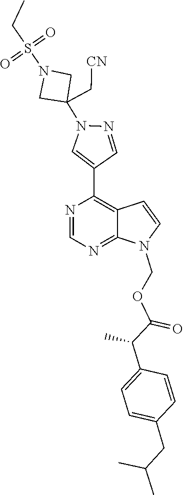 [SMILES: CCS(=O)(=O)N1CC(CC#N)(n2cc(-c3ncnc4c3ccn4COC(=O)[C@@H](C)c3ccc(CC(C)C)cc3)cn2)C1] | Methyl (4-(1-(3-(cyanomethyl)-1-(ethylsulfonyl) azetidin-3-yl)-1H- pyrazol-4-yl)-7H-pyrrolo[2, 3-d]pyrimidin-7-yl) (S)-2-(4-isobutylphenyl) propanoate |
| 61 (CPD-093) | 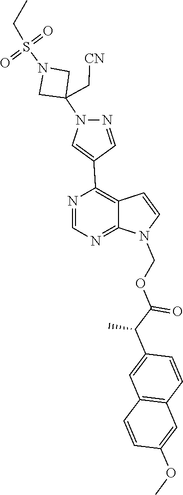 [SMILES: CCS(=O)(=O)N1CC(CC#N)(n2cc(-c3ncnc4c3ccn4COC(=O)[C@@H](C)c3ccc4cc(OC)ccc4c3)cn2)C1] | Methyl (4-(1-(3-(cyanomethyl)-1-(ethylsulfonyl) azetidin-3-yl)-1H-pyrazol- 4-yl)-7H-pyrrolo[2, 3-d]pyrimidin-7-yl) (S)-2-(6-methoxynaphthalen-2-yl)propanoate |
| 62 (CPD-094) | 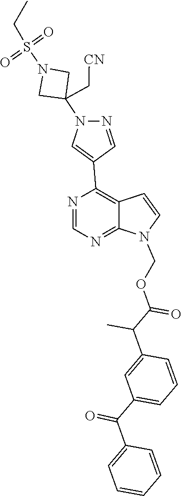 [SMILES: CCS(=O)(=O)N1CC(CC#N)(n2cc(-c3ncnc4c3ccn4COC(=O)C(C)c3cccc(C(=O)c4ccccc4)c3)cn2)C1] | 4-(1-(3-(cyanomethyl)-1-(ethylsulfonyl) azetidin-3-yl)-1H-pyrazol- 4-yl)-7H-pyrrolo[2, 3-d]pyrimidin-7-yl)methyl 2-(3-benzoylphenyl)propanoate |
| 63 (CPD-139) | 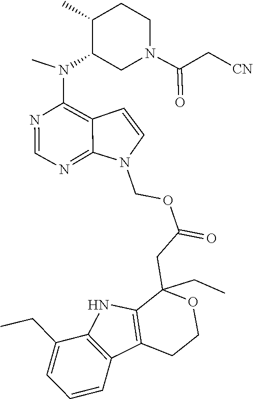 [SMILES: CCc1cccc2c3c([nH]c12)C(CC)(CC(=O)OCn1ccc2c(N(C)[C@H]4CN(C(=O)CC#N)CC[C@H]4C)ncnc21)OCC3] | (4-(((3R, 4R)-1-(2-cyanoacetyl)- 4-methylpiperidin-3-yl) (methyl)amino)-7H-pyrrolo[2,3-d]pyrimidin- 7-yl)methyl 2-(1,8-diethyl-1,3,4, 9-tetrahydropyrano[3,4-b]indol-1-yl)acetate |
| 64 (CPD-141) | 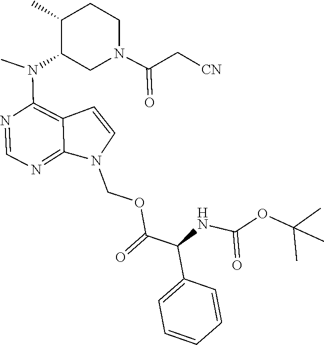 [SMILES: C[C@@H]1CCN(C(=O)CC#N)C[C@@H]1N(C)c1ncnc2c1ccn2COC(=O)[C@@H](NC(=O)OC(C)(C)C)c1ccccc1] | (4-(((3R, 4R)-1-(2-cyanoacetyl)- 4-methylpiperidin-3-yl) (methyl)amino)-7H-pyrrolo[2,3-d]pyrimidin- 7-yl)methyl (S)-2-((tert-butoxycarbonyl) amino)-2-phenylacetate |
| 65 (CPD-136) | 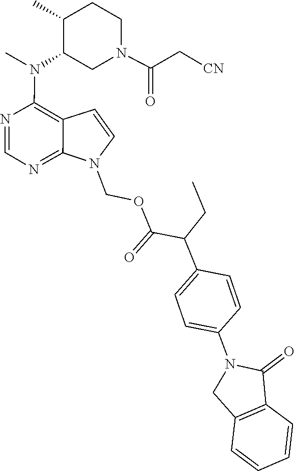 [SMILES: CCC(C(=O)OCn1ccc2c(N(C)[C@H]3CN(C(=O)CC#N)CC[C@H]3C)ncnc21)c1ccc(N2Cc3ccccc3C2=O)cc1] | (4-(((3R, 4R)-1-(2-cyanoacetyl)- 4-methylpiperidin-3-yl) (methyl)amino)-7H-pyrrolo[2,3-d]pyrimidin- 7-yl)methyl 2-(4-(1-oxoisoindol-2-yl)) phenyl)butanoate |
| 66 (CPD-138) | 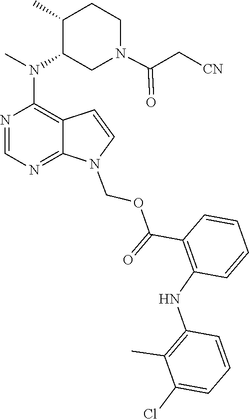 [SMILES: Cc1c(Cl)cccc1Nc1ccccc1C(=O)OCn1ccc2c(N(C)[C@H]3CN(C(=O)CC#N)CC[C@H]3C)ncnc21] | (4-(((3R, 4R)-1-(2-cyanoacetyl)- 4-methylpiperidin-3-yl) (methyl)amino)-7H-pyrrolo[2,3-d]pyrimidin- 7-yl)methyl 2-((3-chloro-2-methylphenyl) amino)benzoate |
| 67 (CPD-137) | 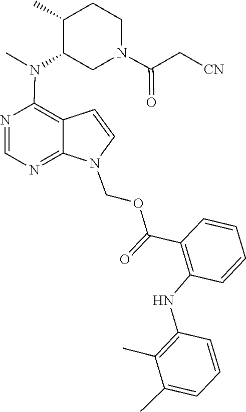 [SMILES: Cc1cccc(Nc2ccccc2C(=O)OCn2ccc3c(N(C)[C@H]4CN(C(=O)CC#N)CC[C@H]4C)ncnc32)c1C] | (4-(((3R, 4R)-1-(2-cyanoacetyl)- 4-methylpiperidin-3-yl) (methyl)amino)-7H-pyrrolo[2,3-d]pyrimidin- 7-yl)methyl 2-((2,3-dimethylphenyl)amino) benzoate |
| 68 (CPD-058) | 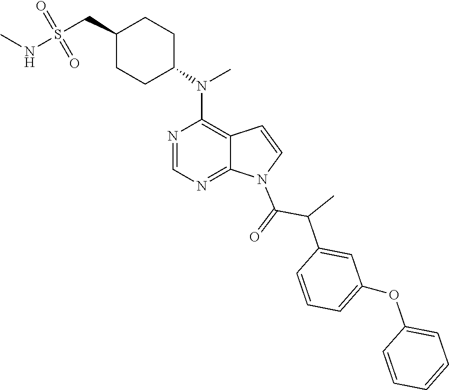 [SMILES: CNS(=O)(=O)C[C@H]1CC[C@H](N(C)c2ncnc3c2ccn3C(=O)C(C)c2cccc(Oc3ccccc3)c2)CC1] | N-methyl-1-((trans)-4-(methyl (7-(2-(3-phenoxyphenyl) propanoyl)-7H-pyrrolo[2,3-d]pyrimidin-4-yl) amino)cyclohexyl)methanesulfonamide |
| 69 (CPD-095) | 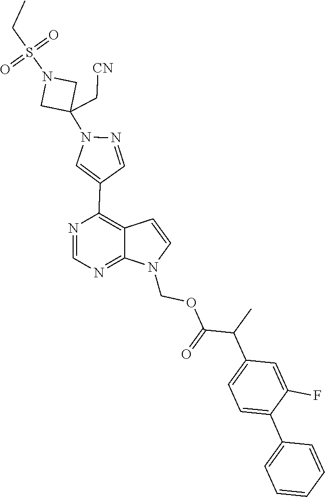 [SMILES: CCS(=O)(=O)N1CC(CC#N)(n2cc(-c3ncnc4c3ccn4COC(=O)C(C)c3ccc(-c4ccccc4)c(F)c3)cn2)C1] | Methyl (4-(1-(3-(cyanomethyl)-1-(ethylsulfonyl) azetidin-3-yl)-1H-pyrazol-4-yl)-7H-pyrrolo[2, 3-d]pyrimidin-7-yl) 2-(2-fluoro-[1, 1′-biphenyl]-4-yl)propanoate |
| 70 (CPD-096) | 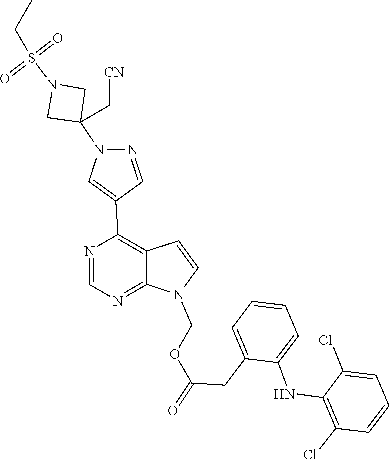 [SMILES: CCS(=O)(=O)N1CC(CC#N)(n2cc(-c3ncnc4c3ccn4COC(=O)Cc3ccccc3Nc3c(Cl)cccc3Cl)cn2)C1] | Methyl (4-(1-(3-(cyanomethyl)-1-(ethylsulfonyl) azetidin-3-yl)-1H-pyrazol-4-yl)-7H-pyrrolo[2, 3-d]pyrimidin-7-yl) 2-(2-((2, 6-dichlorophenyl)amino)phenyl)acetate |
| 71 (CPD-097) | 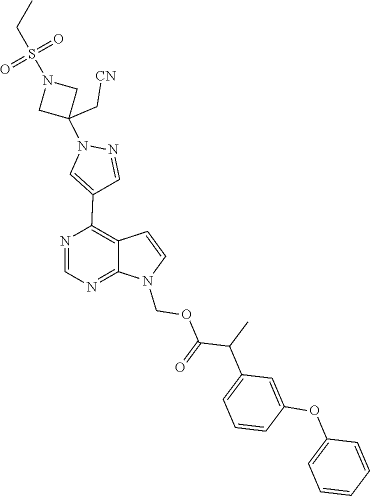 [SMILES: CCS(=O)(=O)N1CC(CC#N)(n2cc(-c3ncnc4c3ccn4COC(=O)C(C)c3cccc(Oc4ccccc4)c3)cn2)C1] | Methyl (4-(1-(3-(cyanomethyl)-1-(ethylsulfonyl) azetidin-3-yl)-1H-pyrazol-4-yl)-7H-pyrrolo[2, 3-d]pyrimidin-7-yl) 2-(3-phenoxyphenyl) propanoate |
| 72 (CPD-098) | 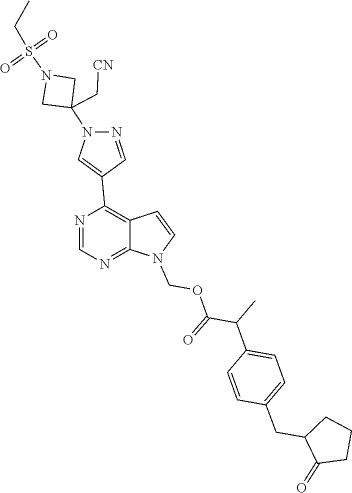 [SMILES: CCS(=O)(=O)N1CC(CC#N)(n2cc(-c3ncnc4c3ccn4COC(=O)C(C)c3ccc(CC4CCCC4=O)cc3)cn2)C1] | Methyl (4-(1-(3-(cyanomethyl)-1-(ethylsulfonyl) azetidin-3-yl)-1H-pyrazol-4-yl)-7H-pyrrolo[2, 3-d]pyrimidin-7-yl) 2-(4-((2-oxocyclopentyl) methyl)phenyl)propanoate |
| 73 (CPD-099) | 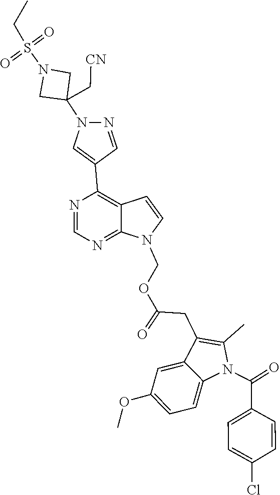 [SMILES: CCS(=O)(=O)N1CC(CC#N)(n2cc(-c3ncnc4c3ccn4COC(=O)Cc3c(C)n(C(=O)c4ccc(Cl)cc4)c4ccc(OC)cc34)cn2)C1] | Methyl (4-(1-(3-(cyanomethyl)-1-(ethylsulfonyl) azetidin-3-yl)-1H-pyrazol-4-yl)-7H-pyrrolo[2, 3-d]pyrimidin-7-yl) 2-(1-(4-chlorobenzoyl)-5-methoxy-2-methyl- 1H-indol-3-yl)acetate |
| 74 (CPD-150) | 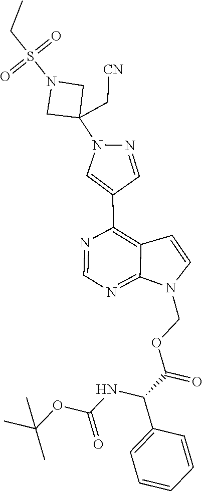 | Methyl (4-(1-(3-(cyanomethyl)-1-(ethylsulfonyl) azetidin-3-yl)-1H-pyrazol-4-yl)-7H-pyrrolo [2,3-d]pyrimidin-7-yl) (S)-2-((tert-butoxycarbonyl) amino)-2-phenylacetate |
| 75 (CPD-042) | 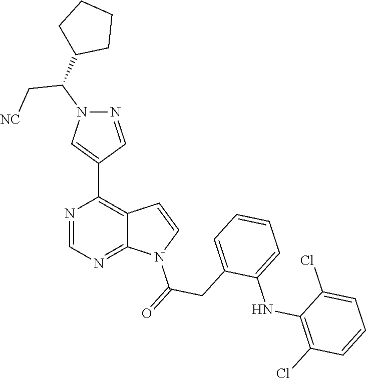 [SMILES: N#CC[C@H](C1CCCC1)n1cc(-c2ncnc3c2ccn3C(=O)Cc2ccccc2Nc2c(Cl)cccc2Cl)cn1] | (R)-3-cyclopentyl-3-(4-(7-(2-(2-((2, 6-dichlorophenyl)amino)phenyl) acetyl)-7H-pyrrolo[2,3-d]pyrimidin- 4-yl)-1H-pyrazol-1-yl)propanenitrile |
| 76 (CPD-145) | 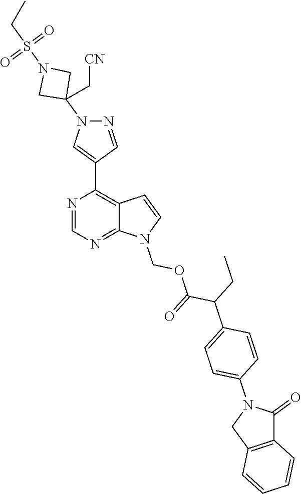 [SMILES: CCC(C(=O)OCn1ccc2c(-c3cnn(C4(CC#N)CN(S(=O)(=O)CC)C4)c3)ncnc21)c1ccc(N2Cc3ccccc3C2=O)cc1] | Methyl (4-(1-(3-(cyanomethyl)-1-(ethylsulfonyl) azetidin-3-yl)-1H-pyrazol-4-yl)-7H-pyrrolo [2,3-d]pyrimidin-7-yl) 2-(4-(1-oxoisoindolin-2-yl)phenyl)butanoate |
| 77 (CPD-044) | 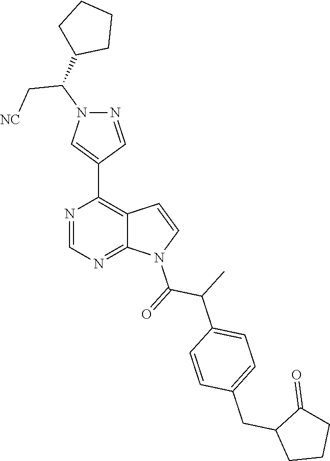 [SMILES: CC(C(=O)n1ccc2c(-c3cnn([C@H](CC#N)C4CCCC4)c3)ncnc21)c1ccc(CC2CCCC2=O)cc1] | (3R)-3-cyclopentyl-3- (4-(7-(2-(4-((2-oxocyclo- pentyl)methyl)phenyl)propanoyl)-7H-pyrrolo [2,3-d]pyrimidin-4-yl)-1H-pyrazol-1-yl) propanenitrile |
| 78 (CPD-046) | 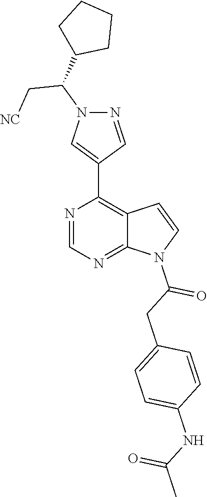 [SMILES: CC(=O)Nc1ccc(CC(=O)n2ccc3c(-c4cnn([C@H](CC#N)C5CCCC5)c4)ncnc32)cc1] | (R)-N-(4-(2-(4-(1-(2- cyano-1-cyclopentylethyl)- 1H-pyrazol-4-yl)-7H-pyrrolo[2,3-d] pyrimidin-7-yl)-2-oxoethyl)phenyl)acetamide |
| 79 (CPD-045) | 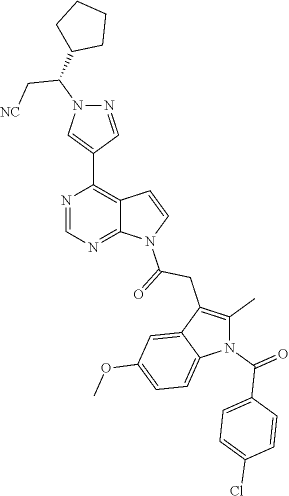 [SMILES: COc1ccc2c(c1)c(CC(=O)n1ccc3c(-c4cnn([C@H](CC#N)C5CCCC5)c4)ncnc31)c(C)n2C(=O)c1ccc(Cl)cc1] | (R)-3-(4-(7-(2-(1- (4-chlorobenzoyl)-5-methoxy- 2-methyl-1H-indol-3-yl)acetyl)-7H-pyrrolo [2,3-d]pyrimidin- 4-yl)-1H-pyrazol-1-yl)-3- cyclopentylpropanenitrile |
| 80 (CPD-049) | 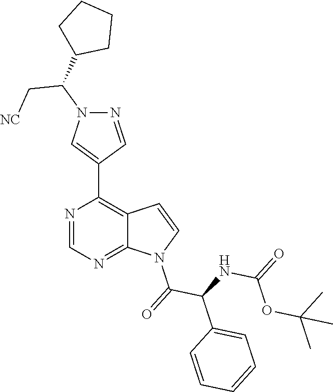 [SMILES: CC(C)(C)OC(=O)N[C@H](C(=O)n1ccc2c(-c3cnn([C@H](CC#N)C4CCCC4)c3)ncnc21)c1ccccc1] | Tert-butyl ((S)-2-(4-(1-((R)-2-cyano- 1-cyclopentylethyl)- 1H-pyrazol-4-yl)-7H- pyrrolo[2,3-d]pyrimidin- 7-yl)-2-oxo-1-phenylethyl)carbamate |
| 81 (CPD-147) | 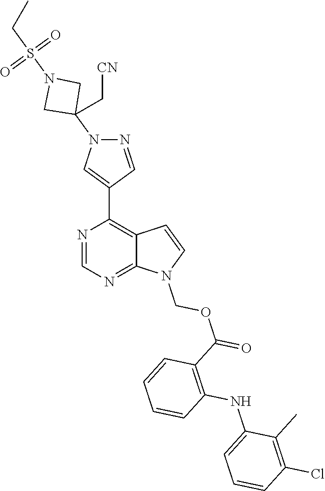 [SMILES: CCS(=O)(=O)N1CC(CC#N)(n2cc(-c3ncnc4c3ccn4COC(=O)c3ccccc3Nc3cccc(Cl)c3C)cn2)C1] | Methyl (4-(1-(3-(cyanomethyl)-1-(ethylsulfonyl) azetidin-3-yl)-1H-pyrazol-4-yl)-7H-pyrrolo[2, 3-d]pyrimidin-7-yl) 2-((3-chloro-2-methylphenyl)amino)benzoate |
| 82 (CPD-037) | 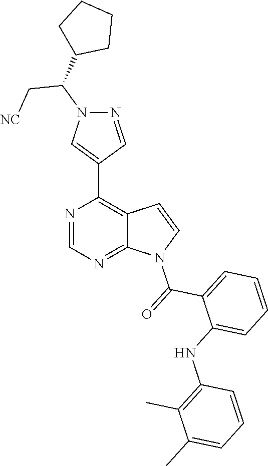 [SMILES: Cc1cccc(Nc2ccccc2C(=O)n2ccc3c(-c4cnn([C@H](CC#N)C5CCCC5)c4)ncnc32)c1C] | (R)-3-cyclopentyl-3-(4-(7-(2-((2, 3-dimethylphenyl)amino)benzoyl)-7H-pyrrolo [2,3-d]pyrimidin-4-yl)-1H-pyrazol-1-yl) propanenitrile |
| 83 (CPD-146) | 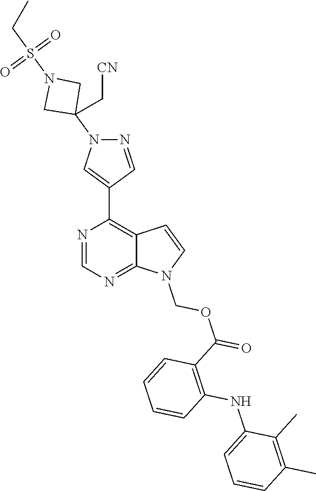 [SMILES: CCS(=O)(=O)N1CC(CC#N)(n2cc(-c3ncnc4c3ccn4COC(=O)c3ccccc3Nc3cccc(C)c3C)cn2)C1] | Methyl (4-(1-(3-(cyanomethyl)-1-(ethylsulfonyl) azetidin-3-yl)-1H-pyrazol-4-yl)-7H-pyrrolo[2, 3-d]pyrimidin-7-yl) 2-((2,3-dimethylphenyl) amino)benzoate |
| 84 (CPD-149) | 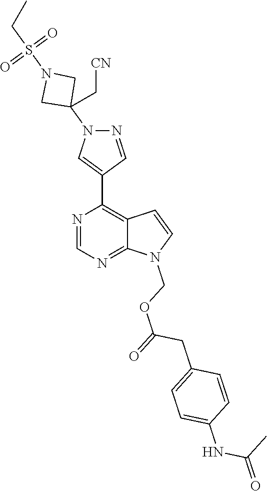 [SMILES: CCS(=O)(=O)N1CC(CC#N)(n2cc(-c3ncnc4c3ccn4COC(=O)Cc3ccc(NC(C)=O)cc3)cn2)C1] | Methyl (4-(1-(3-(cyanomethyl)-1-(ethylsulfonyl) azetidin-3-yl)-1H-pyrazol-4-yl)-7H-pyrrolo[2, 3-d]pyrimidin-7-yl) 2-(4-acetamidophenyl) acetate |
| 85 (CPD-148) | 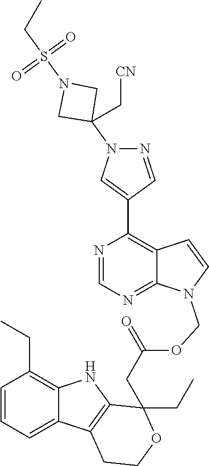 [SMILES: CCc1cccc2c3c([nH]c12)C(CC)(CC(=O)OCn1ccc2c(-c4cnn(C5(CC#N)CN(S(=O)(=O)CC)C5)c4)ncnc21)OCC3] | Methyl (4-(1-(3-(cyanomethyl)-1-(ethylsulfonyl) azetidin-3-yl)-1H-pyrazol-4-yl)-7H-pyrrolo[2, 3-d]pyrimidin-7-yl) 2-(1,8-diethyl-1,3,4, 9-tetrahydropyrano[3,4-b]indol-1-yl)acetate |
| 86 (CPD-114) | 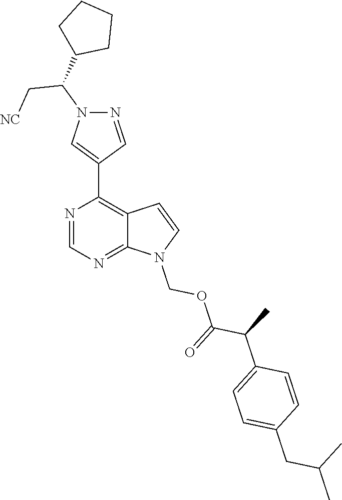 [SMILES: CC(C)Cc1ccc([C@H](C)C(=O)OCn2ccc3c(-c4cnn([C@H](CC#N)C5CCCC5)c4)ncnc32)cc1] | (4-(1-((R)-2-cyano-1-cyclopentylethyl)-1H- pyrazol-4-yl)-7H-pyrrolo [2,3-d]pyrimidin-7-yl) methyl (S)-2-(4-isobutylphenyl)propanoate |
| 87 (CPD-115) | 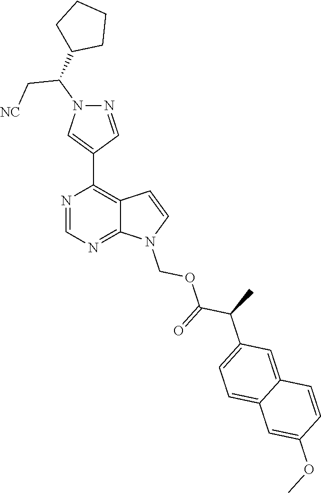 [SMILES: COc1ccc2cc([C@H](C)C(=O)OCn3ccc4c(-c5cnn([C@H](CC#N)C6CCCC6)c5)ncnc43)ccc2c1] | (4-(1-((R)-2-cyano-1-cyclopentylethyl)-1H- pyrazol-4-yl)-7H-pyrrolo [2,3-d]pyrimidin-7-yl) methyl (S)-2-(6-methoxynaphthalen-2-yl) propanoate |
| 88 (CPD-103) | 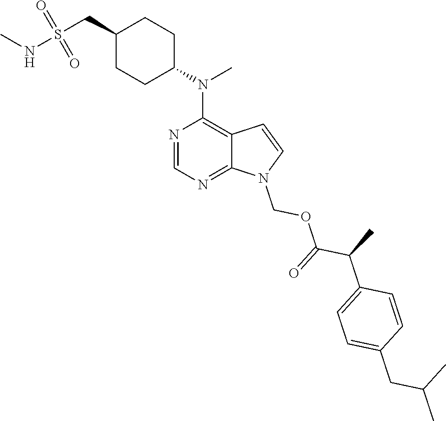 [SMILES: CNS(=O)(=O)C[C@H]1CC[C@H](N(C)c2ncnc3c2ccn3COC(=O)[C@@H](C)c2ccc(CC(C)C)cc2)CC1] | (4-(Methyl((trans)-4-((N-methylsulfamoyl) methyl)cyclohexyl)amino)-7H-pyrrolo[2, 3-d]pyrimidin-7-yl)methyl (S)-2-(4-isobutylphenyl)propanoate |
| 89 (CPD-104) | 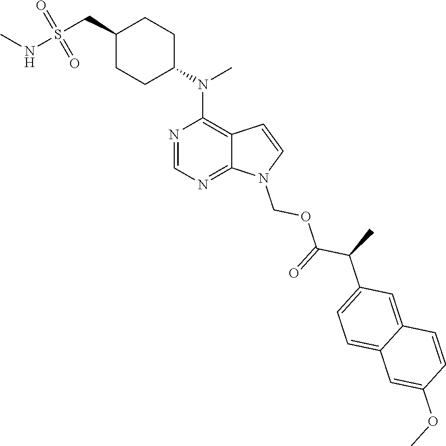 [SMILES: CNS(=O)(=O)C[C@H]1CC[C@H](N(C)c2ncnc3c2ccn3COC(=O)[C@@H](C)c2ccc3cc(OC)ccc3c2)CC1] | (4-(Methyl((trans)-4-((N-methylsulfamoyl) methyl)cyclohexyl)amino)-7H-pyrrolo[2, 3-d]pyrimidin-7-yl)methyl (S)-2-(6-methoxynaphthalen-2-yl)propanoate |
| 90 (CPD-105) | 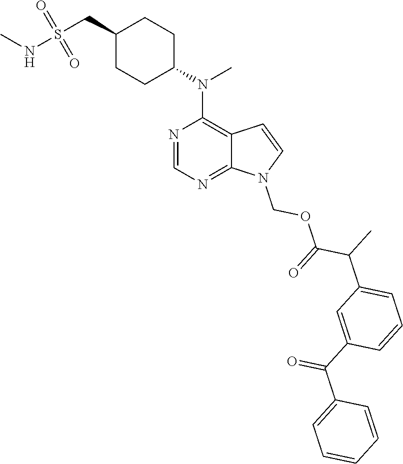 [SMILES: CNS(=O)(=O)C[C@H]1CC[C@H](N(C)c2ncnc3c2ccn3COC(=O)C(C)c2cccc(C(=O)c3ccccc3)c2)CC1] | (4-(Methyl((trans)-4-((N-methylsulfamoyl) methyl)cyclohexyl)amino)-7H-pyrrolo[2, 3-d]pyrimidin-7-yl)methyl 2-(3-benzoylphenyl)propanoate |
| 91 (CPD-162) | 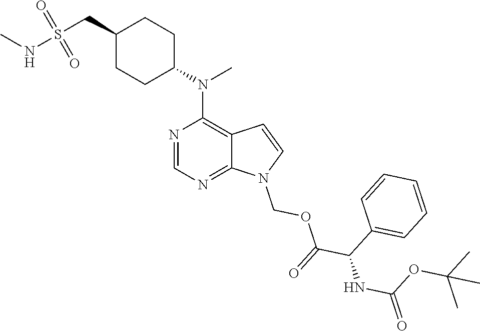 [SMILES: CNS(=O)(=O)C[C@H]1CC[C@H](N(C)c2ncnc3c2ccn3COC(=O)[C@@H](NC(=O)OC(C)(C)C)c2ccccc2)CC1] | (4-(Methyl((trans)-4-((N-methylsulfamoyl) methyl)cyclohexyl)amino)-7H-pyrrolo[2, 3-d]pyrimidin-7-yl)methyl (S)-2-((tert-butoxycarbonyl) amino)-2-phenylacetate |
| 92 (CPD-107) | 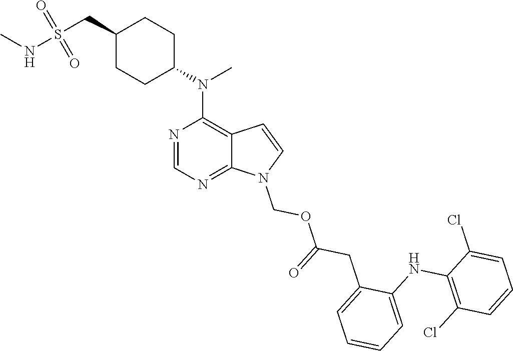 [SMILES: CNS(=O)(=O)C[C@H]1CC[C@H](N(C)c2ncnc3c2ccn3COC(=O)Cc2ccccc2Nc2c(Cl)cccc2Cl)CC1] | (4-(Methyl((trans)-4-((N-methylsulfamoyl) methyl)cyclohexyl)amino)-7H-pyrrolo[2, 3-d]pyrimidin-7-yl)methyl 2-(2-((2, 6-dichlorophenyl)amino)phenyl)acetate |
| 93 (CPD-158) | 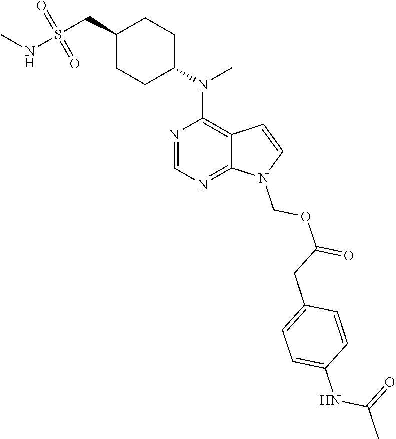 [SMILES: CNS(=O)(=O)C[C@H]1CC[C@H](N(C)c2ncnc3c2ccn3COC(=O)Cc2ccc(NC(C)=O)cc2)CC1] | (4-(Methyl((trans)-4-((N-methylsulfamoyl) methyl)cyclohexyl)amino)-7H-pyrrolo[2, 3-d]pyrimidin-7-yl)methyl 2-(4-acetamidophenyl)acetate |
| 94 (CPD-157) | 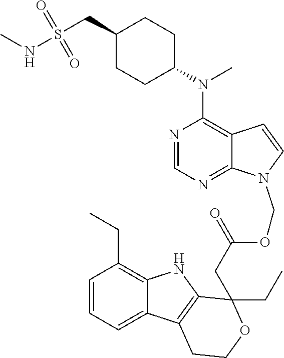 [SMILES: CCc1cccc2c3c([nH]c12)C(CC)(CC(=O)OCn1ccc2c(N(C)[C@H]4CC[C@H](CS(=O)(=O)NC)CC4)ncnc21)OCC3] | (4-(Methyl((trans)-4-((N-methylsulfamoyl) methyl)cyclohexyl)amino)-7H-pyrrolo[2, 3-d]pyrimidin-7-yl)methyl 2-(1,8-diethyl-1, 3,4,9-tetrahydropyran[3,4-b]indol-1-yl) acetate |
| 95 (CPD-033) | 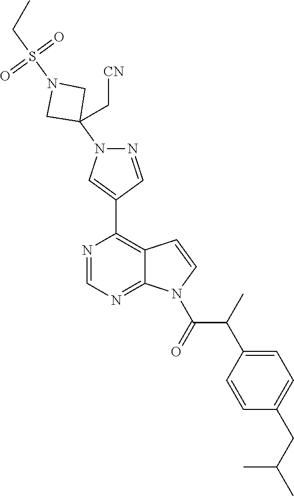 [SMILES: CCS(=O)(=O)N1CC(CC#N)(n2cc(-c3ncnc4c3ccn4C(=O)C(C)c3ccc(CC(C)C)cc3)cn2)C1] | 2-(1-(Ethylsulfonyl)-3-(4- (7-(2-(4-isobutylphenyl) propanoyl)-7H-pyrrolo[2,3-d]pyrimidin- 4-yl)-1H-pyrazol-1-yl)azetidin-3-yl) acetonitrile |
| 96 (CPD-051) | 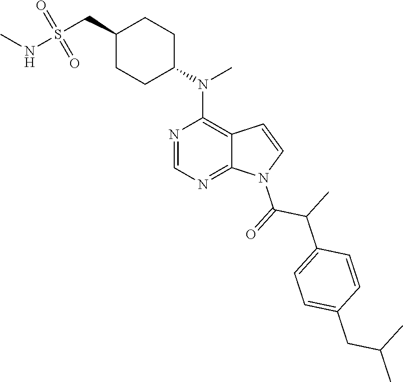 [SMILES: CNS(=O)(=O)C[C@H]1CC[C@H](N(C)c2ncnc3c2ccn3C(=O)C(C)c2ccc(CC(C)C)cc2)CC1] | 1-((Trans)-4-((7-(2-(4-isobutylphenyl) propanoyl)-7H-pyrrolo[2,3-d]pyrimidin-4-yl) (methyl)amino)cyclohexyl)-N-methyl methanesulfonamide |
| 97 (CPD-142) | 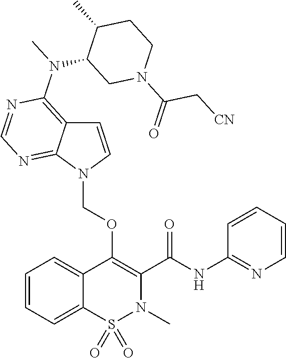 [SMILES: C[C@@H]1CCN(C(=O)CC#N)C[C@@H]1N(C)c1ncnc2c1ccn2COC1=C(C(=O)Nc2ccccn2)N(C)S(=O)(=O)c2ccccc21] | 4-((4-(((3R, 4R)-1-(2-cyanoacetyl)- 4-methylpiperidin-3-yl) (methyl)amino)-7H-pyrrolo[2,3-d]pyrimidin- 7-yl) methoxy)-2-methyl-N- (pyridin-2-yl)-2H-benzo [e][1,2]thiazine-3-carboxamide 1,1-dioxide |
| 98 (CPD-036) | 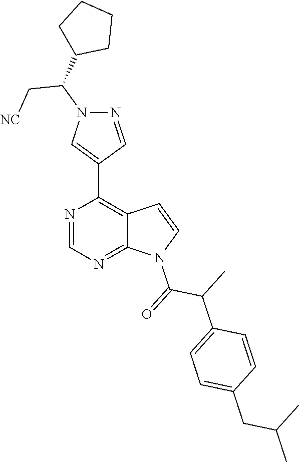 [SMILES: CC(C)Cc1ccc(C(C)C(=O)n2ccc3c(-c4cnn([C@H](CC#N)C5CCCC5)c4)ncnc32)cc1] | (R)-3-cyclopentyl-3-(4- (7-2-(4-isobutylphenyl) propanoyl)-7H-pyrrolo[2,3-d]pyrimidin- 4-yl)-1H-pyrazol-1-yl)propanenitrile |
| 99 (CPD-116) | 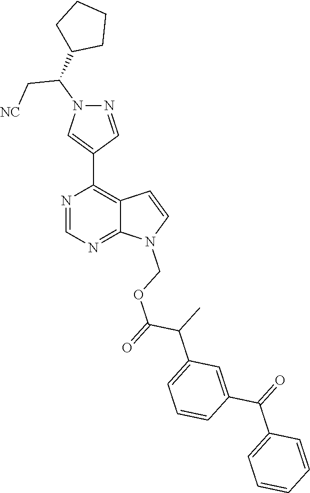 [SMILES: CC(C(=O)OCn1ccc2c(-c3cnn([C@H](CC#N)C4CCCC4)c3)ncnc21)c1cccc(C(=O)c2ccccc2)c1] | (4-(1-((R)-2-cyano-1-cyclopentylethyl)-1H- pyrazol-4-yl)-7H-pyrrolo [2,3-d]pyrimidin-7-yl) methyl 2-(3-benzoylphenyl)propanoate |
| 100 (CPD-117) | 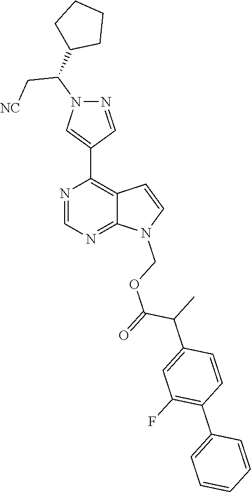 [SMILES: CC(C(=O)OCn1ccc2c(-c3cnn([C@H](CC#N)C4CCCC4)c3)ncnc21)c1ccc(-c2ccccc2)c(F)c1] | (4-(1-((R)-2-cyano-1-cyclopentylethyl)-1H- pyrazol-4-yl)-7H-pyrrolo [2,3-d]pyrimidin-7-yl) methyl 2-(2-fluoro-[1,1′-biphenyl]-4-yl) propanoate |
| 101 (CPD-118) | 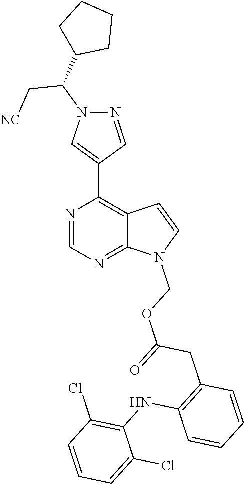 [SMILES: N#CC[C@H](C1CCCC1)n1cc(-c2ncnc3c2ccn3COC(=O)Cc2ccccc2Nc2c(Cl)cccc2Cl)cn1] | (R)-(4-(1-(2-cyano-1-cyclopentylethyl)-1H- pyrazol-4-yl)-7H-pyrrolo [2,3-d]pyrimidin-7-yl) methyl 2-(2-((2,6-dichlorophenyl)amino) phenyl)acetate |
| 102 (CPD-119) | 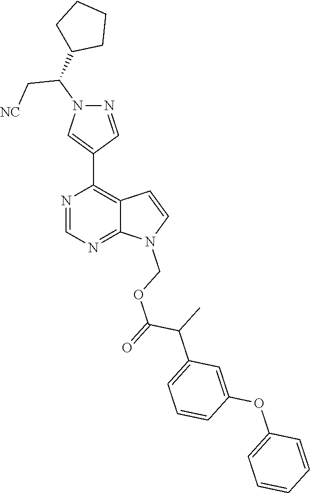 [SMILES: CC(C(=O)OCn1ccc2c(-c3cnn([C@H](CC#N)C4CCCC4)c3)ncnc21)c1cccc(Oc2ccccc2)c1] | (4-(1-((R)-2-cyano-1-cyclopentylethyl)-1H- pyrazol-4-yl)-7H-pyrrolo [2,3-d]pyrimidin-7-yl) methyl 2-(3-phenoxyphenyl)propanoate |
| 103 (CPD-120) | 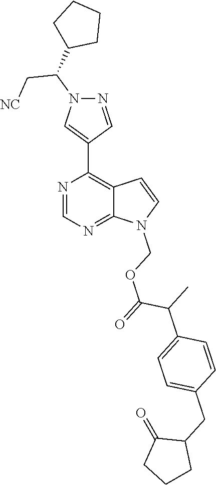 [SMILES: CC(C(=O)OCn1ccc2c(-c3cnn([C@H](CC#N)C4CCCC4)c3)ncnc21)c1ccc(CC2CCCC2=O)cc1] | (4-(1-((R)-2-cyano-1-cyclopentylethyl)-1H- pyrazol-4-yl)-7H-pyrrolo [2,3-d]pyrimidin-7-yl) methyl 2-(4-((2-oxocyclopentyl)methyl) phenyl)propanoate |
| 104 (CPD-121) | 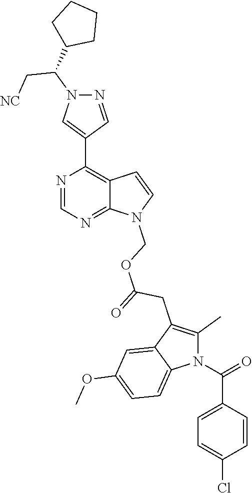 [SMILES: COc1ccc2c(c1)c(CC(=O)OCn1ccc3c(-c4cnn([C@H](CC#N)C5CCCC5)c4)ncnc31)c(C)n2C(=O)c1ccc(Cl)cc1] | (R)(4-(1-(2-cyano-1-cyclopentylethyl)-1H- pyrazol-4-yl)-7H-pyrrolo [2,3-d]pyrimidin-7-yl) methyl 2-(1-(4-chlorobenzoyl)-5-methoxy-2-methyl- 1H-indol-3-yl)acetate |
| 105 (CPD-163) | 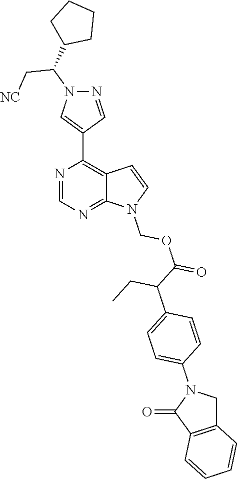 [SMILES: CCC(C(=O)OCn1ccc2c(-c3cnn([C@H](CC#N)C4CCCC4)c3)ncnc21)c1ccc(N2Cc3ccccc3C2=O)cc1] | (4-(1-((R)-2-cyano-1-cyclopentylethyl)-1H- pyrazol-4-yl)-7H-pyrrolo [2,3-d]pyrimidin-7-yl) methyl 2-(4-(1-oxoisoindolin-2-yl)phenyl) butanoate |
| 106 (CPD-164) | 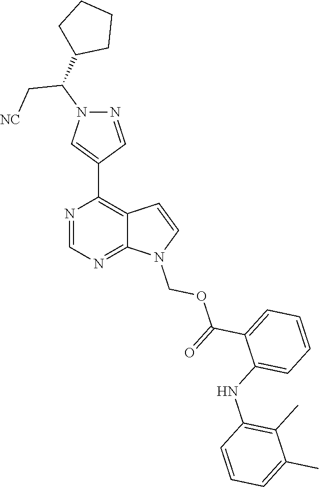 [SMILES: Cc1cccc(Nc2ccccc2C(=O)OCn2ccc3c(-c4cnn([C@H](CC#N)C5CCCC5)c4)ncnc32)c1C] | (R)-(4-(1-(2-cyano-1-cyclopentylethyl)-1H- pyrazol-4-yl)-7H-pyrrolo [2,3-d]pyrimidin-7-yl) methyl 2-((2,3-dimethylphenyl)amino) benzoate |
| 107 (CPD-165) | 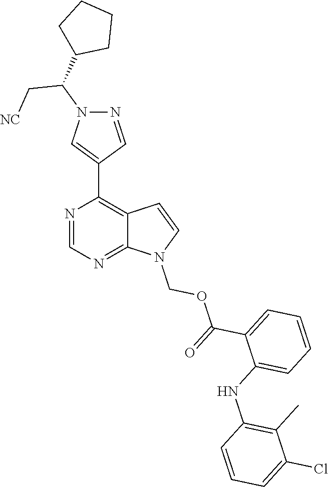 [SMILES: Cc1c(Cl)cccc1Nc1ccccc1C(=O)OCn1ccc2c(-c3cnn([C@H](CC#N)C4CCCC4)c3)ncnc21] | (R)-(4-(1-(2-cyano-1-cyclopentylethyl)-1H- pyrazol-4-yl)-7H-pyrrolo [2,3-d]pyrimidin-7-yl) methyl 2-((3-chloro-2-methylphenyl)amino) benzoate |
| 108 (CPD-184) | 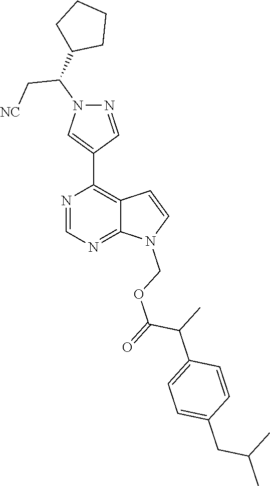 [SMILES: CC(C)Cc1ccc(C(C)C(=O)OCn2ccc3c(-c4cnn([C@H](CC#N)C5CCCC5)c4)ncnc32)cc1] | (4-(1-((R)-2-cyano-1-cyclopentylethyl)-1H- pyrazol-4-yl)-7H-pyrrolo [2,3-d]pyrimidin-7-yl) methyl 2-(4-isobutylphenyl)propanoate |
| 109 (CPD-166) | 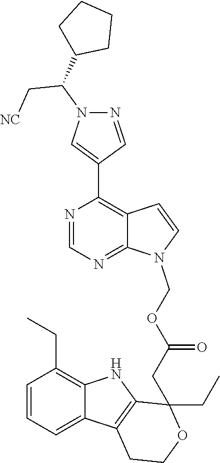 [SMILES: CCc1cccc2c3c([nH]c12)C(CC)(CC(=O)OCn1ccc2c(-c4cnn([C@H](CC#N)C5CCCC5)c4)ncnc21)OCC3] | (4-(1-((R)-2-cyano-1-cyclopentylethyl)-1H- pyrazol-4-yl)-7H-pyrrolo [2,3-d]pyrimidin-7-yl) methyl 2-(1,8-diethyl-1,3,4, 9-tetrahydropyran[3,4-b]indol-1-yl)acetate |
| 110 (CPD-167) | 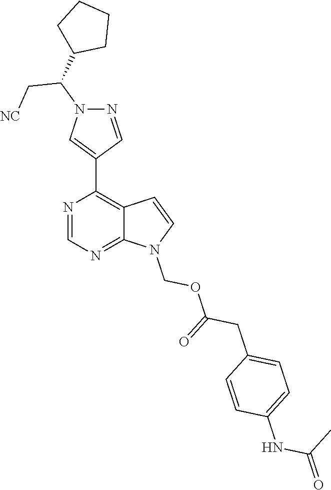 [SMILES: CC(=O)Nc1ccc(CC(=O)OCn2ccc3c(-c4cnn([C@H](CC#N)C5CCCC5)c4)ncnc32)cc1] | (R)-(4-(1-(2-cyano-1-cyclopentylethyl)-1H- pyrazol-4-yl)-7H-pyrrolo [2,3-d]pyrimidin-7-yl) methyl 2-(4-acetamidophenyl)acetate |
| 111 (CPD-168) | 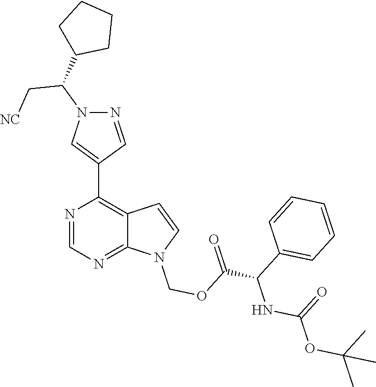 [SMILES: CC(C)(C)OC(=O)N[C@H](C(=O)OCn1ccc2c(-c3cnn([C@H](CC#N)C4CCCC4)c3)ncnc21)c1ccccc1] | (4-(1-((R)-2-cyano-1-cyclopentylethyl)-1H- pyrazol-4-yl)-7H-pyrrolo [2,3-d]pyrimidin-7-yl) methyl (S)-2-((tert-butoxycarbonyl) amino)-2-phenylacetate |
| 112 (CPD-182) | 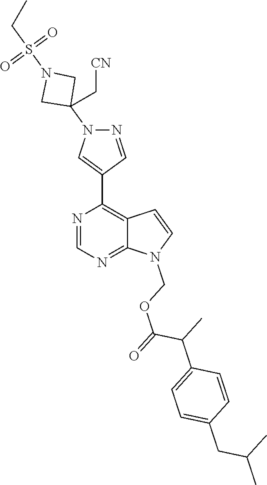 [SMILES: CCS(=O)(=O)N1CC(CC#N)(n2cc(-c3ncnc4c3ccn4COC(=O)C(C)c3ccc(CC(C)C)cc3)cn2)C1] | Methyl (4-(1-(3-(cyanomethyl)-1-(ethylsulfonyl) azetidin-3-yl)-1H-pyrazol-4-yl)-7H-pyrrolo [2,3-d]pyrimidin-7-yl) 2-(4-isobutylphenyl) propanoate |
| 113 (CPD-106) | 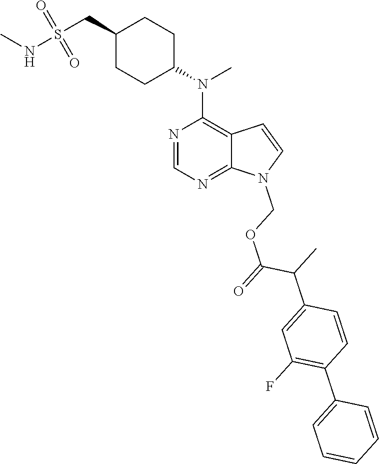 | (4-(Methyl((trans)-4-((N-methylsulfamoyl) methyl)cyclohexyl)amino)-7H-pyrrolo[2, 3-d]pyrimidin-7-yl)methyl 2-(2-fluoro-[1, 1′-biphenyl]-4-yl) propanoate |
| 114 (CPD-108) | 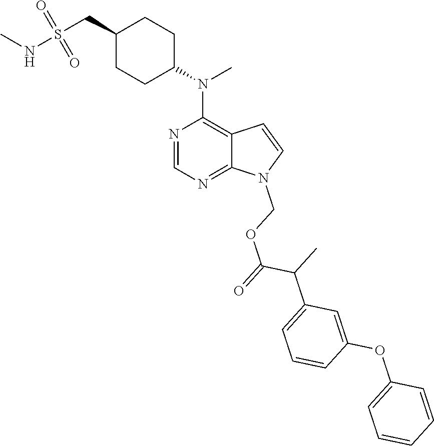 | (4-(Methyl((trans)-4-((N-methylsulfamoyl) methyl)cyclohexyl)amino)-7H-pyrrolo[2, 3-d]pyrimidin-7-yl)methyl 2-(3-phenoxyphenyl)propanoate |
| 115 (CPD-109) | 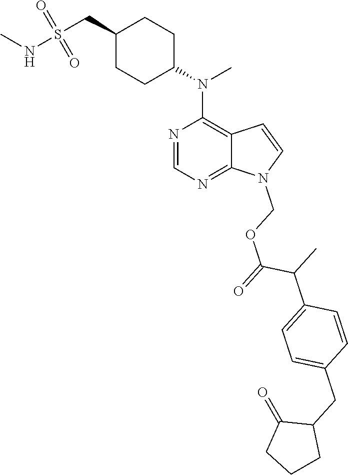 | (4-(Methyl((trans)-4-((N-methylsulfamoyl) methyl)cyclohexyl)amino)-7H-pyrrolo[2, 3-d]pyrimidin-7-yl)methyl 2-(4-((2-oxocyclopentyl)methyl)phenyl) propanoate |
| 116 (CPD-110) | 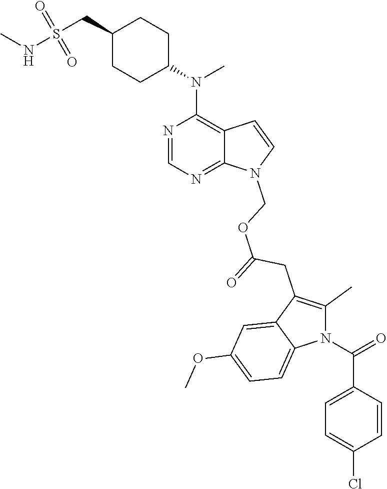 | (4-(Methyl((trans)-4-((N-methylsulfamoyl) methyl)cyclohexyl)amino)-7H-pyrrolo[2, 3-d]pyrimidin-7-yl)methyl 2-(1-(4-chlorobenzoyl)-5-methoxy-2-methyl- 1H-indol-3-yl)acetate |
| 117 (CPD-154) | 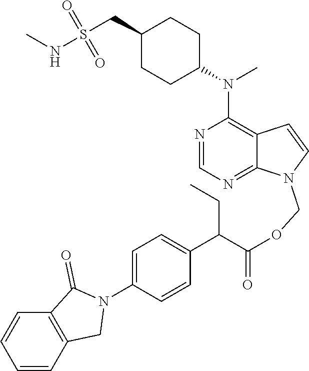 | (4-(Methyl((trans)-4-((N-methylsulfamoyl) methyl)cyclohexyl)amino)-7H-pyrrolo[2, 3-d]pyrimidin-7-yl)methyl 2-(4-(1-) oxoisoindolin-2-yl)phenyl)butanoate |
| 118 (CPD-155) | 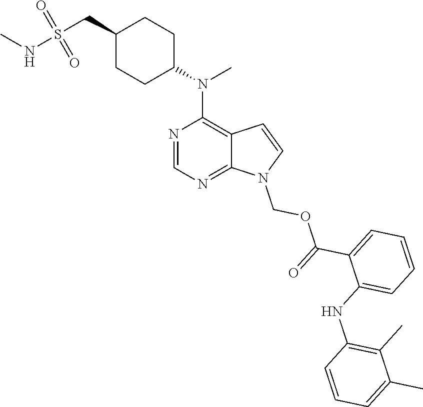 | (4-(Methyl((trans)-4-((N-methylsulfamoyl) methyl)cyclohexyl)amino)-7H-pyrrolo[2, 3-d]pyrimidin-7-yl)methyl 2-((2, 3-dimethylphenyl)amino)benzoate |
| 119 (CPD-183) | 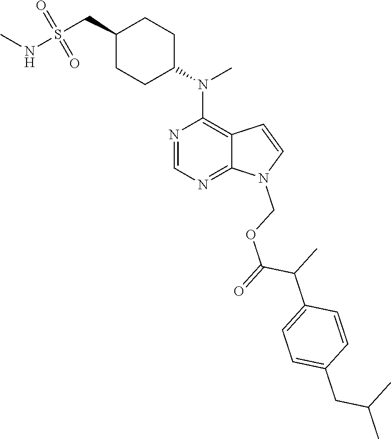 | (4-(Methyl((trans)-4-((N-methylsulfamoyl) methyl)cyclohexyl)amino)-7H-pyrrolo[2, 3-d]pyrimidin-7-yl)methyl 2-(4-isobutylphenyl)propanoate |
| 120 (CPD-156) | 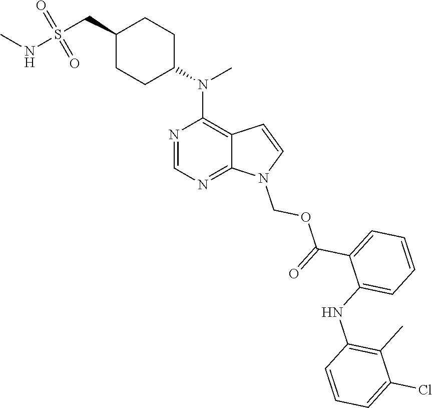 | (4-(Methyl((trans)-4-((N-methylsulfamoyl) methyl)cyclohexyl)amino)-7H-pyrrolo[2, 3-d]pyrimidin-7-yl)methyl 2-((3-chloro-2-methylphenyl)amino)benzoate |
| 121 (CPD-151) | 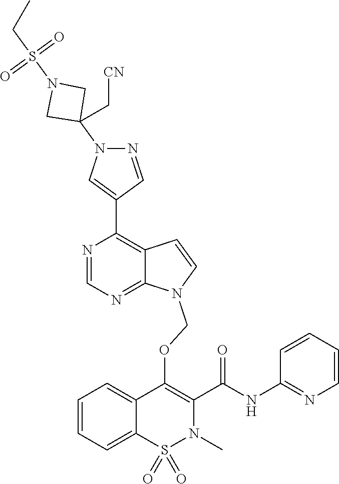 [SMILES: CCS(=O)(=O)N1CC(CC#N)(n2cc(-c3ncnc4c3ccn4COC3=C(C(=O)Nc4ccccn4)N(C)S(=O)(=O)c4ccccc43)cn2)C1] | 4-((4-(1-(3-(Cyanomethyl)-1-(ethylsulfonyl) azetidin-3-yl)-1H-pyrazol-4-yl)-7H-pyrrolo [2,3-d]pyrimidin-7-yl) methoxy)-2-methyl-N- (pyridin-2-yl)-2H-benzo [e][1,2]thiazine-3-carboxamide 1,1-dioxide |
| 122 (CPD-101) | 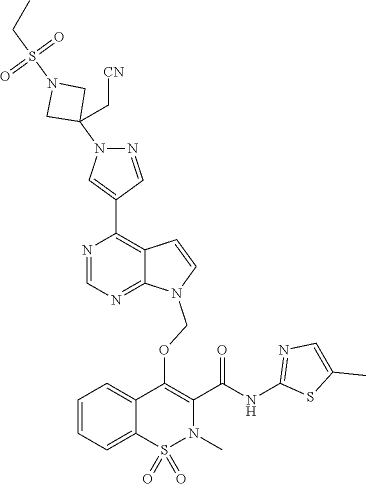 [SMILES: CCS(=O)(=O)N1CC(CC#N)(n2cc(-c3ncnc4c3ccn4COC3=C(C(=O)Nc4ncc(C)s4)N(C)S(=O)(=O)c4ccccc43)cn2)C1] | 4-((4-(1-(3-(Cyanomethyl)-1-(ethylsulfonyl) azetidin-3-yl)-1H-pyrazol-4-yl)-7H-pyrrolo [2,3-d]pyrimidin-7-yl) methoxy)-2-methyl-N-(5-methylthiazol-2-yl)- 2H-benzo[e][1,2]thiazine-3-carboxamide 1, 1-dioxide |
| 123 (CPD-066) | 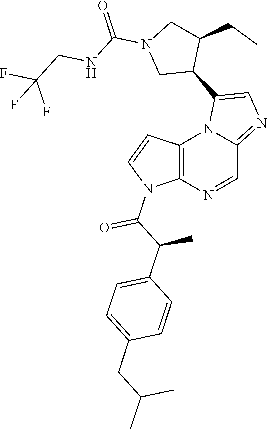 [SMILES: CC[C@@H]1CN(C(=O)NCC(F)(F)F)C[C@@H]1c1cnc2cnc3c(ccn3C(=O)[C@@H](C)c3ccc(CC(C)C)cc3)n12] | (3S,4R)-3-ethyl-4-(3-((S)-2-(4-isobutylphenyl) propanoyl)-3H-imidazo[1,2-a]pyrrolo[2,3-e] pyrazin-8-yl)-N-(2,2,2-trifluoroethyl) pyrrolidine-1-carboxamide |
| 124 (CPD-067) | 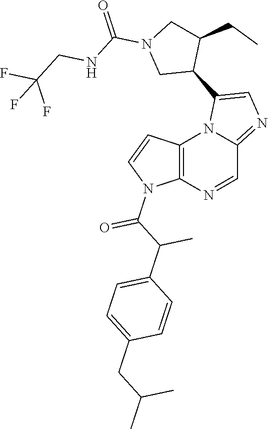 [SMILES: CC[C@@H]1CN(C(=O)NCC(F)(F)F)C[C@@H]1c1cnc2cnc3c(ccn3C(=O)C(C)c3ccc(CC(C)C)cc3)n12] | (3S,4R)-3-ethyl-4-(3- ((R)-2-(4-isobutylphenyl) propanoyl)-3H-imidazo[1,2-a]pyrrolo[2,3-e] pyrazin-8-yl)-N-(2,2,2-trifluoroethyl) pyrrolidine-1-carboxamide |
| 125 (CPD-072) | 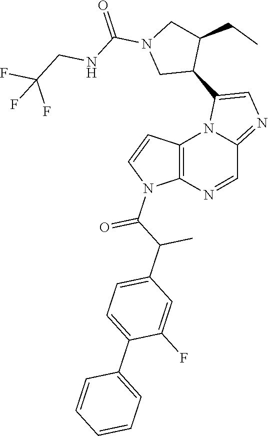 [SMILES: CC[C@@H]1CN(C(=O)NCC(F)(F)F)C[C@@H]1c1cnc2cnc3c(ccn3C(=O)C(C)c3ccc(-c4ccccc4)c(F)c3)n12] | (3S,4R)-3-ethyl-4-(3-(2-(2-fluoro-[1, 1′-biphenyl]-4-yl)propanoyl)-3H-imidazo[1, 2-a]pyrrolo[2,3-e]pyrazin-8-yl)-N-(2,2, 2-trifluoroethyl)pyrrolidine-1-carboxamide |
| 126 (CPD-071) | 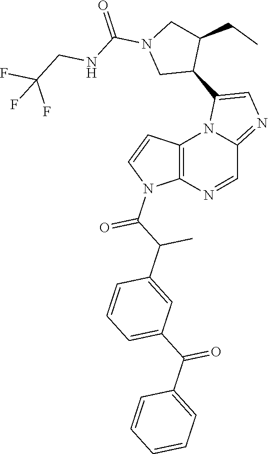 [SMILES: CC[C@@H]1CN(C(=O)NCC(F)(F)F)C[C@@H]1c1cnc2cnc3c(ccn3C(=O)C(C)c3cccc(C(=O)c4ccccc4)c3)n12] | (3R,4S)-3-(3-(2-(3-benzoylphenyl) propanoyl)-3H-imidazo[1,2-a]pyrrolo[2,3-e] pyrazin-8-yl)-4-ethyl-N-(2,2,2-trifluoroethyl) pyrrolidine-1-carboxamide |
| 127 (CPD-073) | 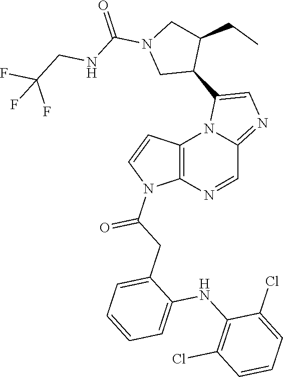 [SMILES: CC[C@@H]1CN(C(=O)NCC(F)(F)F)C[C@@H]1c1cnc2cnc3c(ccn3C(=O)Cc3ccccc3Nc3c(Cl)cccc3Cl)n12] | (3R,4S)-3-(3-(2-(2-((2,6-dichlorophenyl) amino)phenyl)acetyl)-3H-imidazo[1,2-a] pyrrolo[2,3-e]pyrazin-8-yl)-4-ethyl-N-(2,2, 2-trifluoroethyl)pyrrolidine-1-carboxamide |
| 128 (CPD-074) | 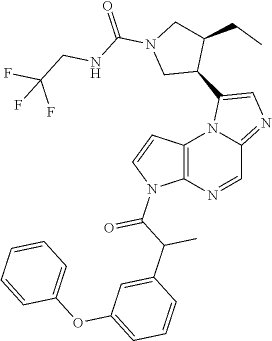 [SMILES: CC[C@@H]1CN(C(=O)NCC(F)(F)F)C[C@@H]1c1cnc2cnc3c(ccn3C(=O)C(C)c3cccc(Oc4ccccc4)c3)n12] | (3S,4R)-3-ethyl-4-(3-(2-(3-phenoxyphenyl) propanoyl)-3H-imidazo[1,2-a]pyrrolo[2,3-e] pyrazin-8-yl)-N-(2,2,2-trifluoroethyl) pyrrolidine-1-carboxamide |
| 129 (CPD-068) | 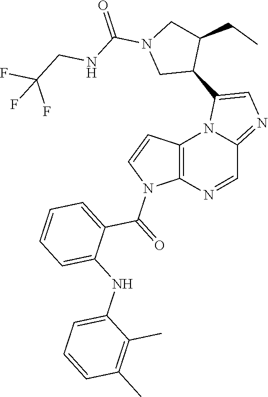 [SMILES: CC[C@@H]1CN(C(=O)NCC(F)(F)F)C[C@@H]1c1cnc2cnc3c(ccn3C(=O)c3ccccc3Nc3cccc(C)c3C)n12] | (3R,4S)-3-(3-(2-((2,3-dimethylphenyl)amino) benzoyl)-3H-imidazo[1,2-a]pyrrolo[2,3-e] pyrazin-8-yl)-4-ethyl-N- (2,2,2-trifluoroethyl) pyrrolidine-1-carboxamide |
| 130 (CPD-069) | 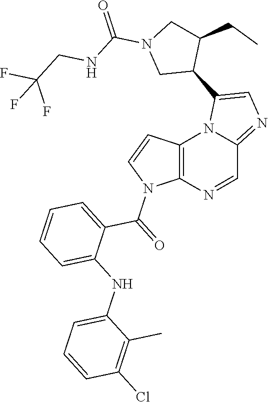 [SMILES: CC[C@@H]1CN(C(=O)NCC(F)(F)F)C[C@@H]1c1cnc2cnc3c(ccn3C(=O)c3ccccc3Nc3cccc(Cl)c3C)n12] | (3R,4S)-3-(3-(2-((3-chloro-2-methylphenyl) amino)benzoyl)-3H-imidazo[1,2-a]pyrrolo [2,3-e]pyrazin-8-yl)-4-ethyl-N-(2,2, 2-trifluoroethyl)pyrrolidine-1-carboxamide |
| 131 (CPD-070) | 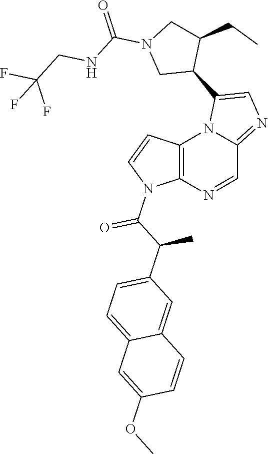 [SMILES: CC[C@@H]1CN(C(=O)NCC(F)(F)F)C[C@@H]1c1cnc2cnc3c(ccn3C(=O)[C@@H](C)c3ccc4cc(OC)ccc4c3)n12] | (3S,4R)-3-ethyl-4-(3-((S)-2-(6-) methoxynaphthalen-2-yl) propanoyl)-3H-imidazo[1,2-a]pyrrolo[2,3-e] pyrazin-8-yl)-N-(2,2,2-trifluoroethyl) pyrrolidine-1-carboxamide |
| 132 (CPD-075) | 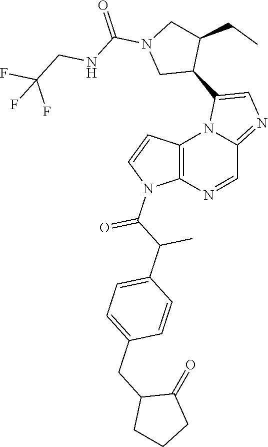 [SMILES: CC[C@@H]1CN(C(=O)NCC(F)(F)F)C[C@@H]1c1cnc2cnc3c(ccn3C(=O)C(C)c3ccc(CC4CCCC4=O)cc3)n12] | (3S, 4R)-3-ethyl-4-(3-(2-(4-((2-oxocyclopentyl) methyl)phenyl)propanoyl)-3H-imidazo[1, 2-a]pyrrolo[2,3-e]pyrazin-8-yl)-N-(2,2, 2-trifluoroethyl)pyrrolidine-1-carboxamide |
| 133 (CPD-076) | 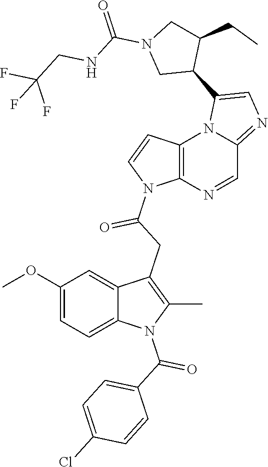 [SMILES: CC[C@@H]1CN(C(=O)NCC(F)(F)F)C[C@@H]1c1cnc2cnc3c(ccn3C(=O)Cc3c(C)n(C(=O)c4ccc(Cl)cc4)c4ccc(OC)cc34)n12] | (3R,4S)-3-(3-(2-(1- (4-Chlorobenzoyl)-5-methoxy-2- methyl-1H-indol-3-yl)acetyl)-3H-imidazo[1, 2-a]pyrrolo[2,3-e]pyrazin- 8-yl)-4-ethyl-N-(2,2,2-trifluoroethyl) pyrrolidine-1-carboxamide |
| 134 (CPD-077) | 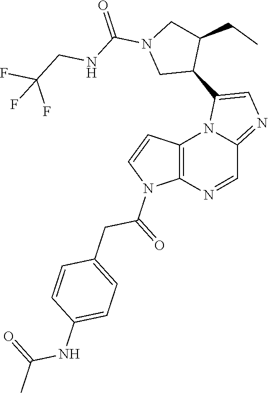 [SMILES: CC[C@@H]1CN(C(=O)NCC(F)(F)F)C[C@@H]1c1cnc2cnc3c(ccn3C(=O)Cc3ccc(NC(C)=O)cc3)n12] | (3R,4S)-3-(3-(2-(4-acetamidophenyl) acetyl)-3H-imidazo[1,2-a]pyrrolo[2,3-e] pyrazin-8-yl)-4-ethyl-N-(2,2,2-trifluoroethyl) pyrrolidine-1-carboxamide |
| 135 (CPD-078) | 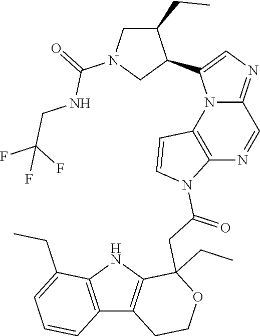 [SMILES: CCc1cccc2c3c([nH]c12)C(CC)(CC(=O)n1ccc2c1ncc1ncc([C@H]4CN(C(=O)NCC(F)(F)F)C[C@H]4CC)n12)OCC3] | (3R,4S)-3-(3-(2-(1,8-diethyl-1,3,4, 9-tetrahydropyrano[3,4-b]indol-1-yl) acetyl)-3H-imidazo[1,2-a]pyrrolo[2,3-e] pyrazin-8-yl)-4-ethyl-N-(2,2,2-trifluoroethyl) pyrrolidine-1-carboxamide |
| 136 (CPD-079) | 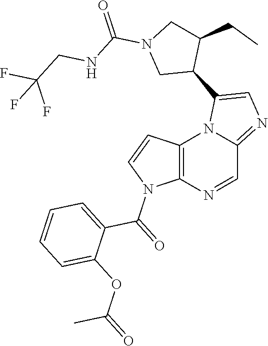 [SMILES: CC[C@@H]1CN(C(=O)NCC(F)(F)F)C[C@@H]1c1cnc2cnc3c(ccn3C(=O)c3ccccc3OC(C)=O)n12] | 2-(8-((3R,4S)-4-ethyl-1-((2,2, 2-trifluoroethyl)carbamoyl)pyrrolidin- 3-yl)-3H-imidazo[1,2-a]pyrrolo[2,3-e] pyrazine-3-carbonyl)phenylacetate |
| 137 (CPD-080) | 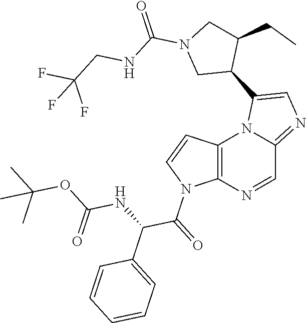 [SMILES: CC[C@@H]1CN(C(=O)NCC(F)(F)F)C[C@@H]1c1cnc2cnc3c(ccn3C(=O)[C@@H](NC(=O)OC(C)(C)C)c3ccccc3)n12] | Tert-butyl ((S)-2-(8-((3R,4S)-4-ethyl-1-((2,2, 2-trifluoroethyl)carbamoyl)pyrrolidin- 3-yl)-3H-imidazo[1,2-a]pyrrolo[2,3-e] pyrazin-3-yl)-2-oxo-1-phenethyl)carbamate |
| 138 (CPD-081) | 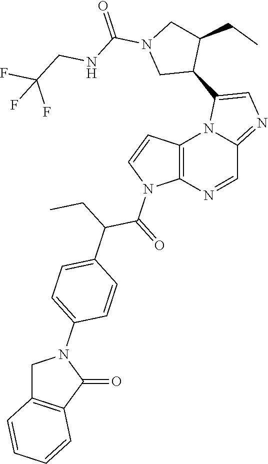 [SMILES: CCC(C(=O)n1ccc2c1ncc1ncc([C@H]3CN(C(=O)NCC(F)(F)F)C[C@H]3CC)n12)c1ccc(N2Cc3ccccc3C2=O)cc1] | (3S,4R)-3-ethyl-4-(3-(2-(4-(1-)oxoisoindolin- 2-yl)phenyl)butanoyl)-3H-imidazo[1,2-a] pyrrolo[2,3-e]pyrazin-8-yl)-N-(2,2, 2-trifluoroethyl)pyrrolidine-1-carboxamide |
| 139 (CPD-186) | 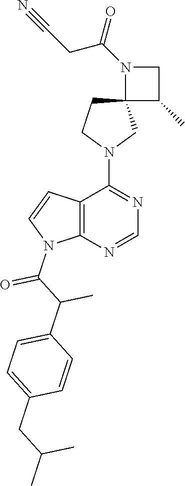 [SMILES: CC(C)Cc1ccc(C(C)C(=O)n2ccc3c(N4CC[C@]5(C4)[C@@H](C)CN5C(=O)CC#N)ncnc32)cc1] | 3-((3S,4R)-6-(7-(2-(4-isobutylphenyl) propanoyl)-7H-pyrrolo[2,3-d]pyrimidin- 4-yl)-3-methyl-1,6-diazaspiro[3.4]oct- 1-yl)-3-oxopropanenitrile |
| 140 (CPD-187) | 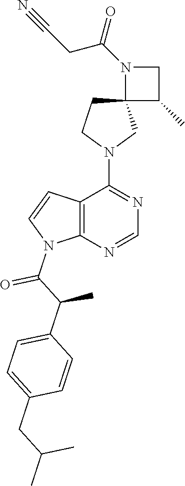 [SMILES: CC(C)Cc1ccc([C@H](C)C(=O)n2ccc3c(N4CC[C@]5(C4)[C@@H](C)CN5C(=O)CC#N)ncnc32)cc1] | 3-((3S,4R)-6-(7-((S)-2-(4-isobutylphenyl) propanoyl)-7H-pyrrolo[2,3-d]pyrimidin- 4-yl)-3-methyl-1,6-diazaspiro[3.4]oct- 1-yl)-3-oxopropanenitrile |
| 141 (CPD-190) | 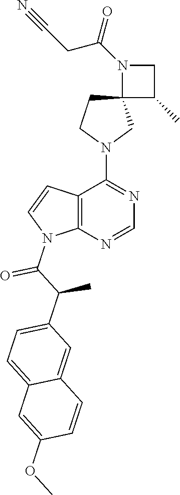 [SMILES: COc1ccc2cc([C@H](C)C(=O)n3ccc4c(N5CC[C@]6(C5)[C@@H](C)CN6C(=O)CC#N)ncnc43)ccc2c1] | 3-((3S,4R)- 6-(7-((S)-2-(6-methoxynaphthalen-2-yl) propanoyl)-7H-pyrrolo[2,3-d]pyrimidin- 4-yl)-3-methyl-1,6-diazaspiro[3.4]octan- 1-yl)-3-oxopropanenitrile |
| 142 (CPD-090) | 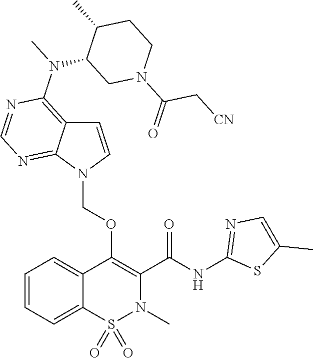 [SMILES: Cc1cnc(NC(=O)C2=C(OCn3ccc4c(N(C)[C@H]5CN(C(=O)CC#N)CC[C@H]5C)ncnc43)c3ccccc3S(=O)(=O)N2C)s1] | 4-((4-(((3R, 4R)-1-(2-cyanoacetyl)- 4-methylpiperidin-3-yl) (methyl)amino)-7H-pyrrolo[2,3-d] pyrimidin-7-yl) methoxy)-2-methyl-N- (5-methylthiazol-2-yl)- 2H-benzo[e][1,2]thiazine-3-carboxamide 1,1-dioxide |
| 143 (CPD-201) | 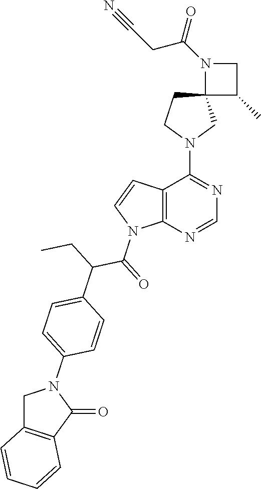 [SMILES: CCC(C(=O)n1ccc2c(N3CC[C@]4(C3)[C@@H](C)CN4C(=O)CC#N)ncnc21)c1ccc(N2Cc3ccccc3C2=O)cc1] | 3-((3S, 4R)-3-methyl-6-(7-(2- (4-(1-oxoisoindolin-2-yl) phenyl)butanoyl)-7H-pyrrolo[2,3-d] pyrimidin-4-yl)-1,6-diazaspiro[3.4]octan- 1-yl)-3-oxopropanenitrile |
| 144 (CPD-202) | 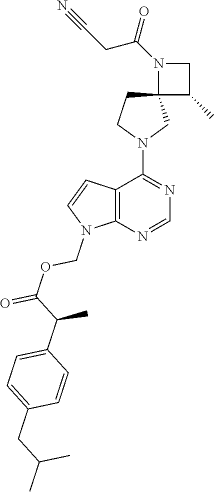 [SMILES: CC(C)Cc1ccc([C@H](C)C(=O)OCn2ccc3c(N4CC[C@]5(C4)[C@@H](C)CN5C(=O)CC#N)ncnc32)cc1] | (4-((3S,4R)-1-(2-cyanoacetyl)-3-methyl-1, 6-diazaspiro[3.4]octan-6-yl)-7H-pyrrolo[2, 3-d]pyrimidin-7-yl)methyl (S)-2-(4-isobutylphenyl)propanoate |
| 145 (CPD-191) | 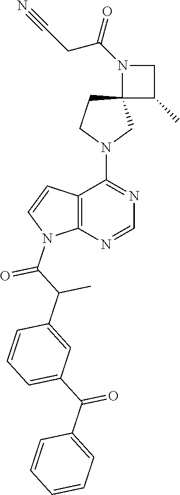 [SMILES: CC(C(=O)n1ccc2c(N3CC[C@]4(C3)[C@@H](C)CN4C(=O)CC#N)ncnc21)c1cccc(C(=O)c2ccccc2)c1] | 3-((3S,4R)-6-(7-(2-(3-benzoylphenyl) propanoyl)-7H-pyrrolo[2,3-d]pyrimidin- 4-yl)-3-methyl-1,6-diazaspiro[3.4]oct- 1-yl)-3-oxopropanenitrile |
| 146 (CPD-143) | 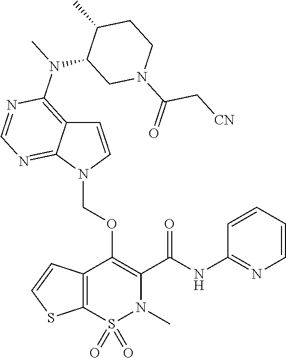 [SMILES: C[C@@H]1CCN(C(=O)CC#N)C[C@@H]1N(C)c1ncnc2c1ccn2COC1=C(C(=O)Nc2ccccn2)N(C)S(=O)(=O)c2sccc21] | 4-((4-(((3R, 4R)-1-(2-cyanoacetyl)- 4-methylpiperidin-3-yl) (methyl)amino)-7H-pyrrolo[2,3-d] pyrimidin-7-yl) methoxy)-2-methyl-N- (pyridin-2-yl)-2H-thieno [3,2-e][1,2]thiazine-3-carboxamide 1,1-dioxide |
| 147 (CPD-159) | 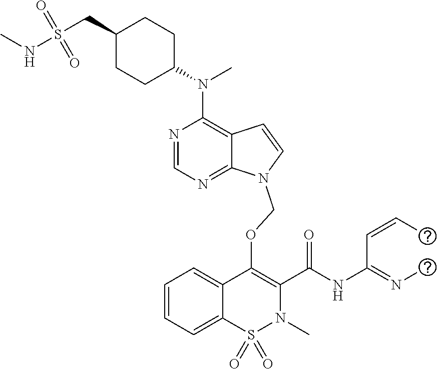 | 2-Methyl-4-((4-(methyl ((trans)-4-((N-methylsulfamoyl)methyl) cyclohexyl)amino)-7H-pyrrolo[2,3-d] pyrimidin-7-yl) methoxy)-N-(pyridin-2-yl)-2H-benzo[e][1,2] thiazine-3-carboxamide 1,1-dioxide |
| 148 (CPD-152) | 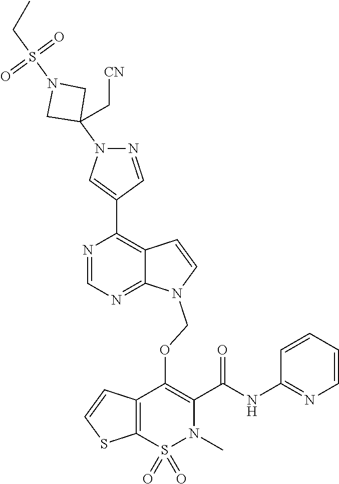 [SMILES: CCS(=O)(=O)N1CC(CC#N)(n2cc(-c3ncnc4c3ccn4COC3=C(C(=O)Nc4ccccn4)N(C)S(=O)(=O)c4sccc43)cn2)C1] | 4-((4-(1-(3-(Cyanomethyl)-1-(ethylsulfonyl) azetidin-3-yl)-1H-pyrazol-4-yl)-7H-pyrrolo [2,3-d]pyrimidin-7-yl) methoxy)-2-methyl-N- (pyridin-2-yl)-2H-thieno [3,2-e][1,2]thiazine-3-carboxamide 1,1-dioxide |
| 149 (CPD-160) | 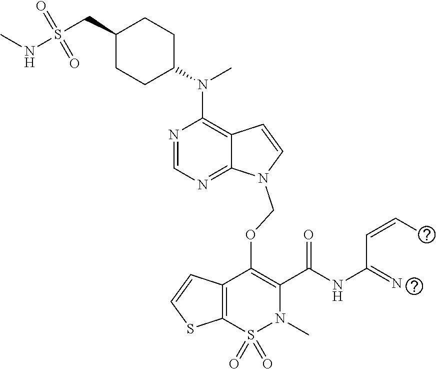 | 2-Methyl-4-((4-(methyl ((trans)-4-((N-methylsulfamoyl)methyl) cyclohexyl)amino)-7H-pyrrolo[2,3-d] pyrimidin-7-yl) methoxy)-N-(pyridin-2-yl)-2H-thieno[3,2-e] [1,2]thiazine-3-carboxamide 1,1-dioxide |

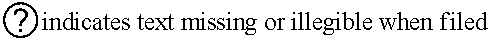

### Example 1

### 3-((3R, 4R)-3-((7-(2-(4-isobutylphenyl) propanoyl)-7H-pyrrolo [2, 3-d] pyrimidin-4-yl) (methyl) amino)-4-methylpiperidin-1-yl)-3-oxopropanenitrile

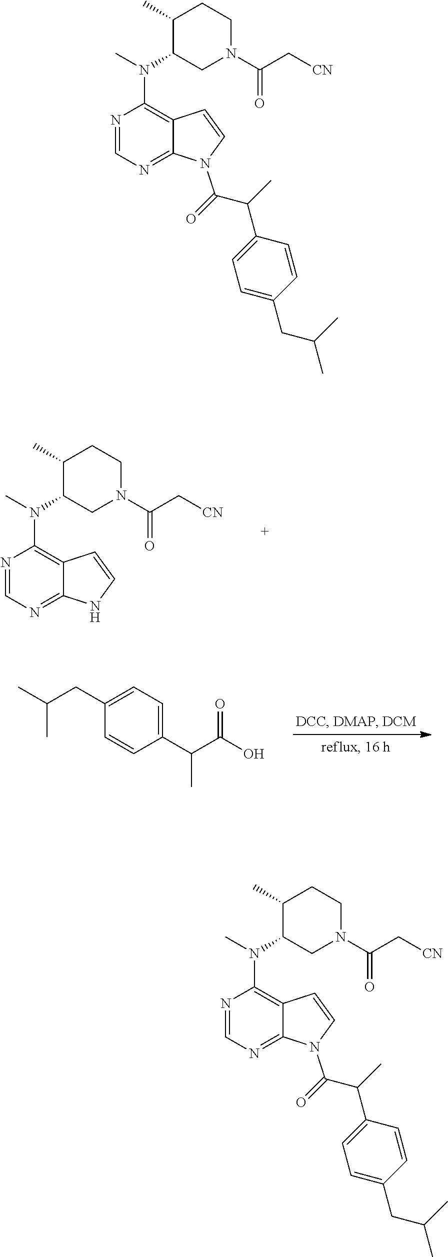 [SMILES: CC(C)Cc1ccc(C(C)C(=O)n2ccc3c(N(C)[C@H]4CN(C(=O)CC#N)CC[C@H]4C)ncnc32)cc1 ; C[C@@H]1CCN(C(=O)CC#N)C[C@@H]1N(C)c1ncnc2[nH]ccc12 ; "+" ; CC(C)Cc1ccc(C(C)C(=O)O)cc1 ; CC(C)Cc1ccc(C(C)C(=O)n2ccc3c(N(C)[C@H]4CN(C(=O)CC#N)CC[C@H]4C)ncnc32)cc1]

### Synthesis of 3-((3R, 4R)-3-((7-(2-(4-isobutylphenyl) propanoyl)-7H-pyrrolo [2, 3-d] pyrimidin-4-yl) (methyl) amino)-4-methylpiperidin-1-yl)-3-oxopropanenitrile

3-{(3R, 4R)-4-methyl-3-[methyl-(7H-pyrrolo [2, 3-d] pyrimidin-4-yl)-piperidin-1-yl]-3-oxo-propionitrile (tofacitinib, 312 mg, 1 mmol) and 2-(4-isobutylphenyl) propanoic acid (ibuprofen, 412 mg, 2 mmol) were dissolved in dichloromethane (20 mL). 4-Dimethylaminopyridine (DMAP, 134 mg, 1.1 mmol) and dicyclohexyl carbodiimide (DCC, 412 mg, 2 mmol) were added in an ice water bath, followed by stirring at reflux for 16 hours. After completion of the reaction, the reaction solution was filtered. The filtrate was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 2:1) to give the title compound as a white solid, 0.292 g, 58% yield. MS (m/z): [M+H]⁺ calcd for C₂₉H₃₆N₆O₂, 501.65; found, 501.2.1H NMR (400 MHz, Chloroform-d) δ 8.39 (d, J=7.3 Hz, 1H), 7.68 (dd, J=15.2, 4.2 Hz, 1H), 7.44-7.33 (m, 2H), 7.08-7.03 (m, 2H), 6.61 (t, J=4.2 Hz, 1H), 6.13 (tt, J=7.3, 3.5 Hz, 1H), 5.09 (s, 1H), 4.09-3.70 (m, 2H), 3.66-3.44 (m, 4H), 3.33 (d, J=16.4 Hz, 3H), 2.39 (d, J=7.2 Hz, 3H), 1.88-1.70 (m, 2H), 1.67-1.60 (m, 4H), 1.15-1.03 (m, 3H), 0.86 (d, J=6.6 Hz, 6H).

### Example 2

### 3-((3R, 4R)-3-((7-((S)-2-(4-isobutylphenyl) propanoyl)-7H-pyrrolo [2, 3-d] pyrimidin-4-yl) (methyl) amino)-4-methylpiperidin-1-yl)-3-oxopropanenitrile

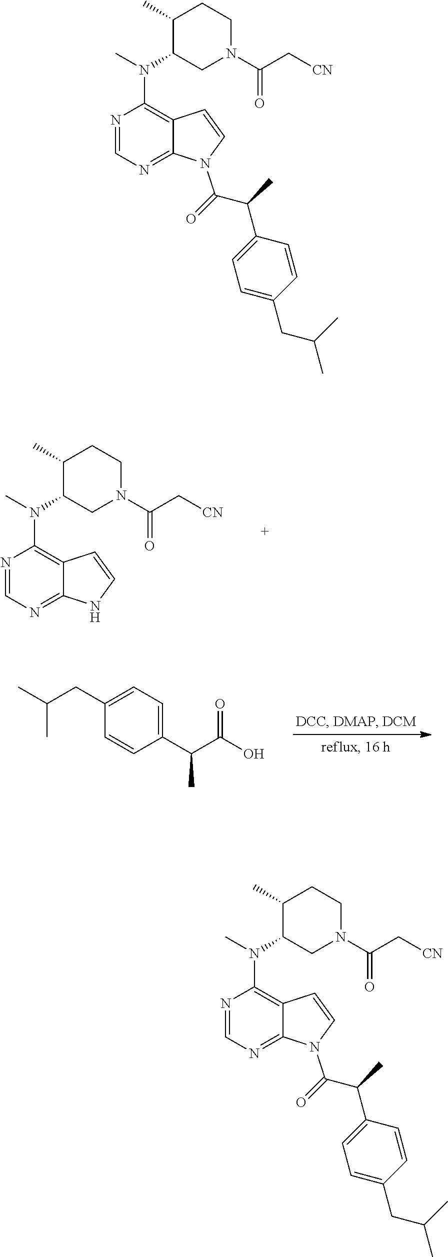 [SMILES: CC(C)Cc1ccc([C@H](C)C(=O)n2ccc3c(N(C)[C@H]4CN(C(=O)CC#N)CC[C@H]4C)ncnc32)cc1 ; C[C@@H]1CCN(C(=O)CC#N)C[C@@H]1N(C)c1ncnc2[nH]ccc12 ; "+" ; CC(C)Cc1ccc([C@H](C)C(=O)O)cc1 ; CC(C)Cc1ccc([C@H](C)C(=O)n2ccc3c(N(C)[C@H]4CN(C(=O)CC#N)CC[C@H]4C)ncnc32)cc1]

### Synthesis of 3-((3R, 4R)-3-((7-((S)-2-(4-isobutylphenyl) propanoyl)-7H-pyrrolo [2, 3-d] pyrimidin-4-yl) (methyl) amino)-4-methylpiperidin-1-yl)-3-oxopropanenitrile

3-{(3R, 4R)-4-methyl-3-[methyl-(7H-pyrrolo [2, 3-d] pyrimidin-4-yl)-piperidin-1-yl]-3-oxo-propionitrile (tofacitinib, 312 mg, 1 mmol) and S)-(+)-2-(4-isobutylphenyl) propanoic acid ((S)-(+)-ibuprofen, 309 mg, 1.5 mmol) were dissolved in dichloromethane (20 mL). 4-Dimethylaminopyridine (DMAP, 134 mg, 1.1 mmol) and dicyclohexyl carbodiimide (DCC, 412 mg, 2 mmol) were added in an ice water bath, followed by stirring at reflux for 16 hours. After completion of the reaction, the reaction solution was filtered. The filtrate was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 2:1) to give the title compound as a white solid, 0.24 g, 48% yield. MS (m/z): [M+H]⁺ calcd for C₂₉H₃₆N₆O₂, 501.65; found, 501.2. ¹H NMR (400 MHz, DMSO-d6) δ 8.38 (d, J=4.6 Hz, 1H), 7.68 (dd, J=4.3, 2.8 Hz, 1H), 7.29 (dd, J=8.2, 2.6 Hz, 2H), 7.06 (d, J=7.9 Hz, 2H), 6.88 (d, J=4.2 Hz, 1H), 6.09 (qd, J=6.7, 3.0 Hz, 1H), 4.85 (s, 1H), 4.18-3.59 (m, 5H), 3.41 (q, J=5.3, 4.9 Hz, 1H), 3.24 (d, J=2.4 Hz, 3H), 2.36 (d, J=7.2 Hz, 3H), 1.87-1.65 (m, 2H), 1.63-1.48 (m, 4H), 1.00 (dd, J=7.2, 2.6 Hz, 3H), 0.81 (d, J=6.6 Hz, 6H).

### Example 3

### (4-((3R, 4R)-1-(2-cyanoacetyl)-4-methylpiperidin-3-yl) (methyl) amino)-7H-pyrrolo [2, 3-d] pyrimidin-7-yl) methyl (S)-2-(4-isobutylphenyl) propanoate

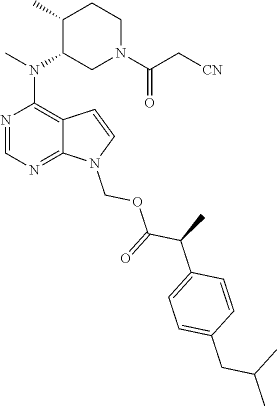 [SMILES: CC(C)Cc1ccc([C@H](C)C(=O)OCn2ccc3c(N(C)[C@H]4CN(C(=O)CC#N)CC[C@H]4C)ncnc32)cc1]

### First Step: Synthesis of 3-((3R, 4R)-4-methyl-3-(methyl (7-(2-(trimethylsilyl) ethoxy) methyl)-7H-pyrrolo [2, 3-d] pyrimidin-4-yl) amino) piperidin-1-yl)-3-oxopropanenitrile

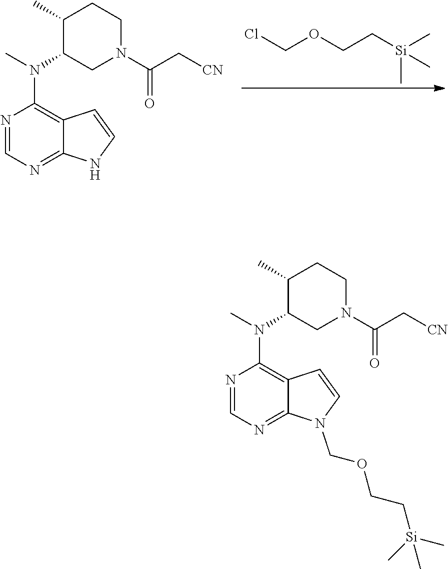

3-{(3R, 4R)-4-methyl-3-[methyl-(7H-pyrrolo [2, 3-d] pyrimidin-4-yl)-piperidin-1-yl]-3-oxo-propionitrile (tofacitinib, 9 g, 28.81 mmol) was dissolved in dichloromethane (180 mL) and diethyl acetate (3.745 g, 28.81 mol) under nitrogen protection. After stirring at room temperature for half an hour, (2-(chloromethoxy) ethyl) trimethylsilane (4.8 g, 28.81 mmol) was added and the stirring was continued at room temperature overnight. The solvent was evaporated under reduced pressure to give the crude product. The crude product was further isolated by silica gel column chromatography to give the title compound as a white solid, 9 g, 710% yield. MS (m/z): [M+H]⁺ calcd for C₂₂H₃₄N₆O₂Si, 443.25; found, 443.2.

### Second Step: Synthesis of 3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile

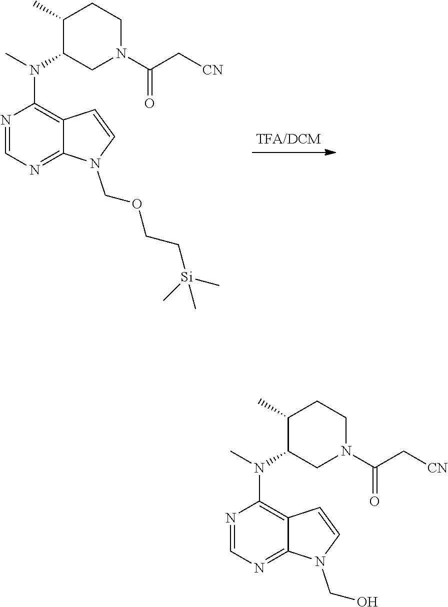 [SMILES: C[C@@H]1CCN(C(=O)CC#N)C[C@@H]1N(C)c1ncnc2c1ccn2COCC[Si](C)(C)C ; C[C@@H]1CCN(C(=O)CC#N)C[C@@H]1N(C)c1ncnc2c1ccn2CO]

Trifluoroacetic acid (6.44 g, 56.5 mmol) was slowly added dropwise to a solution of 3-((3R, 4R)-4-methyl-3-(methyl (7-(2-(trimethylsilyl) ethoxy) methyl)-7H-pyrrolo [2, 3-d] pyrimidin-4-yl) amino) piperidin-1-yl)-3-oxopropanenitrile (5 g, 11.3 mmol) in dichloromethane (100 mL) under nitrogen protection in an ice-water bath. After half an hour, the ice-water bath was removed and the temperature was raised to room temperature and stirring was continued for 24 hours. Saturated sodium bicarbonate solution was added to the above reaction solution at 0° C. to adjust the pH to 8. Then, the mixture was poured into a separation funnel and separated. The organic layer was washed with a saturated salt water solution, and dried over anhydrous sodium sulfate. After filtration, the solvent was evaporated under reduced pressure to give the title product 3.5 g, 90% yield. MS (m/z): [M+H]⁺ calcd for C₁₇H₂₂N₆O₂, 343.18; found, 343.1.

### Third Step: Synthesis of (4-((3R, 4R)-1-(2-cyanoacetyl)-4-methylpiperidin-3-yl) (methyl) amino)-7H-pyrrolo [2, 3-d] pyrimidin-7-yl) methyl (S)-2-(4-isobutylphenyl) propanoate

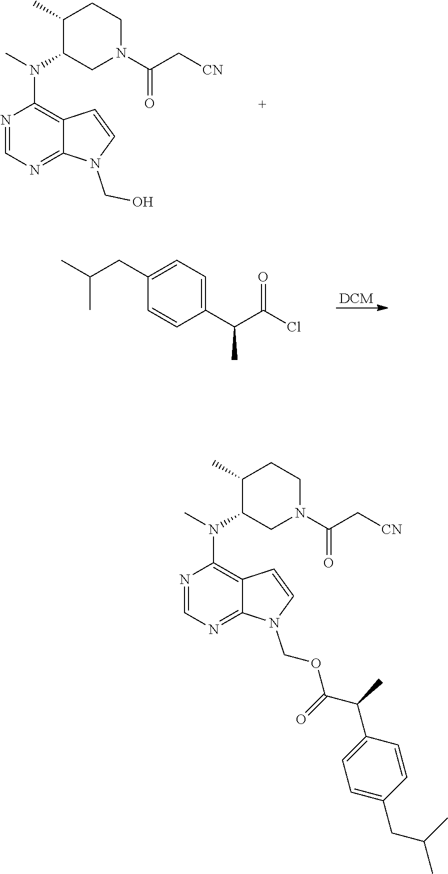

3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile (6.63 g, 19.4 mmol), (S)-2-(4-isobutylphenyl) propanoyl chloride (8.72 g, 38.8 mmol) and triethylamine (3.93 g, 38.8 mmol) were dissolved in dichloromethane (100 mL). After stirring at room temperature for 24 hours, the reaction mixture was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:1) to give the title compound, 4.05 g, 39.4% yield. MS (m/z): [M+H]⁺ calcd for C₃₀H₃₈N₆O₃, 531.67; found, 531.2. ¹H NMR (400 MHz, CDCl₃) δ 8.32 (d, J=7.4 Hz, 1H), 7.11 (dd, J=12.7, 5.5 Hz, 3H), 7.03 (d, J=7.6 Hz, 2H), 6.51 (s, 1H), 6.18 (dd, J=10.4, 4.0 Hz, 1H), 6.14-6.06 (m, 1H), 5.13 (s, 1H), 4.06 (dd, J=13.2, 3.8 Hz, 1H), 3.81 (dd, J=18.3, 10.5 Hz, 1H), 3.69 (q, J=7.1 Hz, 1H), 3.61 (t, J=11.8 Hz, 1H), 3.55-3.47 (m, 2H), 3.37 (d, J=17.9 Hz, 3H), 2.58-2.46 (m, 1H), 2.42 (d, J=7.1 Hz, 2H), 2.05-1.91 (m, 1H), 1.83 (td, J=13.0, 6.2 Hz, 2H), 1.45 (d, J=7.1 Hz, 3H), 1.28 (s, 1H), 1.09 (dd, J=12.9, 7.1 Hz, 3H), 0.88 (d, J=6.6 Hz, 6H).

### Example 4

### 3-((3R, 4R)-3-((7-(2-((2, 3-dimethylphenyl) amino) benzoyl)-7H-pyrrolo [2, 3-d] pyrimidin-4-yl) (methyl) amino)-4-methylpiperidin-1-yl)-3-oxopropanenitrile

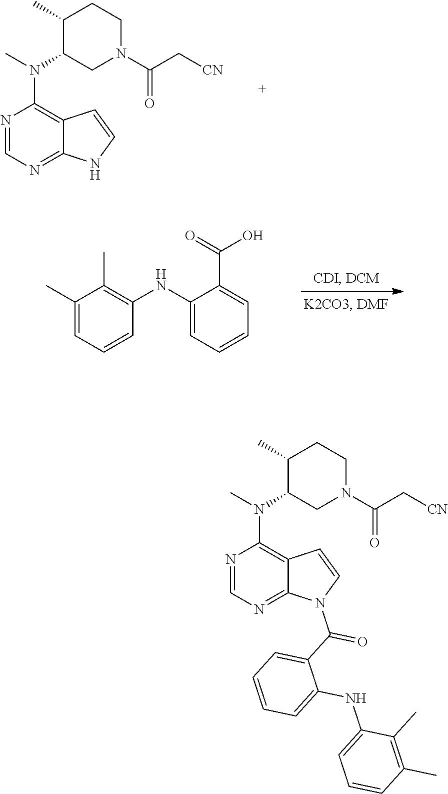 [SMILES: C[C@@H]1CCN(C(=O)CC#N)C[C@@H]1N(C)c1ncnc2[nH]ccc12 ; "+" ; Cc1cccc(Nc2ccccc2C(=O)O)c1C ; Cc1cccc(Nc2ccccc2C(=O)n2ccc3c(N(C)[C@H]4CN(C(=O)CC#N)CC[C@H]4C)ncnc32)c1C]

3-{(3R, 4R)-4-methyl-3-[methyl-(7H-pyrrolo [2, 3-d] pyrimidin-4-yl)-piperidin-1-yl]-3-oxo-propionitrile (tofacitinib, 312 mg, 1 mmol), 4-dimethylamino pyridine (DMAP, 12 mg, 0.1 mmol), 2-((2, 3-dimethylphenyl) amino) benzoic acid (mefenamic acid, 314 mg, 1.3 mmol) and 1-(3-dimethylaminopropyl)-3-ethylcarbodiimide hydrochloride (EDCI, 288 mg, 1.5 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further purified by silica gel column chromatography (petroleum ether/ethyl acetate=2:1 to 1:1) to give the title compound as a yellow solid, 0.1 g, yield 18.6%. MS (m/z): [M+H]⁺ calcd for C₃₁H₃₃N₇O₂, 536.65; found, 536.2.1H NMR (400 MHz, DMSO) δ 8.60 (d, J=16.4 Hz, 1H), 8.14 (d, J=7.5 Hz, 1H), 7.55-7.43 (m, 1H), 7.35 (dt, J=17.9, 8.5 Hz, 2H), 7.06 (d, J=6.2 Hz, 2H), 6.98 (s, 1H), 6.87 (t, J=12.4 Hz, 1H), 6.80 (d, J=8.4 Hz, 1H), 6.73 (t, J=7.4 Hz, 1H), 4.86 (s, 1H), 4.20-3.90 (m, 3H), 3.87-3.64 (m, 2H), 3.42 (s, 1H), 3.30 (s, 3H), 2.39 (d, J=5.3 Hz, 1H), 2.26 (s, 3H), 2.10-1.96 (m, 3H), 1.90-1.67 (m, 1H), 1.66-1.53 (m, 1H), 1.03 (d, J=6.9 Hz, 3H).

### Example 5

### 3-((3R, 4R)-3-((7-(2-((3-chloro-2-methylphenyl) amino) benzoyl)-7H-pyrrolo [2, 3-d] pyrimidin-4-yl) (methyl) amino)-4-methylpiperidin-1-yl)-3-oxopropanenitrile

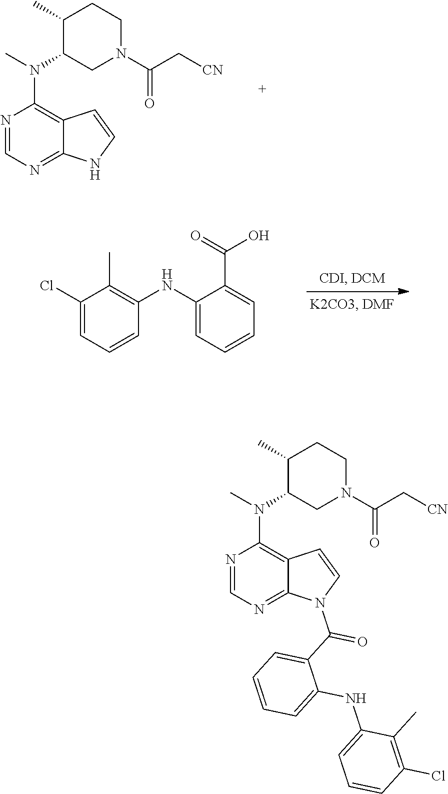 [SMILES: C[C@@H]1CCN(C(=O)CC#N)C[C@@H]1N(C)c1ncnc2[nH]ccc12 ; "+" ; Cc1c(Cl)cccc1Nc1ccccc1C(=O)O ; Cc1c(Cl)cccc1Nc1ccccc1C(=O)n1ccc2c(N(C)[C@H]3CN(C(=O)CC#N)CC[C@H]3C)ncnc21]

3-{(3R, 4R)-4-methyl-3-[methyl-(7H-pyrrolo [2, 3-d] pyrimidin-4-yl)-piperidin-1-yl]-3-oxo-propionitrile (tofacitinib, 187 mg, 0.6 mmol), 4-dimethylamino pyridine (DMAP, 7 mg, 0.6 mmol), 2-((3-chloro-2-methylphenyl) amino) benzoic acid (tofenamic acid, 204 mg, 0.78 mmol) and 1-(3-dimethylaminopropyl)-3-ethylcarbodiimide hydrochloride (EDCI, 173 mg, 0.9 mmol) was dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further purified by silica gel column chromatography (petroleum ether/ethyl acetate=2:1 to 1:1) to give the title compound as a yellow solid, 0.12 g, 35.9% yield. MS (m/z): [M+H]⁺ calcd for C₃₀H₃₀ClN₇O₂, 557.07; found, 557.2. ¹H NMR (400 MHz, DMSO-d6) δ 8.23 (d, J=17.2 Hz, 1H), 8.10 (d, J=8.3 Hz, 1H), 7.46 (ddd, J=8.6, 7.1, 1.6 Hz, 1H), 7.43-7.34 (m, 2H), 7.07 (tq, J=7.0, 4.8, 3.6 Hz, 3H), 6.96 (d, J=8.3 Hz, 1H), 6.90 (t, J=7.5 Hz, 1H), 6.84 (d, J=4.1 Hz, 1H), 4.84 (s, 1H), 4.19-3.90 (m, 3H), 3.74 (dtd, J=34.8, 14.2, 13.1, 7.4 Hz, 2H), 3.50-3.39 (m, 1H), 3.29-3.21 (m, 3H), 2.46-2.32 (m, 1H), 2.06 (d, J=8.5 Hz, 3H), 1.92-1.66 (m, 1H), 1.64-1.51 (m, 1H), 1.03 (d, J=7.1 Hz, 3H).

### Example 6

### 3-((3R, 4R)-3-((7-((S)-2-(6-methoxynaphthalen-2-yl) propanoyl)-7H-pyrrolo [2, 3-d] pyrimidin-4-yl) (methyl) amino)-4-methylpiperidin-1-yl)-3-oxopropanenitrile

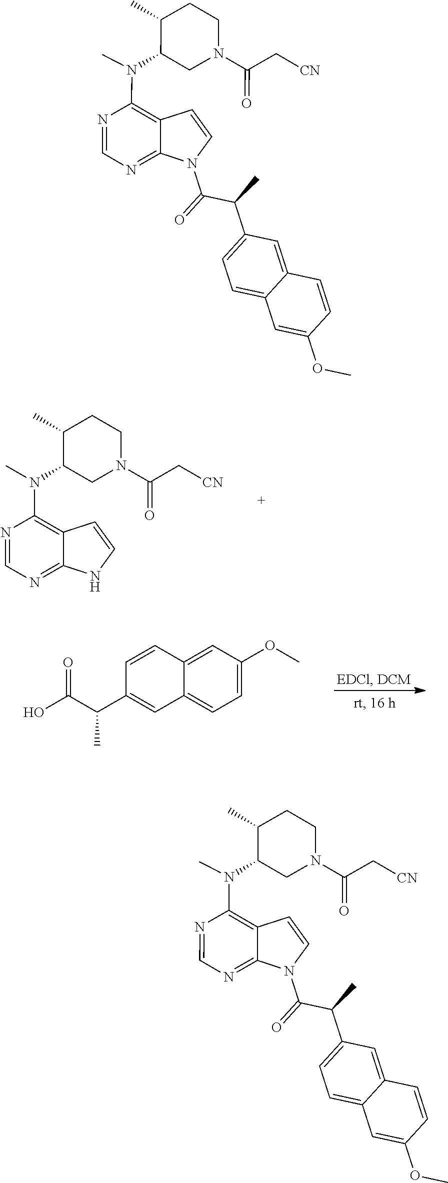

### Synthesis of 3-((3R, 4R)-3-((7-((S)-2-(6-methoxynaphthalen-2-yl) propanoyl)-7H-pyrrolo [2, 3-d] pyrimidin-4-yl) (methyl) amino)-4-methylpiperidin-1-yl)-3-oxopropanenitrile

3-{(3R, 4R)-4-methyl-3-[methyl-(7H-pyrrolo [2, 3-d] pyrimidin-4-yl)-piperidin-1-yl]-3-oxo-propionitrile (tofacitinib, 624 mg, 2 mmol), (S)-(+)-2-(6-methoxy-2-naphthyl) propanoic acid (naproxen, 506 mg, 2.2 mmol) and 1-(3-dimethylaminopropyl)-3-ethylcarbodiimide hydrochloride (EDCI, 863 mg, 4.5 mmol) were dissolved in dichloromethane (40 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=1:1 to 1:2) to give the title compound as a white solid, 0.7 g, 66.8% yield. MS (m/z): [M+H]⁺ calcd for C₃₀H₃₂N₆O₃, 525.63; found, 525.3. ¹H NMR (400 MHz, DMSO) δ 8.41 (s, 1H), 7.81-7.69 (m, 4H), 7.50 (dd, J=7.0, 5.3 Hz, 1H), 7.24 (s, 1H), 7.12 (dd, J=8.9, 2.3 Hz, 1H), 6.88 (d, J=3.7 Hz, 1H), 6.25-6.16 (m, 1H), 4.83 (s, 1H), 4.17-3.87 (m, 3H), 3.84 (s, 3H), 3.72-3.59 (m, 2H), 3.41-3.39 (m, 1H), 3.23 (s, 3H), 2.32 (d, J=13.3 Hz, 1H), 1.83-1.49 (m, 5H), 0.99 (d, J=7.1 Hz, 3H)

### Example 7

3-((3R, 4R)-3-((7-(2-(3-benzoylphenyl) propanoyl)-7H-pyrrolo [2, 3-d] pyrimidin-4-yl) (methyl) amino)-4-methylpiperidin-1-yl)-3-oxopropanenitrile

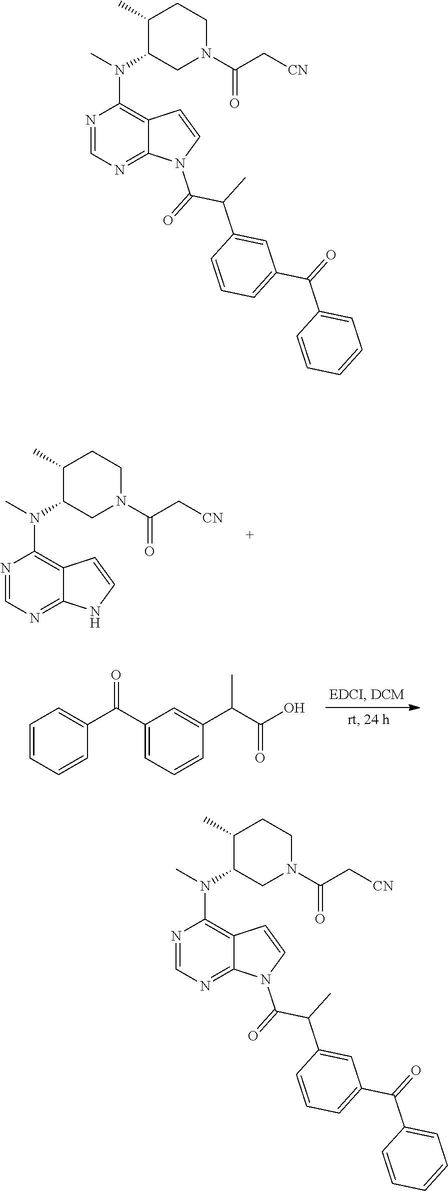 [SMILES: CC(C(=O)n1ccc2c(N(C)[C@H]3CN(C(=O)CC#N)CC[C@H]3C)ncnc21)c1cccc(C(=O)c2ccccc2)c1 ; C[C@@H]1CCN(C(=O)CC#N)C[C@@H]1N(C)c1ncnc2[nH]ccc12 ; CC(C(=O)O)c1cccc(C(=O)c2ccccc2)c1 ; CC(C(=O)n1ccc2c(N(C)[C@H]3CN(C(=O)CC#N)CC[C@H]3C)ncnc21)c1cccc(C(=O)c2ccccc2)c1]

### Synthesis of 3-((3R, 4R)-3-((7-(2-(3-benzoylphenyl) propanoyl)-7H-pyrrolo [2, 3-d] pyrimidin-4-yl) (methyl) amino)-4-methylpiperidin-1-yl)-3-oxopropanenitrile

3-{(3R, 4R)-4-methyl-3-[methyl-(7H-pyrrolo [2, 3-d] pyrimidin-4-yl)-piperidin-1-yl]-3-oxo-propionitrile (tofacitinib, 624 mg, 2 mmol), 2-(3-benzoylphenyl) propanoic acid (ketoprofen, 559 mg, 2.2 mmol) and 1-(3-dimethylaminopropyl)-3-ethylcarbodiimide hydrochloride (EDCI, 576 mg, 3 mmol) were dissolved in dichloromethane (20 mL) and stirred at room temperature for 24 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:2) to give the title compound as a white solid, 0.73 g, 66.6% yield. MS (m/z): [M+H]⁺ calcd for C₃₂H₃₂N₆O₃, 549.65; found, 549.3. ¹H NMR (400 MHz, DMSO) δ 8.33 (d, J=7.4 Hz, 1H), 7.79 (d, J=1.6 Hz, 1H), 7.76-7.63 (m, 5H), 7.61 (d, J=7.8 Hz, 1H), 7.57 (d, J=8.5 Hz, 1H), 7.54 (s, 1H), 7.51 (d, J=7.7 Hz, 1H), 6.94 (d, J=3.8 Hz, 1H), 6.16 (q, J=6.8 Hz, 1H), 4.85 (s, 1H), 4.14-3.90 (m, 3H), 3.83-3.60 (m, 2H), 3.46-3.39 (m, 1H), 3.26 (s, 3H), 2.44-2.29 (m, 1H), 1.90-1.68 (m, 1H), 1.67-1.51 (m, 4H), 1.02 (t, J=9.4 Hz, 3H).

### Example 8

### 3-((3R, 4R)-3-((7-(2-(2-fluoro-[1, 1′-biphenyl]-4-yl) propanoyl)-7H-pyrrolo [2, 3-d] pyrimidin-4-yl) (methyl) amino)-4-methylpiperidin-1-yl)-3-oxopropanenitrile

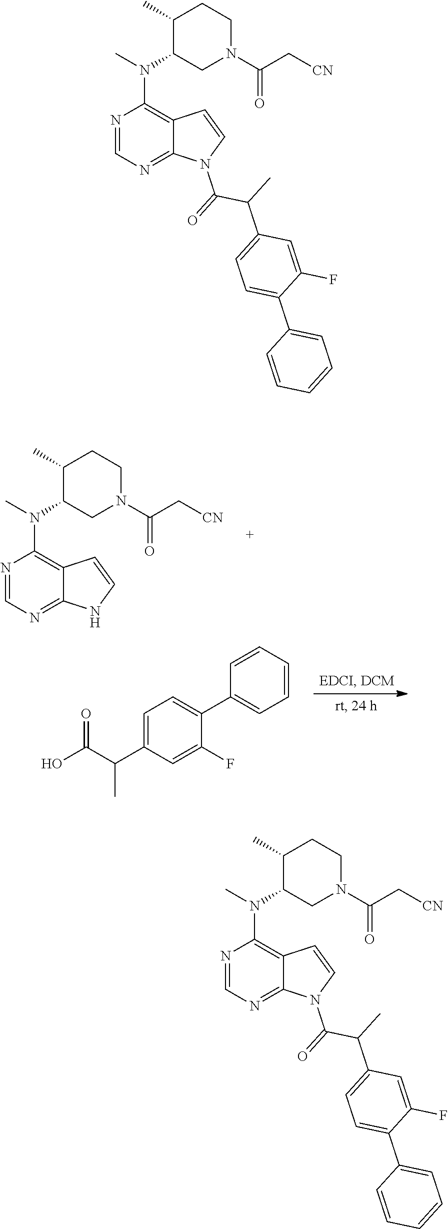 [SMILES: CC(C(=O)n1ccc2c(N(C)[C@H]3CN(C(=O)CC#N)CC[C@H]3C)ncnc21)c1ccc(-c2ccccc2)c(F)c1 ; C[C@@H]1CCN(C(=O)CC#N)C[C@@H]1N(C)c1ncnc2[nH]ccc12 ; CC(C(=O)O)c1ccc(-c2ccccc2)c(F)c1 ; CC(C(=O)n1ccc2c(N(C)[C@H]3CN(C(=O)CC#N)CC[C@H]3C)ncnc21)c1ccc(-c2ccccc2)c(F)c1]

### Synthesis of 3-((3R, 4R)-3-((7-(2-(2-fluoro-[1, 1′-biphenyl]-4-yl) propanoyl)-7H-pyrrolo [2, 3-d] pyrimidin-4-yl) (methyl) amino)-4-methylpiperidin-1-yl)-3-oxopropanenitrile

3-{(3R, 4R)-4-methyl-3-[methyl-(7H-pyrrolo [2, 3-d] pyrimidin-4-yl)-piperidin-1-yl]-3-oxo-propionitrile (tofacitinib, 624 mg, 2 mmol), 2-(2-fluoro-4-biphenyl) propanoic acid (flurbiprofen, 537 mg, 2.2 mmol) and 1-(3-dimethylaminopropyl)-3-ethylcarbodiimide hydrochloride (EDCI, 576 mg, 3 mmol) were dissolved in dichloromethane (20 mL) and stirred at room temperature for 24 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=1:1 to 1:2) to give the title compound as a white solid, 0.73 g, 67.8% yield. MS (m/z): [M+H]⁺ calcd for C₃₁H₃₁FN₆O₂, 539.63; found, 539.2. ¹H NMR (400 MHz, DMSO) δ 8.41 (dd, J=5.7, 1.3 Hz, 1H), 7.72 (d, J=4.2 Hz, 1H), 7.54-7.43 (m, 5H), 7.41-7.27 (m, 3H), 6.93 (s, 1H), 6.16 (q, J=6.7 Hz, 1H), 4.84 (s, 1H), 4.16-3.99 (m, 2H), 3.98-3.87 (m, 1H), 3.75-3.59 (m, 2H), 3.45-3.39 (m, 1H), 3.23 (s, 3H), 2.43-2.27 (m, 1H), 1.88-1.66 (m, 1H), 1.65-1.52 (m, 4H), 1.04-0.93 (m, 3H).

### Example 9

### 3-((3R, 4R)-3-((7-(2-(2-(2-(2-(2, 6-dichlorophenyl) amino) phenyl) acetyl)-7H-pyrrolo [2, 3-d] pyrimidin-4-yl) (methyl) amino)-4-methylpiperidin-1-yl)-3-oxopropanenitrile

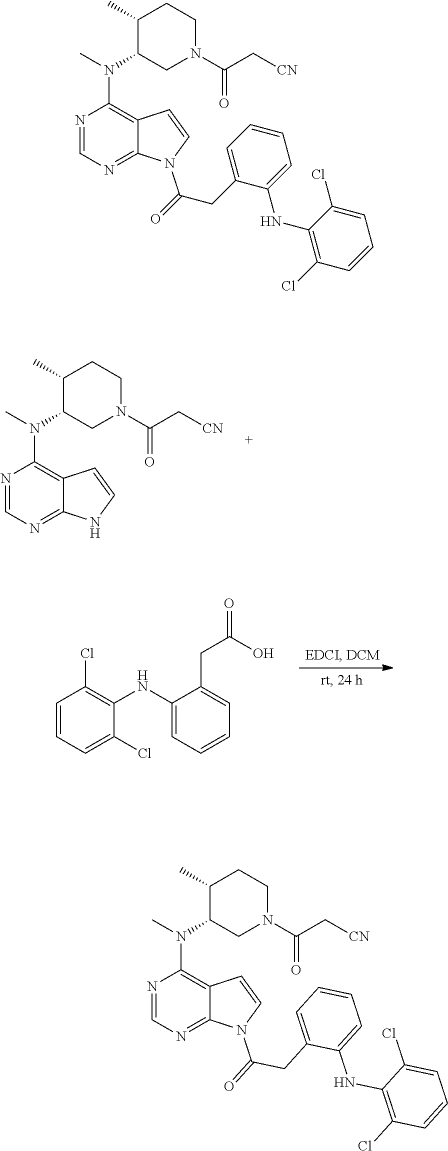 [SMILES: C[C@@H]1CCN(C(=O)CC#N)C[C@@H]1N(C)c1ncnc2c1ccn2C(=O)Cc1ccccc1Nc1c(Cl)cccc1Cl ; C[C@@H]1CCN(C(=O)CC#N)C[C@@H]1N(C)c1ncnc2[nH]ccc12 ; O=C(O)Cc1ccccc1Nc1c(Cl)cccc1Cl ; C[C@@H]1CCN(C(=O)CC#N)C[C@@H]1N(C)c1ncnc2c1ccn2C(=O)Cc1ccccc1Nc1c(Cl)cccc1Cl]

### Synthesis of 3-((3R, 4R)-3-((7-(2-(2-(2-(2-(2, 6-dichlorophenyl) amino) phenyl) acetyl)-7H-pyrrolo [2, 3-d] pyrimidin-4-yl) (methyl) amino)-4-methylpiperidin-1-yl)-3-oxopropanenitrile

3-{(3R, 4R)-4-methyl-3-[methyl-(7H-pyrrolo [2, 3-d] pyrimidin-4-yl)-piperidin-1-yl]-3-oxo-propionitrile (tofacitinib, 624 mg, 2 mmol), 2-(2, 6-dichlorophenylamino) phenylacetic acid (diclofenac, 651 mg, 2.2 mmol) and 1-(3-dimethylaminopropyl)-3-ethylcarbodiimide hydrochloride (EDCI, 576 mg, 3 mmol) were dissolved in dichloromethane (6 mL) and stirred at room temperature for 24 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:2) to give the title compound as a white solid, 0.25 g, 21.2% yield. MS (m/z): [M+H]⁺ calcd for C₃₀H₂₉Cl₂N₇O₂, 590.51; found, 590.2. ¹H NMR (400 MHz, DMSO) δ 8.41 (d, J=5.9 Hz, 1H), 7.75 (d, J=4.1 Hz, 1H), 7.51 (d, J=8.1 Hz, 2H), 7.35 (d, J=12.7 Hz, 1H), 7.28 (d, J=7.0 Hz, 1H), 7.20 (t, J=8.1 Hz, 1H), 7.07 (t, J=7.7 Hz, 1H), 6.96 (d, J=3.8 Hz, 1H), 6.83 (t, J=7.4 Hz, 1H), 6.22 (d, J=8.0 Hz, 1H), 4.99-4.83 (m, 3H), 4.16-3.99 (m, 3H), 3.88-3.61 (m, 2H), 3.40 (d, J=14.4 Hz, 1H), 3.29 (s, 3H), 2.45-2.29 (m, 1H), 1.89-1.67 (m, 1H), 1.65-1.52 (m, 1H), 1.01 (d, J=7.1 Hz, 3H).

### Example 10

### 3-((3R, 4R)-4-methyl-3-(methyl (7-(2-(3-phenoxyphenyl) propanoyl)-7H-pyrrolo [2, 3-d] pyrimidin-4-yl) amino) piperidin-1-yl)-3-oxopropanenitrile

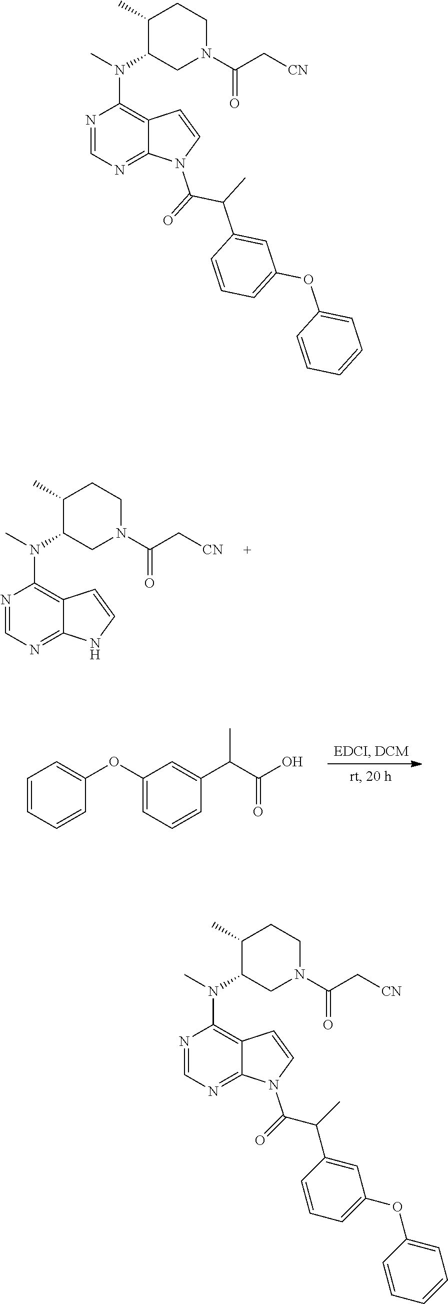 [SMILES: CC(C(=O)n1ccc2c(N(C)[C@H]3CN(C(=O)CC#N)CC[C@H]3C)ncnc21)c1cccc(Oc2ccccc2)c1 ; C[C@@H]1CCN(C(=O)CC#N)C[C@@H]1N(C)c1ncnc2[nH]ccc12 ; CC(C(=O)O)c1cccc(Oc2ccccc2)c1 ; CC(C(=O)n1ccc2c(N(C)[C@H]3CN(C(=O)CC#N)CC[C@H]3C)ncnc21)c1cccc(Oc2ccccc2)c1]

### Synthesis of 3-((3R, 4R)-4-methyl-3-(methyl (7-(2-(3-phenoxyphenyl) propanoyl)-7H-pyrrolo [2, 3-d] pyrimidin-4-yl) amino) piperidin-1-yl)-3-oxopropanenitrile

3-{(3R, 4R)-4-methyl-3-[methyl-(7H-pyrrolo [2, 3-d] pyrimidin-4-yl)-piperidin-1-yl]-3-oxo-propionitrile (tofacitinib, 781 mg, 2.5 mmol), 2-(3-phenoxyphenyl) propanoic acid (fenoprofen, 787.4 mg, 3.25 mmol) and 1-(3-dimethylaminopropyl)-3-ethylcarbodiimide hydrochloride (EDCI, 720.5 mg, 3.75 mmol) were dissolved in dichloromethane (20 mL) and stirred at room temperature for 20 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:2) to give the title compound as a white solid, 1.1 g, 82% yield. MS (m/z): [M+H]⁺ calcd for C₃₁H₃₂N₆O₃ 537.64; found, 537.3. ¹H NMR (400 MHz, Chloroform-d) δ 8.30 (dd, J=10.4, 4.8 Hz, 1H), 7.77-7.61 (m, 1H), 7.36-7.16 (m, 5H), 7.10 (t, J=7.4 Hz, 1H), 6.98-6.92 (m, 2H), 6.83 (dt, J=7.7, 2.0 Hz, 1H), 6.62 (t, J=4.2 Hz, 1H), 6.10 (qd, J=7.0, 4.5 Hz, 1H), 5.09 (ddq, J=14.2, 9.9, 4.8 Hz, 1H), 4.12-3.67 (m, 2H), 3.63-3.41 (m, 4H), 3.33 (d, J=15.6 Hz, 3H), 2.48 (ddt, J=17.9, 12.9, 5.9 Hz, 1H), 1.93 (dddd, J=17.4, 12.6, 7.5, 3.9 Hz, 1H), 1.81-1.68 (m, 1H), 1.66-1.58 (m, 3H), 1.08 (dd, J=12.6, 7.1 Hz, 3H).

### Example 11

### 3-((3R, 4R)-4-methyl-3-(methyl (7-(2-(4-((2-oxocyclopentyl) methyl) phenyl) propanoyl)-7H-pyrrolo [2, 3-d] pyrimidin-4-yl) amino) piperidin-1-yl)-3-oxopropanenitrile

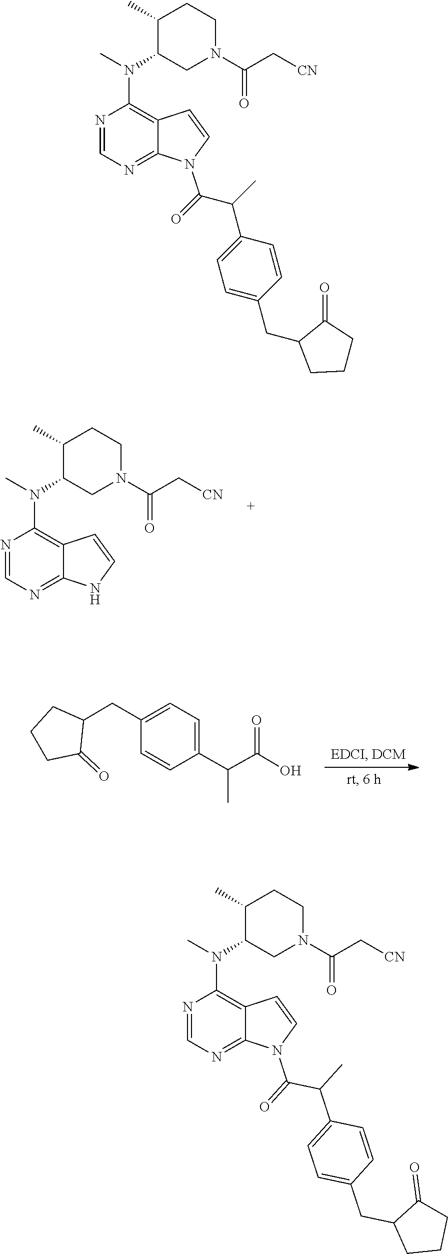 [SMILES: CC(C(=O)n1ccc2c(N(C)[C@H]3CN(C(=O)CC#N)CC[C@H]3C)ncnc21)c1ccc(CC2CCCC2=O)cc1 ; C[C@@H]1CCN(C(=O)CC#N)C[C@@H]1N(C)c1ncnc2[nH]ccc12 ; CC(C(=O)O)c1ccc(CC2CCCC2=O)cc1 ; CC(C(=O)n1ccc2c(N(C)[C@H]3CN(C(=O)CC#N)CC[C@H]3C)ncnc21)c1ccc(CC2CCCC2=O)cc1]

### Synthesis of 3-((3R, 4R)-4-methyl-3-(methyl (7-(2-(4-((2-oxocyclopentyl) methyl) phenyl) propanoyl)-7H-pyrrolo [2, 3-d] pyrimidin-4-yl) amino) piperidin-1-yl)-3-oxopropanenitrile

3-{(3R, 4R)-4-methyl-3-[methyl-(7H-pyrrolo [2, 3-d] pyrimidin-4-yl)-piperidin-1-yl]-3-oxo-propionitrile (tofacitinib, 624 mg, 2 mmol), 2-[4-(2-oxocyclopentan-1-ylmethyl) phenyl] propanoic acid (loxoprofen, 541 mg, 2.2 mmol) and 1-(3-dimethylaminopropyl)-3-ethylcarbodiimide hydrochloride (EDCI, 576 mg, 3 mmol) were dissolved in dichloromethane (20 mL) and stirred at room temperature for 6 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:2) to give the title compound as a white solid, 0.48 g, 44.4% yield. MS (m/z): [M+H]⁺ calcd for C₃₁H₃₆N₆O₃, 541.67; found, 541.3. ¹H NMR (400 MHz, DMSO) δ 8.38 (dd, J=6.2, 1.5 Hz, 1H), 7.80-7.60 (m, 1H), 7.32 (dd, J=17.9, 16.2 Hz, 2H), 7.10 (d, J=7.9 Hz, 2H), 6.89 (d, J=4.3 Hz, 1H), 6.19-5.98 (m, 1H), 4.84 (s, 1H), 4.17-3.98 (m, 2H), 3.96-3.57 (m, 3H), 3.40 (t, J=5.9 Hz, 1H), 3.21 (s, 3H), 2.94-2.83 (m, 1H), 2.41-2.25 (m, 3H), 2.25-2.14 (m, 1H), 2.09-2.02 (m, 1H), 1.90-1.80 (m, 3H), 1.72-1.48 (m, 5H), 1.48-1.37 (m, 1H), 1.07-0.93 (m, 3H).

### Example 12

### 3-((3R, 4R)-3-((7-(2-(1-(4-chlorobenzoyl)-5-methoxy-2-methyl-1H-indol-3-yl) acetyl)-7H-pyrrolo [2, 3-d] pyrimidin-4-yl) (methyl) amino)-4-methylpiperidin-1-yl)-3-oxopropanenitrile

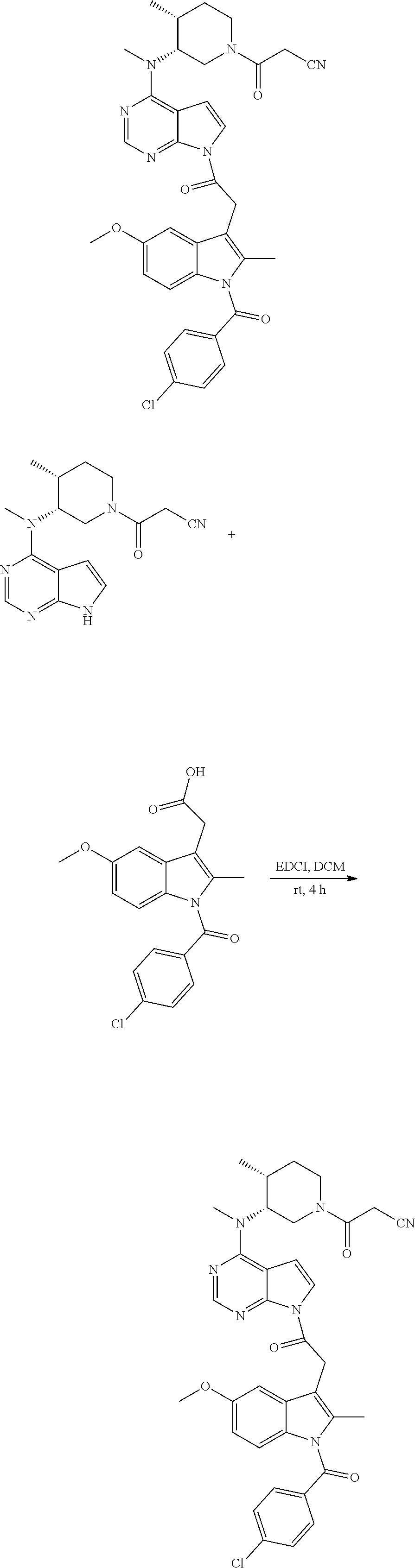

### Synthesis of 3-((3R, 4R)-3-((7-(2-(1-(4-chlorobenzoyl)-5-methoxy-2-methyl-1H-indol-3-yl) acetyl)-7H-pyrrolo [2, 3-d] pyrimidin-4-yl) (methyl) amino)-4-methylpiperidin-1-yl)-3-oxopropanenitrile

3-{(3R, 4R)-4-methyl-3-[methyl-(7H-pyrrolo [2, 3-d] pyrimidin-4-yl)-piperidin-1-yl]-3-oxo-propionitrile (tofacitinib, 156 mg, 0.5 mmol), 1-(4-chlorobenzoyl)-5-methoxy-2-methyl-1H-indole-3-acetic acid (indomethacin, 233 mg, 0.65 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 4 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 2:3) to give the title compound as a white solid, 0.16 g, 49.1% yield. MS (m/z): [M+H]⁺ calcd for C₃₅H₃₄ClN₇O₄, 653.15; found, 653.3. ¹H NMR (400 MHz, DMSO-d₆) δ 8.45 (d, J=6.4 Hz, 1H), 7.77-7.61 (m, 5H), 7.11 (t, J=2.8 Hz, 1H), 6.97 (t, J=6.7 Hz, 2H), 6.71 (dd, J=9.1, 2.5 Hz, 1H), 5.05-4.86 (m, 3H), 4.21-3.89 (m, 3H), 3.86-3.64 (m, 5H), 3.46-3.40 (m, 1H), 3.31 (s, 3H), 2.44-2.38 (m, 1H), 1.88-1.52 (m, 2H), 1.32-1.21 (m, 3H), 1.02 (d, J=7.1 Hz, 3H).

### Example 13

### N-(4-(2-(4-(((3R, 4R)-1-(2-cyanoacetyl)-4-methylpiperidin-3-yl) (methyl) amino)-7H-pyrrolo [2, 3-d] pyrimidin-7-yl)-2-oxoethyl) phenyl) acetamide

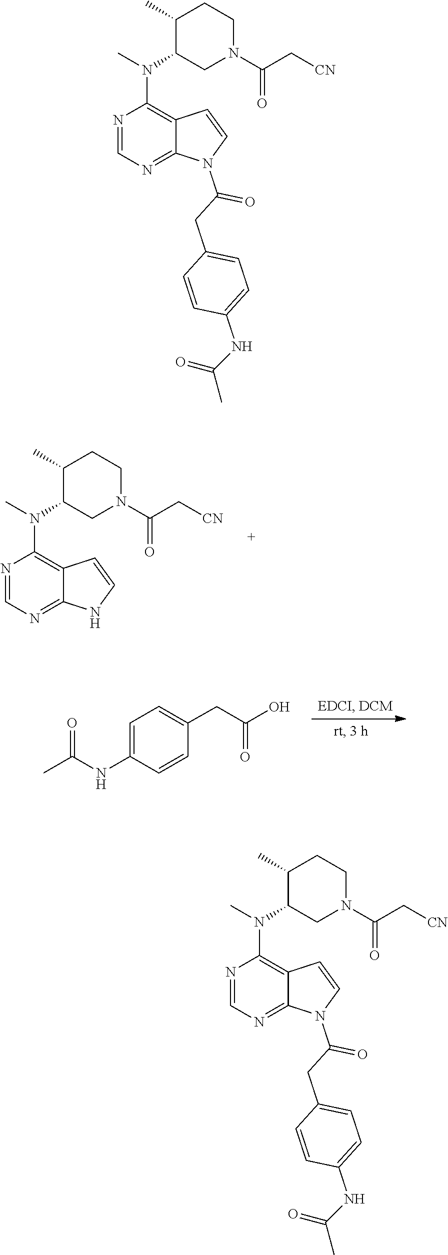 [SMILES: CC(=O)Nc1ccc(CC(=O)n2ccc3c(N(C)[C@H]4CN(C(=O)CC#N)CC[C@H]4C)ncnc32)cc1 ; C[C@@H]1CCN(C(=O)CC#N)C[C@@H]1N(C)c1ncnc2[nH]ccc12 ; CC(=O)Nc1ccc(CC(=O)O)cc1 ; CC(=O)Nc1ccc(CC(=O)n2ccc3c(N(C)[C@H]4CN(C(=O)CC#N)CC[C@H]4C)ncnc32)cc1]

### Synthesis of N-(4-(2-(4-(((3R, 4R)-1-(2-cyanoacetyl)-4-methylpiperidin-3-yl) (methyl) amino)-7H-pyrrolo [2, 3-d] pyrimidin-7-yl)-2-oxoethyl) phenyl) acetamide

3-{(3R, 4R)-4-methyl-3-[methyl-(7H-pyrrolo [2, 3-d] pyrimidin-4-yl)-piperidin-1-yl]-3-oxo-propionitrile (tofacitinib, 156 mg, 0.5 mmol), 2-(4-acetamidophenyl) acetic acid (actarit, 126 mg, 0.65 mmol) and 1-(3-dimethylaminopropyl)-3-ethylcarbodiimide hydrochloride (EDCI, 144 mg, 0.75 mmol) were dissolved in dichloromethane (8 mL) and stirred at room temperature for 3 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=1:1 to 1:6) to give the title compound as a white solid, 0.118 g, 48.6% yield. MS (m/z): [M+H]⁺ calcd for C₂₆H₂₉N₇O₃, 488.56; found, 488.2. ¹H NMR (400 MHz, DMSO-d₆) δ 9.90 (s, 1H), 8.39 (d, J=5.8 Hz, 1H), 7.70 (d, J=4.1 Hz, 1H), 7.51 (d, J=8.1 Hz, 2H), 7.26 (d, J=8.1 Hz, 2H), 6.94 (d, J=4.4 Hz, 1H), 4.97-4.71 (m, 3H), 4.24-3.89 (m, 3H), 3.85-3.59 (m, 1H), 3.49-3.40 (m, 1H), 3.28 (s, 3H), 2.39 (q, J=6.2 Hz, 1H), 2.03 (s, 3H), 1.88-1.46 (m, 2H), 1.02 (d, J=7.0 Hz, 3H).

### Example 14

### 3-((3R, 4R)-3-((7-(2-(1, 8-diethyl-1, 3, 4, 9-tetrahydropyrano [3, 4-b] indol-1-yl) acetyl)-7H-pyrrolo [2, 3-d] pyrimidin-4-yl) (methyl) amino)-4-methylpiperidin-1-yl)-3-oxopropanenitrile

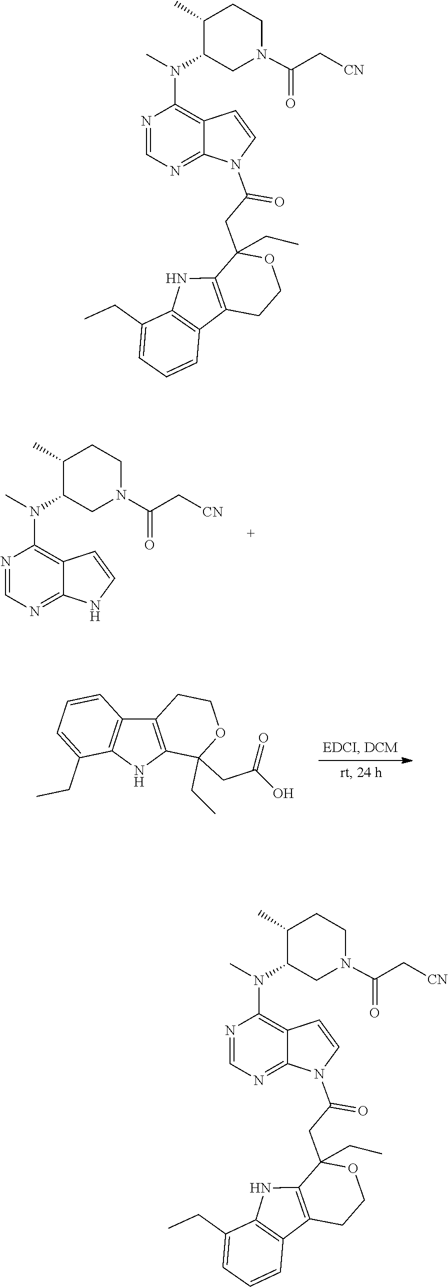 [SMILES: CCc1cccc2c3c([nH]c12)C(CC)(CC(=O)n1ccc2c(N(C)[C@H]4CN(C(=O)CC#N)CC[C@H]4C)ncnc21)OCC3 ; C[C@@H]1CCN(C(=O)CC#N)C[C@@H]1N(C)c1ncnc2[nH]ccc12 ; CCc1cccc2c3c([nH]c12)C(CC)(CC(=O)O)OCC3 ; CCc1cccc2c3c([nH]c12)C(CC)(CC(=O)n1ccc2c(N(C)[C@H]4CN(C(=O)CC#N)CC[C@H]4C)ncnc21)OCC3]

### Synthesis of 3-((3R, 4R)-3-((7-(2-(1, 8-diethyl-1, 3, 4, 9-tetrahydropyrano [3,4-b] indol-1-yl) acetyl)-7H-pyrrolo [2, 3-d] pyrimidin-4-yl) (methyl) amino)-4-methylpiperidin-1-yl)-3-oxopropanenitrile

3-{(3R, 4R)-4-methyl-3-[methyl-(7H-pyrrolo [2, 3-d] pyrimidin-4-yl)-piperidin-1-yl]-3-oxo-propionitrile (tofacitinib, 156 mg, 0.5 mmol), 1, 8-diethyl-1, 3, 4, 9-tetrahydropyran [3, 4-b] indole-1-acetic acid (etodolac, 158 mg, 0.55 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 24 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=1:1 to 1:2) to give the title compound as a white solid, 0.11 g, 37.8% yield. MS (m/z): [M+H]⁺ calcd for C₃₃H₃₉N₇O₃, 582.72; found, 582.3.1H NMR (400 MHz, DMSO-d₆) δ 10.50 (s, 1H), 8.29 (s, 1H), 7.67 (td, J=4.5, 2.2 Hz, 1H), 7.23 (dd, J=7.5, 2.0 Hz, 1H), 6.98-6.81 (m, 3H), 4.84 (s, 1H), 4.67 (ddd, J=14.7, 8.6, 6.3 Hz, 1H), 4.20-3.98 (m, 3H), 3.98-3.82 (m, 2H), 3.81-3.61 (m, 3H), 3.47-3.38 (m, 1H), 3.27 (s, 3H), 2.84 (q, J=7.5 Hz, 2H), 2.60 (dt, J=9.4, 4.7 Hz, 2H), 2.45-2.30 (m, 1H), 2.16 (qq, J=6.9, 4.5, 3.9 Hz, 2H), 1.83 (s, 2H), 1.26 (td, J=7.6, 3.1 Hz, 3H), 1.01 (dt, J=7.2, 2.8 Hz, 3H), 0.69 (td, J=7.1, 3.4 Hz, 3H).

### Example 15

### 2-(4-((3R, 4R)-1-(2-cyanoacetyl)-4-methylpiperidin-3-yl) (methyl) amino)-7H-pyrrolo [2, 3-d] pyrimidine-7-carbonyl) phenyl acetate

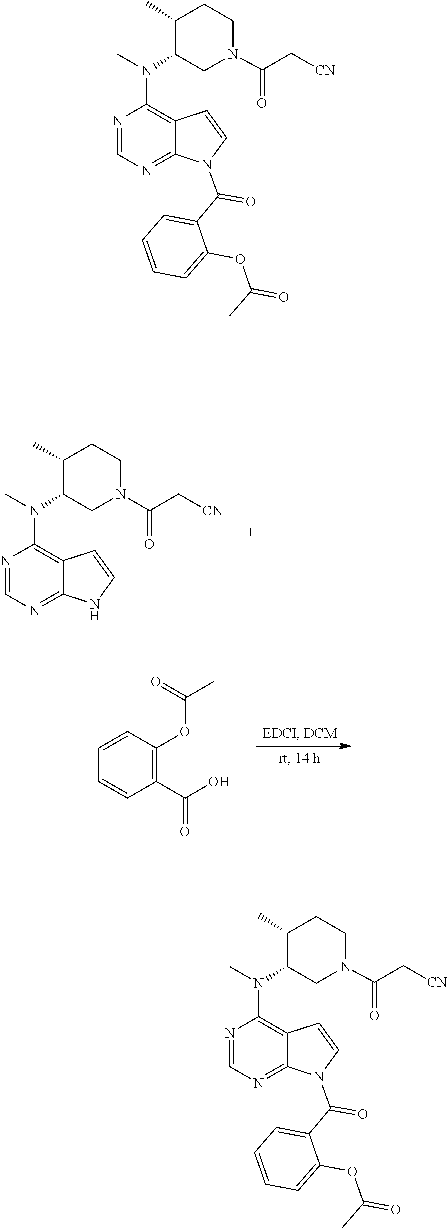 [SMILES: CC(=O)Oc1ccccc1C(=O)n1ccc2c(N(C)[C@H]3CN(C(=O)CC#N)CC[C@H]3C)ncnc21 ; C[C@@H]1CCN(C(=O)CC#N)C[C@@H]1N(C)c1ncnc2[nH]ccc12 ; CC(=O)Oc1ccccc1C(=O)O ; CC(=O)Oc1ccccc1C(=O)n1ccc2c(N(C)[C@H]3CN(C(=O)CC#N)CC[C@H]3C)ncnc21]

### Synthesis of 2-(4-((3R, 4R)-1-(2-cyanoacetyl)-4-methylpiperidin-3-yl) (methyl) amino)-7H-pyrrolo [2, 3-d] pyrimidine-7-carbonyl) phenyl acetate

3-{(3R, 4R)-4-methyl-3-[methyl-(7H-pyrrolo [2, 3-d] pyrimidin-4-yl)-piperidin-1-yl]-3-oxo-propionitrile (tofacitinib, 312 mg, 1 mmol), 2-acetoxybenzoic acid (aspirin, 216 mg, 1.2 mmol) and 1-(3-dimethylaminopropyl)-3-ethylcarbodiimide hydrochloride (EDCI, 288 mg, 1.5 mmol) were dissolved in dichloromethane (10 mL) and stirred at room temperature for 14 hours. After completion of the reaction, the reaction solution was diluted with dichloromethane and washed with water and saturated salt water solution.

The organic layer was dried over anhydrous sodium sulfate and filtered. The solvent was evaporated under reduced pressure to obtain the crude product. The crude product was further separated by silica gel column chromatography (petroleum ether/ethyl acetate=1:1 to 1:2) to give the title compound as a white solid, 0.06 g, 12.6% yield. MS (m/z): [M+H]⁺ calcd for C₂₅H₂₆N₆O₄, 475.52; found, 475.2. ¹H NMR (400 MHz, DMSO-d₆) δ 8.02 (d, J=6.8 Hz, 1H), 7.69-7.57 (m, 2H), 7.58 (d, J=4.1 Hz, 1H), 7.43-7.36 (m, 1H), 7.29 (d, J=8.1 Hz, 1H), 6.98 (t, J=5.0 Hz, 1H), 4.84 (s, 1H), 4.19-3.87 (m, 3H), 3.84-3.62 (m, 2H), 3.44-3.29 (m, 1H), 3.28 (s, 3H), 2.37 (dq, J=12.0, 6.7, 5.6 Hz, 1H), 1.92 (d, J=1.7 Hz, 3H), 1.88-1.66 (m, 1H), 1.58 (p, J=8.0, 7.0 Hz, 1H), 1.01 (d, J=7.1 Hz, 3H).

### Example 16

### tert-butyl ((S)-2-(4-(((3R, 4R)-1-(2-cyanoacetyl)-4-methylpiperidin-3-yl) (methyl) amino)-7H-pyrrolo [2, 3-d] pyrimidin-7-yl)-2-oxo-1-phenylethyl) carbamate

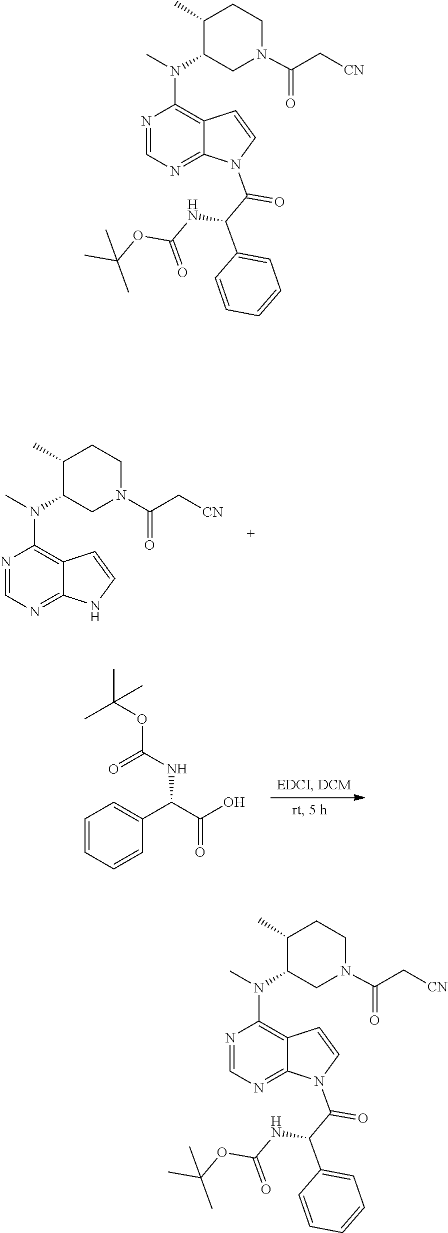 [SMILES: C[C@@H]1CCN(C(=O)CC#N)C[C@@H]1N(C)c1ncnc2c1ccn2C(=O)[C@@H](NC(=O)OC(C)(C)C)c1ccccc1 ; C[C@@H]1CCN(C(=O)CC#N)C[C@@H]1N(C)c1ncnc2[nH]ccc12 ; CC(C)(C)OC(=O)N[C@H](C(=O)O)c1ccccc1 ; C[C@@H]1CCN(C(=O)CC#N)C[C@@H]1N(C)c1ncnc2c1ccn2C(=O)[C@@H](NC(=O)OC(C)(C)C)c1ccccc1]

### Synthesis of tert-butyl ((S)-2-(4-(((3R, 4R)-1-(2-cyanoacetyl)-4-methylpiperidin-3-yl) (methyl) amino)-7H-pyrrolo [2, 3-d] pyrimidin-7-yl)-2-oxo-1-phenylethyl) carbamate

3-{(3R, 4R)-4-methyl-3-[methyl-(7H-pyrrolo [2, 3-d] pyrimidin-4-yl)-piperidin-1-yl]-3-oxo-propionitrile (tofacitinib, 312 mg, 1 mmol), N-Boc-L-phenylglycine (326 mg, 1.3 mmol) and 1-(3-dimethylaminopropyl)-3-ethylcarbodiimide hydrochloride (EDCI, 288 mg, 1.5 mmol) were dissolved in dichloromethane (10 mL) and stirred at room temperature for 5 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=1:1 to 1:2) to give the title compound as a white solid, 0.3 g, yield 55%. MS (m/z): [M+H]⁺ calcd for C₂₉H₃₅N₇O₄, 546.64; found, 546.3.1H NMR (400 MHz, DMSO-d6) δ 8.38 (d, J=4.7 Hz, 1H), 7.85 (d, J=7.3 Hz, 1H), 7.70 (d, J=4.1 Hz, 1H), 7.48 (t, J=7.4 Hz, 3H), 7.35-7.21 (m, 3H), 7.02-6.83 (m, 1H), 4.86 (s, 1H), 4.08-3.97 (m, 3H), 3.82-3.54 (m, 2H), 3.42-3.39 (m, 1H), 3.24 (s, 3H), 2.35 (s, 1H), 1.81-1.56 (m, 2H), 1.39 (s, 9H), 1.06-0.90 (m, 3H).

### Example 17

### (S)-2-(1-(ethylsulfonyl)-3-(4-(7-(2-(4-isobutylphenyl) propanoyl)-7H-pyrrolo [2, 3-d] pyrimidin-4-yl)-1H-pyrazol-1-yl) azetidin-3-yl) acetonitrile

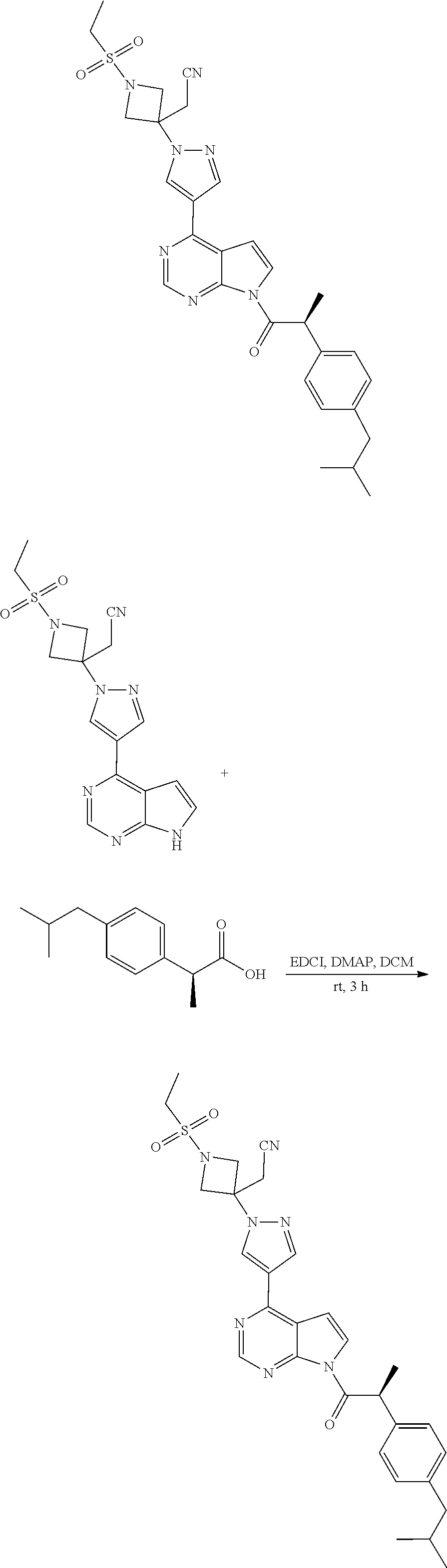

### Synthesis of (S)-2-(1-(ethylsulfonyl)-3-(4-(7-(2-(4-isobutylphenyl) propanoyl)-7H-pyrrolo [2, 3-d] pyrimidin-4-yl)-1H-pyrazol-1-yl) azetidin-3-yl) acetonitrile

1-(Ethylsulfonyl)-3-[4-(7H-pyrrolo [2, 3-d] pyrimidin-4-yl)-1H-pyrazol-1-yl]-3-azetidine acetonitrile (baricitinib, 742 mg, 2 mmol), 4-dimethylamino pyridine (DMAP, 488 mg, 4 mmol), (S)-(+)-2-(4-isobutylphenyl) propanoic acid ((S)-(+)-ibuprofen, 453 mg, 2.2 mmol) and 1-(3-dimethylaminopropyl)-3-ethylcarbodiimide hydrochloride (EDCI, 576 mg, 3 mmol) was dissolved in dichloromethane (20 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:2) to give the title compound as a white solid, 0.5 g, 44.7% yield. MS (m/z): [M+H]⁺ calcd for C₂₉H₃₃N₇O₃S, 560.69; found, 560.2.1H NMR (400 MHz, Chloroform-d) δ 8.96 (s, 1H), 8.41 (s, 1H), 8.26 (s, 1H), 8.03 (d, J=4.2 Hz, 1H), 7.47-7.34 (m, 2H), 7.06 (d, J=7.9 Hz, 2H), 6.81 (d, J=4.1 Hz, 1H), 6.06 (q, J=6.9 Hz, 1H), 4.62 (d, J=9.2 Hz, 2H), 4.34-4.16 (m, 2H), 3.40 (s, 2H), 3.08 (q, J=7.4 Hz, 2H), 2.39 (d, J=7.2 Hz, 2H), 1.80 (dp, J=13.5, 6.8 Hz, 1H), 1.68 (d, J=6.9 Hz, 3H), 1.41 (t, J=7.4 Hz, 3H), 0.95-0.82 (m, 6H).

### Example 18

### (S)-2-(1-(ethylsulfonyl)-3-(4-(7-(2-(6-methoxynaphthalen-2-yl) propanoyl)-7H-pyrrolo [2, 3-d] pyrimidin-4-yl)-1H-pyrazol-1-yl) azetidin-3-yl) acetonitrile

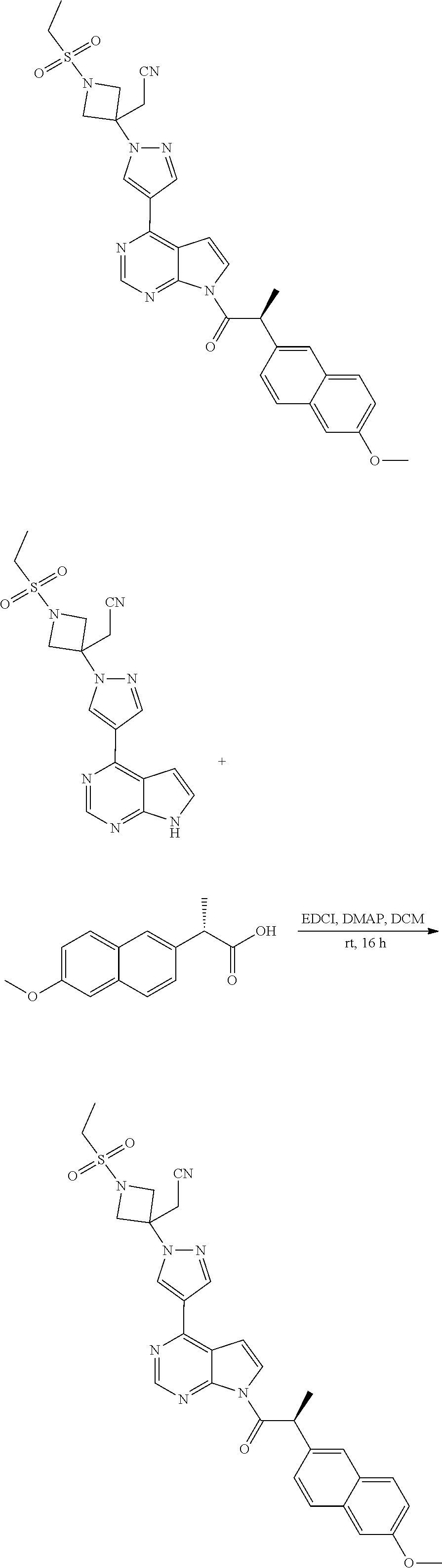

### Synthesis of (S)-2-(1-(ethylsulfonyl)-3-(4-(7-(2-(6-methoxynaphthalen-2-yl) propanoyl)-7H-pyrrolo [2, 3-d] pyrimidin-4-yl)-1H-pyrazol-1-yl) azetidin-3-yl) acetonitrile

1-(Ethylsulfonyl)-3-[4-(7H-pyrrolo [2, 3-d] pyrimidin-4-yl)-1H-pyrazol-1-yl]-3-azetidine acetonitrile (baricitinib, 371 mg, 1 mmol), 4-dimethylamino pyridine (DMAP, 305 mg, 2.5 mmol), (S)-(+)-2-(6-methoxy-2-naphthyl) propanoic acid (Naproxen, 230 mg, 1.1 mmol) and 1-(3-dimethylaminopropyl)-3-ethylcarbodiimide hydrochloride (EDCI, 288 mg, 1.5 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=1:1 to 1:2) to give the title compound as a white solid, 0.25 g, 42.9% yield. MS (m/z): [M+H]⁺ calcd for C₃₀H₂₉N₇O₄S, 584.67; found, 584.2.1H NMR (400 MHz, DMSO) δ 9.03 (s, 1H), 8.98 (s, 1H), 8.49 (s, 1H), 8.18 (d, J=4.2 Hz, 1H), 7.85 (s, 1H), 7.80-7.74 (m, 2H), 7.58-7.54 (m, 1H), 7.40 (d, J=4.2 Hz, 1H), 7.25 (d, J=2.2 Hz, 1H), 7.12 (dd, J=9.0, 2.4 Hz, 1H), 6.12 (q, J=6.8 Hz, 1H), 4.58 (d, J=9.2 Hz, 2H), 4.23 (d, J=9.2 Hz, 2H), 3.83 (s, 3H), 3.68 (s, 2H), 3.23 (q, J=7.3 Hz, 2H), 1.68 (d, J=6.9 Hz, 3H), 1.24 (t, J=7.3 Hz, 3H).

### Example 19

### 2-(3-(4-(7-(2-(3-benzoylphenyl) propanoyl)-7H-pyrrolo [2, 3-d] pyrimidin-4-yl)-1H-pyrazol-1-yl)-1-(ethylsulfonyl) azetidin-3-yl) acetonitrile

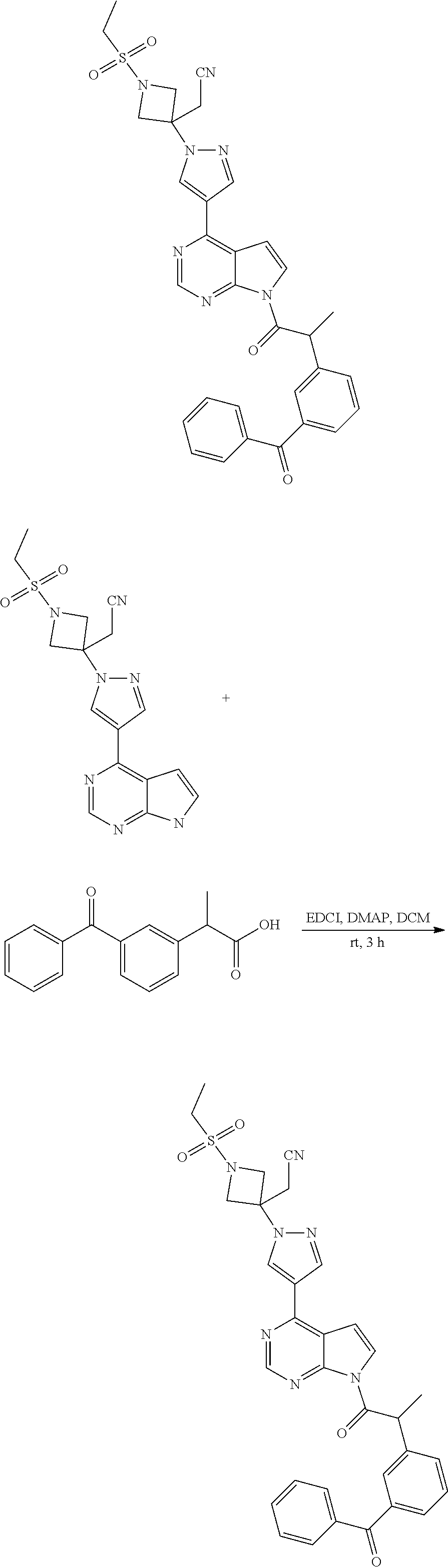 [SMILES: CCS(=O)(=O)N1CC(CC#N)(n2cc(-c3ncnc4c3ccn4C(=O)C(C)c3cccc(C(=O)c4ccccc4)c3)cn2)C1 ; CCS(=O)(=O)N1CC(CC#N)(n2cc(-c3ncnc4[nH]ccc34)cn2)C1 ; CC(C(=O)O)c1cccc(C(=O)c2ccccc2)c1 ; CCS(=O)(=O)N1CC(CC#N)(n2cc(-c3ncnc4c3ccn4C(=O)C(C)c3cccc(C(=O)c4ccccc4)c3)cn2)C1]

### Synthesis of 2-(3-(4-(7-(2-(3-benzoylphenyl) propanoyl)-7H-pyrrolo [2, 3-d] pyrimidin-4-yl)-1H-pyrazol-1-yl)-1-(ethylsulfonyl) azetidin-3-yl) acetonitrile

1-(Ethylsulfonyl)-3-[4-(7H-pyrrolo [2, 3-d] pyrimidin-4-yl)-1H-pyrazol-1-yl]-3-azetidine acetonitrile (baricitinib, 371 mg, 1 mmol), 4-dimethylamino pyridine (DMAP, 244 mg, 2 mmol), 2-(3-benzoylphenyl) propanoic acid (ketoprofen, 280 mg, 1.1 mmol) and 1-(3-dimethylaminopropyl)-3-ethylcarbodiimide hydrochloride (EDCI, 288 mg, 1.5 mmol) were dissolved in dichloromethane (15 mL) and stirred at room temperature for 3 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:2) to give the title compound as a white solid, 0.2 g, 32.9% yield. MS (m/z): [M+H]⁺ calcd for C₃₂H₂₉N₇O₄S, 608.69; found, 608.3. ¹H NMR (400 MHz, DMSO) δ 9.00 (s, 1H), 8.93 (s, 1H), 8.51 (s, 1H), 8.17 (d, J=4.2 Hz, 1H), 7.81 (s, 1H), 7.76 (d, J=7.7 Hz, 1H), 7.70-7.58 (m, 4H), 7.52 (t, J=7.6 Hz, 3H), 7.43 (d, J=4.2 Hz, 1H), 6.06 (q, J=6.8 Hz, 1H), 4.59 (d, J=9.1 Hz, 2H), 4.24 (d, J=9.1 Hz, 2H), 3.68 (s, 2H), 3.23 (q, J=7.3 Hz, 2H), 1.63 (d, J=6.9 Hz, 3H), 1.24 (t, J=7.3 Hz, 3H).

### Example 20

### 2-(1-(ethylsulfonyl)-3-(4-(7-(2-(2-fluoro-[1, 1′-biphenyl]-4-yl) propanoyl)-7H-pyrrolo [2, 3-d] pyrimidin-4-yl)-1H-pyrazol-1-yl) azetidin-3-yl) acetonitrile

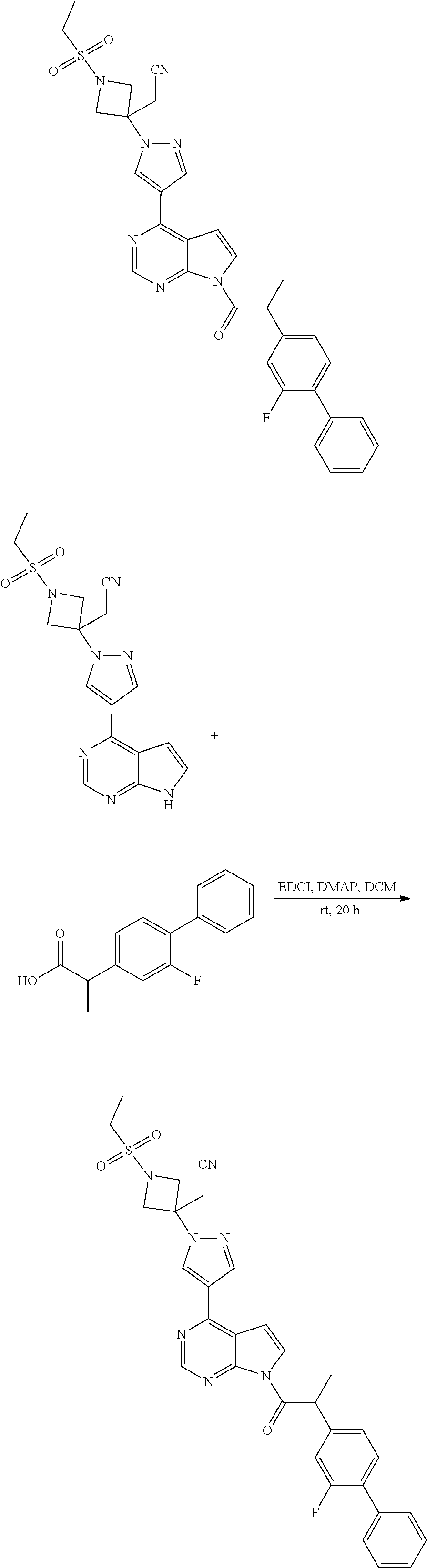

### Synthesis of 2-(1-(ethylsulfonyl)-3-(4-(7-(2-(2-fluoro-[1, 1′-biphenyl]-4-yl) propanoyl)-7H-pyrrolo [2, 3-d] pyrimidin-4-yl)-1H-pyrazol-1-yl) azetidin-3-yl) acetonitrile

1-(Ethylsulfonyl)-3-[4-(7H-pyrrolo [2, 3-d] pyrimidin-4-yl)-1H-pyrazol-1-yl]-3-azetidine acetonitrile (baricitinib, 742 mg, 2 mmol), 4-dimethylamino pyridine (DMAP, 610 mg, 5 mmol), 2-(2-fluoro-4-biphenyl) propanoic acid (flurbiprofen, 537 mg, 2.2 mmol) and 1-(3-dimethylaminopropyl)-3-ethylcarbodiimide hydrochloride (EDCI, 576 mg, 3 mmol) were dissolved in dichloromethane (20 mL) and stirred at room temperature for 20 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=1:1 to 1:2) to give the title compound as a white solid, 0.3 g, 25.1% yield. MS (m/z): [M+H]⁺ calcd for C₃₁H₂₈ FN₇O₃S, 598.67; found, 598.2.1H NMR (400 MHz, DMSO) δ 9.05 (s, 1H), 9.02 (s, 1H), 8.53 (s, 1H), 8.20 (d, J=4.2 Hz, 1H), 7.54-7.35 (m, 9H), 6.08 (q, J=7.0 Hz, 1H), 4.61 (d, J=9.2 Hz, 2H), 4.25 (d, J=9.2 Hz, 2H), 3.70 (s, 2H), 3.24 (q, J=7.4 Hz, 2H), 1.66 (d, J=7.0 Hz, 3H), 1.25 (t, J=7.3 Hz, 3H).

### Example 21

### 2-(1-(Ethylsulfonyl)-3-(4-(7-(2-(3-phenoxyphenyl) propanoyl)-7H-pyrrolo [2, 3-d] pyrimidin-4-yl)-1H-pyrazol-1-yl) azetidin-3-yl) acetonitrile

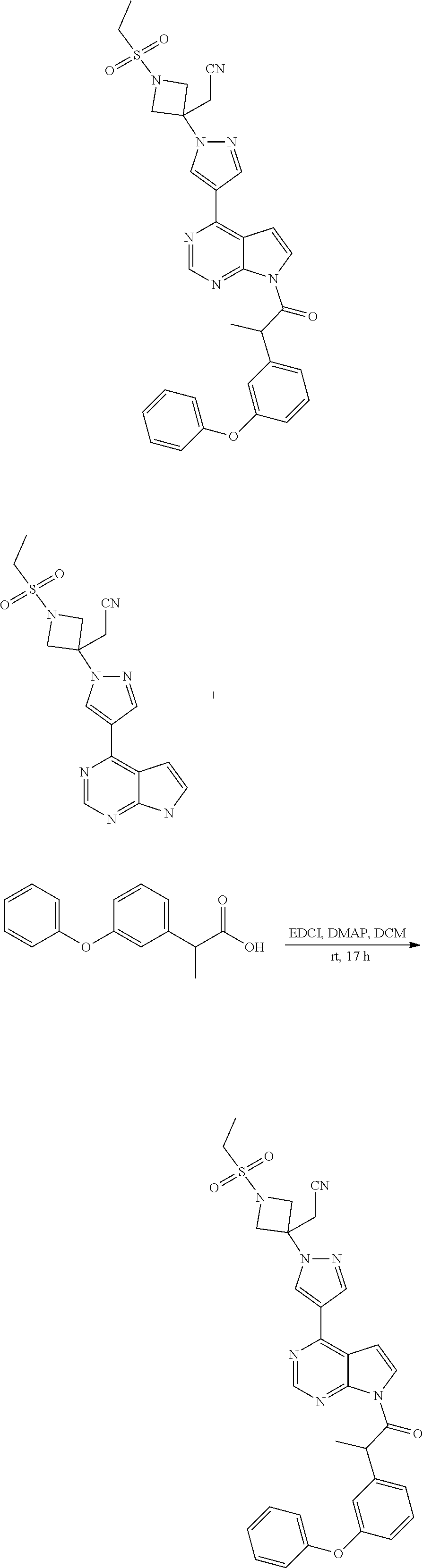 [SMILES: CCS(=O)(=O)N1CC(CC#N)(n2cc(-c3ncnc4c3ccn4C(=O)C(C)c3cccc(Oc4ccccc4)c3)cn2)C1 ; CCS(=O)(=O)N1CC(CC#N)(n2cc(-c3ncnc4[nH]ccc34)cn2)C1 ; CC(C(=O)O)c1cccc(Oc2ccccc2)c1 ; CCS(=O)(=O)N1CC(CC#N)(n2cc(-c3ncnc4c3ccn4C(=O)C(C)c3cccc(Oc4ccccc4)c3)cn2)C1]

### Synthesis of 2-(1-(ethylsulfonyl)-3-(4-(7-(2-(3-phenoxyphenyl) propanoyl)-7H-pyrrolo [2, 3-d] pyrimidin-4-yl)-1H-pyrazol-1-yl) azetidin-3-yl) acetonitrile

1-(Ethylsulfonyl)-3-[4-(7H-pyrrolo [2, 3-d] pyrimidin-4-yl)-1H-pyrazol-1-yl]-3-azetidine acetonitrile (baricitinib, 186 mg, 0.5 mmol), 4-dimethylamino pyridine (DMAP, 61 mg, 0.5 mmol), 2-(3-phenoxyphenyl) propanoic acid (fenoprofen, 133 mg, 0.55 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 17 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:2) to give the title compound as a white solid, 0.11 g, 74.5% yield. MS (m/z): [M+H]⁺ calcd for C₃₁H₂₉N₇O₄S, 596.68; found, 596.2. ¹H NMR (400 MHz, DMSO-d₆) δ 8.99 (s, 1H), 8.86 (s, 1H), 8.50 (s, 1H), 8.12 (d, J=4.2 Hz, 1H), 7.40 (d, J=4.2 Hz, 1H), 7.37-7.25 (m, 3H), 7.21-7.10 (m, 2H), 7.07 (t, J=2.1 Hz, 1H), 6.95-6.89 (m, 2H), 6.86-6.78 (m, 1H), 5.94 (d, J=6.9 Hz, 1H), 4.60 (d, J=9.1 Hz, 2H), 4.24 (d, J=9.1 Hz, 2H), 3.68 (s, 2H), 3.23 (q, J=7.3 Hz, 2H), 1.57 (d, J=6.9 Hz, 3H), 1.24 (t, J=7.4 Hz, 3H).

### Example 22

### 2-(1-(ethylsulfonyl)-3-(4-(7-(2-(4-(2-oxocyclopentyl) methyl) phenyl) propanoyl)-7H-pyrrolo [2, 3-d] pyrimidin-4-yl)-1H-pyrazol-1-yl) azetidin-3-yl) acetonitrile

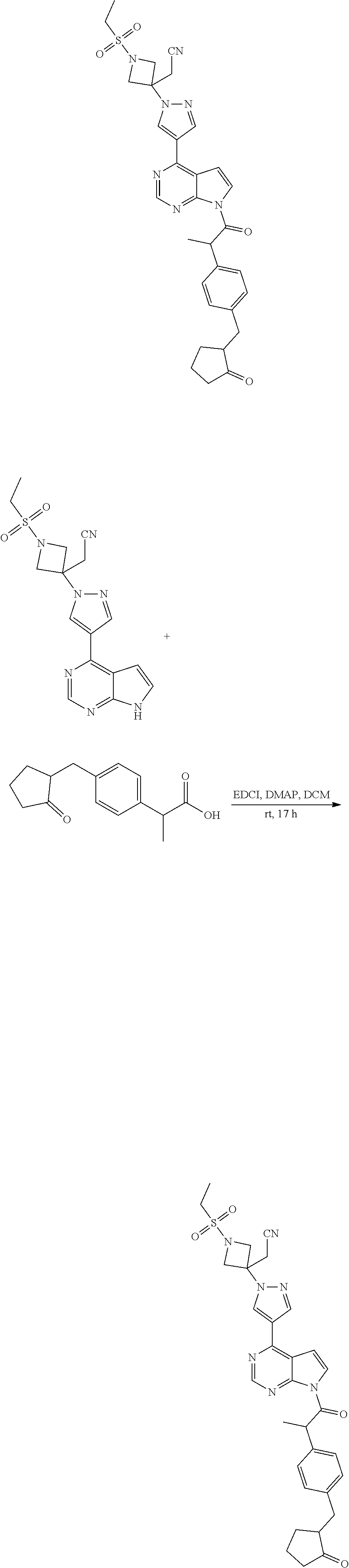 [SMILES: CCS(=O)(=O)N1CC(CC#N)(n2cc(-c3ncnc4c3ccn4C(=O)C(C)c3ccc(CC4CCCC4=O)cc3)cn2)C1 ; CCS(=O)(=O)N1CC(CC#N)(n2cc(-c3ncnc4[nH]ccc34)cn2)C1 ; CC(C(=O)O)c1ccc(CC2CCCC2=O)cc1 ; CCS(=O)(=O)N1CC(CC#N)(n2cc(-c3ncnc4c3ccn4C(=O)C(C)c3ccc(CC4CCCC4=O)cc3)cn2)C1]

### Synthesis of 2-(1-(ethylsulfonyl)-3-(4-(7-(2-(4-(2-oxocyclopentyl) methyl) phenyl) propanoyl)-7H-pyrrolo [2, 3-d] pyrimidin-4-yl)-1H-pyrazol-1-yl) azetidin-3-yl) acetonitrile

1-(Ethylsulfonyl)-3-[4-(7H-pyrrolo [2, 3-d] pyrimidin-4-yl)-1H-pyrazol-1-yl]-3-azetidine acetonitrile (baricitinib, 186 mg, 0.5 mmol), 4-dimethylamino pyridine (DMAP, 61 mg, 0.5 mmol), 2-[4-(2-oxocyclopentan-1-ylmethyl) phenyl] propanoic acid (loxoprofen, 136 mg, 0.55 mmol) and 1-(3-dimethylaminopropyl)-3-ethylcarbodiimide hydrochloride (EDCI, 144 mg, 0.75 mmol) was dissolved in dichloromethane (10 mL) and stirred at room temperature for 17 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 3:4) to give the title compound as a white solid, 0.13 g, 43.4% yield. MS (m/z): [M+H]⁺ calcd for C₃₁H₃₃N₇O₄S, 600.71; found, 600.3. ¹H NMR (400 MHz, DMSO-d₆) δ 8.99 (d, J=3.4 Hz, 2H), 8.50 (s, 1H), 8.14 (d, J=4.2 Hz, 1H), 7.40 (d, J=4.2 Hz, 1H), 7.34 (d, J=7.9 Hz, 2H), 7.12 (d, J=7.9 Hz, 2H), 5.98 (q, J=6.9 Hz, 1H), 4.59 (d, J=9.1 Hz, 2H), 4.24 (d, J=9.1 Hz, 2H), 3.69 (s, 2H), 3.33-3.19 (m, 2H), 2.96-2.83 (m, 1H), 2.44-2.27 (m, 2H), 2.20 (dd, J=18.5, 8.5 Hz, 1H), 2.03 (ddd, J=18.6, 10.1, 8.6 Hz, 1H), 1.95-1.74 (m, 2H), 1.70-1.61 (m, 1H), 1.58 (d, J=6.9 Hz, 3H), 1.42 (dd, J=10.6, 6.9 Hz, 1H), 1.24 (dd, J=8.7, 6.1 Hz, 3H).

### Example 23

### 2-(3-(4-(7-(2-(1-(4-chlorobenzoyl)-5-methoxy-2-methyl-1H-indol-3-yl) acetyl)-7H-pyrrolo [2, 3-d] pyrimidin-4-yl)-1H-pyrazol-1-yl)-1-(ethylsulfonyl) azetidin-3-yl) acetonitrile

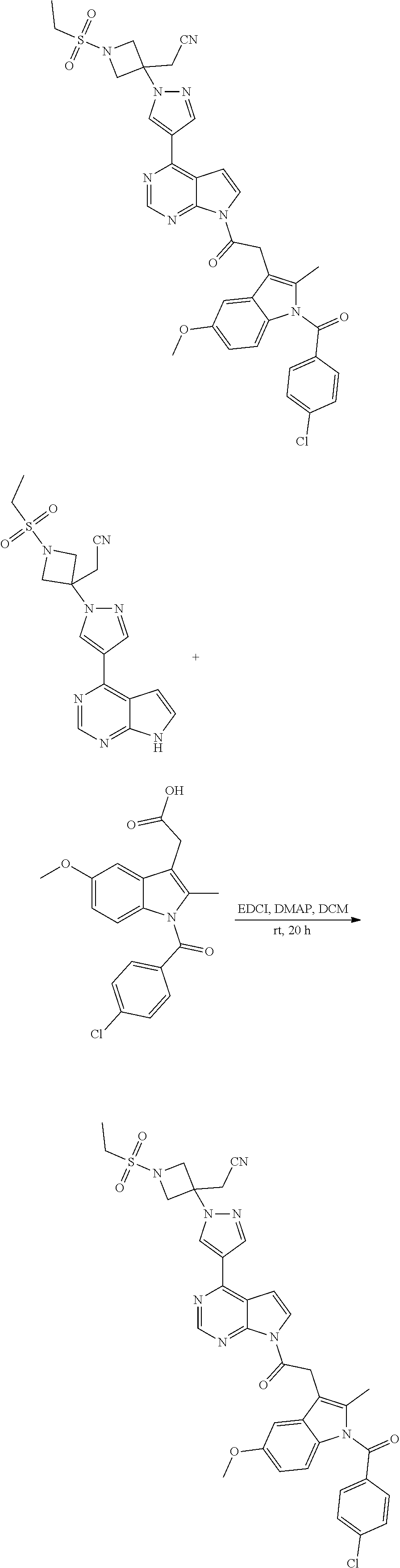

### Synthesis of 2-(3-(4-(7-(2-(1-(4-chlorobenzoyl)-5-methoxy-2-methyl-1H-indol-3-yl) acetyl)-7H-pyrrolo [2, 3-d] pyrimidin-4-yl)-1H-pyrazol-1-yl)-1-(ethylsulfonyl) azetidin-3-yl) acetonitrile

1-(Ethylsulfonyl)-3-[4-(7H-pyrrolo [2, 3-d] pyrimidin-4-yl)-1H-pyrazol-1-yl]-3-azetidine acetonitrile (baricitinib, 186 mg, 0.5 mmol), 4-dimethylamino pyridine (DMAP, 61 mg, 0.5 mmol), 1-(4-chlorobenzoyl)-5-methoxy-2-methyl-1H-indole-3-acetic acid (indomethacin, 197 mg, 0.55 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 20 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=1:1 to 1:5) to give the title compound as a white solid, 0.18 g, 50.6% yield. MS (m/z): [M+H]⁺ calcd for C₃₅H₃₁ClN₈O₅S, 712.19; found, 711.2. ¹H NMR (400 MHz, DMSO-d₆) δ 9.05 (d, J=2.4 Hz, 2H), 8.56 (s, 1H), 8.17 (d, J=4.2 Hz, 1H), 7.77-7.61 (m, 4H), 7.47 (d, J=4.2 Hz, 1H), 7.18 (d, J=2.6 Hz, 1H), 6.99 (d, J=9.0 Hz, 1H), 6.72 (dd, J=9.0, 2.6 Hz, 1H), 5.04 (s, 2H), 4.64 (d, J=9.1 Hz, 2H), 4.28 (d, J=9.1 Hz, 2H), 3.71 (d, J=6.8 Hz, 5H), 3.25 (q, J=7.3 Hz, 2H), 2.27 (s, 3H), 1.26 (t, J=7.3 Hz, 3H).

### Example 24

### N-(4-(2-(4-(1-(3-(cyanomethyl)-1-(ethylsulfonyl) azetidin-3-yl)-1H-pyrazol-4-yl)-7H-pyrrolo [2, 3-d] pyrimidin-7-yl)-2-oxoethyl) phenyl) acetamide

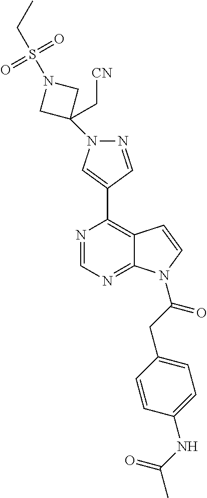 [SMILES: CCS(=O)(=O)N1CC(CC#N)(n2cc(-c3ncnc4c3ccn4C(=O)Cc3ccc(NC(C)=O)cc3)cn2)C1]

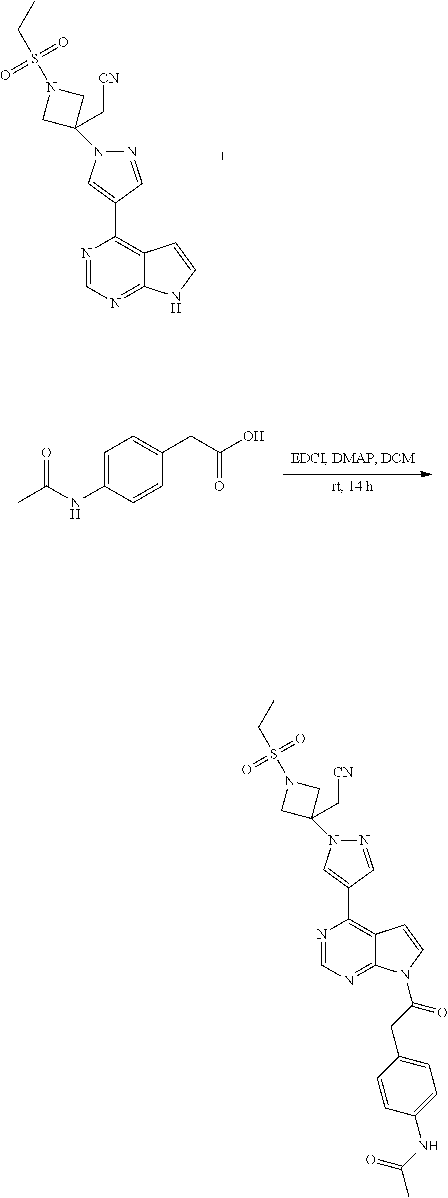 [SMILES: CCS(=O)(=O)N1CC(CC#N)(n2cc(-c3ncnc4[nH]ccc34)cn2)C1 ; CC(=O)Nc1ccc(CC(=O)O)cc1 ; "rt, 14 h" ; CCS(=O)(=O)N1CC(CC#N)(n2cc(-c3ncnc4c3ccn4C(=O)Cc3ccc(NC(C)=O)cc3)cn2)C1]

### Synthesis of N-(4-(2-(4-(1-(3-(cyanomethyl)-1-(ethylsulfonyl) azetidin-3-yl)-1H-pyrazol-4-yl)-7H-pyrrolo [2, 3-d] pyrimidin-7-yl)-2-oxoethyl) phenyl) acetamide

1-(Ethylsulfonyl)-3-[4-(7H-pyrrolo [2, 3-d] pyrimidin-4-yl)-1H-pyrazol-1-yl]-3-azetidine acetonitrile (baricitinib, 186 mg, 0.5 mmol), 4-dimethylamino pyridine (DMAP, 61 mg, 0.5 mmol), 2-(4-acetamidophenyl) acetic acid (actarit, 106 mg, 0.55 mmol) and 1-(3-dimethylaminopropyl)-3-ethylcarbodiimide hydrochloride (EDCI, 144 mg, 0.75 mmol) was dissolved in a mixed solvent of dichloromethane (10 mL) and DMF (2 mL) and stirred at room temperature for 14 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:2) to give the title compound as a white solid, 0.03 g, 10% yield. MS (m/z): [M+H]⁺ calcd for C₂₆H₂₆N₈O₄S, 547.61; found, 547.3. ¹H NMR (400 MHz, DMSO-d₆) δ 9.93 (s, 1H), 9.01 (d, J=10.0 Hz, 2H), 8.53 (s, 1H), 8.14 (d, J=4.2 Hz, 1H), 7.54 (d, J=8.1 Hz, 2H), 7.44 (d, J=4.2 Hz, 1H), 7.30 (d, J=8.0 Hz, 2H), 4.87 (s, 2H), 4.62 (d, J=9.0 Hz, 2H), 4.26 (d, J=9.1 Hz, 2H), 3.71 (s, 2H), 3.25 (q, J=7.3 Hz, 2H), 2.04 (s, 3H), 1.26 (t, J=7.4 Hz, 3H).

### Example 25

### 2-(3-(4-(7-(2-(1, 8-diethyl-1, 3, 4, 9-tetrahydropyrano [3, 4-b] indol-1-yl) acetyl)-7H-pyrrolo [2, 3-d] pyrimidin-4-yl)-1H-pyrazol-1-yl)-1-(ethylsulfonyl) azetidin-3-yl) acetonitrile

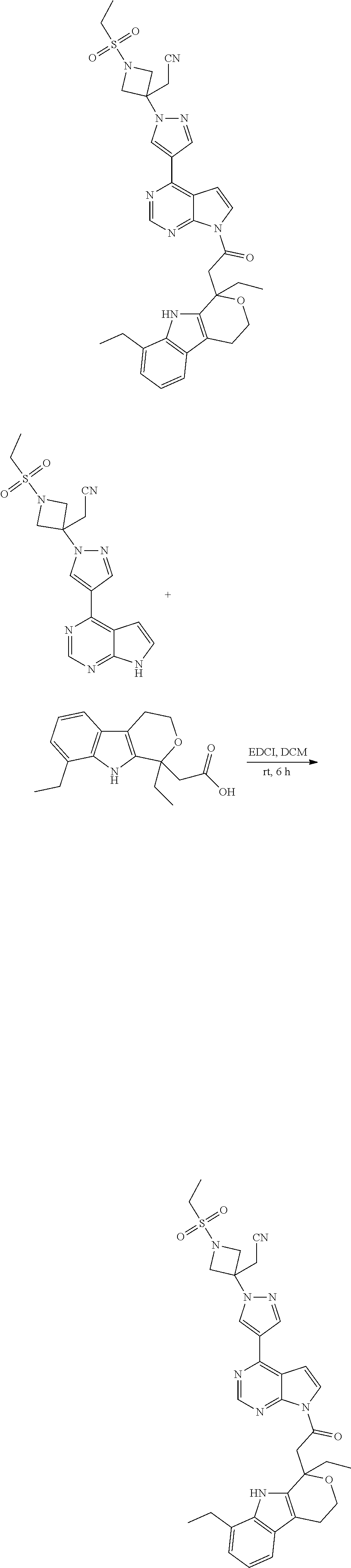 [SMILES: CCc1cccc2c3c([nH]c12)C(CC)(CC(=O)n1ccc2c(-c4cnn(C5(CC#N)CN(S(=O)(=O)CC)C5)c4)ncnc21)OCC3 ; CCS(=O)(=O)N1CC(CC#N)(n2cc(-c3ncnc4[nH]ccc34)cn2)C1 ; CCc1cccc2c3c([nH]c12)C(CC)(CC(=O)O)OCC3 ; CCc1cccc2c3c([nH]c12)C(CC)(CC(=O)n1ccc2c(-c4cnn(C5(CC#N)CN(S(=O)(=O)CC)C5)c4)ncnc21)OCC3]

### Synthesis of 2-(3-(4-(7-(2-(1, 8-diethyl-1, 3, 4, 9-tetrahydropyrano [3, 4-b] indol-1-yl) acetyl)-7H-pyrrolo [2, 3-d] pyrimidin-4-yl)-1H-pyrazol-1-yl)-1-(ethylsulfonyl) azetidin-3-yl) acetonitrile

1-(Ethylsulfonyl)-3-[4-(7H-pyrrolo [2, 3-d] pyrimidin-4-yl)-1H-pyrazol-1-yl]-3-azetidine acetonitrile (baricitinib, 186 mg, 0.5 mmol), 4-dimethylamino pyridine (DMAP, 61 mg, 0.5 mmol), 1, 8-diethyl-1, 3, 4, 9-tetrahydropyran [3, 4-b] indole-1-acetic acid (etodolac, 158 mg, 0.55 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 14 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:1) to give the title compound as a white solid, 0.18 g, 56% yield. MS (m/z): [M+H]⁺ calcd for C₃₃H₃₆N₈O₄S, 641.76; found, 641.2. ¹H NMR (400 MHz, DMSO-d₆) δ 10.59 (s, 1H), 9.04 (s, 1H), 8.95 (s, 1H), 8.55 (s, 1H), 8.15 (d, J=4.2 Hz, 1H), 7.44 (d, J=4.2 Hz, 1H), 7.27 (dd, J=7.4, 1.6 Hz, 1H), 7.00-6.88 (m, 2H), 4.71-4.56 (m, 3H), 4.29 (d, J=9.1 Hz, 2H), 4.22 (d, J=14.5 Hz, 1H), 3.91 (ddd, J=11.7, 7.1, 4.9 Hz, 1H), 3.83-3.76 (m, 1H), 3.74 (s, 2H), 3.27 (q, J=7.3 Hz, 2H), 2.89 (q, J=7.5 Hz, 2H), 2.64 (dt, J=6.8, 4.2 Hz, 2H), 2.21 (q, J=7.2 Hz, 2H), 1.29 (td, J=7.4, 4.3 Hz, 6H), 0.76 (t, J=7.3 Hz, 3H).

### Example 26

### Tert-butyl (S)-(2-(4-(1-(3-(cyanomethyl)-1-(ethylsulfonyl) azetidin-3-yl)-1H-pyrazol-4-yl)-7H-pyrrolo [2, 3-d] pyrimidin-7-yl)-2-oxo-1-phenylethyl) carbamate

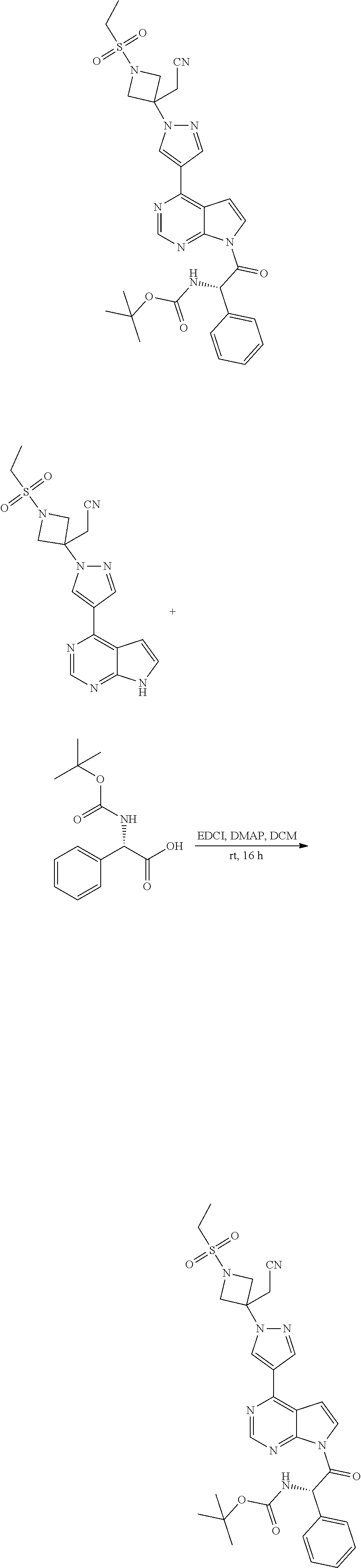 [SMILES: CCS(=O)(=O)N1CC(CC#N)(n2cc(-c3ncnc4c3ccn4C(=O)[C@@H](NC(=O)OC(C)(C)C)c3ccccc3)cn2)C1 ; CCS(=O)(=O)N1CC(CC#N)(n2cc(-c3ncnc4[nH]ccc34)cn2)C1 ; CC(C)(C)OC(=O)N[C@H](C(=O)O)c1ccccc1 ; CCS(=O)(=O)N1CC(CC#N)(n2cc(-c3ncnc4c3ccn4C(=O)[C@@H](NC(=O)OC(C)(C)C)c3ccccc3)cn2)C1]

Synthesis of tert-butyl (S)-(2-(4-(1-(3-(cyanomethyl)-1-(ethylsulfonyl) azetidin-3-yl)-1H-pyrazol-4-yl)-7H-pyrrolo [2, 3-d] pyrimidin-7-yl)-2-oxo-1-phenylethyl) carbamate 1-(Ethylsulfonyl)-3-[4-(7H-pyrrolo [2, 3-d] pyrimidin-4-yl)-1H-pyrazol-1-yl]-3-azetidine acetonitrile (baricitinib, 371 mg, 1 mmol), N-Boc-L-phenylglycine (301.5 mg, 1.2 mmol), 4-dimethylamino pyridine (DMAP, 183 mg, 1.5 mmol) and 1-(3-dimethylaminopropyl)-3-ethylcarbodiimide hydrochloride (EDCI, 288 mg, 1.5 mmol) were dissolved in dichloromethane (15 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:2) to give the title compound as a white solid, 0.12 g, 19.8% yield. MS (m/z): [M+H]⁺ calcd for C₂₉H₃₂N₈O₅S, 605.69; found, 605.3. ¹H NMR (400 MHz, DMSO-d₆) δ 9.00 (d, J=3.7 Hz, 2H), 8.51 (s, 1H), 8.18 (d, J=4.1 Hz, 1H), 7.99 (d, J=7.5 Hz, 1H), 7.61-7.37 (m, 4H), 7.36-7.23 (m, 3H), 4.60 (d, J=9.1 Hz, 2H), 4.24 (d, J=9.1 Hz, 2H), 3.69 (s, 2H), 3.23 (q, J=7.3 Hz, 2H), 1.40 (s, 9H), 1.24 (t, J=7.4 Hz, 3H).

### Example 27

### 1-((1S, 4R)-4-((7-((S)-2-(4-isobutylphenyl) propanoyl)-7H-pyrrolo [2, 3-d] pyrimidin-4-yl) (methyl) amino) cyclohexyl)-N-methyl methanesulfonamide

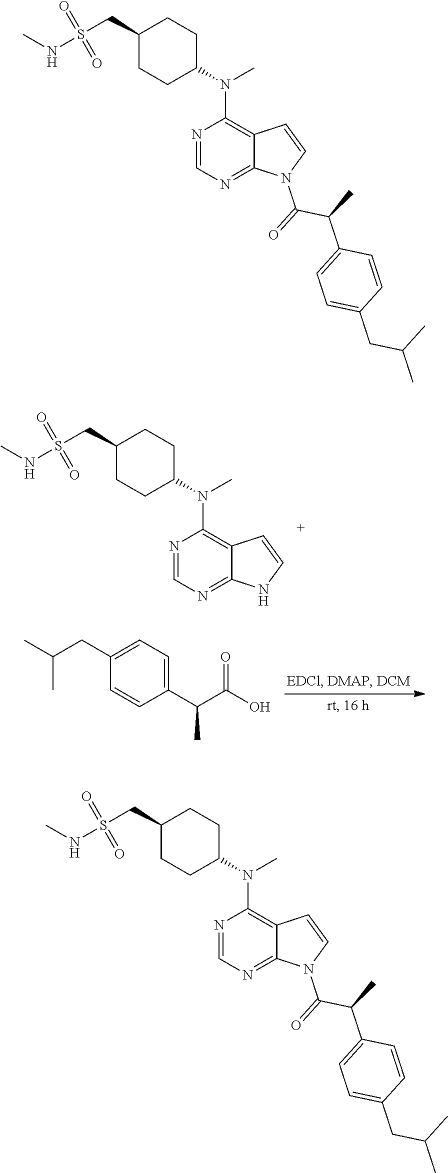

N-methyl-1-((1R, 4R)-4-(methyl (7H-pyrrolo [2, 3-d] pyrimidin-4-yl) amino) cyclohexyl) methanesulfonamide (oclacitinib, 338 mg, 1 mmol), 4-dimethylamino pyridine (DMAP, 122 mg, 1 mmol), (S)-(+)-2-(4-isobutylphenyl) propanoic acid ((S)-(+)-ibuprofen, 247 mg, 1.2 mmol) and 1-(3-dimethylaminopropyl)-3-ethylcarbodiimide hydrochloride (EDCI, 288 mg, 1.5 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 2:1) to give the title compound as a white solid, 0.14 g, 26.6% yield. MS (m/z): [M+H]⁺ calcd for C₂₈H₃₉N₅O₃S, 526.71; found, 526.2. ¹H NMR (400 MHz, DMSO-d₆) δ 8.36 (s, 1H), 7.66 (d, J=4.2 Hz, 1H), 7.29 (d, J=8.1 Hz, 2H), 7.06 (d, J=7.8 Hz, 2H), 6.93-6.79 (m, 2H), 6.10 (q, J=6.9 Hz, 1H), 4.63 (s, 1H), 3.14 (s, 3H), 2.94 (d, J=6.2 Hz, 2H), 2.59 (d, J=5.0 Hz, 3H), 2.35 (d, J=7.2 Hz, 2H), 2.04 (d, J=13.1 Hz, 2H), 1.88-1.74 (m, 2H), 1.74-1.63 (m, 4H), 1.52 (d, J=7.0 Hz, 3H), 1.27-1.21 (m, 2H), 0.81 (d, J=6.6 Hz, 6H).

### Example 28

### (R)-3-cyclopentyl-3-(4-(7-(S)-2-(4-isobutylphenyl) propanoyl)-7H-pyrrolo [2, 3-d] pyrimidin-4-yl)-1H-pyrazol-1-yl) propanenitrile

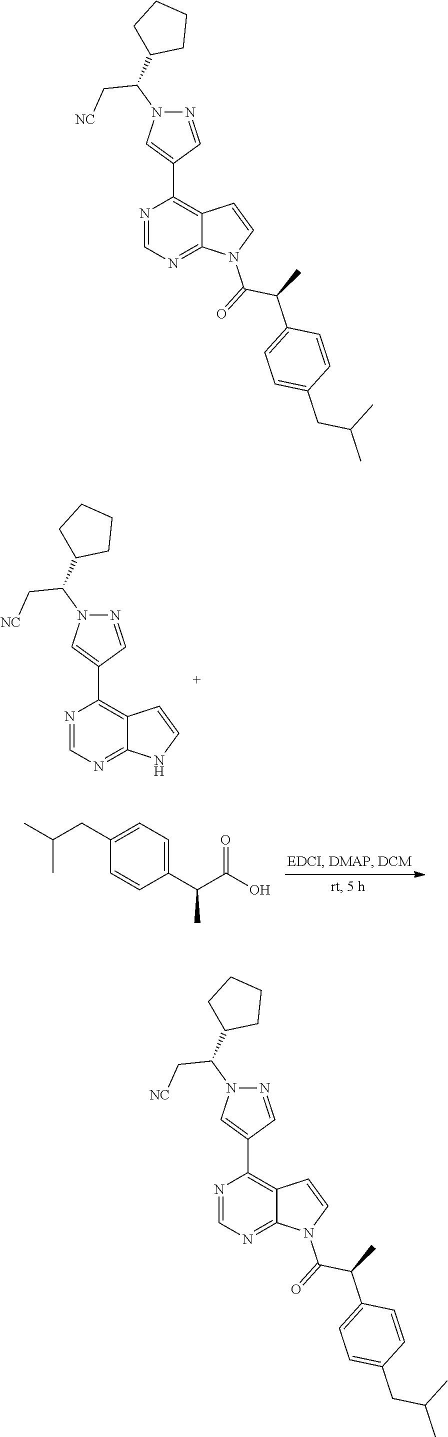

(R)-3-(4-(7h-pyrrolo [2, 3-d] pyridin-4-yl)-1h-pyrazol-1-yl)-3-cyclopentylpropane (ruxolitinib, 306 mg, 1 mmol), 4-dimethylamino pyridine (DMAP, 12 mg, 0.1 mmol), (S)-(+)-2-(4-isobutylphenyl) propanoic acid ((S)-(+)-ibuprofen, 247 mg, 1.2 mmol) and 1-(3-dimethylaminopropyl)-3-ethylcarbodiimide hydrochloride (EDCI, 288 mg, 1.5 mmol) were dissolved in dichloromethane (10 mL) and stirred at room temperature for 5 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 3:1) to give the title compound as a white solid, 0.42 g, 85% yield. MS (m/z): [M+H]⁺ calcd for C₃₀H₃₄N₆O₄, 495.64; found, 495.2.1H NMR (400 MHz, DMSO-d₆) δ 8.96 (s, 1H), 8.87 (s, 1H), 8.40 (s, 1H), 8.11 (d, J=4.2 Hz, 1H), 7.36-7.27 (m, 3H), 7.07 (d, J=8.0 Hz, 2H), 6.00 (q, J=6.9 Hz, 1H), 4.53 (td, J=9.6, 4.3 Hz, 1H), 3.23 (qd, J=17.1, 6.9 Hz, 2H), 2.47-2.38 (m, 1H), 2.34 (d, J=7.1 Hz, 2H), 1.77 (ddd, J=27.2, 12.6, 7.2 Hz, 2H), 1.66-1.47 (m, 5H), 1.47-1.11 (m, 5H), 0.88-0.76 (m, 6H).

### Example 29

### 3-((3R, 4R)-4-methyl-3-(methyl (7-(2-(4-(1-oxoisoindolin-2-yl) phenyl) butanoyl)-7H-pyrrolo [2, 3-d] pyrimidin-4-yl) amino) piperidin-1-yl)-3-oxopropanenitrile

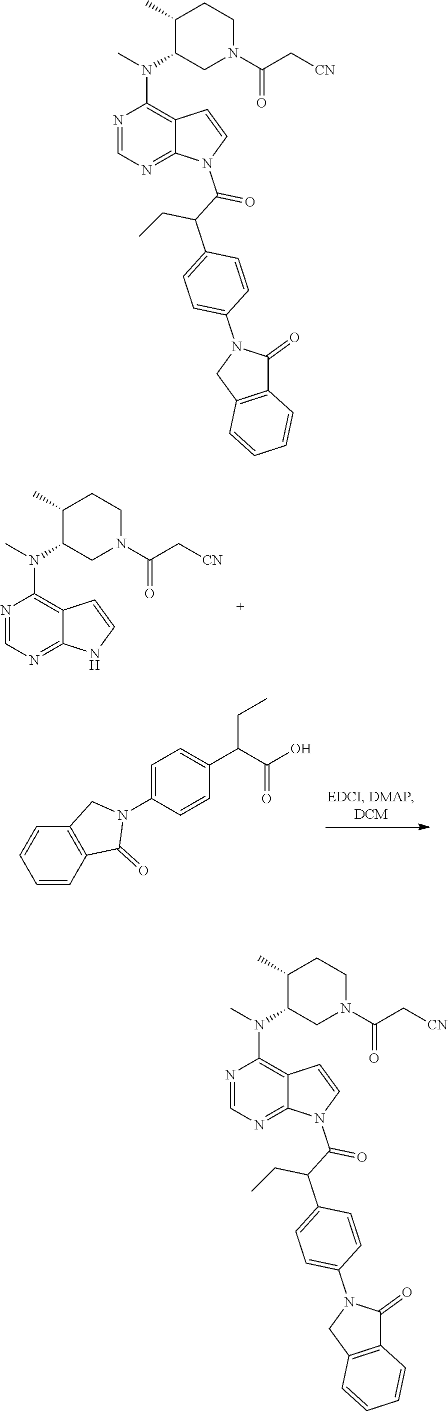 [SMILES: CCC(C(=O)n1ccc2c(N(C)[C@H]3CN(C(=O)CC#N)CC[C@H]3C)ncnc21)c1ccc(N2Cc3ccccc3C2=O)cc1 ; C[C@@H]1CCN(C(=O)CC#N)C[C@@H]1N(C)c1ncnc2[nH]ccc12 ; CCC(C(=O)O)c1ccc(N2Cc3ccccc3C2=O)cc1 ; CCC(C(=O)n1ccc2c(N(C)[C@H]3CN(C(=O)CC#N)CC[C@H]3C)ncnc21)c1ccc(N2Cc3ccccc3C2=O)cc1]

3-{(3R, 4R)-4-methyl-3-[methyl-(7H-pyrrolo [2, 3-d] pyrimidin-4-yl)-piperidin-1-yl]-3-oxo-propionitrile (tofacitinib, 312 mg, 1 mmol), 4-dimethylamino pyridine (DMAP, 12 mg, 0.1 mmol), 2-(4-(1-oxoisoindolin-2-yl) phenyl) butanoic acid (indobufen, 384 mg, 1.3 mmol) and 1-(3-dimethylaminopropyl)-3-ethylcarbodiimide hydrochloride (EDCI, 249 mg, 1.3 mmol) was dissolved in dichloromethane (10 mL) and stirred at room temperature for 20 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=1:1 to 1:5) to give the title compound as a white solid, 0.27 g, 45.8% yield. MS (m/z): [M+H]⁺ calcd for C₃₄H₃₅N₇O₃, 590.70; found, 590.2. ¹H NMR (400 MHz, DMSO-d₆) δ 8.52-8.37 (m, 1H), 7.84 (d, J=8.1 Hz, 2H), 7.74 (dd, J=22.3, 5.9 Hz, 2H), 7.66 (d, J=7.1 Hz, 2H), 7.57-7.50 (m, 1H), 7.50-7.44 (m, 2H), 6.90 (d, J=4.6 Hz, 1H), 6.01-5.91 (m, 1H), 4.97 (s, 2H), 4.84 (s, 1H), 4.18-4.00 (m, 2H), 3.99-3.53 (m, 3H), 3.41 (d, J=6.3 Hz, 1H), 3.24 (s, 3H), 2.43-2.30 (m, 1H), 2.27-2.14 (m, 1H), 1.95-1.50 (m, 3H), 1.00 (d, J=7.1 Hz, 3H), 0.91 (t, J=7.3 Hz, 3H).

### Example 30

### 2-2-(1-(Ethylsulfonyl)-3-(4-(7-(2-(4-(1-oxoisoindolin-2-yl) phenyl) butanoyl)-7H-pyrrolo [2, 3-d] pyrimidin-4-yl)-1H-pyrazol-1-yl) azetidin-3-yl) acetonitrile

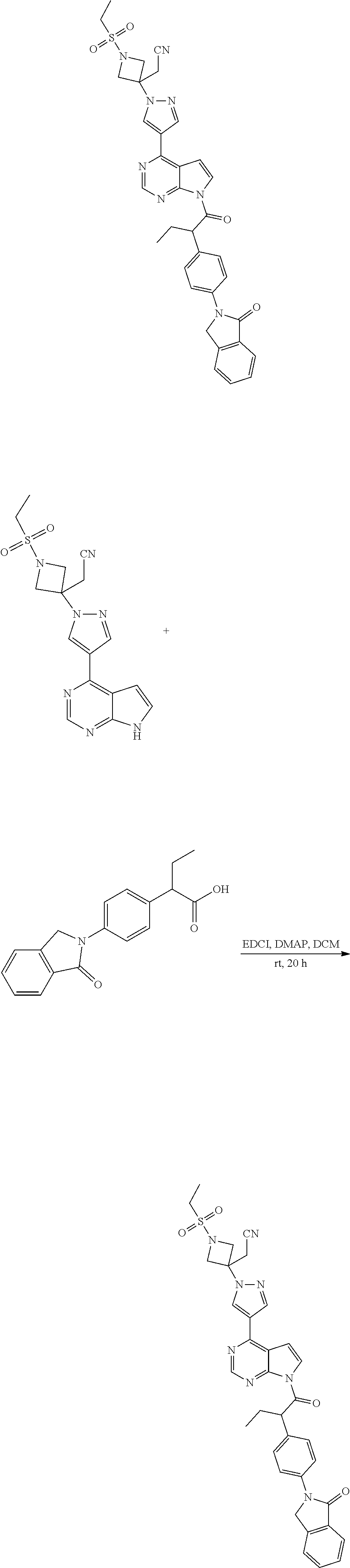 [SMILES: CCC(C(=O)n1ccc2c(-c3cnn(C4(CC#N)CN(S(=O)(=O)CC)C4)c3)ncnc21)c1ccc(N2Cc3ccccc3C2=O)cc1 ; CCS(=O)(=O)N1CC(CC#N)(n2cc(-c3ncnc4[nH]ccc34)cn2)C1 ; CCC(C(=O)O)c1ccc(N2Cc3ccccc3C2=O)cc1 ; CCC(C(=O)n1ccc2c(-c3cnn(C4(CC#N)CN(S(=O)(=O)CC)C4)c3)ncnc21)c1ccc(N2Cc3ccccc3C2=O)cc1]

1-(Ethylsulfonyl)-3-[4-(7H-pyrrolo [2, 3-d] pyrimidin-4-yl)-1H-pyrazol-1-yl]-3-azetidine acetonitrile (baricitinib, 371 mg, 1 mmol), 4-dimethylamino pyridine (DMAP, 61 mg, 0.5 mmol), 2-(4-(1-oxoisoindolin-2-yl) phenyl) butanoic acid (indobufen, 354 mg, 1.2 mmol) and 1-(3-dimethylaminopropyl)-3-ethylcarbodiimide hydrochloride (EDCI, 288 mg, 1.5 mmol) was dissolved in dichloromethane (15 mL) and stirred at room temperature for 20 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:1) to give the title compound as a white solid, 0.46 g, 70.9% yield. MS (m/z): [M+H]⁺ calcd for C₃₄H₃₂N₈O₄S, 649.74; found, 649.3. ¹H NMR (400 MHz, DMSO) δ 9.05 (s, 1H), 8.98 (s, 1H), 8.50 (s, 1H), 8.16 (d, J=3.9 Hz, 1H), 7.86 (d, J=8.4 Hz, 2H), 7.73 (t, J=14.0 Hz, 1H), 7.69-7.58 (m, 2H), 7.52 (d, J=8.4 Hz, 3H), 7.40 (d, J=3.9 Hz, 1H), 5.85 (t, J=7.2 Hz, 1H), 4.96 (s, 2H), 4.59 (d, J=9.0 Hz, 2H), 4.24 (d, J=9.0 Hz, 2H), 3.68 (s, 2H), 3.22 (dd, J=14.5, 7.2 Hz, 2H), 2.26 (dt, J=13.8, 7.1 Hz, 1H), 1.94 (dd, J=13.6, 7.2 Hz, 1H), 1.23 (dd, J=14.8, 7.6 Hz, 3H), 0.94 (t, J=7.1 Hz, 3H).

### Example 31

### (R)-3-cyclopentyl-3-(4-(7-((S)-2-(6-methoxynaphthalen-2-yl) propanoyl)-7H-pyrrolo [2, 3-d] pyrimidin-4-yl))-1H-pyrazol-1-yl) propanenitrile

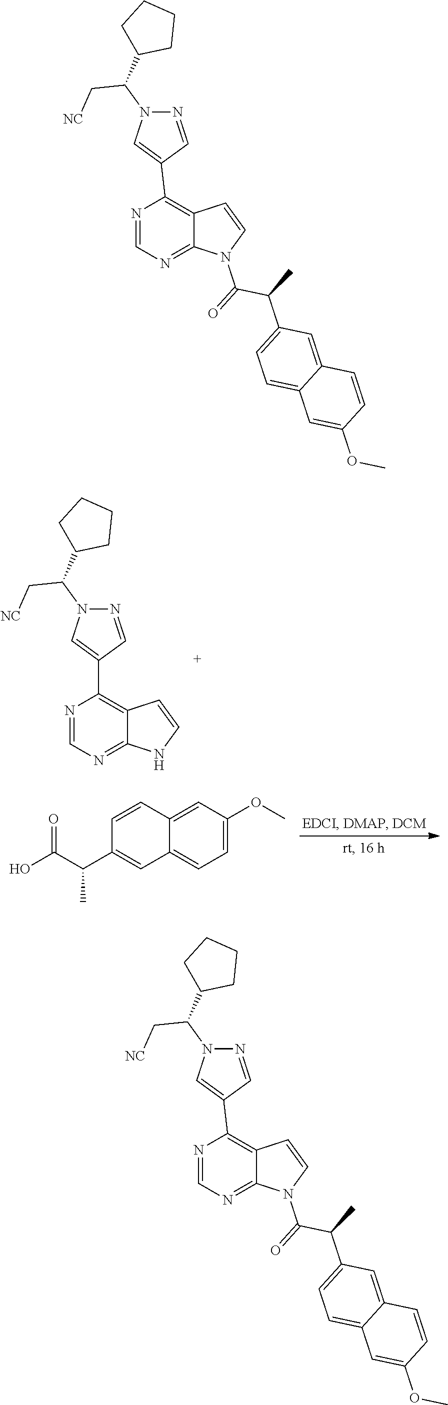 [SMILES: COc1ccc2cc([C@H](C)C(=O)n3ccc4c(-c5cnn([C@H](CC#N)C6CCCC6)c5)ncnc43)ccc2c1 ; N#CC[C@H](C1CCCC1)n1cc(-c2ncnc3[nH]ccc23)cn1 ; COc1ccc2cc([C@H](C)C(=O)O)ccc2c1 ; COc1ccc2cc([C@H](C)C(=O)n3ccc4c(-c5cnn([C@H](CC#N)C6CCCC6)c5)ncnc43)ccc2c1]

(R)-3-(4-(7h-pyrrolo [2, 3-d] pyridin-4-yl)-1h-pyrazol-1-yl)-3-cyclopentylpropane (Ruxolitinib, 153 mg, 0.5 mmol), 4-dimethylamino pyridine (DMAP, 6 mg, 0.05 mmol), (S)-(+)-2-(6-methoxy-2-naphthyl) propanoic acid (naproxen, 138 mg, 0.6 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=1:1 to 1:2) to give the title compound as a white solid, 0.16 g, 61.7% yield. MS (m/z): [M+H]⁺ calcd for C₃₁H₃₀N₆O₂, 519.62; found, 519.3. ¹H NMR (400 MHz, DMSO) δ 8.99 (s, 1H), 8.85 (s, 1H), 8.39 (s, 1H), 8.14 (d, J=4.2 Hz, 1H), 7.84 (s, 1H), 7.81-7.73 (m, 2H), 7.55 (dd, J=8.6, 1.5 Hz, 1H), 7.30 (d, J=4.2 Hz, 1H), 7.24 (d, J=2.2 Hz, 1H), 7.11 (dd, J=9.0, 2.4 Hz, 1H), 6.13 (q, J=6.8 Hz, 1H), 4.53 (td, J=9.6, 4.2 Hz, 1H), 3.83 (s, 3H), 3.29-3.14 (m, 2H), 2.47-2.34 (m, 1H), 1.81 (td, J=11.7, 7.3 Hz, 1H), 1.68 (d, J=6.9 Hz, 3H), 1.63-1.38 (m, 4H), 1.38-1.21 (m, 3H).

### Example 32

### (3R)-3-(4-(7-(2-(3-benzoylphenyl) propanoyl)-7H-pyrrolo [2, 3-d] pyrimidin-4-yl)-1H-pyrazol-1-yl)-3-cyclopentylpropanenitrile

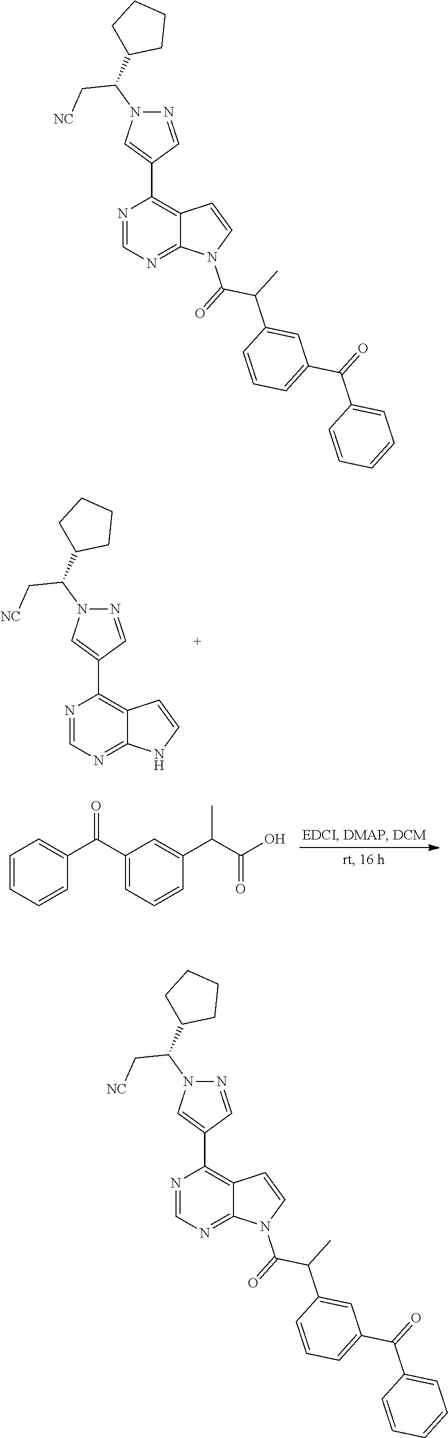

(R)-3-(4-(7h-pyrrolo [2, 3-d] pyridin-4-yl)-1h-pyrazol-1-yl)-3-cyclopentylpropane (Ruxolitinib, 153 mg, 0.5 mmol), 4-dimethylamino pyridine (DMAP, 6 mg, 0.05 mmol), 2-(3-benzoylphenyl) propanoic acid (ketoprofen, 152 mg, 0.6 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=10:1 to 1:1) to give the title compound as a white solid, 0.18 g, 66.4% yield. MS (m/z): [M+H]⁺ calcd for C₃₃H₃₀N₆O₂, 543.64; found, 543.2. ¹H NMR (400 MHz, DMSO) δ 8.89 (d, J=6.4 Hz, 2H), 8.41 (s, 1H), 8.14 (d, J=4.2 Hz, 1H), 7.82 (s, 1H), 7.76 (d, J=7.7 Hz, 1H), 7.70-7.59 (m, 4H), 7.53 (t, J=7.6 Hz, 3H), 7.33 (d, J=4.1 Hz, 1H), 6.08 (q, J=6.8 Hz, 1H), 4.55 (td, J=9.6, 4.2 Hz, 1H), 3.29-3.16 (m, 2H), 2.42 (dt, J=17.0, 8.5 Hz, 1H), 1.82 (td, J=11.6, 7.3 Hz, 1H), 1.70-1.39 (m, 7H), 1.39-1.20 (m, 3H).

### Example 33

### (3R)-3-cyclopentyl-3-(4-(7-(2-(2-fluoro-[1, 1′-biphenyl]-4-yl) propanoyl)-7H-pyrrolo [2, 3-d] pyrimidin-4-yl)-1H-pyrazol-1-yl) propanenitrile

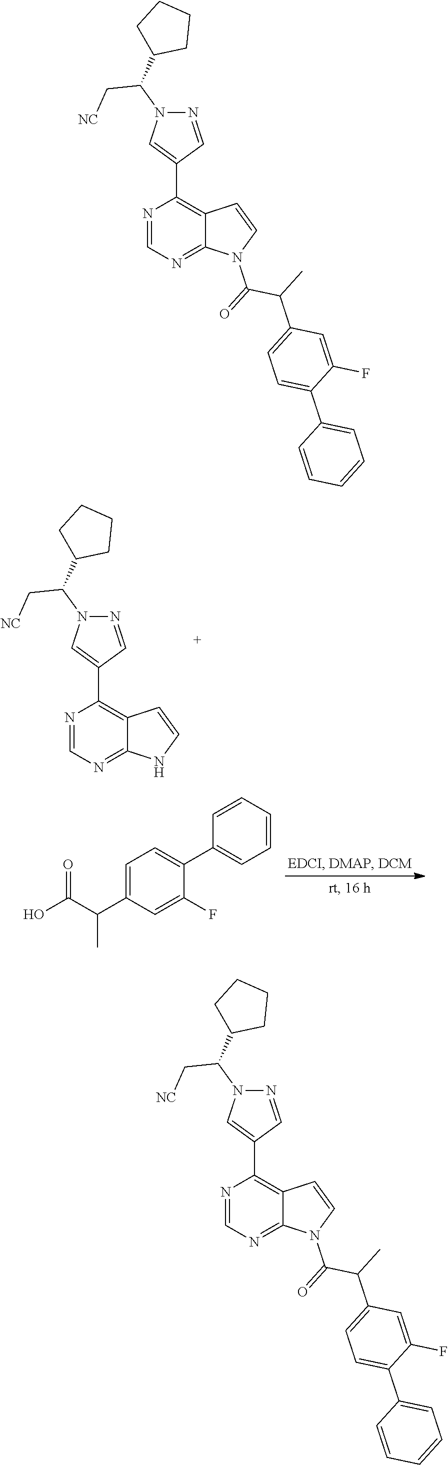 [SMILES: CC(C(=O)n1ccc2c(-c3cnn([C@H](CC#N)C4CCCC4)c3)ncnc21)c1ccc(-c2ccccc2)c(F)c1 ; N#CC[C@H](C1CCCC1)n1cc(-c2ncnc3[nH]ccc23)cn1 ; CC(C(=O)O)c1ccc(-c2ccccc2)c(F)c1 ; CC(C(=O)n1ccc2c(-c3cnn([C@H](CC#N)C4CCCC4)c3)ncnc21)c1ccc(-c2ccccc2)c(F)c1]

(R)-3-(4-(7h-pyrrolo [2, 3-d] pyridin-4-yl)-1h-pyrazol-1-yl)-3-cyclopentylpropane (Ruxolitinib, 153 mg, 0.5 mmol), 4-dimethylamino pyridine (DMAP, 6 mg, 0.05 mmol), 2-(2-fluoro-4-biphenyl) propanoic acid (flurbiprofen, 146 mg, 0.6 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 16 hours. After completion of the reaction, the reaction solution was diluted with dichloromethane and washed with water and saturated saltwater solution. The organic layer was dried over anhydrous sodium sulfate and filtered. The solvent was evaporated under reduced pressure to obtain the crude product. The crude product was further separated by silica gel column chromatography (petroleum ether/ethyl acetate=1:1 to 1:2) to give the title compound as a white solid, 0.17 g, 63.9% yield. MS (m/z): [M+H]⁺ calcd for C₃₂H₂₉FN₆O, 533.62; found, 533.2.1H NMR (400 MHz, DMSO) δ 9.00 (s, 1H), 8.88 (s, 1H), 8.41 (s, 1H), 8.15 (d, J=4.1 Hz, 1H), 7.52-7.29 (m, 9H), 6.08 (q, J=6.7 Hz, 1H), 4.54 (td, J=9.4, 4.0 Hz, 1H), 3.28-3.15 (m, 2H), 2.43 (dd, J=16.9, 8.4 Hz, 1H), 1.87-1.76 (m, 1H), 1.70-1.38 (m, 7H), 1.38-1.20 (m, 3H).

### Example 34

### (3R)-3-cyclopentyl-3-(4-(7-(2-(4-(1-oxoisoindolin-2-yl) phenyl) butanoyl)-7H-pyrrolo [2, 3-d] pyrimidin-4-yl)-1H-pyrazol-1-yl) propanenitrile

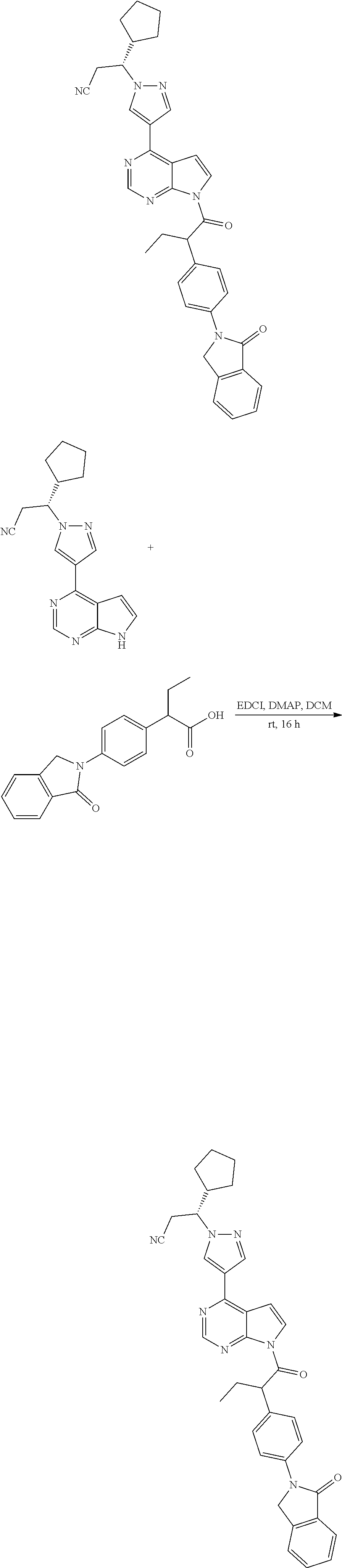 [SMILES: CCC(C(=O)n1ccc2c(-c3cnn([C@H](CC#N)C4CCCC4)c3)ncnc21)c1ccc(N2Cc3ccccc3C2=O)cc1 ; N#CC[C@H](C1CCCC1)n1cc(-c2ncnc3[nH]ccc23)cn1 ; CCC(C(=O)O)c1ccc(N2Cc3ccccc3C2=O)cc1 ; CCC(C(=O)n1ccc2c(-c3cnn([C@H](CC#N)C4CCCC4)c3)ncnc21)c1ccc(N2Cc3ccccc3C2=O)cc1]

(R)-3-(4-(7h-pyrrolo [2, 3-d] pyridin-4-yl)-1h-pyrazol-1-yl)-3-cyclopentylpropane (ruxolitinib, 153 mg, 0.5 mmol), 4-dimethylamino pyridine (DMAP, 6 mg, 0.05 mmol), 2-(4-(1-oxoisoindolin-2-yl) phenyl) butanoic acid (indobufen, 177 mg, 0.6 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:1) to give the title compound as a white solid, 0.17 g, 58.3% yield. MS (m/z): [M+H]⁺ calcd for C₃₅H₃₃N₇O₂, 584.70; found, 584.3. ¹H NMR (400 MHz, DMSO) δ 9.01 (s, 1H), 8.86 (s, 1H), 8.39 (s, 1H), 8.13 (d, J=4.2 Hz, 1H), 7.86 (d, J=8.7 Hz, 2H), 7.75 (d, J=7.6 Hz, 1H), 7.70-7.59 (m, 2H), 7.52 (t, J=7.0 Hz, 3H), 7.30 (d, J=3.9 Hz, 1H), 5.86 (t, J=7.4 Hz, 1H), 4.96 (s, 2H), 4.53 (td, J=9.5, 4.2 Hz, 1H), 3.28-3.14 (m, 2H), 2.41 (dt, J=17.2, 8.5 Hz, 1H), 2.25 (td, J=14.2, 7.2 Hz, 1H), 1.97-1.88 (m, 1H), 1.80 (dt, J=11.9, 5.8 Hz, 1H), 1.65-1.39 (m, 3H), 1.36-1.21 (m, 4H), 0.94 (t, J=7.3 Hz, 3H).

### Example 35

### N-methyl-1-((trans)-4-(methyl (7-(2-(4-(1-oxoisoindolin-2-yl) phenyl) butanoyl)-7H-pyrrolo [2, 3-d] pyrimidin-4-yl) amino) cyclohexyl) methanesulfonamide

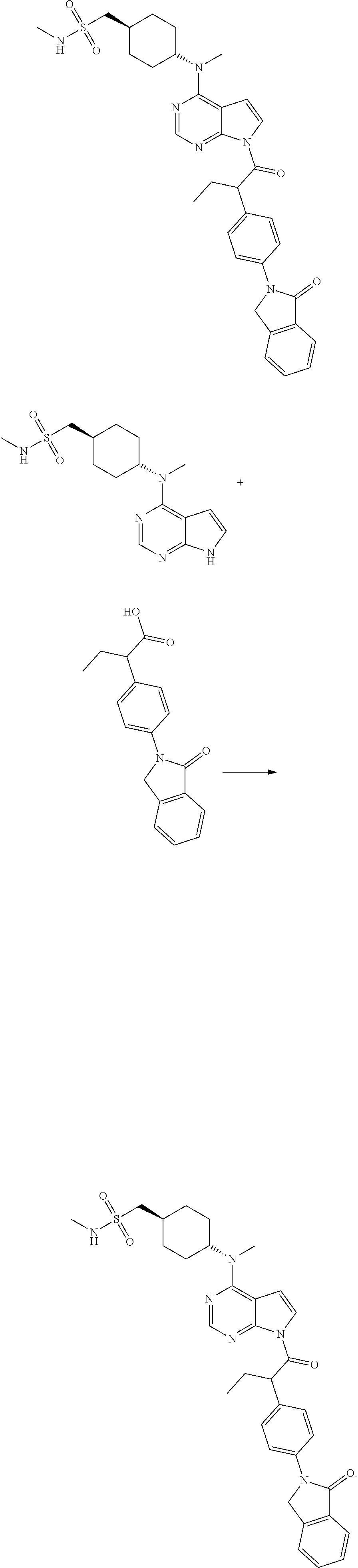

(N-methyl-1-((trans)-4-(methyl (7H-pyrrolo [2, 3-d] pyrimidin-4-yl) amino) cyclohexyl) methanesulfonamide (oclacitinib, 203 mg, 0.6 mmol), 4-dimethylamino pyridine (DMAP, 73 mg, 0.6 mmol), 2-(4-(1-oxoisoindolin-2-yl) phenyl) butanoic acid (indobufen, 230 mg, 0.78 mmol) and 1-(3-dimethylaminopropyl)-3-ethylcarbodiimide hydrochloride (EDCI, 173 mg, 0.9 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:1) to give the title compound as a white solid, 0.22 g, 59.7% yield. MS (m/z): [M+H]⁺ calcd for C₃₃H₃₈N₆O₄S, 615.77; found, 615.3. ¹H NMR (400 MHz, DMSO) δ 8.41 (s, 1H), 7.83 (d, J=8.3 Hz, 2H), 7.76 (d, J=7.4 Hz, 1H), 7.67 (dd, J=9.6, 5.5 Hz, 3H), 7.59-7.42 (m, 3H), 6.92-6.73 (m, 2H), 5.97 (t, J=7.1 Hz, 1H), 4.96 (s, 2H), 4.64 (s, 1H), 3.14 (s, 3H), 2.94 (d, J=5.8 Hz, 2H), 2.57 (t, J=9.7 Hz, 3H), 2.26-2.13 (m, 1H), 2.03 (d, J=12.0 Hz, 2H), 1.97-1.77 (m, 2H), 1.68 (s, 4H), 1.37-1.13 (m, 2H), 0.90 (dd, J=18.8, 11.8 Hz, 3H).

### Example 36

### 1-((Trans)-4-((7-(2-(2-fluoro-[1, 1′-biphenyl]-4-yl) propanoyl)-7H-pyrrolo [2, 3-d] pyrimidin-4-yl) (methyl) amino) cyclohexyl)-N-methyl methanesulfonamide

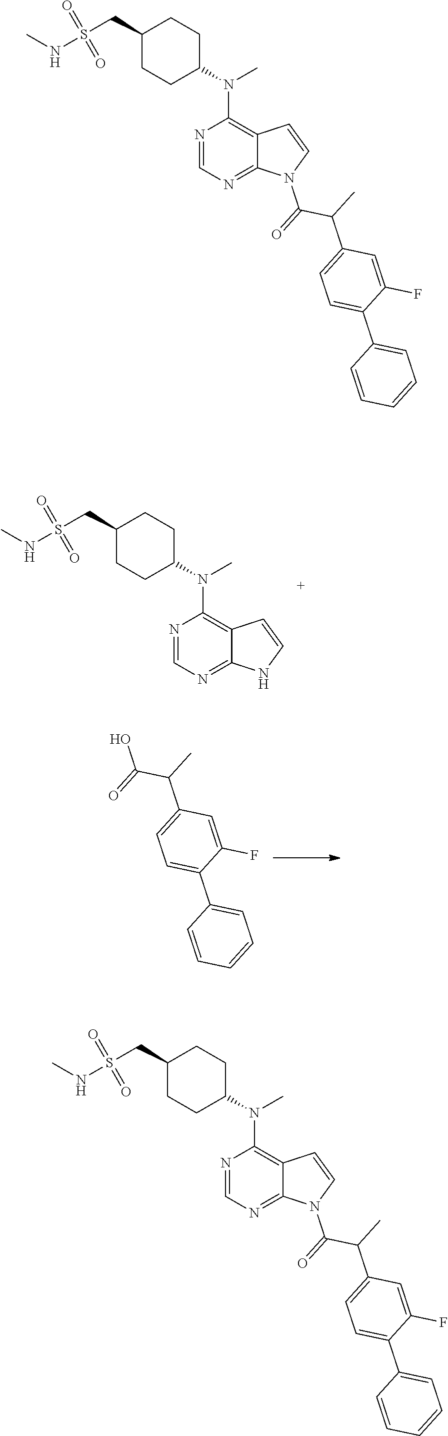

N-methyl-1-((trans)-4-(methyl (7H-pyrrolo [2, 3-d] pyrimidin-4-yl) amino) cyclohexyl) methane sulfonamide (oclacitinib, 203 mg, 0.6 mmol), 4-dimethylamino pyridine (DMAP, 73 mg, 0.6 mmol), 2-(2-fluoro-4-biphenyl) propanoic acid (flurbiprofen, 191 mg, 0.78 mmol) and 1-(3-dimethylaminopropyl)-3-ethylcarbodiimide hydrochloride (EDCI, 173 mg, 0.9 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=1:1 to 1:2) to give the title compound as a white solid, 0.22 g, 65.1% yield. MS (m/z): [M+H]⁺ calcd for C₃₀H₃₄FN₅O₃S, 564.69; found, 564.3. ¹H NMR (400 MHz, DMSO) δ 8.39 (s, 1H—), 7.70 (d, J=4.2 Hz, 1H—), 7.52-7.41 (m, 5H), 7.35 (dt, J=19.2, 7.4 Hz, 3H), 6.92-6.82 (m, 2H), 6.17 (q, J=6.8 Hz, 1H—), 4.64 (s, 1H—), 3.16 (s, 3H), 2.94 (d, J=6.2 Hz, 2H), 2.59 (d, J=4.9 Hz, 3H), 2.04 (d, J=12.5 Hz, 2H), 1.85 (s, 1H—), 1.65 (d, J=26.0 Hz, 4H), 1.59 (d, J=7.0 Hz, 3H), 1.37-1.19 (m, 2H).

### Example 37

### 1-((Trans)-4-((7-(2-(1, 8-diethyl-1, 3, 4, 9-tetrahydropyrano [3, 4-b] indol-1-yl) acetyl)-7H-pyrrolo [2, 3-d] pyrimidin-4-yl) (methyl) amino) cyclohexyl)-N-methyl methanesulfonamide

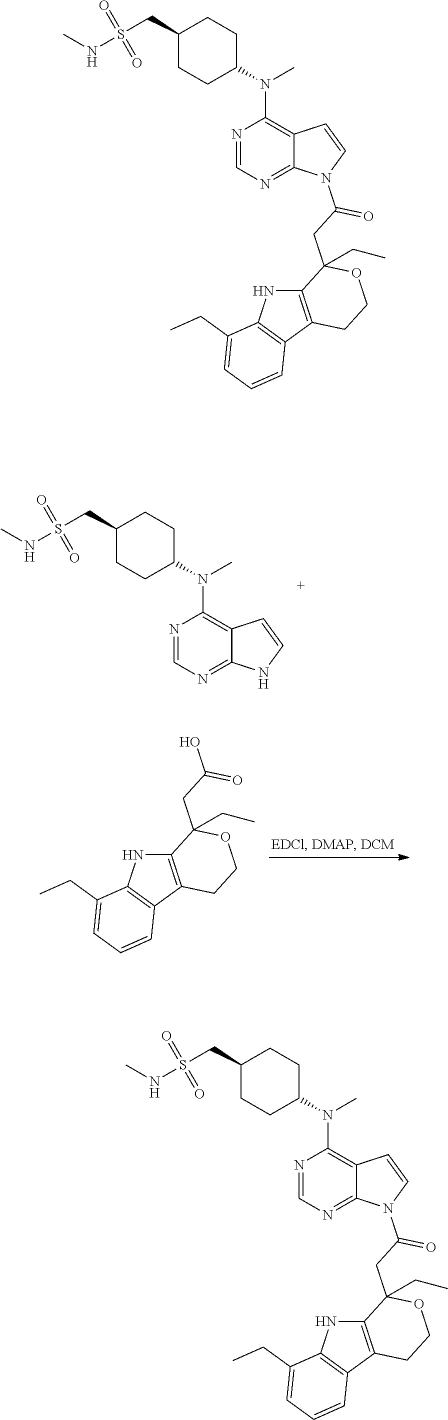

N-methyl-1-((trans)-4-(methyl (7H-pyrrolo [2, 3-d] pyrimidin-4-yl) amino) cyclohexyl) methanesulfonamide (oclacitinib, 203 mg, 0.6 mmol), 4-dimethylamino pyridine (DMAP, 73 mg, 0.6 mmol), 1, 8-diethyl-1, 3, 4, 9-tetrahydropyran [3, 4-b] indole-1-acetic acid (etodolac, 224 mg, 0.78 mmol) and 1-(3-dimethylaminopropyl)-3-ethylcarbodiimide hydrochloride (EDCI, 173 mg, 0.9 mmol) was dissolved in dichloromethane (15 mL) and stirred at room temperature for 3 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=3:1 to 1:1) to give the title compound as a white solid, 0.23 g, 63.2% yield. MS (m/z): [M+H]⁺ calcd for C₃₂H₄₂N₆O₄S, 607.79; found, 607.3. ¹H NMR (400 MHz, DMSO-d₆) δ 10.47 (s, 1H), 8.27 (s, 1H), 7.64 (d, J=4.2 Hz, 1H), 7.22 (dd, J=7.4, 1.6 Hz, 1H), 6.98-6.78 (m, 4H), 4.66 (d, J=14.6 Hz, 2H), 4.03 (d, J=7.5 Hz, 1H), 3.89 (s, 1H), 3.76 (dt, J=10.7, 4.4 Hz, 1H), 3.16 (s, 3H), 2.94 (d, J=6.2 Hz, 2H), 2.84 (q, J=7.5 Hz, 2H), 2.59 (t, J=3.8 Hz, 5H), 2.15 (tt, J=7.2, 4.6 Hz, 2H), 2.09-2.01 (m, 2H), 1.85 (t, J=7.5 Hz, 1H), 1.69 (s, 4H), 1.35-1.21 (m, 5H), 0.68 (t, J=7.3 Hz, 3H).

### Example 38

### 1-((Trans)-4-((7-(2-(3-benzoylphenyl) propanoyl)-7H-pyrrolo [2, 3-d] pyrimidin-4-yl) (methyl) amino) cyclohexyl)-N-methyl methanesulfonamide

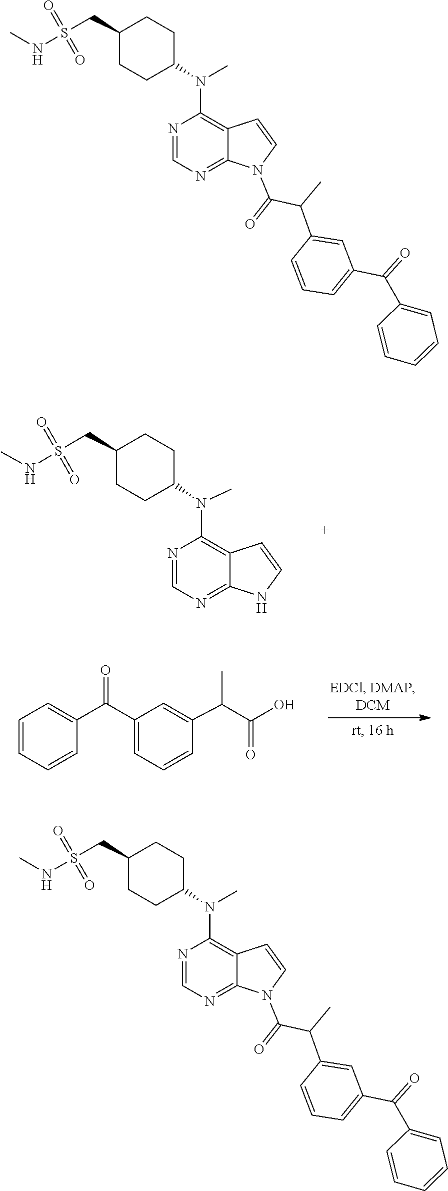

N-methyl-1-((trans)-4-(methyl (7H-pyrrolo [2, 3-d] pyrimidin-4-yl) amino) cyclohexyl) methanesulfonamide (oclacitinib, 203 mg, 0.6 mmol), 4-dimethylamino pyridine (DMAP, 73 mg, 0.6 mmol), 2-(3-benzoylphenyl) propanoic acid (ketoprofen, 198 mg, 0.78 mmol) and 1-(3-dimethylaminopropyl)-3-ethylcarbodiimide hydrochloride (EDCI, 173 mg, 0.9 mmol) were dissolved in dichloromethane (15 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:1) to give the title compound as a white solid, 0.17 g, 49.4% yield. MS (m/z): [M+H]⁺ calcd for C₃₁H₃₅N₅O₄S, 574.71; found, 574.3. ¹H NMR (400 MHz, DMSO-d₆) δ 8.30 (s, 1H), 7.78 (t, J=1.9 Hz, 1H), 7.74-7.57 (m, 6H), 7.52 (dt, J=11.6, 7.7 Hz, 3H), 6.86 (t, J=4.9 Hz, 2H), 6.16 (q, J=6.9 Hz, 1H), 4.64 (s, 1H), 3.15 (s, 3H), 2.95 (d, J=6.2 Hz, 2H), 2.59 (d, J=5.0 Hz, 3H), 2.04 (d, J=13.0 Hz, 2H), 1.84 (dq, J=13.3, 6.8 Hz, 1H), 1.68 (d, J=8.5 Hz, 4H), 1.58 (d, J=7.0 Hz, 3H), 1.28 (s, 2H).

### Example 39

### 1-((Trans)-4-((7-((S)-2-(6-methoxynaphthalen-2-yl) propanoyl)-7H-pyrrolo [2, 3-d] pyrimidin-4-yl) (methyl) amino) cyclohexyl)-N-methyl methanesulfonamide

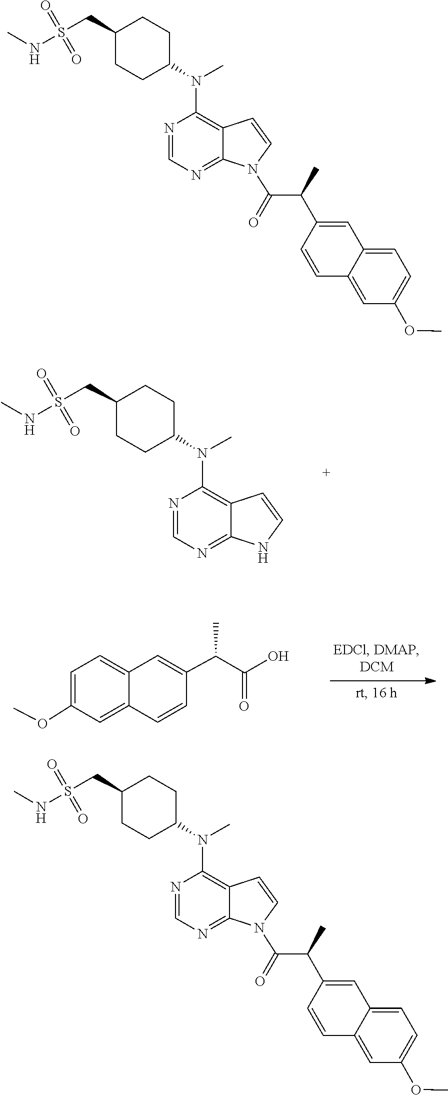

N-Methyl-1-((trans)-4-(methyl (7H-pyrrolo [2, 3-d] pyrimidin-4-yl) amino) cyclohexyl) methanesulfonamide (oclacitinib, 203 mg, 0.6 mmol), 4-dimethylamino pyridine (DMAP, 73 mg, 0.6 mmol), (S)-(+)-2-(6-methoxy-2-naphthyl) propanoic acid (naproxen, 180 mg, 0.78 mmol) and 1-(3-dimethylaminopropyl)-3-ethylcarbodiimide hydrochloride (EDCI, 173 mg, 0.9 mmol) were dissolved in dichloromethane (15 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:1) to give the title compound as a white solid, 0.23 g, 69.8% yield. MS (m/z): [M+H]⁺ calcd for C₂₉H₃₅N₅O₄S, 550.69; found, 550.4. ¹H NMR (400 MHz, DMSO-d₆) δ 8.39 (s, 1H), 7.78 (d, J=7.8 Hz, 1H), 7.74 (t, J=8.9 Hz, 2H), 7.68 (d, J=4.1 Hz, 1H), 7.50 (dd, J=8.5, 1.3 Hz, 1H), 7.23 (d, J=2.1 Hz, 1H), 7.11 (dd, J=8.9, 2.4 Hz, 1H), 6.87 (q, J=4.5 Hz, 1H), 6.79 (d, J=3.0 Hz, 1H), 6.21 (q, J=6.8 Hz, 1H), 4.61 (s, 1H), 3.83 (s, 3H), 3.10 (s, 3H), 2.93 (d, J=6.2 Hz, 2H), 2.59 (d, J=4.8 Hz, 3H), 2.06-1.98 (m, 2H), 1.83 (s, 1H), 1.63 (t, J=9.3 Hz, 7H), 1.36-1.15 (m, 2H).

### Example 40

### 2-(3-(4-(7-(2-((3-Chloro-2-methylphenyl) amino) benzoyl)-7H-pyrrolo [2, 3-d] pyrimidin-4-yl)-1H-pyrazol-1-yl)-1-(ethylsulfonyl) azetidin-3-yl) acetonitrile

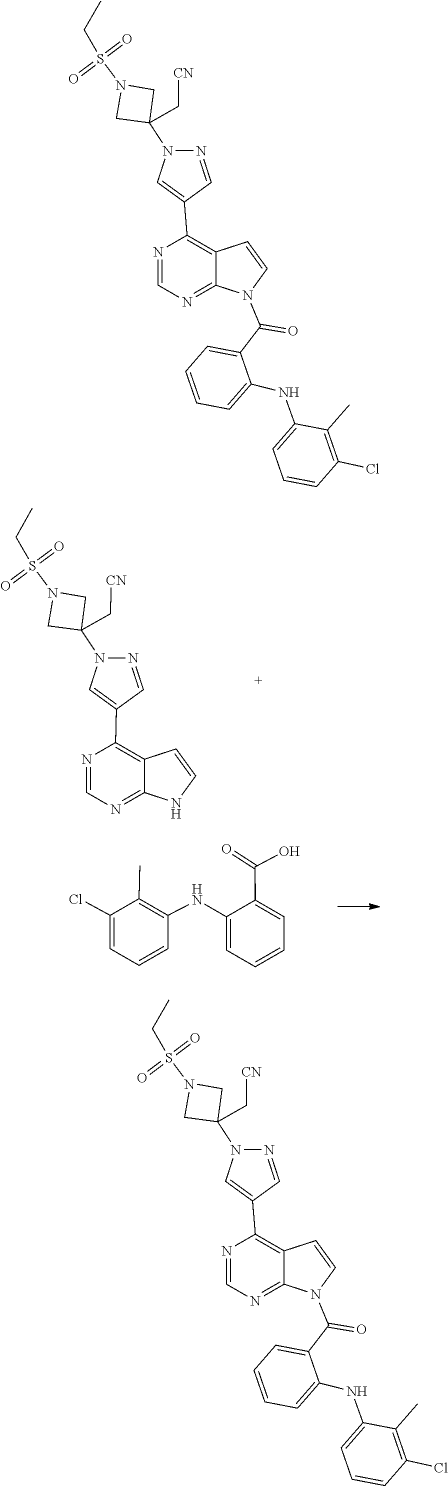

1-(Ethylsulfonyl)-3-[4-(7H-pyrrolo [2, 3-d] pyrimidin-4-yl)-1H-pyrazol-1-yl]-3-azetidine acetonitrile (baricitinib, 371 mg, 1 mmol), 4-dimethylamino pyridine (DMAP, 61 mg, 0.5 mmol), 2-((3-chloro-2-methylphenyl) amino) benzoic acid (tolfenamic acid, 313 mg, 1.2 mmol) and 1-(3-dimethylaminopropyl)-3-ethylcarbodiimide hydrochloride (EDCI, 288 mg, 1.5 mmol) were dissolved in dichloromethane (10 mL) and stirred at room temperature for 24 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:1) to give the title compound as a white solid, 0.22 g, 35.7% yield. MS (m/z): [M+H]⁺ calcd for C₃₀H₂₇ClN₈O₃S, 616.11; found, 616.3. ¹H NMR (400 MHz, DMSO-d₆) δ 8.99 (s, 1H), 8.73 (s, 1H), 8.51 (s, 1H), 8.21 (s, 1H), 7.83 (d, J=4.1 Hz, 1H), 7.50 (dd, J=8.3, 6.5 Hz, 2H), 7.37 (d, J=4.1 Hz, 1H), 7.09-7.04 (m, 3H), 7.00-6.90 (m, 2H), 4.61 (d, J=9.1 Hz, 2H), 4.25 (d, J=9.1 Hz, 2H), 3.70 (s, 2H), 3.24 (q, J=7.3 Hz, 2H), 2.01 (s, 3H), 1.25 (t, J=7.4 Hz, 3H).

### Example 41

### 1-((Trans)-4-((7-(2-((3-chloro-2-methylphenyl) amino) benzoyl)-7H-pyrrolo [2, 3-d] pyrimidin-4-yl) (methyl) amino) cyclohexyl)-N-methyl methanesulfonamide

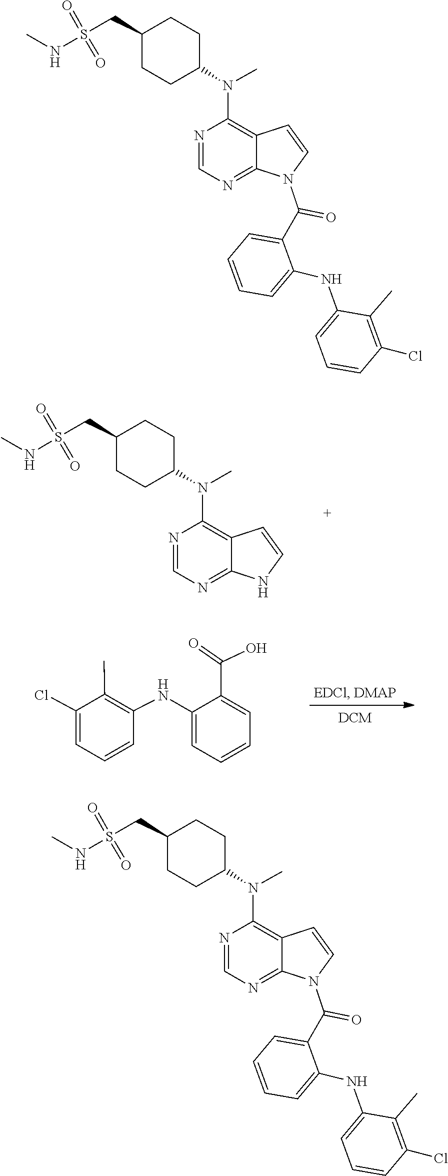

N-methyl-1-((trans)-4-(methyl (7H-pyrrolo [2, 3-d] pyrimidin-4-yl) amino) cyclohexyl) methanesulfonamide (oclacitinib, 203 mg, 0.6 mmol), 4-dimethylamino pyridine (DMAP, 73 mg, 0.6 mmol), 2-((3-chloro-2-methylphenyl) amino) benzoic acid (tolfenamic acid, 204 mg, 0.78 mmol) and 1-(3-dimethylaminopropyl)-3-ethylcarbodiimide hydrochloride (EDCI, 173 mg, 0.9 mmol) were dissolved in dichloromethane (15 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further purified by silica gel column chromatography (petroleum ether/ethyl acetate=2:1 to 1:1) to give the title compound as a yellow solid, 0.2 g, 57.4% yield. MS (m/z): [M+H]⁺ calcd for C₂₉H₃₃ClN₆O₃S, 581.20; found, 581.4. ¹H NMR (400 MHz, DMSO-d₆) δ 8.24 (s, 1H), 8.08 (s, 1H), 7.45 (dd, J=11.4, 4.2 Hz, 1H), 7.41-7.22 (m, 2H), 7.14-7.01 (m, 3H), 6.96 (d, J=8.3 Hz, 1H), 6.93-6.75 (m, 3H), 4.63 (s, 1H), 3.17 (s, 3H), 2.95 (d, J=6.1 Hz, 2H), 2.60 (d, J=4.9 Hz, 3H), 2.13-2.01 (s, 5H), 1.89 (d, J=22.2 Hz, 1H), 1.78-1.62 (m, 4H), 1.36-1.24 (m, 2H).

### Example 42

### 1-((Trans)-4-((7-(2-(1-(4-chlorobenzoyl)-5-methoxy-2-methyl-1H-indol-3-yl) acetyl)-7H-pyrrolo [2, 3-d] pyrimidin-4-yl) (methyl) amino) cyclohexyl)-N-methylmethanesulfonamide

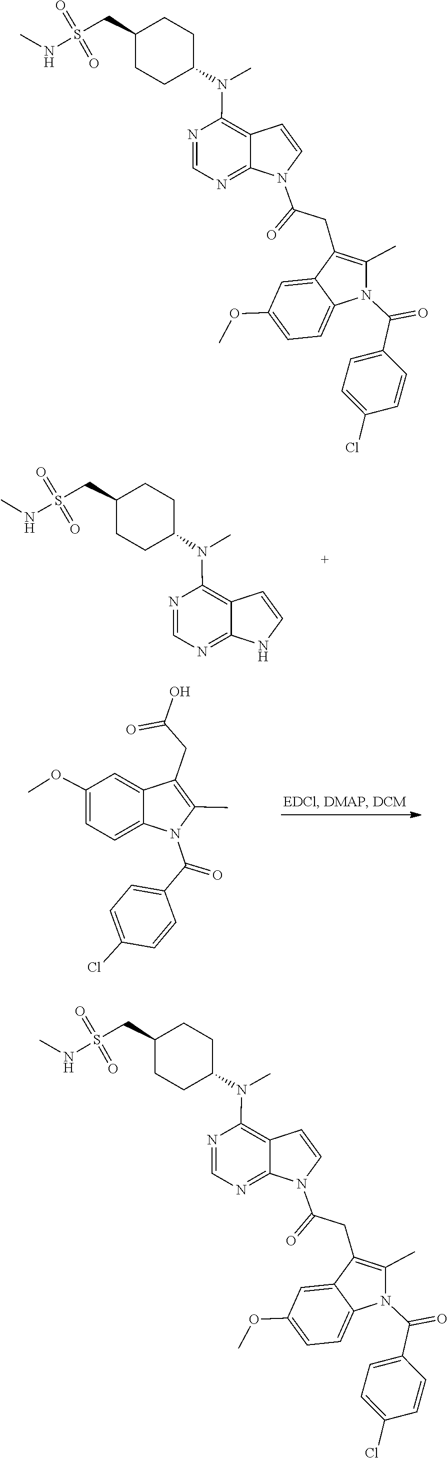

### Synthesis of 1-((trans)-4-((7-(2-(1-(4-chlorobenzoyl)-5-methoxy-2-methyl-1H-indol-3-yl) acetyl)-7H-pyrrolo [2, 3-d] pyrimidin-4-yl) (methyl) amino) cyclohexyl)-N-methyl methanesulfonamide

N-methyl-1-((trans)-4-(methyl (7H-pyrrolo [2, 3-d] pyrimidin-4-yl) amino) cyclohexyl) methanesulfonamide (oclacitinib, 168 mg, 0.5 mmol), 4-dimethylamino pyridine (DMAP, 61 mg, 0.5 mmol), 1-(4-chlorobenzoyl)-5-methoxy-2-methyl-1H-indole-3-acetic acid (indomethacin, 233 mg, 0.65 mmol) and 1-(3-dimethylaminopropyl)-3-ethylcarbodiimide hydrochloride (EDCI, 144 mg, 0.75 mmol) was dissolved in dichloromethane (15 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further purified by silica gel column chromatography (petroleum ether/ethyl acetate=2:1 to 1:1) to give the title compound as a yellow solid, 0.14 g, 41.4% yield. MS (m/z): [M+H]⁺ calcd for C₃₄H₃₇ClN₆O₅S, 677.22; found, 677.3. ¹H NMR (400 MHz, DMSO-d₆) δ 8.42 (s, 1H), 7.76-7.60 (m, 5H), 7.11 (d, J=2.6 Hz, 1H), 6.98 (d, J=9.0 Hz, 1H), 6.89 (dt, J=9.9, 4.5 Hz, 2H), 6.71 (dd, J=9.0, 2.6 Hz, 1H), 4.99 (s, 2H), 4.69 (s, 1H), 3.69 (s, 3H), 3.20 (s, 3H), 2.96 (d, J=6.2 Hz, 2H), 2.59 (d, J=4.9 Hz, 3H), 2.24 (s, 3H), 2.06 (d, J=12.6 Hz, 2H), 1.87 (dt, J=15.5, 5.8 Hz, 1H), 1.72 (dt, J=8.5, 5.0 Hz, 4H), 1.41-1.27 (m, 2H).

### Example 43

### 1-((trans)-4-((7-(2-((2, 3-dimethylphenyl) amino) benzoyl)-7H-pyrrolo [2, 3-d] pyrimidin-4-yl) (methyl) amino) cyclohexyl)-N-methyl methanesulfonamide

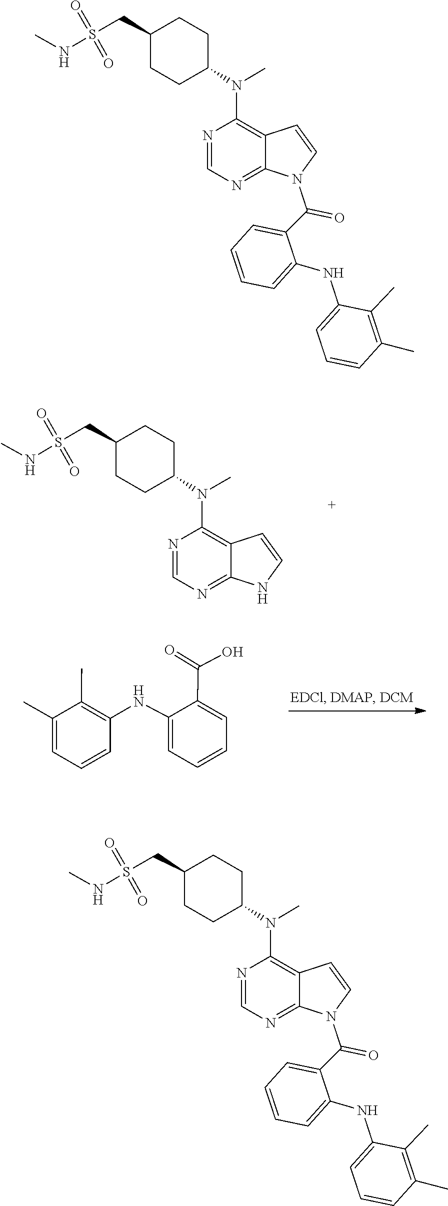

### Synthesis of 1-((trans)-4-((7-(2-((2, 3-dimethylphenyl) amino) benzoyl)-7H-pyrrolo [2, 3-d] pyrimidin-4-yl) (methyl) amino) cyclohexyl)-N-methyl methanesulfonamide

N-methyl-1-((trans)-4-(methyl (7H-pyrrolo [2, 3-d] pyrimidin-4-yl) amino) cyclohexyl) methanesulfonamide (oclacitinib, 168 mg, 0.5 mmol), 4-dimethylamino pyridine (DMAP, 61 mg, 0.5 mmol), 2-((2, 3-dimethylphenyl) amino) benzoic acid (mefenamic acid, 157 mg, 0.65 mmol), and 1-(3-dimethylaminopropyl)-3-ethylcarbodiimide hydrochloride (EDCI, 144 mg, 0.75 mmol) were dissolved in dichloromethane (15 mL) and stirred at room temperature for 7 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further purified by silica gel column chromatography (petroleum ether/ethyl acetate=2:1 to 1:1) to give the title compound as a yellow solid, 0.17 g, 60.7% yield. MS (m/z): [M+H]⁺ calcd for C₃₀H₃₆N₆O₃S, 561.26; found, 561.3. ¹H NMR (400 MHz, DMSO-d₆) δ 8.59 (s, 1H), 8.12 (s, 1H), 7.48 (d, J=4.0 Hz, 1H), 7.43-7.28 (m, 2H), 7.13-6.96 (m, 3H), 6.92-6.79 (m, 3H), 6.77-6.66 (m, 1H), 4.82-4.49 (m, 1H), 3.19 (s, 3H), 2.95 (d, J=6.2 Hz, 2H), 2.59 (d, J=4.9 Hz, 3H), 2.27 (d, J=9.8 Hz, 3H), 2.07 (d, J=21.6 Hz, 5H), 1.92-1.82 (m, 1H), 1.72 (h, J=3.3 Hz, 4H), 1.37-1.26 (m, 2H).

### Example 44

### N-methyl-1-((trans)-4-(methyl (7-(2-(4-((2-oxocyclopentyl) methyl) phenyl) propanoyl)-7H-pyrrolo [2, 3-d] pyrimidin-4-yl) amino) cyclohexyl) methanesulfonamide

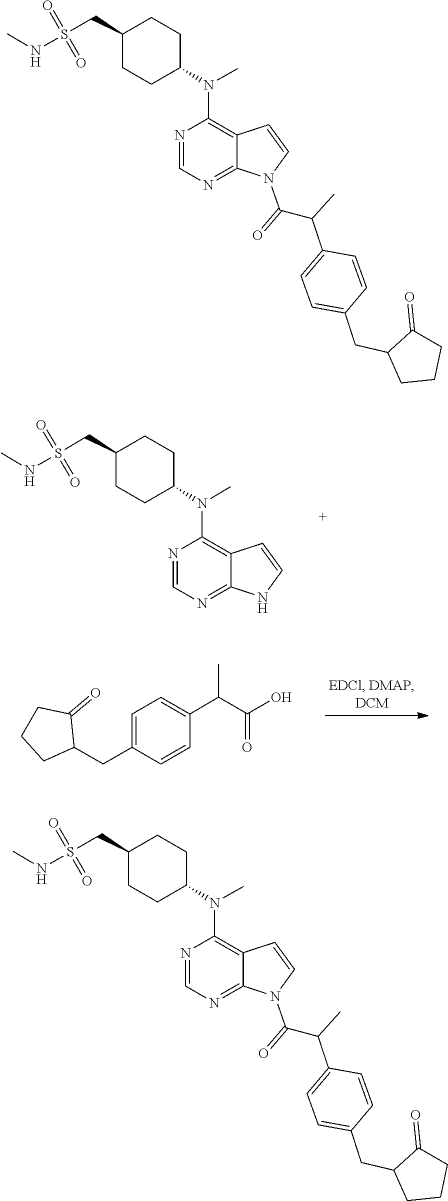 [SMILES: CNS(=O)(=O)C[C@H]1CC[C@H](N(C)c2ncnc3c2ccn3C(=O)C(C)c2ccc(CC3CCCC3=O)cc2)CC1 ; CNS(=O)(=O)C[C@H]1CC[C@H](N(C)c2ncnc3[nH]ccc23)CC1 ; CC(C(=O)O)c1ccc(CC2CCCC2=O)cc1 ; CNS(=O)(=O)C[C@H]1CC[C@H](N(C)c2ncnc3c2ccn3C(=O)C(C)c2ccc(CC3CCCC3=O)cc2)CC1]

### Synthesis of N-methyl-1-((trans)-4-(methyl (7-(2-(4-((2-oxocyclopentyl) methyl) phenyl) propanoyl)-7H-pyrrolo [2, 3-d] pyrimidin-4-yl) amino) cyclohexyl) methanesulfonamide

N-methyl-1-((trans)-4-(methyl (7H-pyrrolo [2, 3-d] pyrimidin-4-yl) amino) cyclohexyl) methanesulfonamide (oclacitinib, 168 mg, 0.5 mmol), 4-dimethylamino pyridine (DMAP, 61 mg, 0.5 mmol), 2-[4-(2-oxocyclopentan-1-ylmethyl) phenyl] propanoic acid (loxoprofen, 160 mg, 0.65 mmol), and 1-(3-dimethylaminopropyl)-3-ethylcarbodiimide hydrochloride (EDCI, 144 mg, 0.75 mmol) were dissolved in dichloromethane (15 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:1) to give the title compound as a white solid, 0.2 g, 70.7% yield. MS (m/z): [M+H]⁺ calcd for C₃₀H₃₉N₅O₄S, 566.27; found, 566.4. ¹H NMR (400 MHz, DMSO-d₆) δ 8.36 (s, 1H), 7.65 (d, J=4.2 Hz, 1H), 7.29 (d, J=7.8 Hz, 2H), 7.09 (d, J=8.1 Hz, 2H), 6.95-6.79 (m, 2H), 6.09 (q, J=6.9 Hz, 1H), 4.63 (s, 1H), 3.14 (s, 3H), 2.98-2.87 (m, 3H), 2.58 (d, J=2.6 Hz, 3H), 2.41-2.17 (m, 3H), 2.10-2.00 (m, 3H), 1.92-1.77 (m, 3H), 1.75-1.60 (m, 5H), 1.52 (d, J=7.0 Hz, 3H), 1.46-1.38 (m, 1H), 1.35-1.22 (m, 2H).

### Example 45

### (3R)-3-cyclopentyl-3-(4-(7-(2-(1, 8-diethyl-1, 3, 4, 9-tetrahydropyran [3, 4-b] indol-1-yl) acetyl)-7H-pyrrolo [2, 3-d] pyrimidin-4-yl)-1H-pyrazol-1-yl) propanenitrile

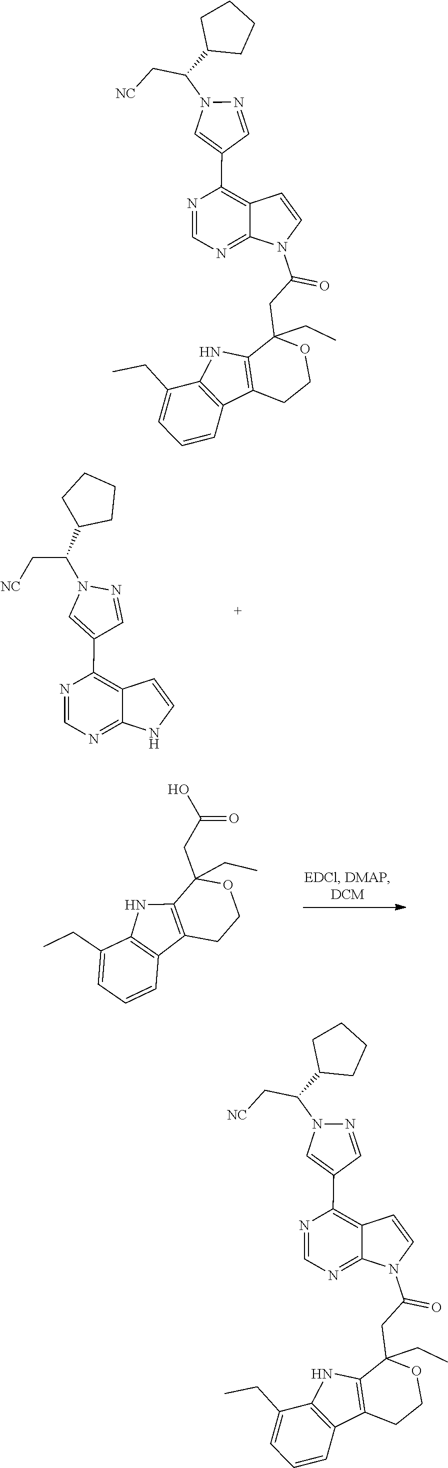 [SMILES: CCc1cccc2c3c([nH]c12)C(CC)(CC(=O)n1ccc2c(-c4cnn([C@H](CC#N)C5CCCC5)c4)ncnc21)OCC3 ; N#CC[C@H](C1CCCC1)n1cc(-c2ncnc3[nH]ccc23)cn1 ; CCc1cccc2c3c([nH]c12)C(CC)(CC(=O)O)OCC3 ; CCc1cccc2c3c([nH]c12)C(CC)(CC(=O)n1ccc2c(-c4cnn([C@H](CC#N)C5CCCC5)c4)ncnc21)OCC3]

### Synthesis of (3R)-3-cyclopentyl-3-(4-(7-(2-(1, 8-diethyl-1, 3, 4, 9-tetrahydropyran [3, 4-b] indol-1-yl) acetyl)-7H-pyrrolo [2, 3-d] pyrimidin-4-yl)-1H-pyrazol-1-yl) propanenitrile

(R)-3-(4-(7h-pyrrolo [2, 3-d] pyridin-4-yl)-1h-pyrazol-1-yl)-3-cyclopentylpropane (ruxolitinib, 184 mg, 0.6 mmol), 4-dimethylamino pyridine (DMAP, 7 mg, 0.06 mmol), 1, 8-diethyl-1, 3, 4, 9-tetrahydropyran [3, 4-b] indole-1-acetic acid (etodolac, 224 mg, 0.78 mmol), and 1-(3-dimethylaminopropyl)-3-ethylcarbodiimide hydrochloride (EDCI, 173 mg, 0.9 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:1) to give the title compound as a white solid, 0.28 g, 81.5% yield. MS (m/z): [M+H]⁺ calcd for C₃₄H₃₇N₇O₂, 576.30; found, 576.2. ¹H NMR (400 MHz, DMSO-d₆) δ 10.54 (s, 1H), 8.88 (d, J=3.1 Hz, 2H), 8.42 (s, 1H), 8.09 (d, J=4.2 Hz, 1H), 7.30 (d, J=4.3 Hz, 1H), 7.24 (dd, J=7.4, 1.5 Hz, 1H), 6.96-6.87 (m, 2H), 4.66-4.51 (m, 2H), 4.19 (dd, J=14.5, 1.7 Hz, 1H), 3.88 (ddd, J=11.8, 7.2, 5.0 Hz, 1H), 3.76 (dt, J=11.1, 4.6 Hz, 1H), 3.30-3.15 (m, 2H), 2.86 (q, J=7.5 Hz, 2H), 2.64-2.56 (m, 2H), 2.44 (q, J=8.4 Hz, 1H), 2.18 (q, J=7.2 Hz, 2H), 1.89-1.78 (m, 1H), 1.70-1.41 (m, 4H), 1.41-1.22 (m, 6H), 0.73 (t, J=7.3 Hz, 3H).

### Example 46

### tert-butyl ((S)-2-(4-(methyl ((trans)-4-((N-methylsulfamoyl) methyl) cyclohexyl) amino)-7H-pyrrolo [2, 3-d] pyrimidin-7-yl)-2-oxo-1-phenylethyl) carbamate

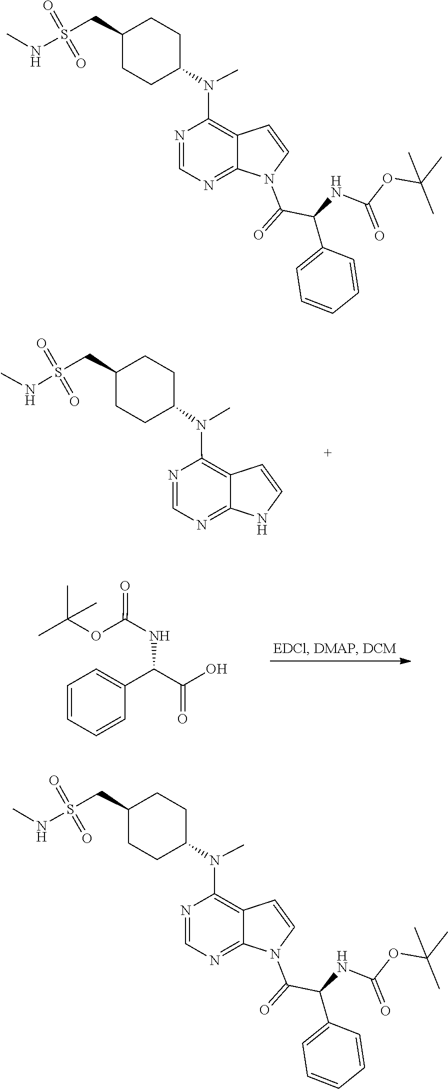

### Synthesis of tert-butyl ((S)-2-(4-(methyl ((trans)-4-((N-methylsulfamoyl) methyl) cyclohexyl) amino)-7H-pyrrolo [2, 3-d] pyrimidin-7-yl)-2-oxo-1-phenylethyl) carbamate

N-methyl-1-((trans)-4-(methyl (7H-pyrrolo [[2, 3-d] pyrimidin-4-yl) amino) cyclohexyl) methane sulfonamide (oclacitinib, 168 mg, 0.5 mmol), 4-dimethylamino pyridine (DMAP, 61 mg, 0.5 mmol), N-Boc-L-phenylglycine (163 mg, 0.65 mmol) and 1-(3-dimethylaminopropyl)-3-ethylcarbodiimide hydrochloride (EDCI, 144 mg, 0.75 mmol) were dissolved in dichloromethane (15 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:1) to give the title compound as a white solid, 0.027 g, 9.4% yield. MS (m/z): [M+H]⁺ calcd for C₂₈H₃₈N₆O₅S, 571.26; found, 571.3. ¹H NMR (400 MHz, DMSO-d6) δ 8.36 (s, 1H), 7.80 (d, J=7.7 Hz, 1H), 7.68 (d, J=4.1 Hz, 1H), 7.56-7.45 (m, 3H), 7.29 (d, J=7.7 Hz, 3H), 6.86 (d, J=4.9 Hz, 2H), 4.63 (s, 1H), 3.14 (s, 3H), 2.94 (d, J=6.2 Hz, 2H), 2.58 (d, J=5.0 Hz, 3H), 2.03 (d, J=12.7 Hz, 2H), 1.90-1.79 (m, 1H), 1.67 (d, J=8.0 Hz, 4H), 1.39 (s, 9H), 1.32-1.21 (m, 2H).

### Example 47

### 2-(3-(4-(7-(2-((2, 3-Dimethylphenyl) amino) benzoyl)-7H-pyrrolo [2, 3-d] pyrimidin-4-yl)-1H-pyrazol-1-yl)-1-(ethylsulfonyl) azetidin-3-yl) acetonitrile

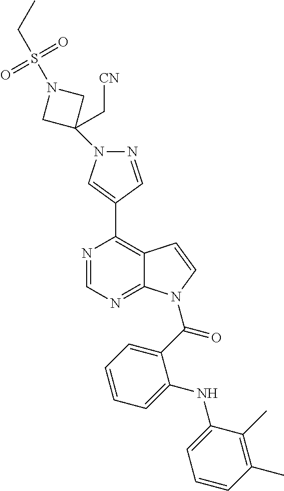 [SMILES: CCS(=O)(=O)N1CC(CC#N)(n2cc(-c3ncnc4c3ccn4C(=O)c3ccccc3Nc3cccc(C)c3C)cn2)C1]

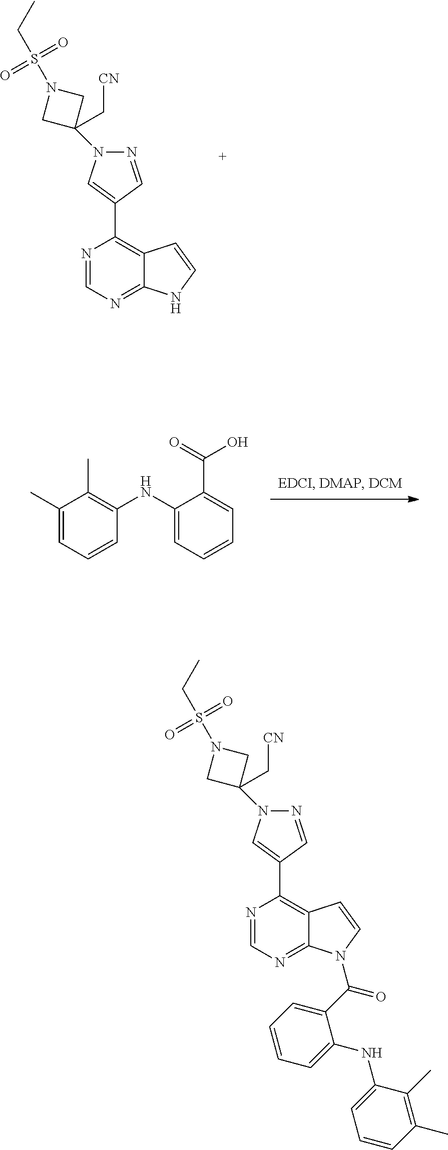 [SMILES: CCS(=O)(=O)N1CC(CC#N)(n2cc(-c3ncnc4[nH]ccc34)cn2)C1 ; Cc1cccc(Nc2ccccc2C(=O)O)c1C ; CCS(=O)(=O)N1CC(CC#N)(n2cc(-c3ncnc4c3ccn4C(=O)c3ccccc3Nc3cccc(C)c3C)cn2)C1]

### Synthesis of 2-(3-(4-(7-(2-((2, 3-dimethylphenyl) amino) benzoyl)-7H-pyrrolo [2, 3-d] pyrimidin-4-yl)-1H-pyrazol-1-yl)-1-(ethylsulfonyl) azetidin-3-yl) acetonitrile

1-(Ethylsulfonyl)-3-[4-(7H-pyrrolo [2, 3-d] pyrimidin-4-yl)-1H-pyrazol-1-yl]-3-azetidine acetonitrile (baricitinib, 371 mg, 1 mmol), 4-dimethylamino pyridine (DMAP, 12 mg, 0.1 mmol), 2-((2, 3-dimethylphenyl) amino) benzoic acid (mefenamic acid, 289 mg, 1.2 mmol), and 1-(3-dimethylaminopropyl)-3-ethylcarbodiimide hydrochloride (EDCI, 289 mg, 1.5 mmol) were dissolved in dichloromethane (10 mL) and stirred at room temperature for 24 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further purified by silica gel column chromatography (petroleum ether/ethyl acetate=10:1 to 1:1) to give the title compound as a yellow solid, 0.25 g, 42% yield. MS (m/z): [M+H]⁺ calcd for C₃₁H₃₀N₈O₃S, 595.22; found, 595.2. ¹H NMR (400 MHz, DMSO-d₆) δ 9.01 (s, 1H), 8.76 (s, 1H), 8.53 (s, 1H), 8.44 (s, 1H), 7.90 (d, J=4.1 Hz, 1H), 7.42 (ddd, J=12.4, 6.6, 3.0 Hz, 3H), 7.08-6.99 (m, 2H), 6.94 (dd, J=5.6, 3.2 Hz, 1H), 6.91-6.75 (m, 2H), 4.62 (d, J=9.1 Hz, 2H), 4.26 (d, J=9.1 Hz, 2H), 3.71 (s, 2H), 3.24 (q, J=7.3 Hz, 2H), 2.23 (s, 3H), 1.97 (s, 3H), 1.25 (t, J=7.5 Hz, 3H).

### Example 48

### 4-(((3R, 4R)-1-(2-cyanoacetyl)-4-methylpiperidin-3-yl) (methyl) amino)-7H-pyrrolo [2, 3-d] pyrimidin-7-yl) methyl (S)-2-(6-methoxynaphthalen-2-yl) propanoate

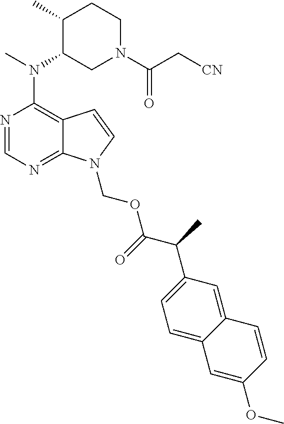

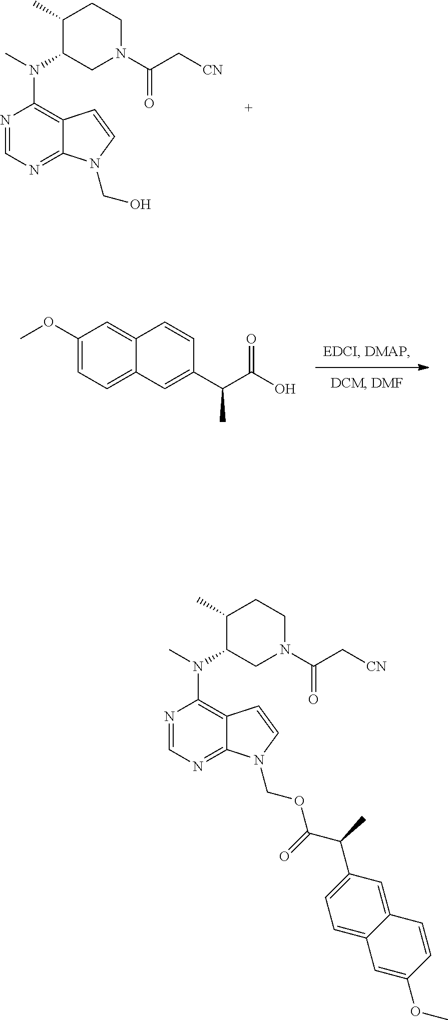

### Synthesis of 4-(((3R, 4R)-1-(2-cyanoacetyl)-4-methylpiperidin-3-yl) (methyl) amino)-7H-pyrrolo [2, 3-d] pyrimidin-7-yl) methyl (S)-2-(6-methoxynaphthalen-2-yl) propanoate

3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile (170 mg, 0.5 mmol), 4-dimethylamino pyridine (DMAP, 61 mg, 0.5 mmol), (S)-(+)-2-(6-methoxy-2-naphthyl) propanoic acid (naproxen, 170 mg, 0.75 mmol) and 1-(3-dimethylaminopropyl)-3-ethylcarbodiimide hydrochloride (EDCI, 144 mg, 0.75 mmol) were dissolved in a mixed solvent of dichloromethane (8.5 mL) and dimethylformamide (0.3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:1) to give the title compound as a white solid, 0.107 g, 38.6% yield. MS (m/z): [M+H]⁺ calcd for C₃₁H₃₄N₆O₄, 555.26; found, 555.3.1H NMR (400 MHz, Chloroform-d) δ 8.31 (d, J=4.1 Hz, 1H), 7.69-7.49 (m, 3H), 7.32 (dt, J=8.6, 2.5 Hz, 1H), 7.20-7.02 (m, 3H), 6.47 (d, J=3.8 Hz, 1H), 6.22-6.06 (m, 2H), 5.10 (d, J=8.2 Hz, 1H), 4.06 (dd, J=13.4, 4.5 Hz, 1H), 3.91 (s, 3H), 3.88-3.66 (m, 2H), 3.66-3.44 (m, 4H), 3.34 (d, J=11.5 Hz, 3H), 2.49 (dt, J=20.2, 6.2 Hz, 1H), 1.99-1.83 (m, 1H), 1.81-1.65 (m, 1H), 1.54 (d, J=7.1 Hz, 3H), 1.08 (dd, J=9.5, 7.0 Hz, 3H).

### Example 49

### (4-(((3R, 4R)-1-(2-cyanoacetyl)-4-methylpiperidin-3-yl) (methyl) amino)-7H-pyrrolo [2, 3-d] pyrimidin-7-yl) methyl 2-(3-benzoylphenyl) propanoate

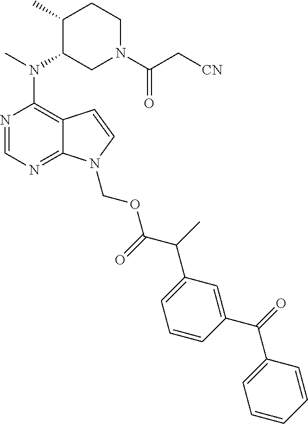 [SMILES: CC(C(=O)OCn1ccc2c(N(C)[C@H]3CN(C(=O)CC#N)CC[C@H]3C)ncnc21)c1cccc(C(=O)c2ccccc2)c1]

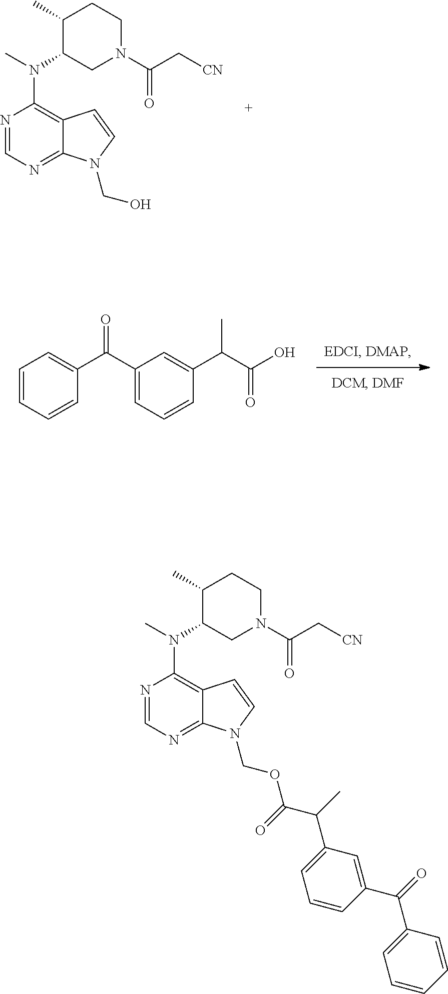 [SMILES: C[C@@H]1CCN(C(=O)CC#N)C[C@@H]1N(C)c1ncnc2c1ccn2CO ; CC(C(=O)O)c1cccc(C(=O)c2ccccc2)c1 ; CC(C(=O)OCn1ccc2c(N(C)[C@H]3CN(C(=O)CC#N)CC[C@H]3C)ncnc21)c1cccc(C(=O)c2ccccc2)c1]

### Synthesis of (4-(((3R, 4R)-1-(2-cyanoacetyl)-4-methylpiperidin-3-yl) (methyl) amino)-7H-pyrrolo [2, 3-d] pyrimidin-7-yl) methyl 2-(3-benzoylphenyl) propanoate

3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile (170 mg, 0.5 mmol), 4-dimethylamino pyridine (DMAP, 61 mg, 0.5 mmol), 2-(3-benzoylphenyl) propanoic acid (ketoprofen, 191 mg, 1.5 mmol) and 1-(3-dimethylaminopropyl)-3-ethylcarbodiimide hydrochloride (EDCI, 144 mg, 0.75 mmol) was dissolved in a mixed solvent of dichloromethane (8.5 mL) and dimethylformamide (0.3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:1) to give the title compound as a white solid, 0.153 g, 52.9% yield. MS (m/z): [M+H]⁺ calcd for C₃₃H₃₄N₆O₄, 579.26; found, 579.3. ¹H NMR (400 MHz, Chloroform-d) δ 8.29 (d, J=4.5 Hz, 1H), 7.78-7.69 (m, 2H), 7.68-7.54 (m, 3H), 7.51-7.42 (m, 3H), 7.36 (td, J=7.6, 2.1 Hz, 1H), 7.10-7.01 (m, 1H), 6.52-6.42 (m, 1H), 6.21-6.10 (m, 2H), 5.10 (dt, J=9.0, 4.7 Hz, 1H), 4.09-3.72 (m, 3H), 3.67-3.43 (m, 4H), 3.35 (dd, J=13.1, 4.9 Hz, 3H), 2.56-2.41 (m, 1H), 2.00-1.85 (m, 1H), 1.80-1.71 (m, 1H), 1.50 (d, J=7.2 Hz, 3H), 1.07 (dd, J=10.5, 7.1 Hz, 3H).

### Example 50

### (4-(((3R, 4R)-1-(2-cyanoacetyl)-4-methylpiperidin-3-yl) (methyl) amino)-7H-pyrrolo [2, 3-d] pyrimidin-7-yl) methyl 2-(2-fluoro-[1, 1′-biphenyl]-4-yl) propanoate

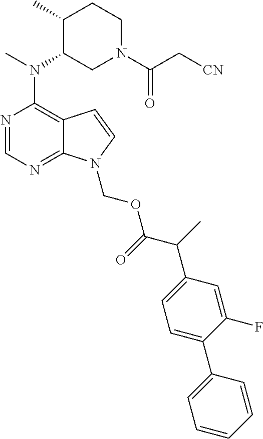 [SMILES: CC(C(=O)OCn1ccc2c(N(C)[C@H]3CN(C(=O)CC#N)CC[C@H]3C)ncnc21)c1ccc(-c2ccccc2)c(F)c1]

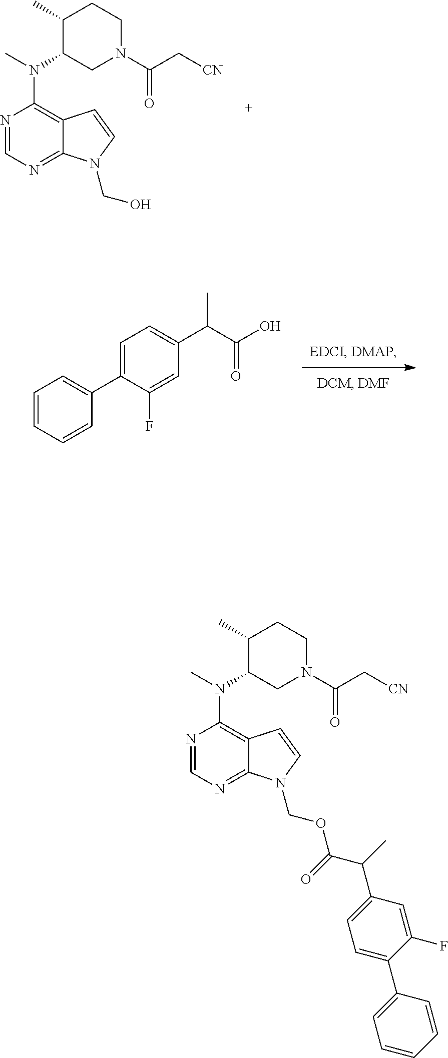 [SMILES: C[C@@H]1CCN(C(=O)CC#N)C[C@@H]1N(C)c1ncnc2c1ccn2CO ; CC(C(=O)O)c1ccc(-c2ccccc2)c(F)c1 ; CC(C(=O)OCn1ccc2c(N(C)[C@H]3CN(C(=O)CC#N)CC[C@H]3C)ncnc21)c1ccc(-c2ccccc2)c(F)c1]

### Synthesis of (4-(((3R, 4R)-1-(2-cyanoacetyl)-4-methylpiperidin-3-yl) (methyl) amino)-7H-pyrrolo [2, 3-d] pyrimidin-7-yl) methyl 2-(2-fluoro-[1, 1′-biphenyl]-4-yl) propanoate

3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile (170 mg, 0.5 mmol), 4-dimethylamino pyridine (DMAP, 61 mg, 0.5 mmol), 2-(2-fluoro-4-biphenyl) propanoic acid (flurbiprofen, 183 mg, 0.75 mmol) and 1-(3-dimethylaminopropyl)-3-ethylcarbodiimide hydrochloride (EDCI, 144 mg, 0.75 mmol) was dissolved in dichloromethane (8.5 mL) and dimethylformamide (0.3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:1) to give the title compound as a white solid, 0.083 g, 29.2% yield. MS (m/z): [M+H]⁺ calcd for C₃₂H₃₃FN₆O₃, 569.26; found, 569.3. ¹H NMR (400 MHz, Chloroform-d) δ 8.31 (d, J=2.2 Hz, 1H), 7.53-7.47 (m, 2H), 7.46-7.40 (m, 2H), 7.39-7.28 (m, 2H), 7.16-7.09 (m, 1H), 7.08-6.85 (m, 2H), 6.51 (q, J=3.3 Hz, 1H), 6.23-6.10 (m, 2H), 5.09 (s, 1H), 4.08-3.69 (m, 4H), 3.67-3.33 (m, 6H), 2.55-2.37 (m, 1H), 1.99-1.81 (m, 1H), 1.80-1.71 (m, 1H), 1.49 (dd, J=7.3, 2.6 Hz, 3H), 1.10-1.00 (m, 3H).

### Example 51

### (4-(((3R, 4R)-1-(2-cyanoacetyl)-4-methylpiperidin-3-yl) (methyl) amino)-7H-pyrrolo [2, 3-d] pyrimidin-7-yl) methyl 2-(2-((2, 6-dichlorophenyl) amino) phenyl) acetate

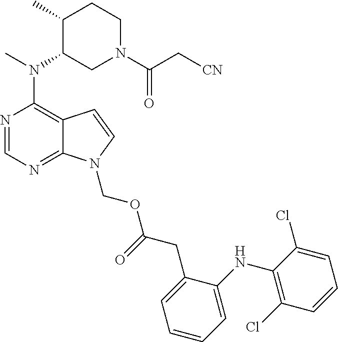 [SMILES: C[C@@H]1CCN(C(=O)CC#N)C[C@@H]1N(C)c1ncnc2c1ccn2COC(=O)Cc1ccccc1Nc1c(Cl)cccc1Cl]

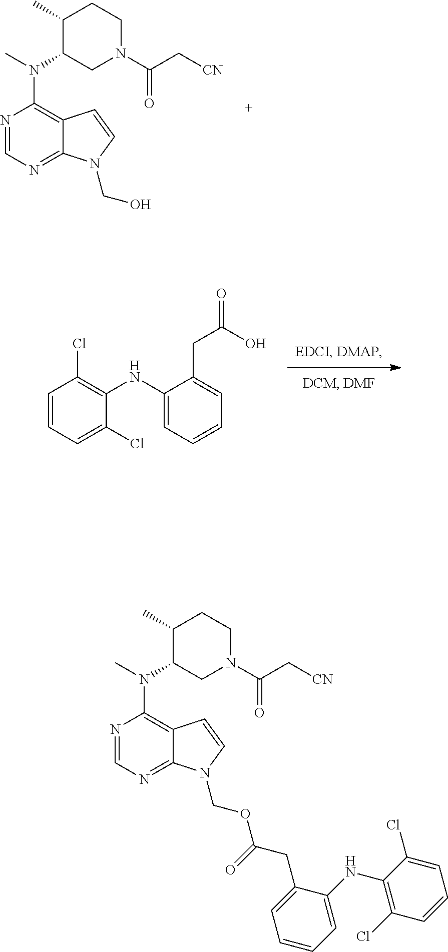 [SMILES: C[C@@H]1CCN(C(=O)CC#N)C[C@@H]1N(C)c1ncnc2c1ccn2CO ; O=C(O)Cc1ccccc1Nc1c(Cl)cccc1Cl ; C[C@@H]1CCN(C(=O)CC#N)C[C@@H]1N(C)c1ncnc2c1ccn2COC(=O)Cc1ccccc1Nc1c(Cl)cccc1Cl]

### Synthesis of (4-(((3R, 4R)-1-(2-cyanoacetyl)-4-methylpiperidin-3-yl) (methyl) amino)-7H-pyrrolo [2, 3-d] pyrimidin-7-yl) methyl 2-(2-((2, 6-dichlorophenyl) amino) phenyl) acetate

3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile (170 mg, 0.5 mmol), 4-dimethylamino pyridine (DMAP, 61 mg, 0.5 mmol), 2-(2, 6-dichloroanilino) phenylacetic acid (diclofenac, 222 mg, 0.75 mmol) and 1-(3-dimethylaminopropyl)-3-ethylcarbodiimide hydrochloride (EDCI, 144 mg, 0.75 mmol) was dissolved in dichloromethane (8.5 mL) and dimethylformamide (0.3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:1) to give the title compound as a white solid, 0.106 g, 34.2% yield. MS (m/z): [M+H]⁺ calcd for C₃₁H₃₁Cl₂N₇O₃, 620.19; found, 620.3. ¹H NMR (400 MHz, Chloroform-d) δ 8.34 (d, J=8.2 Hz, 1H), 7.32 (dd, J=8.1, 1.8 Hz, 2H), 7.18 (dd, J=7.5, 1.7 Hz, 1H), 7.12 (dd, J=9.3, 5.7 Hz, 2H), 6.96 (dt, J=15.1, 7.8 Hz, 2H), 6.64 (d, J=9.4 Hz, 1H), 6.53 (dq, J=7.6, 3.3 Hz, 2H), 6.23 (d, J=3.9 Hz, 2H), 5.13 (s, 1H), 4.10-3.56 (m, 6H), 3.52 (t, J=7.6 Hz, 2H), 3.39 (s, 1H), 3.34 (s, 2H), 2.50 (ddd, J=19.4, 9.7, 4.0 Hz, 1H), 1.99-1.83 (m, 1H), 1.76 (dq, J=17.7, 3.9, 3.2 Hz, 1H), 1.09 (dd, J=13.9, 7.1 Hz, 3H).

### Example 52

### (4-(((3R, 4R)-1-(2-cyanoacetyl)-4-methylpiperidin-3-yl) (methyl) amino)-7H-pyrrolo [2, 3-d] pyrimidin-7-yl) methyl 2-(3-phenoxyphenyl) propanoate

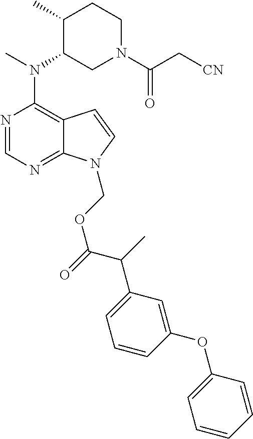 [SMILES: CC(C(=O)OCn1ccc2c(N(C)[C@H]3CN(C(=O)CC#N)CC[C@H]3C)ncnc21)c1cccc(Oc2ccccc2)c1]

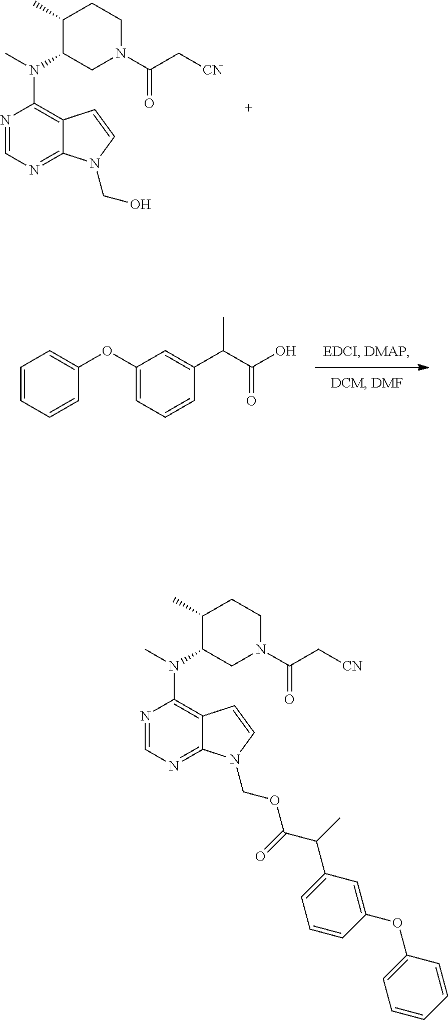 [SMILES: C[C@@H]1CCN(C(=O)CC#N)C[C@@H]1N(C)c1ncnc2c1ccn2CO ; CC(C(=O)O)c1cccc(Oc2ccccc2)c1 ; CC(C(=O)OCn1ccc2c(N(C)[C@H]3CN(C(=O)CC#N)CC[C@H]3C)ncnc21)c1cccc(Oc2ccccc2)c1]

### Synthesis of (4-(((3R, 4R)-1-(2-cyanoacetyl)-4-methylpiperidin-3-yl) (methyl) amino)-7H-pyrrolo [2, 3-d] pyrimidin-7-yl) methyl 2-(3-phenoxyphenyl) propanoate

3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile (170 mg, 0.5 mmol), 4-dimethylamino pyridine (DMAP, 61 mg, 0.5 mmol), 2-(3-phenoxyphenyl) propanoic acid (fenoprofen, 182 mg, 0.75 mmol) and 1-(3-dimethylaminopropyl)-3-ethylcarbodiimide hydrochloride (EDCI, 144 mg, 0.75 mmol) was dissolved in dichloromethane (8.5 mL) and dimethylformamide (0.3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:1) to give the title compound as a white solid, 0.13 g, 45.9% yield. MS (m/z): [M+H]⁺ calcd for C₃₂H₃₄N₆O₄, 567.26; found, 567.3. ¹H NMR (400 MHz, Chloroform-d) δ 8.32 (d, J=6.6 Hz, 1H), 7.32 (t, J=7.8 Hz, 2H), 7.20 (td, J=7.9, 2.2 Hz, 1H), 7.13-7.05 (m, 2H), 6.96 (d, J=7.2 Hz, 3H), 6.92-6.87 (m, 1H), 6.83 (d, J=8.2 Hz, 1H), 6.51 (t, J=3.1 Hz, 1H), 6.21-6.07 (m, 2H), 5.11 (dp, J=9.4, 4.8, 4.4 Hz, 1H), 4.10-3.65 (m, 3H), 3.63-3.49 (m, 3H), 3.47-3.41 (m, 1H), 3.40-3.31 (m, 3H), 2.48 (dp, J=25.5, 6.1 Hz, 1H), 1.94 (dt, J=9.4, 4.6 Hz, 1H), 1.80-1.66 (m, 1H), 1.45 (d, J=7.2 Hz, 3H), 1.08 (dd, J=12.3, 7.1 Hz, 3H).

### Example 53

### (4-(((3R, 4R)-1-(2-cyanoacetyl)-4-methylpiperidin-3-yl) (methyl) amino)-7H-pyrrolo [2, 3-d] pyrimidin-7-yl) methyl 2-(4-((2-oxocyclopentyl) methyl) phenyl) propanoate

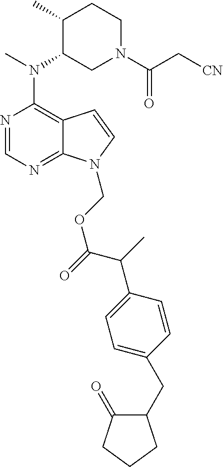 [SMILES: CC(C(=O)OCn1ccc2c(N(C)[C@H]3CN(C(=O)CC#N)CC[C@H]3C)ncnc21)c1ccc(CC2CCCC2=O)cc1]

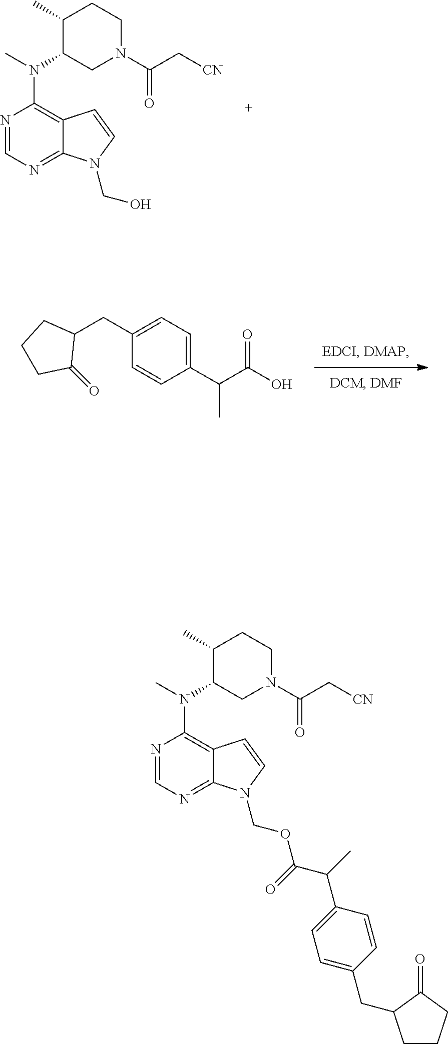 [SMILES: C[C@@H]1CCN(C(=O)CC#N)C[C@@H]1N(C)c1ncnc2c1ccn2CO ; CC(C(=O)O)c1ccc(CC2CCCC2=O)cc1 ; CC(C(=O)OCn1ccc2c(N(C)[C@H]3CN(C(=O)CC#N)CC[C@H]3C)ncnc21)c1ccc(CC2CCCC2=O)cc1]

Synthesis of (4-(((3R, 4R)-1-(2-cyanoacetyl)-4-methylpiperidin-3-yl) (methyl) amino)-7H-pyrrolo [2, 3-d] pyrimidin-7-yl) methyl 2-(4-((2-oxocyclopentyl) methyl) phenyl) propanoate 3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile (170 mg, 0.5 mmol), 4-dimethylamino pyridine (DMAP, 61 mg, 0.5 mmol), 2-[4-(2-oxocyclopentan-1-ylmethyl) phenyl] propanoic acid (loxoprofen, 185 mg, 0.75 mmol) and 1-(3-dimethylaminopropyl)-3-ethylcarbodiimide hydrochloride (EDCI, 144 mg, 0.75 mmol) were dissolved in dichloromethane (8.5 mL) and dimethylformamide (0.3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:1) to give the title compound as a white solid, 0.08 g, 28% yield. MS (m/z): [M+H]⁺ calcd for C₃₂H₃₈N₆O₄, 571.30; found, 571.3. ¹H NMR (400 MHz, Chloroform-d) δ 8.34-8.27 (m, 1H), 7.11 (tt, J=9.3, 7.5, 3.4 Hz, 3H), 7.04 (dt, J=8.3, 2.9 Hz, 2H), 6.51 (s, 1H), 6.17 (dd, J=10.5, 4.6 Hz, 1H), 6.10 (dt, J=10.6, 2.8 Hz, 1H), 5.12 (qd, J=8.6, 5.0 Hz, 1H), 4.11-3.57 (m, 5H), 3.50 (q, J=9.1, 7.3 Hz, 3H), 3.40 (d, J=2.2 Hz, 1H), 3.14-3.02 (m, 1H), 2.49 (ddd, J=23.3, 11.0, 4.9 Hz, 2H), 2.33 (dd, J=17.7, 8.4 Hz, 2H), 2.16-2.01 (m, 2H), 1.95 (tdd, J=11.1, 5.8, 2.8 Hz, 2H), 1.83-1.60 (m, 3H), 1.58-1.47 (m, 1H), 1.44 (dd, J=7.2, 1.8 Hz, 3H), 1.08 (t, J=7.2 Hz, 3H).

### Example 54

### (3R)-3-cyclopentyl-3-(4-(7-(2-(3-phenoxyphenyl) propanoyl)-7H-pyrrolo [2, 3-d] pyrimidin-4-yl)-1H-pyrazol-1-yl) propanenitrile

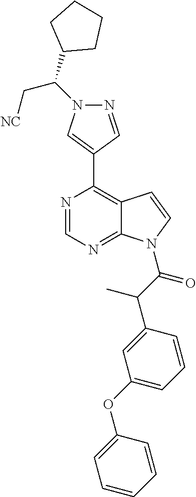 [SMILES: CC(C(=O)n1ccc2c(-c3cnn([C@H](CC#N)C4CCCC4)c3)ncnc21)c1cccc(Oc2ccccc2)c1]

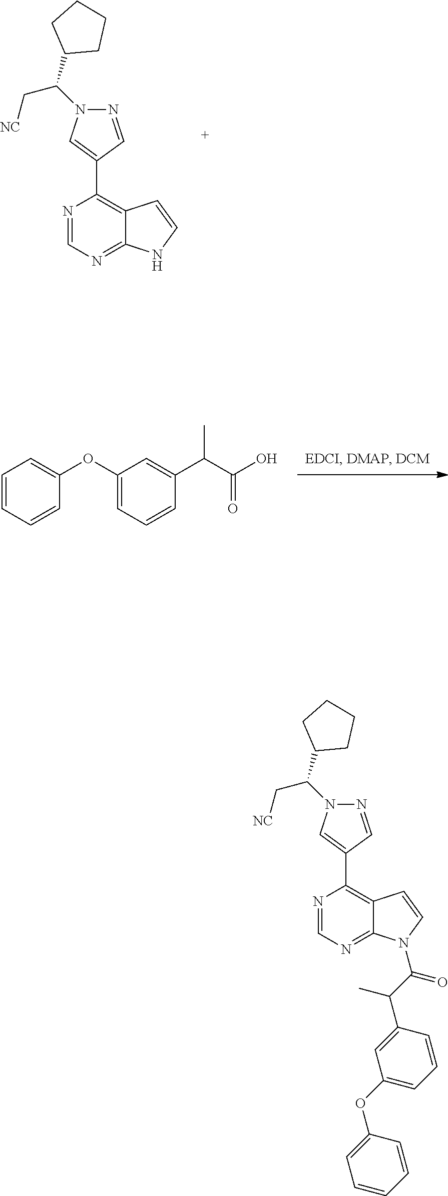 [SMILES: N#CC[C@H](C1CCCC1)n1cc(-c2ncnc3[nH]ccc23)cn1 ; CC(C(=O)O)c1cccc(Oc2ccccc2)c1 ; CC(C(=O)n1ccc2c(-c3cnn([C@H](CC#N)C4CCCC4)c3)ncnc21)c1cccc(Oc2ccccc2)c1]

### Synthesis of (3R)-3-cyclopentyl-3-(4-(7-(2-(3-phenoxyphenyl) propanoyl)-7H-pyrrolo [2, 3-d] pyrimidin-4-yl)-1H-pyrazol-1-yl) propanenitrile

(R)-3-(4-(7h-pyrrolo [2, 3-d] pyridin-4-yl)-1h-pyrazol-1-yl)-3-cyclopentylpropane (Ruxolitinib, 184 mg, 0.6 mmol), 4-dimethylamino pyridine (DMAP, 7 mg, 0.06 mmol), 2-(3-phenoxyphenyl) propanoic acid (fenoprofen, 189 mg, 0.78 mmol) and 1-(3-dimethylaminopropyl)-3-ethylcarbodiimide hydrochloride (EDCI, 173 mg, 0.9 mmol) were dissolved in dichloromethane (10 mL) and stirred at room temperature for 2 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 3:1) to give the title compound as a white solid, 0.2 g, 62.8% yield. MS (m/z): [M+H]⁺ calcd for C₃₂H₃₀N₆O₂, 531.24; found, 531.2. ¹H NMR (400 MHz, DMSO-d₆) δ 8.86 (d, J=13.5 Hz, 2H), 8.41 (s, 1H), 8.09 (d, J=4.2 Hz, 1H), 7.40-7.26 (m, 4H), 7.20-7.09 (m, 2H), 7.07 (t, J=2.1 Hz, 1H), 6.91 (d, J=8.0 Hz, 2H), 6.84 (dd, J=8.1, 2.5 Hz, 1H), 5.96 (q, J=6.9 Hz, 1H), 4.54 (td, J=9.6, 4.2 Hz, 1H), 3.29-3.15 (m, 2H), 2.42 (p, J=8.5 Hz, 1H), 1.82 (dtd, J=12.1, 7.4, 3.8 Hz, 1H), 1.66-1.40 (m, 7H), 1.37-1.18 (m, 3H).

### Example 55

### (4-(((3R, 4R)-1-(2-cyanoacetyl)-4-methylpiperidin-3-yl) (methyl) amino)-7H-pyrrolo [2, 3-d] pyrimidin-7-yl) methyl 2-(1-(4-chlorobenzoyl)-5-methoxy-2-methyl-1H-indol-3-yl) acetate

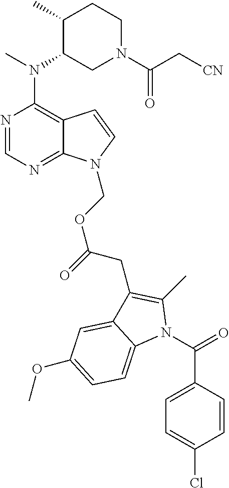 [SMILES: COc1ccc2c(c1)c(CC(=O)OCn1ccc3c(N(C)[C@H]4CN(C(=O)CC#N)CC[C@H]4C)ncnc31)c(C)n2C(=O)c1ccc(Cl)cc1]

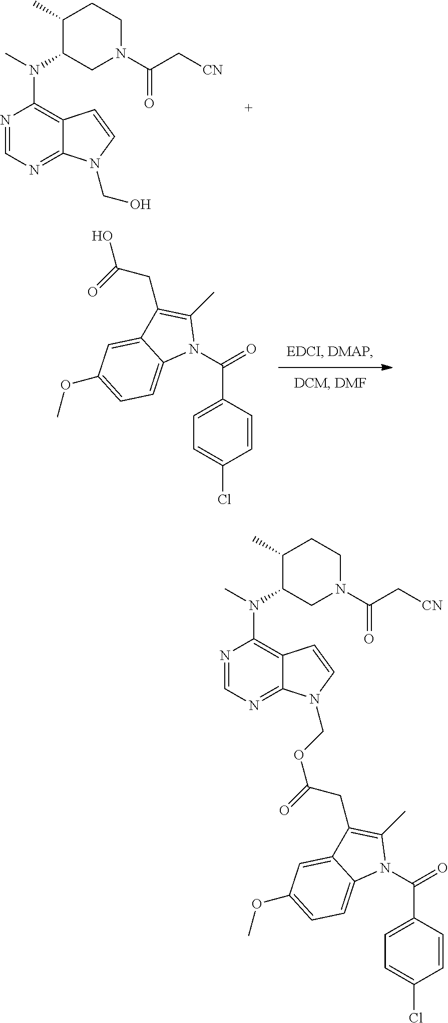

### Synthesis of (4-(((3R, 4R)-1-(2-cyanoacetyl)-4-methylpiperidin-3-yl) (methyl) amino)-7H-pyrrolo [2, 3-d] pyrimidin-7-yl) methyl 2-(1-(4-chlorobenzoyl)-5-methoxy-2-methyl-1H-indol-3-yl) acetate

3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile (170 mg, 0.5 mmol), 4-dimethylamino pyridine (DMAP, 61 mg, 0.5 mmol), 1-(4-chlorobenzoyl)-5-methoxy-2-methyl-1H-indole-3-acetic acid (indomethacin, 277 mg, 0.75 mmol) and 1-(3-dimethylaminopropyl)-3-ethylcarbodiimide hydrochloride (EDCI, 144 mg, 0.75 mmol) were dissolved in dichloromethane (8.5 mL) and dimethylformamide (0.3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further purified by silica gel column chromatography (petroleum ether/ethyl acetate=2:1 to 1:1) to give the title compound as a light yellow solid, 0.036 g, 10.5% yield. MS (m/z): [M+H]⁺ calcd for C₃₆H₃₆ClN₇O₅, 682.25; found, 682.3.1H NMR (400 MHz, Chloroform-d) δ 8.32 (s, 1H), 7.68-7.59 (m, 2H), 7.51-7.43 (m, 2H), 7.09 (d, J=3.8 Hz, 1H), 6.91-6.78 (m, 2H), 6.65 (dd, J=9.0, 2.4 Hz, 1H), 6.50 (dd, J=8.2, 3.8 Hz, 1H), 6.19 (s, 2H), 5.19-5.09 (m, 1H), 4.10-3.73 (m, 6H), 3.70-3.56 (m, 4H), 3.55-3.45 (m, 3H), 3.39 (s, 1H), 2.58-2.42 (m, 1H), 2.29 (d, J=10.3 Hz, 3H), 2.03-1.85 (m, 1H), 1.81-1.73 (m, 1H), 1.09 (dd, J=11.4, 7.1 Hz, 3H).

### Example 56

### (4-(((3R, 4R)-1-(2-cyanoacetyl)-4-methylpiperidin-3-yl) (methyl) amino)-7H-pyrrolo [2, 3-d] pyrimidin-7-yl) methyl 2-acetoxybenzoate

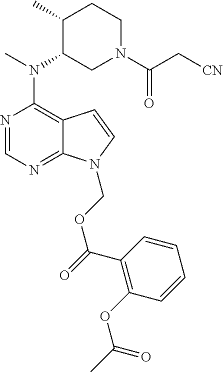 [SMILES: CC(=O)Oc1ccccc1C(=O)OCn1ccc2c(N(C)[C@H]3CN(C(=O)CC#N)CC[C@H]3C)ncnc21]

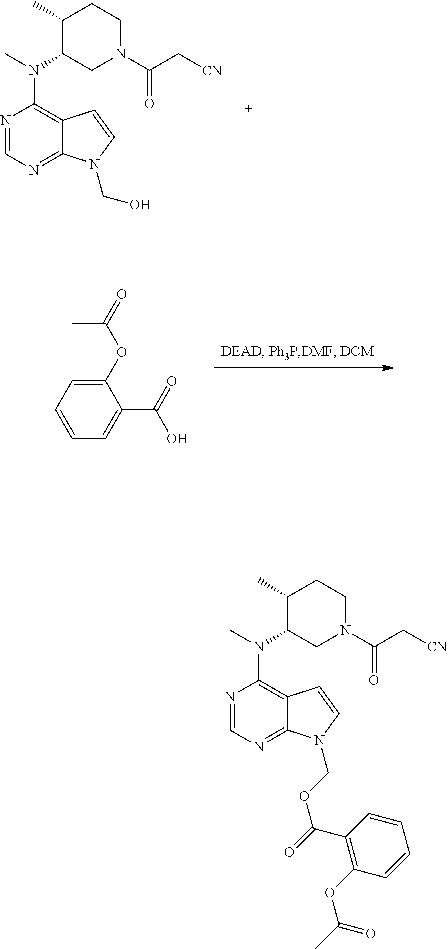 [SMILES: C[C@@H]1CCN(C(=O)CC#N)C[C@@H]1N(C)c1ncnc2c1ccn2CO ; CC(=O)Oc1ccccc1C(=O)O ; CC(=O)Oc1ccccc1C(=O)OCn1ccc2c(N(C)[C@H]3CN(C(=O)CC#N)CC[C@H]3C)ncnc21]

Synthesis of (4-(((3R, 4R)-1-(2-cyanoacetyl)-4-methylpiperidin-3-yl) (methyl) amino)-7H-pyrrolo [2, 3-d] pyrimidin-7-yl) methyl 2-acetoxybenzoate 3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile (170 mg, 0.5 mmol), 4-dimethylamino pyridine (DMAP, 61 mg, 0.5 mmol), 2-acetoxybenzoic acid (aspirin, 135 mg, 0.75 mmol) and 1-(3-dimethylaminopropyl)-3-ethylcarbodiimide hydrochloride (EDCI, 144 mg, 0.75 mmol) was dissolved in dichloromethane (8.5 mL) and dimethylformamide (0.3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further purified by silica gel column chromatography (petroleum ether/ethyl acetate=2:1 to 1:1) to give the title compound as a light yellow solid, 0.178 g, 70.6% yield. MS (m/z): [M+H]⁺ calcd for C₂₆H₂₈N₆O₅, 505.21; found, 505.3. ¹H NMR (400 MHz, Chloroform-d) δ 8.34 (d, J=6.5 Hz, 1H), 8.01 (d, J=7.9 Hz, 1H), 7.60-7.50 (m, 1H), 7.31-7.18 (m, 2H), 7.07 (d, J=8.1 Hz, 1H), 6.57 (d, J=3.8 Hz, 1H), 6.37 (d, J=2.6 Hz, 2H), 5.12 (tt, J=8.3, 4.6 Hz, 1H), 4.25-3.98 (m, 1H), 3.80 (ddt, J=13.2, 8.8, 4.7 Hz, 1H), 3.62-3.50 (m, 3H), 3.37 (d, J=15.2 Hz, 3H), 2.49 (dp, J=24.6, 6.0 Hz, 1H), 2.25 (d, J=12.7 Hz, 3H), 1.93 (dtt, J=17.8, 9.0, 4.5 Hz, 2H), 1.74 (dtt, J=17.9, 7.6, 3.5 Hz, 1H), 1.08 (dd, J=13.2, 7.0 Hz, 3H).

### Example 57

### (R)-3-(4-(7-(2-((3-chloro-2-methylphenyl) amino) benzoyl)-7H-pyrrolo [2, 3-d] pyrimidin-4-yl)-1H-pyrazol-1-yl)-3-cyclopentylpropanenitrile

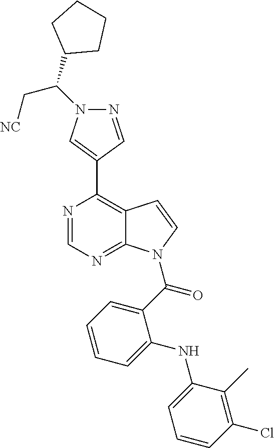 [SMILES: Cc1c(Cl)cccc1Nc1ccccc1C(=O)n1ccc2c(-c3cnn([C@H](CC#N)C4CCCC4)c3)ncnc21]

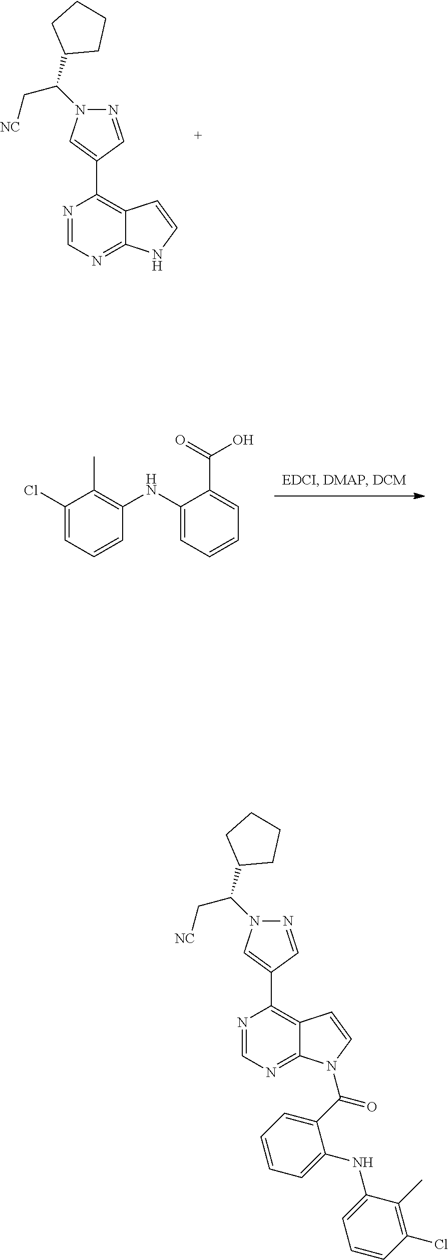 [SMILES: N#CC[C@H](C1CCCC1)n1cc(-c2ncnc3[nH]ccc23)cn1 ; Cc1c(Cl)cccc1Nc1ccccc1C(=O)O ; Cc1c(Cl)cccc1Nc1ccccc1C(=O)n1ccc2c(-c3cnn([C@H](CC#N)C4CCCC4)c3)ncnc21]

### Synthesis of (R)-3-(4-(7-(2-((3-chloro-2-methylphenyl) amino) benzoyl)-7H-pyrrolo [2, 3-d] pyrimidin-4-yl)-1H-pyrazol-1-yl)-3-cyclopentylpropanenitrile

(R)-3-(4-(7h-pyrrolo [2, 3-d] pyridin-4-yl)-1h-pyrazol-1-yl)-3-cyclopentylpropane (Ruxolitinib, 306 mg, 1 mmol), 4-dimethylamino pyridine (DMAP, 12 mg, 0.1 mmol), 2-((3-chloro-2-methylphenyl) amino) benzoic acid (tolfenamic acid, 261 mg, 1 mmol) and 1-(3-dimethylaminopropyl)-3-ethylcarbodiimide hydrochloride (EDCI, 289 mg, 1.5 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further purified by silica gel column chromatography (petroleum ether/ethyl acetate=5:1 to 1:1) to give the title compound as a yellow solid, 0.26 g, 47.3% yield. MS (m/z): [M+H]⁺ calcd for C₃₁H₂₈ClN₇O, 550.20; found, 550.1. ¹H NMR (400 MHz, DMSO-d₆) δ 8.87 (s, 1H), 8.70 (s, 1H), 8.41 (s, 1H), 8.23 (s, 1H), 7.80 (d, J=4.1 Hz, 1H), 7.49 (t, J=7.7 Hz, 2H), 7.27 (d, J=4.1 Hz, 1H), 7.12-7.00 (m, 3H), 7.00-6.91 (m, 2H), 4.55 (td, J=9.6, 4.2 Hz, 1H), 3.29-3.17 (m, 2H), 2.43 (p, J=8.5 Hz, 1H), 2.02 (s, 3H), 1.83 (dtd, J=11.9, 7.3, 3.9 Hz, 1H), 1.69-1.42 (m, 4H), 1.41-1.27 (m, 2H), 1.23 (td, J=9.4, 8.7, 2.9 Hz, 1H).

### Example 58

### N-(4-(2-(4-(methyl ((trans)-4-((N-methylsulfamoyl) methyl) cyclohexyl) amino)-7H-pyrrolo [2, 3-d] pyrimidin-7-yl)-2-oxoethyl) phenyl) acetamide

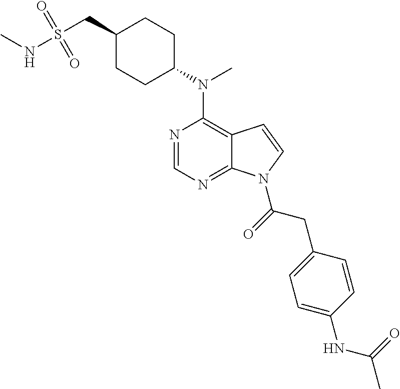 [SMILES: CNS(=O)(=O)C[C@H]1CC[C@H](N(C)c2ncnc3c2ccn3C(=O)Cc2ccc(NC(C)=O)cc2)CC1]

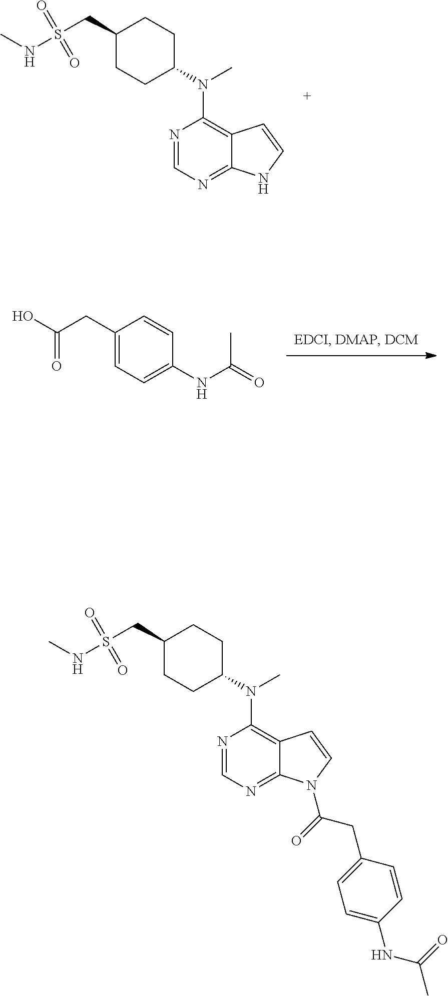 [SMILES: CNS(=O)(=O)C[C@H]1CC[C@H](N(C)c2ncnc3[nH]ccc23)CC1 ; CC(=O)Nc1ccc(CC(=O)O)cc1 ; CNS(=O)(=O)C[C@H]1CC[C@H](N(C)c2ncnc3c2ccn3C(=O)Cc2ccc(NC(C)=O)cc2)CC1]

### Synthesis of N-(4-(2-(4-(methyl ((trans)-4-((N-methylsulfamoyl) methyl) cyclohexyl) amino)-7H-pyrrolo [2, 3-d] pyrimidin-7-yl)-2-oxoethyl) phenyl) acetamide

N-Methyl-1-((trans)-4-(methyl (7H-pyrrolo [2, 3-d] pyrimidin-4-yl) amino) cyclohexyl) methanesulfonamide (oclacitinib, 168 mg, 0.5 mmol), 4-dimethylamino pyridine (DMAP, 61 mg, 0.5 mmol), 2-(4-acetamidophenyl) acetic acid (actarit, 126 mg, 0.65 mmol) and 1-(3-dimethylaminopropyl)-3-ethylcarbodiimide hydrochloride (EDCI, 144 mg, 0.75 mmol) were dissolved in dichloromethane (15 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 0:1) to give the title compound as a white solid, 0.017 g, 6.6% yield. MS (m/z): [M+H]⁺ calcd for C₂₅H₃₂N₆O₄S, 513.22; found, 513.3. ¹H NMR (400 MHz, DMSO-d₆) δ 9.90 (s, 1H), 8.37 (s, 1H), 7.68 (d, J=4.2 Hz, 1H), 7.51 (d, J=8.4 Hz, 2H), 7.25 (d, J=8.4 Hz, 2H), 6.88 (d, J=4.9 Hz, 2H), 4.81 (s, 2H), 4.67 (s, 1H), 3.18 (s, 3H), 2.95 (d, J=6.2 Hz, 2H), 2.59 (d, J=4.9 Hz, 3H), 2.03 (s, 5H), 1.94-1.80 (m, 1H), 1.70 (dd, J=8.6, 3.4 Hz, 4H), 1.37-1.26 (m, 2H).

### Example 59

### (4-(((3R, 4R)-1-(2-cyanoacetyl)-4-methylpiperidin-3-yl) (methyl) amino)-7H-pyrrolo [2, 3-d] pyrimidin-7-yl) methyl 2-(4-acetamidophenyl) acetate

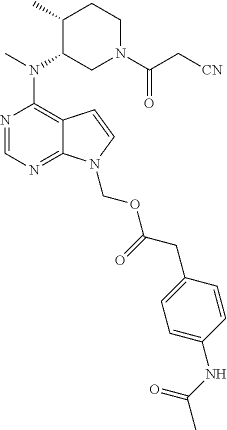 [SMILES: CC(=O)Nc1ccc(CC(=O)OCn2ccc3c(N(C)[C@H]4CN(C(=O)CC#N)CC[C@H]4C)ncnc32)cc1]

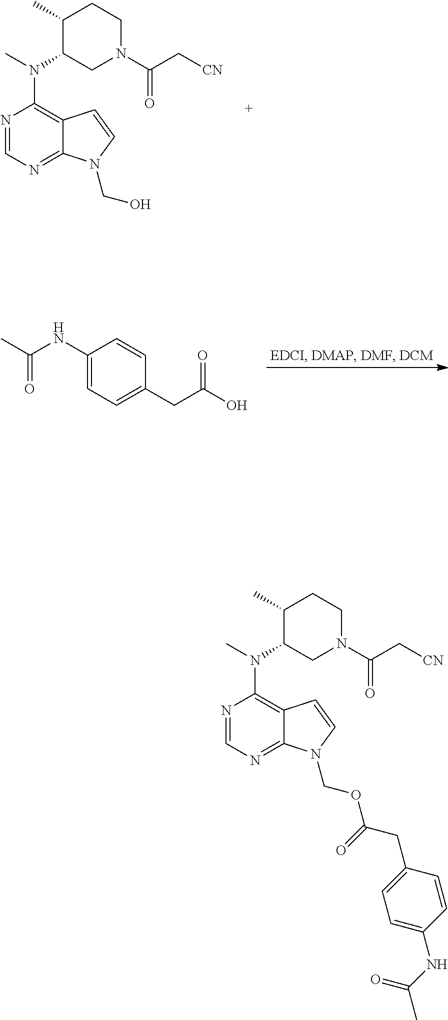 [SMILES: C[C@@H]1CCN(C(=O)CC#N)C[C@@H]1N(C)c1ncnc2c1ccn2CO ; CC(=O)Nc1ccc(CC(=O)O)cc1 ; CC(=O)Nc1ccc(CC(=O)OCn2ccc3c(N(C)[C@H]4CN(C(=O)CC#N)CC[C@H]4C)ncnc32)cc1]

### Synthesis of (4-(((3R, 4R)-1-(2-cyanoacetyl)-4-methylpiperidin-3-yl) (methyl) amino)-7H-pyrrolo [2, 3-d] pyrimidin-7-yl) methyl 2-(4-acetamidophenyl) acetate

3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile (171 mg, 0.5 mmol), 4-dimethylamino pyridine (DMAP, 61 mg, 0.5 mmol), 2-(4-acetamidophenyl) acetic acid (actarit, 145 mg, 0.75 mmol) and 1-(3-dimethylaminopropyl)-3-ethylcarbodiimide hydrochloride (EDCI, 144 mg, 0.75 mmol) was dissolved in a mixed solvent of dichloromethane (8.5 mL) and N, N-dimethylformamide (0.3 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.154 g, yield 59.5%. MS (m/z): [M+H]⁺ calcd for C₂₇H₃₁N₇O₄, 518.24; found, 518.3.1H NMR (400 MHz, Chloroform-d) δ 8.33 (d, J=4.2 Hz, 1H), 7.71 (d, J=29.0 Hz, 1H), 7.40 (d, J=8.1 Hz, 2H), 7.10 (dt, J=13.1, 4.8 Hz, 3H), 6.51 (dd, J=8.4, 3.9 Hz, 1H), 6.16 (s, 2H), 5.10 (tt, J=9.2, 4.4 Hz, 1H), 4.10-3.75 (m, 2H), 3.63-3.44 (m, 6H), 3.36 (d, J=18.1 Hz, 3H), 2.55-2.44 (m, 1H), 2.00 (s, 3H), 1.97-1.87 (m, 1H), 1.83-1.67 (m, 1H), 1.09 (t, J=7.5 Hz, 3H).

### Example 60

### Methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) (S)-2-(4-isobutylphenyl) propanoate

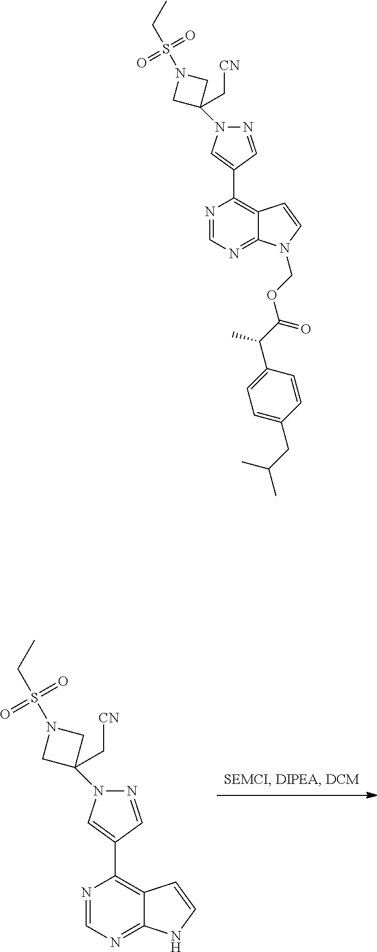 [SMILES: CCS(=O)(=O)N1CC(CC#N)(n2cc(-c3ncnc4c3ccn4COC(=O)[C@@H](C)c3ccc(CC(C)C)cc3)cn2)C1 ; CCS(=O)(=O)N1CC(CC#N)(n2cc(-c3ncnc4[nH]ccc34)cn2)C1]

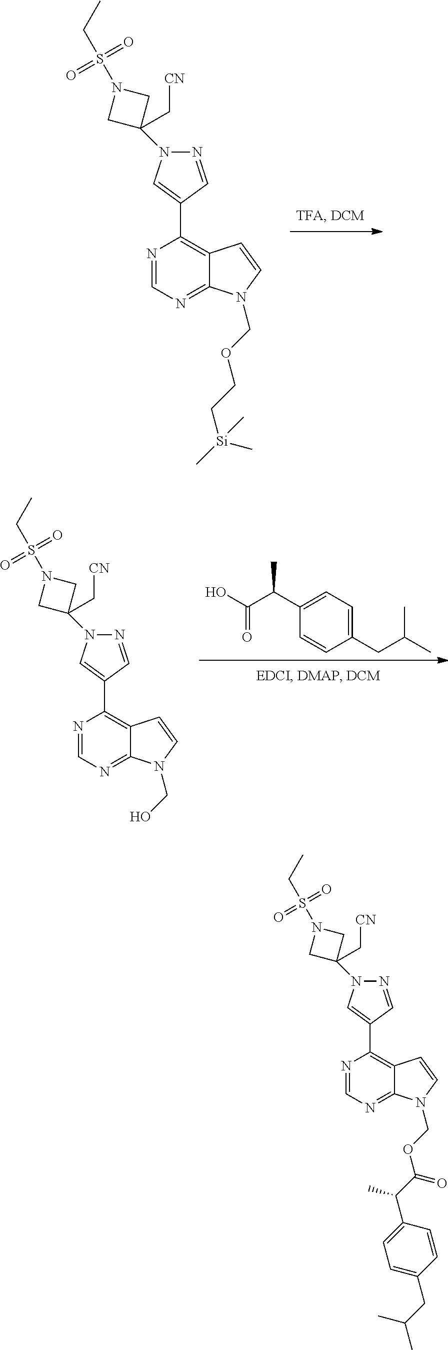 [SMILES: CCS(=O)(=O)N1CC(CC#N)(n2cc(-c3ncnc4c3ccn4COCC[Si](C)(C)C)cn2)C1 ; CCS(=O)(=O)N1CC(CC#N)(n2cc(-c3ncnc4c3ccn4CO)cn2)C1 ; CC(C)Cc1ccc([C@H](C)C(=O)O)cc1 ; CCS(=O)(=O)N1CC(CC#N)(n2cc(-c3ncnc4c3ccn4COC(=O)[C@@H](C)c3ccc(CC(C)C)cc3)cn2)C1]

### First Step: Synthesis of 2-(1-(ethylsulfonyl)-3-(4-(7-((2-(trimethylsilyl) ethoxy) methyl)-7H-pyrrolo [2, 3-d] pyrimidin-4-yl)-1H-pyrazol-1-yl) azetidin-3-yl) acetonitrile

1-(Ethylsulfonyl)-3-[4-(7H-pyrrolo [2, 3-d] pyrimidin-4-yl)-1H-pyrazol-1-yl]-3-azetidine acetonitrile (baricitinib, 7420 mg, 20 mmol) and N, N-diisopropylethyl amine (3.12 g, 24 mmol) were dissolved in dichloromethane (200 mL) under nitrogen protection. After stirring at room temperature for half an hour, (2-(chloromethoxy) ethyl) trimethylsilane (4 g, 24 mmol) was added, stirring was continued at room temperature overnight. The solvent was evaporated under reduced pressure to give the crude product. The crude product was further isolated by silica gel column chromatography to give the title compound as a white solid, 5 g, 49% yield. MS (m/z): [M+H]⁺ calcd for C₂₂H₃₁N₇O₃SSi, 502.20; found, 502.3.

### Step 2: Synthesis of 2-(1-(ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile

Trifluoroacetic acid (6.44 g, 56.5 mmol) was slowly added dropwise to a solution of 2-(1-(ethylsulfonyl)-3-(4-(7-((2-(trimethylsilyl) ethoxy) methyl)-7H-pyrrolo [2, 3-d] pyrimidin-4-yl)-1H-pyrazol-1-yl) azetidin-3-yl) acetonitrile (5 g, 11.3 mmol) in dichloromethane (100 mL) under nitrogen in an ice-water bath. After half an hour, the ice-water bath was removed and the temperature was raised to room temperature and stirring was continued for 24 hours. Saturated sodium bicarbonate solution was added to the above reaction solution at 0° C. to adjust the pH to 8. Then, the mixture was poured into a separation funnel and separated. The organic layer was washed with a saturated salt water solution, and dried over anhydrous sodium sulfate. After filtration, the solvent was evaporated under reduced pressure to give the title product 3.5 g, 90% yield. MS (m/z): [M+H]⁺ calcd for C₁₇H₁₉N₇O₃S, 402.13; found, 402.3.

### Step 3: Synthesis of methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) (S)-2-(4-isobutylphenyl) propanoate

2-(1-(Ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile (201 mg, 0.5 mmol), 4-dimethylamino pyridine (DMAP, 61 mg, 0.5 mmol), (S)-(+)-2-(4-isobutylphenyl) propanoic acid((S)-(+)-ibuprofen, 123.6 mg, 0.6 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:1) to give the title compound as a white solid, 0.025 g, 8.4% yield. MS (m/z): [M+H]⁺ calcd for C₃₀H₃₅N₇O₄S, 590.25, found 590.3.1H NMR (400 MHz, DMSO-d₆) δ 8.95 (s, 1H), 8.77 (s, 1H), 8.48 (s, 1H), 7.70 (d, J=3.8 Hz, 1H), 7.16 (d, J=3.8 Hz, 1H), 7.06 (d, J=7.8 Hz, 2H), 6.96 (d, J=7.8 Hz, 2H), 6.25 (d, J=3.2 Hz, 2H), 4.60 (d, J=9.1 Hz, 2H), 4.25 (d, J=9.1 Hz, 2H), 3.76 (q, J=7.0 Hz, 1H), 3.69 (s, 2H), 3.24 (q, J=7.3 Hz, 2H), 2.32 (d, J=7.1 Hz, 2H), 1.71 (hept, J=6.7 Hz, 1H), 1.34 (d, J=7.1 Hz, 3H), 1.26 (d, J=7.4 Hz, 3H), 0.77 (d, J=6.5 Hz, 6H).

### Example 61

### Methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) (S)-2-(6-methoxynaphthalen-2-yl) propanoate

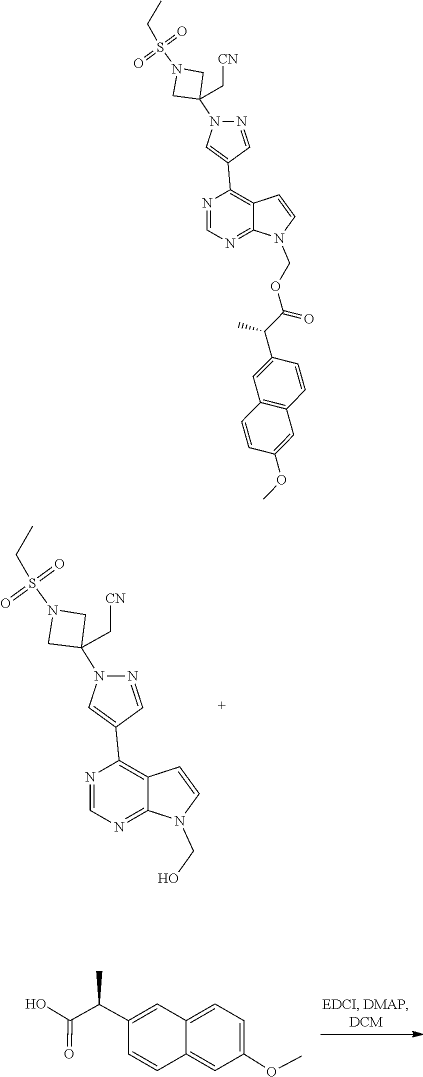 [SMILES: CCS(=O)(=O)N1CC(CC#N)(n2cc(-c3ncnc4c3ccn4COC(=O)[C@@H](C)c3ccc4cc(OC)ccc4c3)cn2)C1 ; CCS(=O)(=O)N1CC(CC#N)(n2cc(-c3ncnc4c3ccn4CO)cn2)C1 ; COc1ccc2cc([C@H](C)C(=O)O)ccc2c1]

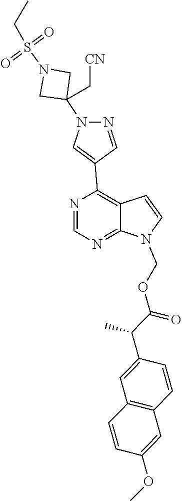 [SMILES: CCS(=O)(=O)N1CC(CC#N)(n2cc(-c3ncnc4c3ccn4COC(=O)[C@@H](C)c3ccc4cc(OC)ccc4c3)cn2)C1]

### Synthesis of methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) (S)-2-(6-methoxynaphthalen-2-yl) propanoate

2-(1-(Ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile (401 mg, 1 mmol), 4-dimethylamino pyridine (DMAP, 61 mg, 0.5 mmol), (S)-(+)-2-(6-methoxy-2-naphthyl) propanoic acid (naproxen, 276 mg, 1.2 mmol) and 1-(3-dimethylaminopropyl)-3-ethylcarbodiimide hydrochloride (EDCI, 288 mg, 1.5 mmol) were dissolved in dichloromethane (10 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=10:1 to 1:1) to give the title compound as a white solid, 0.31 g, 50.5% yield. MS (m/z): [M+H]⁺ calcd for C₃₁H₃₁N₇O₅S, 614.21; found, 614.3. ¹H NMR (400 MHz, DMSO-d₆) δ 8.94 (s, 1H), 8.76 (s, 1H), 8.48 (s, 1H), 7.72 (d, J=3.8 Hz, 1H), 7.65 (dd, J=10.9, 8.8 Hz, 2H), 7.56 (d, J=1.9 Hz, 1H), 7.29 (dd, J=8.5, 1.9 Hz, 1H), 7.22 (d, J=2.6 Hz, 1H), 7.16 (d, J=3.8 Hz, 1H), 7.08 (dd, J=8.9, 2.6 Hz, 1H), 6.28 (s, 2H), 4.61 (d, J=9.1 Hz, 2H), 4.26 (d, J=9.1 Hz, 2H), 3.94 (q, J=7.0 Hz, 1H), 3.83 (s, 3H), 3.70 (s, 2H), 3.24 (q, J=7.3 Hz, 2H), 1.44 (d, J=7.1 Hz, 3H), 1.26 (t, J=7.4 Hz, 3H).

### Example 62

### 4-(1-(3-(cyanomethyl)-1-(ethylsulfonyl) azetidin-3-yl)-1H-pyrazol-4-yl)-7H-pyrrolo [2, 3-d] pyrimidin-7-yl) methyl 2-(3-benzoylphenyl) propanoate

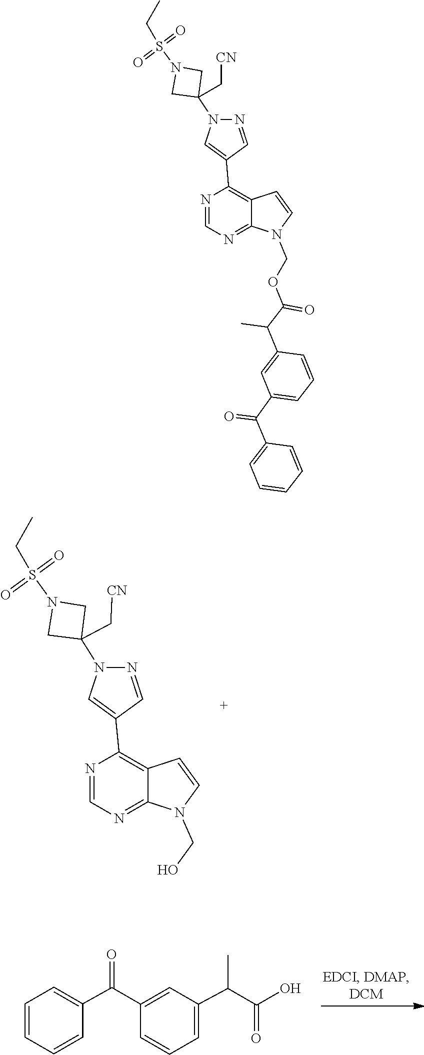 [SMILES: CCS(=O)(=O)N1CC(CC#N)(n2cc(-c3ncnc4c3ccn4COC(=O)C(C)c3cccc(C(=O)c4ccccc4)c3)cn2)C1 ; CCS(=O)(=O)N1CC(CC#N)(n2cc(-c3ncnc4c3ccn4CO)cn2)C1 ; CC(C(=O)O)c1cccc(C(=O)c2ccccc2)c1]

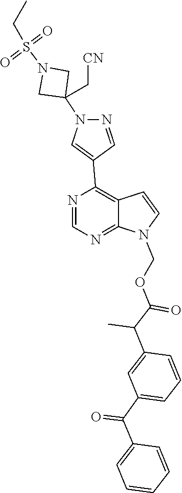 [SMILES: CCS(=O)(=O)N1CC(CC#N)(n2cc(-c3ncnc4c3ccn4COC(=O)C(C)c3cccc(C(=O)c4ccccc4)c3)cn2)C1]

### Synthesis of 4-(1-(3-(cyanomethyl)-1-(ethylsulfonyl) azetidin-3-yl)-1H-pyrazol-4-yl)-7H-pyrrolo [2,3-d] pyrimidin-7-yl) methyl 2-(3-benzoylphenyl) propanoate

2-(1-(Ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile (401 mg, 1 mmol), 4-dimethylamino pyridine (DMAP, 61 mg, 0.5 mmol) 2-(3-benzoylphenyl) propanoic acid (ketoprofen, 305 mg, 1.2 mmol) and 1-(3-dimethylaminopropyl)-3-ethylcarbodiimide hydrochloride (EDCI, 288 mg, 1.5 mmol) were dissolved in dichloromethane (10 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=10:1 to 1:1) to give the title compound as a white solid, 0.43 g, 67.5% yield. MS (m/z): [M+H]⁺ calcd for C₃₃H₃₁N₇O₅S, 638.21; found, 638.2. ¹H NMR (400 MHz, DMSO-d₆) δ 8.95 (s, 1H), 8.75 (s, 1H), 8.48 (s, 1H), 7.71 (d, J=3.8 Hz, 1H), 7.69-7.62 (m, 3H), 7.59 (t, J=1.8 Hz, 1H), 7.57-7.49 (m, 4H), 7.43 (t, J=7.6 Hz, 1H), 7.17 (d, J=3.8 Hz, 1H), 6.28 (d, J=4.4 Hz, 2H), 4.62 (d, J=9.1 Hz, 2H), 4.27 (d, J=9.1 Hz, 2H), 3.99 (q, J=7.1 Hz, 1H), 3.71 (s, 2H), 3.25 (q, J=7.3 Hz, 2H), 1.41 (d, J=7.1 Hz, 3H), 1.26 (t, J=7.3 Hz, 3H).

### Example 63

### (4-(((3R, 4R)-1-(2-cyanoacetyl)-4-methylpiperidin-3-yl) (methyl) amino)-7H-pyrrolo [2, 3-d] pyrimidin-7-yl) methyl 2-(1, 8-diethyl-1, 3, 4, 9-tetrahydropyrano [3, 4-b] indol-1-yl) acetate

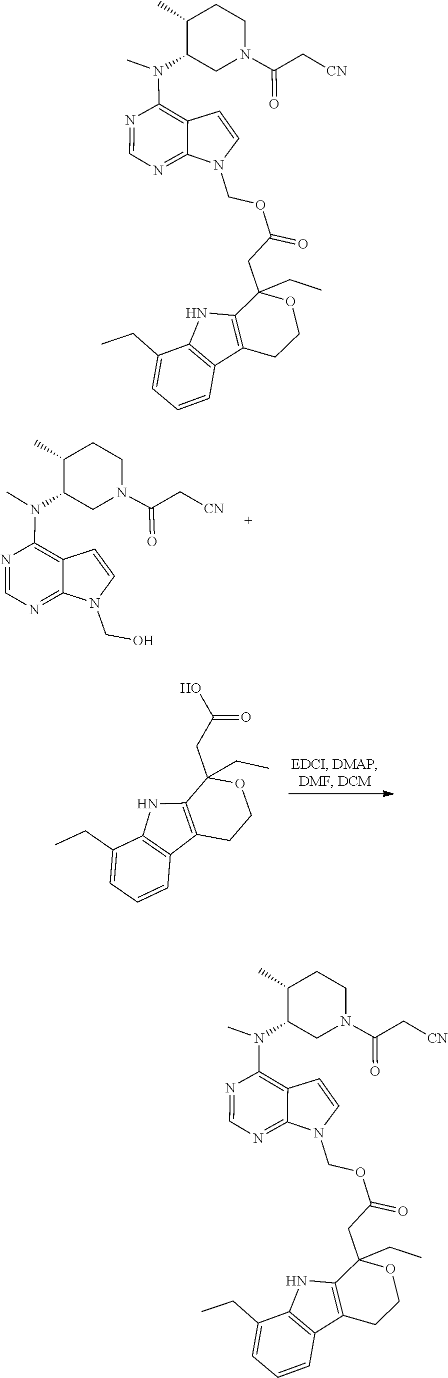 [SMILES: CCc1cccc2c3c([nH]c12)C(CC)(CC(=O)OCn1ccc2c(N(C)[C@H]4CN(C(=O)CC#N)CC[C@H]4C)ncnc21)OCC3 ; C[C@@H]1CCN(C(=O)CC#N)C[C@@H]1N(C)c1ncnc2c1ccn2CO ; CCc1cccc2c3c([nH]c12)C(CC)(CC(=O)O)OCC3 ; CCc1cccc2c3c([nH]c12)C(CC)(CC(=O)OCn1ccc2c(N(C)[C@H]4CN(C(=O)CC#N)CC[C@H]4C)ncnc21)OCC3]

### Synthesis of (4-(((3R, 4R)-1-(2-cyanoacetyl)-4-methylpiperidin-3-yl) (methyl) amino)-7H-pyrrolo [2, 3-d] pyrimidin-7-yl) methyl 2-(1, 8-diethyl-1, 3, 4, 9-tetrahydropyrano [3, 4-b] indol-1-yl) acetate

3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile (171 mg, 0.5 mmol), 4-dimethylamino pyridine (DMAP, 61 mg, 0.5 mmol), 1, 8-diethyl-1, 3, 4, 9-tetrahydropyran [3, 4-b] indole-1-acetic acid (etodolac, 216 mg, 0.75 mmol) and 1-(3-dimethylaminopropyl)-3-ethylcarbodiimide hydrochloride (EDCI, 144 mg, 0.75 mmol) were dissolved in a mixed solvent of dichloromethane (8.5 mL) and dimethylformamide (0.3 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.18 g, 58.9% yield. MS (m/z): [M+H]⁺ calcd for C₃₄H₄₁N₇O₄, 612.32; found, 612.3. ¹H NMR (400 MHz, Chloroform-d) δ 8.97 (d, J=13.6 Hz, 1H), 8.31 (d, J=8.1 Hz, 1H), 7.33 (d, J=7.6 Hz, 1H), 7.10-7.02 (m, 2H), 7.00 (d, J=7.3 Hz, 1H), 6.51 (d, J=4.1 Hz, 1H), 6.17 (dtd, J=15.9, 10.7, 4.1 Hz, 2H), 5.10 (dp, J=14.0, 4.6 Hz, 1H), 4.09-3.70 (m, 5H), 3.60-3.47 (m, 3H), 3.35 (d, J=14.9 Hz, 3H), 3.02 (d, J=16.2 Hz, 1H), 2.87 (ddd, J=16.8, 12.9, 5.8 Hz, 3H), 2.68 (dt, J=15.4, 4.3 Hz, 1H), 2.56-2.41 (m, 1H), 2.07 (dt, J=14.3, 6.8 Hz, 1H), 1.98 (s, 2H), 1.90 (d, J=24.6 Hz, 3H), 1.79-1.65 (m, 1H), 1.37 (ddd, J=10.0, 6.5, 2.4 Hz, 3H), 1.06 (dt, J=13.8, 6.3 Hz, 3H), 0.74 (t, J=7.3 Hz, 3H).

### Example 64

### (4-(((3R, 4R)-1-(2-cyanoacetyl)-4-methylpiperidin-3-yl) (methyl) amino)-7H-pyrrolo [2, 3-d] pyrimidin-7-yl) methyl (S)-2-((tert-butoxycarbonyl) amino)-2-phenylacetate

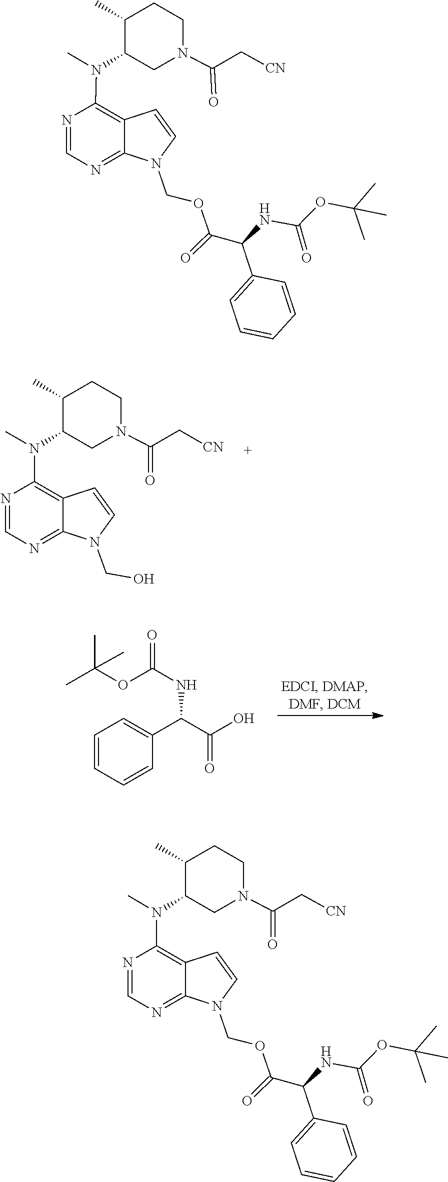

### Synthesis of (4-(((3R, 4R)-1-(2-cyanoacetyl)-4-methylpiperidin-3-yl) (methyl) amino)-7H-pyrrolo [2, 3-d] pyrimidin-7-yl) methyl (S)-2-((tert-butoxycarbonyl) amino)-2-phenylacetate

3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile (171 mg, 0.5 mmol), 4-dimethylaminopyridine (DMAP, 61 mg, 0.5 mmol), N-Boc-L-phenylglycine (189 mg, 0.75 mmol) and 1-(3-dimethylaminopropyl)-3-ethylcarbodiimide hydrochloride (EDCI, 144 mg, 0.75 mmol) was dissolved in a mixed solvent of dichloromethane (8.5 mL) and N, N-dimethylformamide (0.3 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.148 g, 51.4% yield. MS (m/z): [M+H]⁺ calcd for C₃₀H₃₇N₇O₅, 576.29; found, 576.3. ¹H NMR (400 MHz, Chloroform-d) δ 8.28 (d, J=9.3 Hz, 1H), 7.25 (d, J=6.9 Hz, 5H), 7.07 (dd, J=16.0, 3.9 Hz, 1H), 6.51 (dd, J=7.1, 3.7 Hz, 1H), 6.23-6.14 (m, 2H), 5.49 (d, J=7.5 Hz, 1H), 5.29 (d, J=7.5 Hz, 1H), 5.11 (dt, J=9.7, 4.5 Hz, 1H), 4.13-3.76 (m, 2H), 3.63-3.46 (m, 4H), 3.36 (d, J=17.3 Hz, 3H), 2.49 (tt, J=13.9, 6.0 Hz, 1H), 1.87 (dd, J=9.5, 4.6 Hz, 1H), 1.82-1.67 (m, 1H), 1.39 (s, 9H), 1.08 (dd, J=13.5, 7.1 Hz, 3H).

### Example 65

### (4-(((3R, 4R)-1-(2-cyanoacetyl)-4-methylpiperidin-3-yl) (methyl) amino)-7H-pyrrolo [2, 3-d] pyrimidin-7-yl) methyl 2-(4-(1-oxoisoindol-2-yl)) phenyl) butanoate

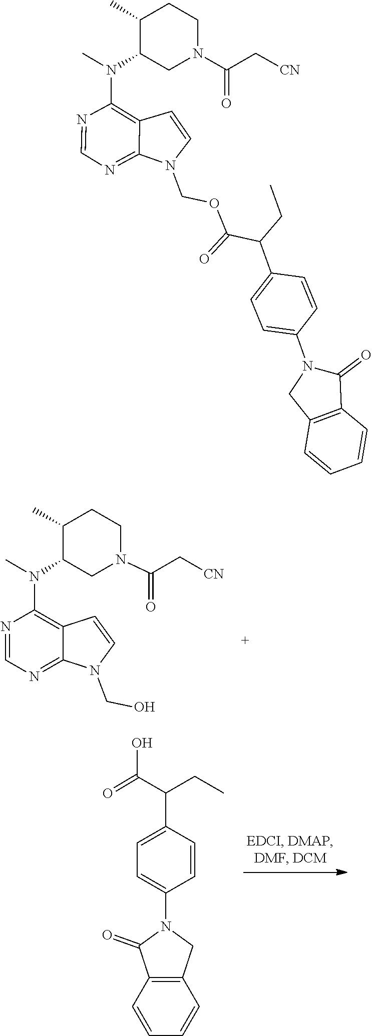

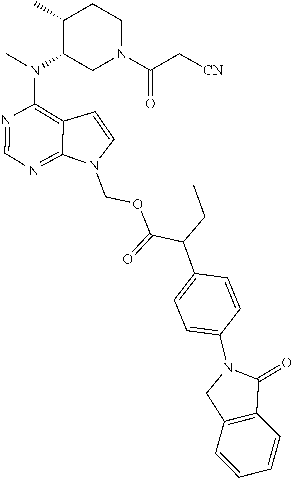 [SMILES: CCC(C(=O)OCn1ccc2c(N(C)[C@H]3CN(C(=O)CC#N)CC[C@H]3C)ncnc21)c1ccc(N2Cc3ccccc3C2=O)cc1]

### Synthesis of (4-(((3R, 4R)-1-(2-cyanoacetyl)-4-methylpiperidin-3-yl) (methyl) amino)-7H-pyrrolo [2, 3-d] pyrimidin-7-yl) methyl 2-(4-(1-oxoisoindol-2-yl)) phenyl) butanoate

3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile (20 mg, 0.058 mmol), 4-dimethylamino pyridine (DMAP, 4 mg, 0.03 mmol), 2-(4-(1-oxoisoindolin-2-yl) phenyl) butanoic acid (indobufen, 26 mg, 0.09 mmol) and 1-(3-dimethylaminopropyl)-3-ethylcarbodiimide hydrochloride (EDCI, 17 mg, 0.09 mmol) were dissolved in a mixed solvent of dichloromethane (0.5 mL) and N, N-dimethylformamide (0.05 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.012 g, 33.4% yield. MS (m/z): [M+H]⁺ calcd for C₃₅H₃₇N₇O₄, 620.29; found, 620.3. ¹H NMR (400 MHz, Chloroform-d) δ 8.30 (d, J=8.7 Hz, 1H), 7.90 (d, J=7.5 Hz, 1H), 7.80-7.69 (m, 2H), 7.59 (t, J 7.5 Hz, 1H), 7.51 (d, J 8.4 Hz, 2H), 7.33-7.22 (m, 2H), 7.14-7.06 (m, 1H), 6.53-6.45 (m, 1H), 6.15 (dt, J=18.9, 10.9 Hz, 2H), 5.08 (s, 1H), 4.82 (s, 2H), 4.05 (d, J 13.5 Hz, 1H), 3.92-3.66 (i, 2H), 3.53 (q, J=6.6, 5.1 Hz, 3H), 3.37-3.27 (i, 3H), 2.54-2.40 (i, 1H), 2.07 (dq, J=14.1, 7.3 Hz, 2H), 1.93 (s, 1H), 1.76 (tt, J=19.7, 8.8 Hz, 2H), 1.06 (s, 3H), 0.82 (s, 3H).

### Example 66

### (4-(((3R, 4R)-1-(2-cyanoacetyl)-4-methylpiperidin-3-yl) (methyl) amino)-7H-pyrrolo [2, 3-d] pyrimidin-7-yl) methyl 2-((3-chloro-2-methylphenyl) amino) benzoate

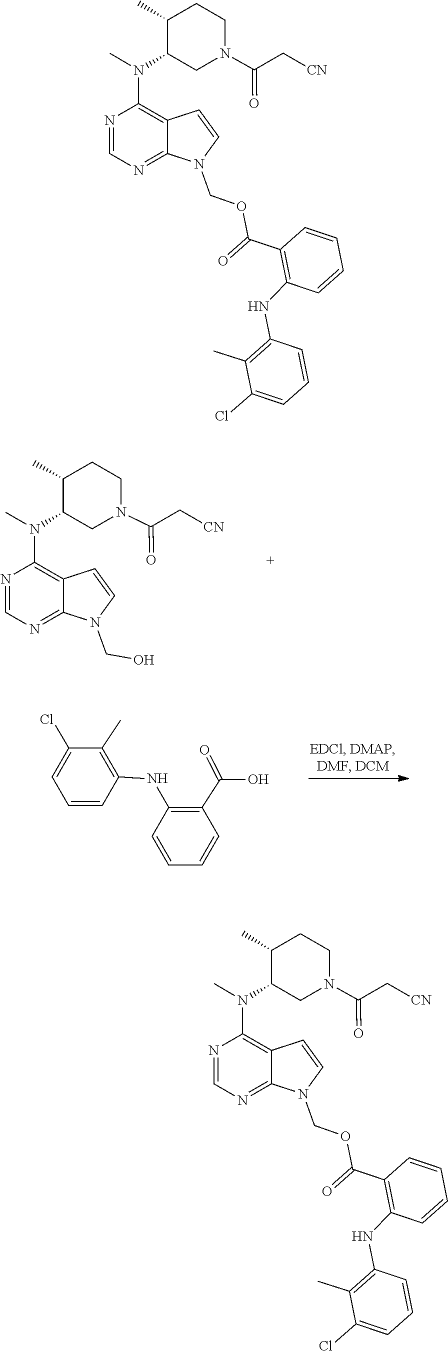 [SMILES: Cc1c(Cl)cccc1Nc1ccccc1C(=O)OCn1ccc2c(N(C)[C@H]3CN(C(=O)CC#N)CC[C@H]3C)ncnc21 ; C[C@@H]1CCN(C(=O)CC#N)C[C@@H]1N(C)c1ncnc2c1ccn2CO ; Cc1c(Cl)cccc1Nc1ccccc1C(=O)O ; Cc1c(Cl)cccc1Nc1ccccc1C(=O)OCn1ccc2c(N(C)[C@H]3CN(C(=O)CC#N)CC[C@H]3C)ncnc21]

### Synthesis of (4-(((3R, 4R)-1-(2-cyanoacetyl)-4-methylpiperidin-3-yl) (methyl) amino)-7H-pyrrolo [2, 3-d] pyrimidin-7-yl) methyl 2-((3-chloro-2-methylphenyl) amino) benzoate

3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile (200 mg, 0.58 mmol), 4-dimethylamino pyridine (DMAP, 35 mg, 0.29 mmol), 2-((3-chloro-2-methylphenyl) amino) benzoic acid (tolfenamic acid, 230 mg, 0.88 mmol) and 1-(3-dimethylaminopropyl)-3-ethylcarbodiimide hydrochloride (EDCI, 168 mg, 0.88 mmol) were dissolved in a mixed solvent of dichloromethane (10 mL) and N, N-dimethylformamide (0.2 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further purified by silica gel column chromatography (petroleum ether/ethyl acetate=2:1 to 1:1) to give the title compound as a yellow solid, 0.145 g, 42.7% yield. MS (m/z): [M+H]⁺ calcd for C₃₁H₃₂ClN₇O₃, 586.23; found, 586.3. ¹H NMR (400 MHz, Chloroform-d) δ 9.20 (s, 1H), 8.37 (d, J=7.1 Hz, 1H), 7.95 (dd, J=8.2, 1.7 Hz, 1H), 7.30-7.17 (m, 4H), 7.12 (t, J=7.9 Hz, 1H), 6.75 (d, J=8.5 Hz, 1H), 6.65 (t, J=7.6 Hz, 1H), 6.60-6.55 (m, 1H), 6.42 (d, J=3.3 Hz, 2H), 5.13 (q, J=6.0, 5.5 Hz, 1H), 4.12-3.75 (m, 2H), 3.64-3.56 (m, 1H), 3.53-3.46 (m, 2H), 3.38 (d, J=16.8 Hz, 3H), 2.58-2.44 (m, 1H), 2.32 (s, 3H), 1.97-1.82 (m, 2H), 1.81-1.68 (m, 1H), 1.08 (dd, J=12.9, 7.0 Hz, 3H).

### Example 67

### (4-(((3R, 4R)-1-(2-cyanoacetyl)-4-methylpiperidin-3-yl) (methyl) amino)-7H-pyrrolo [2, 3-d] pyrimidin-7-yl) methyl 2-((2, 3-dimethylphenyl) amino) benzoate

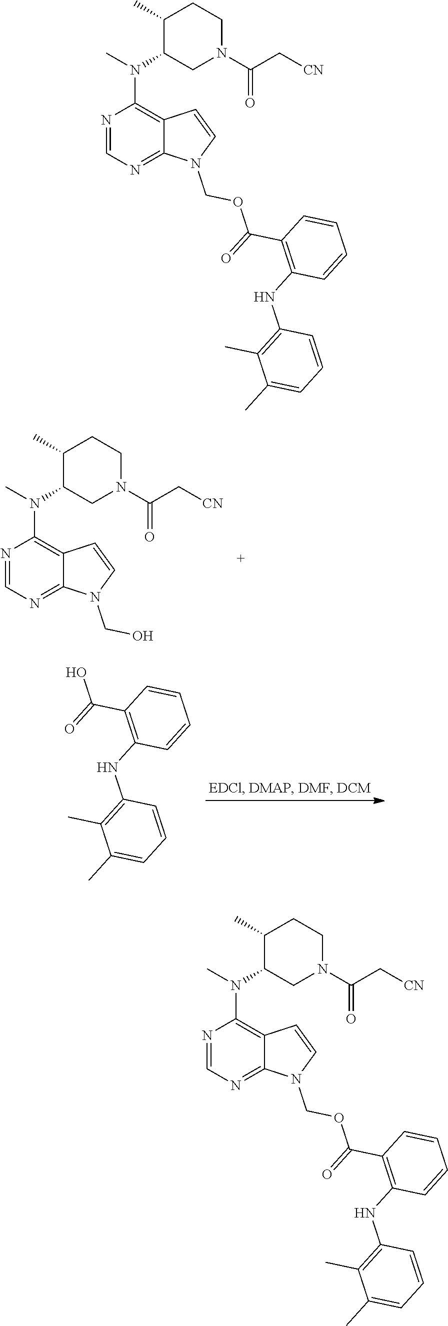 [SMILES: Cc1cccc(Nc2ccccc2C(=O)OCn2ccc3c(N(C)[C@H]4CN(C(=O)CC#N)CC[C@H]4C)ncnc32)c1C ; C[C@@H]1CCN(C(=O)CC#N)C[C@@H]1N(C)c1ncnc2c1ccn2CO ; Cc1cccc(Nc2ccccc2C(=O)O)c1C ; Cc1cccc(Nc2ccccc2C(=O)OCn2ccc3c(N(C)[C@H]4CN(C(=O)CC#N)CC[C@H]4C)ncnc32)c1C]

### Synthesis of (4-(((3R, 4R)-1-(2-cyanoacetyl)-4-methylpiperidin-3-yl) (methyl) amino)-7H-pyrrolo [2, 3-d] pyrimidin-7-yl) methyl 2-((2, 3-dimethylphenyl) amino) benzoate

3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile (171 mg, 0.5 mmol), 4-dimethylaminopyridine (DMAP, 61 mg, 0.5 mmol), 2-((2, 3-dimethylphenyl) amino) benzoic acid (mefenamic acid, 181 mg, 0.75 mmol) and 1-(3-dimethylaminopropyl)-3-ethylcarbodiimide hydrochloride (EDCI, 144 mg, 0.75 mmol) were dissolved in a mixed solvent of dichloromethane (8.5 mL) and N, N-dimethylformamide (0.3 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a yellow solid, 0.012 g, 45.6% yield. MS (m/z): [M+H]⁺ calcd for C₃₂H₃₅N₇O₃, 566.28; found, 566.3. ¹H NMR (400 MHz, Chloroform-d) δ 9.14 (s, 1H), 8.37 (d, J=7.1 Hz, 1H), 7.93 (dd, J=8.1, 1.7 Hz, 1H), 7.29 (dd, J=15.0, 3.7 Hz, 1H), 7.25-7.16 (m, 1H), 7.16-7.06 (m, 2H), 7.03 (d, J=6.8 Hz, 1H), 6.68 (d, J=8.6 Hz, 1H), 6.63-6.53 (m, 2H), 6.41 (d, J=3.0 Hz, 2H), 5.12 (dt, J=9.7, 4.7 Hz, 1H), 4.05 (dd, J=13.2, 4.4 Hz, 1H), 3.78 (ddd, J=15.8, 13.2, 8.1 Hz, 1H), 3.58 (td, J=11.3, 10.3, 5.0 Hz, 1H), 3.54-3.47 (m, 2H), 3.37 (d, J=15.3 Hz, 3H), 2.57-2.41 (m, 1H), 2.33 (s, 3H), 2.17 (s, 3H), 1.99 (s, 1H), 1.97-1.84 (m, 1H), 1.72 (dtd, J=32.5, 6.4, 3.6 Hz, 1H), 1.07 (dd, J=13.3, 7.0 Hz, 3H).

### Example 68

### N-methyl-1-((trans)-4-(methyl (7-(2-(3-phenoxyphenyl) propanoyl)-7H-pyrrolo [2, 3-d] pyrimidin-4-yl) amino) cyclohexyl) methanesulfonamide

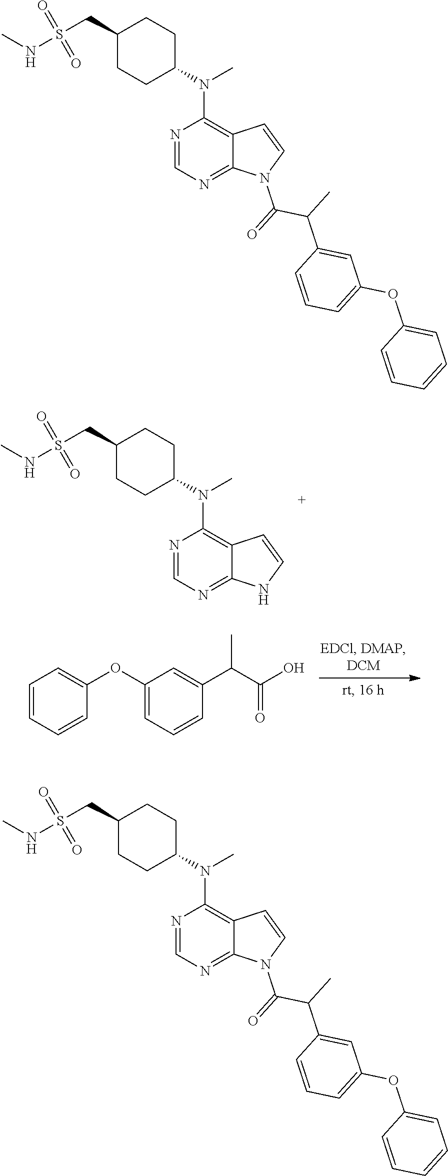 [SMILES: CNS(=O)(=O)C[C@H]1CC[C@H](N(C)c2ncnc3c2ccn3C(=O)C(C)c2cccc(Oc3ccccc3)c2)CC1 ; CNS(=O)(=O)C[C@H]1CC[C@H](N(C)c2ncnc3[nH]ccc23)CC1 ; CC(C(=O)O)c1cccc(Oc2ccccc2)c1 ; CNS(=O)(=O)C[C@H]1CC[C@H](N(C)c2ncnc3c2ccn3C(=O)C(C)c2cccc(Oc3ccccc3)c2)CC1]

### Synthesis of N-methyl-1-((trans)-4-(methyl (7-(2-(3-phenoxyphenyl) propanoyl)-7H-pyrrolo [2, 3-d] pyrimidin-4-yl) amino) cyclohexyl) methanesulfonamide

N-methyl-1-((trans)-4-(methyl (7H-pyrrolo [2, 3-d] pyrimidin-4-yl) amino) cyclohexyl) methane sulfonamide (oclacitinib, 168 mg, 0.5 mmol), 4-dimethylamino pyridine (DMAP, 61 mg, 0.5 mmol), 2-(3-phenoxyphenyl) propanoic acid (fenoprofen, 158 mg, 0.65 mmol) and 1-(3-dimethylaminopropyl)-3-ethylcarbodiimide hydrochloride (EDCI, 144 mg, 0.75 mmol) were dissolved in dichloromethane (15 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 5:3) to give the title compound as a white solid, 0.18 g, 64.1% yield. MS (m/z): [M+H]⁺ calcd for C₃₀H₃₅N₅O₄S, 562.24; found, 562.4. ¹H NMR (400 MHz, DMSO-d₆) δ 8.26 (s, 1H), 7.65 (d, J=4.2 Hz, 1H), 7.40-7.33 (m, 2H), 7.29 (t, J=7.9 Hz, 1H), 7.18-7.11 (m, 2H), 7.05 (t, J=2.1 Hz, 1H), 6.98-6.90 (m, 2H), 6.90-6.78 (m, 3H), 6.06 (q, J=6.9 Hz, 1H), 4.64 (s, 1H), 3.15 (s, 3H), 2.95 (d, J=6.2 Hz, 2H), 2.60 (d, J=5.0 Hz, 3H), 2.10-2.02 (m, 2H), 1.92-1.77 (m, 1H), 1.70 (tt, J=8.2, 3.1 Hz, 4H), 1.53 (d, J=7.0 Hz, 3H), 1.29 (d, J=12.9 Hz, 2H).

### Example 69

### Methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-(2-fluoro-[1, 1′-biphenyl]-4-yl) propanoate

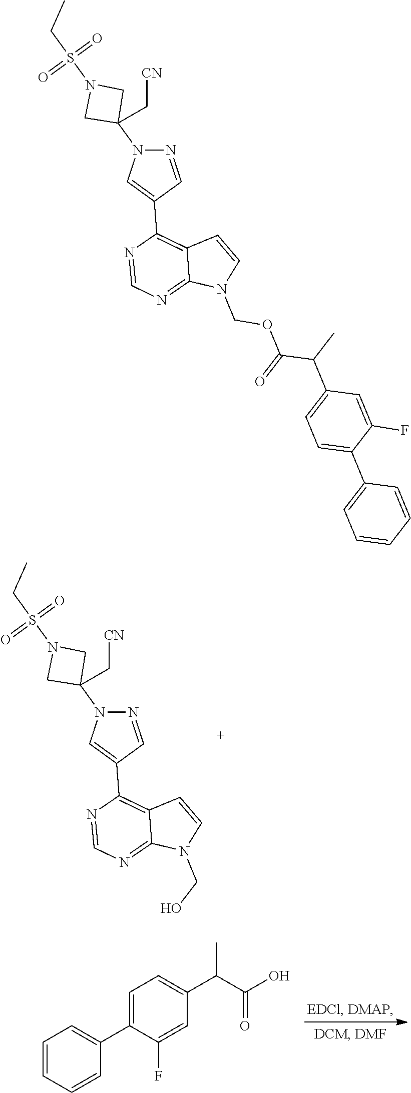 [SMILES: CCS(=O)(=O)N1CC(CC#N)(n2cc(-c3ncnc4c3ccn4COC(=O)C(C)c3ccc(-c4ccccc4)c(F)c3)cn2)C1 ; CCS(=O)(=O)N1CC(CC#N)(n2cc(-c3ncnc4c3ccn4CO)cn2)C1 ; CC(C(=O)O)c1ccc(-c2ccccc2)c(F)c1]

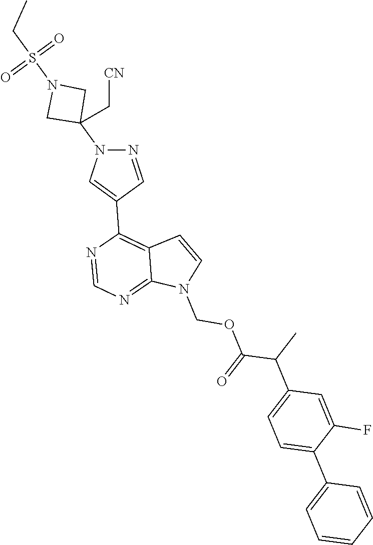 [SMILES: CCS(=O)(=O)N1CC(CC#N)(n2cc(-c3ncnc4c3ccn4COC(=O)C(C)c3ccc(-c4ccccc4)c(F)c3)cn2)C1]

### Synthesis of methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-(2-fluoro-[1, 1′-biphenyl]-4-yl) propanoate

2-(1-(Ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile (201 mg, 0.5 mmol), 4-dimethylamino pyridine (DMAP, 31 mg, 0.25 mmol), 2-(2-fluoro-4-biphenyl) propanoic acid (flurbiprofen, 146 mg, 0.6 mmol) and 1-(3-dimethylaminopropyl)-3-ethylcarbodiimide hydrochloride (EDCI, 144 mg, 0.75 mmol) were dissolved in dichloromethane (10 mL) and N, N-dimethylformamide (1 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:2) to give the title compound as a white solid, 0.27 g, 86.1% yield. MS (m/z): [M+H]⁺ calcd for C₃₂H₃₀FN₇O₄S, 628.21; found, 628.3. ¹H NMR (400 MHz, DMSO-d₆) δ 8.95 (s, 1H), 8.78 (s, 1H), 8.48 (s, 1H), 7.75 (d, J=3.8 Hz, 1H), 7.50-7.41 (m, 4H), 7.40-7.32 (m, 2H), 7.19 (d, J=3.8 Hz, 1H), 7.17-7.08 (m, 2H), 6.37-6.24 (m, 2H), 4.60 (d, J=9.1 Hz, 2H), 4.25 (d, J=9.1 Hz, 2H), 3.93 (q, J=7.0 Hz, 1H), 3.69 (s, 2H), 3.23 (q, J=7.3 Hz, 2H), 1.40 (d, J=7.1 Hz, 3H), 1.25 (t, J=7.2 Hz, 3H).

### Example 70

### Methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-(2-((2, 6-dichlorophenyl) amino) phenyl) acetate

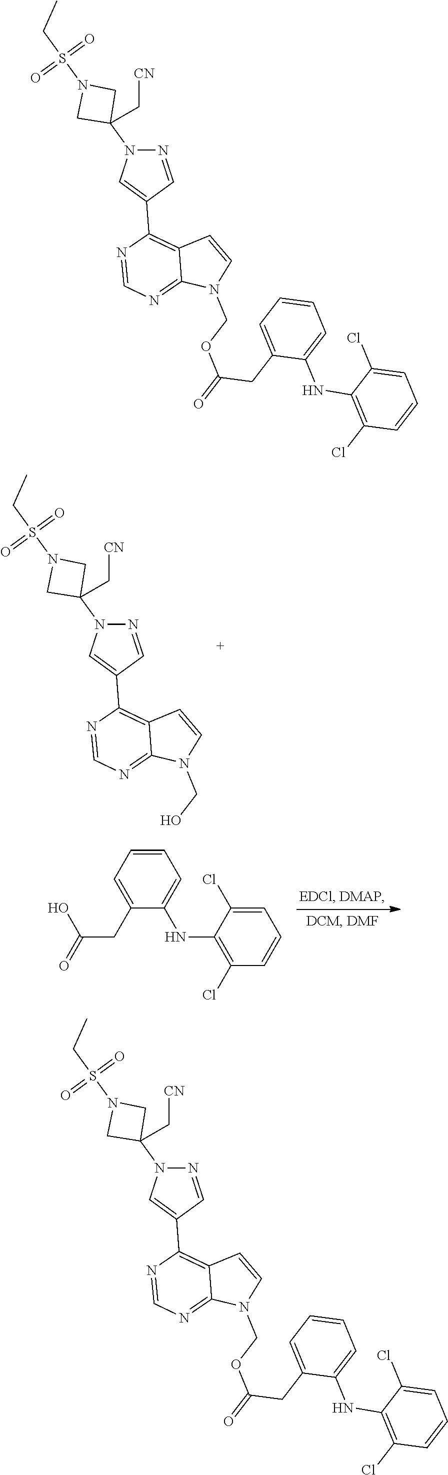

### Synthesis of methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-(2-((2, 6-dichlorophenyl) amino) phenyl) acetate

2-(1-(Ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile (201 mg, 0.5 mmol), 4-dimethylamino pyridine (DMAP, 31 mg, 0.25 mmol), 2-(2, 6-dichloroanilino) phenylacetic acid (diclofenac, 148 mg, 0.5 mmol) and 1-(3-dimethylaminopropyl)-3-ethylcarbodiimide hydrochloride (EDCI, 144 mg, 0.75 mmol) were dissolved in dichloromethane (10 mL) and N, N-dimethylformamide (2 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=10:1 to 1:2) to give the title compound as a white solid, 0.07 g, 20.6% yield. MS (m/z): [M+H]⁺ calcd for C₃₁H₂₈Cl₂N₈O₄S, 679.13; found, 679.1.1H NMR (400 MHz, DMSO-d₆) δ 8.96 (s, 1H), 8.80 (s, 1H), 8.50 (s, 1H), 7.76 (d, J=3.8 Hz, 1H), 7.49 (d, J=8.1 Hz, 2H), 7.24-7.12 (m, 3H), 7.04 (td, J=7.7, 1.6 Hz, 1H), 6.92 (s, 1H), 6.81 (td, J=7.4, 1.2 Hz, 1H), 6.32 (s, 2H), 6.22 (d, J=8.0 Hz, 1H), 4.61 (d, J=9.1 Hz, 2H), 4.26 (d, J=9.1 Hz, 2H), 3.86 (s, 2H), 3.70 (s, 2H), 3.24 (q, J=7.4 Hz, 2H), 1.26 (t, J=7.4 Hz, 3H).

### Example 71

### Methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-(3-phenoxyphenyl) propanoate

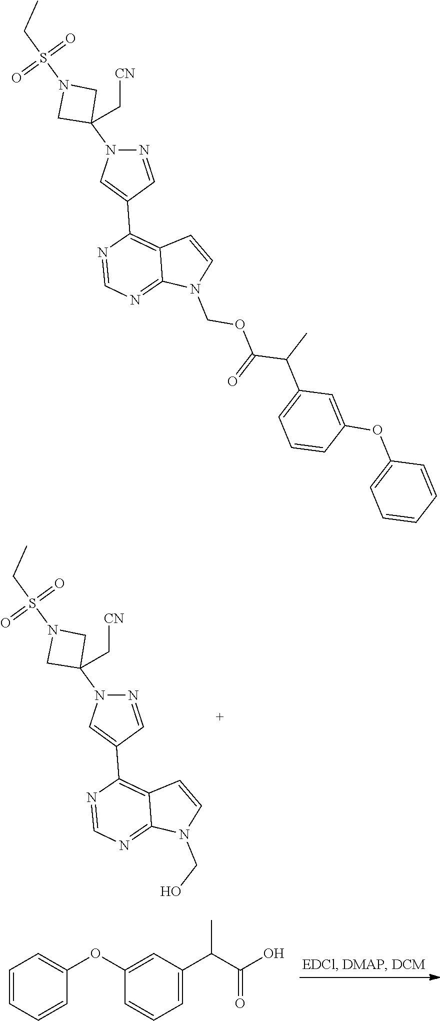

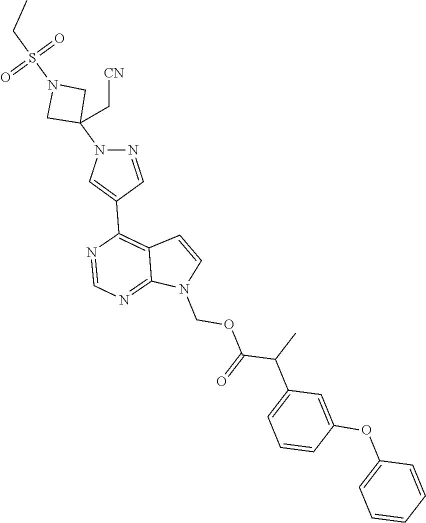 [SMILES: CCS(=O)(=O)N1CC(CC#N)(n2cc(-c3ncnc4c3ccn4COC(=O)C(C)c3cccc(Oc4ccccc4)c3)cn2)C1]

### Synthesis of methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-(3-phenoxyphenyl) propanoate

2-(1-(Ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile (201 mg, 0.5 mmol), 4-dimethylamino pyridine (DMAP, 31 mg, 0.25 mmol), 2-(3-phenoxyphenyl) propanoic acid (fenoprofen, 1345 mg, 0.6 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:2) to give the title compound as a white solid, 0.22 g, 70.4% yield. MS (m/z): [M+H]⁺ calcd for C₃₂H₃₁N₇O₅S, 626.21; found, 626.1. ¹H NMR (400 MHz, DMSO-d₆) δ 8.96 (s, 1H), 8.78 (s, 1H), 8.50 (s, 1H), 7.71 (d, J=3.8 Hz, 1H), 7.39-7.30 (m, 2H), 7.24 (t, J=7.9 Hz, 1H), 7.19 (d, J=3.8 Hz, 1H), 7.11 (t, J=7.4 Hz, 1H), 6.97 (d, J=7.7 Hz, 1H), 6.90 (d, J=8.0 Hz, 2H), 6.85 (t, J=2.1 Hz, 1H), 6.79 (dd, J=8.1, 2.5 Hz, 1H), 6.27 (d, J=4.3 Hz, 2H), 4.61 (d, J=9.1 Hz, 2H), 4.26 (d, J=9.1 Hz, 2H), 3.84 (q, J=7.1 Hz, 1H), 3.70 (s, 2H), 3.24 (q, J=7.3 Hz, 2H), 1.35 (d, J=7.1 Hz, 3H), 1.25 (t, J=7.2 Hz, 3H).

### Example 72

### Methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-(4-((2-oxocyclopentyl) methyl) phenyl) propanoate

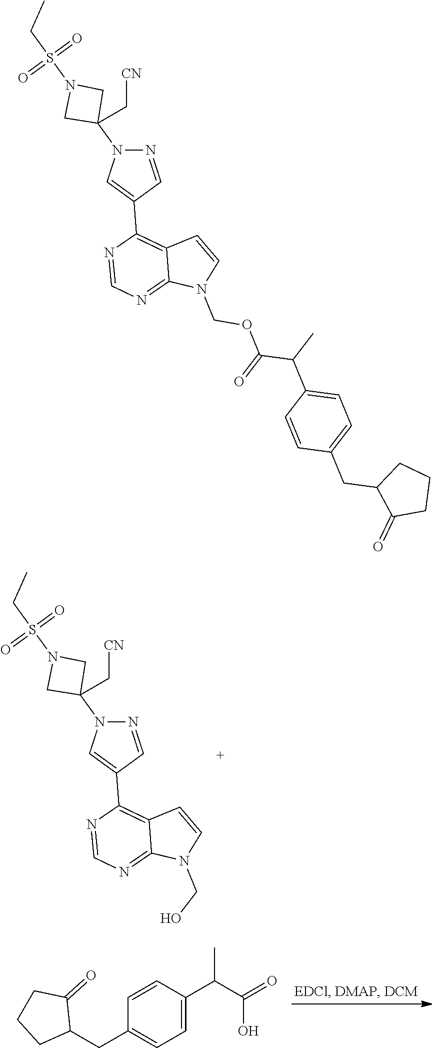 [SMILES: CCS(=O)(=O)N1CC(CC#N)(n2cc(-c3ncnc4c3ccn4COC(=O)C(C)c3ccc(CC4CCCC4=O)cc3)cn2)C1 ; CCS(=O)(=O)N1CC(CC#N)(n2cc(-c3ncnc4c3ccn4CO)cn2)C1 ; CC(C(=O)O)c1ccc(CC2CCCC2=O)cc1]

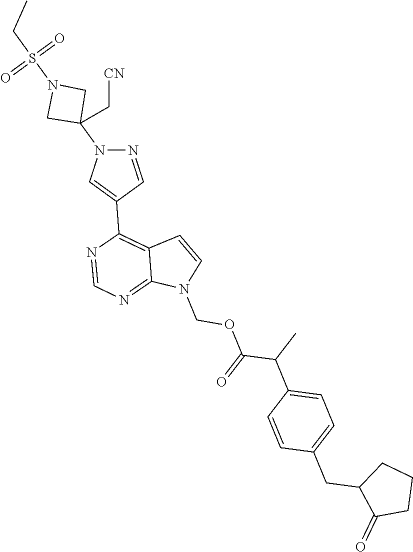 [SMILES: CCS(=O)(=O)N1CC(CC#N)(n2cc(-c3ncnc4c3ccn4COC(=O)C(C)c3ccc(CC4CCCC4=O)cc3)cn2)C1]

### Synthesis of methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-(4-((2-oxocyclopentyl) methyl) phenyl) propanoate

2-(1-(Ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile (201 mg, 0.5 mmol), 4-dimethylamino pyridine (DMAP, 31 mg, 0.25 mmol), 2-[4-(2-oxocyclopentan-1-ylmethyl) phenyl] propanoic acid (loxoprofen, 148 mg, 06 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:2) to give the title compound as a white solid, 0.26 g, 82.6% yield. MS (m/z): [M+H]⁺ calcd for C₃₂H₃₅N₇O₅S, 630.24; found, 630.1. ¹H NMR (400 MHz, DMSO-d₆) δ 8.96 (s, 1H), 8.78 (d, J=1.6 Hz, 1H), 8.49 (s, 1H), 7.71 (d, J=3.8 Hz, 1H), 7.17 (d, J=3.8 Hz, 1H), 7.04 (ddd, J=25.3, 8.1, 2.3 Hz, 4H), 6.26 (d, J=4.2 Hz, 2H), 4.60 (d, J=9.1 Hz, 2H), 4.25 (d, J=9.1 Hz, 2H), 3.77 (q, J=7.1 Hz, 1H), 3.69 (s, 2H), 3.24 (q, J=7.3 Hz, 2H), 2.87 (dd, J=13.3, 3.7 Hz, 1H), 2.40-2.15 (m, 3H), 2.04 (dd, J=10.2, 8.5 Hz, 1H), 1.80 (d, J=12.9 Hz, 2H), 1.70-156 (m, 1H), 1.44-1.31 (m, 4H), 1.28-1.23 (m, 3H).

### Example 73

### Methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-(1-(4-chlorobenzoyl)-5-methoxy-2-methyl-1H-indol-3-yl) acetate

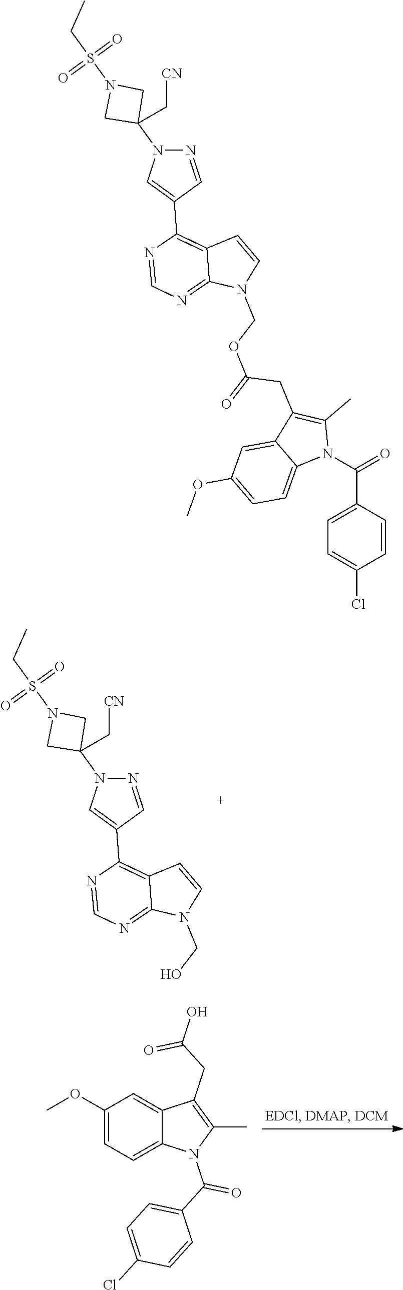

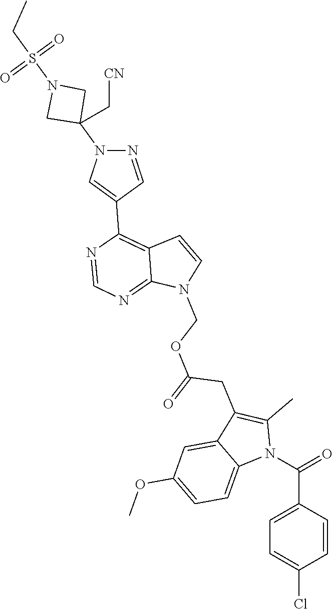 [SMILES: CCS(=O)(=O)N1CC(CC#N)(n2cc(-c3ncnc4c3ccn4COC(=O)Cc3c(C)n(C(=O)c4ccc(Cl)cc4)c4ccc(OC)cc34)cn2)C1]

### Synthesis of methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-(1-(4-chlorobenzoyl)-5-methoxy-2-methyl-1H-indol-3-yl) acetate

2-(1-(Ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile (201 mg, 0.5 mmol), 4-dimethylamino pyridine (DMAP, 31 mg, 0.25 mmol), 1-(4-chlorobenzoyl)-5-methoxy-2-methyl-1H-indole-3-acetic acid (indomethacin, 214 mg, 0.6 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:2) to give the title compound as a yellow solid, 0.26 g, 75.6% yield. MS (m/z): [M+H]⁺ calcd for C₃₆H₃₃ClN₈O₆S, 741.19; found, 741.1. ¹H NMR (400 MHz, DMSO-d₆) δ 8.98 (s, 1H), 8.79 (s, 1H), 8.52 (s, 1H), 7.76 (d, J=3.8 Hz, 1H), 7.63 (s, 4H), 7.21 (d, J=3.8 Hz, 1H), 6.96-6.87 (m, 2H), 6.68 (dd, J=9.0, 2.5 Hz, 1H), 6.31 (s, 2H), 4.63 (d, J=9.2 Hz, 2H), 4.27 (d, J=9.1 Hz, 2H), 3.83 (s, 2H), 3.72 (s, 2H), 3.67 (s, 3H), 3.25 (q, J=7.4 Hz, 2H), 2.14 (s, 3H), 1.27 (t, J=7.3 Hz, 3H).

### Example 74

### Methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) (S)-2-((tert-butoxycarbonyl) amino)-2-phenylacetate

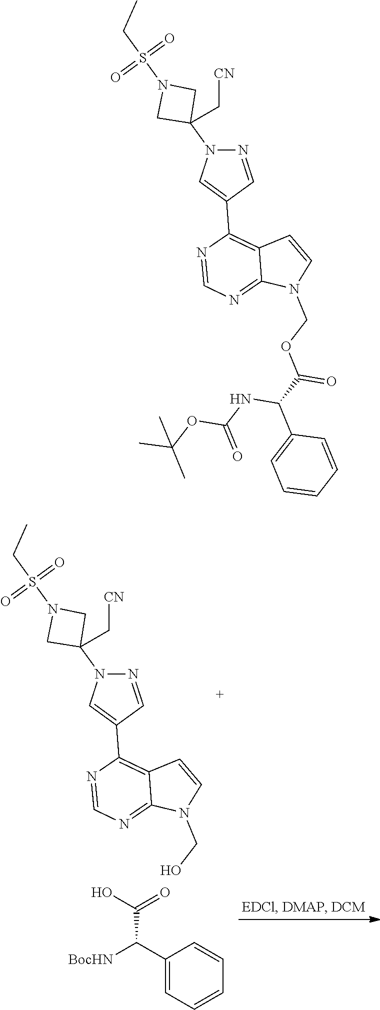

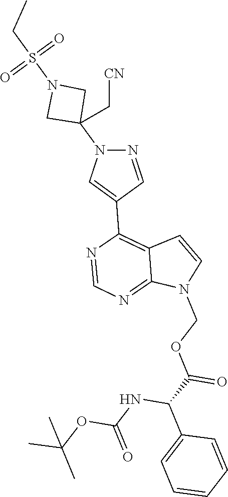 [SMILES: CCS(=O)(=O)N1CC(CC#N)(n2cc(-c3ncnc4c3ccn4COC(=O)[C@@H](NC(=O)OC(C)(C)C)c3ccccc3)cn2)C1]

Synthesis of methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) (S)-2-((tert-butoxycarbonyl) amino)-2-phenylacetate

2-(1-(Ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile (201 mg, 0.5 mmol), 4-dimethylamino pyridine (DMAP, 31 mg, 0.25 mmol), N-Boc-L-phenylglycine (151 mg, 0.6 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:2) to give the title compound as a white solid, 0.23 g, 72.5% yield. MS (m/z): [M+H]⁺ calcd for C₃₀H₃₄N₈O₆S, 635.23; found, 635.2. ¹H NMR (400 MHz, DMSO-d₆) δ 8.97 (s, 1H), 8.79 (s, 1H), 8.51 (s, 1H), 7.79 (d, J=7.8 Hz, 1H), 7.70 (s, 1H), 7.41-7.16 (m, 6H), 6.42-6.24 (m, 2H), 5.20 (d, J=7.8 Hz, 1H), 4.63 (d, J=9.1 Hz, 2H), 4.27 (d, J=9.2 Hz, 2H), 3.71 (s, 2H), 3.25 (q, J=7.3 Hz, 2H), 1.38-1.21 (m, 12H).

### Example 75

### (R)-3-cyclopentyl-3-(4-(7-(2-(2-((2, 6-dichlorophenyl) amino) phenyl) acetyl)-7H-pyrrolo [2, 3-d] pyrimidin-4-yl)-1H-pyrazol-1-yl) propanenitrile

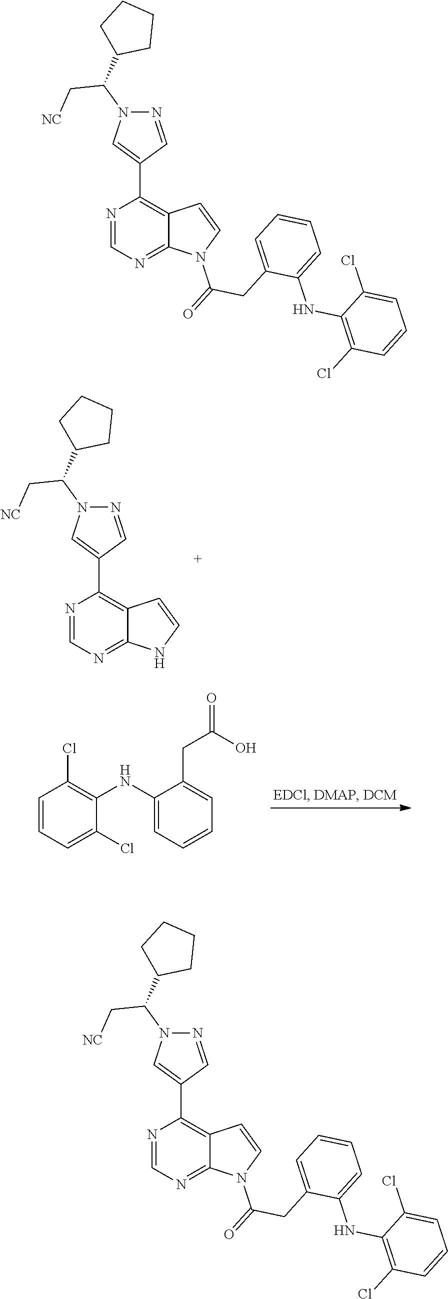 [SMILES: N#CC[C@H](C1CCCC1)n1cc(-c2ncnc3c2ccn3C(=O)Cc2ccccc2Nc2c(Cl)cccc2Cl)cn1 ; N#CC[C@H](C1CCCC1)n1cc(-c2ncnc3[nH]ccc23)cn1 ; O=C(O)Cc1ccccc1Nc1c(Cl)cccc1Cl ; N#CC[C@H](C1CCCC1)n1cc(-c2ncnc3c2ccn3C(=O)Cc2ccccc2Nc2c(Cl)cccc2Cl)cn1]

### Synthesis of (R)-3-cyclopentyl-3-(4-(7-(2-(2-((2, 6-dichlorophenyl) amino) phenyl) acetyl)-7H-pyrrolo [2, 3-d] pyrimidin-4-yl)-1H-pyrazol-1-yl) propanenitrile

(R)-3-(4-(7h-pyrrolo [2, 3-d] pyridin-4-yl)-1h-pyrazol-1-yl)-3-cyclopentylpropane (Ruxolitinib, 184 mg, 0.6 mmol), 4-dimethylamino pyridine (DMAP, 7 mg, 0.06 mmol), 2-(2, 6-dichloroanilino) phenylacetic acid (diclofenac, 231 mg, 0.78 mmol) and 1-(3-dimethylaminopropyl)-3-ethylcarbodiimide hydrochloride (EDCI, 173 mg, 0.9 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=10:1 to 5:1) to give the title compound as a white solid, 0.11 g, 31.4% yield. MS (m/z): [M+H]⁺ calcd for C₃₁H₂₇Cl₂N₇O, 584.17; found, 584.1. ¹H NMR (400 MHz, DMSO-d₆) δ 8.99 (s, 1H), 8.91 (s, 1H), 8.45 (s, 1H), 8.17 (d, J=4.1 Hz, 1H), 7.51 (d, J=8.1 Hz, 2H), 7.41-7.29 (m, 2H), 7.21 (dd, J=17.2, 9.1 Hz, 2H), 7.15-7.02 (m, 1H), 6.84 (t, J=7.4 Hz, 1H), 6.25 (d, J=8.1 Hz, 1H), 5.01 (s, 2H), 4.56 (td, J=9.7, 4.2 Hz, 1H), 3.31-3.17 (m, 2H), 2.45 (q, J=8.5 Hz, 1H), 1.83 (dtd, J=12.2, 7.5, 3.9 Hz, 1H), 1.68-1.41 (m, 4H), 1.39-1.21 (m, 3H).

### Example 76

### Methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-(4-(1-oxoisoindolin-2-yl) phenyl) butanoate

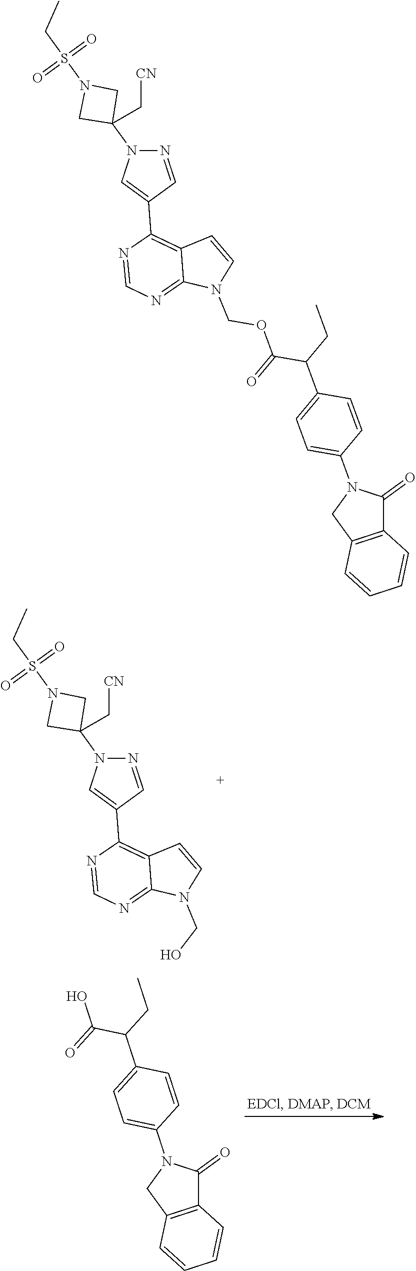

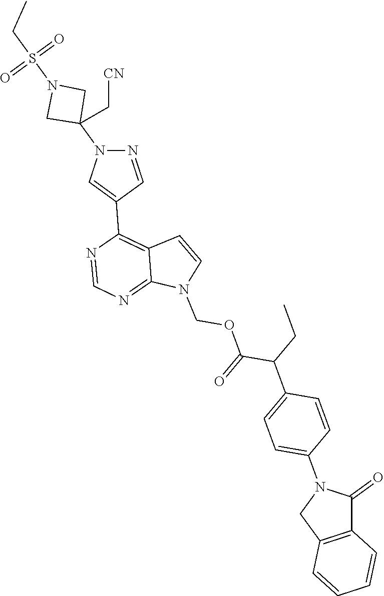 [SMILES: CCC(C(=O)OCn1ccc2c(-c3cnn(C4(CC#N)CN(S(=O)(=O)CC)C4)c3)ncnc21)c1ccc(N2Cc3ccccc3C2=O)cc1]

### Synthesis of methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-(4-(1-oxoisoindolin-2-yl) phenyl) butanoate

2-(1-(Ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile (201 mg, 0.5 mmol), 4-dimethylamino pyridine (DMAP, 31 mg, 0.25 mmol), 2-(4-(1-oxoisoindolin-2-yl) phenyl) butanoic acid (indobufen, 177 mg, 0.6 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to give the title compound as a white solid, 0.28 g, 82.5% yield. MS (m/z): [M+H]⁺ calcd for C₃₅H₃₄N₈O₅S, 679.24; found, 679.2. ¹H NMR (400 MHz, DMSO-d₆) δ 8.98 (s, 1H), 8.83 (s, 1H), 8.49 (s, 1H), 7.87-7.77 (m, 4H), 7.77-7.66 (m, 2H), 7.62-7.53 (m, 1H), 7.36-7.27 (m, 2H), 7.23 (d, J=3.8 Hz, 1H), 6.32 (q, J=10.8 Hz, 2H), 5.00 (s, 2H), 4.63 (d, J=9.1 Hz, 2H), 4.28 (d, J=9.1 Hz, 2H), 3.72 (s, 2H), 3.63 (t, J=7.6 Hz, 1H), 3.28 (q, J=7.3 Hz, 2H), 2.04-1.97 (m, 1H), 1.76 (dt, J=13.6, 7.0 Hz, 1H), 1.32-1.28 (m, 3H), 0.82 (t, J=7.3 Hz, 3H).

### Example 77

### (3R)-3-cyclopentyl-3-(4-(7-(2-(4-((2-oxocyclopentyl) methyl) phenyl) propanoyl)-7H-pyrrolo [2, 3-d] pyrimidin-4-yl)-1H-pyrazol-1-yl) propanenitrile

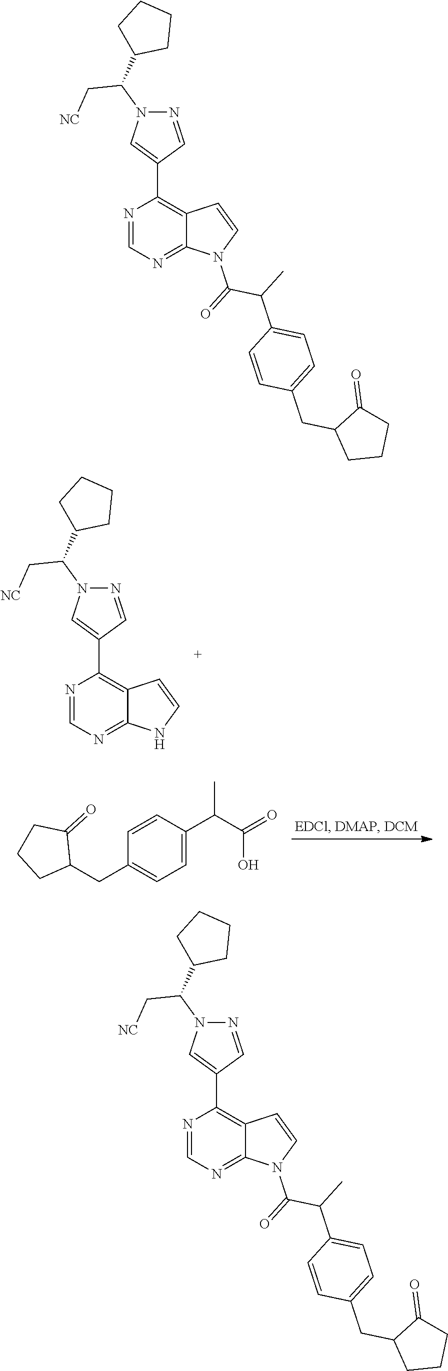 [SMILES: CC(C(=O)n1ccc2c(-c3cnn([C@H](CC#N)C4CCCC4)c3)ncnc21)c1ccc(CC2CCCC2=O)cc1 ; N#CC[C@H](C1CCCC1)n1cc(-c2ncnc3[nH]ccc23)cn1 ; CC(C(=O)O)c1ccc(CC2CCCC2=O)cc1 ; CC(C(=O)n1ccc2c(-c3cnn([C@H](CC#N)C4CCCC4)c3)ncnc21)c1ccc(CC2CCCC2=O)cc1]

### Synthesis of (3R)-3-cyclopentyl-3-(4-(7-(2-(4-((2-oxocyclopentyl) methyl) phenyl) propanoyl)-7H-pyrrolo [2, 3-d] pyrimidin-4-yl)-1H-pyrazol-1-yl) propanenitrile

(R)-3-(4-(7h-pyrrolo [2, 3-d] pyridin-4-yl)-1h-pyrazol-1-yl)-3-cyclopentylpropane (Ruxolitinib, 184 mg, 0.6 mmol), 4-dimethylamino pyridine (DMAP, 7 mg, 0.06 mmol), 2-[4-(2-oxocyclopentan-1-ylmethyl) phenyl] propanoic acid (loxoprofen, 192 mg, 0.78 mmol) and 1-(3-dimethylaminopropyl)-3-ethylcarbodiimide hydrochloride (EDCI, 173 mg, 0.9 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 2:1) to give the title compound as a light yellow solid, 0.26 g, 81.1% yield. MS (m/z): [M+H]⁺ calcd for C₃₂H₃₄N₆O₂, 535.27; found, 535.4. ¹H NMR (400 MHz, DMSO-d₆) δ 8.97 (s, 1H), 8.87 (d, J=1.6 Hz, 1H), 8.40 (s, 1H), 8.11 (d, J=4.2 Hz, 1H), 7.34 (d, J=7.8 Hz, 2H), 7.30 (d, J=4.2 Hz, 1H), 7.11 (d, J=7.8 Hz, 2H), 6.00 (q, J=6.8 Hz, 1H), 4.55 (td, J=9.6, 4.2 Hz, 1H), 3.31-3.15 (m, 2H), 2.90 (dd, J=13.2, 3.4 Hz, 1H), 2.38 (tdd, J=20.0, 14.0, 5.9 Hz, 3H), 2.20 (ddd, J=18.1, 8.2, 2.8 Hz, 1H), 2.05 (dd, J=10.1, 8.6 Hz, 1H), 1.92-1.75 (m, 3H), 1.68-1.50 (m, 7H), 1.48-1.22 (m, 5H)

### Example 78

### (R)—N-(4-(2-(4-(1-(2-cyano-1-cyclopentylethyl)-1H-pyrazol-4-yl)-7H-pyrrolo [2, 3-d] pyrimidin-7-yl)-2-oxoethyl) phenyl) acetamide

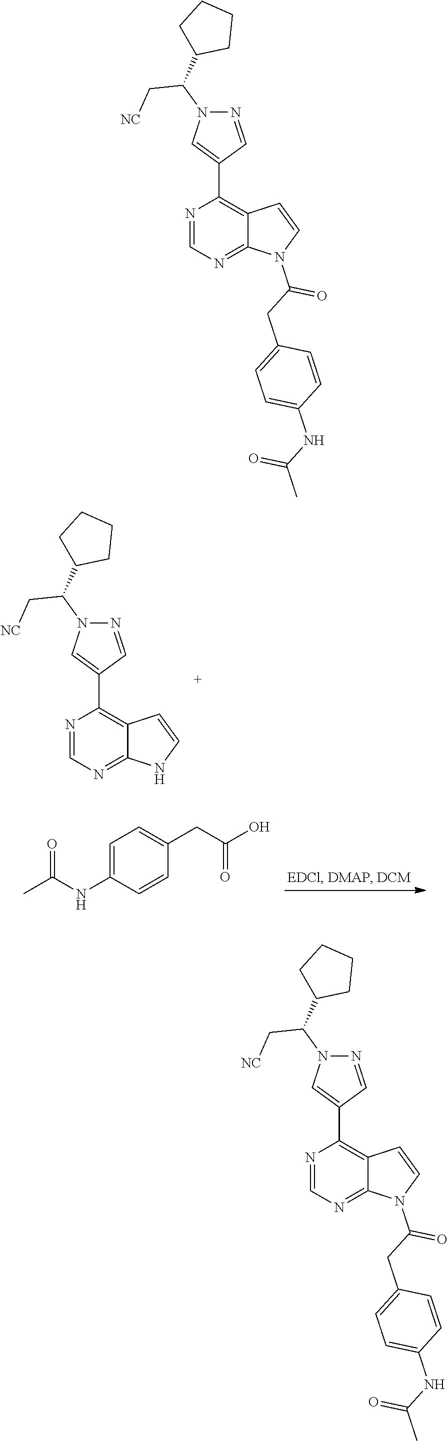 [SMILES: CC(=O)Nc1ccc(CC(=O)n2ccc3c(-c4cnn([C@H](CC#N)C5CCCC5)c4)ncnc32)cc1 ; N#CC[C@H](C1CCCC1)n1cc(-c2ncnc3[nH]ccc23)cn1 ; CC(=O)Nc1ccc(CC(=O)O)cc1 ; CC(=O)Nc1ccc(CC(=O)n2ccc3c(-c4cnn([C@H](CC#N)C5CCCC5)c4)ncnc32)cc1]

### Synthesis of (R)—N-(4-(2-(4-(1-(2-cyano-1-cyclopentylethyl)-1H-pyrazol-4-yl)-7H-pyrrolo [2, 3-d] pyrimidin-7-yl)-2-oxoethyl) phenyl) acetamide

(R)-3-(4-(7h-pyrrolo [2, 3-d] pyridin-4-yl)-1h-pyrazol-1-yl)-3-cyclopentylpropane (Ruxolitinib, 184 mg, 0.6 mmol), 4-dimethylamino pyridine (DMAP, 7 mg, 0.06 mmol), 2-(4-acetamidophenyl) acetic acid (actarit, 151 mg, 0.78 mmol) and 1-(3-dimethylaminopropyl)-3-ethylcarbodiimide hydrochloride (EDCI, 173 mg, 0.9 mmol) were dissolved in dichloromethane (10 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 2:3) to give the title compound as a light yellow solid, 0.18 g, 62.3% yield. MS (m/z): [M+H]⁺ calcd for C₂₇H₂₇N₇O₂, 482.22; found, 482.3. ¹H NMR (400 MHz, DMSO-d₆) δ 9.90 (s, 1H), 8.97 (s, 1H), 8.90 (s, 1H), 8.43 (s, 1H), 8.12 (d, J=4.2 Hz, 1H), 7.54 (d, J=8.1 Hz, 2H), 7.32 (dd, J=16.1, 6.1 Hz, 3H), 4.87 (s, 2H), 4.55 (td, J=9.6, 4.1 Hz, 1H), 3.30-3.17 (m, 2H), 2.43 (p, J=8.5 Hz, 1H), 2.04 (s, 3H), 1.83 (dhept, J=12.4, 4.1, 3.7 Hz, 1H), 1.67-1.41 (m, 4H), 1.39-1.16 (m, 3H).

### Example 79

### (R)-3-(4-(7-(2-(1-(4-chlorobenzoyl)-5-methoxy-2-methyl-1H-indol-3-yl) acetyl)-7H-pyrrolo [2, 3-d] pyrimidin-4-yl)-1H-pyrazol-1-yl)-3-cyclopentylpropanenitrile

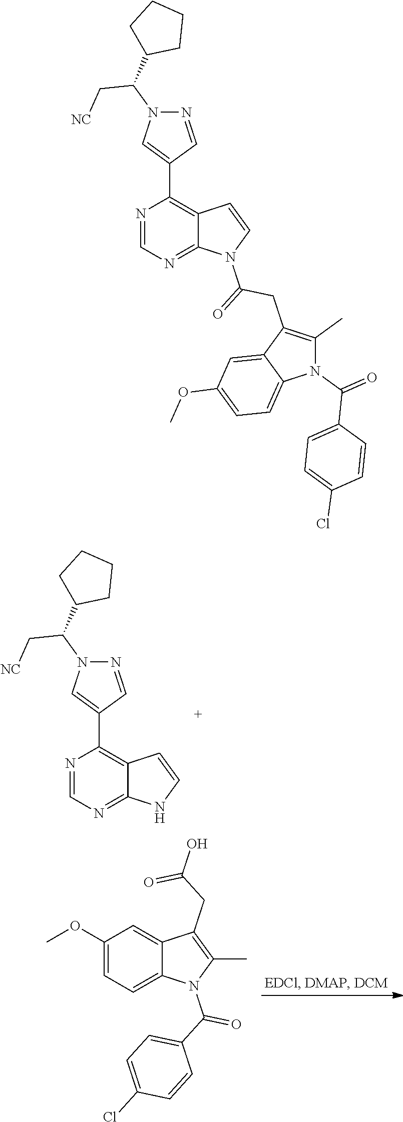

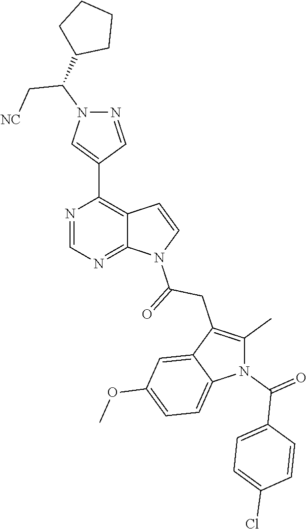 [SMILES: COc1ccc2c(c1)c(CC(=O)n1ccc3c(-c4cnn([C@H](CC#N)C5CCCC5)c4)ncnc31)c(C)n2C(=O)c1ccc(Cl)cc1]

### Synthesis of (R)-3-(4-(7-(2-(1-(4-chlorobenzoyl)-5-methoxy-2-methyl-1H-indol-3-yl) acetyl)-7H-pyrrolo [2, 3-d] pyrimidin-4-yl)-1H-pyrazol-1-yl)-3-cyclopentylpropanenitrile

(R)-3-(4-(7h-pyrrolo [2, 3-d] pyridin-4-yl)-1H-pyrazol-1-yl)-3-cyclopentylpropane (Ruxolitinib, 184 mg, 0.6 mmol), 4-dimethylamino pyridine (DMAP, 7 mg, 0.06 mmol), 1-(4-chlorobenzoyl)-5-methoxy-2-methyl-1H-indole-3-acetic acid (indomethacin, 279 mg, 0.78 mmol), and 1-(3-dimethylaminopropyl)-3-ethylcarbodiimide hydrochloride (EDCI, 173 mg, 0.9 mmol) was dissolved in dichloromethane (10 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 2:1) to give the title compound as a light yellow solid, 0.3 g, 77.5% yield. MS (m/z): [M+H]⁺ calcd for C₃₆H₃₂ClN₇O₃, 646.23; found, 646.1. ¹H NMR (400 MHz, DMSO-d₆) δ 9.03 (s, 1H), 8.93 (s, 1H), 8.47 (s, 1H), 8.16 (d, J=4.2 Hz, 1H), 7.76-7.63 (m, 4H), 7.38 (d, J=4.2 Hz, 1H), 7.18 (d, J=2.5 Hz, 1H), 7.00 (d, J=9.0 Hz, 1H), 6.73 (dd, J=9.1, 2.6 Hz, 1H), 5.05 (s, 2H), 4.58 (td, J=9.6, 4.3 Hz, 1H), 3.71 (s, 3H), 3.31-3.19 (m, 2H), 2.46 (q, J=8.2 Hz, 1H), 2.28 (s, 3H), 1.90-1.80 (m, 1H), 1.68-1.31 (m, 7H).

### Example 80

### Tert-butyl ((S)-2-(4-(1-((R)-2-cyano-1-cyclopentylethyl)-1H-pyrazol-4-yl)-7H-pyrrolo [2, 3-d] pyrimidin-7-yl)-2-oxo-1-phenylethyl) carbamate

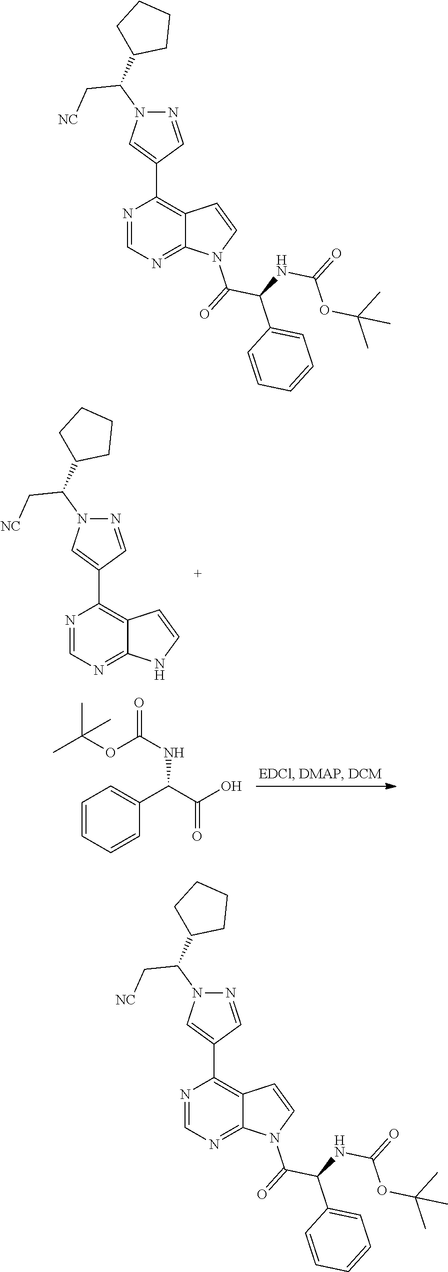

### Synthesis of tert-butyl ((S)-2-(4-(1-((R)-2-cyano-1-cyclopentylethyl)-1H-pyrazol-4-yl)-7H-pyrrolo [2, 3-d] pyrimidin-7-yl)-2-oxo-1-phenylethyl) carbamate

(R)-3-(4-(7h-pyrrolo [2, 3-d] pyridin-4-yl)-1h-pyrazol-1-yl)-3-cyclopentylpropane (Ruxolitinib, 184 mg, 0.6 mmol), 4-dimethylamino pyridine (DMAP, 7 mg, 0.06 mmol), N-Boc-L-phenylglycine (196 mg, 0.78 mmol) and 1-(3-dimethylaminopropyl)-3-ethylcarbodiimide hydrochloride (EDCI, 173 mg, 0.9 mmol) were dissolved in dichloromethane (10 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 2:1) to give the title compound as a yellow solid, 0.26 g, 55.6% yield. MS (m/z): [M+H]⁺ calcd for C₃₀H₃₃N₇O₃, 540.26; found, 540.3. ¹H NMR (400 MHz, DMSO-d₆) δ 8.97 (s, 1H), 8.87 (s, 1H), 8.41 (s, 1H), 8.14 (d, J=4.2 Hz, 1H), 7.95 (d, J=7.6 Hz, 1H), 7.52 (d, J=7.3 Hz, 2H), 7.42 (d, J=7.7 Hz, 1H), 7.31 (td, J=11.8, 10.6, 5.4 Hz, 4H), 4.54 (td, J=9.6, 4.3 Hz, 1H), 3.29-3.12 (m, 2H), 2.43 (h, J=8.5 Hz, 1H), 1.82 (dtd, J=11.9, 7.4, 4.2 Hz, 1H), 1.65-1.50 (m, 3H), 1.46-1.23 (m, 13H).

### Example 81

### Methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-((3-chloro-2-methylphenyl) amino) benzoate

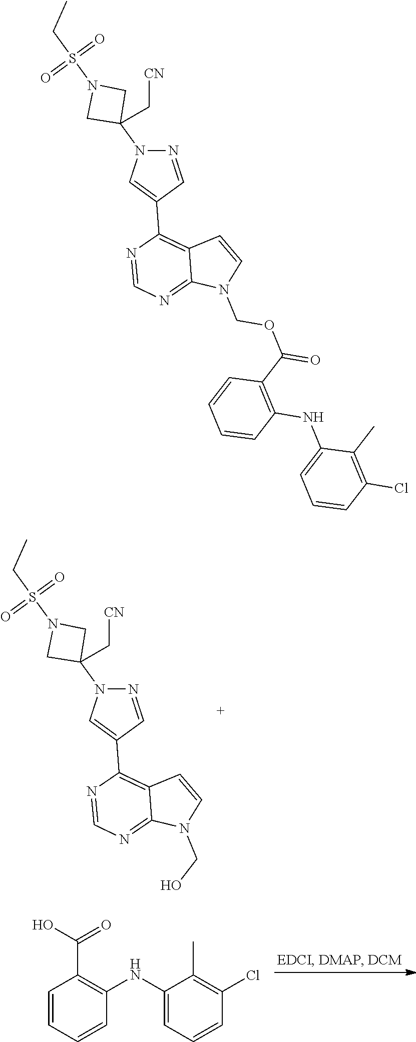 [SMILES: CCS(=O)(=O)N1CC(CC#N)(n2cc(-c3ncnc4c3ccn4COC(=O)c3ccccc3Nc3cccc(Cl)c3C)cn2)C1 ; CCS(=O)(=O)N1CC(CC#N)(n2cc(-c3ncnc4c3ccn4CO)cn2)C1 ; Cc1c(Cl)cccc1Nc1ccccc1C(=O)O]

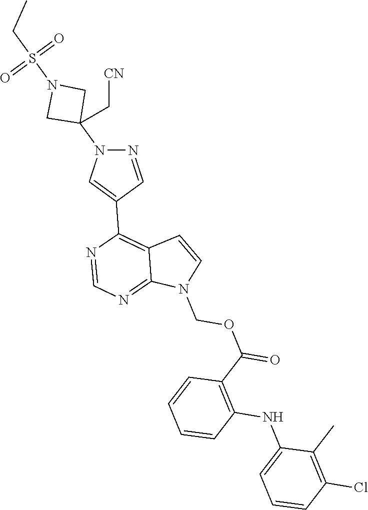 [SMILES: CCS(=O)(=O)N1CC(CC#N)(n2cc(-c3ncnc4c3ccn4COC(=O)c3ccccc3Nc3cccc(Cl)c3C)cn2)C1]

### Synthesis of methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-((3-chloro-2-methylphenyl) amino) benzoate

2-(1-(Ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile (201 mg, 0.5 mmol), 4-dimethylamino pyridine (DMAP, 31 mg, 0.25 mmol), 2-((3-chloro-2-methylphenyl) amino) benzoic acid (tolfenamic acid, 137 mg, 0.53 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:2) to give the title compound as a yellow solid, 0.26 g, 80.7% yield. MS (m/z): [M+H]⁺ calcd for C₃₁H₂₉ClN₈O₄S, 645.17; found, 645.1. ¹H NMR (400 MHz, DMSO-d₆) δ 9.12 (s, 1H), 8.98 (s, 1H), 8.84 (s, 1H), 8.52 (s, 1H), 7.93 (d, J=3.8 Hz, 1H), 7.79 (dd, J=8.4, 1.7 Hz, 1H), 7.37 (td, J=7.6, 7.1, 1.7 Hz, 1H), 7.33-7.21 (m, 4H), 6.78-6.70 (m, 2H), 6.55 (s, 2H), 4.62 (d, J=9.1 Hz, 2H), 4.26 (d, J=9.1 Hz, 2H), 3.71 (s, 2H), 3.24 (q, J=7.3 Hz, 2H), 2.24 (s, 3H), 1.26 (t, J=7.3 Hz, 3H).

### Example 82

### (R)-3-cyclopentyl-3-(4-(7-(2-((2, 3-dimethylphenyl) amino) benzoyl)-7H-pyrrolo [2, 3-d] pyrimidin-4-yl)-1H-pyrazol-1-yl) propanenitrile

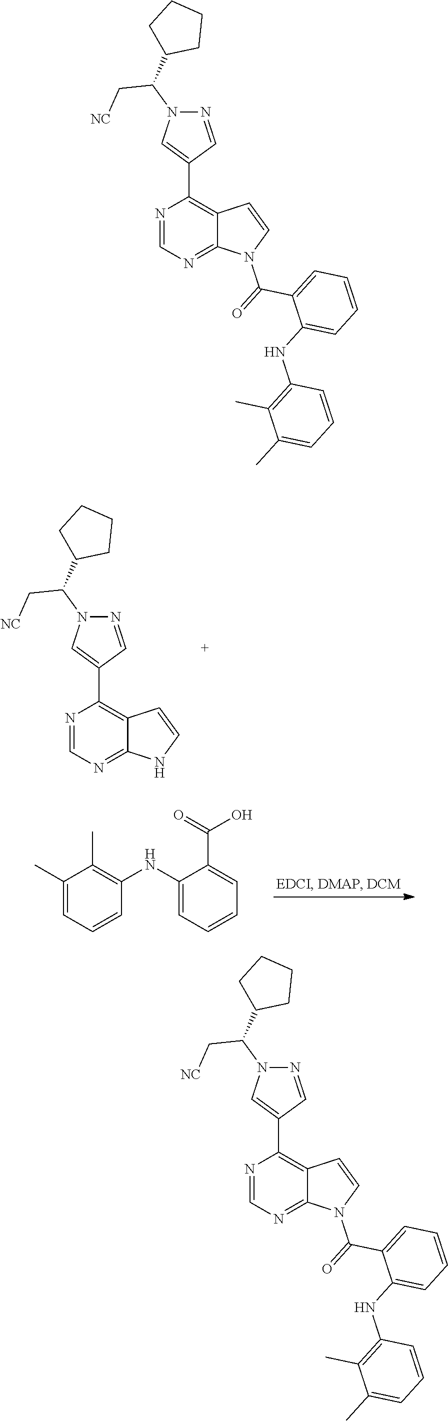 [SMILES: Cc1cccc(Nc2ccccc2C(=O)n2ccc3c(-c4cnn([C@H](CC#N)C5CCCC5)c4)ncnc32)c1C ; N#CC[C@H](C1CCCC1)n1cc(-c2ncnc3[nH]ccc23)cn1 ; Cc1cccc(Nc2ccccc2C(=O)O)c1C ; Cc1cccc(Nc2ccccc2C(=O)n2ccc3c(-c4cnn([C@H](CC#N)C5CCCC5)c4)ncnc32)c1C]

### Synthesis of (R)-3-cyclopentyl-3-(4-(7-(2-((2, 3-dimethylphenyl) amino) benzoyl)-7H-pyrrolo [2, 3-d] pyrimidin-4-yl)-1H-pyrazol-1-yl) propanenitrile

(R)-3-(4-(7h-pyrrolo [2, 3-d] pyridin-4-yl)-1h-pyrazol-1-yl)-3-cyclopentylpropane (Ruxolitinib, 184 mg, 0.6 mmol), 4-dimethylamino pyridine (DMAP, 7 mg, 0.06 mmol), 2-((2, 3-dimethylphenyl) amino) benzoic acid (mefenamic acid, 188 mg, 0.78 mmol) and 1-(3-dimethylaminopropyl)-3-ethylcarbodiimide hydrochloride (EDCI, 173 mg, 0.9 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=4:1 to 3:1) to give the title compound as a yellow solid, 0.08 g, 25.2% yield. MS (m/z): [M+H]⁺ calcd for C₃₂H₃₁N₇O, 530.26; found, 530.3. ¹H NMR (400 MHz, DMSO-d₆) δ 8.88 (s, 1H), 8.73 (s, 1H), 8.44 (d, J=13.9 Hz, 2H), 7.87 (d, J=4.0 Hz, 1H), 7.42 (ddd, J=7.7, 4.5, 2.8 Hz, 2H), 7.29 (d, J=4.1 Hz, 1H), 7.08-7.01 (m, 2H), 6.94 (dd, J=6.2, 2.6 Hz, 1H), 6.87-6.75 (m, 2H), 4.56 (td, J=9.7, 4.2 Hz, 1H), 3.29-3.15 (m, 2H), 2.45 (q, J=8.4 Hz, 1H), 2.22 (s, 3H), 1.98 (s, 3H), 1.83 (dtd, J=12.3, 7.4, 3.8 Hz, 1H), 1.55 (tdt, J=32.5, 29.7, 18.3, 6.6 Hz, 4H), 1.39-1.22 (m, 3H).

### Example 83

### Methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-((2, 3-dimethylphenyl) amino) benzoate

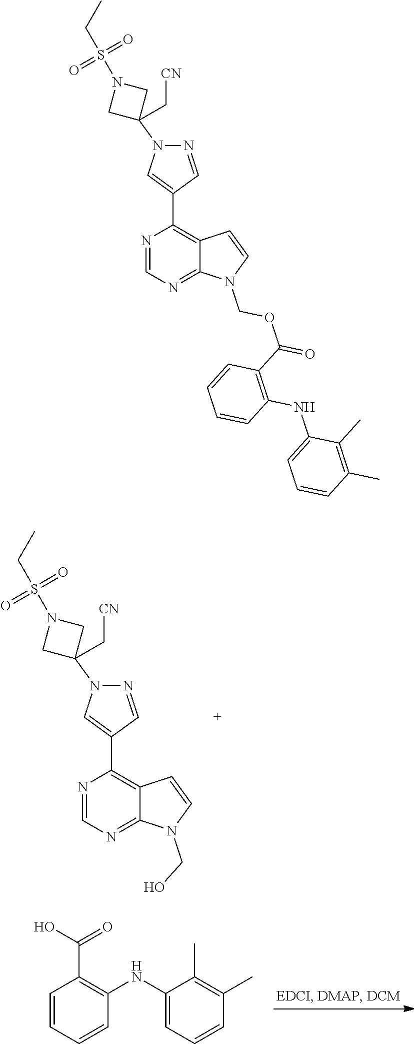 [SMILES: CCS(=O)(=O)N1CC(CC#N)(n2cc(-c3ncnc4c3ccn4COC(=O)c3ccccc3Nc3cccc(C)c3C)cn2)C1 ; CCS(=O)(=O)N1CC(CC#N)(n2cc(-c3ncnc4c3ccn4CO)cn2)C1 ; Cc1cccc(Nc2ccccc2C(=O)O)c1C]

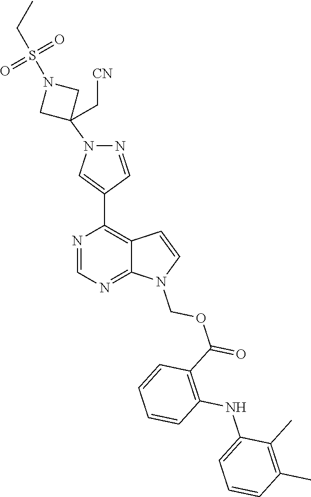 [SMILES: CCS(=O)(=O)N1CC(CC#N)(n2cc(-c3ncnc4c3ccn4COC(=O)c3ccccc3Nc3cccc(C)c3C)cn2)C1]

### Synthesis of methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-((2, 3-dimethylphenyl) amino) benzoate

2-(1-(ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile (201 mg, 0.5 mmol), 4-dimethylamino pyridine (DMAP, 31 mg, 0.25 mmol), 22-((2, 3-dimethylphenyl) amino) benzoic acid (mefenamic acid, 127 mg, 0.53 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:2) to give the title compound as a yellow solid, 0.27 g, 86.5% yield. MS (m/z): [M+H]⁺ calcd for C₃₂H₃₂N₈O₄S, 625.23; found, 625.2. ¹H NMR (400 MHz, DMSO-d₆) δ 9.06 (s, 1H), 8.98 (s, 1H), 8.85 (s, 1H), 8.52 (s, 1H), 7.94 (d, J=3.8 Hz, 1H), 7.76 (dd, J=8.1, 1.7 Hz, 1H), 7.32 (ddd, J=8.6, 7.0, 1.7 Hz, 1H), 7.27 (d, J=3.8 Hz, 1H), 7.18-7.05 (m, 3H), 6.70-6.59 (m, 2H), 6.55 (s, 2H), 4.61 (d, J=9.1 Hz, 2H), 4.25 (d, J=9.1 Hz, 2H), 3.70 (s, 2H), 3.24 (q, J=7.3 Hz, 2H), 2.30 (s, 3H), 2.09 (s, 3H), 1.25 (t, J=7.3 Hz, 3H).

### Example 84

### Methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-(4-acetamidophenyl) acetate

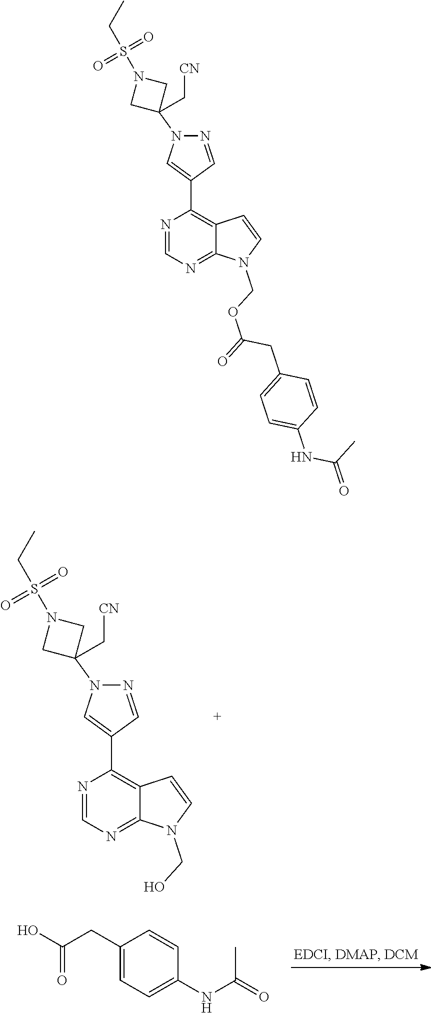 [SMILES: CCS(=O)(=O)N1CC(CC#N)(n2cc(-c3ncnc4c3ccn4COC(=O)Cc3ccc(NC(C)=O)cc3)cn2)C1 ; CCS(=O)(=O)N1CC(CC#N)(n2cc(-c3ncnc4c3ccn4CO)cn2)C1 ; CC(=O)Nc1ccc(CC(=O)O)cc1]

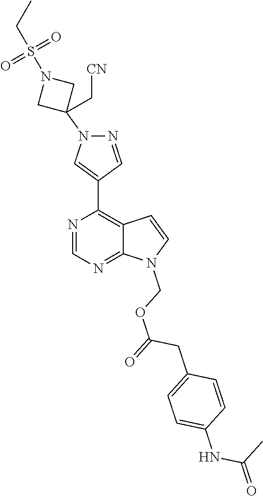 [SMILES: CCS(=O)(=O)N1CC(CC#N)(n2cc(-c3ncnc4c3ccn4COC(=O)Cc3ccc(NC(C)=O)cc3)cn2)C1]

### Synthesis of methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-(4-acetamidophenyl) acetate

2-(1-(Ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile (201 mg, 0.5 mmol), 4-dimethylamino pyridine (DMAP, 31 mg, 0.25 mmol), 2-(4-acetamidophenyl) acetic acid (actarit, 116 mg, 0.6 mmol), and 1-(3-dimethylaminopropyl)-3-ethylcarbodiimide hydrochloride (EDCI, 144 mg, 0.75 mmol) were dissolved in dichloromethane (10 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to give the title compound as a white solid, 0.22 g, 76.3% yield. MS (m/z): [M+H]⁺ calcd for C₂₇H₂₈N₈O₅S, 577.19; found, 577.2. ¹H NMR (400 MHz, DMSO-d₆) δ 9.89 (s, 1H), 8.98 (s, 1H), 8.84 (s, 1H), 8.52 (s, 1H), 7.78 (d, J=3.8 Hz, 1H), 7.49 (d, J=8.0 Hz, 2H), 7.19 (dd, J=31.5, 5.9 Hz, 3H), 6.29 (s, 2H), 4.63 (d, J=9.1 Hz, 2H), 4.27 (d, J=9.1 Hz, 2H), 3.69 (d, J=20.8 Hz, 4H), 3.26 (q, J=7.3 Hz, 2H), 2.04 (s, 3H), 1.27 (t, J=7.4 Hz, 3H).

### Example 85

### Methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-(1, 8-diethyl-1, 3, 4, 9-tetrahydropyrano [3, 4-b] indol-1-yl) acetate

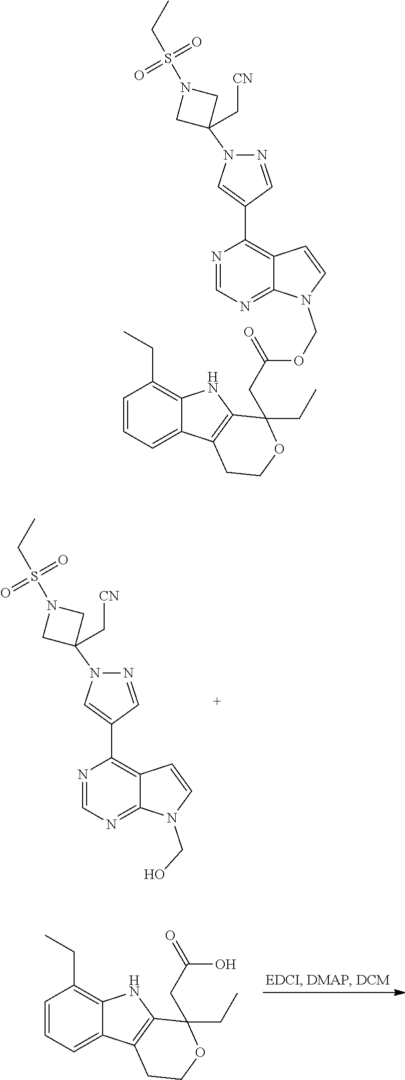 [SMILES: CCc1cccc2c3c([nH]c12)C(CC)(CC(=O)OCn1ccc2c(-c4cnn(C5(CC#N)CN(S(=O)(=O)CC)C5)c4)ncnc21)OCC3 ; CCS(=O)(=O)N1CC(CC#N)(n2cc(-c3ncnc4c3ccn4CO)cn2)C1 ; CCc1cccc2c3c([nH]c12)C(CC)(CC(=O)O)OCC3]

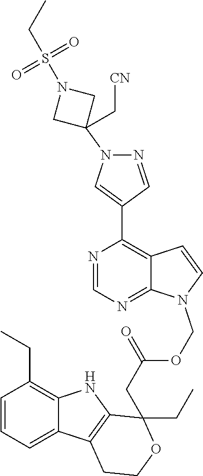 [SMILES: CCc1cccc2c3c([nH]c12)C(CC)(CC(=O)OCn1ccc2c(-c4cnn(C5(CC#N)CN(S(=O)(=O)CC)C5)c4)ncnc21)OCC3]

### Synthesis of methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-(1, 8-diethyl-1, 3, 4, 9-tetrahydropyrano [3, 4-b] indol-1-yl) acetate

2-(1-(Ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile (201 mg, 0.5 mmol), 4-dimethylamino pyridine (DMAP, 31 mg, 0.25 mmol), 1, 8-diethyl-1, 3, 4, 9-tetrahydropyran [3, 4-b] indole-1-acetic acid (etodolac, 172 mg, 0.6 mmol), and 1-(3-dimethylaminopropyl)-3-ethylcarbodiimide hydrochloride (EDCI, 144 mg, 0.75 mmol) were dissolved in dichloromethane (10 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=5:1 to 1:2) to give the title compound as a white solid, 0.17 g, 50.7% yield. MS (m/z): [M+H]⁺ calcd for C₃₄H₃₈N₈O₅S, 671.27; found, 671.2. ¹H NMR (400 MHz, DMSO-d₆) δ 10.42 (s, 1H), 8.97 (s, 1H), 8.80 (s, 1H), 8.51 (s, 1H), 7.61 (d, J=3.8 Hz, 1H), 7.20-7.10 (m, 2H), 6.93-6.81 (m, 2H), 6.27-6.12 (m, 2H), 4.63 (d, J=9.1 Hz, 2H), 4.27 (d, J=9.1 Hz, 2H), 3.88-3.76 (m, 2H), 3.72 (s, 2H), 3.25 (q, J=7.3 Hz, 2H), 3.06 (d, J=13.6 Hz, 1H), 2.86 (d, J=13.6 Hz, 1H), 2.81-2.75 (m, 2H), 2.61-2.48 (m, 2H), 2.01-1.80 (m, 2H), 1.30-1.21 (m, 6H), 0.58 (t, J=7.3 Hz, 3H).

### Example 86

### (4-(1-((R)-2-cyano-1-cyclopentylethyl)-1H-pyrazol-4-yl)-7H-pyrrolo [2, 3-d] pyrimidin-7-yl) methyl (S)-2-(4-isobutylphenyl) propanoate

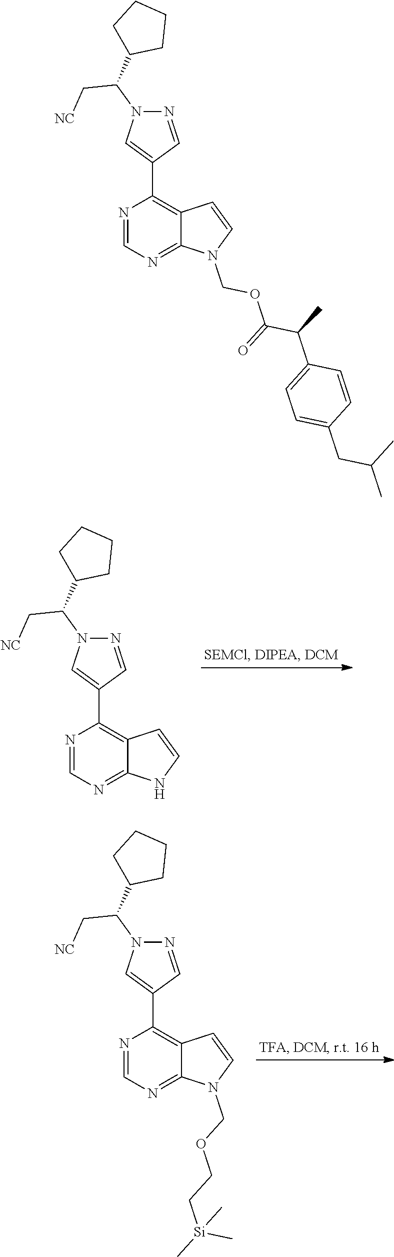 [SMILES: CC(C)Cc1ccc([C@H](C)C(=O)OCn2ccc3c(-c4cnn([C@H](CC#N)C5CCCC5)c4)ncnc32)cc1 ; N#CC[C@H](C1CCCC1)n1cc(-c2ncnc3[nH]ccc23)cn1 ; C[Si](C)(C)CCOCn1ccc2c(-c3cnn([C@H](CC#N)C4CCCC4)c3)ncnc21]

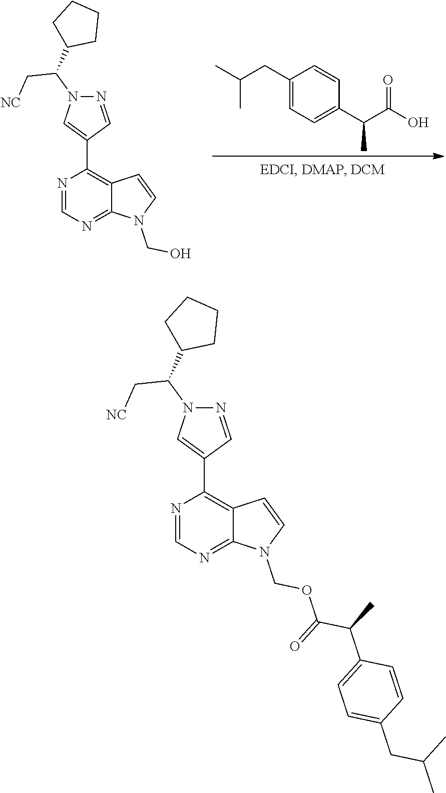 [SMILES: N#CC[C@H](C1CCCC1)n1cc(-c2ncnc3c2ccn3CO)cn1 ; CC(C)Cc1ccc([C@H](C)C(=O)O)cc1 ; CC(C)Cc1ccc([C@H](C)C(=O)OCn2ccc3c(-c4cnn([C@H](CC#N)C5CCCC5)c4)ncnc32)cc1]

### First Step: Synthesis of (R)-3-cyclopentyl-3-(4-(7-((2-(trimethylsilyl) ethoxy) methyl)-7H-pyrrolo [2, 3-d] pyrimidin-4-yl)-1H-pyrazol-1-yl) propanenitrile

(R)-3-(4-(7h-pyrrolo [2, 3-d] pyridin-4-yl)-1h-pyrazol-1-yl)-3-cyclopentylpropane (Ruxolitinib, 1000 mg, 3.26 mmol) and N, N-diisopropylethyl amine (0.7 mL, 4.9 mmoL) were added in dichloromethane (8 mL) under nitrogen. After stirring at room temperature for half an hour, (2-(chloromethoxy) ethyl) trimethylsilane (0.65 g, 3.92 mmol) was added in an ice-water bath, and stirring was continued at room temperature for 3 hours. The reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound 1.4 g, 98.5% yield. MS (m/z): [M+H]⁺ calcd for C₂₃H₃₂N₆OSi, 437.24; found, 437.3.

### Step 2: Synthesis of (R)-3-cyclopentyl-3-(4-(7-(hydroxymethyl)-7H-pyrrolo [2, 3-d] pyrimidin-4-yl)-1H-pyrazol-1-yl) propanenitrile

Trifluoroacetic acid (9 mL, 13.8 mg, 121 mmol) was slowly added dropwise to a solution of (R)-3-cyclopentyl-3-(4-(7-((2-(trimethylsilyl) ethoxy) methyl)-7H-pyrrolo [2, 3-d] pyrimidin-4-yl)-1H-pyrazol-1-yl) propanenitrile (1.4 g, 3.2 mmol) in dichloromethane (90 mL) under nitrogen in an ice-water bath. After half an hour, the ice-water bath was removed. The temperature was raised to room temperature and stirring was continued for an additional 2 hours. Saturated sodium bicarbonate solution was added to the above reaction solution at 0° C. to adjust the pH to 8. Then the mixture was poured into a separating funnel for separation. The organic layer was washed with a saturated salt water solution and dried over anhydrous sodium sulfate. After the filtration, the solvent was evaporated under reduced pressure to obtain the crude product. The crude product was further separated by silica gel column chromatography (dichloromethane/methanol=100:1 to 30:1) to give the title product 0.36 g, 33.4% yield. MS (m/z): [M+H]⁺ calcd for C₁₁H₂₀N₆O, 337.17; found, 337.3. ¹H NMR (400 MHz, DMSO-d6) δ 8.95-8.80 (m, 2H), 8.49 (s, 1H), 7.81 (d, J=3.7 Hz, 1H), 7.15 (d, J=3.7 Hz, 1H), 6.75 (t, J=7.3 Hz, 1H), 5.72 (d, J=7.3 Hz, 2H), 4.64 (td, J=9.6, 4.2 Hz, 1H), 3.42-3.26 (m, 2H), 2.53 (q, J=8.5 Hz, 1H), 1.92 (dtd, J=11.8, 7.5, 4.2 Hz, 1H), 1.76-1.50 (m, 4H), 1.50-1.23 (m, 3H).

### Step 3: Synthesis of (4-(1-((R)-2-cyano-1-cyclopentylethyl)-1H-pyrazol-4-yl)-7H-pyrrolo [2, 3-d] pyrimidin-7-yl) methyl (S)-2-(4-isobutylphenyl) propanoate

(R)-3-cyclopentyl-3-(4-(7-(hydroxymethyl)-7H-pyrrolo [2, 3-d] pyrimidin-4-yl)-1H-pyrazol-1-yl) propanenitrile (120 mg, 0.36 mmol), 4-dimethylamino pyridine (DMAP, 44 mg, 0.36 mmol), (S)-(+)-2-(4-isobutylphenyl) propanoic acid ((S)-(+)-ibuprofen, 110 mg, 0.54 mmol) and 1-(3-dimethylaminopropyl)-3-ethylcarbodiimide hydrochloride (EDCI, 103 mg, 0.54 mmol) were dissolved in a mixed solvent of dichloromethane (2.5 mL) and dimethylformamide (0.25 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.106 g, 56.1% yield. MS (m/z): [M+H]⁺ calcd for C₃₁H₃N₆O₂, 525.29; found, 525.3. ¹H NMR (400 MHz, Chloroform-d) δ 8.85 (s, 1H), 8.31 (d, J=10.9 Hz, 2H), 7.41 (d, J=3.8 Hz, 1H), 7.10 (d, J=8.1 Hz, 2H), 7.00 (d, J=7.9 Hz, 2H), 6.72 (d, J=3.8 Hz, 1H), 6.30-6.18 (m, 2H), 4.28 (ddd, J=10.1, 8.6, 4.0 Hz, 1H), 3.71 (q, J=7.1 Hz, 1H), 3.14 (dd, J=17.0, 8.6 Hz, 1H), 2.97 (dd, J=17.0, 4.0 Hz, 1H), 2.61 (ddd, J=16.8, 8.2, 4.6 Hz, 1H), 2.40 (d, J=7.1 Hz, 2H), 2.00-1.93 (m, 1H), 1.85-1.53 (m, 6H), 1.47 (d, J=7.2 Hz, 3H), 1.37-1.23 (m, 2H), 0.86 (d, J=6.6 Hz, 6H).

### Example 87

### (4-(1-((R)-2-cyano-1-cyclopentylethyl)-1H-pyrazol-4-yl)-7H-pyrrolo [2, 3-d] pyrimidin-7-yl) methyl (S)-2-(6-methoxynaphthalen-2-yl) propanoate

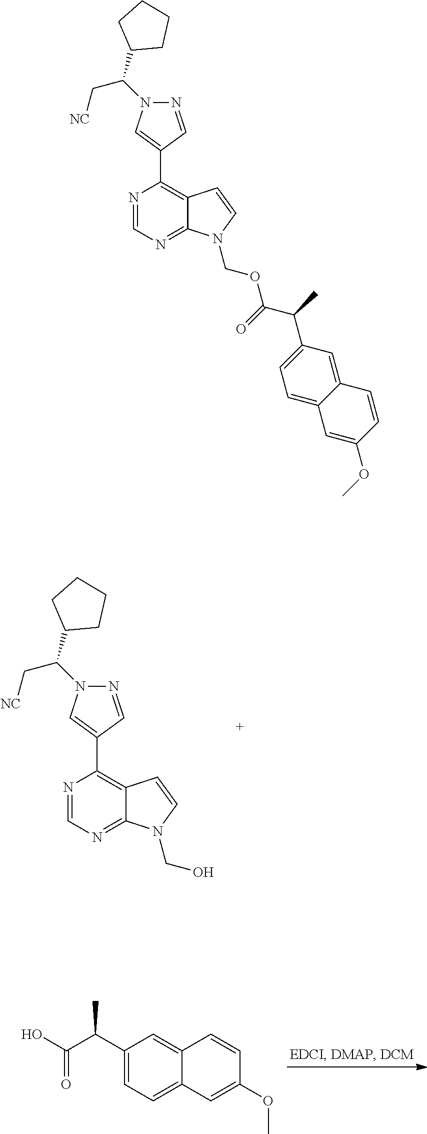 [SMILES: COc1ccc2cc([C@H](C)C(=O)OCn3ccc4c(-c5cnn([C@H](CC#N)C6CCCC6)c5)ncnc43)ccc2c1 ; N#CC[C@H](C1CCCC1)n1cc(-c2ncnc3c2ccn3CO)cn1 ; COc1ccc2cc([C@H](C)C(=O)O)ccc2c1]

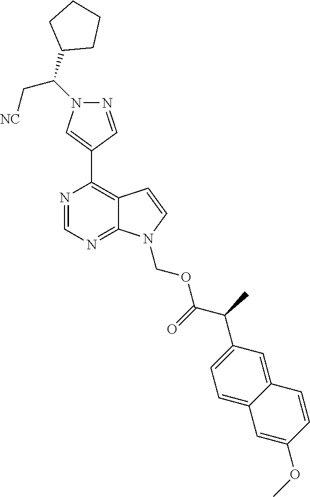 [SMILES: COc1ccc2cc([C@H](C)C(=O)OCn3ccc4c(-c5cnn([C@H](CC#N)C6CCCC6)c5)ncnc43)ccc2c1]

### Synthesis of (4-(1-((R)-2-cyano-1-cyclopentylethyl)-1H-pyrazol-4-yl)-7H-pyrrolo [2, 3-d] pyrimidin-7-yl) methyl (S)-2-(6-methoxynaphthalen-2-yl) propanoate

(R)-3-cyclopentyl-3-(4-(7-(hydroxymethyl)-7H-pyrrolo [2, 3-d] pyrimidin-4-yl)-1H-pyrazol-1-yl) propanenitrile (140 mg, 0.42 mmol), 4-dimethylamino pyridine (DMAP, 51 mg, 0.42 mmol), (S)-(+)-2-(6-methoxy-2-naphthyl) propanoic acid (naproxen, 144 mg, 0.63 mmol) and 1-(3-dimethylaminopropyl)-3-ethylcarbodiimide hydrochloride (EDCI, 120 mg, 0.63 mmol) were dissolved in dichloromethane (3 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.146 g, 63.4% yield. MS (m/z): [M+H]⁺ calcd for C₃₂H₃₂N₆O₃, 549.25; found, 549.3. ¹H NMR (400 MHz, Chloroform-d) δ 8.83 (s, 1H), 8.29 (d, J=7.0 Hz, 2H), 7.60 (t, J=8.6 Hz, 2H), 7.54 (d, J=1.8 Hz, 1H), 7.39 (d, J=3.8 Hz, 1H), 7.29 (dd, J=6.6, 1.9 Hz, 1H), 7.14-7.03 (m, 2H), 6.68 (d, J=3.8 Hz, 1H), 6.24 (d, J=2.4 Hz, 2H), 4.27 (ddd, J=10.1, 8.6, 4.0 Hz, 1H), 3.89 (s, 3H), 3.14 (dd, J=17.0, 8.6 Hz, 1H), 2.96 (dd, J=17.0, 4.0 Hz, 1H), 2.66-2.53 (m, 1H), 2.05-1.92 (m, 1H), 1.79-1.50 (m, 9H), 1.29 (td, J=12.8, 6.5 Hz, 2H).

### Example 88

### (4-(Methyl ((trans)-4-((N-methylsulfamoyl) methyl) cyclohexyl) amino)-7H-pyrrolo [2, 3-d] pyrimidin-7-yl) methyl (S)-2-(4-isobutylphenyl) propanoate

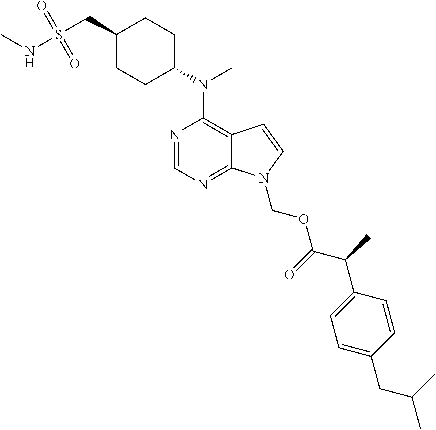 [SMILES: CNS(=O)(=O)C[C@H]1CC[C@H](N(C)c2ncnc3c2ccn3COC(=O)[C@@H](C)c2ccc(CC(C)C)cc2)CC1]

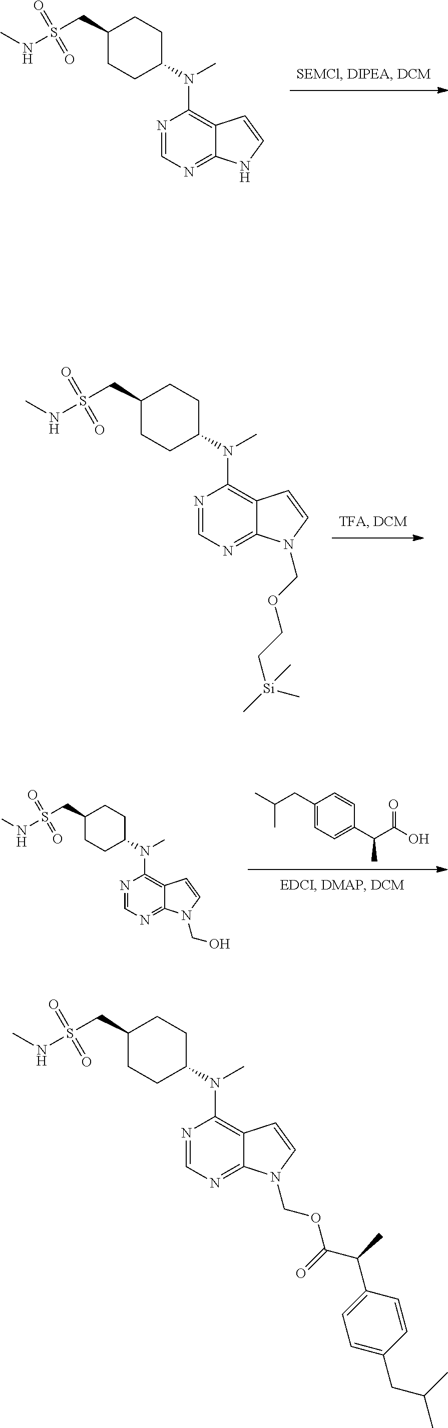 [SMILES: CNS(=O)(=O)C[C@H]1CC[C@H](N(C)c2ncnc3[nH]ccc23)CC1 ; CNS(=O)(=O)C[C@H]1CC[C@H](N(C)c2ncnc3c2ccn3COCC[Si](C)(C)C)CC1 ; CNS(=O)(=O)C[C@H]1CC[C@H](N(C)c2ncnc3c2ccn3CO)CC1 ; CC(C)Cc1ccc([C@H](C)C(=O)O)cc1 ; CNS(=O)(=O)C[C@H]1CC[C@H](N(C)c2ncnc3c2ccn3COC(=O)[C@@H](C)c2ccc(CC(C)C)cc2)CC1]

### First Step: Synthesis of N-methyl-1-((trans)-4-(methyl (7-((2-(trimethylsilyl) ethoxy) methyl)-7H-pyrrolo [2, 3-d] pyrimidin-4-yl) amino) cyclohexyl) methanesulfonamide

N-Methyl-1-((trans)-4-(methyl (7H-pyrrolo [2, 3-d] pyrimidin-4-yl) amino) cyclohexyl) methanesulfonamide (oclacitinib, 1687 mg, 5 mmol) and N, N-diisopropylethyl amine (780 mg, 6 mmol) were added in dichloromethane (50 mL) under nitrogen. After stirring at room temperature for half an hour, (2-(chloromethoxy) ethyl) trimethylsilane (1 g, 6 mmol) was added in an ice-water bath. Stirring was continued at room temperature overnight. After completion of the reaction, the reaction mixture was diluted with dichloromethane and washed with water and saturated salt water solution. The organic layer was dried over anhydrous sodium sulfate, and filtered. The solvent was evaporated under reduced pressure to give the title compound as an oily solid, 2 g, 85.6% yield. MS (m/z): [M+H]⁺ calcd for C₂₁H₃₇N₅O₃SSi, 468.24; found, 468.2.

### Step 2: Synthesis of 1-((trans)-4-((7-(hydroxymethyl)-7H-pyrrolo [2, 3-d] pyrimidin-4-yl) (methyl) amino) cyclohexyl)-N-methyl methanesulfonamide

Trifluoroacetic acid (6 mL, 9.2 g, 80.8 mmol) was slowly added dropwise to a solution of N-methyl-1-((trans)-4-(methyl (7-((2-(trimethylsilyl) ethoxy) methyl)-7H-pyrrolo [2, 3-d] pyrimidin-4-yl) amino) cyclohexyl) methanesulfonamide (2 g, 4.29 mmol) in dichloromethane (60 mL) under nitrogen in an ice-water bath. After half an hour, the ice-water bath was removed and the temperature was raised to room temperature and stirring was continued for 24 hours. Saturated sodium bicarbonate solution was added to the above reaction solution at 0° C. to adjust the pH to 8. Then the mixture was poured into a separating funnel for separation. The organic layer was washed with a saturated salt water solution and dried over anhydrous sodium sulfate. After the filtration, the solvent was evaporated under reduced pressure to obtain the crude product. The crude product was further separated by silica gel column chromatography (petroleum ether/ethyl acetate=1:1 to 0:1) to give the title product, 0.9 g, 98.5% yield. MS (m/z): [M+H]⁺ calcd for C₁₆H₂₅N₅O₃S, 368.17; found, 368.2.

### Step 3: Synthesis of (4-(methyl ((trans)-4-((N-methylsulfamoyl) methyl) cyclohexyl) amino)-7H-pyrrolo [2, 3-d] pyrimidin-7-yl) methyl (S)-2-(4-isobutylphenyl) propanoate

1-((Trans)-4-((7-(hydroxymethyl)-7H-pyrrolo [2, 3-d] pyrimidin-4-yl) (methyl) amino) cyclohexyl)-N-methyl methanesulfonamide (150 mg, 0.41 mmol), 4-dimethylaminopyridine (DMAP, 50 mg, 0.41 mmol), (S)-(+)-2-(4-isobutylphenyl) propanoic acid ((S)-(+)-ibuprofen, 126 mg, 0.61 mmol), and 1-(3-dimethylaminopropyl)-3-ethylcarbodiimide hydrochloride (EDCI, 117 mg, 0.61 mmol) were dissolved in dichloromethane (3 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=10:1 to 1:2) to give the title compound as a white solid, 0.086 g, 37.7% yield. MS (m/z): [M+H]⁺ calcd for C₂₉H₄₁N₅O₄S, 556.29; found, 556.3. ¹H NMR (400 MHz, Chloroform-d) δ 8.32 (s, 1H), 7.11 (d, J=7.7 Hz, 2H), 7.08-6.94 (m, 3H), 6.48 (d, J=3.8 Hz, 1H), 6.22-6.04 (m, 2H), 4.75 (s, 1H), 4.31 (q, J=5.3 Hz, 1H), 3.68 (q, J=7.2 Hz, 1H), 3.20 (s, 3H), 2.96 (d, J=6.2 Hz, 2H), 2.82 (d, J=5.1 Hz, 3H), 2.41 (d, J=7.1 Hz, 2H), 2.24-2.13 (m, 2H), 2.06-1.95 (m, 1H), 1.84 (ddd, J=22.7, 13.2, 5.3 Hz, 3H), 1.76-1.62 (m, 2H), 1.44 (d, J=7.3 Hz, 3H), 1.36 (dd, J=12.4, 9.1 Hz, 2H), 0.87 (d, J=6.6 Hz, 6H).

### Example 89

### (4-(Methyl ((trans)-4-((N-methylsulfamoyl) methyl) cyclohexyl) amino)-7H-pyrrolo [2, 3-d] pyrimidin-7-yl) methyl (S)-2-(6-methoxynaphthalen-2-yl) propanoate

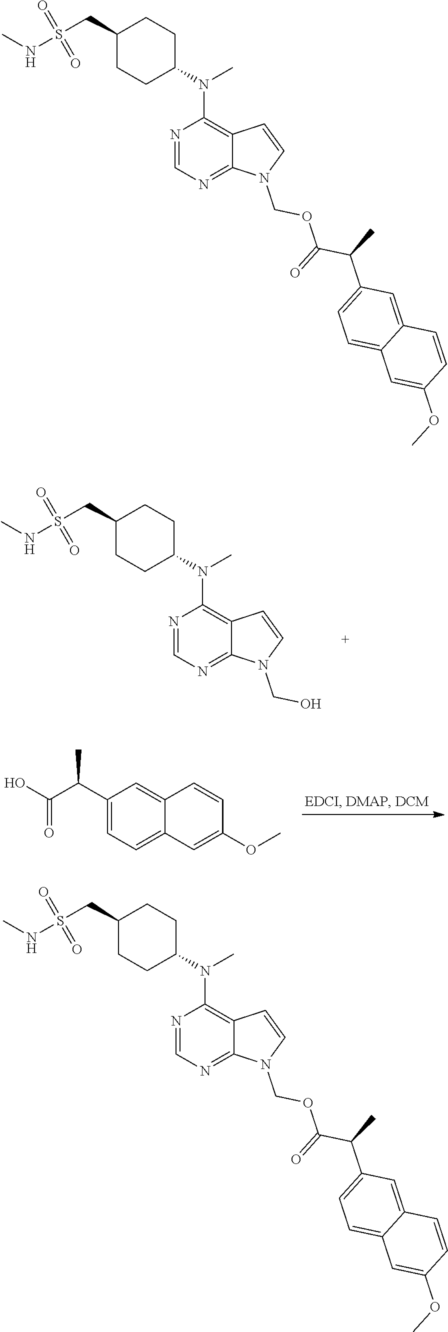 [SMILES: CNS(=O)(=O)C[C@H]1CC[C@H](N(C)c2ncnc3c2ccn3COC(=O)[C@@H](C)c2ccc3cc(OC)ccc3c2)CC1 ; CNS(=O)(=O)C[C@H]1CC[C@H](N(C)c2ncnc3c2ccn3CO)CC1 ; COc1ccc2cc([C@H](C)C(=O)O)ccc2c1 ; CNS(=O)(=O)C[C@H]1CC[C@H](N(C)c2ncnc3c2ccn3COC(=O)[C@@H](C)c2ccc3cc(OC)ccc3c2)CC1]

### Synthesis of (4-(methyl ((trans)-4-((N-methylsulfamoyl) methyl) cyclohexyl) amino)-7H-pyrrolo [2, 3-d] pyrimidin-7-yl) methyl (S)-2-(6-methoxynaphthalen-2-yl) propanoate

1-((Trans)-4-((7-(hydroxymethyl)-7H-pyrrolo [2, 3-d] pyrimidin-4-yl) (methyl) amino) cyclohexyl)-N-methyl methanesulfonamide (150 mg, 0.41 mmol), 4-dimethylamino pyridine (DMAP, 50 mg, 0.41 mmol), (S)-(+)-2-(6-methoxy-2-naphthyl) propanoic acid (naproxen, 141 mg, 0.61 mmol) and 1-(3-dimethylaminopropyl)-3-ethylcarbodiimide hydrochloride (EDCI, 117 mg, 0.61 mmol) were dissolved in dichloromethane (3 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=10:1 to 1:2) to give the title compound as a white solid, 0.056 g, 23.5% yield. MS (m/z): [M+H]⁺ calcd for C₃₀H₃₇N₅O₅S, 580.25; found, 580.3. ¹H NMR (400 MHz, Chloroform-d) δ 8.31 (s, 1H), 7.69-7.44 (m, 3H), 7.40-7.22 (m, 1H), 7.17-6.99 (m, 3H), 6.43 (d, J=3.8 Hz, 1H), 6.14 (q, J=10.6 Hz, 2H), 4.72 (s, 1H), 4.24 (q, J=5.4 Hz, 1H), 3.97-3.78 (m, 4H), 3.17 (s, 3H), 2.95 (d, J=6.2 Hz, 2H), 2.82 (d, J=5.3 Hz, 3H), 2.16 (d, J=13.1 Hz, 2H), 1.99 (s, 1H), 1.85 (d, J=11.1 Hz, 2H), 1.74-1.60 (m, 2H), 1.53 (d, J=7.2 Hz, 3H), 1.36 (td, J=13.7, 5.9 Hz, 2H).

### Example 90

### (4-(Methyl ((trans)-4-((N-methylsulfamoyl) methyl) cyclohexyl) amino)-7H-pyrrolo [2, 3-d] pyrimidin-7-yl) methyl 2-(3-benzoylphenyl) propanoate

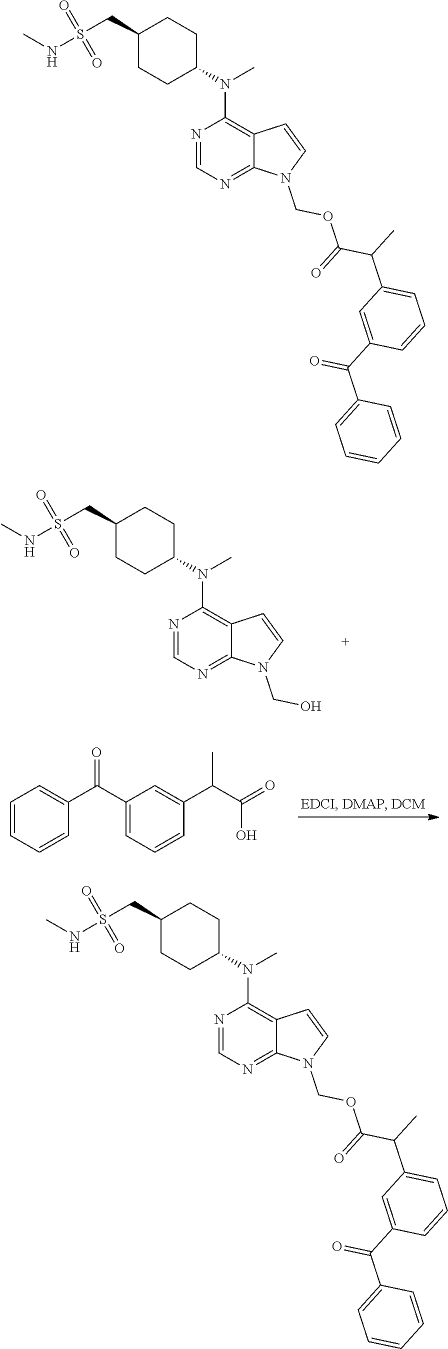 [SMILES: CNS(=O)(=O)C[C@H]1CC[C@H](N(C)c2ncnc3c2ccn3COC(=O)C(C)c2cccc(C(=O)c3ccccc3)c2)CC1 ; CNS(=O)(=O)C[C@H]1CC[C@H](N(C)c2ncnc3c2ccn3CO)CC1 ; CC(C(=O)O)c1cccc(C(=O)c2ccccc2)c1 ; CNS(=O)(=O)C[C@H]1CC[C@H](N(C)c2ncnc3c2ccn3COC(=O)C(C)c2cccc(C(=O)c3ccccc3)c2)CC1]

### Synthesis of (4-(methyl ((trans)-4-((N-methylsulfamoyl) methyl) cyclohexyl) amino)-7H-pyrrolo [2, 3-d] pyrimidin-7-yl) methyl 2-(3-benzoylphenyl) propanoate

1-((Trans)-4-((7-(hydroxymethyl)-7H-pyrrolo [2, 3-d] pyrimidin-4-yl) (methyl) amino) cyclohexyl)-N-methyl methanesulfonamide (220 mg, 0.6 mmol), 4-dimethylamino pyridine (DMAP, 7 mg, 0.06 mmol), 2-(3-benzoylphenyl) propanoic acid (ketoprofen, 198 mg, 0.78 mmol) and 1-(3-dimethylaminopropyl)-3-ethylcarbodiimide hydrochloride (EDCI, 173 mg, 0.9 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:2) to give the title compound as a white solid, 0.16 g, 44.2% yield. MS (m/z): [M+H]⁺ calcd for C₃₂H₃₇N₅O₅S, 604.25; found, 604.4. ¹H NMR (400 MHz, DMSO-d₆) δ 8.12 (s, 1H), 7.71-7.65 (m, 3H), 7.61-7.51 (m, 5H), 7.46 (t, J=8.0 Hz, 1H), 7.23 (d, J=3.8 Hz, 1H), 6.88 (q, J=4.9 Hz, 1H), 6.60 (d, J=3.7 Hz, 1H), 6.14 (s, 2H), 4.63 (s, 1H), 3.95 (q, J=7.1 Hz, 1H), 3.13 (s, 3H), 2.95 (d, J=6.2 Hz, 2H), 2.59 (d, J=5.0 Hz, 3H), 2.09-2.01 (m, 2H), 1.81 (s, 1H), 1.72-1.62 (m, 4H), 1.39 (d, J=7.1 Hz, 3H), 1.35-1.26 (m, 2H).

### Example 91

### (4-(Methyl ((trans)-4-((N-methylsulfamoyl) methyl) cyclohexyl) amino)-7H-pyrrolo [2, 3-d] pyrimidin-7-yl) methyl (S)-2-((tert-butoxycarbonyl) amino)-2-phenylacetate

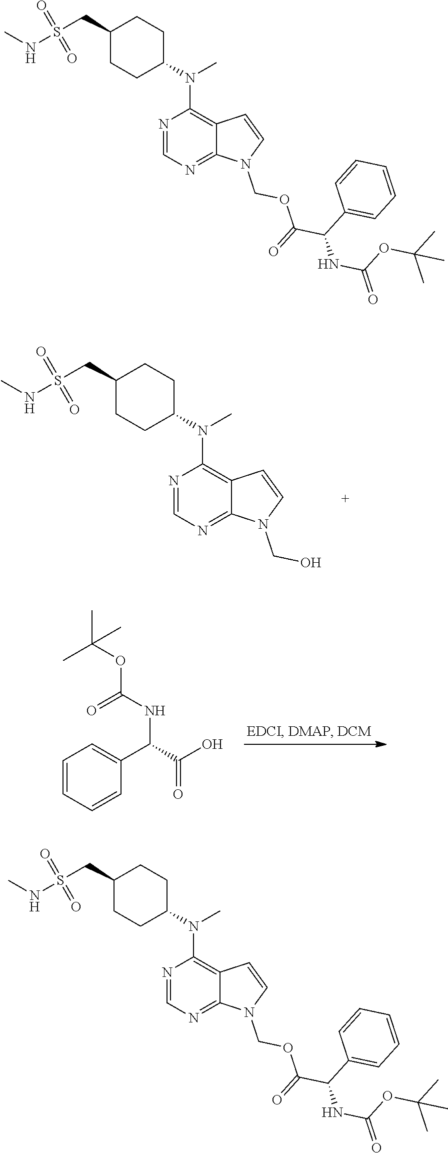

### Synthesis of (4-(methyl ((trans)-4-((N-methylsulfamoyl) methyl) cyclohexyl) amino)-7H-pyrrolo [2, 3-d] pyrimidin-7-yl) methyl (S)-2-((tert-butoxycarbonyl) amino)-2-phenylacetate

1-((Trans)-4-((7-(hydroxymethyl)-7H-pyrrolo [2, 3-d] pyrimidin-4-yl) (methyl) amino) cyclohexyl)-N-methyl methanesulfonamide (220 mg, 0.6 mmol), 4-dimethylaminopyridine (DMAP, 7 mg, 0.06 mmol), N-Boc-L-phenylglycine (196 mg, 0.78 mmol) and 1-(3-dimethylaminopropyl)-3-ethylcarbodiimide hydrochloride (EDCI, 173 mg, 0.9 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:2) to give the title compound as a white solid, 0.14 g, 38.8% yield. MS (m/z): [M+H]⁺ calcd for C₂₉H₄₀N₆O₆S, 601.27; found, 601.4. ¹H NMR (400 MHz, DMSO-d₆) δ 8.21 (s, 1H), 7.85 (d, J=8.0 Hz, 1H), 7.39-7.25 (m, 6H), 6.94 (q, J=5.0 Hz, 1H), 6.67 (d, J=3.8 Hz, 1H), 6.27-6.16 (m, 2H), 5.22 (d, J=7.9 Hz, 1H), 4.71 (s, 1H), 3.21 (s, 3H), 3.01 (d, J=6.2 Hz, 2H), 2.64 (d, J=5.0 Hz, 3H), 2.10 (d, J=12.7 Hz, 2H), 1.95-1.85 (m, 1H), 1.73 (d, J=7.5 Hz, 4H), 1.41 (s, 11H).

### Example 92

### (4-(Methyl ((trans)-4-((N-methylsulfamoyl) methyl) cyclohexyl) amino)-7H-pyrrolo [2, 3-d] pyrimidin-7-yl) methyl 2-(2-((2, 6-dichlorophenyl) amino) phenyl) acetate

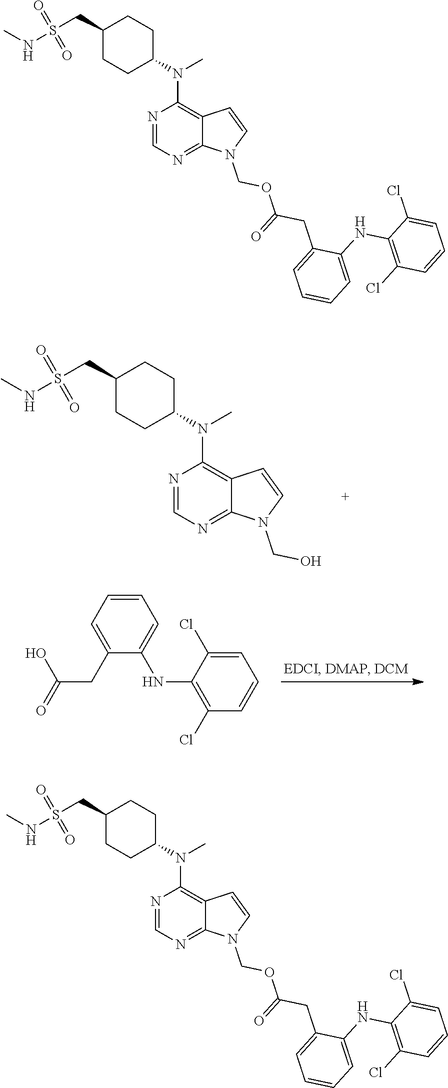 [SMILES: CNS(=O)(=O)C[C@H]1CC[C@H](N(C)c2ncnc3c2ccn3COC(=O)Cc2ccccc2Nc2c(Cl)cccc2Cl)CC1 ; CNS(=O)(=O)C[C@H]1CC[C@H](N(C)c2ncnc3c2ccn3CO)CC1 ; O=C(O)Cc1ccccc1Nc1c(Cl)cccc1Cl ; CNS(=O)(=O)C[C@H]1CC[C@H](N(C)c2ncnc3c2ccn3COC(=O)Cc2ccccc2Nc2c(Cl)cccc2Cl)CC1]

### Synthesis of (4-(methyl ((trans)-4-((N-methylsulfamoyl) methyl) cyclohexyl) amino)-7H-pyrrolo [2, 3-d] pyrimidin-7-yl) methyl 2-(2-((2, 6-dichlorophenyl) amino) phenyl) acetate

1-((Trans)-4-((7-(hydroxymethyl)-7H-pyrrolo [2, 3-d] pyrimidin-4-yl) (methyl) amino) cyclohexyl)-N-methyl methanesulfonamide (220 mg, 0.6 mmol), 4-dimethylamino pyridine (DMAP, 7 mg, 0.06 mmol), 2-(2, 6-dichloroanilino) phenylacetic acid (diclofenac, 444 mg, 1.5 mmol) and 1-(3-dimethylaminopropyl)-3-ethylcarbodiimide hydrochloride (EDCI, 173 mg, 0.9 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:1) to give the title compound as a white solid, 0.14 g, 36.2% yield. MS (m/z): [M+H]⁺ calcd for C₃₀H₃₄Cl₂N₆O₄S, 645.17; found, 645.3. ¹H NMR (400 MHz, DMSO-d₆) δ 8.17 (s, 1H), 7.52 (d, J=8.1 Hz, 2H), 7.30 (d, J=3.8 Hz, 1H), 7.24-7.12 (m, 2H), 7.05 (td, J=7.7, 1.6 Hz, 1H), 6.95 (s, 1H), 6.87 (q, J=4.9 Hz, 1H), 6.82 (td, J=7.4, 1.2 Hz, 1H), 6.64 (d, J=3.8 Hz, 1H), 6.23 (dd, J=8.0, 1.2 Hz, 1H), 6.19 (s, 2H), 4.66 (s, 1H), 3.82 (s, 2H), 3.16 (s, 3H), 2.95 (d, J=6.2 Hz, 2H), 2.59 (d, J=5.0 Hz, 3H), 2.05 (d, J=12.1 Hz, 2H), 1.82 (s, 1H), 1.75-1.65 (m, 4H), 1.30 (q, J=7.9, 7.2 Hz, 2H).

### Example 93

### (4-(Methyl ((trans)-4-((N-methylsulfamoyl) methyl) cyclohexyl) amino)-7H-pyrrolo [2, 3-d] pyrimidin-7-yl) methyl 2-(4-acetamidophenyl) acetate

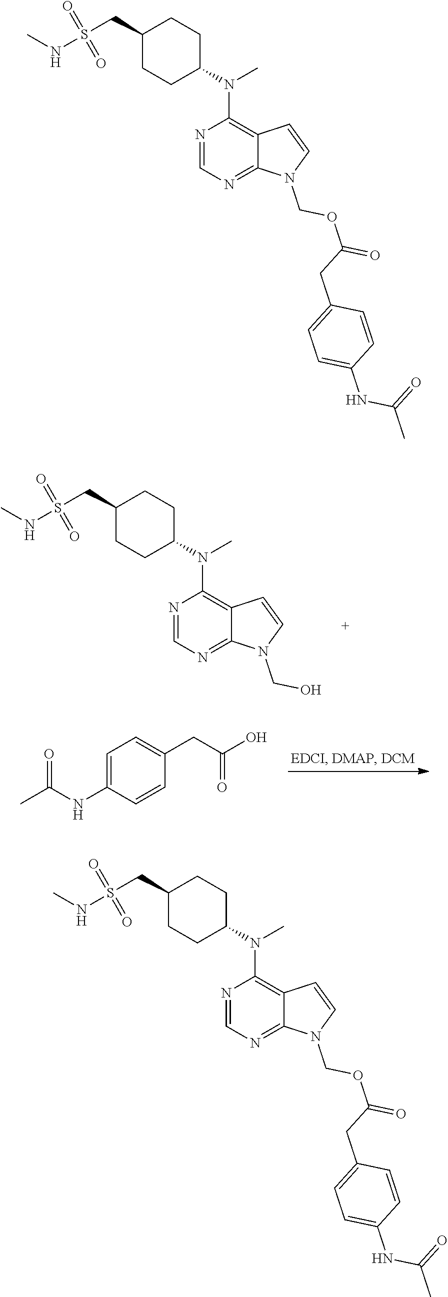 [SMILES: CNS(=O)(=O)C[C@H]1CC[C@H](N(C)c2ncnc3c2ccn3COC(=O)Cc2ccc(NC(C)=O)cc2)CC1 ; CNS(=O)(=O)C[C@H]1CC[C@H](N(C)c2ncnc3c2ccn3CO)CC1 ; CC(=O)Nc1ccc(CC(=O)O)cc1 ; CNS(=O)(=O)C[C@H]1CC[C@H](N(C)c2ncnc3c2ccn3COC(=O)Cc2ccc(NC(C)=O)cc2)CC1]

### Synthesis of (4-(methyl ((trans)-4-((N-methylsulfamoyl) methyl) cyclohexyl) amino)-7H-pyrrolo [2, 3-d] pyrimidin-7-yl) methyl 2-(4-acetamidophenyl) acetate

1-((Trans)-4-((7-(hydroxymethyl)-7H-pyrrolo [2, 3-d] pyrimidin-4-yl) (methyl) amino) cyclohexyl)-N-methyl methanesulfonamide (220 mg, 0.6 mmol), 4-dimethylaminopyridine (DMAP, 7 mg, 0.06 mmol), 2-(4-acetamidophenyl) acetic acid (actarit, 151 mg, 0.78 mmol), and 1-(3-dimethylaminopropyl)-3-ethylcarbodiimide hydrochloride (EDCI, 173 mg, 0.9 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 0:1) to give the title compound as a white solid, 0.016 g, 49.2% yield. MS (m/z): [M+H]⁺ calcd for C₂₆H₃₄N₆O₅S, 543.23; found, 543.3. ¹H NMR (400 MHz, DMSO-d₆) δ 9.89 (s, 1H), 8.19 (s, 1H), 7.54-7.39 (m, 2H), 7.29 (d, J=3.8 Hz, 1H), 7.19-7.09 (m, 2H), 6.87 (q, J=4.9 Hz, 1H), 6.65 (d, J=3.7 Hz, 1H), 6.14 (s, 2H), 4.67 (s, 1H), 3.61 (s, 2H), 3.17 (s, 3H), 2.95 (d, J=6.2 Hz, 2H), 2.59 (d, J=5.0 Hz, 3H), 2.03 (s, 5H), 1.84 (s, 1H), 1.70 (q, J=6.7 Hz, 4H), 1.37-1.27 (m, 2H).

### Example 94

### (4-(Methyl ((trans)-4-((N-methylsulfamoyl) methyl) cyclohexyl) amino)-7H-pyrrolo [2, 3-d] pyrimidin-7-yl) methyl 2-(1, 8-diethyl-1, 3, 4, 9-tetrahydropyran [3, 4-b] indol-1-yl) acetate

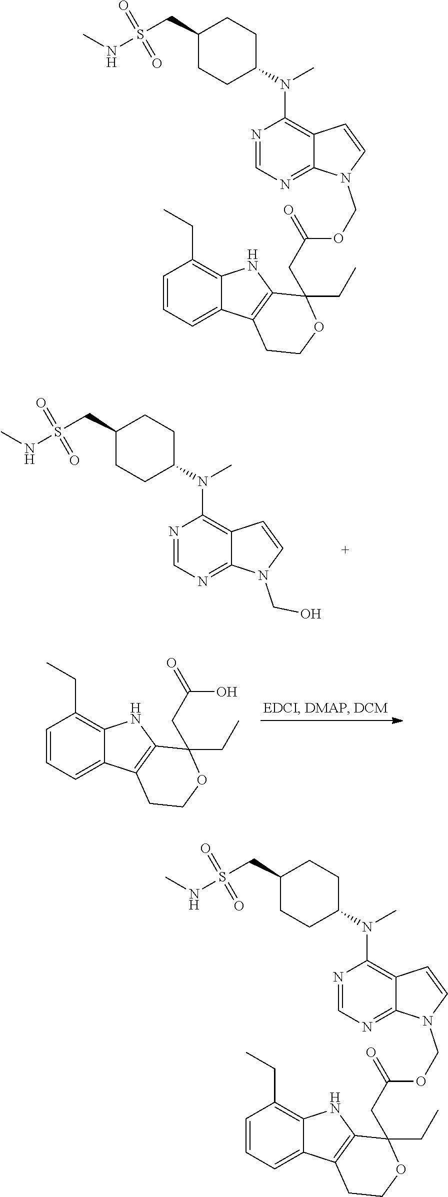 [SMILES: CCc1cccc2c3c([nH]c12)C(CC)(CC(=O)OCn1ccc2c(N(C)[C@H]4CC[C@H](CS(=O)(=O)NC)CC4)ncnc21)OCC3 ; CNS(=O)(=O)C[C@H]1CC[C@H](N(C)c2ncnc3c2ccn3CO)CC1 ; CCc1cccc2c3c([nH]c12)C(CC)(CC(=O)O)OCC3 ; CCc1cccc2c3c([nH]c12)C(CC)(CC(=O)OCn1ccc2c(N(C)[C@H]4CC[C@H](CS(=O)(=O)NC)CC4)ncnc21)OCC3]

### Synthesis of (4-(methyl ((trans)-4-((N-methylsulfamoyl) methyl) cyclohexyl) amino)-7H-pyrrolo [2, 3-d] pyrimidin-7-yl) methyl 2-(1, 8-diethyl-1, 3, 4, 9-tetrahydropyran [3, 4-b] indol-1-yl) acetate

1-((Trans)-4-((7-(hydroxymethyl)-7H-pyrrolo [2, 3-d] pyrimidin-4-yl) (methyl) amino) cyclohexyl)-N-methyl methanesulfonamide (220 mg, 0.6 mmol), 4-dimethylamino pyridine (DMAP, 7 mg, 0.06 mmol), 21, 8-diethyl-1, 3, 4, 9-tetrahydropyran [3, 4-b] indole-1-acetic acid (etodolac, 224 mg, 0.78 mmol), and 1-(3-dimethylaminopropyl)-3-ethylcarbodiimide hydrochloride (EDCI, 173 mg, 0.9 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 0:1) to give the title compound as a white solid, 0.14 g, 36.6% yield. MS (m/z): [M+H]⁺ calcd for C₃₃H₄₄N₆O₅S, 637.31; found, 637.3. ¹H NMR (400 MHz, DMSO-d₆) δ 10.42 (s, 1H), 8.18 (s, 1H), 7.20 (dd, J=7.6, 1.4 Hz, 1H), 7.13 (d, J=3.8 Hz, 1H), 6.94-6.84 (m, 3H), 6.56 (d, J=3.8 Hz, 1H), 6.13-6.02 (m, 2H), 4.66 (s, 1H), 3.92-3.77 (m, 2H), 3.17 (s, 3H), 3.06-2.92 (m, 3H), 2.88-2.76 (m, 3H), 2.69-2.56 (m, 5H), 2.10-2.00 (m, 3H), 1.93-1.81 (m, 2H), 1.76-1.64 (m, 4H), 1.39-1.27 (m, 2H), 1.26-1.18 (m, 3H), 0.59 (t, J=7.3 Hz, 3H).

### Example 95

### 2-(1-(ethylsulfonyl)-3-(4-(7-(2-(4-isobutylphenyl) propanoyl)-7H-pyrrolo [2, 3-d] pyrimidin-4-yl)-1H-pyrazol-1-yl) azetidin-3-yl) acetonitrile

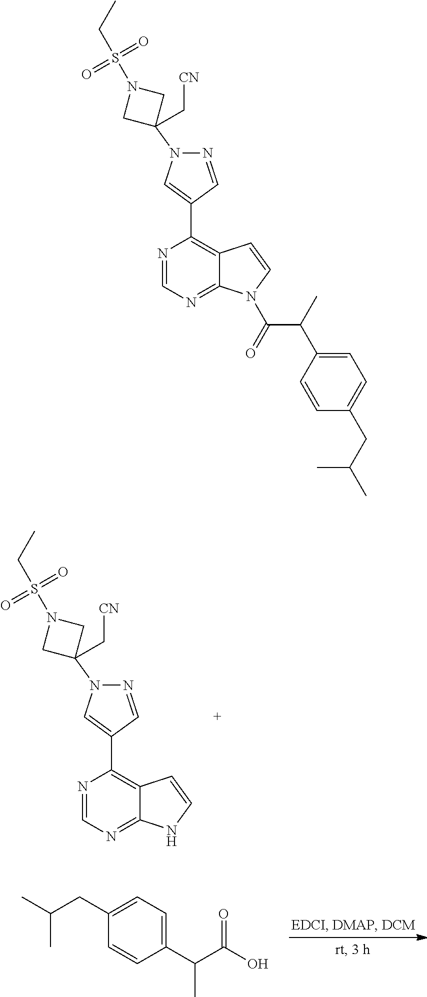

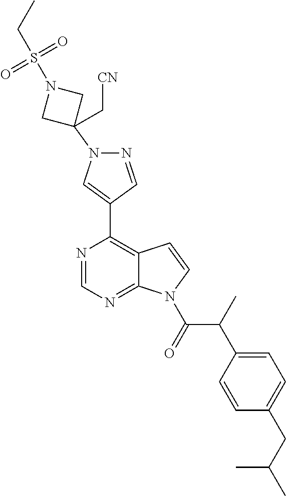 [SMILES: CCS(=O)(=O)N1CC(CC#N)(n2cc(-c3ncnc4c3ccn4C(=O)C(C)c3ccc(CC(C)C)cc3)cn2)C1]

### Synthesis of 2-(1-(ethylsulfonyl)-3-(4-(7-(2-(4-isobutylphenyl) propanoyl)-7H-pyrrolo [2, 3-d] pyrimidin-4-yl)-1H-pyrazol-1-yl) azetidin-3-yl) acetonitrile

1-(Ethylsulfonyl)-3-[4-(7H-pyrrolo [2, 3-d] pyrimidin-4-yl)-1H-pyrazol-1-yl]-3-azetidine acetonitrile (baricitinib, 1113 mg, 3 mmol), 4-dimethylamino pyridine (DMAP, 36.6 mg, 0.3 mmol), 2-(4-isobutylphenyl) propanoic acid (ibuprofen, 742 mg, 3.6 mmol) and 1-(3-dimethylaminopropyl)-3-ethylcarbodiimide hydrochloride (EDCI, 864 mg, 4.5 mmol) were dissolved in dichloromethane (30 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=2:1 to 1:2) to give the title compound as a white solid, 0.12 g, 71.5% yield. MS (m/z): [M+H]⁺ calcd for C₂₉H₃₃N₇O₃S, 560.69; found, 560.3. ¹H NMR (400 MHz, DMSO-d₆) δ 8.99 (d, J=4.2 Hz, 2H), 8.50 (s, 1H), 8.14 (d, J=4.2 Hz, 1H), 7.40 (d, J=4.2 Hz, 1H), 7.37-7.29 (m, 2H), 7.08 (d, J=7.9 Hz, 2H), 5.99 (q, J=6.9 Hz, 1H), 4.59 (d, J=9.1 Hz, 2H), 4.24 (d, J=9.1 Hz, 2H), 3.68 (s, 2H), 3.23 (q, J=7.3 Hz, 2H), 2.35 (d, J=7.2 Hz, 2H), 1.76 (hept, J=6.7 Hz, 1H), 1.58 (d, J=6.9 Hz, 3H), 1.24 (t, J=7.3 Hz, 3H), 0.80 (d, J=6.6 Hz, 6H).

### Example 96

### 1-((Trans)-4-((7-(2-(4-isobutylphenyl) propanoyl)-7H-pyrrolo [2, 3-d] pyrimidin-4-yl) (methyl) amino) cyclohexyl)-N-methyl methanesulfonamide

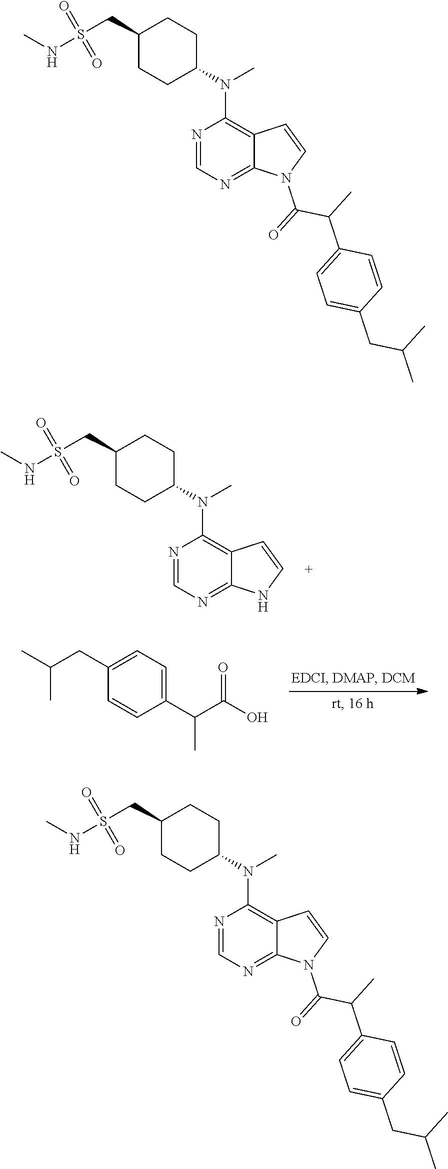

### Synthesis of 1-((trans)-4-((7-(2-(4-isobutylphenyl) propanoyl)-7H-pyrrolo [2, 3-d] pyrimidin-4-yl) (methyl) amino) cyclohexyl)-N-methyl methanesulfonamide

N-methyl-1-((trans)-4-(methyl (7H-pyrrolo [2, 3-d] pyrimidin-4-yl) amino) cyclohexyl) methanesulfonamide (oclacitinib, 150 mg, 0.44 mmol), 4-dimethylamino pyridine (DMAP, 6 mg, 0.044 mmol), 2-(4-isobutylphenyl) propanoic acid (ibuprofen, 96 mg, 0.468 mmol) and 1-(3-dimethylaminopropyl)-3-ethylcarbodiimide hydrochloride (EDCI, 128 mg, 0.666 mmol) were dissolved in dichloromethane (6 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=4:1 to 2:1) to give the title compound as a white solid, 0.07 g, 30% yield. MS (m/z): [M+H]⁺ calcd for C₂₈H₃₉N₅O₃S, 526.28; found, 526.1. ¹H NMR (400 MHz, DMSO-d₆) δ 8.36 (s, 1H), 7.66 (d, J=4.2 Hz, 1H), 7.33-7.26 (m, 2H), 7.07 (d, J=8.0 Hz, 2H), 6.95-6.81 (m, 2H), 6.10 (q, J=6.9 Hz, 1H), 4.66 (s, 1H), 3.15 (s, 3H), 2.94 (d, J=6.2 Hz, 2H), 2.58 (d, J=5.0 Hz, 3H), 2.36 (d, J=7.1 Hz, 2H), 2.10-1.96 (m, 2H), 1.91-1.60 (m, 6H), 1.52 (d, J=7.0 Hz, 3H), 1.35-1.22 (m, 2H), 0.82 (d, J=6.7 Hz, 6H).

### Example 97

### 4-((4-(((3R, 4R)-1-(2-cyanoacetyl)-4-methylpiperidin-3-yl) (methyl) amino)-7H-pyrrolo [2, 3-d] pyrimidin-7-yl) methoxy)-2-methyl-N-(pyridin-2-yl)-2H-benzo [e] [1, 2] thiazine-3-carboxamide 1, 1-dioxide

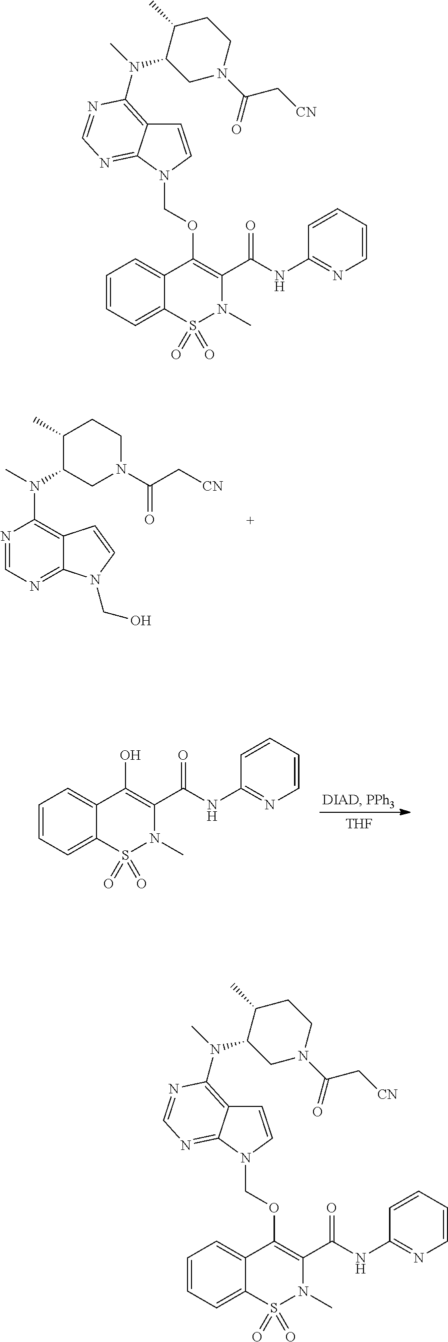 [SMILES: C[C@@H]1CCN(C(=O)CC#N)C[C@@H]1N(C)c1ncnc2c1ccn2COC1=C(C(=O)Nc2ccccn2)N(C)S(=O)(=O)c2ccccc21 ; C[C@@H]1CCN(C(=O)CC#N)C[C@@H]1N(C)c1ncnc2c1ccn2CO ; CN1C(C(=O)Nc2ccccn2)=C(O)c2ccccc2S1(=O)=O ; C[C@@H]1CCN(C(=O)CC#N)C[C@@H]1N(C)c1ncnc2c1ccn2COC1=C(C(=O)Nc2ccccn2)N(C)S(=O)(=O)c2ccccc21 ; "THF"]

### Synthesis of 4-((4-(((3R, 4R)-1-(2-cyanoacetyl)-4-methylpiperidin-3-yl) (methyl) amino)-7H-pyrrolo [2, 3-d] pyrimidin-7-yl) methoxy)-2-methyl-N-(pyridin-2-yl)-2H-benzo [e] [1, 2] thiazine-3-carboxamide 1, 1-dioxide

4-Hydroxy-2-methyl-N-(pyridin-2-yl)-2H-benzo [e] [1, 2] thiazine-3-carboxamide 1, 1-dioxide (piroxicam, 66 mg, 0.2 mmol) and triphenylphosphine (PPh₃, 79 mg, 0.3 mmol) were added to tetrahydrofuran (0.4 mL) and stirred under nitrogen. After cooling to −10° C., diisopropyl azodicarboxylate (DIAD, 53 mg, 0.26 mmol) was added dropwise to the mixture with stirring. After stirring at −10° C. for 20 minutes, the mixture was allowed to warm to room temperature naturally. 3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile (68 mg, 0.2 mmol) was added and stirring was continued. The reaction was monitored by TLC. After complete exhaust of the starting material (1 h), the solvent was evaporated under reduced pressure to give the crude product. The crude product was further separated by silica gel column chromatography (dichloromethane/acetonitrile=20:1 to 9:1) to give the title compound as a yellow solid, 0.005 g, 3.8% yield. MS (m/z): [M+H]⁺ calcd for C₃₂H₃₃N₉O₅S, 656.23, found 656.2.1H NMR (400 MHz, Chloroform-d) δ 15.22 (d, J=17.7 Hz, 1H), 8.52 (t, J=8.4 Hz, 1H), 8.32 (d, J=9.2 Hz, 1H), 8.23 (d, J=3.1 Hz, 1H), 8.07-7.91 (m, 2H), 7.83 (d, J=7.5 Hz, 1H), 7.71-7.55 (m, 3H), 6.70-6.58 (m, 2H), 6.51 (dd, J=9.8, 3.8 Hz, 1H), 4.95 (s, 1H), 4.06 (dd, J=13.0, 4.4 Hz, 1H), 3.85-3.67 (m, 1H), 3.66-3.55 (m, 1H), 3.54-3.36 (m, 4H), 3.27 (d, J=18.4 Hz, 3H), 3.03 (s, 3H), 2.53-2.39 (m, 1H), 1.95-1.85 (m, 1H), 1.73-1.61 (m, 1H), 1.01 (t, J=7.7 Hz, 3H).

### Example 98

### (R)-3-cyclopentyl-3-(4-(7-2-(4-isobutylphenyl) propanoyl)-7H-pyrrolo [2, 3-d] pyrimidin-4-yl)-1H-pyrazol-1-yl) propanenitrile

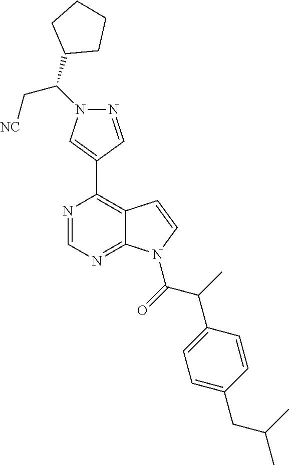 [SMILES: CC(C)Cc1ccc(C(C)C(=O)n2ccc3c(-c4cnn([C@H](CC#N)C5CCCC5)c4)ncnc32)cc1]

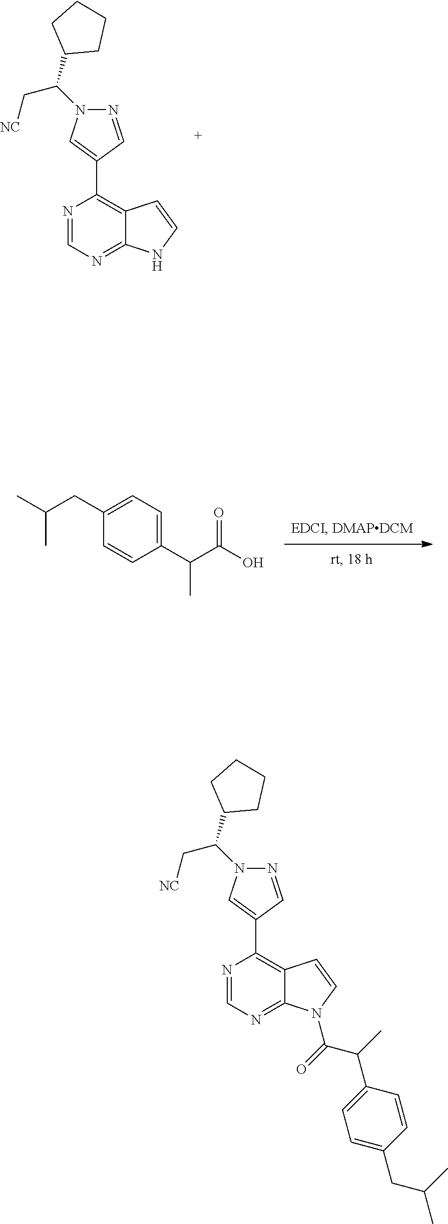 [SMILES: N#CC[C@H](C1CCCC1)n1cc(-c2ncnc3[nH]ccc23)cn1 ; CC(C)Cc1ccc(C(C)C(=O)O)cc1 ; CC(C)Cc1ccc(C(C)C(=O)n2ccc3c(-c4cnn([C@H](CC#N)C5CCCC5)c4)ncnc32)cc1]

### Synthesis of (R)-3-cyclopentyl-3-(4-(7-2-(4-isobutylphenyl) propanoyl)-7H-pyrrolo [2, 3-d] pyrimidin-4-yl)-1H-pyrazol-1-yl) propanenitrile

(R)-3-(4-(7h-pyrrolo [2, 3-d] pyridin-4-yl)-1h-pyrazol-1-yl)-3-cyclopentylpropane (Ruxolitinib, 50 mg, 0.163 mmol), 4-dimethylamino pyridine (DMAP, 2 mg, 0.016 mmol), 2-(4-isobutylphenyl) propanoic acid (ibuprofen, 35.5 mg, 0.172 mmol), and 1-(3-dimethylaminopropyl)-3-ethylcarbodiimide hydrochloride (EDCI, 47 mg, 0.245 mmol) were dissolved in dichloromethane (2 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (petroleum ether/ethyl acetate=4:1 to 3:1) to give the title compound as a white solid, 0.05 g, 61% yield. MS (m/z): [M+H]⁺ calcd for C₃₀H₃₄N₆O₄, 495.64; found, 495.2. ¹H NMR (400 MHz, DMSO-d₆) δ 8.97 (s, 1H), 8.88 (d, J=1.6 Hz, 1H), 8.39 (d, J=11.3 Hz, 1H), 8.12 (d, J=4.2 Hz, 1H), 7.40-7.28 (m, 3H), 7.09 (dd, J=7.9, 5.8 Hz, 2H), 6.00 (q, J=6.9 Hz, 1H), 4.54 (td, J=9.6, 4.6 Hz, 1H), 3.28-3.15 (m, 2H), 2.47-2.31 (m, 3H), 1.88-1.71 (m, 2H), 1.65-1.40 (m, 5H), 1.40-1.16 (m, 5H), 0.99-0.76 (m, 6H).

### Example 99

### (4-(1-((R)-2-cyano-1-cyclopentylethyl)-1H-pyrazol-4-yl)-7H-pyrrolo [2, 3-d] pyrimidin-7-yl) methyl 2-(3-benzoylphenyl) propanoate

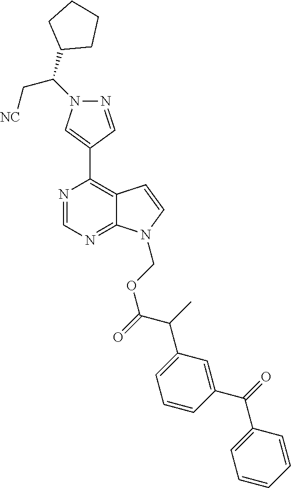 [SMILES: CC(C(=O)OCn1ccc2c(-c3cnn([C@H](CC#N)C4CCCC4)c3)ncnc21)c1cccc(C(=O)c2ccccc2)c1]

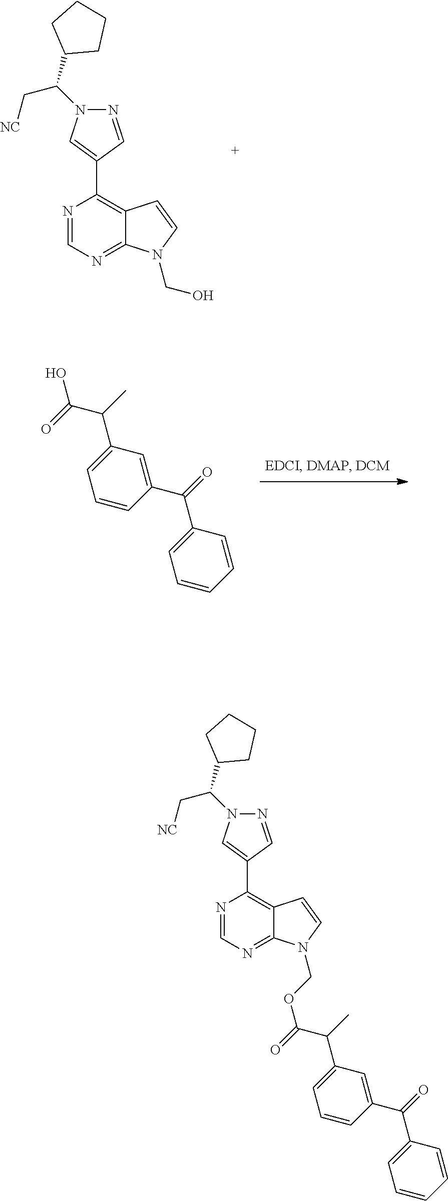 [SMILES: N#CC[C@H](C1CCCC1)n1cc(-c2ncnc3c2ccn3CO)cn1 ; CC(C(=O)O)c1cccc(C(=O)c2ccccc2)c1 ; CC(C(=O)OCn1ccc2c(-c3cnn([C@H](CC#N)C4CCCC4)c3)ncnc21)c1cccc(C(=O)c2ccccc2)c1]

### Synthesis of (4-(1-((R)-2-cyano-1-cyclopentylethyl)-1H-pyrazol-4-yl)-7H-pyrrolo [2, 3-d] pyrimidin-7-yl) methyl 2-(3-benzoylphenyl) propanoate

(R)-3-cyclopentyl-3-(4-(7-(hydroxymethyl)-7H-pyrrolo [2, 3-d] pyrimidin-4-yl)-1H-pyrazol-1-yl) propanenitrile (170 mg, 0.5 mmol), 4-dimethylamino pyridine (DMAP, 61 mg, 0.5 mmol), 2-(3-benzoylphenyl) propanoic acid (ketoprofen, 191 mg, 1.5 mmol) and 1-(3-dimethylaminopropyl)-3-ethylcarbodiimide hydrochloride (EDCI, 144 mg, 0.75 mmol) was dissolved in a mixed solvent of dichloromethane (8.5 mL) and dimethylformamide (0.3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.143 g, 50% yield. MS (m/z): [M+H]⁺ calcd for C₃₄H₃₂N₆O₃, 573.25; found, 573.3. ¹H NMR (400 MHz, DMSO-d₆) δ 8.83 (d, J=0.7 Hz, 1H), 8.72 (s, 1H), 8.38 (s, 1H), 7.69 (d, J=3.8 Hz, 1H), 7.68-7.62 (m, 3H), 7.58 (d, J=1.8 Hz, 1H), 7.57-7.49 (m, 4H), 7.44 (t, J=7.6 Hz, 1H), 7.08 (d, J=3.8 Hz, 1H), 6.33-6.22 (m, 2H), 4.55 (td, J=9.7, 4.2 Hz, 1H), 3.99 (q, J=7.0 Hz, 1H), 3.32-3.15 (m, 2H), 2.48-2.35 (m, 1H), 1.87-1.76 (m, 1H), 1.67-1.44 (m, 3H), 1.42-1.37 (m, 3H), 1.36-1.13 (m, 4H).

### Example 100

### (4-(1-((R)-2-cyano-1-cyclopentylethyl)-1H-pyrazol-4-yl)-7H-pyrrolo [2, 3-d] pyrimidin-7-yl) methyl 2-(2-fluoro-[1, 1′-biphenyl]-4-yl) propanoate

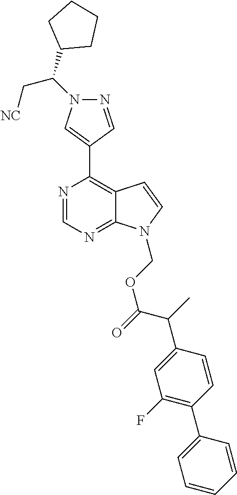 [SMILES: CC(C(=O)OCn1ccc2c(-c3cnn([C@H](CC#N)C4CCCC4)c3)ncnc21)c1ccc(-c2ccccc2)c(F)c1]

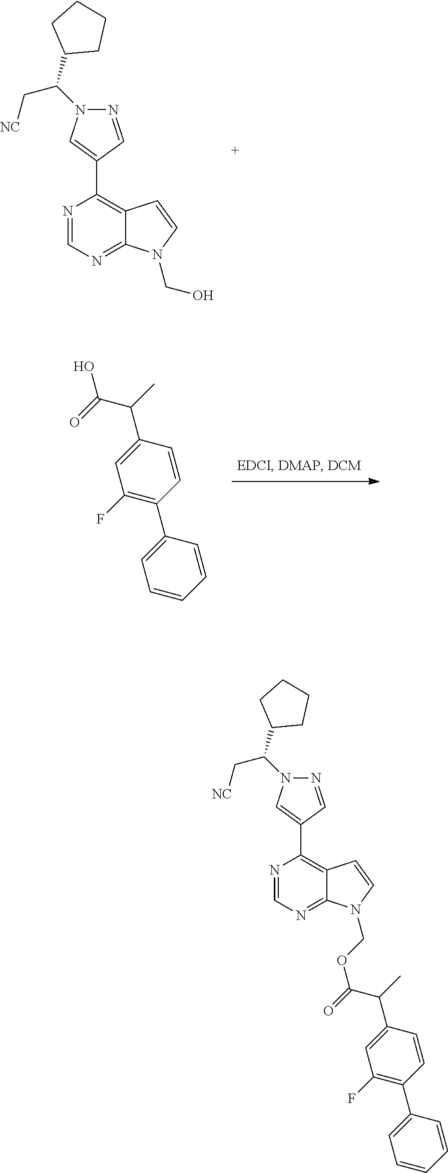 [SMILES: N#CC[C@H](C1CCCC1)n1cc(-c2ncnc3c2ccn3CO)cn1 ; CC(C(=O)O)c1ccc(-c2ccccc2)c(F)c1 ; CC(C(=O)OCn1ccc2c(-c3cnn([C@H](CC#N)C4CCCC4)c3)ncnc21)c1ccc(-c2ccccc2)c(F)c1]

### Synthesis of (4-(1-((R)-2-cyano-1-cyclopentylethyl)-1H-pyrazol-4-yl)-7H-pyrrolo [2, 3-d] pyrimidin-7-yl) methyl 2-(2-fluoro-[1, 1′-biphenyl]-4-yl) propanoate

(R)-3-cyclopentyl-3-(4-(7-(hydroxymethyl)-7H-pyrrolo [2, 3-d] pyrimidin-4-yl)-1H-pyrazol-1-yl) propanenitrile (170 mg, 0.5 mmol), 4-dimethylamino pyridine (DMAP, 61 mg, 0.5 mmol), 2-(2-fluoro-4-biphenyl) propanoic acid (flurbiprofen, 183 mg, 0.75 mmol), and 1-(3-dimethylaminopropyl)-3-ethylcarbodiimide hydrochloride (EDCI, 144 mg, 0.75 mmol) were dissolved in dichloromethane (8.5 mL) and dimethylformamide (0.3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.15 g, 53.3% yield. MS (m/z): [M+H]⁺ calcd for C₃₃H₃₁FN₆O₂, 563.25; found, 563.3. H NMR (400 MHz, DMSO-d₆) δ 8.83 (s, 1H), 8.75 (s, 1H), 8.39 (s, 1H), 7.73 (d, J=3.7 Hz, 1H), 7.50-7.31 (m, 6H), 7.17-7.07 (m, 3H), 6.35-6.24 (m, 2H), 4.54 (td, J=9.7, 4.2 Hz, 1H), 3.92 (q, J=7.1 Hz, 1H), 3.33-3.14 (m, 2H), 2.42 (h, J=8.5 Hz, 1H), 1.82 (dtd, J=12.0, 7.6, 4.5 Hz, 1H), 1.64-1.48 (m, 3H), 1.48-1.11 (m, 7H).

### Example 101

### (R)-(4-(1-(2-cyano-1-cyclopentylethyl)-1H-pyrazol-4-yl)-7H-pyrrolo [2, 3-d] pyrimidin-7-yl) methyl 2-(2-((2, 6-dichlorophenyl) amino) phenyl) acetate

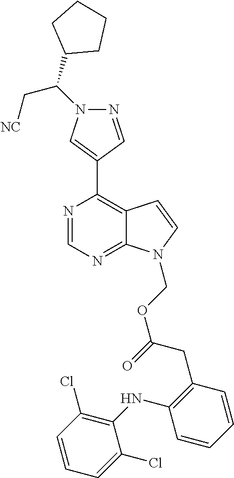 [SMILES: N#CC[C@H](C1CCCC1)n1cc(-c2ncnc3c2ccn3COC(=O)Cc2ccccc2Nc2c(Cl)cccc2Cl)cn1]

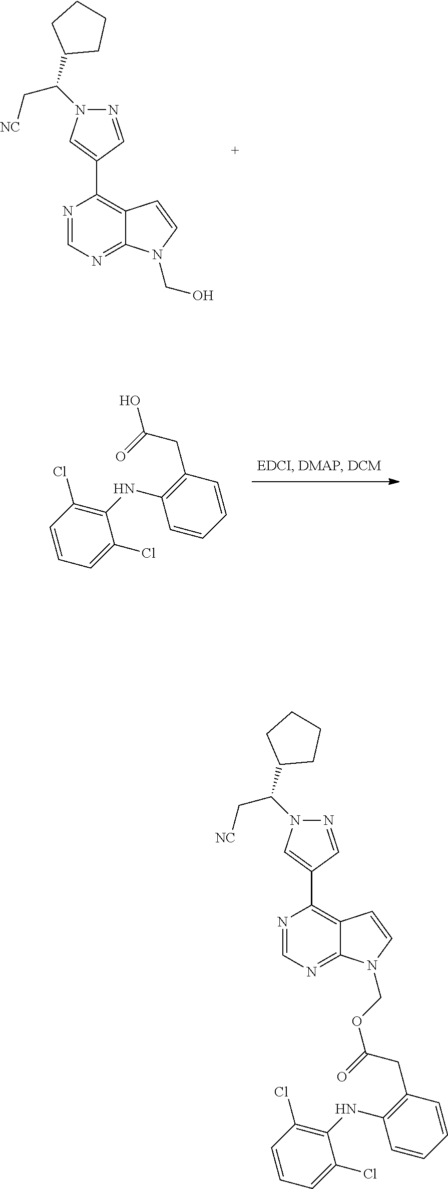 [SMILES: N#CC[C@H](C1CCCC1)n1cc(-c2ncnc3c2ccn3CO)cn1 ; O=C(O)Cc1ccccc1Nc1c(Cl)cccc1Cl ; N#CC[C@H](C1CCCC1)n1cc(-c2ncnc3c2ccn3COC(=O)Cc2ccccc2Nc2c(Cl)cccc2Cl)cn1]

### Synthesis of (R)-(4-(1-(2-cyano-1-cyclopentylethyl)-1H-pyrazol-4-yl)-7H-pyrrolo [2, 3-d] pyrimidin-7-yl) methyl 2-(2-((2, 6-dichlorophenyl) amino) phenyl) acetate

(R)-3-cyclopentyl-3-(4-(7-(hydroxymethyl)-7H-pyrrolo [2, 3-d] pyrimidin-4-yl)-1H-pyrazol-1-yl) propanenitrile (170 mg, 0.5 mmol), 4-dimethylamino pyridine (DMAP, 61 mg, 0.5 mmol), 2-(2, 6-dichloroanilino) phenylacetic acid (diclofenac, 222 mg, 0.75 mmol), and 1-(3-dimethylaminopropyl)-3-ethylcarbodiimide hydrochloride (EDCI, 144 mg, 0.75 mmol) was dissolved in dichloromethane (8.5 mL) and dimethylformamide (0.3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.12 g, 39.1% yield. MS (m/z): [M+H]⁺ calcd for C₃₂H₂₉Cl₂N₇O₂, 614.18; found, 614.2. ¹H NMR (400 MHz, DMSO-d₀) δ 8.86-8.73 (m, 2H), 8.40 (s, 1H), 7.81-7.68 (m, 2H), 7.49 (d, J=8.1 Hz, 2H), 7.26-6.94 (m, 4H), 6.81 (td, J=7.4, 1.1 Hz, 1H), 6.31 (s, 2H), 6.21 (d, J=8.1 Hz, 1H), 4.55 (td, J=9.7, 4.2 Hz, 1H), 3.87 (d, J=14.3 Hz, 2H), 3.24 (qd, J=17.2, 6.9 Hz, 2H), 2.43 (h, J=8.3 Hz, 1H), 1.82 (qt, J=7.5, 5.4, 4.1 Hz, 1H), 1.67-1.40 (m, 4H), 1.40-1.13 (m, 3H).

### Example 102

### (4-(1-((R)-2-cyano-1-cyclopentylethyl)-1H-pyrazol-4-yl)-7H-pyrrolo [2, 3-d] pyrimidin-7-yl) methyl 2-(3-phenoxyphenyl) propanoate

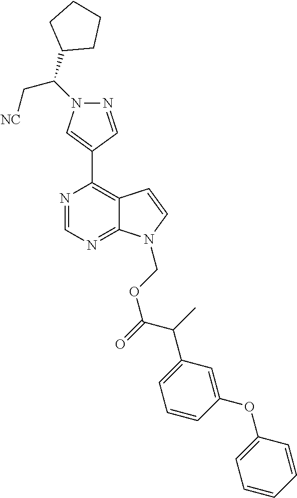 [SMILES: CC(C(=O)OCn1ccc2c(-c3cnn([C@H](CC#N)C4CCCC4)c3)ncnc21)c1cccc(Oc2ccccc2)c1]

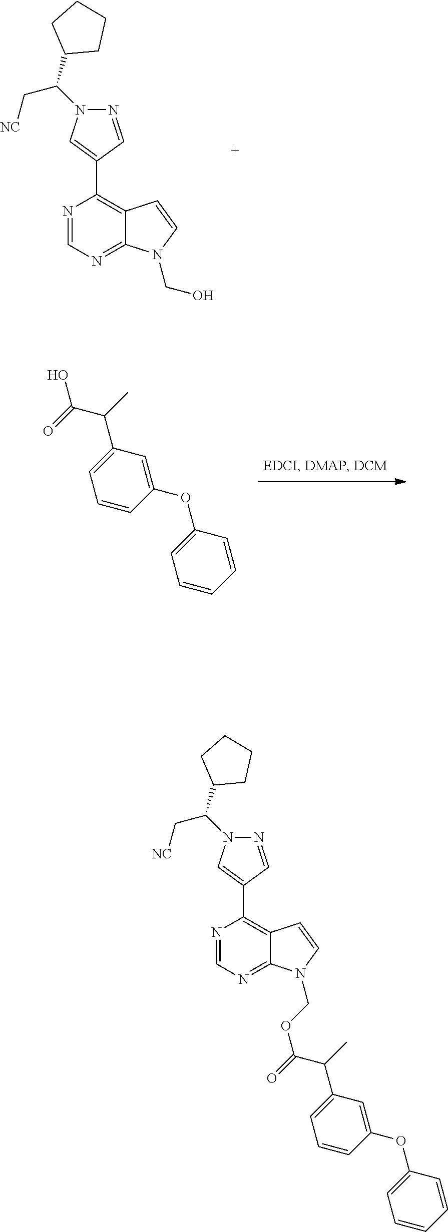 [SMILES: N#CC[C@H](C1CCCC1)n1cc(-c2ncnc3c2ccn3CO)cn1 ; CC(C(=O)O)c1cccc(Oc2ccccc2)c1 ; CC(C(=O)OCn1ccc2c(-c3cnn([C@H](CC#N)C4CCCC4)c3)ncnc21)c1cccc(Oc2ccccc2)c1]

### Synthesis of (4-(1-((R)-2-cyano-1-cyclopentylethyl)-1H-pyrazol-4-yl)-7H-pyrrolo [2, 3-d] pyrimidin-7-yl) methyl 2-(3-phenoxyphenyl) propanoate

(R)-3-cyclopentyl-3-(4-(7-(hydroxymethyl)-7H-pyrrolo [2, 3-d] pyrimidin-4-yl)-1H-pyrazol-1-yl) propanenitrile (170 mg, 0.5 mmol), 4-dimethylamino pyridine (DMAP, 61 mg, 0.5 mmol), 2-(3-phenoxyphenyl) propanoic acid (fenoprofen, 182 mg, 0.75 mmol), and 1-(3-dimethylaminopropyl)-3-ethylcarbodiimide hydrochloride (EDCI, 144 mg, 0.75 mmol) were dissolved in dichloromethane (8.5 mL) and dimethylformamide (0.3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.133 g, 457.5% yield. MS (m/z): [M+H]⁺ calcd for C₃₃H₃₂N₆O₃, 561.25; found, 561.4. H NMR (400 MHz, DMSO-d₆) δ 8.83 (s, 1H), 8.74 (s, 1H), 8.40 (s, 1H), 7.67 (d, J=3.8 Hz, 1H), 7.33 (dd, J=8.5, 7.3 Hz, 2H), 7.24 (t, J=7.9 Hz, 1H), 7.14-7.05 (m, 2H), 6.97 (dt, J=7.7, 1.2 Hz, 1H), 6.93-6.86 (m, 2H), 6.86-6.75 (m, 2H), 6.32-6.20 (m, 2H), 4.55 (td, J=9.7, 4.2 Hz, 1H), 3.83 (q, J=7.0 Hz, 1H), 3.28 (dd, J=17.2, 9.6 Hz, 1H), 3.19 (dd, J=17.2, 4.2 Hz, 1H), 2.42 (h, J=8.6 Hz, 1H), 1.82 (dtd, J=11.9, 7.5, 4.4 Hz, 1H), 1.66-1.43 (m, 4H), 1.43-1.12 (m, 6H).

### Example 103

### (4-(1-((R)-2-cyano-1-cyclopentylethyl)-1H-pyrazol-4-yl)-7H-pyrrolo [2, 3-d] pyrimidin-7-yl) methyl 2-(4-((2-oxocyclopentyl) methyl) phenyl) propanoate

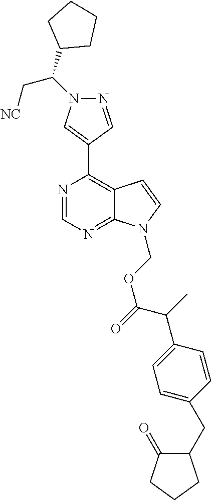 [SMILES: CC(C(=O)OCn1ccc2c(-c3cnn([C@H](CC#N)C4CCCC4)c3)ncnc21)c1ccc(CC2CCCC2=O)cc1]

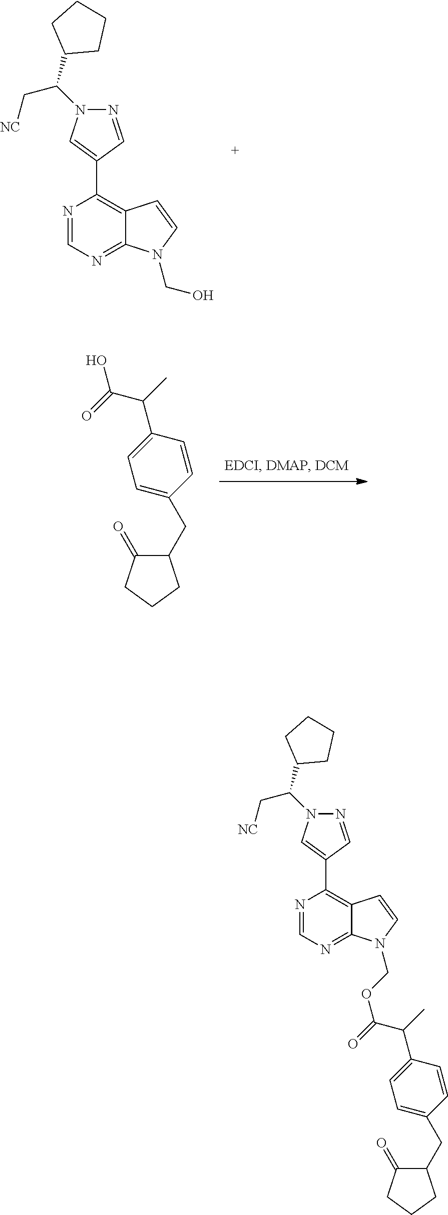 [SMILES: N#CC[C@H](C1CCCC1)n1cc(-c2ncnc3c2ccn3CO)cn1 ; CC(C(=O)O)c1ccc(CC2CCCC2=O)cc1 ; CC(C(=O)OCn1ccc2c(-c3cnn([C@H](CC#N)C4CCCC4)c3)ncnc21)c1ccc(CC2CCCC2=O)cc1]

### Synthesis of (4-(1-((R)-2-cyano-1-cyclopentylethyl)-1H-pyrazol-4-yl)-7H-pyrrolo [2, 3-d] pyrimidin-7-yl) methyl 2-(4-((2-oxocyclopentyl) methyl) phenyl) propanoate

(R)-3-cyclopentyl-3-(4-(7-(hydroxymethyl)-7H-pyrrolo [2, 3-d] pyrimidin-4-yl)-1H-pyrazol-1-yl) propanenitrile (170 mg, 0.5 mmol), 4-dimethylamino pyridine (DMAP, 61 mg, 0.5 mmol), 2-[4-(2-oxocyclopentan-1-ylmethyl) phenyl] propanoic acid (loxoprofen, 185 mg, 0.75 mmol), and 1-(3-dimethylaminopropyl)-3-ethylcarbodiimide hydrochloride (EDCI, 144 mg, 0.75 mmol) were dissolved in dichloromethane (8.5 mL) and dimethylformamide (0.3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.125 g, 44.3% yield. MS (m/z): [M+H]⁺ calcd for C₃₃H₃₆N₆O₃, 565.28; found, 565.3. ¹H NMR (400 MHz, DMSO-d₆) δ 8.82 (s, 1H), 8.74 (d, J=1.7 Hz, 1H), 8.39 (s, 1H), 7.68 (d, J=3.8 Hz, 1H), 7.11-6.96 (m, 5H), 6.28-6.19 (m, 2H), 4.54 (td, J=9.6, 4.2 Hz, 1H), 3.76 (q, J=7.0 Hz, 1H), 3.23 (qd, J=17.1, 6.9 Hz, 2H), 2.92-2.82 (m, 1H), 2.50-2.14 (m, 5H), 2.08-1.89 (m, 1H), 1.86-1.74 (m, 3H), 1.69-1.47 (m, 4H), 1.40-1.12 (m, 7H).

### Example 104

### (R)-2-(4-(1-(2-cyano-1-cyclopentylethyl)-1H-pyrazol-4-yl)-7H-pyrrolo [2, 3-d] pyrimidin-7-yl) methyl 2-(1-(4-chlorobenzoyl)-5-methoxy-2-methyl-1H-indol-3-yl) acetate

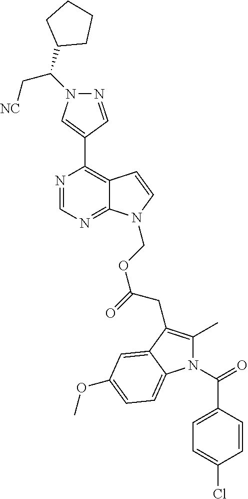 [SMILES: COc1ccc2c(c1)c(CC(=O)OCn1ccc3c(-c4cnn([C@H](CC#N)C5CCCC5)c4)ncnc31)c(C)n2C(=O)c1ccc(Cl)cc1]

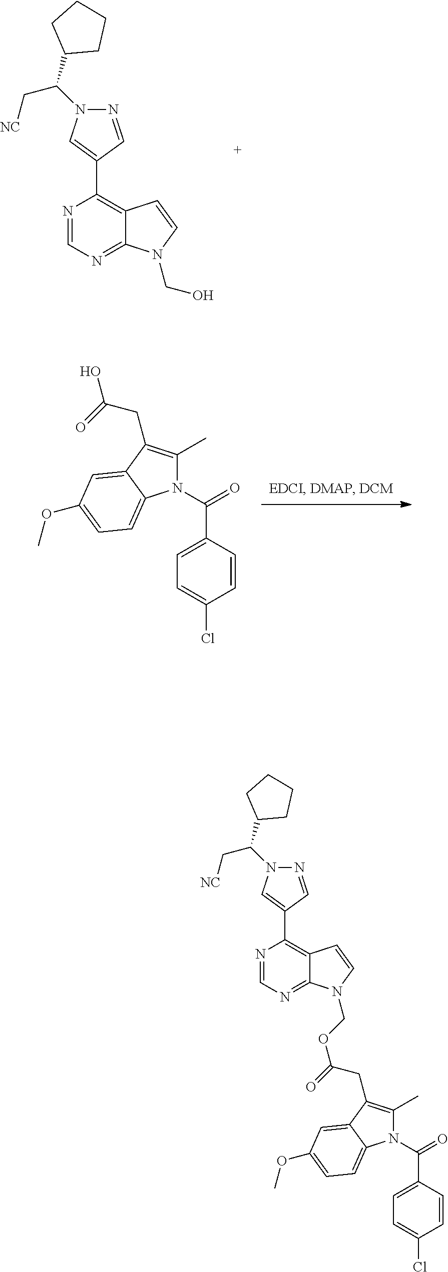 [SMILES: N#CC[C@H](C1CCCC1)n1cc(-c2ncnc3c2ccn3CO)cn1 ; COc1ccc2c(c1)c(CC(=O)O)c(C)n2C(=O)c1ccc(Cl)cc1 ; COc1ccc2c(c1)c(CC(=O)OCn1ccc3c(-c4cnn([C@H](CC#N)C5CCCC5)c4)ncnc31)c(C)n2C(=O)c1ccc(Cl)cc1]

### Synthesis of (R)-2-(4-(1-(2-cyano-1-cyclopentylethyl)-1H-pyrazol-4-yl)-7H-pyrrolo [2, 3-d] pyrimidin-7-yl) methyl 2-(1-(4-chlorobenzoyl)-5-methoxy-2-methyl-1H-indol-3-yl) acetate

(R)-3-cyclopentyl-3-(4-(7-(hydroxymethyl)-7H-pyrrolo [2, 3-d] pyrimidin-4-yl)-1H-pyrazol-1-yl) propanenitrile (170 mg, 0.5 mmol), 4-dimethylamino pyridine (DMAP, 61 mg, 0.5 mmol), 1-(4-chlorobenzoyl)-5-methoxy-2-methyl-1H-indole-3-acetic acid (indomethacin, 277 mg, 0.75 mmol) and 1-(3-dimethylaminopropyl)-3-ethylcarbodiimide hydrochloride (EDCI, 144 mg, 0.75 mmol) were dissolved in dichloromethane (8.5 mL) and dimethylformamide (0.3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a light yellow solid, 0.176 g, 52.1% yield. MS (m/z): [M+H]⁺ calcd for C₃₇H₃₄ClN₇O₄, 676.24; found, 676.3. ¹H NMR (400 MHz, DMSO-d₆) δ 8.84 (s, 1H), 8.75 (s, 1H), 8.40 (s, 1H), 7.71 (d, J=3.8 Hz, 1H), 7.62 (s, 4H), 7.10 (d, J=3.8 Hz, 1H), 6.95-6.85 (m, 2H), 6.66 (dd, J=9.0, 2.5 Hz, 1H), 6.29 (s, 2H), 4.55 (td, J=9.7, 4.2 Hz, 1H), 3.82 (s, 2H), 3.65 (s, 3H), 3.32-3.15 (m, 2H), 2.42 (p, J=8.5 Hz, 1H), 2.13 (s, 3H), 1.89-1.76 (m, 1H), 1.59 (dddd, J=26.4, 12.6, 7.1, 4.5 Hz, 3H), 1.41-1.12 (m, 4H).

### Example 105

### (4-(1-((R)-2-cyano-1-cyclopentylethyl)-1H-pyrazol-4-yl)-7H-pyrrolo [2, 3-d] pyrimidin-7-yl) methyl 2-(4-(1-oxoisoindolin-2-yl) phenyl) butanoate

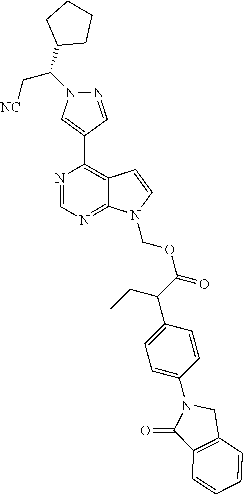 [SMILES: CCC(C(=O)OCn1ccc2c(-c3cnn([C@H](CC#N)C4CCCC4)c3)ncnc21)c1ccc(N2Cc3ccccc3C2=O)cc1]

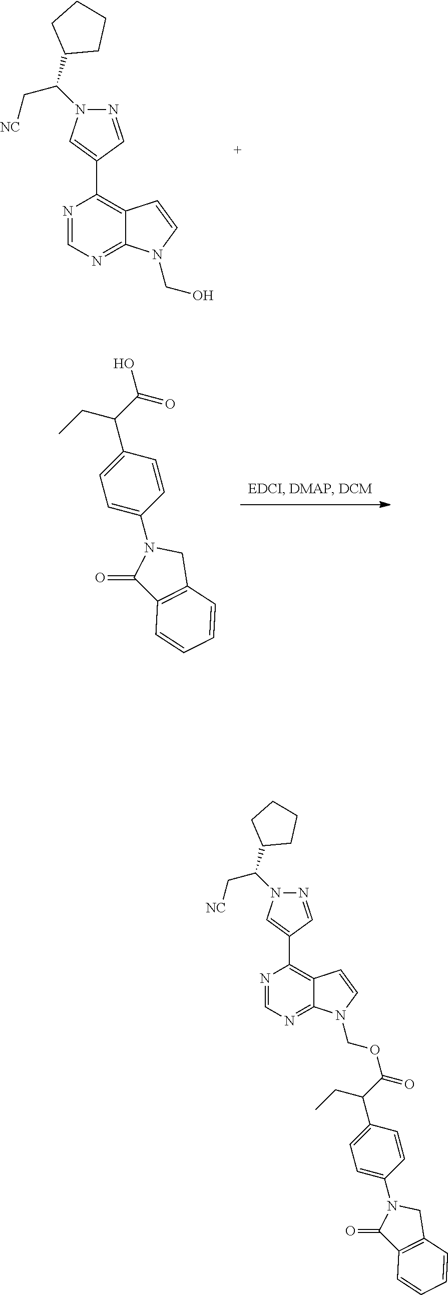 [SMILES: N#CC[C@H](C1CCCC1)n1cc(-c2ncnc3c2ccn3CO)cn1 ; CCC(C(=O)O)c1ccc(N2Cc3ccccc3C2=O)cc1 ; CCC(C(=O)OCn1ccc2c(-c3cnn([C@H](CC#N)C4CCCC4)c3)ncnc21)c1ccc(N2Cc3ccccc3C2=O)cc1]

### Synthesis of (4-(1-((R)-2-cyano-1-cyclopentylethyl)-1H-pyrazol-4-yl)-7H-pyrrolo [2, 3-d] pyrimidin-7-yl) methyl 2-(4-(1-oxoisoindolin-2-yl) phenyl) butanoate

(R)-3-cyclopentyl-3-(4-(7-(hydroxymethyl)-7H-pyrrolo [2, 3-d] pyrimidin-4-yl)-1H-pyrazol-1-yl) propanenitrile (20 mg, 0.058 mmol), 4-dimethylamino pyridine (DMAP, 4 mg, 0.03 mmol), 2-(4-(1-oxoisoindolin-2-yl) phenyl) butanoic acid (indobufen, 26 mg, 0.09 mmol) and 1-(3-dimethylaminopropyl)-3-ethylcarbodiimide hydrochloride (EDCI, 17 mg, 0.09 mmol) were dissolved in a mixed solvent of dichloromethane (0.5 mL) and N, N-dimethylformamide (0.05 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.152 g, 49.6% yield. MS (m/z): [M+H]⁺ calcd for C₃₆H₃₅N₇O₃, 614.28; found, 614.4. ¹H NMR (400 MHz, DMSO-d₆) δ 8.83 (s, 1H), 8.38 (s, 1H), 7.79 (t, J=8.5 Hz, 3H), 7.74-7.67 (m, 2H), 7.67-7.62 (m, 1H), 7.54 (td, J=7.2, 1.5 Hz, 1H), 7.29 (d, J=8.4 Hz, 2H), 7.10 (d, J=3.7 Hz, 1H), 6.35-6.21 (m, 2H), 4.96 (s, 2H), 4.53 (td, J=9.6, 4.2 Hz, 1H), 3.58 (t, J=7.6 Hz, 1H), 3.23 (qd, J=17.2, 6.9 Hz, 2H), 2.42 (h, J=8.4 Hz, 1H), 2.07-1.88 (m, 1H), 1.81 (dtd, J=12.1, 7.6, 4.4 Hz, 1H), 1.76-1.40 (m, 5H), 1.40-1.11 (m, 4H), 0.77 (t, J=7.3 Hz, 3H).

### Example 106

### (R)-(4-(1-(2-cyano-1-cyclopentylethyl)-1H-pyrazol-4-yl)-7H-pyrrolo [2, 3-d] pyrimidin-7-yl) methyl 2-((2, 3-dimethylphenyl) amino) benzoate

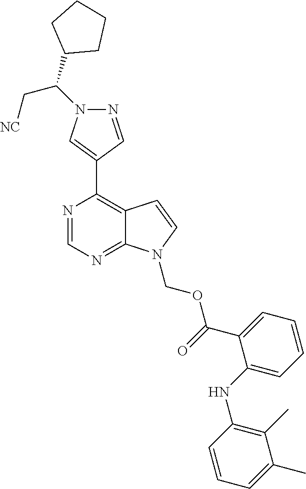 [SMILES: Cc1cccc(Nc2ccccc2C(=O)OCn2ccc3c(-c4cnn([C@H](CC#N)C5CCCC5)c4)ncnc32)c1C]

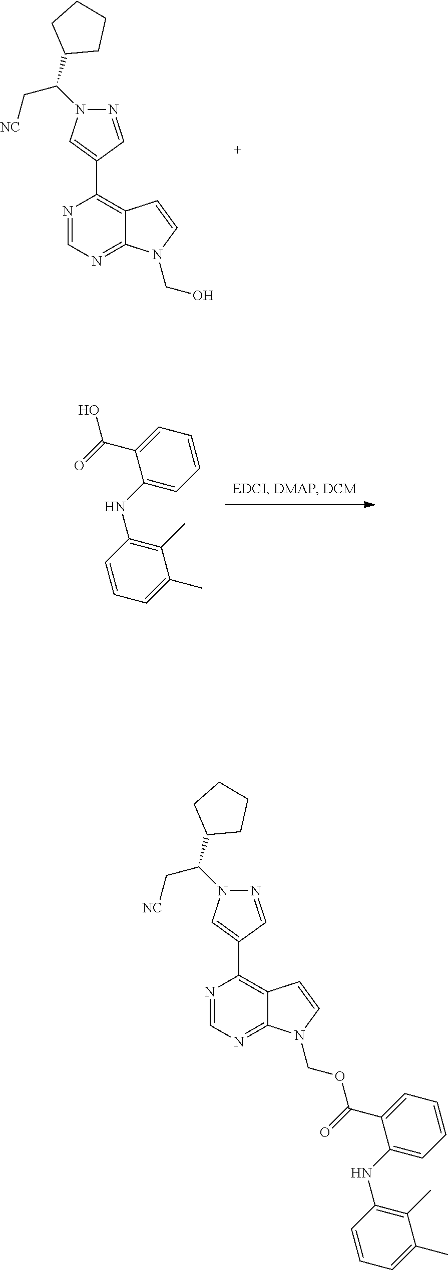 [SMILES: N#CC[C@H](C1CCCC1)n1cc(-c2ncnc3c2ccn3CO)cn1 ; Cc1cccc(Nc2ccccc2C(=O)O)c1C ; Cc1cccc(Nc2ccccc2C(=O)OCn2ccc3c(-c4cnn([C@H](CC#N)C5CCCC5)c4)ncnc32)c1C]

### Synthesis of (R)-(4-(1-(2-cyano-1-cyclopentylethyl)-1H-pyrazol-4-yl)-7H-pyrrolo [2, 3-d] pyrimidin-7-yl) methyl 2-((2, 3-dimethylphenyl) amino) benzoate

(R)-3-cyclopentyl-3-(4-(7-(hydroxymethyl)-7H-pyrrolo [2, 3-d] pyrimidin-4-yl)-1H-pyrazol-1-yl) propanenitrile (171 mg, 0.5 mmol), 4-dimethylaminopyridine (DMAP, 61 mg, 0.5 mmol), 2-((2, 3-dimethylphenyl) amino) benzoic acid (mefenamic acid, 181 mg, 0.75 mmol) and 1-(3-dimethylaminopropyl)-3-ethylcarbodiimide hydrochloride (EDCI, 144 mg, 0.75 mmol) were dissolved in a mixed solvent of dichloromethane (8.5 mL) and N, N-dimethylformamide (0.3 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a yellow solid, 0.147 g, 52.6% yield. MS (m/z): [M+H]⁺ calcd for C₃₃H₃₃N₇O₂, 560.27; found, 560.3. H NMR (400 MHz, DMSO-d₆) δ 9.08 (s, 1H), 8.84 (d, J=18.4 Hz, 2H), 8.42 (s, 1H), 7.91 (d, J=3.8 Hz, 1H), 7.75 (dd, J=8.1, 1.6 Hz, 1H), 7.32 (ddd, J=8.8, 7.2, 1.7 Hz, 1H), 7.20-7.00 (m, 4H), 6.69-6.59 (m, 2H), 6.54 (s, 2H), 4.55 (td, J=9.7, 4.2 Hz, 1H), 3.24 (qd, J=17.2, 6.9 Hz, 2H), 2.43 (q, J=8.4 Hz, 1H), 2.29 (s, 3H), 2.08 (s, 3H), 1.82 (dtd, J=12.0, 7.4, 3.9 Hz, 1H), 1.65-1.50 (m, 3H), 1.49-1.12 (m, 4H).

### Example 107

### (R)-(4-(1-(2-cyano-1-cyclopentylethyl)-1H-pyrazol-4-yl)-7H-pyrrolo [2, 3-d] pyrimidin-7-yl) methyl 2-((3-chloro-2-methylphenyl) amino) benzoate

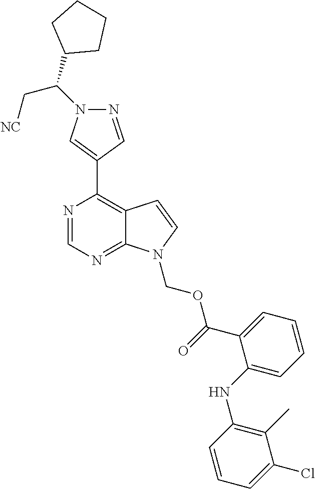 [SMILES: Cc1c(Cl)cccc1Nc1ccccc1C(=O)OCn1ccc2c(-c3cnn([C@H](CC#N)C4CCCC4)c3)ncnc21]

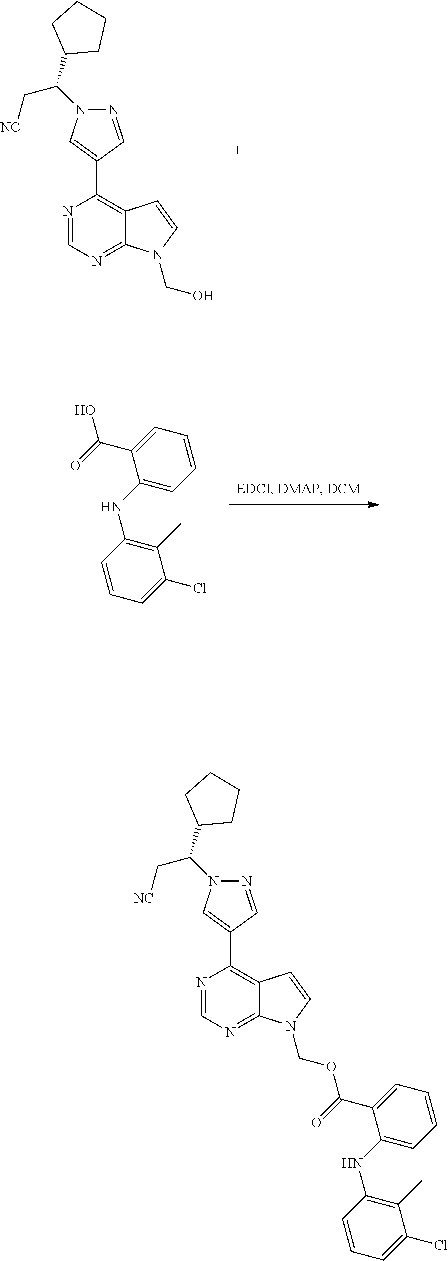 [SMILES: N#CC[C@H](C1CCCC1)n1cc(-c2ncnc3c2ccn3CO)cn1 ; Cc1c(Cl)cccc1Nc1ccccc1C(=O)O ; Cc1c(Cl)cccc1Nc1ccccc1C(=O)OCn1ccc2c(-c3cnn([C@H](CC#N)C4CCCC4)c3)ncnc21]

### Synthesis of (R)-(4-(1-(2-cyano-1-cyclopentylethyl)-1H-pyrazol-4-yl)-7H-pyrrolo [2, 3-d] pyrimidin-7-yl) methyl 2-((3-chloro-2-methylphenyl) amino) benzoate

(R)-3-cyclopentyl-3-(4-(7-(hydroxymethyl)-7H-pyrrolo [2, 3-d] pyrimidin-4-yl)-1H-pyrazol-1-yl) propanenitrile (200 mg, 0.58 mmol), 4-dimethylamino pyridine (DMAP, 35 mg, 0.29 mmol), 2-((3-chloro-2-methylphenyl) amino) benzoic acid (tolfenamic acid, 230 mg, 0.88 mmol) and 1-(3-dimethylaminopropyl)-3-ethylcarbodiimide hydrochloride (EDCI, 168 mg, 0.88 mmol) were dissolved in a mixed solvent of dichloromethane (10 mL) and N, N-dimethylformamide (0.2 0 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a yellow solid, 0.161 g, 55.6% yield. MS (m/z): [M+H]⁺ calcd for C₃₂H₃₀ClN₇O₂, 580.21; found, 580.3.1H NMR (400 MHz, DMSO-d₆) δ 9.13 (s, 1H), 8.83 (d, J=18.6 Hz, 2H), 8.42 (s, 1H), 7.90 (d, J=3.8 Hz, 1H), 7.78 (dd, J=8.0, 1.6 Hz, 1H), 7.37 (ddd, J=8.8, 7.1, 1.7 Hz, 1H), 7.32-7.21 (m, 3H), 7.17 (d, J=3.8 Hz, 1H), 6.78-6.68 (m, 2H), 6.54 (s, 2H), 4.55 (td, J=9.6, 4.2 Hz, 1H), 3.33-3.15 (m, 2H), 2.43 (q, J=8.4 Hz, 1H), 2.24 (s, 3H), 1.82 (dtd, J=11.9, 7.4, 4.2 Hz, 1H), 1.59 (ddd, J=23.3, 7.9, 5.7 Hz, 2H), 1.47-1.12 (m, 5H).

### Example 108

### (4-(1-((R)-2-cyano-1-cyclopentylethyl)-1H-pyrazol-4-yl)-7H-pyrrolo [2, 3-d] pyrimidin-7-yl) methyl 2-(4-isobutylphenyl) propanoate

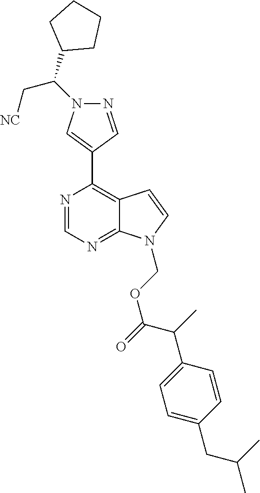 [SMILES: CC(C)Cc1ccc(C(C)C(=O)OCn2ccc3c(-c4cnn([C@H](CC#N)C5CCCC5)c4)ncnc32)cc1]

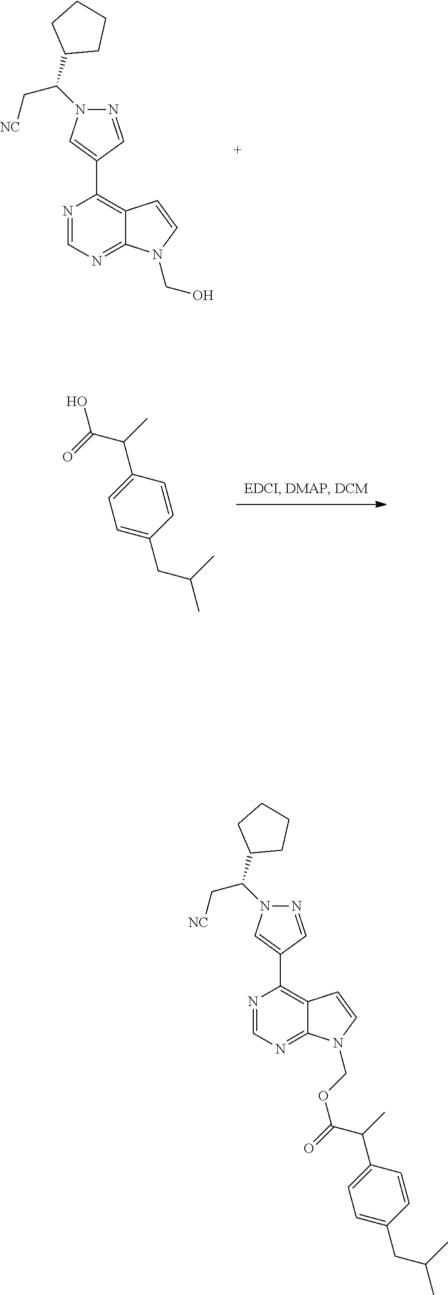 [SMILES: N#CC[C@H](C1CCCC1)n1cc(-c2ncnc3c2ccn3CO)cn1 ; CC(C)Cc1ccc(C(C)C(=O)O)cc1 ; CC(C)Cc1ccc(C(C)C(=O)OCn2ccc3c(-c4cnn([C@H](CC#N)C5CCCC5)c4)ncnc32)cc1]

### Synthesis of (4-(1-((R)-2-cyano-1-cyclopentylethyl)-1H-pyrazol-4-yl)-7H-pyrrolo [2, 3-d] pyrimidin-7-yl) methyl 2-(4-isobutylphenyl) propanoate

(R)-3-cyclopentyl-3-(4-(7-(hydroxymethyl)-7H-pyrrolo [2, 3-d] pyrimidin-4-yl)-1H-pyrazol-1-yl) propanenitrile (201 mg, 0.5 mmol), 4-dimethylamino pyridine (DMAP, 61 mg, 0.5 mmol), 2-(4-isobutylphenyl) propanoic acid (ibuprofen, 123.6 mg, 0.6 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.145 g, 55.3% yield. MS (m/z): [M+H]⁺ calcd for C₃₁H₃₆N₆O₂, 525.29, found 525.3. ¹H NMR (400 MHz, DMSO-d₆) δ 8.83 (s, 1H), 8.74 (s, 1H), 8.39 (s, 1H), 7.68 (d, J=3.8 Hz, 1H), 7.12-7.02 (m, 3H), 6.95 (d, J=7.9 Hz, 2H), 6.25 (q, J=10.7 Hz, 2H), 4.55 (td, J=9.7, 4.2 Hz, 1H), 3.76 (q, J=7.0 Hz, 1H), 3.30-3.15 (m, 2H), 2.43 (q, J=8.5 Hz, 1H), 2.32 (d, J=7.1 Hz, 2H), 1.89-1.77 (m, 1H), 1.70 (hept, J=6.8 Hz, 1H), 1.64-1.48 (m, 3H), 1.41-1.13 (m, 7H), 0.76 (dd, J=6.6, 1.0 Hz, 6H).

### Example 109

### (4-(1-((R)-2-cyano-1-cyclopentylethyl)-1H-pyrazol-4-yl)-7H-pyrrolo [2, 3-d] pyrimidin-7-yl) methyl 2-(1, 8-diethyl-1, 3, 4, 9-tetrahydropyran [3, 4-b] indol-1-yl) acetate

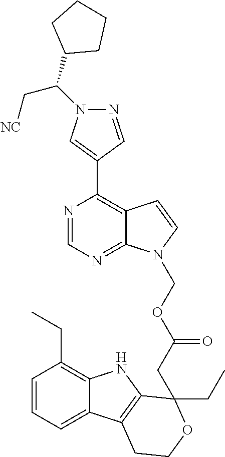 [SMILES: CCc1cccc2c3c([nH]c12)C(CC)(CC(=O)OCn1ccc2c(-c4cnn([C@H](CC#N)C5CCCC5)c4)ncnc21)OCC3]

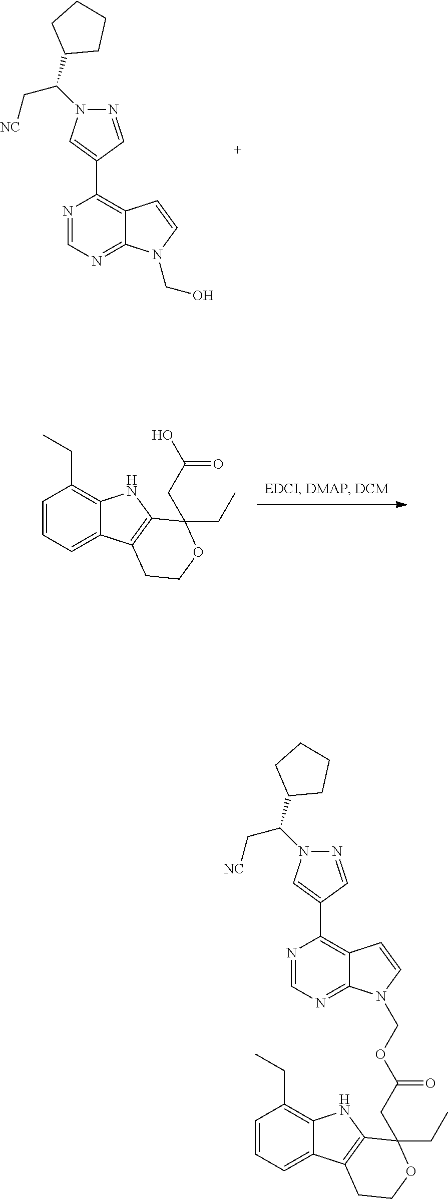 [SMILES: N#CC[C@H](C1CCCC1)n1cc(-c2ncnc3c2ccn3CO)cn1 ; CCc1cccc2c3c([nH]c12)C(CC)(CC(=O)O)OCC3 ; CCc1cccc2c3c([nH]c12)C(CC)(CC(=O)OCn1ccc2c(-c4cnn([C@H](CC#N)C5CCCC5)c4)ncnc21)OCC3]

### Synthesis of (4-(1-((R)-2-cyano-1-cyclopentylethyl)-1H-pyrazol-4-yl)-7H-pyrrolo [2, 3-d] pyrimidin-7-yl) methyl 2-(1, 8-diethyl-1, 3, 4, 9-tetrahydropyran [3, 4-b] indol-1-yl) acetate

(R)-3-cyclopentyl-3-(4-(7-(hydroxymethyl)-7H-pyrrolo [2, 3-d] pyrimidin-4-yl)-1H-pyrazol-1-yl) propanenitrile (171 mg, 0.5 mmol), 4-dimethylamino pyridine (DMAP, 61 mg, 0.5 mmol), 1, 8-diethyl-1, 3, 4, 9-tetrahydropyran [3, 4-b] indole-1-acetic acid (etodolac, 216 mg, 0.75 mmol) and 1-(3-dimethylaminopropyl)-3-ethylcarbodiimide hydrochloride (EDCI, 144 mg, 0.75 mmol) were dissolved in a mixed solvent of dichloromethane (8.5 mL) and dimethylformamide (0.3 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.153 g, 50.5% yield. MS (m/z): [M+H]⁺ calcd for C₃₅H₃₉N₇O₃, 606.31; found, 606.3. ¹H NMR (400 MHz, DMSO-d₆) δ 10.42 (s, 1H), 8.83 (s, 1H), 8.76 (s, 1H), 8.40 (s, 1H), 7.56 (dd, J=3.8, 1.5 Hz, 1H), 7.15 (dd, J=7.3, 1.5 Hz, 1H), 7.01 (d, J=3.7 Hz, 1H), 6.90-6.79 (m, 2H), 6.19 (d, J=1.4 Hz, 2H), 4.56 (td, J=9.7, 4.2 Hz, 1H), 3.82 (td, J=11.1, 5.4 Hz, 2H), 3.31-3.15 (m, 2H), 3.05 (d, J=13.6 Hz, 1H), 2.88-2.69 (m, 3H), 2.60 (ddd, J=15.0, 8.1, 5.3 Hz, 1H), 2.44 (q, J=8.4 Hz, 1H), 2.07-1.76 (m, 3H), 1.68-1.41 (m, 4H), 1.41-1.14 (m, 7H), 0.57 (t, J=7.2 Hz, 3H).

### Example 110

### (R)-(4-(1-(2-cyano-1-cyclopentylethyl)-1H-pyrazol-4-yl)-7H-pyrrolo [2, 3-d] pyrimidin-7-yl) methyl 2-(4-acetamidophenyl) acetate

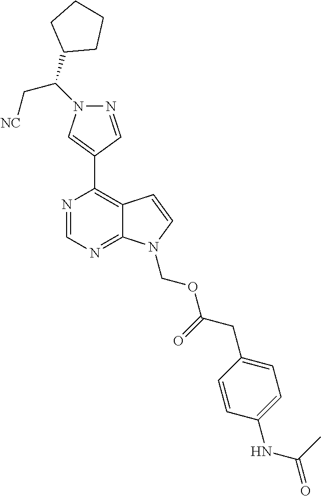 [SMILES: CC(=O)Nc1ccc(CC(=O)OCn2ccc3c(-c4cnn([C@H](CC#N)C5CCCC5)c4)ncnc32)cc1]

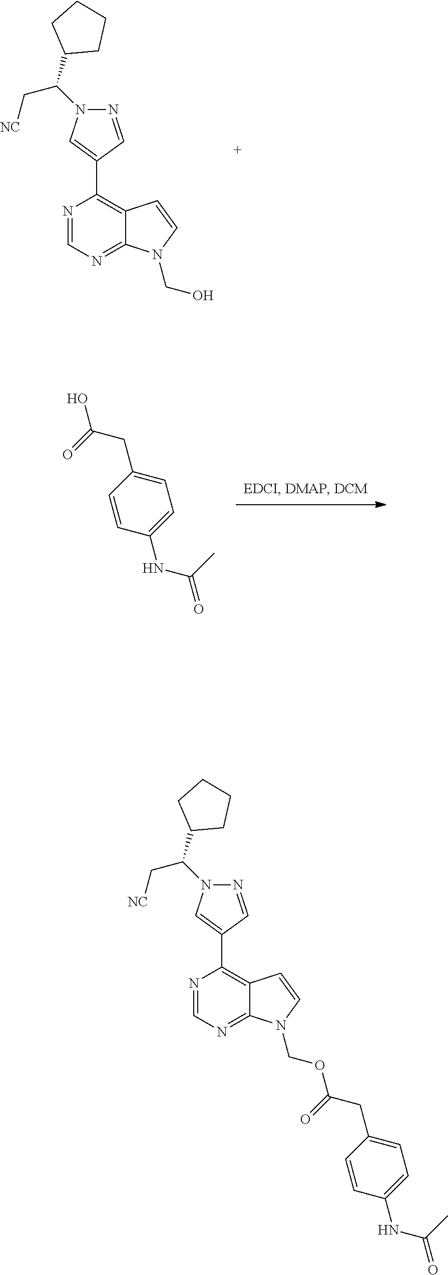 [SMILES: N#CC[C@H](C1CCCC1)n1cc(-c2ncnc3c2ccn3CO)cn1 ; CC(=O)Nc1ccc(CC(=O)O)cc1 ; CC(=O)Nc1ccc(CC(=O)OCn2ccc3c(-c4cnn([C@H](CC#N)C5CCCC5)c4)ncnc32)cc1]

### Synthesis of (R)-(4-(1-(2-cyano-1-cyclopentylethyl)-1H-pyrazol-4-yl)-7H-pyrrolo [2, 3-d] pyrimidin-7-yl) methyl 2-(4-acetamidophenyl) acetate

(R)-3-cyclopentyl-3-(4-(7-(hydroxymethyl)-7H-pyrrolo [2, 3-d] pyrimidin-4-yl)-1H-pyrazol-1-yl) propanenitrile (171 mg, 0.5 mmol), 4-dimethylamino pyridine (DMAP, 61 mg, 0.5 mmol), 2-(4-acetamidophenyl) acetic acid (actarit, 145 mg, 0.75 mmol) and 1-(3-dimethylaminopropyl)-3-ethylcarbodiimide hydrochloride (EDCI, 144 mg, 0.75 mmol) were dissolved in a mixed solvent of dichloromethane (8.5 mL) and N, N-dimethylformamide (0.3 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.133 g, 52% yield. MS (m/z): [M+H]⁺ calcd for C₂₈H₂₉N₇O₃, 512.23; found, 512.3.1H NMR (400 MHz, DMSO-d₆) δ 9.91 (s, 1H), 8.83 (d, J=20.0 Hz, 2H), 8.41 (s, 1H), 7.75 (d, J=3.7 Hz, 1H), 7.49 (d, J=8.3 Hz, 2H), 7.18-7.10 (m, 3H), 6.27 (s, 2H), 4.55 (td, J=9.6, 4.2 Hz, 1H), 3.65 (s, 2H), 3.30-3.15 (m, 2H), 2.48-2.37 (m, 1H), 2.03 (s, 3H), 1.87-1.76 (m, 1H), 1.66-1.42 (m, 4H), 1.41-1.15 (m, 3H).

### Example 111

### (4-(1-((R)-2-cyano-1-cyclopentylethyl)-1H-pyrazol-4-yl)-7H-pyrrolo [2, 3-d] pyrimidin-7-yl) methyl (S)-2-((tert-butoxycarbonyl) amino)-2-phenylacetate

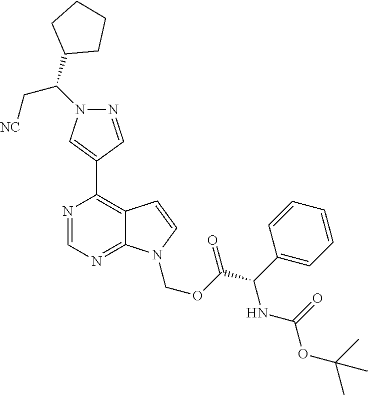 [SMILES: CC(C)(C)OC(=O)N[C@H](C(=O)OCn1ccc2c(-c3cnn([C@H](CC#N)C4CCCC4)c3)ncnc21)c1ccccc1]

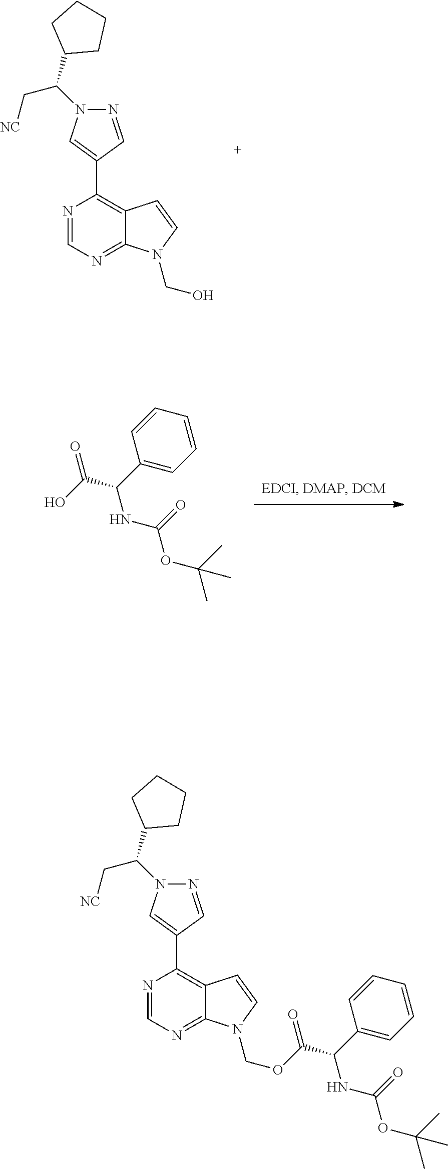 [SMILES: N#CC[C@H](C1CCCC1)n1cc(-c2ncnc3c2ccn3CO)cn1 ; CC(C)(C)OC(=O)N[C@H](C(=O)O)c1ccccc1 ; CC(C)(C)OC(=O)N[C@H](C(=O)OCn1ccc2c(-c3cnn([C@H](CC#N)C4CCCC4)c3)ncnc21)c1ccccc1]

### Synthesis of (4-(1-((R)-2-cyano-1-cyclopentylethyl)-1H-pyrazol-4-yl)-7H-pyrrolo [2, 3-d] pyrimidin-7-yl) methyl (S)-2-((tert-butoxycarbonyl) amino)-2-phenylacetate

(R)-3-cyclopentyl-3-(4-(7-(hydroxymethyl)-7H-pyrrolo [2, 3-d] pyrimidin-4-yl)-1H-pyrazol-1-yl) propanenitrile (171 mg, 0.5 mmol), 4-dimethylaminopyridine (DMAP, 61 mg, 0.5 mmol), N-Boc-L-phenylglycine (189 mg, 0.75 mmol) and 1-(3-dimethylaminopropyl)-3-ethylcarbodiimide hydrochloride (EDCI, 144 mg, 0.75 mmol) were dissolved in a mixed solvent of dichloromethane (8.5 mL) and N, N-dimethylformamide (0.3 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.153 g, 53.7% yield. MS (m/z): [M+H]⁺ calcd for C₃₁H₃₅N₇O₄, 570.28; found, 570.2. ¹H NMR (400 MHz, DMSO-d₆) δ 8.84 (s, 1H), 8.75 (s, 1H), 8.40 (s, 1H), 7.82 (d, J=7.8 Hz, 1H), 7.66 (d, J=3.7 Hz, 1H), 7.40-7.19 (m, 5H), 7.09 (d, J=3.8 Hz, 1H), 6.37-6.22 (m, 2H), 5.19 (d, J=7.8 Hz, 1H), 4.55 (td, J=9.7, 4.2 Hz, 1H), 3.29-3.15 (m, 2H), 2.45-2.34 (m, 1H), 1.88-1.77 (m, 1H), 1.67-1.40 (m, 4H), 1.39-1.11 (m, 12H).

### Example 112

### Methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-(4-isobutylphenyl) propanoate

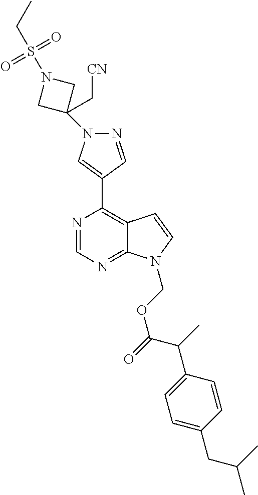 [SMILES: CCS(=O)(=O)N1CC(CC#N)(n2cc(-c3ncnc4c3ccn4COC(=O)C(C)c3ccc(CC(C)C)cc3)cn2)C1]

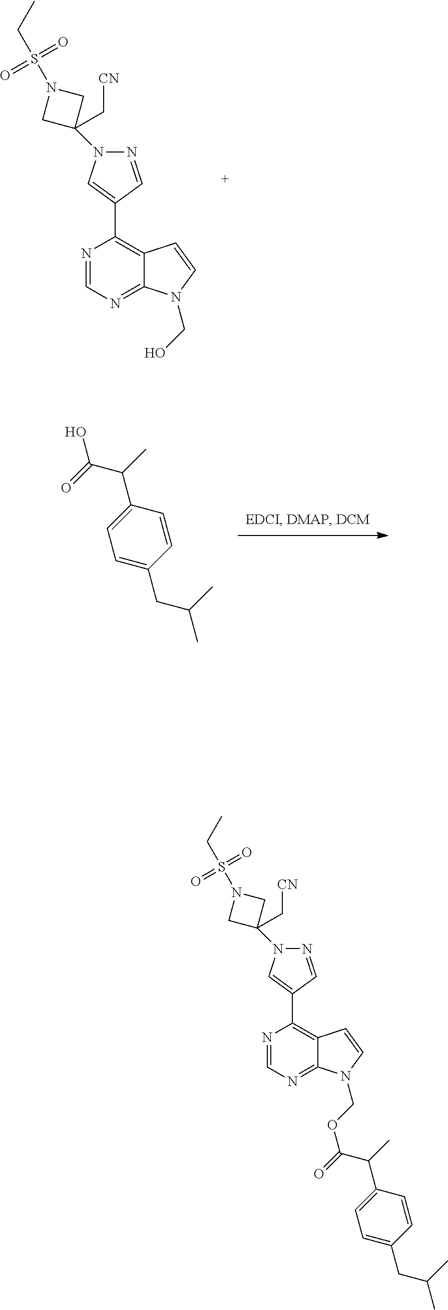 [SMILES: CCS(=O)(=O)N1CC(CC#N)(n2cc(-c3ncnc4c3ccn4CO)cn2)C1 ; CC(C)Cc1ccc(C(C)C(=O)O)cc1 ; CCS(=O)(=O)N1CC(CC#N)(n2cc(-c3ncnc4c3ccn4COC(=O)C(C)c3ccc(CC(C)C)cc3)cn2)C1]

### Synthesis of methyl (4-(1-(3-(cyanomethyl)-1-(ethylsulfonyl) azetidin-3-yl)-1H-pyrazol-4-yl)-7H-pyrrolo [2, 3-d] pyrimidin-7-yl) 2-(4-isobutylphenyl) propanoate

2-(1-(Ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile (143 mg, 0.356 mmol), 4-dimethylamino pyridine (DMAP, 44 mg, 0.356 mmol), 2-(4-isobutylphenyl) propanoic acid (ibuprofen, 110 mg, 0.534 mmol) and 1-(3-dimethylaminopropyl)-3-ethylcarbodiimide hydrochloride (EDCI, 103 mg, 0.534 mmol) were dissolved in dichloromethane (3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.191 g, 91% yield. MS (m/z): [M+H]⁺ calcd for C₃₀H₃₅N₇O₄S, 590.25, found 590.3. ¹H NMR (400 MHz, Chloroform-d) δ 8.85 (s, 1H), 8.49 (s, 1H), 8.31 (s, 1H), 7.44 (d, J=3.8 Hz, 1H), 7.09 (d, J=8.1 Hz, 2H), 7.00 (d, J=8.1 Hz, 2H), 6.71 (d, J=3.8 Hz, 1H), 6.27-6.17 (m, 2H), 4.63 (d, J=9.2 Hz, 2H), 4.29-4.22 (m, 2H), 3.70 (q, J=7.1 Hz, 1H), 3.40 (s, 2H), 3.08 (q, J=7.4 Hz, 2H), 2.38 (d, J=7.2 Hz, 2H), 1.79 (dh, J=13.5, 6.7 Hz, 1H), 1.48-1.38 (m, 6H), 0.85 (d, J=6.6 Hz, 6H).

### Example 113

### (4-(Methyl ((trans)-4-((N-methylsulfamoyl) methyl) cyclohexyl) amino)-7H-pyrrolo [2, 3-d] pyrimidin-7-yl) methyl 2-(2-fluoro-[1, 1′-biphenyl]-4-yl) propanoate

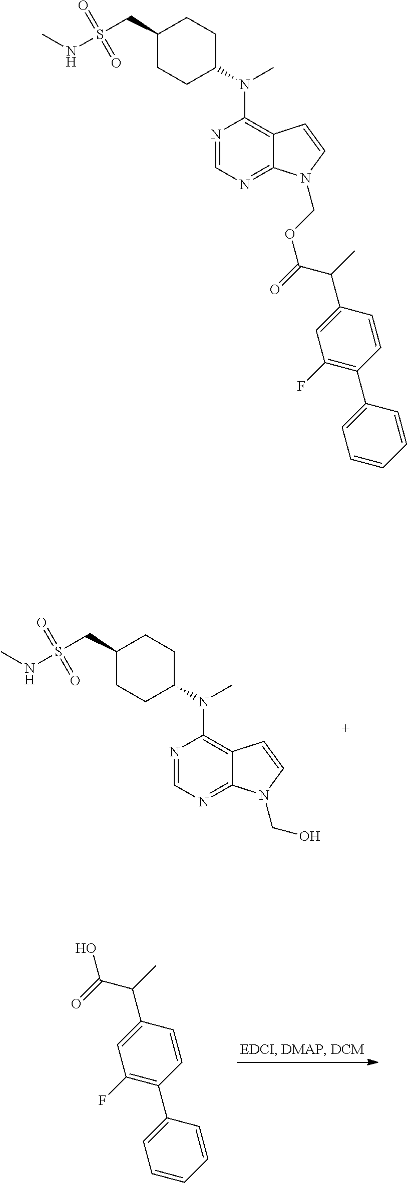

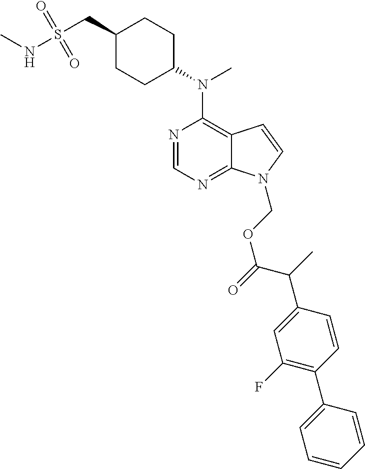

### Synthesis of (4-(methyl ((trans)-4-((N-methylsulfamoyl) methyl) cyclohexyl) amino)-7H-pyrrolo [2, 3-d] pyrimidin-7-yl) methyl 2-(2-fluoro-[1, 1′-biphenyl]-4-yl) propanoate

1-((Trans)-4-((7-(hydroxymethyl)-7H-pyrrolo [2, 3-d] pyrimidin-4-yl) (methyl) amino) cyclohexyl)-N-methyl methanesulfonamide (170 mg, 0.5 mmol), 4-dimethylamino pyridine (DMAP, 61 mg, 0.5 mmol), 2-(2-fluoro-4-biphenyl) propanoic acid (flurbiprofen, 183 mg, 0.75 mmol) and 1-(3-dimethylaminopropyl)-3-ethylcarbodiimide hydrochloride (EDCI, 144 mg, 0.75 mmol) were dissolved in dichloromethane (8.5 mL) and dimethylformamide (0.3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.072 g, 24.2% yield. MS (m/z): [M+H]⁺ calcd for C₃₁H₃₆FN₅O₄S, 594.25; found, 594.1. ¹H NMR (400 MHz, DMSO-d₆) δ 8.16 (s, 1H), 7.54-7.44 (m, 4H), 7.42-7.35 (m, 2H), 7.29 (d, J=3.7 Hz, 1H), 7.12 (d, J=9.9 Hz, 2H), 6.90 (q, J=5.0 Hz, 1H), 6.63 (d, J=3.7 Hz, 1H), 6.17 (d, J=2.1 Hz, 2H), 4.64 (s, 1H), 3.89 (q, J=7.1 Hz, 1H), 3.15 (s, 3H), 2.95 (d, J=6.2 Hz, 2H), 2.59 (d, J=5.0 Hz, 3H), 2.07-1.99 (m, 2H), 1.84 (dt, J=11.8, 6.9 Hz, 1H), 1.75-1.61 (m, 4H), 1.39 (d, J=7.1 Hz, 3H), 1.36-1.21 (m, 2H).

### Example 114

### (4-(Methyl ((trans)-4-((N-methylsulfamoyl) methyl) cyclohexyl) amino)-7H-pyrrolo [2, 3-d] pyrimidin-7-yl) methyl 2-(3-phenoxyphenyl) propanoate

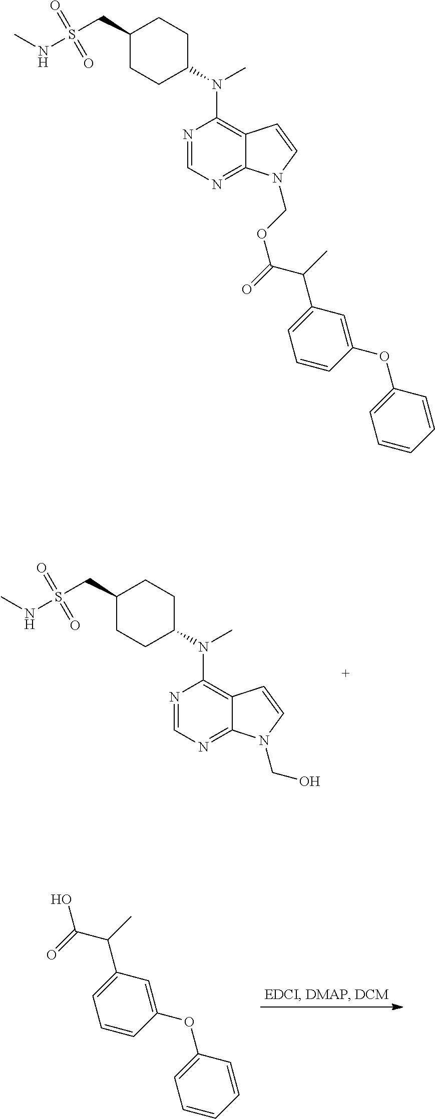

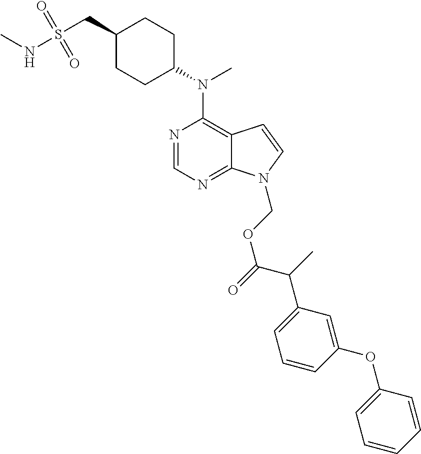

### Synthesis of (4-(methyl ((trans)-4-((N-methylsulfamoyl) methyl) cyclohexyl) amino)-7H-pyrrolo [2, 3-d] pyrimidin-7-yl) methyl 2-(3-phenoxyphenyl) propanoate

1-((Trans)-4-((7-(hydroxymethyl)-7H-pyrrolo [2, 3-d] pyrimidin-4-yl) (methyl) amino) cyclohexyl)-N-methyl methanesulfonamide (170 mg, 0.5 mmol), 4-dimethylaminopyridine (DMAP, 61 mg, 0.5 mmol), 2-(3-phenoxyphenyl) propanoic acid (fenoprofen, 182 mg, 0.75 mmol) and 1-(3-dimethylaminopropyl)-3-ethylcarbodiimide hydrochloride (EDCI, 144 mg, 0.75 mmol) were dissolved in dichloromethane (8.5 mL) and dimethylformamide (0.3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.118 g, 39.9% yield. MS (m/z): [M+H]⁺ calcd for C₃₁H₃₇N₅O₅S, 592.25; found, 592.2. ¹H NMR (400 MHz, DMSO-d₆) δ 8.22 (s, 1H), 7.42 (dd, J=8.5, 7.3 Hz, 2H), 7.35-7.28 (m, 2H), 7.19 (t, J=7.4 Hz, 1H), 7.07-6.85 (m, 6H), 6.68 (d, J=3.7 Hz, 1H), 6.19 (s, 2H), 4.71 (s, 1H), 3.86 (q, J=7.1 Hz, 1H), 3.21 (s, 3H), 3.01 (d, J=6.2 Hz, 2H), 2.64 (d, J=5.0 Hz, 3H), 2.10 (d, J=12.9 Hz, 2H), 1.97-1.82 (m, 1H), 1.73 (t, J=5.3 Hz, 4H), 1.42-1.27 (m, 5H).

### Example 115

### (4-(Methyl ((trans)-4-((N-methylsulfamoyl) methyl) cyclohexyl) amino)-7H-pyrrolo [2, 3-d] pyrimidin-7-yl) methyl 2-(4-((2-oxocyclopentyl) methyl) phenyl) propanoate

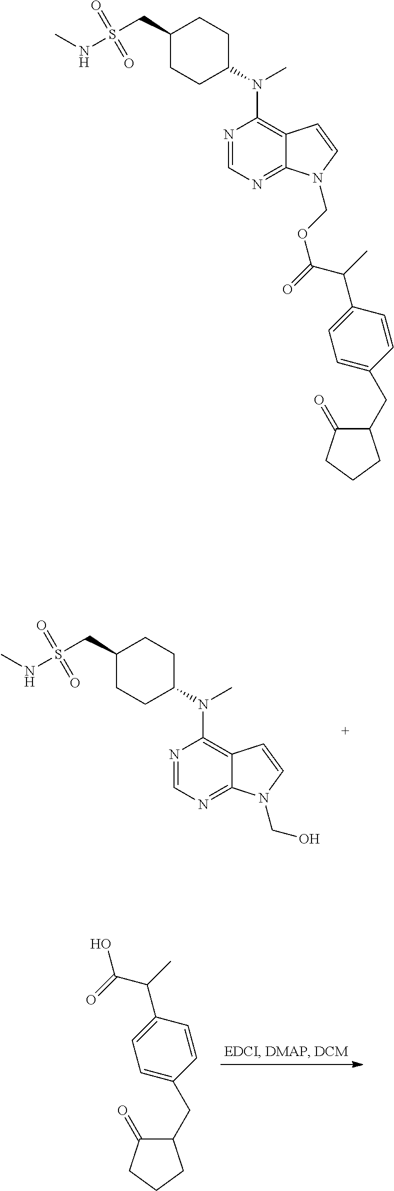

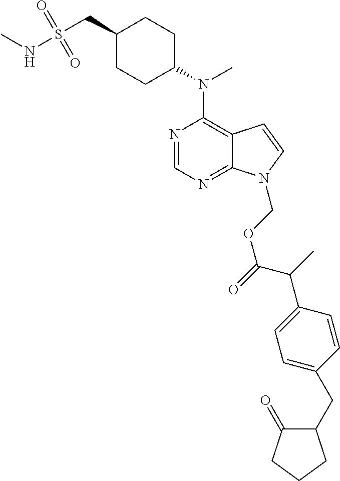

### Synthesis of (4-(methyl ((trans)-4-((N-methylsulfamoyl) methyl) cyclohexyl) amino)-7H-pyrrolo [2, 3-d] pyrimidin-7-yl) methyl 2-(4-((2-oxocyclopentyl) methyl) phenyl) propanoate

1-((Trans)-4-((7-(hydroxymethyl)-7H-pyrrolo [2, 3-d] pyrimidin-4-yl) (methyl) amino) cyclohexyl)-N-methyl methanesulfonamide (170 mg, 0.5 mmol), 4-dimethylamino pyridine (DMAP, 61 mg, 0.5 mmol), 2-[4-(2-oxocyclopentan-1-ylmethyl) phenyl] propanoic acid (loxoprofen, 185 mg, 0.75 mmol) and 1-(3-dimethylaminopropyl)-3-ethylcarbodiimide hydrochloride (EDCI, 144 mg, 0.75 mmol) were dissolved in dichloromethane (8.5 mL) and dimethylformamide (0.3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.073 g, 24.5% yield. MS (m/z): [M+H]⁺ calcd for C₃₁H₄₁N₅O₅S, 596.28; found, 596.2. ¹H NMR (400 MHz, DMSO-d₆) δ 8.15 (d, J=1.9 Hz, 1H), 7.25 (d, J=3.7 Hz, 1H), 7.07 (qd, J=8.3, 2.9 Hz, 4H), 6.89 (q, J=4.9 Hz, 1H), 6.62 (d, J=3.8 Hz, 1H), 6.12 (d, J=1.6 Hz, 2H), 4.65 (s, 1H), 3.74 (q, J=7.0 Hz, 1H), 3.16 (s, 3H), 3.00-2.80 (m, 3H), 2.59 (d, J=5.0 Hz, 3H), 2.45-2.16 (m, 3H), 2.05 (dddd, J=14.5, 10.1, 8.5, 1.7 Hz, 3H), 1.93-1.78 (m, 3H), 1.74-1.60 (m, 5H), 1.48-1.41 (m, 1H), 1.36-1.21 (m, 5H).

### Example 116

### (4-(Methyl ((trans)-4-((N-methylsulfamoyl) methyl) cyclohexyl) amino)-7H-pyrrolo [2, 3-d] pyrimidin-7-yl) methyl 2-(1-(4-chlorobenzoyl)-5-methoxy-2-methyl-1H-indol-3-yl) acetate

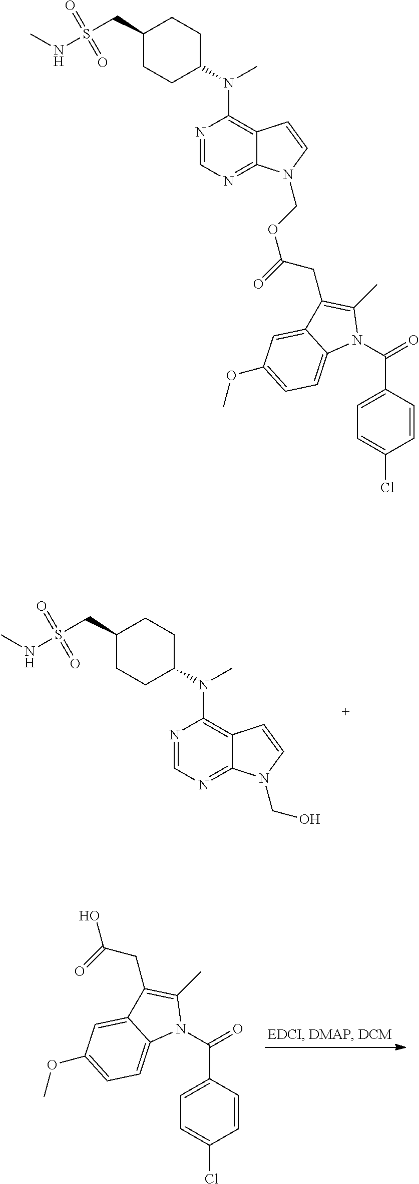

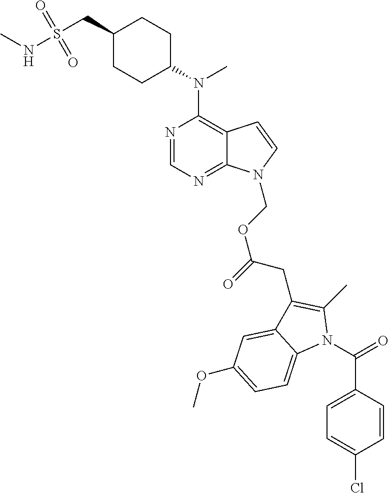

### Synthesis of (4-(Methyl ((trans)-4-((N-methylsulfamoyl) methyl) cyclohexyl) amino)-7H-pyrrolo [2, 3-d] pyrimidin-7-yl) methyl 2-(1-(4-chlorobenzoyl)-5-methoxy-2-methyl-1H-indol-3-yl) acetate

1-((Trans)-4-((7-(hydroxymethyl)-7H-pyrrolo [2, 3-d] pyrimidin-4-yl) (methyl) amino) cyclohexyl)-N-methyl methanesulfonamide (170 mg, 0.5 mmol), 4-dimethylamino pyridine (DMAP, 61 mg, 0.5 mmol), 1-(4-chlorobenzoyl)-5-methoxy-2-methyl-1H-indole-3-acetic acid (indomethacin, 277 mg, 0.75 mmol) and 1-(3-dimethylaminopropyl)-3-ethylcarbodiimide hydrochloride (EDCI, 144 mg, 0.75 mmol) were dissolved in dichloromethane (8.5 mL) and dimethylformamide (0.3 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a light yellow solid, 0.136 g, 38.5% yield. MS (m/z): [M+H]⁺ calcd for C₃₅H₃₉ClN₆O₆S, 707.23; found, 707.0. ¹H NMR (400 MHz, DMSO-d₆) δ 8.17 (s, 1H), 7.64 (s, 4H), 7.28 (d, J=3.7 Hz, 1H), 7.01-6.83 (m, 3H), 6.70 (dd, J=9.0, 2.6 Hz, 1H), 6.64 (d, J=3.8 Hz, 1H), 6.17 (s, 2H), 4.67 (s, 1H), 3.80 (s, 2H), 3.70 (s, 3H), 3.17 (s, 3H), 2.96 (d, J=6.2 Hz, 2H), 2.60 (d, J=4.9 Hz, 3H), 2.14 (s, 3H), 2.06 (d, J=12.8 Hz, 2H), 1.96-1.80 (m, 1H), 1.77-1.63 (m, 4H), 1.31 (p, J=4.9, 4.3 Hz, 2H).

### Example 117

### (4-(Methyl ((trans)-4-((N-methylsulfamoyl) methyl) cyclohexyl) amino)-7H-pyrrolo [2, 3-d] pyrimidin-7-yl) methyl 2-(4-(1-) oxoisoindolin-2-yl) phenyl) butanoate

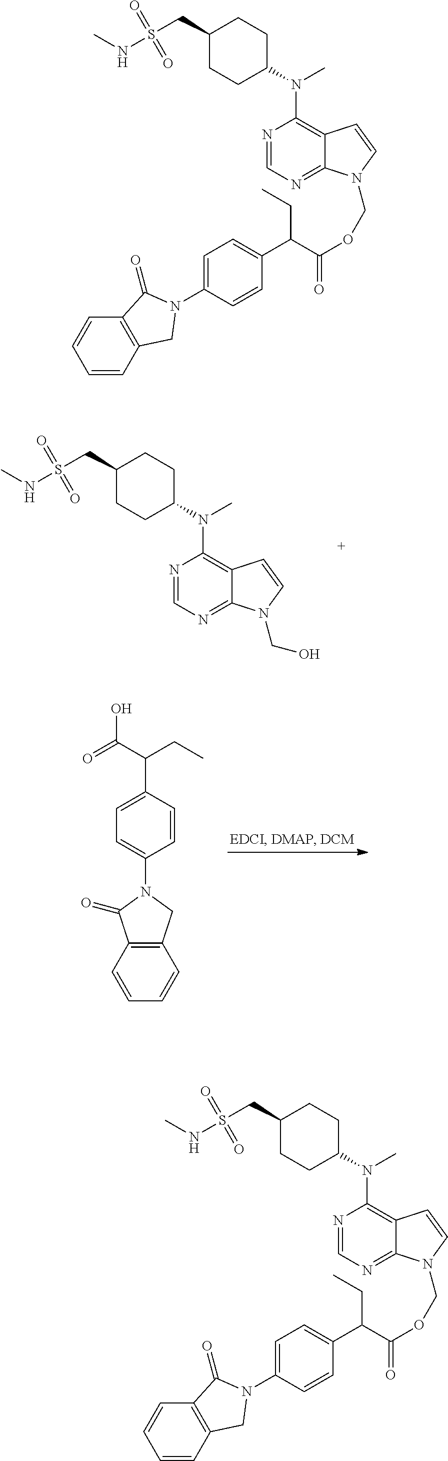

### Synthesis of (4-(methyl ((trans)-4-((N-methylsulfamoyl) methyl) cyclohexyl) amino)-7H-pyrrolo [2, 3-d] pyrimidin-7-yl) methyl 2-(4-(1-) oxoisoindolin-2-yl) phenyl) butanoate

1-((Trans)-4-((7-(hydroxymethyl)-7H-pyrrolo [2, 3-d] pyrimidin-4-yl) (methyl) amino) cyclohexyl)-N-methyl methanesulfonamide (20 mg, 0.058 mmol), 4-dimethylamino pyridine (DMAP, 4 mg, 0.03 mmol), 2-(4-(1-oxoisoindolin-2-yl) phenyl) butanoic acid (indobufen, 26 mg, 0.09 mmol) and 1-(3-dimethylaminopropyl)-3-ethylcarbodiimide hydrochloride (EDCI, 17 mg, 0.09 mmol) were dissolved in a mixed solvent of dichloromethane (0.5 mL) and N, N-dimethylformamide (0.05 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.156 g, 48.4% yield. MS (m/z): [M+H]⁺ calcd for C₃₄H₄₀N₆O₅S, 645.28; found, 645.1. ¹H NMR (400 MHz, DMSO-d₆) δ 8.17 (s, 1H), 7.86-7.75 (m, 3H), 7.68 (dd, J=6.3, 1.2 Hz, 2H), 7.55 (ddd, J=8.1, 6.3, 2.0 Hz, 1H), 7.34-7.24 (m, 3H), 6.88 (q, J=5.0 Hz, 1H), 6.62 (d, J=3.8 Hz, 1H), 6.22-6.08 (m, 2H), 4.99 (s, 2H), 4.64 (s, 1H), 3.55 (t, J=7.6 Hz, 1H), 3.14 (s, 3H), 2.94 (d, J=6.2 Hz, 2H), 2.59 (d, J=5.0 Hz, 3H), 2.06-1.91 (m, 3H), 1.89-1.78 (m, 1H), 1.77-1.61 (m, 5H), 1.33-1.21 (m, 2H), 0.79 (t, J=7.3 Hz, 3H).

### Example 118

### (4-(Methyl ((trans)-4-((N-methylsulfamoyl) methyl) cyclohexyl) amino)-7H-pyrrolo [2, 3-d] pyrimidin-7-yl) methyl 2-((2, 3-dimethylphenyl) amino) benzoate

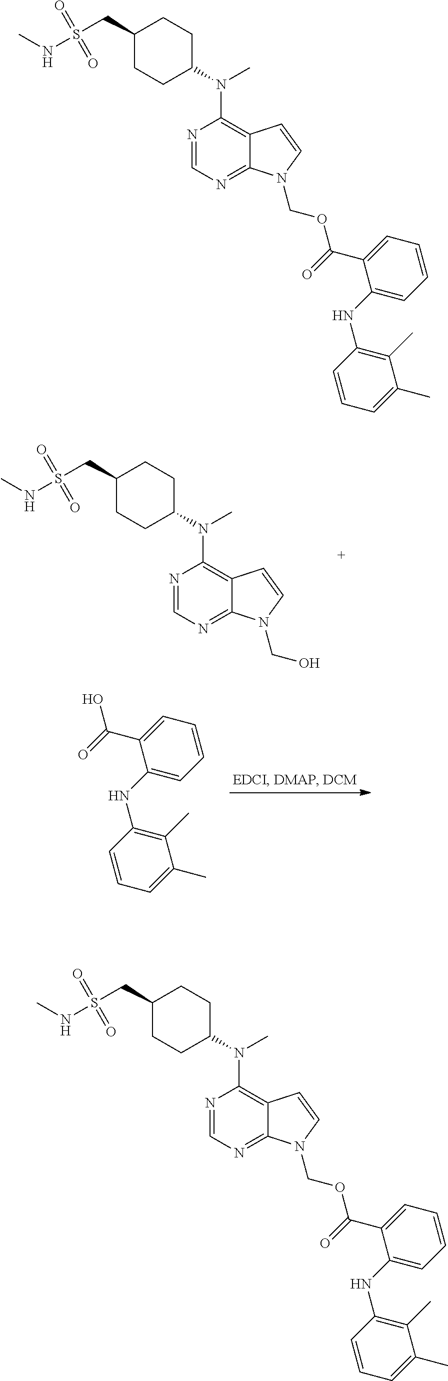

### Synthesis of (4-(methyl ((trans)-4-((N-methylsulfamoyl) methyl) cyclohexyl) amino)-7H-pyrrolo [2, 3-d] pyrimidin-7-yl) methyl 2-((2, 3-dimethylphenyl) amino) benzoate

1-((Trans)-4-((7-(hydroxymethyl)-7H-pyrrolo [2, 3-d] pyrimidin-4-yl) (methyl) amino) cyclohexyl)-N-methyl methanesulfonamide (171 mg, 0.5 mmol), 4-dimethylaminopyridine (DMAP, 61 mg, 0.5 mmol), 2-((2, 3-dimethylphenyl) amino) benzoic acid (mefenamic acid, 181 mg, 0.75 mmol) and 1-(3-dimethylaminopropyl)-3-ethylcarbodiimide hydrochloride (EDCI, 144 mg, 0.75 mmol) were dissolved in a mixed solvent of dichloromethane (8.5 mL) and N, N-dimethylformamide (0.3 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a yellow solid, 0.096 g, 32.5% yield. MS (m/z): [M+H]⁺ calcd for C₃₁H₃₈N₆O₄S, 591.27; found, 591.2. H NMR (400 MHz, DMSO-d₆) δ 9.07 (s, 1H), 8.21 (s, 1H), 7.73 (dd, J=8.1, 1.6 Hz, 1H), 7.47 (dd, J=8.8, 3.9 Hz, 1H), 7.32 (ddd, J=8.6, 7.0, 1.7 Hz, 1H), 7.19-7.03 (m, 3H), 6.90 (q, J=4.9 Hz, 1H), 6.66 (ddd, J=20.2, 9.1, 4.5 Hz, 3H), 6.41 (s, 2H), 4.68 (s, 1H), 3.22-3.13 (m, 3H), 2.95 (d, J=6.2 Hz, 2H), 2.59 (d, J=4.9 Hz, 3H), 2.29 (s, 3H), 2.06 (d, J=14.3 Hz, 5H), 1.92-1.79 (m, 1H), 1.70 (h, J=3.4 Hz, 4H), 1.38-1.19 (m, 2H).

### Example 119

### (4-(Methyl ((trans)-4-((N-methylsulfamoyl) methyl) cyclohexyl) amino)-7H-pyrrolo [2, 3-d] pyrimidin-7-yl) methyl 2-(4-isobutylphenyl) propanoate

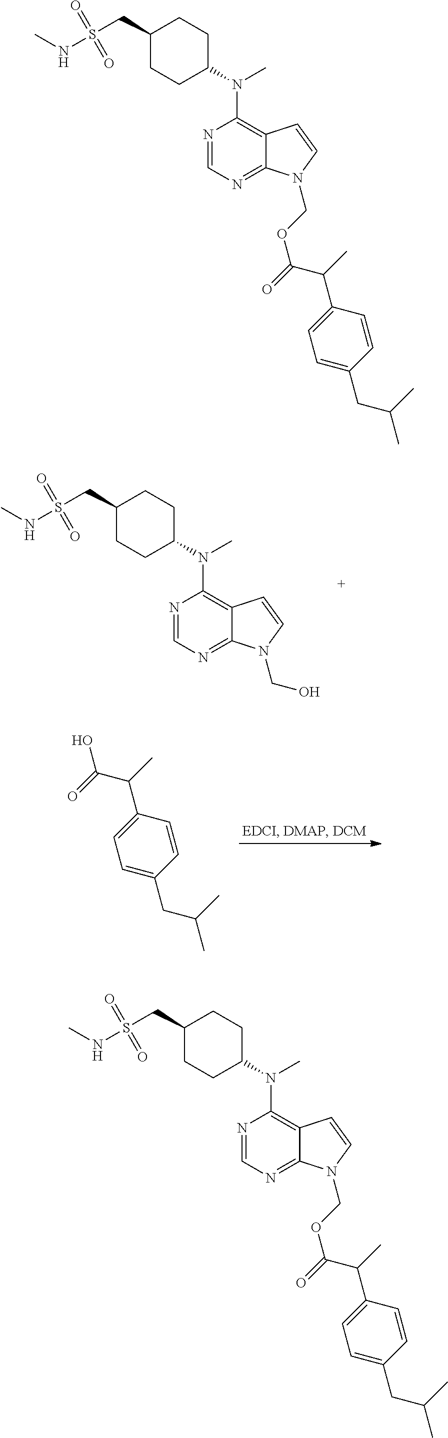

### Synthesis of (4-(methyl ((trans)-4-((N-methylsulfamoyl) methyl) cyclohexyl) amino)-7H-pyrrolo [2, 3-d] pyrimidin-7-yl) methyl 2-(4-isobutylphenyl) propanoate

1-((Trans)-4-((7-(hydroxymethyl)-7H-pyrrolo [2, 3-d] pyrimidin-4-yl) (methyl) amino) cyclohexyl)-N-methyl methanesulfonamide (201 mg, 0.5 mmol), 4-dimethylamino pyridine (DMAP, 61 mg, 0.5 mmol), 2-(4-isobutylphenyl) propanoic acid (ibuprofen, 123.6 mg, 0.6 mmol) and 1-(3-dimethylaminopropyl)-3-ethylcarbodiimide hydrochloride (EDCI, 144 mg, 0.75 mmol) were dissolved in dichloromethane (10 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a white solid, 0.142 g, 51.1% yield. MS (m/z): [M+H]⁺ calcd for C₂₉H₄₁N₅O₄S, 556.29, found 556.21H NMR (400 MHz, DMSO-d₆) δ 8.15 (s, 1H), 7.24 (d, J=3.8 Hz, 1H), 7.08 (d, J=8.1 Hz, 2H), 7.01 (d, J=8.0 Hz, 2H), 6.89 (q, J=5.0 Hz, 1H), 6.61 (d, J=3.7 Hz, 1H), 6.12 (s, 2H), 4.66 (s, 1H), 3.73 (q, J=7.0 Hz, 1H), 3.15 (s, 3H), 2.95 (d, J=6.2 Hz, 2H), 2.59 (d, J=5.0 Hz, 3H), 2.37 (d, J=7.1 Hz, 2H), 2.05 (d, J=12.9 Hz, 2H), 1.91-1.80 (m, 1H), 1.79-1.64 (m, 5H), 1.37-1.25 (m, 5H), 0.82 (d, J=6.6 Hz, 6H).

### Example 120

(4-(Methyl ((trans)-4-((N-methylsulfamoyl)methyl) cyclohexyl) amino)-7H-pyrrolo[2, 3-d] pyrimidin-7-yl) methyl 2-((3-chloro-2-methylphenyl)amino) benzoate

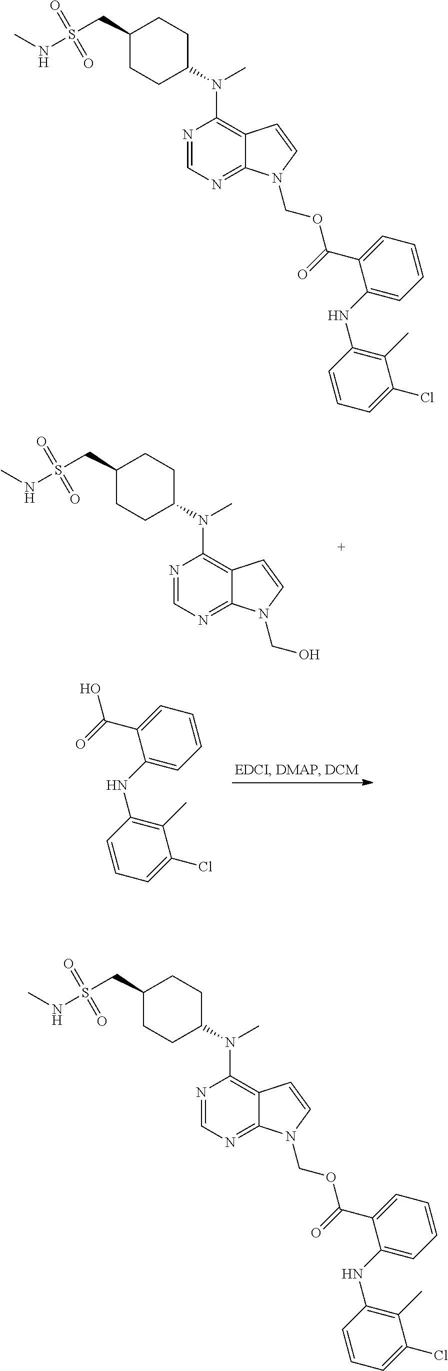

### Synthesis of (4-(methyl ((trans)-4-((N-methylsulfamoyl) methyl) cyclohexyl) amino)-7H-pyrrolo [2, 3-d] pyrimidin-7-yl) methyl 2-((3-chloro-2-methylphenyl) amino) benzoate

1-((Trans)-4-((7-(hydroxymethyl)-7H-pyrrolo [2, 3-d] pyrimidin-4-yl) (methyl) amino) cyclohexyl)-N-methyl methanesulfonamide (200 mg, 0.58 mmol), 4-dimethylamino pyridine (DMAP, 35 mg, 0.29 mmol), 2-((3-chloro-2-methylphenyl) amino) benzoic acid (tolfenamic acid, 230 mg, 0.88 mmol) and 1-(3-dimethylaminopropyl)-3-ethylcarbodiimide hydrochloride (EDCI, 168 mg, 0.88 mmol) were dissolved in a mixed solvent of dichloromethane (10 mL) and N, N-dimethylformamide (0.2 mL), and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=150:1 to 50:1) to give the title compound as a yellow solid, 0.137 g, 44.9% yield. MS (m/z): [M+H]⁺ calcd for C₃₀H₃₅ClN₆O₄S, 611.21; found, 611.2. ¹H NMR (400 MHz, DMSO-d₆) δ 9.18 (s, 1H), 8.26 (s, 1H), 7.81 (dd, J=8.2, 1.5 Hz, 1H), 7.51 (d, J=3.7 Hz, 1H), 7.43 (td, J=7.7, 7.1, 1.7 Hz, 1H), 7.40-7.20 (m, 3H), 6.95 (q, J=5.0 Hz, 1H), 6.84-6.73 (m, 3H), 6.47 (s, 2H), 4.74 (s, 1H), 3.24 (s, 3H), 3.01 (d, J=6.2 Hz, 2H), 2.65 (d, J=4.9 Hz, 3H), 2.29 (s, 3H), 2.11 (dd, J=10.1, 5.0 Hz, 2H), 2.00-1.86 (m, 1H), 1.74 (d, J=9.5 Hz, 4H), 1.36 (q, J=10.0, 7.2 Hz, 2H).

### Example 121

### 4-((4-(1-(3-(Cyanomethyl)-1-(ethylsulfonyl) azetidin-3-yl)-1H-pyrazol-4-yl)-7H-pyrrolo [2, 3-d] pyrimidin-7-yl) methoxy)-2-methyl-N-(pyridin-2-yl)-2H-benzo [e] [1, 2] thiazine-3-carboxamide 1, 1-dioxide

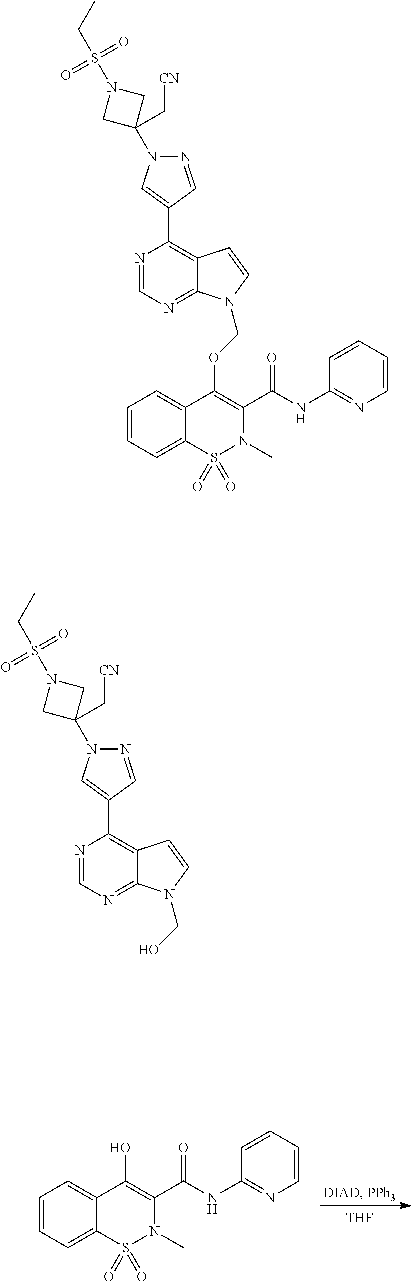 [SMILES: CCS(=O)(=O)N1CC(CC#N)(n2cc(-c3ncnc4c3ccn4COC3=C(C(=O)Nc4ccccn4)N(C)S(=O)(=O)c4ccccc43)cn2)C1 ; CCS(=O)(=O)N1CC(CC#N)(n2cc(-c3ncnc4c3ccn4CO)cn2)C1 ; CN1C(C(=O)Nc2ccccn2)=C(O)c2ccccc2S1(=O)=O ; "THF"]

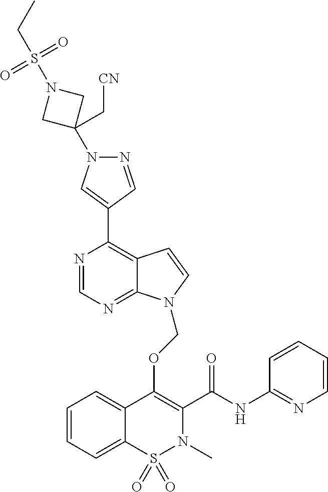 [SMILES: CCS(=O)(=O)N1CC(CC#N)(n2cc(-c3ncnc4c3ccn4COC3=C(C(=O)Nc4ccccn4)N(C)S(=O)(=O)c4ccccc43)cn2)C1]

### Synthesis of 4-((4-(1-(3-(Cyanomethyl)-1-(ethylsulfonyl) azetidin-3-yl)-1H-pyrazol-4-yl)-7H-pyrrolo [2, 3-d] pyrimidin-7-yl) methoxy)-2-methyl-N-(pyridin-2-yl)-2H-benzo [e] [1, 2] thiazine-3-carboxamide 1, 1-dioxide

4-Hydroxy-2-methyl-N-(pyridin-2-yl)-2H-benzo [e] [1, 2] thiazine-3-carboxamide 1, 1-dioxide (piroxicam, 103 mg, 0.312 mmol) and triphenylphosphine (PPh₃, 164 mg, 0.624 mmol) were added to tetrahydrofuran (3 mL) under nitrogen. After cooling to −10° C., diisopropyl azodicarboxylate (DIAD, 95 mg, 0.468 mmol) was added dropwise to the mixture with stirring. After stirring at −10° C. for 20 minutes, the mixture was allowed to warm to room temperature naturally, 2-(1-(Ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile (150 mg, 0.374 mmol) was added and the stirring was continued. The reaction was monitored by TLC. After complete exhaust of the starting material (1 h), the solvent was evaporated under reduced pressure to give the crude product. The crude product was further separated by silica gel column chromatography (dichloromethane/acetonitrile=20:1 to 9:1) to give the title compound as a yellow solid, 0.012 g, 5.3% yield. MS (m/z): [M+H]⁺ calcd for C₃₂H₃₀N₁₀O₆S₂, 715.18, found 715.2.1H NMR (400 MHz, Chloroform-d) δ 8.79 (s, 1H), 8.48 (s, 1H), 8.24 (s, 1H), 8.12 (s, 1H), 7.94-7.85 (m, 2H), 7.76-7.68 (m, 2H), 7.51 (d, J=8.3 Hz, 1H), 7.38-7.32 (m, 1H), 7.24 (d, J=3.8 Hz, 1H), 6.92-6.86 (m, 1H), 6.60 (d, J=3.8 Hz, 1H), 6.05 (s, 2H), 4.56 (d, J=9.3 Hz, 2H), 4.18 (d, J=9.3 Hz, 2H), 3.35 (s, 2H), 3.08-2.96 (m, 5H), 1.36 (t, J=7.4 Hz, 3H).

### Example 122

### 4-((4-(1-(3-(Cyanomethyl)-1-(ethylsulfonyl) azetidin-3-yl)-1H-pyrazol-4-yl)-7H-pyrrolo [2, 3-d] pyrimidin-7-yl) methoxy)-2-methyl-N-(5-methylthiazol-2-yl)-2H-benzo [e] [1, 2] thiazine-3-carboxamide 1, 1-dioxide

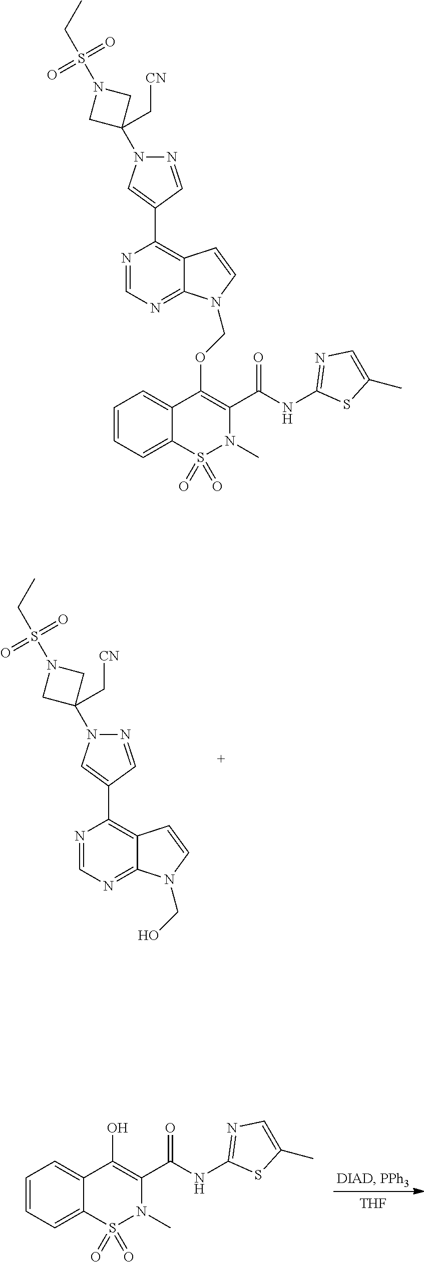 [SMILES: CCS(=O)(=O)N1CC(CC#N)(n2cc(-c3ncnc4c3ccn4COC3=C(C(=O)Nc4ncc(C)s4)N(C)S(=O)(=O)c4ccccc43)cn2)C1 ; CCS(=O)(=O)N1CC(CC#N)(n2cc(-c3ncnc4c3ccn4CO)cn2)C1 ; Cc1cnc(NC(=O)C2=C(O)c3ccccc3S(=O)(=O)N2C)s1 ; "THF"]

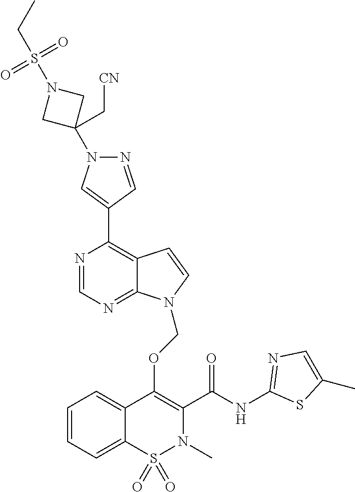 [SMILES: CCS(=O)(=O)N1CC(CC#N)(n2cc(-c3ncnc4c3ccn4COC3=C(C(=O)Nc4ncc(C)s4)N(C)S(=O)(=O)c4ccccc43)cn2)C1]

### Synthesis of 4-((4-(1-(3-(Cyanomethyl)-1-(ethylsulfonyl) azetidin-3-yl)-1H-pyrazol-4-yl)-7H-pyrrolo [2, 3-d] pyrimidin-7-yl) methoxy)-2-methyl-N-(5-methylthiazol-2-yl)-2H-benzo [e] [1, 2] thiazine-3-carboxamide 1, 1-dioxide

4-Hydroxy-2-methyl-N-(5-methylthiazol-2-yl)-2H-benzo [e] [1, 2] thiazine-3-carboxamide 1, 1-dioxide (meloxicam, 147 mg, 0.42 mmol) and triphenylphosphine (PPh₃, 137 mg, 0.52 mmol) were added to tetrahydrofuran (2.8 mL) under nitrogen. Diisopropyl azodicarboxylate (DIAD, 92 mg, 0.45 mmol) was added dropwise to the mixture with stirring. After stirring at −10° C. for 20 minutes, the mixture was allowed to warm to room temperature naturally. 2-(1-(Ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile (140 mg, 0.35 mmol) was added and the stirring was continued. The reaction was monitored by TLC. After complete exhaust of the starting material (1 h), the solvent was evaporated under reduced pressure to give the crude product. The crude product was further separated by silica gel column chromatography (dichloromethane/acetonitrile=20:1 to 9:1) to give the title compound as a yellow solid, 0.005 g, 1.9% yield. MS (m/z): [M+H]⁺ calcd for C₃₁H₃₀N₁₀O₆S₃, 735.15, found 735.1. ¹H NMR (400 MHz, DMSO-d₆) δ 14.61 (s, 1H), 8.94 (s, 1H), 8.77 (s, 1H), 8.49 (s, 1H), 8.02 (d, J=7.7 Hz, 1H), 7.88-7.78 (m, 3H), 7.73 (d, J=3.7 Hz, 1H), 7.28 (s, 1H), 7.15 (d, J=3.7 Hz, 1H), 5.62 (s, 2H), 4.60 (d, J=9.1 Hz, 2H), 4.24 (dd, J=9.1, 2.4 Hz, 2H), 3.69 (d, J=2.7 Hz, 2H), 3.27-3.20 (m, 2H), 2.85 (s, 3H), 2.32 (s, 3H), 1.27-1.20 (m, 3H).

### Example 123

### (3S, 4R)-3-ethyl-4-(3-((S)-2-(4-isobutylphenyl) propanoyl)-3H-imidazo [1, 2-a] pyrrolo [2, 3-e] pyrazin-8-yl)-N-(2, 2, 2-trifluoroethyl) pyrrolidine-1-carboxamide

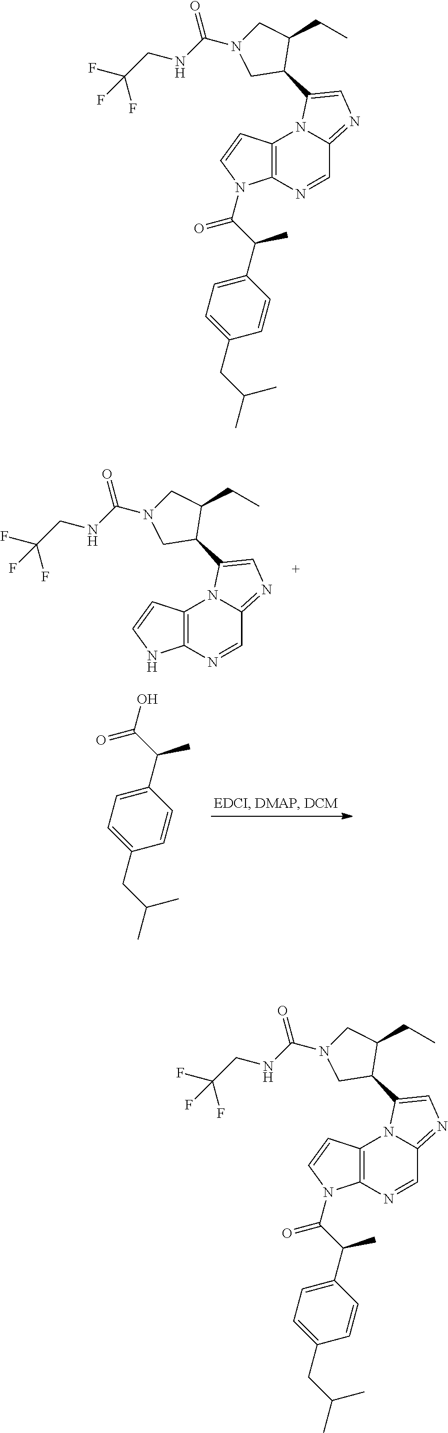

### Synthesis of (3S, 4R)-3-ethyl-4-(3-((S)-2-(4-isobutylphenyl) propanoyl)-3H-imidazo [1, 2-a] pyrrolo [2, 3-e] pyrazin-8-yl)-N-(2, 2, 2-trifluoroethyl) pyrrolidine-1-carboxamide

(3S, 4R)-3-ethyl-4-(3H-imidazo [1, 2-a] pyrrolo [2, 3-e] pyrazin-8-yl)-N-(2, 2, 2-trifluoroethyl) pyrrolidine-1-carboxamide (Upadacitinib, 338 mg, 1 mmol), 4-dimethylamino pyridine (DMAP, 122 mg, 1 mmol), (S)-(+)-2-(4-isobutylphenyl) propanoic acid ((S)-(+)-ibuprofen, 247 mg, 1.2 mmol) and 1-(3-dimethylaminopropyl)-3-ethylcarbodiimide hydrochloride (EDCI, 288 mg, 1.5 mmol) were dissolved in dichloromethane (10 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=50:1 to 25:1) to give the title compound as a white solid, 0.05 g, 17.6% yield. MS (m/z): [M+H]⁺ calcd for C₃₀H₃₅F₃N₆O₂, 569.28; found, 569.3. H NMR (400 MHz, DMSO-d₆) δ 8.88 (s, 1H), 8.04 (d, J=4.2 Hz, 1H), 7.63 (s, 1H), 7.37-7.29 (m, 2H), 7.19-7.04 (m, 3H), 6.97 (t, J=6.3 Hz, 1H), 5.99 (q, J=6.9 Hz, 1H), 4.34 (q, J=6.5 Hz, 1H), 3.91-3.71 (m, 4H), 3.71-3.61 (m, 1H), 3.25 (dd, J=10.2, 5.7 Hz, 1H), 2.38 (dd, J=23.9, 7.1 Hz, 3H), 1.83-1.72 (m, 1H), 1.59 (d, J=6.9 Hz, 3H), 1.07-0.93 (m, 1H), 0.92-0.80 (m, 7H), 0.60 (t, J=7.3 Hz, 3H).

### Example 124

### (3S, 4R)-3-ethyl-4-(3-(2-(4-isobutylphenyl) propanoyl)-3H-imidazo [1, 2-a] pyrrolo [2, 3-e] pyrazin-8-yl)-N-(2, 2, 2-trifluoroethyl) pyrrolidine-1-carboxamide

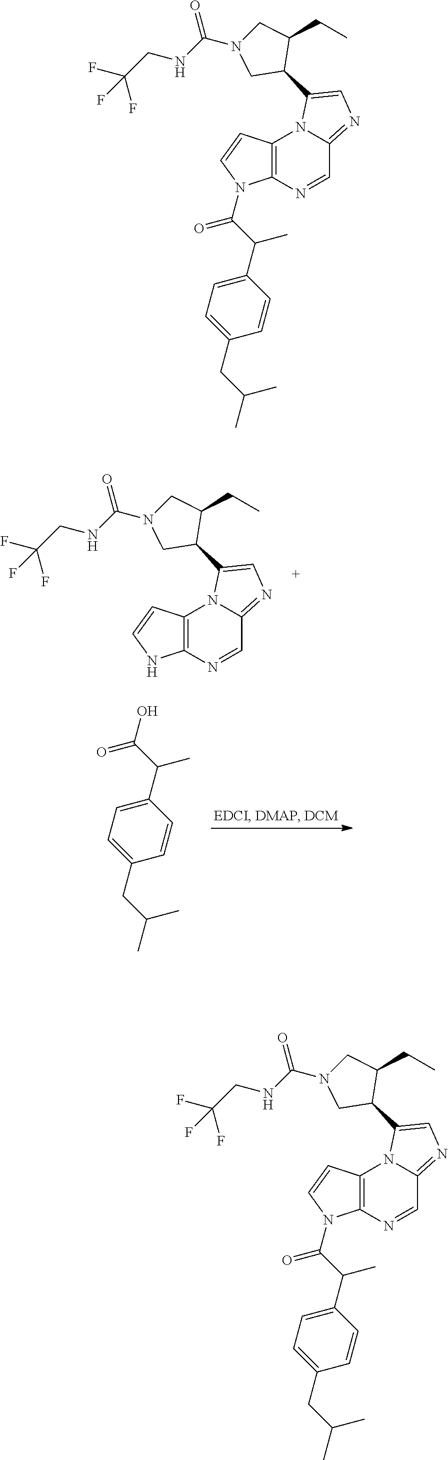 [SMILES: CC[C@@H]1CN(C(=O)NCC(F)(F)F)C[C@@H]1c1cnc2cnc3c(ccn3C(=O)C(C)c3ccc(CC(C)C)cc3)n12 ; CC[C@@H]1CN(C(=O)NCC(F)(F)F)C[C@@H]1c1cnc2cnc3[nH]ccc3n12 ; CC(C)Cc1ccc(C(C)C(=O)O)cc1 ; CC[C@@H]1CN(C(=O)NCC(F)(F)F)C[C@@H]1c1cnc2cnc3c(ccn3C(=O)C(C)c3ccc(CC(C)C)cc3)n12]

### Synthesis of (3S, 4R)-3-ethyl-4-(3-(2-(4-isobutylphenyl) propanoyl)-3H-imidazo [1, 2-a] pyrrolo [2, 3-e] pyrazin-8-yl)-N-(2, 2, 2-trifluoroethyl) pyrrolidine-1-carboxamide

(3S, 4R)-3-ethyl-4-(3H-imidazo [1, 2-a] pyrrolo [2, 3-e] pyrazin-8-yl)-N-(2, 2, 2-trifluoroethyl) pyrrolidine-1-carboxamide (upadacitinib, 100 mg, 0.25 mmol), 4-dimethylamino pyridine (DMAP, 3 mg, 0.025 mmol), 2-(4-isobutylphenyl) propanoic acid (ibuprofen, 54 mg, 0.264 mmol) and 1-(3-dimethylaminopropyl)-3-ethylcarbodiimide hydrochloride (EDCI, 73 mg, 0.377 mmol) were dissolved in dichloromethane (4 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=50:1 to 25:1) to give the title compound as a white solid, 0.097 g, 34.1% yield. MS (m/z): [M+H]⁺ calcd for C₃₀H₃₅F₃N₆O₂, 569.28; found, 569.3. ¹H NMR (400 MHz, Chloroform-d) δ 8.79 (s, 1H), 7.93 (t, J=3.7 Hz, 1H), 7.52 (d, J=4.3 Hz, 1H), 7.36-7.26 (m, 2H), 7.06-6.94 (m, 3H), 6.78 (dd, J=4.2, 1.5 Hz, 1H), 5.98 (qd, J=6.9, 3.4 Hz, 1H), 4.70 (t, J=6.5 Hz, 1H), 4.08 (dt, J=12.2, 6.2 Hz, 1H), 3.98-3.61 (m, 4H), 3.29 (d, J=8.3 Hz, 1H), 2.62-2.46 (m, 1H), 2.31 (dd, J=9.0, 7.2 Hz, 2H), 1.82-1.67 (m, 1H), 1.61 (dd, J=6.9, 1.2 Hz, 3H), 1.27-1.09 (m, 1H), 0.87-0.73 (m, 7H), 0.66 (dt, J=10.3, 7.3 Hz, 3H).

### Example 125

### (3S, 4R)-3-ethyl-4-(3-(2-(2-fluoro-[1, 1′-biphenyl]-4-yl) propanoyl)-3H-imidazo [1, 2-a] pyrrolo [2, 3-e] pyrazin-8-yl)-N-(2, 2, 2-trifluoroethyl) pyrrolidine-1-carboxamide

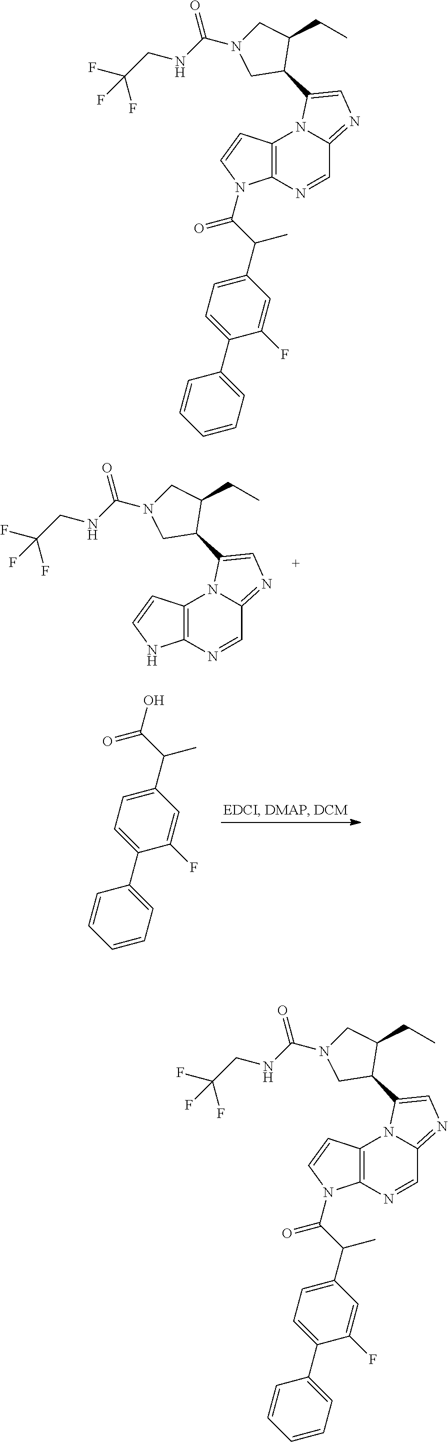 [SMILES: CC[C@@H]1CN(C(=O)NCC(F)(F)F)C[C@@H]1c1cnc2cnc3c(ccn3C(=O)C(C)c3ccc(-c4ccccc4)c(F)c3)n12 ; CC[C@@H]1CN(C(=O)NCC(F)(F)F)C[C@@H]1c1cnc2cnc3[nH]ccc3n12 ; CC(C(=O)O)c1ccc(-c2ccccc2)c(F)c1 ; CC[C@@H]1CN(C(=O)NCC(F)(F)F)C[C@@H]1c1cnc2cnc3c(ccn3C(=O)C(C)c3ccc(-c4ccccc4)c(F)c3)n12]

### Synthesis of (3S, 4R)-3-ethyl-4-(3-(2-(2-fluoro-[1, 1′-biphenyl]-4-yl) propanoyl)-3H-imidazo [1, 2-a] pyrrolo [2, 3-e] pyrazin-8-yl)-N-(2, 2, 2-trifluoroethyl) pyrrolidine-1-carboxamide

(3S, 4R)-3-ethyl-4-(3H-imidazo [1, 2-a] pyrrolo [2, 3-e] pyrazin-8-yl)-N-(2, 2, 2-trifluoroethyl) pyrrolidine-1-carboxamide (Upadacitinib, 100 mg, 0.25 mmol), 4-dimethylamino pyridine (DMAP, 3 mg, 0.025 mmol), 2-(2-fluoro-4-biphenyl) propanoic acid (flurbiprofen, 64 mg, 0.264 mmol) and 1-(3-dimethylaminopropyl)-3-ethylcarbodiimide hydrochloride (EDCI, 73 mg, 0.377 mmol) were dissolved in dichloromethane (4 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=50:1 to 25:1) to give the title compound as a white solid, 0.03 g, 9.9% yield. MS (m/z): [M+H]⁺ calcd for C₃₂H₃₀F₄N₆O₂, 607.24; found, 607.3. ¹H NMR (400 MHz, DMSO-d₆) δ 8.93 (d, J=2.0 Hz, 1H), 8.10 (dd, J=4.2, 2.5 Hz, 1H), 7.65 (d, J=6.9 Hz, 1H), 7.58-7.32 (m, 9H), 6.98 (t, J=5.8 Hz, 1H), 6.09 (p, J=6.9 Hz, 1H), 4.36 (s, 1H), 3.95-3.74 (m, 4H), 3.69 (dt, J=10.7, 6.1 Hz, 1H), 3.26 (dd, J=10.2, 5.3 Hz, 1H), 3.09 (td, J=7.3, 4.7 Hz, 1H), 1.66 (d, J=6.9 Hz, 3H), 1.12-0.96 (m, 1H), 0.93-0.75 (m, 1H), 0.63 (dt, J=10.2, 7.3 Hz, 3H).

### Example 126

### (3R, 4S)-3-(3-(2-(3-benzoylphenyl) propanoyl)-3H-imidazo [1, 2-a] pyrrolo [2, 3-e] pyrazin-8-yl)-4-ethyl-N-(2, 2, 2-trifluoroethyl) pyrrolidine-1-carboxamide

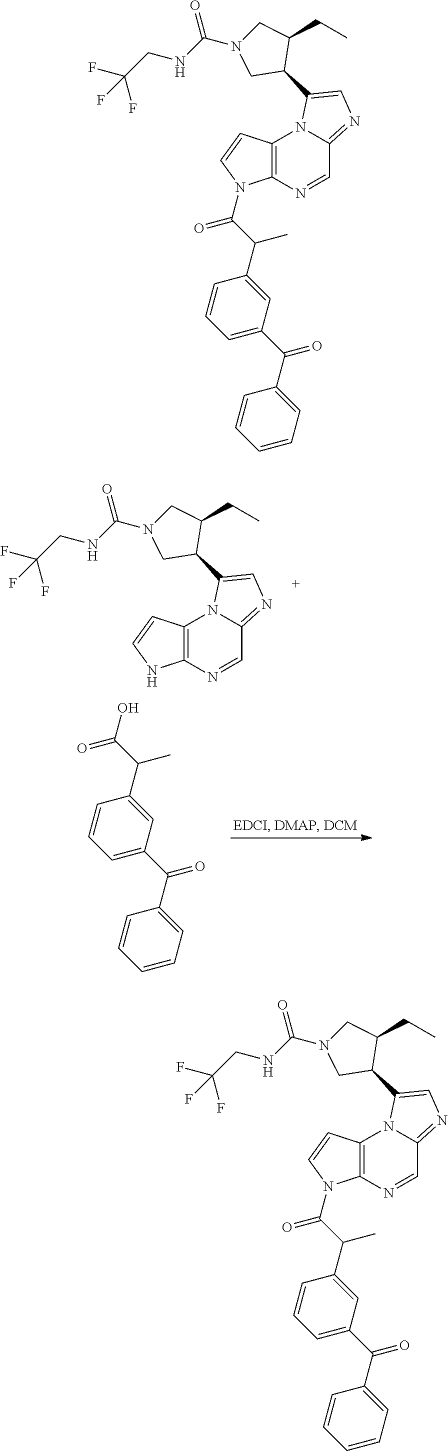 [SMILES: CC[C@@H]1CN(C(=O)NCC(F)(F)F)C[C@@H]1c1cnc2cnc3c(ccn3C(=O)C(C)c3cccc(C(=O)c4ccccc4)c3)n12 ; CC[C@@H]1CN(C(=O)NCC(F)(F)F)C[C@@H]1c1cnc2cnc3[nH]ccc3n12 ; CC(C(=O)O)c1cccc(C(=O)c2ccccc2)c1 ; CC[C@@H]1CN(C(=O)NCC(F)(F)F)C[C@@H]1c1cnc2cnc3c(ccn3C(=O)C(C)c3cccc(C(=O)c4ccccc4)c3)n12]

### Synthesis of (3R, 4S)-3-(3-(2-(3-benzoylphenyl) propanoyl)-3H-imidazo [1, 2-a] pyrrolo [2, 3-e] pyrazin-8-yl)-4-ethyl-N-(2, 2, 2-trifluoroethyl) pyrrolidine-1-carboxamide

(3S, 4R)-3-ethyl-4-(3H-imidazo [1, 2-a] pyrrolo [[2, 3-e] pyrazin-8-yl)-N-(2, 2, 2-trifluoroethyl) pyrrolidine-1-carboxamide (Upadacitinib, 100 mg, 0.25 mmol), 4-dimethylamino pyridine (DMAP, 3 mg, 0.025 mmol), 2-(3-benzoylphenyl) propanoic acid (ketoprofen, 67 mg, 0.264 mmol) and 1-(3-dimethylaminopropyl)-3-ethylcarbodiimide hydrochloride (EDCI, 73 mg, 0.377 mmol) were dissolved in dichloromethane (4 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=50:1 to 25:1) to give the title compound as a white solid, 0.13 g, 84.4% yield. MS (m/z): [M+H]⁺ calcd for C₃₃H₃₁F₃N₆O₃, 617.24; found, 617.2.1H NMR (400 MHz, DMSO-d₆) δ 8.80 (d, J=5.8 Hz, 1H), 8.07 (dd, J=4.3, 1.8 Hz, 1H), 7.85-7.71 (m, 2H), 7.71-7.57 (m, 5H), 7.57-7.47 (m, 3H), 7.45 (d, J=4.3 Hz, 1H), 6.97 (t, J=6.3 Hz, 1H), 6.09 (p, J=7.0 Hz, 1H), 4.35 (q, J=6.5 Hz, 1H), 3.90-3.63 (m, 5H), 3.26 (dt, J=10.1, 5.1 Hz, 1H), 2.58-2.49 (m, 1H), 1.64 (dd, J=7.0, 2.1 Hz, 3H), 1.03 (dp, J=20.6, 7.0 Hz, 1H), 0.81 (s, 1H), 0.61 (dt, J=14.6, 7.3 Hz, 3H).

### Example 127

### (3R, 4S)-3-(3-(2-(2-((2, 6-dichlorophenyl) amino) phenyl) acetyl)-3H-imidazo [1, 2-a] pyrrolo [2, 3-el pyrazin-8-yl)-4-ethyl-N-(2, 2, 2-trifluoroethyl) pyrrolidine-1-carboxamide

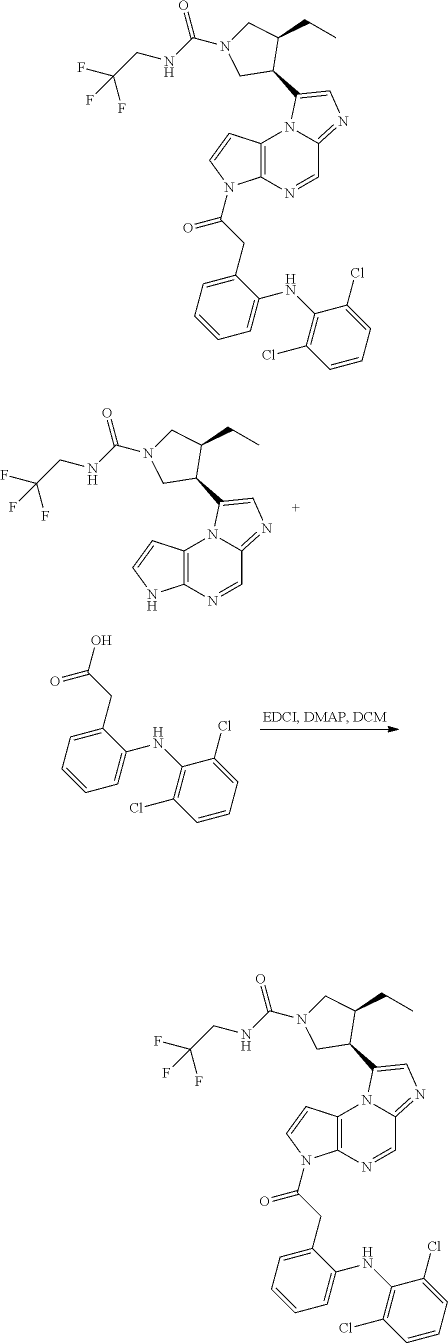 [SMILES: CC[C@@H]1CN(C(=O)NCC(F)(F)F)C[C@@H]1c1cnc2cnc3c(ccn3C(=O)Cc3ccccc3Nc3c(Cl)cccc3Cl)n12 ; CC[C@@H]1CN(C(=O)NCC(F)(F)F)C[C@@H]1c1cnc2cnc3[nH]ccc3n12 ; O=C(O)Cc1ccccc1Nc1c(Cl)cccc1Cl ; CC[C@@H]1CN(C(=O)NCC(F)(F)F)C[C@@H]1c1cnc2cnc3c(ccn3C(=O)Cc3ccccc3Nc3c(Cl)cccc3Cl)n12]

### Synthesis of (3R, 4S)-3-(3-(2-(2-((2, 6-dichlorophenyl) amino) phenyl) acetyl)-3H-imidazo [1, 2-a] pyrrolo [2, 3-e] pyrazin-8-yl)-4-ethyl-N-(2, 2, 2-trifluoroethyl) pyrrolidine-1-carboxamide

(3S, 4R)-3-ethyl-4-(3H-imidazo [1, 2-a] pyrrolo [2, 3-e] pyrazin-8-yl)-N-(2, 2, 2-trifluoroethyl) pyrrolidine-1-carboxamide (Upadacitinib, 100 mg, 0.25 mmol), 4-dimethylamino pyridine (DMAP, 3 mg, 0.025 mmol), 2-(2, 6-dichloroanilino) phenylacetic acid (diclofenac, 78 mg, 0.264 mmol) and 1-(3-dimethylaminopropyl)-3-ethylcarbodiimide hydrochloride (EDCI, 73 mg, 0.377 mmol) were dissolved in dichloromethane (4 mL) and stirred at room temperature for 24 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=50:1 to 25:1) to give the title compound as a white solid, 0.011 g, 6.6% yield. MS (m/z): [M+H]⁺ calcd for C₃₁H₂₈Cl₂F₃N₇O₂, 658.16; found, 658.1.1H NMR (400 MHz, Chloroform-d) δ 8.85 (s, 1H), 8.00 (d, J=4.1 Hz, 1H), 7.58 (s, 1H), 7.38 (dd, J=7.5, 1.5 Hz, 1H), 7.26 (d, J=8.1 Hz, 2H), 7.08 (td, J=7.8, 1.6 Hz, 1H), 6.93-6.85 (m, 4H), 6.52 (d, J=8.0 Hz, 1H), 5.11 (s, 2H), 4.69 (t, J=6.3 Hz, 1H), 4.15 (q, J=6.1 Hz, 1H), 3.89 (tt, J=15.6, 8.6 Hz, 4H), 3.72-3.66 (m, 1H), 3.30 (t, J=8.2 Hz, 1H), 2.57 (dd, J=11.0, 6.3 Hz, 1H), 1.27-1.14 (m, 1H), 0.84-0.77 (m, 1H), 0.69 (t, J=7.3 Hz, 3H).

### Example 128

### (3S, 4R)-3-ethyl-4-(3-(2-(3-phenoxyphenyl) propanoyl)-3H-imidazo [1, 2-a] pyrrolo [2, 3-e] pyrazin-8-yl)-N-(2, 2, 2-trifluoroethyl) pyrrolidine-1-carboxamide

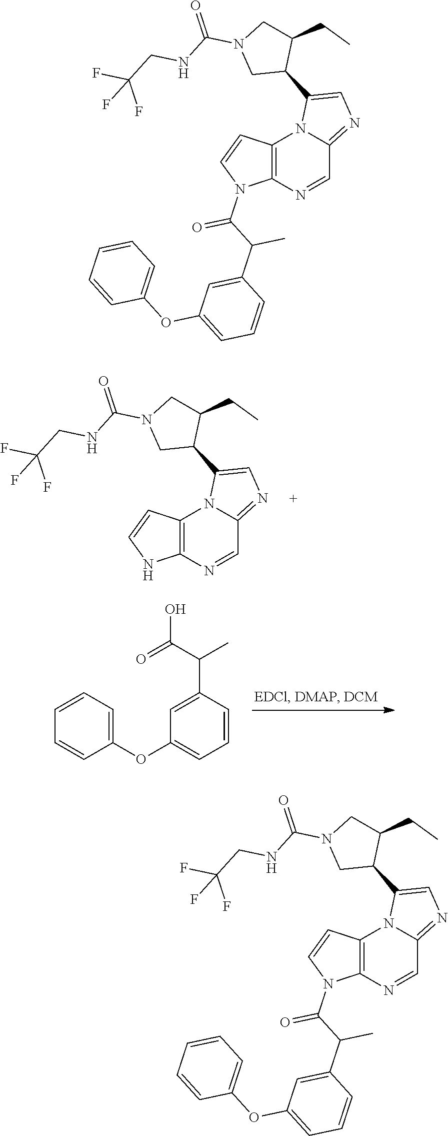 [SMILES: CC[C@@H]1CN(C(=O)NCC(F)(F)F)C[C@@H]1c1cnc2cnc3c(ccn3C(=O)C(C)c3cccc(Oc4ccccc4)c3)n12 ; CC[C@@H]1CN(C(=O)NCC(F)(F)F)C[C@@H]1c1cnc2cnc3[nH]ccc3n12 ; CC(C(=O)O)c1cccc(Oc2ccccc2)c1 ; CC[C@@H]1CN(C(=O)NCC(F)(F)F)C[C@@H]1c1cnc2cnc3c(ccn3C(=O)C(C)c3cccc(Oc4ccccc4)c3)n12]

### Synthesis of (3S, 4R)-3-ethyl-4-(3-(2-(3-phenoxyphenyl) propanoyl)-3H-imidazo [1, 2-a] pyrrolo [2, 3-e] pyrazin-8-yl)-N-(2, 2, 2-trifluoroethyl) pyrrolidine-1-carboxamide

(3S, 4R)-3-ethyl-4-(3H-imidazo [1, 2-a] pyrrolo [2, 3-e] pyrazin-8-yl)-N-(2, 2, 2-trifluoroethyl) pyrrolidine-1-carboxamide (EDCI, 100 mg, 0.25 mmol), 4-dimethylamino pyridine (DMAP, 3 mg, 0.025 mmol), 2-(3-phenoxyphenyl) propanoic acid (fenoprofen, 64 mg, 0.264 mmol) and 1-(3-dimethylaminopropyl)-3-ethylcarbodiimide hydrochloride (Upadacitinib, 73 mg, 0.377 mmol) were dissolved in dichloromethane (4 mL) and stirred at room temperature for 20 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=50:1 to 25:1) to give the title compound as a white solid, 0.101 g, 66.8% yield. MS (m/z): [M+H]⁺ calcd for C₃₂H₃₁F₃N₆O₃, 605.24; found, 605.2.1H NMR (400 MHz, DMSO-d₆) δ 8.74 (d, J=6.8 Hz, 1H), 8.04 (t, J=3.9 Hz, 1H), 7.65 (d, J=6.7 Hz, 1H), 7.49-7.26 (m, 4H), 7.23-6.80 (m, 7H), 5.97 (p, J=6.8 Hz, 1H), 4.38-4.30 (m, 1H), 3.92-3.75 (m, 3H), 3.69 (dd, J=8.4, 6.0 Hz, 2H), 3.26 (dt, J=10.3, 6.7 Hz, 1H), 2.52-2.48 (m, 1H), 1.62-1.55 (m, 3H), 1.07-0.98 (m, 1H), 0.90-0.78 (m, 1H), 0.63 (td, J=7.4, 3.7 Hz, 3H).

### Example 129

### (3R, 4S)-3-(3-(2-((2, 3-dimethylphenyl) amino) benzoyl)-3H-imidazo [1, 2-a] pyrrolo [2, 3-e] pyrazin-8-yl)-4-ethyl-N-(2, 2, 2-trifluoroethyl) pyrrolidine-1-carboxamide

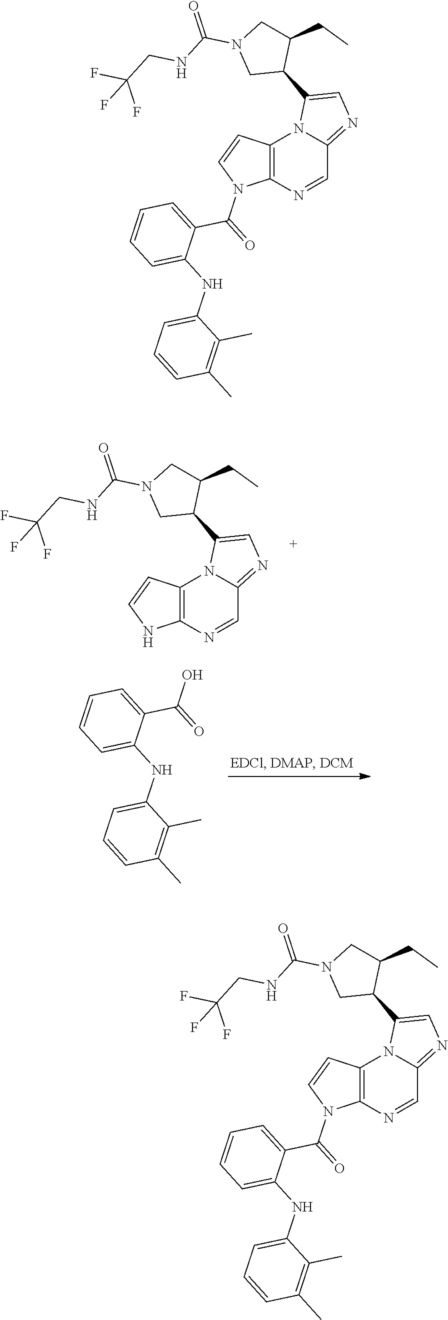

### Synthesis of (3R, 4S)-3-(3-(2-((2, 3-dimethylphenyl) amino) benzoyl)-3H-imidazo [1, 2-a] pyrrolo [2, 3-e] pyrazin-8-yl)-4-ethyl-N-(2, 2, 2-trifluoroethyl) pyrrolidine-1-carboxamide

(3S, 4R)-3-ethyl-4-(3H-imidazo [1, 2-a] pyrrolo [2, 3-e] pyrazin-8-yl)-N-(2, 2, 2-trifluoroethyl) pyrrolidine-1-carboxamide (Upadacitinib, 100 mg, 0.25 mmol), 4-dimethylamino pyridine (DMAP, 3 mg, 0.025 mmol), 2-((2, 3-dimethylphenyl) amino) benzoic acid (mefenamic acid, 64 mg, 0.264 mmol) and 1-(3-dimethylaminopropyl)-3-ethylcarbodiimide hydrochloride (EDCI, 73 mg, 0.377 mmol) were dissolved in dichloromethane (4 mL) and stirred at room temperature for 20 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=50:1 to 25:1) to give the title compound as a yellow solid, 0.012 g, 85.5% yield. MS (m/z): [M+H]⁺ calcd for C₃₂H₃₂F₃N₇O₂, 604.264; found, 604.2. ¹H NMR (400 MHz, DMSO-d₆) δ 8.65 (s, 1H), 8.57 (s, 1H), 7.77 (d, J=3.9 Hz, 1H), 7.61 (s, 1H), 7.48-7.36 (m, 3H), 7.09-6.95 (m, 4H), 6.88-6.74 (m, 2H), 4.38 (t, J=6.5 Hz, 1H), 3.92-3.70 (m, 5H), 3.31-3.25 (m, 1H), 2.63-2.52 (m, 1H), 2.23 (s, 3H), 2.00 (s, 3H), 1.19-1.04 (m, 1H), 0.90-0.82 (m, 1H), 0.68 (t, J=7.3 Hz, 3H).

### Example 130

### (3R, 4S)-3-(3-(2-((3-chloro-2-methylphenyl) amino) benzoyl)-3H-imidazo [1, 2-a] pyrrolo [2, 3-e] pyrazin-8-yl)-4-ethyl-N-(2, 2, 2-trifluoroethyl) pyrrolidine-1-carboxamide

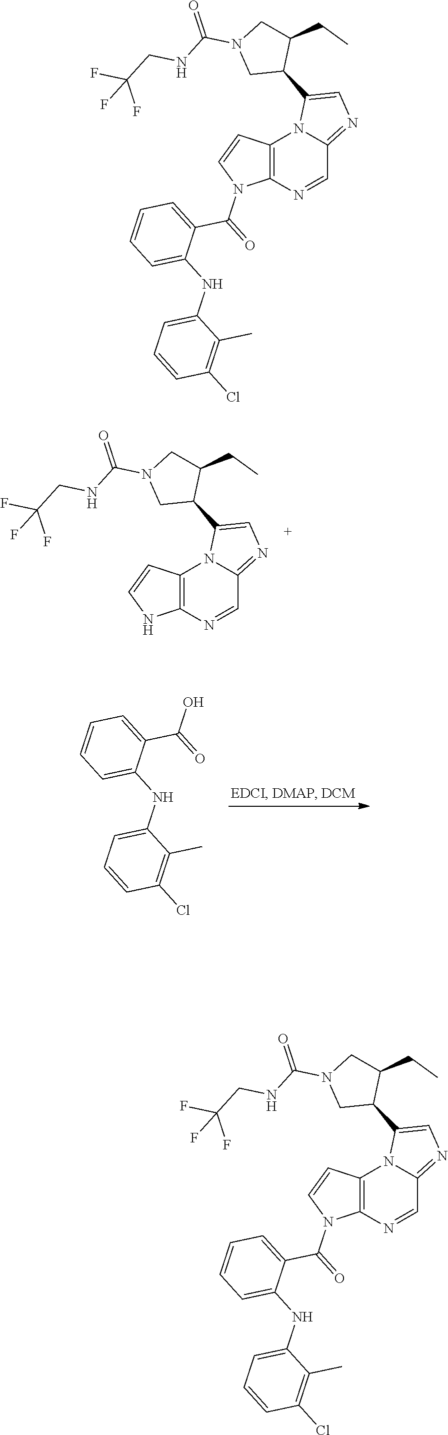 [SMILES: CC[C@@H]1CN(C(=O)NCC(F)(F)F)C[C@@H]1c1cnc2cnc3c(ccn3C(=O)c3ccccc3Nc3cccc(Cl)c3C)n12 ; CC[C@@H]1CN(C(=O)NCC(F)(F)F)C[C@@H]1c1cnc2cnc3[nH]ccc3n12 ; Cc1c(Cl)cccc1Nc1ccccc1C(=O)O ; CC[C@@H]1CN(C(=O)NCC(F)(F)F)C[C@@H]1c1cnc2cnc3c(ccn3C(=O)c3ccccc3Nc3cccc(Cl)c3C)n12]

### Synthesis of (3R, 4S)-3-(3-(2-((3-chloro-2-methylphenyl) amino) benzoyl)-3H-imidazo [1, 2-a] pyrrolo [2, 3-e] pyrazin-8-yl)-4-ethyl-N-(2, 2, 2-trifluoroethyl) pyrrolidine-1-carboxamide

(3S, 4R)-3-ethyl-4-(3H-imidazo [1, 2-a] pyrrolo [2, 3-e] pyrazin-8-yl)-N-(2, 2, 2-trifluoroethyl) pyrrolidine-1-carboxamide (Upadacitinib, 100 mg, 0.25 mmol), 4-dimethylamino pyridine (DMAP, 3 mg, 0.025 mmol), 2-((3-chloro-2-methylphenyl) amino) benzoic acid (tolfenamic acid, 68 mg, 0.264 mmol) and 1-(3-dimethylaminopropyl)-3-ethylcarbodiimide hydrochloride (EDCI, 73 mg, 0.377 mmol) were dissolved in dichloromethane (4 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=50:1 to 25:1) to give the title compound as a yellow solid, 0.09 g, 29.7% yield. MS (m/z): [M+H]⁺ calcd for C₃₁H₂₉ClF₃N₇O₂, 624.20; found, 624.2. ¹H NMR (400 MHz, DMSO-d₆) δ 8.61 (s, 1H), 8.25 (s, 1H), 7.67-7.58 (m, 1H), 7.56-7.44 (m, 2H), 7.36 (d, J=4.2 Hz, 1H), 7.22 (d, J=4.9 Hz, 1H), 7.10-6.85 (m, 5H), 6.81-6.75 (m, 1H), 4.36 (d, J=6.6 Hz, 1H), 3.96-3.65 (m, 5H), 3.34-3.21 (m, 1H), 2.62-2.53 (m, 1H), 1.96 (s, 3H), 1.16-1.01 (m, 1H), 0.93-0.76 (m, 1H), 0.72-0.66 (m, 3H).

### Example 131

### (3S, 4R)-3-ethyl-4-(3-((S)-2-(6-) methoxynaphthalen-2-yl) propanoyl)-3H-imidazo [1, 2-a] pyrrolo [2, 3-e] pyrazin-8-yl)-N-(2, 2, 2-trifluoroethyl) pyrrolidine-1-carboxamide

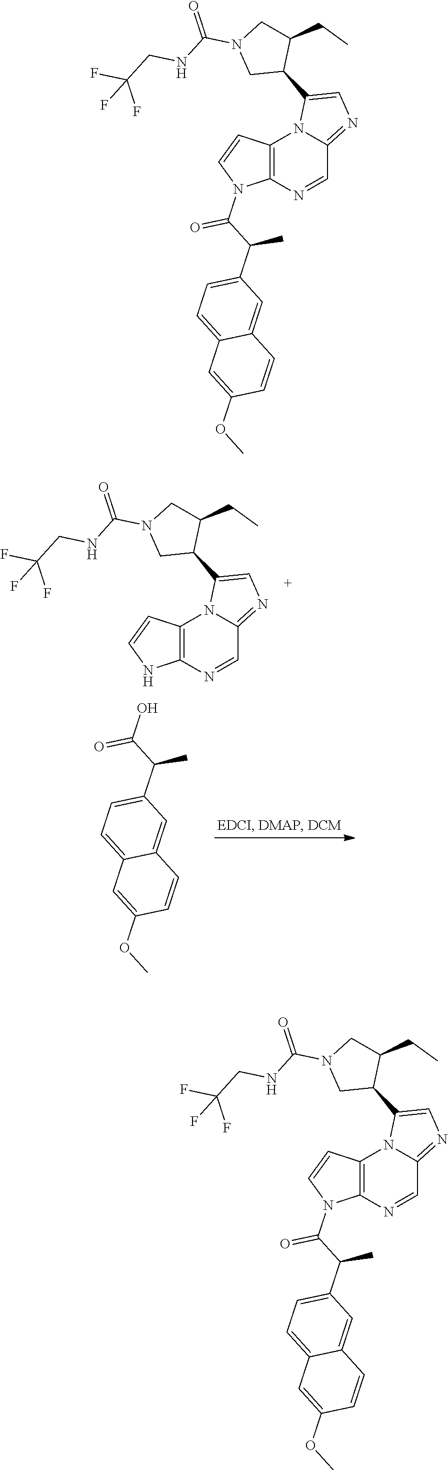

### Synthesis of (3S, 4R)-3-ethyl-4-(3-((S)-2-(6-) methoxynaphthalen-2-yl) propanoyl)-3H-imidazo [1, 2-a] pyrrolo [2, 3-e] pyrazin-8-yl)-N-(2, 2, 2-trifluoroethyl) pyrrolidine-1-carboxamide

(3S, 4R)-3-ethyl-4-(3H-imidazo [1, 2-a] pyrrolo [2, 3-e] pyrazin-8-yl)-N-(2, 2, 2-trifluoroethyl) pyrrolidine-1-carboxamide (Upadacitinib, 100 mg, 0.25 mmol), 4-dimethylamino pyridine (DMAP, 3 mg, 0.025 mmol), (S)-(+)-2-(6-methoxy-2-naphthyl) propanoic acid (Naproxen, 60 mg, 0.264 mmol) and 1-(3-dimethylaminopropyl)-3-ethylcarbodiimide hydrochloride (EDCI, 73 mg, 0.377 mmol) were dissolved in dichloromethane (4 mL) and stirred at room temperature for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=50:1 to 25:1) to give the title compound as a white solid, 0.028 g, 18.9% yield. MS (m/z): [M+H]⁺ calcd for C₃₁H₃₁F₃N₆O₃, 593.24; found, 593.2. ¹H NMR (400 MHz, DMSO-d₆) δ 8.92 (s, 1H), 8.08 (d, J=4.2 Hz, 1H), 7.91-7.68 (m, 3H), 7.63 (s, 1H), 7.57 (dd, J=8.5, 1.8 Hz, 1H), 7.43-7.39 (m, 1H), 7.25 (d, J=2.6 Hz, 1H), 7.12 (dd, J=9.0, 2.6 Hz, 1H), 6.96 (t, J=6.2 Hz, 1H), 6.13 (q, J=6.9 Hz, 1H), 4.33 (d, J=6.7 Hz, 1H), 3.97-3.72 (m, 7H), 3.65 (dd, J=10.3, 6.7 Hz, 1H), 3.24 (dd, J=10.3, 5.7 Hz, 1H), 2.53-2.42 (m, 1H), 1.68 (d, J=6.9 Hz, 3H), 1.03-0.92 (m, 1H), 0.89-0.71 (m, 1H), 0.57 (t, J=7.3 Hz, 3H).

### Example 132

### (3S, 4R)-3-ethyl-4-(3-(2-(4-((2-oxocyclopentyl) methyl) phenyl) propanoyl)-3H-imidazo [1, 2-a] pyrrolo [2, 3-e] pyrazin-8-yl)-N-(2, 2, 2-trifluoroethyl) pyrrolidine-1-carboxamide

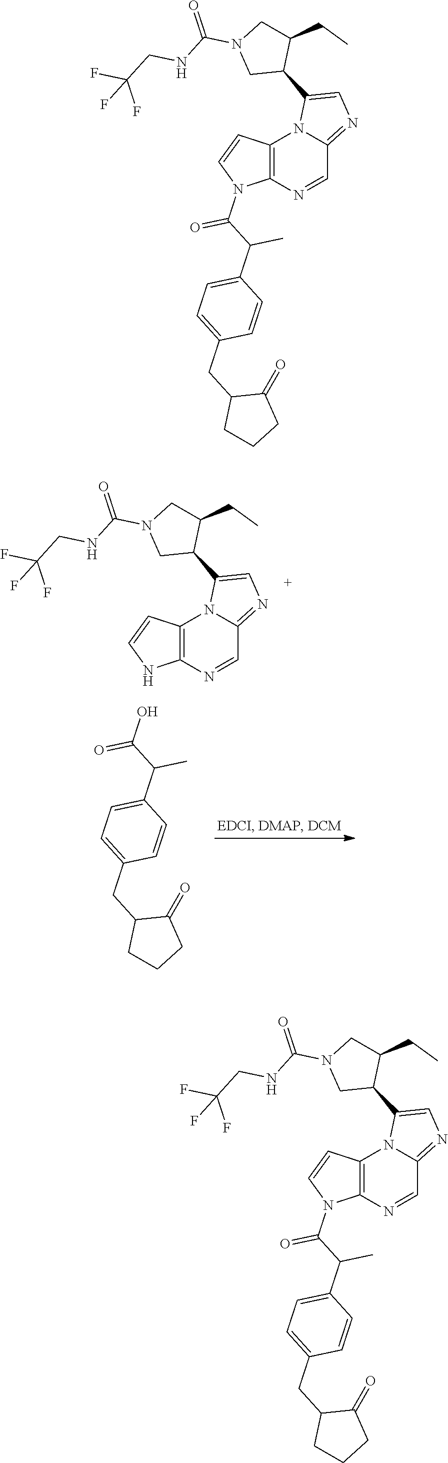

### Synthesis of (3S, 4R)-3-ethyl-4-(3-(2-(4-((2-oxocyclopentyl) methyl) phenyl) propanoyl)-3H-imidazo [1, 2-a] pyrrolo [2, 3-e] pyrazin-8-yl)-N-(2, 2, 2-trifluoroethyl) pyrrolidine-1-carboxamide

(3S, 4R)-3-ethyl-4-(3H-imidazo [1, 2-a] pyrrolo [2, 3-e] pyrazin-8-yl)-N-(2, 2, 2-trifluoroethyl) pyrrolidine-1-carboxamide (Upadacitinib, 100 mg, 0.25 mmol), 4-dimethylamino pyridine (DMAP, 3 mg, 0.025 mmol), 2-[4-(2-oxocyclopentan-1-ylmethyl) phenyl] propanoic acid (loxoprofen, 65 mg, 0.264 mmol) and 1-(3-dimethylaminopropyl)-3-ethylcarbodiimide hydrochloride (EDCI, 73 mg, 0.377 mmol) were dissolved in dichloromethane (4 mL) and stirred at room temperature for 20 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=50:1 to 25:1) to give the title compound as a white solid, 0.066 g, 43.4% yield. MS (m/z): [M+H]⁺ calcd for C₃₂H₃₅F₃N₆O₃, 609.27, found 609.3. ¹H NMR (400 MHz, DMSO-d₆) δ 8.88 (d, J=0.9 Hz, 1H), 8.04 (t, J=3.9 Hz, 1H), 7.63 (d, J=7.0 Hz, 1H), 7.44-7.30 (m, 3H), 7.22-7.07 (m, 2H), 6.97 (t, J=6.3 Hz, 1H), 6.00 (p, J=7.0 Hz, 1H), 4.34 (d, J=6.2 Hz, 1H), 3.96-3.72 (m, 4H), 3.67 (ddd, J=10.9, 6.7, 4.6 Hz, 1H), 3.25 (dt, J=10.3, 5.1 Hz, 1H), 2.93-2.84 (m, 1H), 2.56-2.51 (m, 1H), 2.43-2.26 (m, 2H), 2.20 (dd, J=18.6, 8.4 Hz, 1H), 2.12-1.96 (m, 1H), 1.91-1.73 (m, 2H), 1.72-1.50 (m, 4H), 1.48-1.36 (m, 1H), 1.02 (ddq, J=19.7, 12.8, 7.1, 6.6 Hz, 1H), 0.80 (ddt, J=16.9, 13.8, 6.5 Hz, 1H), 0.62 (dt, J=10.3, 7.3 Hz, 3H).

### Example 133

### (3R, 4S)-3-(3-(2-(1-(4-Chlorobenzoyl)-5-methoxy-2-methyl-1H-indol-3-yl) acetyl)-3H-imidazo [1, 2-a] pyrrolo [2, 3-e] pyrazin-8-yl)-4-ethyl-N-(2, 2, 2-trifluoroethyl) pyrrolidine-1-carboxamide

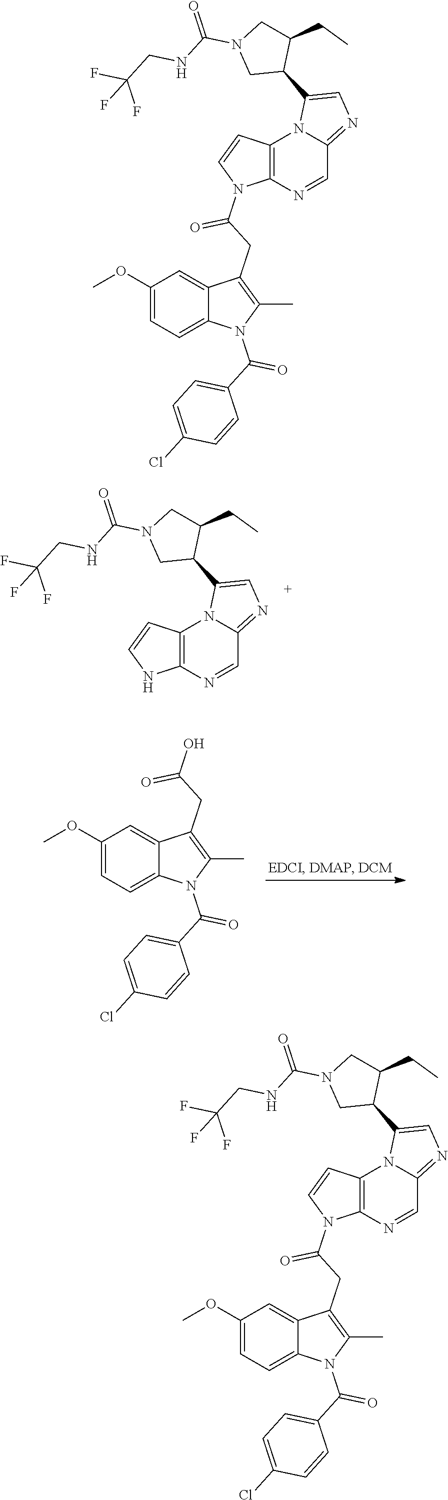

### Synthesis of (3R, 4S)-3-(3-(2-(1-(4-Chlorobenzoyl)-5-methoxy-2-methyl-1H-indol-3-yl) acetyl)-3H-imidazo [1, 2-a] pyrrolo [2, 3-e] pyrazin-8-yl)-4-ethyl-N-(2, 2, 2-trifluoroethyl) pyrrolidine-1-carboxamide

(3S, 4R)-3-ethyl-4-(3H-imidazo [1, 2-a] pyrrolo [2, 3-e] pyrazin-8-yl)-N-(2, 2, 2-trifluoroethyl) pyrrolidine-1-carboxamide (Upadacitinib, 100 mg, 0.25 mmol), 4-dimethylamino pyridine (DMAP, 3 mg, 0.025 mmol), 1-(4-chlorobenzoyl)-5-methoxy-2-methyl-1H-indole-3-acetic acid (indomethacin, 95 mg, 0.264 mmol) and 1-(3-dimethylaminopropyl)-3-ethylcarbodiimide hydrochloride (EDCI, 73 mg, 0.377 mmol) were dissolved in dichloromethane (4 mL) and stirred at room temperature for 20 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=50:1 to 25:1) to give the title compound as a light yellow solid, 0.139 g, 77.3% yield. MS (m/z): [M+H]⁺ calcd for C₃₆H₃₃ClF₃N₇O₄, 720.22, found 720.1. ¹H NMR (400 MHz, DMSO-d₆) δ 8.95 (s, 1H), 8.10 (d, J=4.1 Hz, 1H), 7.75-7.63 (m, 5H), 7.50 (d, J=4.2 Hz, 1H), 7.18 (d, J=2.6 Hz, 1H), 7.00 (dd, J=7.7, 5.0 Hz, 2H), 6.73 (dd, J=9.0, 2.5 Hz, 1H), 5.06 (d, J=4.3 Hz, 2H), 4.41 (q, J=6.4 Hz, 1H), 3.93-3.83 (m, 3H), 3.83-3.67 (m, 5H), 3.27 (dd, J=10.2, 5.9 Hz, 1H), 2.58 (s, 1H), 2.27 (s, 3H), 1.07 (ddd, J=12.8, 7.4, 4.9 Hz, 1H), 0.82 (ddd, J=13.3, 10.0, 7.0 Hz, 1H), 0.65 (t, J=7.3 Hz, 3H).

### Example 134

### (3R, 4S)-3-(3-(2-(4-acetamidophenyl) acetyl)-3H-imidazo [1, 2-a] pyrrolo [2, 3-e] pyrazin-8-yl)-4-ethyl-N-(2, 2, 2-trifluoroethyl) pyrrolidine-1-carboxamide

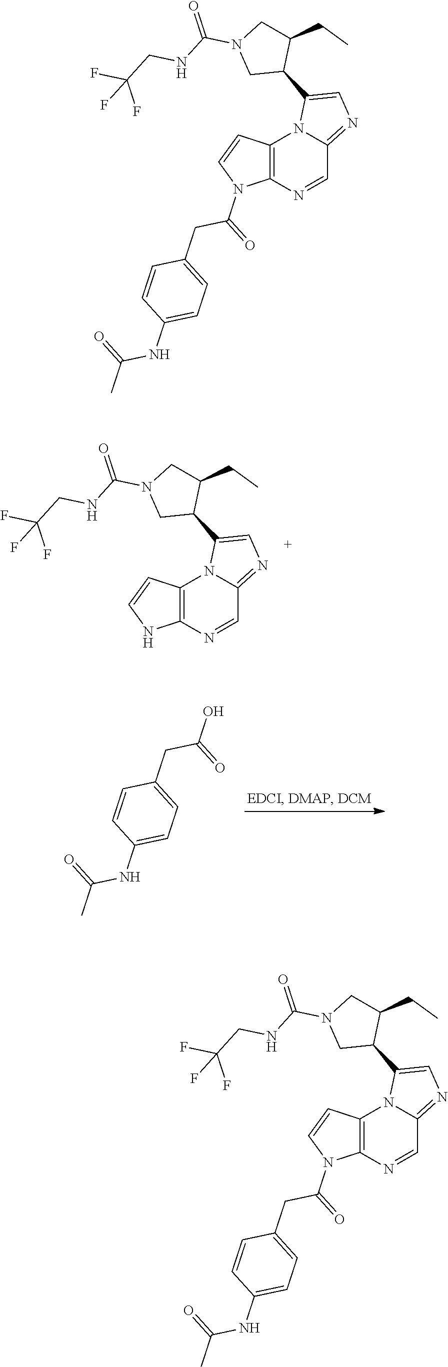 [SMILES: CC[C@@H]1CN(C(=O)NCC(F)(F)F)C[C@@H]1c1cnc2cnc3c(ccn3C(=O)Cc3ccc(NC(C)=O)cc3)n12 ; CC[C@@H]1CN(C(=O)NCC(F)(F)F)C[C@@H]1c1cnc2cnc3[nH]ccc3n12 ; CC(=O)Nc1ccc(CC(=O)O)cc1 ; CC[C@@H]1CN(C(=O)NCC(F)(F)F)C[C@@H]1c1cnc2cnc3c(ccn3C(=O)Cc3ccc(NC(C)=O)cc3)n12]

### Synthesis of (3R, 4S)-3-(3-(2-(4-acetamidophenyl) acetyl)-3H-imidazo [1, 2-a] pyrrolo [2, 3-e] pyrazin-8-yl)-4-ethyl-N-(2, 2, 2-trifluoroethyl) pyrrolidine-1-carboxamide

(3S, 4R)-3-ethyl-4-(3H-imidazo [1, 2-a] pyrrolo [2, 3-e] pyrazin-8-yl)-N-(2, 2, 2-trifluoroethyl) pyrrolidine-1-carboxamide (Upadacitinib, 100 mg, 0.25 mmol), 4-dimethylamino pyridine (DMAP, 3 mg, 0.025 mmol), 2-(4-acetamidophenyl) acetic acid (actarit, 51 mg, 0.264 mmol) and 1-(3-dimethylaminopropyl)-3-ethylcarbodiimide hydrochloride (EDCI, 73 mg, 0.377 mmol) were dissolved in dichloromethane (4 mL) and stirred at room temperature for 20 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=50:1 to 25:1) to give the title compound as a white solid, 0.082 g, 59% yield. MS (m/z): [M+H]⁺ calcd for C₂₇H₂₈F₃N₇O₃, 556.22, found 556.1. ¹H NMR (400 MHz, DMSO-d₆) δ 9.93 (s, 1H), 8.89 (s, 1H), 8.06 (d, J=4.2 Hz, 1H), 7.65 (s, 1H), 7.57-7.48 (m, 2H), 7.46 (d, J=4.1 Hz, 1H), 7.35-7.27 (m, 2H), 6.99 (t, J=6.4 Hz, 1H), 4.88 (d, J=3.6 Hz, 2H), 4.39 (q, J=6.7 Hz, 1H), 3.92-3.74 (m, 4H), 3.70 (dd, J=10.2, 6.7 Hz, 1H), 3.26 (dd, J=10.2, 5.9 Hz, 1H), 2.56 (s, 1H), 2.03 (s, 3H), 1.06 (ddd, J=12.9, 7.7, 5.2 Hz, 1H), 0.89-0.73 (m, 1H), 0.64 (t, J=7.3 Hz, 3H).

### Example 135

(3R, 4S)-3-(3-(2-(1, 8-diethyl-1, 3, 4, 9-tetrahydropyrano [3, 4-b] indol-1-yl) acetyl)-3H-imidazo [1, 2-a] pyrrolo [2, 3-e] pyrazin-8-yl)-4-ethyl-N-(2, 2, 2-trifluoroethyl) pyrrolidine-1-carboxamide

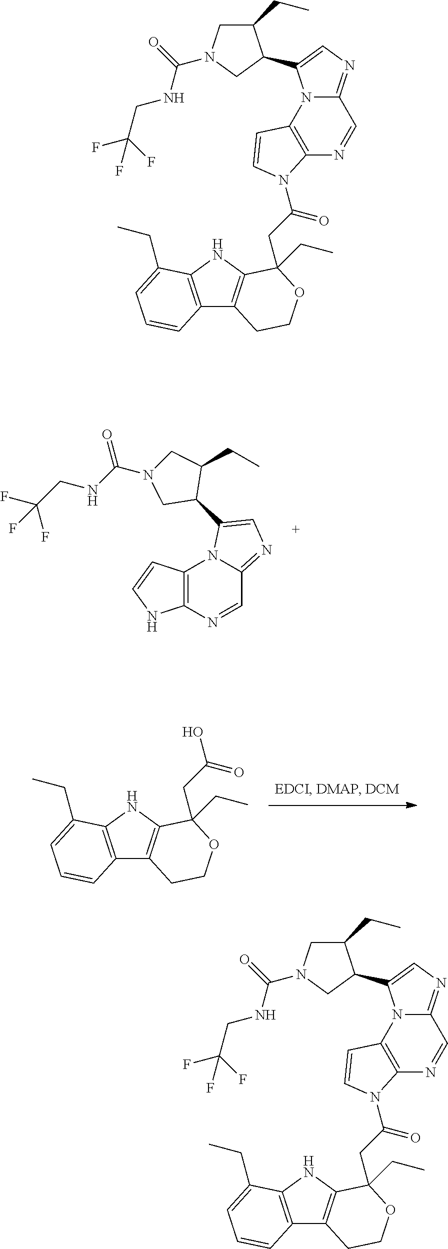 [SMILES: CCc1cccc2c3c([nH]c12)C(CC)(CC(=O)n1ccc2c1ncc1ncc([C@H]4CN(C(=O)NCC(F)(F)F)C[C@H]4CC)n12)OCC3 ; CC[C@@H]1CN(C(=O)NCC(F)(F)F)C[C@@H]1c1cnc2cnc3[nH]ccc3n12 ; CCc1cccc2c3c([nH]c12)C(CC)(CC(=O)O)OCC3 ; CCc1cccc2c3c([nH]c12)C(CC)(CC(=O)n1ccc2c1ncc1ncc([C@H]4CN(C(=O)NCC(F)(F)F)C[C@H]4CC)n12)OCC3]

### Synthesis of (3R, 4S)-3-(3-(2-(1, 8-diethyl-1, 3, 4, 9-tetrahydropyrano [3, 4-b] indol-1-yl) acetyl)-3H-imidazo [1, 2-a] pyrrolo [2, 3-e] pyrazin-8-yl)-4-ethyl-N-(2, 2, 2-trifluoroethyl) pyrrolidine-1-carboxamide

(3S, 4R)-3-ethyl-4-(3H-imidazo [1, 2-a] pyrrolo [2, 3-e] pyrazin-8-yl)-N-(2, 2, 2-trifluoroethyl) pyrrolidine-1-carboxamide (Upadacitinib, 100 mg, 0.25 mmol), 4-dimethylamino pyridine (DMAP, 3 mg, 0.025 mmol), 1, 8-diethyl-1, 3, 4, 9-tetrahydropyran [3, 4-b] indole-1-acetic acid (etodolac, 76 mg, 0.264 mmol) and 1-(3-dimethylaminopropyl)-3-ethylcarbodiimide hydrochloride (EDCI, 73 mg, 0.377 mmol) were dissolved in dichloromethane (4 mL) and stirred at room temperature for 20 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=50:1 to 25:1) to give the title compound as a white solid, 0.108 g, 66.5% yield. MS (m/z): [M+H]⁺ calcd for C₃₄H₃₈F₃N₇O₃, 650.30, found 650.3. ¹H NMR (400 MHz, DMSO-d₆) δ 10.55 (d, J=10.5 Hz, 1H), 8.79 (s, 1H), 8.01 (t, J=4.1 Hz, 1H), 7.62 (d, J=2.5 Hz, 1H), 7.40 (d, J=4.2 Hz, 1H), 7.22 (td, J=7.5, 1.6 Hz, 1H), 6.99 (t, J=6.3 Hz, 1H), 6.89 (td, J=7.4, 5.1 Hz, 2H), 4.61 (dd, J=47.7, 14.4 Hz, 1H), 4.36 (d, J=6.7 Hz, 1H), 4.13 (dd, J=41.9, 14.4 Hz, 1H), 3.95-3.74 (m, 6H), 3.69 (dd, J=10.3, 6.9 Hz, 1H), 3.26 (dd, J=10.3, 5.8 Hz, 1H), 2.90-2.81 (m, 2H), 2.60 (q, J=4.7 Hz, 2H), 2.24-2.14 (m, 2H), 1.25 (td, J=7.4, 2.7 Hz, 4H), 1.10-1.00 (m, 1H), 0.82 (dtd, J=13.7, 7.0, 3.4 Hz, 1H), 0.71 (q, J=7.1 Hz, 3H), 0.64 (td, J=7.3, 2.8 Hz, 3H).

### Example 136

### 2-(8-((3R, 4S)-4-ethyl-1-((2, 2, 2-trifluoroethyl) carbamoyl) pyrrolidin-3-yl)-3H-imidazo [1, 2-a] pyrrolo [2, 3-e] pyrazine-3-carbonyl) phenylacetate

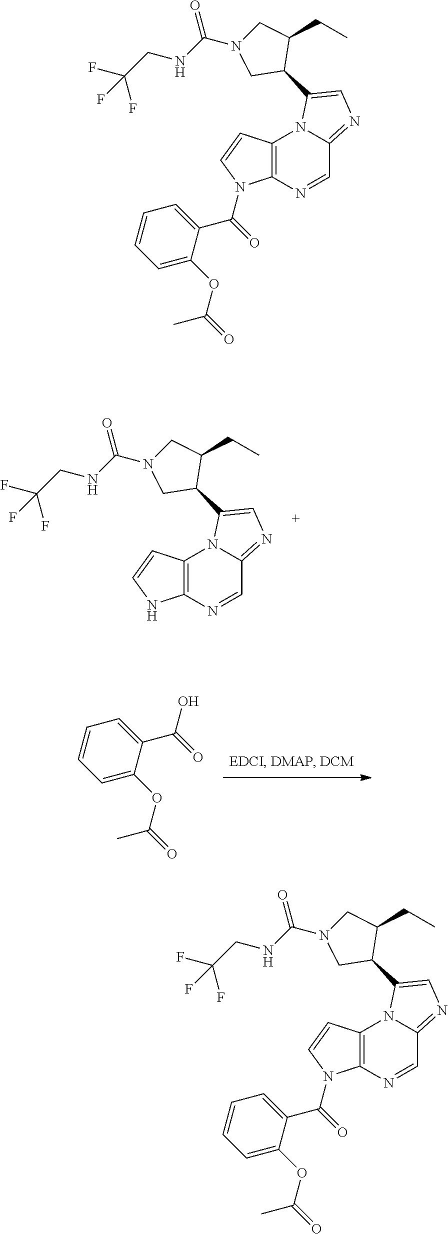 [SMILES: CC[C@@H]1CN(C(=O)NCC(F)(F)F)C[C@@H]1c1cnc2cnc3c(ccn3C(=O)c3ccccc3OC(C)=O)n12 ; CC[C@@H]1CN(C(=O)NCC(F)(F)F)C[C@@H]1c1cnc2cnc3[nH]ccc3n12 ; CC(=O)Oc1ccccc1C(=O)O ; CC[C@@H]1CN(C(=O)NCC(F)(F)F)C[C@@H]1c1cnc2cnc3c(ccn3C(=O)c3ccccc3OC(C)=O)n12]

### Synthesis of 2-(8-((3R, 4S)-4-ethyl-1-((2, 2, 2-trifluoroethyl) carbamoyl) pyrrolidin-3-yl)-3H-imidazo [1, 2-a] pyrrolo [2, 3-e] pyrazine-3-carbonyl) phenylacetate

(3S, 4R)-3-ethyl-4-(3H-imidazo [1, 2-a] pyrrolo [2, 3-e] pyrazin-8-yl)-N-(2, 2, 2-trifluoroethyl) pyrrolidine-1-carboxamide (EDCI, 100 mg, 0.25 mmol), 4-dimethylamino pyridine (DMAP, 3 mg, 0.025 mmol), 2-acetoxy benzoic acid (aspirin, 47.5 mg, 0.264 mmol) and 1-(3-dimethylaminopropyl)-3-ethylcarbodiimide hydrochloride (Upadacitinib, 73 mg, 0.377 mmol) was dissolved in dichloromethane (4 mL) and stirred at room temperature for 20 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=50:1 to 25:1) to give the title compound as a white solid, 0.025 g, 18.4% yield. MS (m/z): [M+H]⁺ calcd for C₂₆H₂₅F₃N₆O₄, 543.19, found 543.1. ¹H NMR (400 MHz, DMSO-d₆) δ 8.56 (s, 1H), 7.85 (d, J=4.2 Hz, 1H), 7.78-7.71 (m, 2H), 7.60 (s, 1H), 7.51-7.44 (m, 2H), 7.34 (dd, J=8.1, 1.0 Hz, 1H), 6.99 (t, J=6.3 Hz, 1H), 4.43-4.35 (m, 1H), 3.92-3.75 (m, 4H), 3.70 (dd, J=10.2, 6.9 Hz, 1H), 3.27-3.20 (m, 1H), 2.62-2.49 (m, 1H), 1.89 (s, 3H), 1.08 (ddd, J=12.9, 7.4, 4.9 Hz, 1H), 0.83-0.75 (m, 1H), 0.64 (t, J=7.3 Hz, 3H).

### Example 137

### Tert-butyl ((S)-2-(8-((3R, 4S)-4-ethyl-1-((2, 2, 2-trifluoroethyl) carbamoyl) pyrrolidin-3-yl)-3H-imidazo [1, 2-a] pyrrolo [2, 3-e] pyrazin-3-yl)-2-oxo-1-phenethyl) carbamate

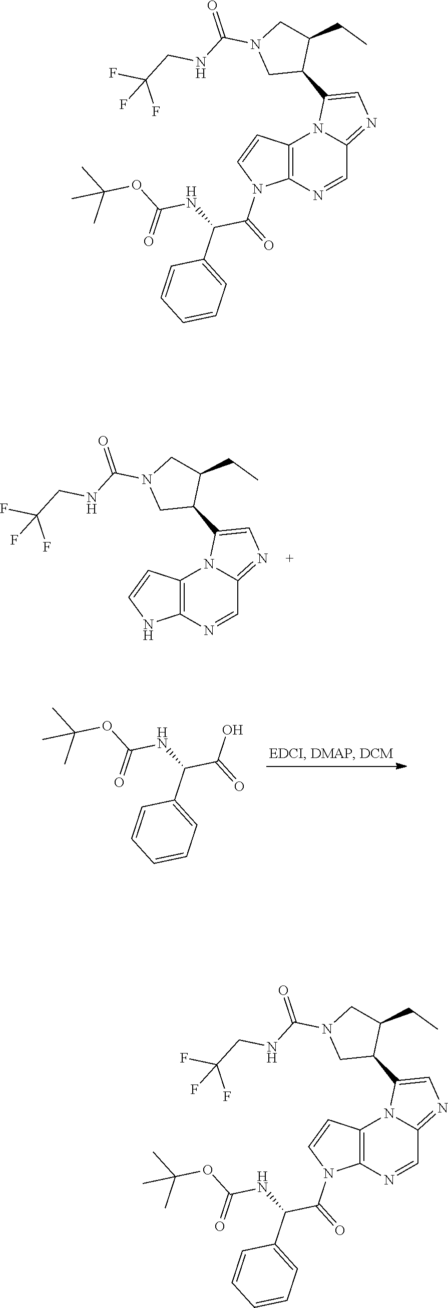 [SMILES: CC[C@@H]1CN(C(=O)NCC(F)(F)F)C[C@@H]1c1cnc2cnc3c(ccn3C(=O)[C@@H](NC(=O)OC(C)(C)C)c3ccccc3)n12 ; CC[C@@H]1CN(C(=O)NCC(F)(F)F)C[C@@H]1c1cnc2cnc3[nH]ccc3n12 ; CC(C)(C)OC(=O)N[C@H](C(=O)O)c1ccccc1 ; CC[C@@H]1CN(C(=O)NCC(F)(F)F)C[C@@H]1c1cnc2cnc3c(ccn3C(=O)[C@@H](NC(=O)OC(C)(C)C)c3ccccc3)n12]

### Synthesis of tert-butyl ((S)-2-(8-((3R, 4S)-4-ethyl-1-((2, 2, 2-trifluoroethyl) carbamoyl) pyrrolidin-3-yl)-3H-imidazo [1, 2-a] pyrrolo [2, 3-e] pyrazin-3-yl)-2-oxo-1-phenethyl) carbamate

(3S, 4R)-3-ethyl-4-(3H-imidazo [1, 2-a] pyrrolo [2, 3-e] pyrazin-8-yl)-N-(2, 2, 2-trifluoroethyl) pyrrolidine-1-carboxamide (Upadacitinib, 100 mg, 0.25 mmol), 4-dimethylamino pyridine (DMAP, 3 mg, 0.025 mmol), N-Boc-L-phenylglycine (66 mg, 0.264 mmol) and 1-(3-dimethylaminopropyl)-3-ethylcarbodiimide hydrochloride (EDCI, 73 mg, 0.377 mmol) were dissolved in dichloromethane (4 mL) and stirred at room temperature for 20 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=50:1 to 25:1) to give the title compound as a white solid, 0.088 g, 57.4% yield. MS (m/z): [M+H]⁺ calcd for C₃₀H₃₄F₃N₇O₄, 614.26, found 614.2. ¹H NMR (400 MHz, DMSO-d₆) δ 8.89 (s, 1H), 8.07 (t, J=4.2 Hz, 1H), 7.99 (dd, J=7.5, 4.5 Hz, 1H), 7.67-7.24 (m, 8H), 6.97 (td, J=6.3, 2.3 Hz, 1H), 4.37-4.29 (m, 1H), 3.91-3.72 (m, 4H), 3.67 (dt, J=10.3, 6.4 Hz, 1H), 3.28-3.18 (m, 1H), 2.52-2.43 (m, 1H), 1.40 (d, J=2.5 Hz, 9H), 1.05-0.92 (m, 1H), 0.88-0.68 (m, 1H), 0.62 (dt, J=14.5, 7.3 Hz, 3H).

### Example 138

### (3S, 4R)-3-ethyl-4-(3-(2-(4-(1-) oxoisoindolin-2-yl) phenyl) butanoyl)-3H-imidazo [1, 2-a] pyrrolo [2, 3-e] pyrazin-8-yl)-N-(2, 2, 2-trifluoroethyl) pyrrolidine-1-carboxamide

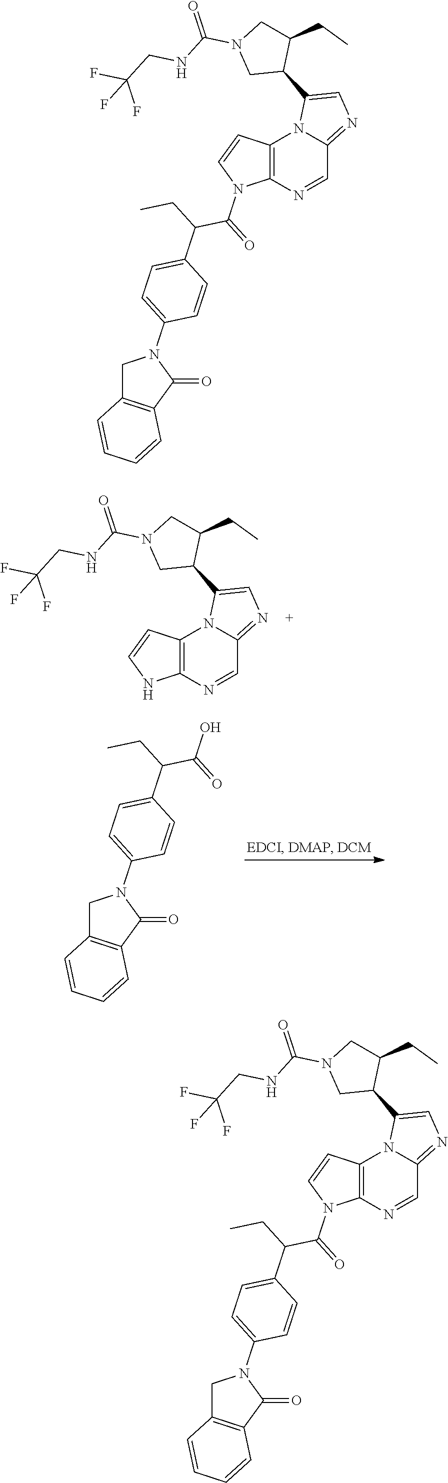

### Synthesis of (3S, 4R)-3-ethyl-4-(3-(2-(4-(1-) oxoisoindolin-2-yl) phenyl) butanoyl)-3H-imidazo [1, 2-a] pyrrolo [2, 3-e] pyrazin-8-yl)-N-(2, 2, 2-trifluoroethyl) pyrrolidine-1-carboxamide

(3S, 4R)-3-ethyl-4-(3H-imidazo [1, 2-a] pyrrolo [2, 3-e] pyrazin-8-yl)-N-(2, 2, 2-trifluoroethyl) pyrrolidine-1-carboxamide (Upadacitinib, 100 mg, 0.25 mmol), 4-dimethylamino pyridine (DMAP, 3 mg, 0.025 mmol), 2-(4-(1-oxoisoindolin-2-yl) phenyl) butanoic acid (indobufen, 78 mg, 0.264 mmol) and 1-(3-dimethylaminopropyl)-3-ethylcarbodiimide hydrochloride (EDCI, 73 mg, 0.377 mmol) were dissolved in dichloromethane (4 mL) and stirred at room temperature for 20 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=50:1 to 25:1) to give the title compound as a white solid, 0.145 g, 88.2% yield. MS (m/z): [M+H]⁺ calcd for C₃₅H₃₄F₃N₇O₃, 658.27, found 658.2. ¹H NMR (400 MHz, DMSO-d₆) δ 8.93 (d, J=1.8 Hz, 1H), 8.07 (dd, J=7.3, 4.1 Hz, 1H), 7.91-7.80 (m, 2H), 7.75 (dd, J=7.6, 3.0 Hz, 1H), 7.71-7.59 (m, 3H), 7.58-7.46 (m, 3H), 7.41 (t, J=4.7 Hz, 1H), 6.96 (td, J=6.3, 2.1 Hz, 1H), 5.86 (t, J=7.4 Hz, 1H), 4.96 (d, J=6.1 Hz, 2H), 4.37-4.29 (m, 1H), 3.91-3.73 (m, 4H), 3.67 (dt, J=10.2, 6.4 Hz, 1H), 3.25 (dd, J=10.3, 5.8 Hz, 1H), 2.56-2.49 (m, 1H), 2.34-2.19 (m, 1H), 1.94 (dtd, J=12.8, 7.5, 4.7 Hz, 1H), 1.12-0.99 (m, 1H), 0.95 (td, J=7.3, 5.7 Hz, 3H), 0.80 (ddd, J=16.9, 14.2, 7.4 Hz, 1H), 0.62 (dt, J=17.9, 7.3 Hz, 3H).

### Example 139

### 3-((3S, 4R)-6-(7-(2-(4-isobutylphenyl) propanoyl)-7H-pyrrolo [2, 3-d] pyrimidin-4-yl)-3-methyl-1, 6-diazaspiro [3.4] oct-1-yl)-3-oxopropanenitrile

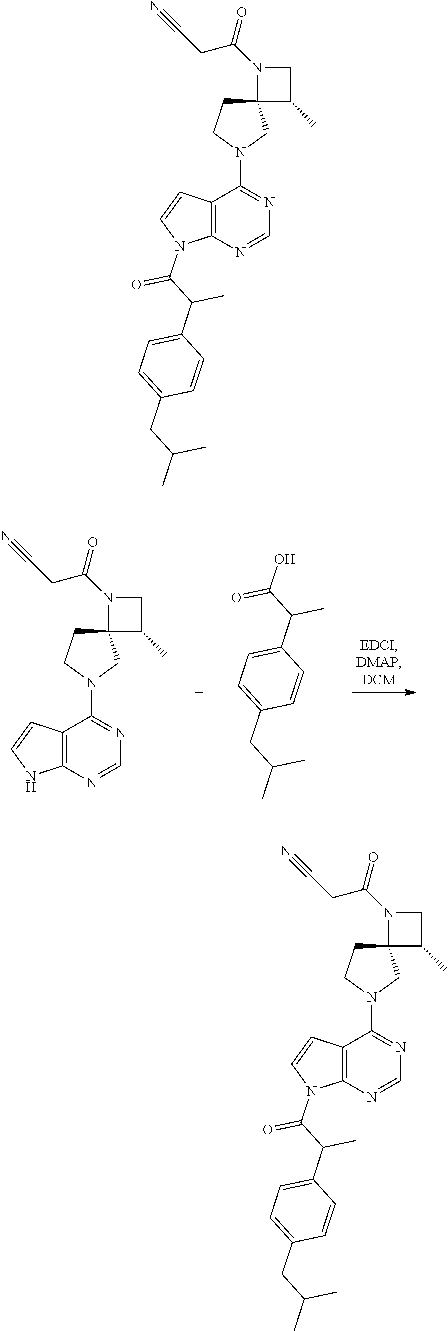 [SMILES: CC(C)Cc1ccc(C(C)C(=O)n2ccc3c(N4CC[C@]5(C4)[C@@H](C)CN5C(=O)CC#N)ncnc32)cc1 ; C[C@H]1CN(C(=O)CC#N)[C@]12CCN(c1ncnc3[nH]ccc13)C2 ; CC(C)Cc1ccc(C(C)C(=O)O)cc1 ; CC(C)Cc1ccc(C(C)C(=O)n2ccc3c(N4CC[C@]5(C4)[C@@H](C)CN5C(=O)CC#N)ncnc32)cc1]

### Synthesis of 3-((3S, 4R)-6-(7-(2-(4-isobutylphenyl) propanoyl)-7H-pyrrolo [2, 3-d] pyrimidin-4-yl)-3-methyl-1, 6-diazaspiro [3.4] oct-1-yl)-3-oxopropanenitrile

3-((3S, 4R)-3-methyl-6-(7H-pyrrolo [2, 3-d] pyrimidin-4-yl)-1, 6-diazaspiro [3.4] oct-1-yl)-3-oxopropanenitrile (Delgocitinib, 75 mg, 0.24 mmol), 4-dimethylamino pyridine (DMAP, 30 mg, 0.24 mmol), 2-(4-isobutylphenyl) propanoic acid (ibuprofen, 65 mg, 0.31 mmol) and 1-(3-dimethylaminopropyl)-3-ethylcarbodiimide hydrochloride (EDCI, 70 mg, 0.36 mmol) were dissolved in dichloromethane (1 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=30:1 to 10:1) to give the title compound as a white solid, 0.081 g, 67.7% yield. MS (m/z): [M+H]⁺ calcd for C₂₉H₃₄N₆O₂, 499.27, found 499.2.1H NMR (400 MHz, DMSO-d₆) δ 8.37 (s, 1H), 7.67 (d, J=4.1 Hz, 1H), 7.31 (d, J=8.1 Hz, 2H), 7.07 (d, J=8.0 Hz, 2H), 6.90 (d, J=4.1 Hz, 1H), 6.10 (q, J=6.8 Hz, 1H), 4.20-4.06 (m, 2H), 4.04-3.24 (m, 3H), 3.68 (d, J=3.3 Hz, 2H), 3.65-3.53 (m, 1H), 2.72-2.55 (m, 2H), 2.36 (d, J=7.1 Hz, 2H), 2.20 (s, 1H), 1.82-1.75 (m, 1H), 1.54 (d, J=6.9 Hz, 3H), 1.13 (d, J=7.0 Hz, 3H), 0.82 (d, J=6.6 Hz, 6H).

### Example 140

### 3-((3S, 4R)-6-(7-((S)-2-(4-isobutylphenyl) propanoyl)-7H-pyrrolo [2, 3-d] pyrimidin-4-yl)-3-methyl-1, 6-diazaspiro [3.4] oct-1-yl)-3-oxopropanenitrile

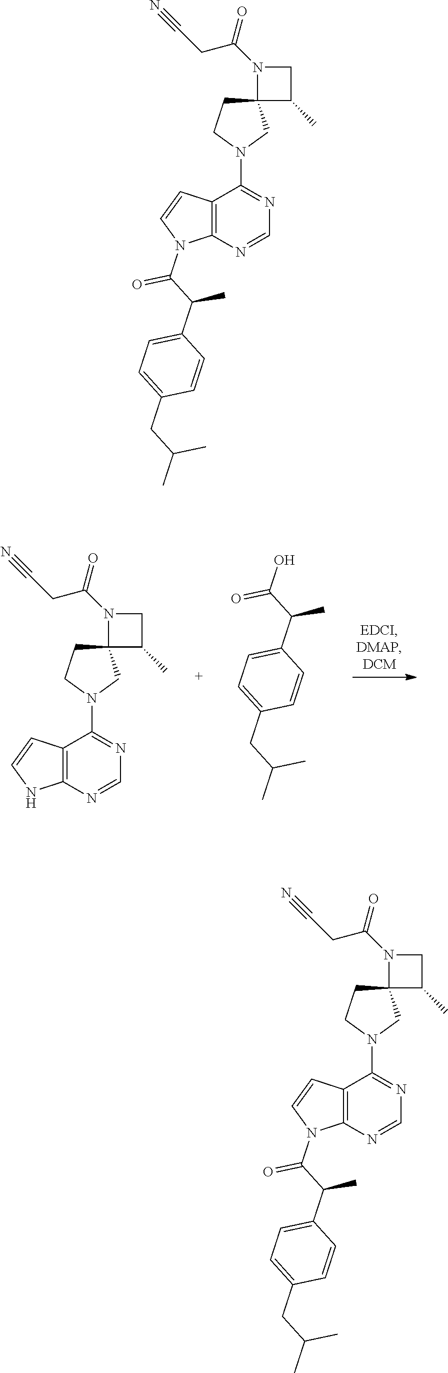 [SMILES: CC(C)Cc1ccc([C@H](C)C(=O)n2ccc3c(N4CC[C@]5(C4)[C@@H](C)CN5C(=O)CC#N)ncnc32)cc1 ; C[C@H]1CN(C(=O)CC#N)[C@]12CCN(c1ncnc3[nH]ccc13)C2 ; CC(C)Cc1ccc([C@H](C)C(=O)O)cc1 ; CC(C)Cc1ccc([C@H](C)C(=O)n2ccc3c(N4CC[C@]5(C4)[C@@H](C)CN5C(=O)CC#N)ncnc32)cc1]

### Synthesis of 3-((3S, 4R)-6-(7-((S)-2-(4-isobutylphenyl) propanoyl)-7H-pyrrolo [2, 3-d] pyrimidin-4-yl)-3-methyl-1, 6-diazaspiro [3.4] oct-1-yl)-3-oxopropanenitrile

3-((3S, 4R)-3-methyl-6-(7H-pyrrolo [2, 3-d] pyrimidin-4-yl)-1, 6-diazaspiro [3.4] oct-1-yl)-3-oxopropanenitrile (Delgocitinib, 75 mg, 0.24 mmol), 4-dimethylamino pyridine (DMAP, 30 mg, 0.24 mmol), (S)-(+)-2-(4-isobutylphenyl) propanoic acid((S)-(+)-ibuprofen, 65 mg, 0.31 mmol) and 1-(3-dimethylaminopropyl)-3-ethylcarbodiimide hydrochloride (EDCI, 70 mg, 0.36 mmol) were dissolved in dichloromethane (1 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=30:1 to 10:1) to give the title compound as a white solid, 0.071 g, 59.4% yield. MS (m/z): [M+H]⁺ calcd for C₂₉H₃₄N₆O₂, 499.27, found 499.3. ¹H NMR (400 MHz, DMSO-d₆) δ 8.37 (s, 1H), 7.67 (d, J=4.1 Hz, 1H), 7.31 (d, J=8.1 Hz, 2H), 7.07 (d, J=8.0 Hz, 2H), 6.90 (d, J=4.1 Hz, 1H), 6.10 (q, J=6.8 Hz, 1H), 4.20-4.05 (m, 2H), 4.04-3.24 (m, 3H), 3.68 (d, J=3.3 Hz, 2H), 3.65-3.53 (m, 1H), 2.73-2.55 (m, 2H), 2.36 (d, J=7.1 Hz, 2H), 2.20 (s, 1H), 1.77 (hept, J=6.8 Hz, 1H), 1.54 (d, J=6.9 Hz, 3H), 1.13 (d, J=7.0 Hz, 3H), 0.82 (d, J=6.6 Hz, 6H).

### Example 141

### 3-((3S, 4R)-6-(7-((S)-2-(6-methoxynaphthalen-2-yl) propanoyl)-7H-pyrrolo [2, 3-d] pyrimidin-4-yl)-3-methyl-1, 6-diazaspiro [3.4] octan-1-yl)-3-oxopropanenitrile

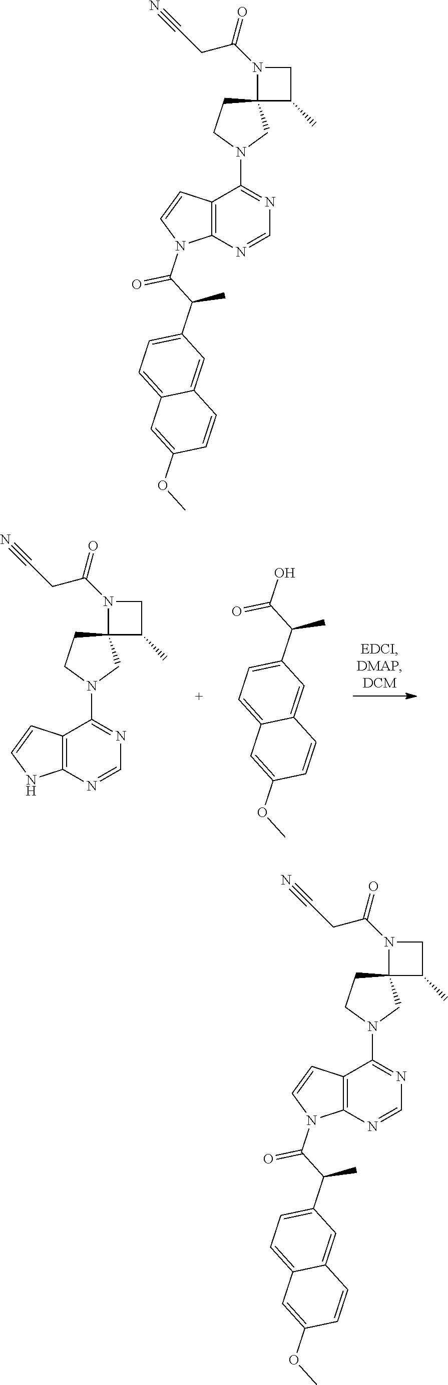

### Synthesis of 3-((3S, 4R)-6-(7-((S)-2-(6-methoxynaphthalen-2-yl) propanoyl)-7H-pyrrolo [2, 3-d] pyrimidin-4-yl)-3-methyl-1, 6-diazaspiro [3.4] octan-1-yl)-3-oxopropanenitrile

3-((3S, 4R)-3-methyl-6-(7H-pyrrolo [2, 3-d] pyrimidin-4-yl)-1, 6-diazaspiro [3.4] oct-1-yl)-3-oxopropanenitrile (Delgocitinib, 75 mg, 0.24 mmol), 4-dimethylamino pyridine (DMAP, 30 mg, 0.24 mmol), (S)-(+)-2-(6-methoxy-2-naphthyl) propanoic acid (Naproxen, 70 mg, 0.31 mmol) and 1-(3-dimethylaminopropyl)-3-ethylcarbodiimide hydrochloride (EDCI, 70 mg, 0.36 mmol) were dissolved in dichloromethane (1 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=30:1 to 10:1) to give the title compound as a white solid, 0.013 g, 10.3% yield. MS (m/z): [M+H]⁺ calcd for C₃₀H₃₀N₆O₃, 523.24, found 523.2. ¹H NMR (400 MHz, Chloroform-d) δ 8.38 (s, 1H), 7.76 (d, J=1.7 Hz, 1H), 7.67-7.56 (m, 2H), 7.51 (dd, J=8.6, 1.8 Hz, 1H), 7.40-7.35 (m, 1H), 7.08-6.98 (m, 2H), 6.53 (d, J=4.2 Hz, 1H), 6.19 (q, J=6.9 Hz, 1H), 4.30-3.91 (m, 2H), 3.82 (d, J=13.5 Hz, 5H), 3.69 (s, 1H), 3.63-3.57 (m, 2H), 3.20-3.05 (m, 1H), 2.84-2.74 (m, 1H), 2.63 (p, J=7.6, 7.2 Hz, 1H), 2.10-1.99 (m, 1H), 1.21-1.08 (m, 3H), 0.85-0.75 (m, 3H).

### Example 142

### 4-((4-(((3R, 4R)-1-(2-cyanoacetyl)-4-methylpiperidin-3-yl) (methyl) amino)-7H-pyrrolo [2, 3-d] pyrimidin-7-yl) methoxy)-2-methyl-N-(5-methylthiazol-2-yl)-2H-benzo [e] [1, 2] thiazine-3-carboxamide 1, 1-dioxide

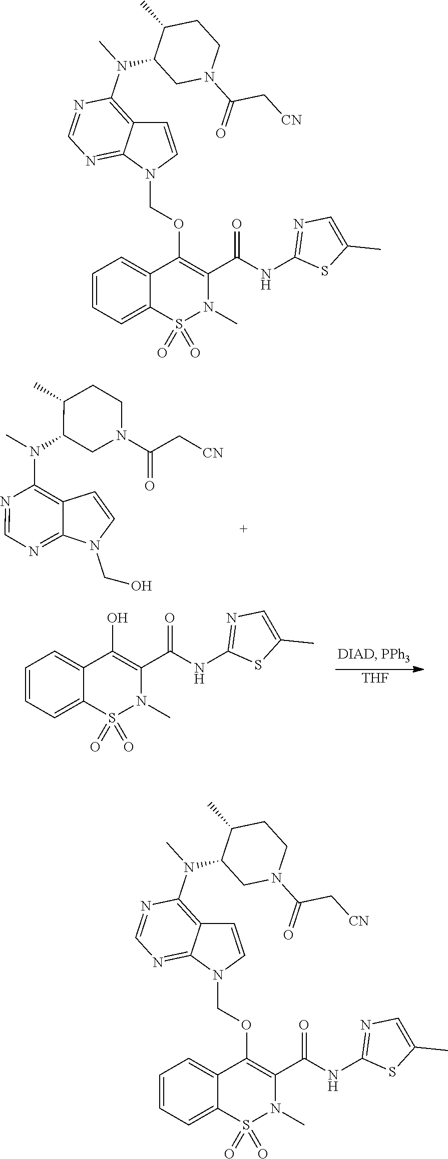

### Synthesis of 4-((4-(((3R, 4R)-1-(2-cyanoacetyl)-4-methylpiperidin-3-yl) (methyl) amino)-7H-pyrrolo [2, 3-d] pyrimidin-7-yl) methoxy)-2-methyl-N-(5-methylthiazol-2-yl)-2H-benzo [e] [1, 2] thiazine-3-carboxamide 1, 1-dioxide

4-Hydroxy-2-methyl-N-(5-methylthiazol-2-yl)-2H-benzo [e] [1, 2] thiazine-3-carboxamide 1, 1-dioxide (meloxicam, 246 mg, 0.7 mmol) and triphenylphosphine (PPh₃, 190 mg, 0.73 mmol) were added to tetrahydrofuran (3.4 mL) under nitrogen. After cooling to −10° C., diisopropyl azodicarboxylate (DIAD, 148 mg, 0.73 mmol) was added dropwise to the mixture with stirring. After stirring at −10° C. for 20 min, the mixture was allowed to warm to room temperature. 3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile (170 mg, 0.5 mmol) was added and the stirring continued. The reaction was monitored by TLC. After complete exhaust of the starting material (1 h), the solvent was evaporated under reduced pressure to give the crude product. The crude product was further separated by silica gel column chromatography (dichloromethane/acetonitrile=20:1 to 9:1) to give the title compound as a yellow solid, 0.009 g, 2.6% yield. MS (m/z): [M+H]⁺ calcd for C₃₁H₃₃N₉O₅S₂, 676.20, found 676.2. ¹H NMR (400 MHz, Chloroform-d) δ 14.01 (s, 1H), 8.28 (s, 1H), 8.03 (dd, J=7.6, 1.4 Hz, 1H), 7.84 (dd, J=7.4, 1.5 Hz, 1H), 7.73-7.52 (m, 3H), 7.30 (d, J=4.5 Hz, 1H), 6.51 (dd, J=11.0, 3.8 Hz, 1H), 6.42 (s, 2H), 5.01 (d, J=26.1 Hz, 1H), 4.04 (dd, J=13.2, 4.2 Hz, 1H), 3.86-3.68 (m, 1H), 3.64 (dd, J=13.2, 8.7 Hz, 1H), 3.56-3.39 (m, 3H), 3.29 (d, J=18.7 Hz, 3H), 3.09 (s, 3H), 2.53-2.39 (m, 1H), 2.21 (d, J=1.4 Hz, 3H), 2.00-1.80 (m, 1H), 1.73-1.63 (m, 1H), 1.02 (dd, J=9.5, 7.1 Hz, 3H).

### Example 143

### 3-((3S, 4R)-3-methyl-6-(7-(2-(4-(1-oxoisoindolin-2-yl) phenyl) butanoyl)-7H-pyrrolo [2, 3-d] pyrimidin-4-yl)-1, 6-diazaspiro [3.4] octan-1-yl)-3-oxopropanenitrile

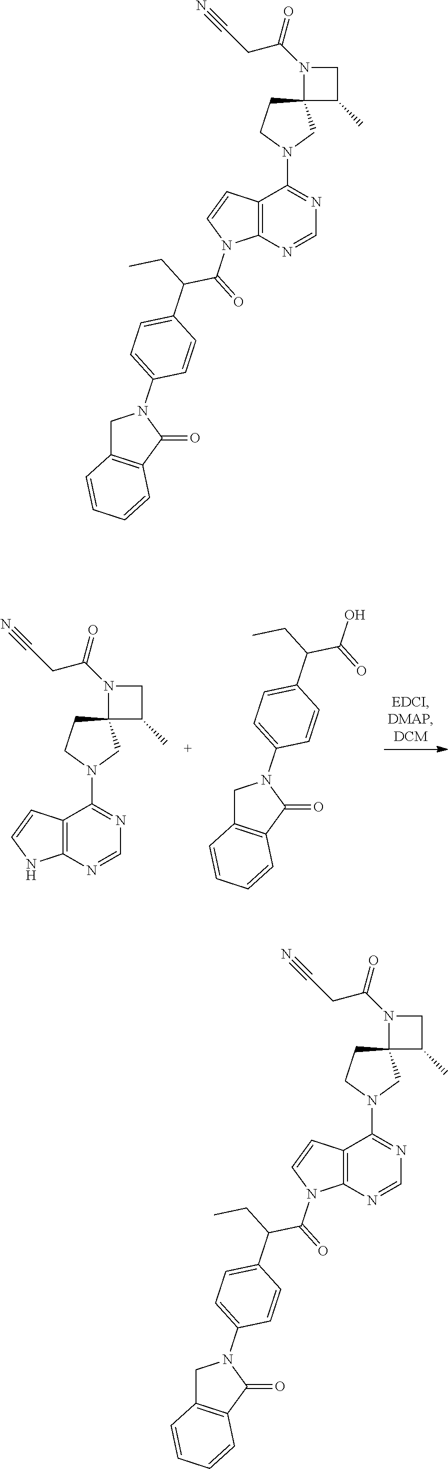 [SMILES: CCC(C(=O)n1ccc2c(N3CC[C@]4(C3)[C@@H](C)CN4C(=O)CC#N)ncnc21)c1ccc(N2Cc3ccccc3C2=O)cc1 ; C[C@H]1CN(C(=O)CC#N)[C@]12CCN(c1ncnc3[nH]ccc13)C2 ; CCC(C(=O)O)c1ccc(N2Cc3ccccc3C2=O)cc1 ; CCC(C(=O)n1ccc2c(N3CC[C@]4(C3)[C@@H](C)CN4C(=O)CC#N)ncnc21)c1ccc(N2Cc3ccccc3C2=O)cc1]

### Synthesis of 3-((3S, 4R)-3-methyl-6-(7-(2-(4-(1-oxoisoindolin-2-yl) phenyl) butanoyl)-7H-pyrrolo [2, 3-d] pyrimidin-4-yl)-1, 6-diazaspiro [3.4] octan-1-yl)-3-oxopropanenitrile

3-((3S, 4R)-3-methyl-6-(7H-pyrrolo [2, 3-d] pyrimidin-4-yl)-1, 6-diazaspiro [3.4] oct-1-yl)-3-oxopropanenitrile (delgocitinib, 75 mg, 0.24 mmol), 4-dimethylamino pyridine (DMAP, 30 mg, 0.24 mmol), 2-(4-(1-oxoisoindolin-2-yl) phenyl) butanoic acid (indobufen, 92 mg, 0.31 mmol) and 1-(3-dimethylaminopropyl)-3-ethylcarbodiimide hydrochloride (EDCI, 70 mg, 0.36 mmol) were dissolved in dichloromethane (1 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=30:1 to 10:1) to give the title compound as a white solid, 0.018 g, 12.7% yield. MS (m/z): [M+H]⁺ calcd for C₃₄H₃₃N₇O₃, 588.26, found 588.0. ¹H NMR (400 MHz, Chloroform-d) δ 8.37 (s, 1H), 7.86-7.79 (m, 1H), 7.70 (dd, J=8.7, 1.1 Hz, 2H), 7.60 (dd, J=4.2, 1.5 Hz, 1H), 7.56-7.46 (m, 3H), 7.46-7.39 (m, 2H), 6.55 (t, J=4.1 Hz, 1H), 5.93 (t, J=7.5 Hz, 1H), 4.74 (s, 2H), 4.26 (t, J=8.4 Hz, 1H), 4.16 (dd, J=12.0, 8.2 Hz, 1H), 4.12-3.94 (m, 2H), 3.73 (s, 1H), 3.62 (ddd, J=8.4, 6.0, 2.4 Hz, 1H), 3.14 (d, J=3.0 Hz, 2H), 2.81 (tt, J=13.0, 5.9 Hz, 1H), 2.67 (s, 1H), 2.22 (dt, J=14.2, 7.3 Hz, 1H), 2.09 (s, 1H), 1.96-1.86 (m, 1H), 1.16 (dd, J=7.1, 4.4 Hz, 3H), 0.92 (td, J=7.3, 1.2 Hz, 3H).

### Example 144

### (4-((3S, 4R)-1-(2-cyanoacetyl)-3-methyl-1, 6-diazaspiro [3.4] octan-6-yl)-7H-pyrrolo [2, 3-d] pyrimidin-7-yl) methyl (S)-2-(4-isobutylphenyl) propanoate

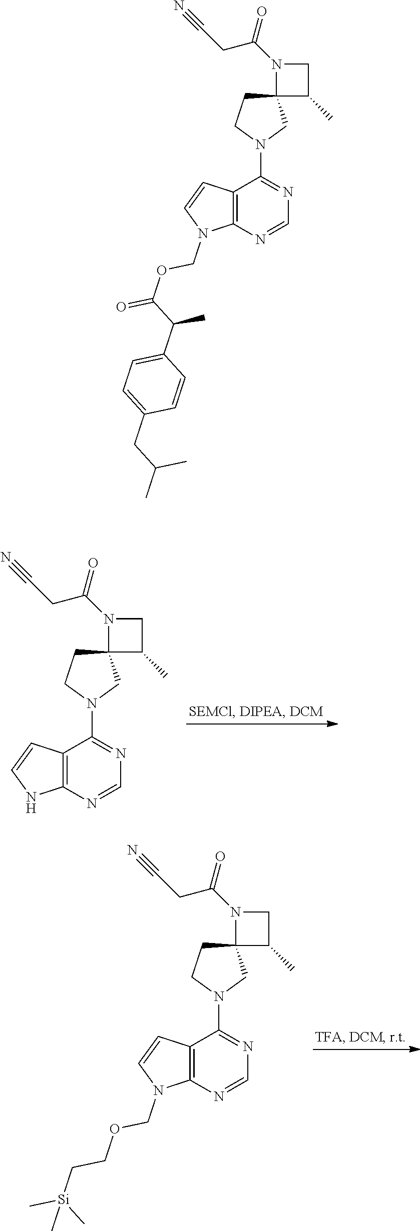

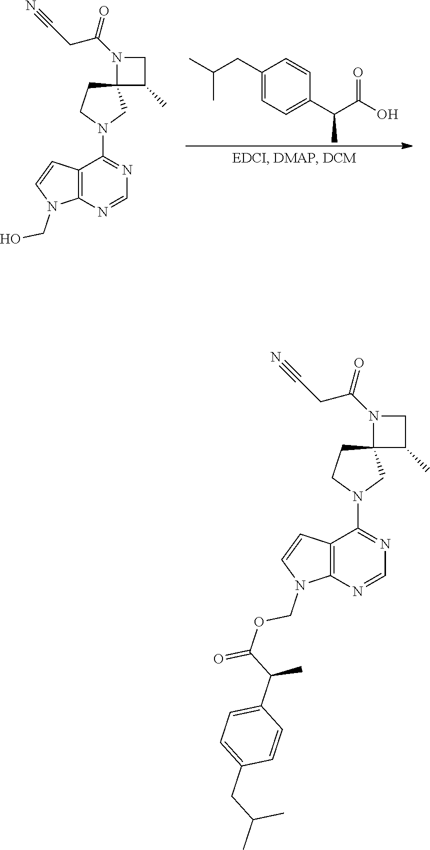 [SMILES: C[C@H]1CN(C(=O)CC#N)[C@]12CCN(c1ncnc3c1ccn3CO)C2 ; CC(C)Cc1ccc([C@H](C)C(=O)O)cc1 ; CC(C)Cc1ccc([C@H](C)C(=O)OCn2ccc3c(N4CC[C@]5(C4)[C@@H](C)CN5C(=O)CC#N)ncnc32)cc1]

### First Step: Synthesis of 3-((3S, 4R)-3-methyl-6-(7-((2-(trimethylsilyl) ethoxy) methyl)-7H-pyrrolo [2,3-d] pyrimidin-4-yl)-1, 6-diazaspiro [3.4] oct-1-yl)-3-oxopropanenitrile

Under nitrogen protection, 3-((3S, 4R)-3-methyl-6-(7H-pyrrolo [2, 3-d] pyrimidin-4-yl)-1, 6-diazaspiro [3.4] oct-1-yl)-3-oxopropanenitrile (Delgocitinib, 200 mg, 0.644 mmol) and N, N-diisopropylethyl amine (0.17 mL, 0.966 mmoL) were added in dichloromethane (2 mL). After stirring at room temperature for half an hour, (2-(chloromethoxy) ethyl) trimethylsilane (140 mg, 0.838 mmol) was added in an ice water, and stirring was continued at room temperature for 3 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=200:1 to 20:1) to give the title compound, 0.234 g, 82.5% yield. MS (m/z): [M+H]⁺ calcd for C₂₂H₃₂N₆O₂Si, 441.24; found, 441.2.

### Step 2: Synthesis of 3-((3S, 4R)-6-(7-(hydroxymethyl)-7H-pyrrolo [2, 3-d] pyrimidin-4-yl)-3-methyl-1, 6-diazaspiro [3.4] octan-1-yl)-3-oxopropanenitrile

Trifluoroacetic acid (1 mL) was slowly added dropwise to a solution of 3-((3S, 4R)-3-methyl-6-(7-((2-(trimethylsilyl) ethoxy) methyl)-7H-pyrrolo [2, 3-d] pyrimidin-4-yl)-1, 6-diazaspiro [3.4] oct-1-yl)-3-oxopropanenitrile (0.234 g, 0.53 mmol) in dichloromethane (15 mL) under nitrogen in an ice-water bath. After half an hour, the ice-water bath was removed and the temperature was raised to room temperature and stirring was continued for 2 hours. Saturated sodium bicarbonate solution was added to the above reaction solution at 0° C. to adjust the pH to 8. Then the mixture was poured into a separating funnel for separation. The organic layer was washed with a saturated salt water solution and dried over anhydrous sodium sulfate. After the filtration, the solvent was evaporated under reduced pressure to obtain the crude product. The crude product was further separated by silica gel column chromatography (dichloromethane/methanol=50:1 to 9:1) to give the title compound, 0.055 g, 30.4% yield. MS (m/z): [M+H]⁺ calcd for C₁₇H₂₀N₆O₂, 341.16; found, 341.1.

### Step 3: Synthesis of (4-(1-((R)-2-cyano-1-cyclopentylethyl)-1H-pyrazol-4-yl)-7H-pyrrolo [2, 3-d] pyrimidin-7-yl) methyl (S)-2-(4-isobutylphenyl) propanoate

3-((3S, 4R)-6-(7-(hydroxymethyl)-7H-pyrrolo [2, 3-d] pyrimidin-4-yl)-3-methyl-1, 6-diazaspiro [3.4] octan-1-yl)-3-oxopropanenitrile (55 mg, 0.16 mmol), 4-dimethylamino pyridine (DMAP, 19 mg, 0.16 mmol), (S)-(+)-2-(4-isobutylphenyl) propanoic acid((S)-(+)-ibuprofen, 32 mg, 0.17 mmol) and 1-(3-dimethylaminopropyl)-3-ethylcarbodiimide hydrochloride (EDCI, 50 mg, 0.26 mmol) were dissolved in a mixed solvent of dichloromethane (1 mL) and dimethylformamide (0.25 mL) and stirring was continued for 16 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=30:1 to 10:1) to give the title compound as a white solid, 0.02 g, 23.6% yield. MS (m/z): [M+H]⁺ calcd for C₃₀H₃₆N₆O₃, 529.28, found 529.2. ¹H NMR (400 MHz, Chloroform-d) δ 8.28 (s, 1H), 7.09-6.92 (m, 5H), 6.45 (d, J=3.7 Hz, 1H), 6.10 (d, J=10.6 Hz, 1H), 6.01 (d, J=10.6 Hz, 1H), 4.31-4.14 (m, 2H), 4.03 (d, J=11.9 Hz, 2H), 3.77 (s, 1H), 3.67-3.59 (m, 2H), 3.16 (s, 2H), 2.83 (dt, J=13.0, 7.5 Hz, 1H), 2.67 (h, J=7.0 Hz, 1H), 2.35 (d, J=7.2 Hz, 2H), 2.10 (s, 1H), 1.75 (dt, J=13.5, 6.7 Hz, 1H), 1.38 (d, J=7.2 Hz, 3H), 1.18 (d, J=2.8 Hz, 3H), 0.81 (d, J=6.7 Hz, 6H).

### Example 145

### 3-((3S, 4R)-6-(7-(2-(3-benzoylphenyl) propanoyl)-7H-pyrrolo [2, 3-d] pyrimidin-4-yl)-3-methyl-1, 6-diazaspiro [3.4] oct-1-yl)-3-oxopropanenitrile

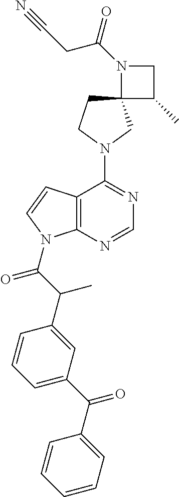 [SMILES: CC(C(=O)n1ccc2c(N3CC[C@]4(C3)[C@@H](C)CN4C(=O)CC#N)ncnc21)c1cccc(C(=O)c2ccccc2)c1]

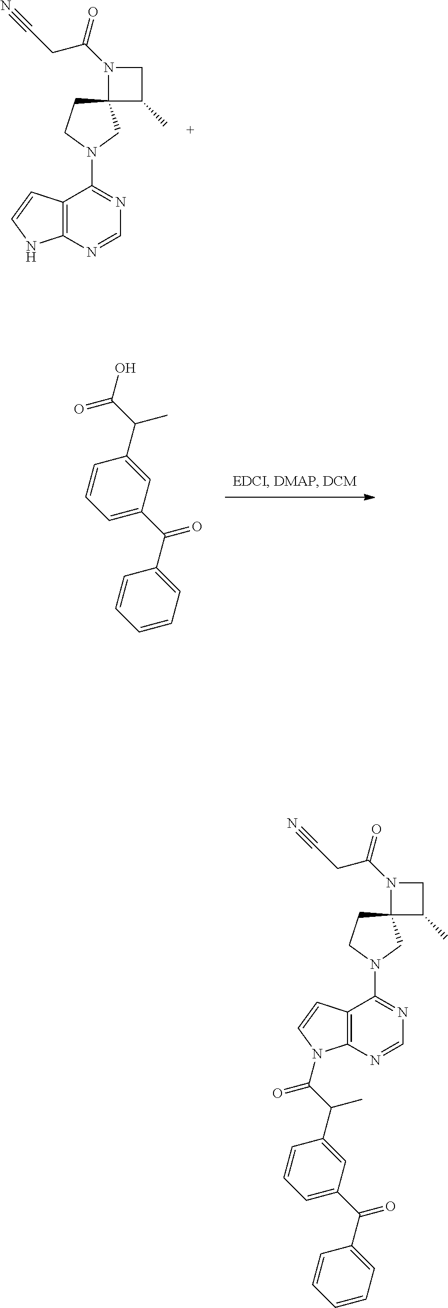 [SMILES: C[C@H]1CN(C(=O)CC#N)[C@]12CCN(c1ncnc3[nH]ccc13)C2 ; CC(C(=O)O)c1cccc(C(=O)c2ccccc2)c1 ; CC(C(=O)n1ccc2c(N3CC[C@]4(C3)[C@@H](C)CN4C(=O)CC#N)ncnc21)c1cccc(C(=O)c2ccccc2)c1]

### Synthesis of 3-((3S, 4R)-6-(7-(2-(3-benzoylphenyl) propanoyl)-7H-pyrrolo [2, 3-d] pyrimidin-4-yl)-3-methyl-1, 6-diazaspiro [3.4] oct-1-yl)-3-oxopropanenitrile

3-((3S, 4R)-3-methyl-6-(7H-pyrrolo [2, 3-d] pyrimidin-4-yl)-1, 6-diazaspiro [3.4] oct-1-yl)-3-oxopropanenitrile (Delgocitinib, 75 mg, 0.24 mmol), 4-dimethylamino pyridine (DMAP, 30 mg, 0.24 mmol), 2-(3-benzoylphenyl) propanoic acid (ketoprofen, 79 mg, 0.31 mmol) and 1-(3-dimethylaminopropyl)-3-ethylcarbodiimide hydrochloride (EDCI, 70 mg, 0.36 mmol) were dissolved in dichloromethane (1 mL) and stirred at room temperature for 18 hours. After completion of the reaction, the reaction solution was diluted with dichloromethane and washed with water and saturated salt water solution. The organic layer was dried over anhydrous sodium sulfate and filtered. The solvent was evaporated under reduced pressure to obtain the crude product. The crude product was further separated by silica gel column chromatography (dichloromethane/methanol=30:1 to 10:1) to give the title compound as a white solid, 0.038 g, 29% yield. MS (m/z): [M+H]⁺ calcd for C₃₂H₃₀N₆O₃, 547.24, found 547.1. ¹H NMR (400 MHz, Chloroform-d) δ 8.35 (s, 1H), 7.94 (s, 1H), 7.81-7.63 (m, 5H), 7.61-7.55 (m, 1H), 7.50-7.43 (m, 2H), 7.40 (t, J=7.7 Hz, 1H), 6.65 (dd, J=4.3, 1.5 Hz, 1H), 6.19 (q, J=7.0 Hz, 1H), 4.32 (t, J=8.4 Hz, 1H), 4.24 (dd, J=12.1, 7.2 Hz, 1H), 4.14 (s, 1H), 4.04 (dd, J=12.1, 8.4 Hz, 1H), 3.85-3.77 (m, 1H), 3.69 (dd, J=8.3, 5.9 Hz, 1H), 3.21 (d, J=2.7 Hz, 2H), 2.93-2.80 (m, 1H), 2.78-2.68 (m, 1H), 2.17 (dq, J=13.2, 7.3 Hz, 1H), 1.65 (d, J=7.0 Hz, 3H), 1.26-1.19 (m, 3H).

### Example 146

### 4-((4-(((3R, 4R)-1-(2-cyanoacetyl)-4-methylpiperidin-3-yl) (methyl) amino)-7H-pyrrolo [2, 3-d] pyrimidin-7-yl) methoxy)-2-methyl-N-(pyridin-2-yl)-2H-thieno [3, 2-e] [1, 2] thiazine-3-carboxamide 1, 1-dioxide

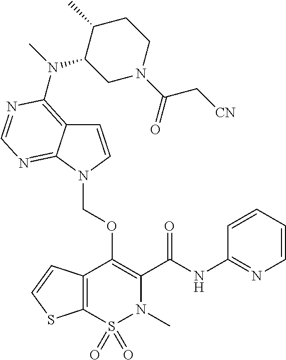 [SMILES: C[C@@H]1CCN(C(=O)CC#N)C[C@@H]1N(C)c1ncnc2c1ccn2COC1=C(C(=O)Nc2ccccn2)N(C)S(=O)(=O)c2sccc21]

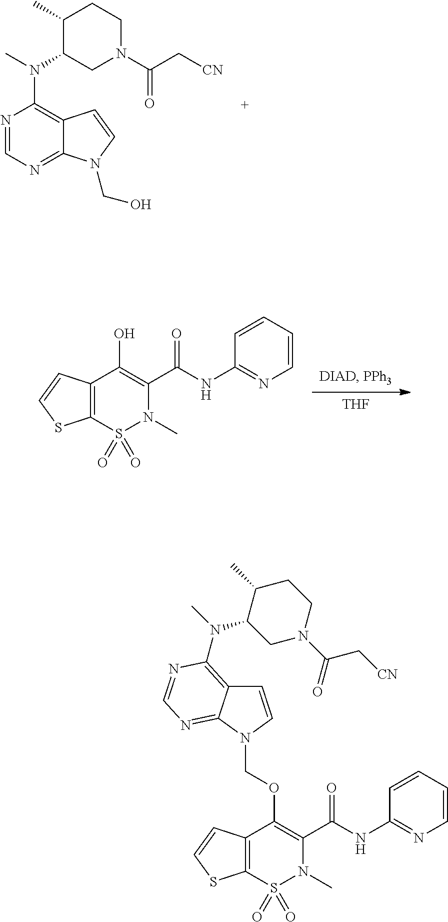 [SMILES: C[C@@H]1CCN(C(=O)CC#N)C[C@@H]1N(C)c1ncnc2c1ccn2CO ; CN1C(C(=O)Nc2ccccn2)=C(O)c2ccsc2S1(=O)=O ; C[C@@H]1CCN(C(=O)CC#N)C[C@@H]1N(C)c1ncnc2c1ccn2COC1=C(C(=O)Nc2ccccn2)N(C)S(=O)(=O)c2sccc21 ; "THF"]

### Synthesis of 4-((4-(((3R, 4R)-1-(2-cyanoacetyl)-4-methylpiperidin-3-yl) (methyl) amino)-7H-pyrrolo [2, 3-d] pyrimidin-7-yl) methoxy)-2-methyl-N-(pyridin-2-yl)-2H-thieno [3, 2-e] [1, 2] thiazine-3-carboxamide 1, 1-dioxide

4-Hydroxy-2-methyl-N-(pyridin-2-yl)-2H-thieno [3, 2-e] [1, 2] thiazine-3-carboxamide 1, 1-dioxide (tenoxicam, 98 mg, 0.292 mmol) and triphenylphosphine (PPh₃, 230 mg, 0.876 mmol) were added to tetrahydrofuran (2 mL) under nitrogen. Diisopropyl azodicarboxylate (DIAD, 88 mg, 0.438 mmol) was added dropwise to the mixture with stirring. After stirring at −10° C. for 20 minutes, the mixture was allowed to warm to room temperature. 3-((3R, 4R)-3-((7-(hydroxymethyl)-7H-pyrrolo [2, 3-d] pyrimidin-4-yl) (methyl) amino)-4-methylpiperidin-1-yl)-3-oxopropanenitrile (100 mg, 0.292 mmol) was added and stirring was continued. The reaction was monitored by TLC. After complete exhaust of the starting material (1 h), the solvent was evaporated under reduced pressure to give the crude product. The crude product was further separated by silica gel column chromatography (dichloromethane/acetonitrile=20:1 to 9:1) to give the title compound as a yellow solid, 0.011 g, 5.6% yield. MS (m/z): [M+H]⁺ calcd for C₃₀H₃₁N₉O₅S₂, 662.19, found 662.2.1H NMR (400 MHz, DMSO-d6) δ 16.02 (s, 1H), 8.63 (t, J=10.2 Hz, 1H), 8.38-8.23 (m, 2H), 8.19-8.10 (m, 1H), 8.06 (dd, J=5.2, 2.2 Hz, 1H), 7.75 (dd, J=6.7, 3.8 Hz, 1H), 7.49 (dd, J=5.2, 1.8 Hz, 1H), 7.25-7.17 (m, 1H), 6.81-6.63 (m, 3H), 4.85 (s, 1H), 4.19-3.88 (m, 3H), 3.81-3.59 (m, 3H), 3.27 (d, J=3.8 Hz, 3H), 2.99 (d, J=2.0 Hz, 3H), 2.41-2.33 (m, 1H), 1.90-1.69 (m, 1H), 1.63-1.56 (m, 1H), 1.00 (d, J=7.1 Hz, 3H).

### Example 147

### 2-Methyl-4-((4-(methyl ((trans)-4-((N-methylsulfamoyl) methyl) cyclohexyl) amino)-7H-pyrrolo [2, 3-d] pyrimidin-7-yl) methoxy)-N-(pyridin-2-yl)-2H-benzo [e] [1, 2] thiazine-3-carboxamide 1, 1-dioxide

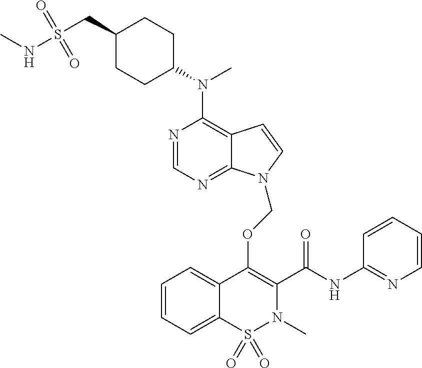

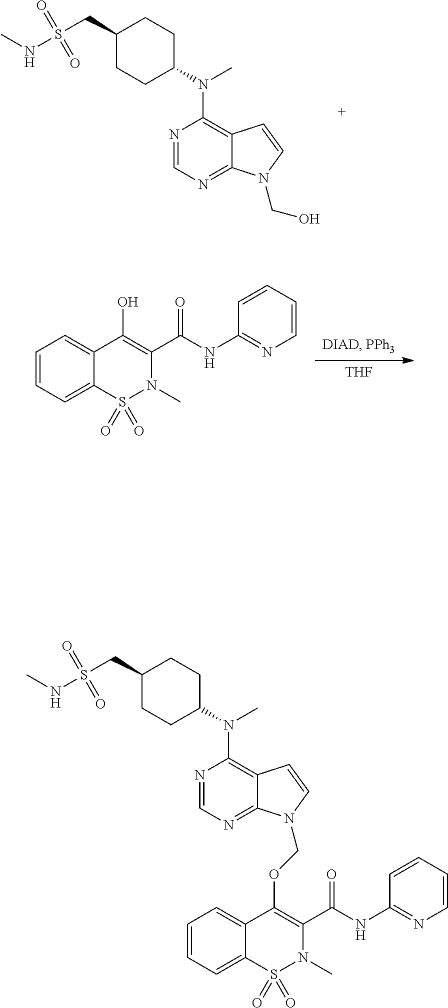

### Synthesis of 2-methyl-4-((4-(methyl ((trans)-4-((N-methylsulfamoyl) methyl) cyclohexyl) amino)-7H-pyrrolo [2, 3-d] pyrimidin-7-yl) methoxy)-N-(pyridin-2-yl)-2H-benzo [e] [1, 2] thiazine-3-carboxamide 1, 1-dioxide

4-Hydroxy-2-methyl-N-(pyridin-2-yl)-2H-benzo [e] [1, 2] thiazine-3-carboxamide 1, 1-dioxide (piroxicam, 122 mg, 0.368 mmol) and triphenylphosphine (PPh₃, 214 mg, 0.817 mmol) were added to tetrahydrofuran (3 mL) under nitrogen. After cooling to −10° C., diisopropyl azodicarboxylate (DIAD, 124 mg, 0.613 mmol) was added dropwise to the mixture with stirring. After stirring at −10° C. for 20 minutes, the mixture was allowed to warm to room temperature. 1-((Trans)-4-((7-(hydroxymethyl)-7H-pyrrolo [2, 3-d] pyrimidin-4-yl) (methyl) amino) cyclohexyl)-N-methylmethane sulfonamide (150 mg, 0.409 mmol) was added and the stirring was continued. The reaction was monitored by TLC. After complete exhaust of the starting material, the solvent was evaporated under reduced pressure to give the crude product. The crude product was further separated by silica gel column chromatography (dichloromethane/acetonitrile=20:1 to 9:1) to give the title compound as a yellow solid, 0.012 g, 4.8% yield. MS (m/z): [M+H]⁺ calcd for C₃₁H₃₆N₈O₆S₂, 681.22, found 681.2. ¹H NMR (400 MHz, Chloroform-d) δ 15.31 (s, 1H), 8.59 (d, J=6.6 Hz, 1H), 8.39 (d, J=9.1 Hz, 1H), 8.32 (s, 1H), 8.10 (dd, J=7.7, 1.4 Hz, 1H), 8.02 (s, 1H), 7.90 (dd, J=7.6, 1.4 Hz, 1H), 7.77-7.61 (m, 3H), 6.85-6.65 (m, 2H), 6.55 (d, J=3.8 Hz, 1H), 5.40-5.31 (m, 2H), 4.69 (s, 1H), 3.19 (s, 3H), 3.10 (s, 3H), 2.95 (d, J=6.2 Hz, 2H), 2.82 (d, J=5.3 Hz, 3H), 2.19-2.11 (m, 2H), 2.02-1.94 (m, 1H), 1.90-1.65 (m, 4H), 1.37-1.27 (m, 2H).

### Example 148

### 4-((4-(1-(3-(Cyanomethyl)-1-(ethylsulfonyl) azetidin-3-yl)-1H-pyrazol-4-yl)-7H-pyrrolo [2, 3-d] pyrimidin-7-yl) methoxy)-2-methyl-N-(pyridin-2-yl)-2H-thieno [3, 2-e] [1, 2] thiazine-3-carboxamide 1, 1-dioxide

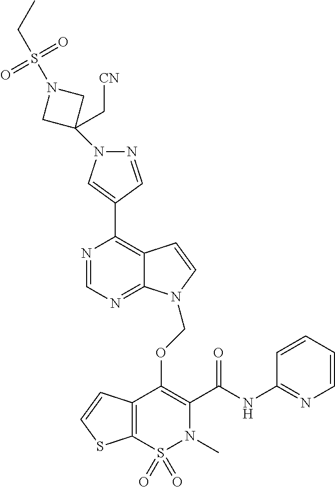 [SMILES: CCS(=O)(=O)N1CC(CC#N)(n2cc(-c3ncnc4c3ccn4COC3=C(C(=O)Nc4ccccn4)N(C)S(=O)(=O)c4sccc43)cn2)C1]

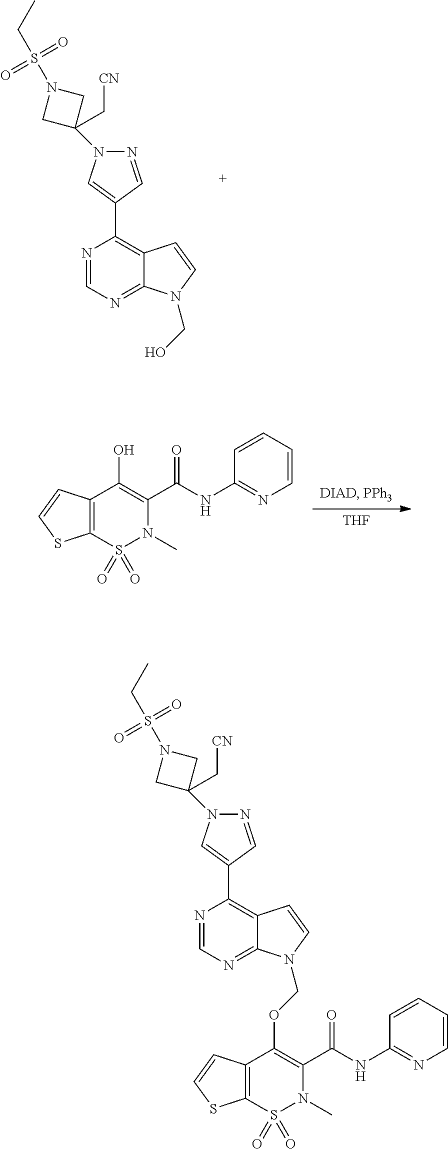 [SMILES: CCS(=O)(=O)N1CC(CC#N)(n2cc(-c3ncnc4c3ccn4CO)cn2)C1 ; CN1C(C(=O)Nc2ccccn2)=C(O)c2ccsc2S1(=O)=O ; CCS(=O)(=O)N1CC(CC#N)(n2cc(-c3ncnc4c3ccn4COC3=C(C(=O)Nc4ccccn4)N(C)S(=O)(=O)c4sccc43)cn2)C1 ; "THF"]

### Synthesis of 4-((4-(1-(3-(Cyanomethyl)-1-(ethylsulfonyl) azetidin-3-yl)-1H-pyrazol-4-yl)-7H-pyrrolo [2, 3-d] pyrimidin-7-yl) methoxy)-2-methyl-N-(pyridin-2-yl)-2H-thieno [3, 2-e] [1, 2] thiazine-3-carboxamide 1, 1-dioxide

4-Hydroxy-2-methyl-N-(pyridin-2-yl)-2H-thieno [3, 2-e] [1, 2] thiazine-3-carboxamide 1, 1-dioxide (tenoxicam, 75 mg, 0.224 mmol) and triphenylphosphine (PPh₃, 196 mg, 0.748 mmol) were added to tetrahydrofuran (2 mL) under nitrogen. After cooling to −10° C., diisopropyl azodicarboxylate (DIAD, 75 mg, 0.374 mmol) was added dropwise to the mixture with stirring. After stirring at −10° C. for 20 minutes, the mixture was allowed to warm to room temperature naturally. 2-(1-(Ethylsulfonyl)-3-(4-(7-(hydroxymethyl)-7H-)) pyrrolo [2, 3-d] pyrimidin-4-yl)-1H-pyrazol-1-yl) azetidin-3-yl) acetonitrile (100 mg, 0.249 mmol) was added and the stirring was continued. The reaction was monitored by TLC. After complete exhaust of the starting material, the solvent was evaporated under reduced pressure to give the crude product. The crude product was further separated by silica gel column chromatography (dichloromethane/acetonitrile=20:1 to 9:1) to give the title compound as a yellow solid, 0.008 g, 4.9% yield. MS (m/z): [M+H]⁺ calcd for C₃₀H₂₈N₁₀O₆S₃, 721.14, found 721.2. ¹H NMR (400 MHz, Chloroform-d) δ 13.45 (s, 1H), 8.65 (s, 1H), 8.46 (s, 1H), 8.25 (s, 1H), 8.18-8.08 (m, 2H), 7.65-7.56 (m, 2H), 7.44-7.31 (m, 2H), 6.90 (ddd, J=7.3, 4.9, 1.0 Hz, 1H), 6.60 (d, J=3.7 Hz, 1H), 6.17 (s, 2H), 4.56 (d, J=9.2 Hz, 2H), 4.18 (d, J=9.2 Hz, 2H), 3.35 (s, 2H), 3.12-2.99 (m, 5H), 1.35 (td, J=7.5, 3.8 Hz, 3H).

### Example 149

### 2-Methyl-4-((4-(methyl ((trans)-4-((N-methylsulfamoyl) methyl) cyclohexyl) amino)-7H-pyrrolo [2, 3-d] pyrimidin-7-yl) methoxy)-N-(pyridin-2-yl)-2H-thieno [3, 2-e] [1, 2] thiazine-3-carboxamide 1, 1-dioxide

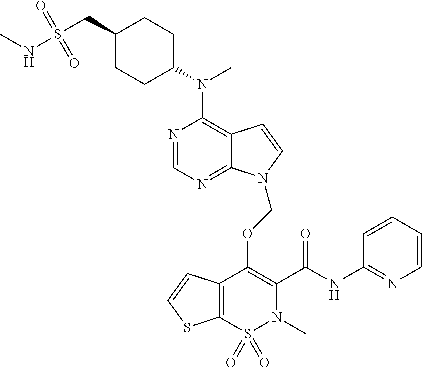

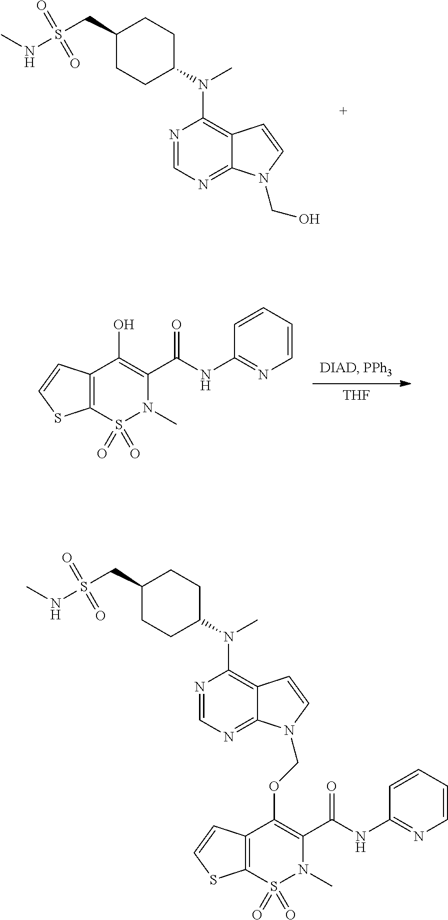

### Synthesis of 2-methyl-4-((4-(methyl ((trans)-4-((N-methylsulfamoyl) methyl) cyclohexyl) amino)-7H-pyrrolo [2, 3-d] pyrimidin-7-yl) methoxy)-N-(pyridin-2-yl)-2H-thieno [3, 2-e] [1, 2] thiazine-3-carboxamide 1, 1-dioxide

4-Hydroxy-2-methyl-N-(pyridin-2-yl)-2H-thieno [3, 2-e] [1, 2] thiazine-3-carboxamide 1, 1-dioxide (tenoxicam, 75 mg, 0.224 mmol) and triphenylphosphine (PPh₃, 196 mg, 0.748 mmol) were added to tetrahydrofuran (2 mL) under nitrogen. After cooling to −10° C., diisopropyl azodicarboxylate (DIAD, 75 mg, 0.374 mmol) was added dropwise to the mixture with stirring. After stirring at −10° C. for 20 minutes, the mixture was allowed to warm to room temperature naturally. 1-((Trans)-4-((7-(hydroxymethyl)-7H-pyrrolo [12, 3-d] pyrimidin-4-yl) (methyl) amino) cyclohexyl)-N-methylmethane sulfonamide (100 mg, 0.272 mmol) was added and the stirring was continued. The reaction was monitored by TLC. After complete exhaust of the starting material, the solvent was evaporated under reduced pressure to give the crude product. The crude product was further separated by silica gel column chromatography (dichloromethane/acetonitrile=20:1 to 9:1) to give the title compound as a yellow solid, 0.006 g, 3.9% yield. MS (m/z): [M+H]⁺ calcd for C₂₉H₃₄N₈O₆S₃, 687.18, found 687.2.1H NMR (400 MHz, Chloroform-d) δ 8.89 (s, 1H), 8.29-8.16 (m, 1H), 8.03 (s, 1H), 7.82 (dt, J=8.4, 1.0 Hz, 1H), 7.54 (ddd, J=8.5, 7.4, 1.9 Hz, 1H), 7.00 (ddd, J=7.3, 4.9, 1.0 Hz, 1H), 6.90 (d, J=3.8 Hz, 1H), 6.42 (d, J=3.8 Hz, 1H), 6.16 (s, 2H), 4.59-4.39 (m, 1H), 3.24 (d, J=5.0 Hz, 1H), 3.16 (s, 3H), 3.04 (d, J=14.0 Hz, 3H), 2.98 (d, J=6.3 Hz, 2H), 2.85 (dd, J=5.3, 3.6 Hz, 3H), 2.19 (d, J=12.8 Hz, 3H), 2.03 (q, J=6.2 Hz, 1H), 1.92-1.59 (m, 4H), 1.43-1.33 (m, 2H).

### Analysis Method for Hydrolysis Rate of Compounds of the Present Invention

### 1. Preparation of Standard Working Curve Solution

About 10 mg of JAK inhibitors (tofacitinib and baricitinib) reference substances were weighed into a 25 mL volumetric flask, acetonitrile/water=7/3 (v/v) was added to dissolve and dilute to the marked volume, serving as a stock solution. The stock solution was diluted with a suitable dilution factor to obtain linear solutions of each concentration: 0.0002 mg/mL, 0.0004 mg/mL, 0.004 mg/mL, 0.04 mg/mL, and 0.08 mg/mL.

About 10 mg of JAK inhibitors (Oclacitinib, Ruxolitinib, Delgocitinib, and Upadacitinib) reference substances were weighed into a 25 mL volumetric flask, acetonitrile/water=7/3 (v/v) was added to dissolve and dilute to the marked volume, serving as a stock solution. The stock solution was diluted with a suitable dilution factor to obtain linear solutions of each concentration: 0.0002 mg/mL, 0.0004 mg/mL, 0.004 mg/mL, 0.04 mg/mL, and 0.08 mg/mL.

### 2. Preparation of Sample Solution

About 5 mg of the sample (the compound prepared in the present invention) was weighed into a 20 mL volumetric flask, acetonitrile/water=7/3 (v/v) was added to dissolve and dilute to the marked volume, serving as a stock solution. An appropriate amount of HPLC was loaded as the 0-day sample. The test solution was sealed with a sealing film and placed in a 37° C. constant temperature shaker. The test samples were taken at the same time on days 1, 2, 3, and 4 respectively. the release amount of JAK inhibitor in the sample was calculated according to the sample weight and JAK inhibitor standard curve.

### 3. Calculation Formula

$$\% Release of JAK Inhibitor e = 100 \% \times ( ( {A}_{JAK Inhibitor} - b ) / a ) / ( ( W / 20 \times M 2 / M 1 )$$

wherein

| | |
|---|---|
| A_{JAK inhibitor} is the peak area of JAK | a is the slope of the standard |
| inhibitor in the sample chromatogram | curve |
| b is the intercept of the standard curve | W is the sample weight (mg) |
| M1 is the molecular weight of the | M2 is the molecular weight of |
| sample | the JAK inhibitor |

### 4. Chromatographic Conditions

The chromatographic conditions of the test are shown in Table 2.

TABLE 2
Chromatographic conditions

| Chromatographic | WatersXbridge Shield RP18, 150 × 4.6 mm, 3.5 μm | | | | |
|---|---|---|---|---|---|
| column | | | | | |
| Flow rate (LPM) | 0.8 | mL/min | Column temperature | 30° | C. |
| Detection | 220 | nm | Loading volume | 5 | μL |
| wavelength | | | | | |

| | |
|---|---|
| Mobile phase | 10 mM KH₂PO₄(Adjust pH6.5 with KOH)/ACN = 9/1(v/v); B: ACN |

| | Time | 0 | 10 | 16 | 17 | 22 |
|---|---|---|---|---|---|---|
| Elution gradient | A % | 85 | 20 | 20 | 85 | 85 |
| | B % | 15 | 80 | 80 | 15 | 15 |

Table 3 shows the drug release rate results of the compound drugs prepared in the above examples of the present disclosure.

TABLE 3
Drug Release Rate Table

| Ex. No. | Release Rate (%/day) |
|---|---|
| Ex. 1 | 8.26 |
| Ex. 2 | 8.5 |
| Ex. 3 | 0.05 |
| Ex. 4 | 3.32 |
| Ex. 5 | 4.7 |
| Ex. 6 | 9.82 |
| Ex. 7 | 15.65 |
| Ex. 8 | 14.77 |
| Ex. 9 | 13.49 |
| Ex. 10 | 11.25 |
| Ex. 11 | 9.17 |
| Ex. 12 | 18.94 |
| Ex. 13 | 12.18 |
| Ex. 14 | 0.98 |
| Ex. 15 | 1.86 |
| Ex. 16 | 39.31 |
| Ex. 17 | 2.44 |
| Ex. 18 | 2.97 |
| Ex. 19 | 6.25 |
| Ex. 20 | 5.06 |
| Ex. 21 | 4.6 |
| Ex. 22 | 2.75 |
| Ex. 23 | 7.63 |
| Ex. 24 | 5.96 |
| Ex. 25 | 0.68 |
| Ex. 26 | 36.19 |
| Ex. 27 | 7.48 |
| Ex. 28 | 2.98 |
| Ex. 29 | 7.08 |
| Ex. 30 | 1.63 |
| Ex. 31 | 3.08 |
| Ex. 32 | 7.12 |
| Ex. 33 | 5.01 |
| Ex. 34 | 1.57 |
| Ex. 35 | 4.57 |
| Ex. 36 | 14.98 |
| Ex. 37 | 1.05 |
| Ex. 38 | 18.42 |
| Ex. 39 | 10.11 |
| Ex. 40 | 0.62 |
| Ex. 41 | 4.27 |
| Ex. 42 | 22.18 |
| Ex. 43 | 2.99 |
| Ex. 44 | 10.18 |
| Ex. 45 | 1.93 |
| Ex. 46 | 25.52 |
| Ex. 47 | 0.36 |
| Ex. 48 | 0.03 |
| Ex. 49 | 0.11 |
| Ex. 50 | 0.05 |
| Ex. 51 | 0.35 |
| Ex. 52 | 0.1 |
| Ex. 53 | 0.06 |
| Ex. 54 | 4.93 |
| Ex. 55 | 0.16 |
| Ex. 56 | 0.07 |
| Ex. 57 | 0.57 |
| Ex. 58 | 12.46 |
| Ex. 59 | 0.15 |
| Ex. 60 | <0.01 |
| Ex. 61 | <0.01 |
| Ex. 62 | 0.01 |
| Ex. 63 | 0.02 |
| Ex. 64 | 0.88 |
| Ex. 65 | 0.01 |
| Ex. 66 | 0.06 |
| Ex. 67 | 0.02 |
| Ex. 68 | 10.6 |
| Ex. 69 | 0.03 |
| Ex. 70 | 0.42 |
| Ex. 71 | 0.01 |
| Ex. 72 | <0.01 |
| Ex. 73 | 0.2 |
| Ex. 74 | 0.44 |
| Ex. 75 | 10.38 |
| Ex. 76 | 0.05 |
| Ex. 77 | 3.69 |
| Ex. 78 | 6.27 |
| Ex. 79 | 7.38 |
| Ex. 80 | 27.29 |
| Ex. 81 | 0.013 |
| Ex. 82 | 0.43 |
| Ex. 83 | 0.003 |
| Ex. 84 | 0.06 |
| Ex. 85 | 0.61 |
| Ex. 86 | <0.01 |
| Ex. 87 | 0.01 |
| Ex. 88 | 0.17 |
| Ex. 89 | 0.16 |
| Ex. 90 | 0.35 |
| Ex. 91 | 1.65 |
| Ex. 92 | 0.82 |
| Ex. 93 | 0.83 |
| Ex. 94 | 0.72 |
| Ex. 99 | 0.21 |
| Ex. 100 | 0.24 |
| Ex. 104 | 0.45 |
| Ex. 105 | 0.17 |
| Ex. 107 | 0.22 |
| Ex. 110 | 0.08 |
| Ex. 113 | 0.42 |
| Ex. 116 | 0.42 |
| Ex. 117 | 0.08 |
| Ex. 120 | 0.42 |
| Ex. 122 | 15.32 |
| Ex. 123 | 3.02 |
| Ex. 124 | 2.75 |
| Ex. 125 | 6.17 |
| Ex. 126 | 7.80 |
| Ex. 130 | 11.67 |
| Ex. 131 | 3.56 |
| Ex. 133 | 10.13 |
| Ex. 138 | 2.15 |
| Ex. 140 | 8.10 |
| Ex. 142 | 0.05 |
| Ex. 143 | 4.29 |
| Ex. 144 | 0.05 |

As can be seen from the results in Table 3 above, the coupling of an anti-inflammatory drug compound containing a carboxylic acid or hydroxyl group with a JAK inhibitor compound to form a coupling compound having an acyloxy group and or methoxy group according to the present invention has a high therapeutic effect and a special effect of controlled-release drug activity.

### Pharmacodynamic Evaluation Studies of Compounds of the Invention

### Efficacy Evaluation Study of Ointment Transdermal Patch Formulated by Compounds Using IMQ-Induced Psoriasis Mouse Model

### Experimental Design

BALB/c mice (male) aged 6-8 weeks were selected to have depilated back skin with an area of 2×3 cm. 5% IMQ cream 62.5 mg was applied once a day for 9 consecutive days for model induction. Mice were housed in an SPF-grade animal room in IVC cages at a temperature of 20-26° C. and a humidity of 40-70% under a 12-hour light-dark cycle. Diet ad libitum, except where fasting is required.

Mice were randomly divided into groups, a number of 8 mice, administration concentration 20 mg/g, administration amount 50 mg, administration route, and frequency: skin smear, BID. The positive control was compound dexamethasone acetate (Dex) cream which is a commercially available product with a Dex content of 0.75 mg/g (999 Piyanping, Sanjiu Pharmaceutical). Dosing started from day 1 (the first day) (the day of model induction) to day 9.

### 2. Preparation of Test Ointment

Topical preparations are mostly semi-solid dosage forms. The base and solvent of a semi-solid dosage form are generally widely used in medicine and daily chemical products. The excipients which can be added are also the common excipients in the manual of excipients. Therefore, the patent compounds are prepared into corresponding test ointments. The ointment compound was dissolved with diisopropyl adipate to prepare a vaseline-based ointment. In order to improve the stability of the ointment, microcrystalline cellulose was added as an excipient in the formulation. The white vaseline has a melting point of 45-60° C. The solution, white vaseline, and microcrystalline cellulose are thoroughly mixed while the vaseline is in a molten state. After cooling to solidification, it was divided into an aluminum ointment, and the specific weight ratios of base, solvent, and excipients are shown in Table 4.

TABLE 4
Composition of ointment formulations for efficacy experiments

| Cmpd No. | CPD-002 | CPD-017 | CPD-027 | CPD-028 | CPD-029 |
|---|---|---|---|---|---|
| Compound | 2 | 2 | 2 | 2 | 2 |
| percentage | | | | | |
| Solvent | 15 | 15 | 15 | 15 | 15 |
| percentage | | | | | |
| Matrix | 80 | 80 | 80 | 80 | 80 |
| percentage | | | | | |
| Excipient | 3 | 3 | 3 | 3 | 3 |
| Percentage | | | | | |
| Total (w/w) | 100 | 100 | 100 | 100 | 100 |

### 3. Model Evaluation

Dorsal modeling site skin PASI was performed on the experimental animals. The scoring criteria are shown in Table 5 below:

TABLE 5
Scoring criteria

| Score | Erythema | Dandruff | Skin thickening |
|---|---|---|---|
| 0 | Normal | Normal | Normal |
| 1 | Mild | Mild: small amount of shavings | Mild |
| 2 | Moderate | Moderate: a large amount of | Moderate |
| | | scurf | |
| 3 | Severe | Severe: skin scab | Severe |
| 4 | Extremely | Extremely severe: heavy scab | Extremely severe |
| | severe | | |

Statistical analysis of PASI scores was performed using Two-way ANOVA. All data were analyzed using GraphPad Prism 8.0 software with a p <0.05 significant difference (*, p <0.05; **, p <0.01).

### 4. Efficacy

### PASI Score Curve

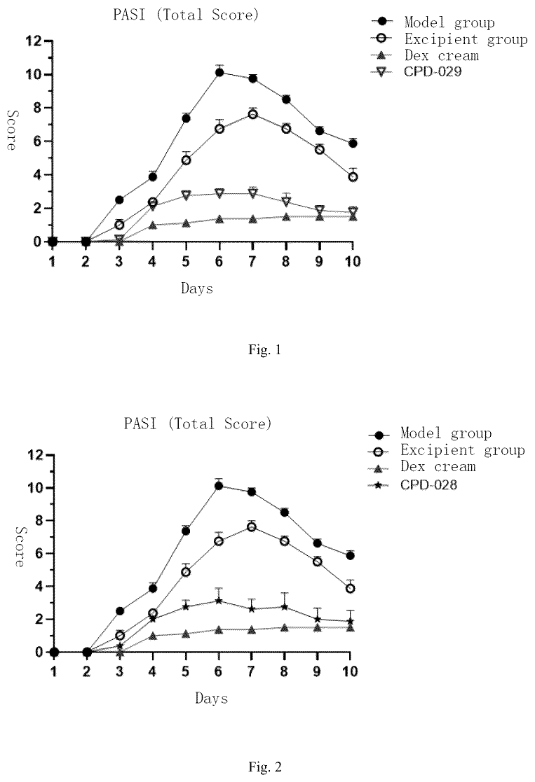

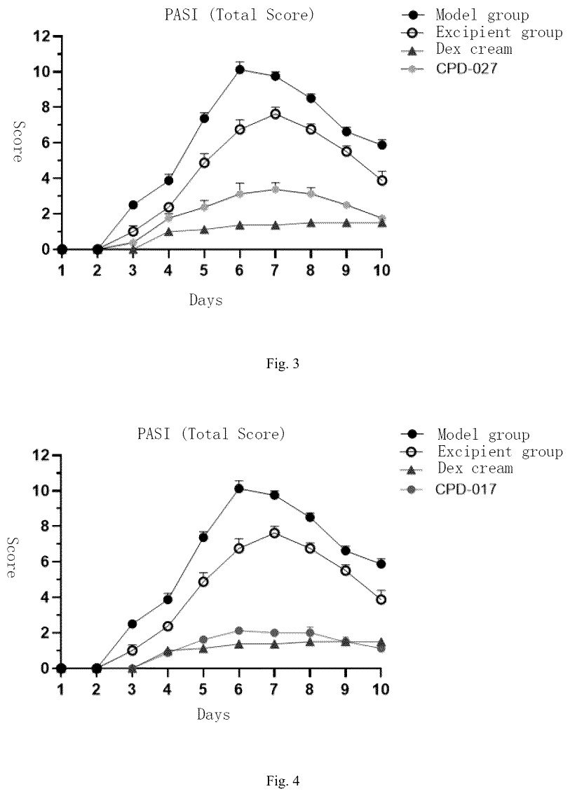

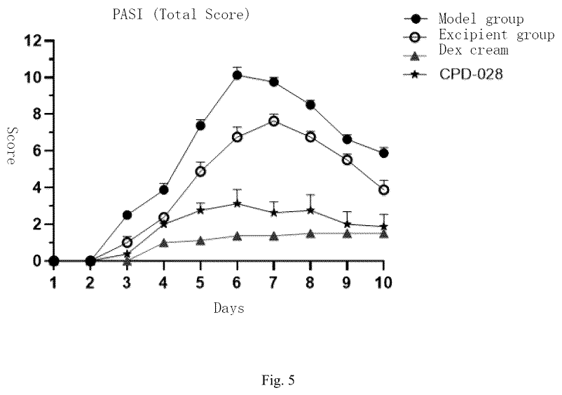

The PASI score curves of CPD-029, CPD-028, CPD-027, CPD-017, and CPD-002 compared with the model group, excipient group, and Dex cream group are shown in FIGS. 1, 2, 3, 4, and 5, respectively. Among them, the model group was treated with 62.5 mg of 5% IMQ cream applied to the back skin of mice once daily to induce psoriasis, and the excipient group was treated with 15% (w/w) diisopropyl adipate, 80% vaseline, and 3% microcrystalline cellulose.

As can be seen from FIGS. 1-5, the Dex cream group (0.75 mg/g) had a significant therapeutic effect on day 4-day 10 (days 4-10) compared with the excipient group; CPD-017, CPD-027, CPD-028, and CPD-029 had a significant therapeutic effect in day 5-day 10 (days 5-10); CPD-002 had a significant therapeutic effect in day 4 day 8 (days 4-8).

What is claimed is:

1. An anti-inflammatory compound, or a stereoisomer, tautomer, nitrogen oxide, prodrug, pharmaceutically acceptable salt, or solvate thereof, having a structure shown in general formula (I):
A-Y—B (I)
wherein, A is a group after dehydrogenation of an amine compound having JAK inhibitory activity;
Y is —(CH₂)—O— or —(CH₂)—;
B is a group formed by means of dehydroxylation of a carboxy-containing carboxylic acid compound B₁, or a group formed by means of dehydrogenation of a hydroxy-containing compound B₂; and wherein, in the case where the carboxylic acid B₁ is dehydroxylated to form a group, the Y group is —(CH₂)—O—; or in the case where the hydroxy-containing compound B₂ is dehydrogenated to form a group, the Y group is —(CH₂)—.

2. The compound, or the stereoisomer, tautomer, nitrogen oxide, prodrug, pharmaceutically acceptable salt, or solvate thereof of claim 1, wherein A- is a group after dehydrogenation of an amine compound selected from a group consisting of any one of the following groups: tofacitinib, baricitinib, oclacitinib, ruxolitinib, upadacitinib and delgocitinib:

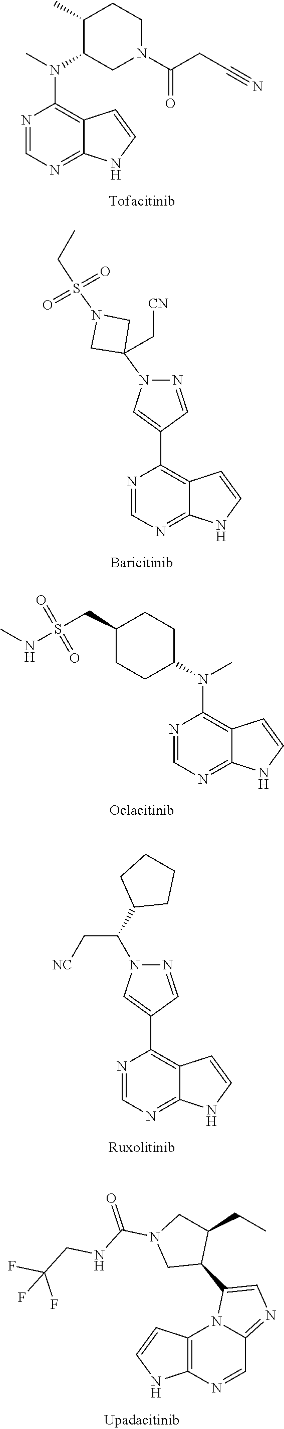

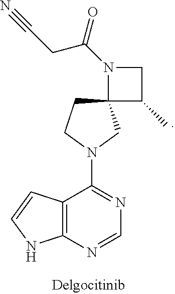 [SMILES: C[C@H]1CN(C(=O)CC#N)[C@]12CCN(c1ncnc3[nH]ccc13)C2]

3. The anti-inflammatory compound, or the stereoisomer, tautomer, nitrogen oxide, prodrug, pharmaceutically acceptable salt, or solvate thereof of claim 1, having a structure shown in general formulas (III) 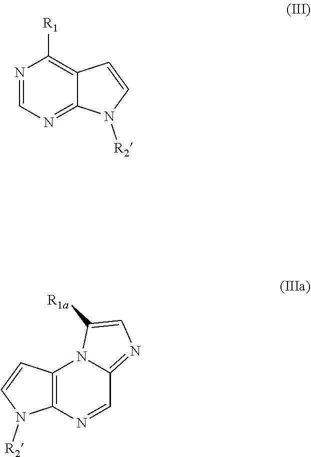
wherein R₁ is selected from pyrazolyl or pyrrolyl unsubstituted or substituted with Rₐ; or —N(CH₃)—Cy; R₁ₐ represents a pyrrolidine ring substituted by a halogen-substituted C₁-C₆ alkylaminoacyl group and/or by a C₁-C₆ alkyl group;
C_{y} is a five- or six-membered carbocyclic ring or a five- or six-membered nitrogen-containing heterocyclic ring unsubstituted or substituted by R_{b}; Rₐ and R_{b} are each independently groups containing at least one or two groups selected from a group consisting of an acyl group, a sulfonyl group, a cyano group, an amino group or a C₁-C₆ alkyl-substituted amino group, and a four-, five-, or six-membered nitrogen-containing heterocyclic group, or the nitrogen-containing heterocyclic group substituted with C₁-C₆ alkyl;
R₂′ in formula (III) and formula (IIIa) are both Y—B, B is B₁ or B is B₂; wherein the group —B₁ is a group formed by dehydroxylation of a carboxylic acid compound B₁ and Y— is (CH₂)—O—; the group —B₂ is a group formed by dehydrogenation of a hydroxyl-containing compound B₂ and Y— is —(CH₂)—; the group —B₁ is selected from R₄—Ar—R₃—CO—, wherein, R₃ is selected from C₁-C₆ alkylene; —NH—, R₅NH—, or C₁-C₆ alkylene substituted with a C₁-C₆ alkoxyamide group; or a direct connection wherein the Ar group is directly linked to —CO— and Ar is an aromatic ring group and R₄ is halogen, C₁-C₆ alkyl, C₁-C₆ alkoxy, C₁-C₆ alkyl containing C₁-C₆ cycloalkanoyl, C₁-C₆ alkylamido or aryl fused heterocyclic amido, C₁-C₆ carbonyloxy, halogen substituted benzoyl, C₁-C₆ alkyl or halogen substituted or unsubstituted phenoxy, C₁-C₆ alkyl or halogen substituted or unsubstituted phenyl or aryl fused heterocyclic ring, C₁-C₆ alkyl or halogen substituted or unsubstituted phenylamino, or R₄ can also be absent;
wherein the C₁-C₆ alkoxy can also form a bridged ring with Ar;
the group —B₂ is R_{c}—CO—NH—Rₐ, wherein R_{c} is a 4-hydroxy-benzothiazine dioxide-3-yl represented by the following structural formula (a), or a 4-hydroxy-Re substituted thienothiazine dioxide-3-yl represented by the following structural formula (b), wherein —CO—NH—R_{d} is bonded at the 3-position of the thiazine ring, 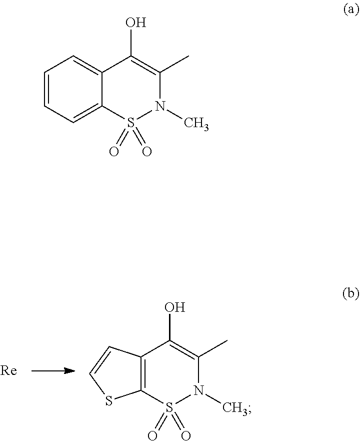
wherein R_{d} is thiazole, isothiazole, oxazole, isoxazole, or pyridine or the group thereof substituted with C₁-C₆ alkyl or halogen; Rₑ is C₁-C₆ alkyl or halogen; a arrow next to Rₑ in formula (b) indicates that its substitution position on the thiophene ring may be any carbon-linked hydrogen atom capable of undergoing substitution.

4. The anti-inflammatory compound, or the stereoisomer, tautomer, nitrogen oxide, prodrug, pharmaceutically acceptable salt, or solvate thereof of claim 3, wherein —B₁ is a group after dehydroxylation of any one of carboxylic acids selected from a group consisting of the following groups:
ibuprofen, (S)-(+)-ibuprofen, naproxen, fenoprofen, flurbiprofen, loxoprofen, ketoprofen, diclofenac, etodolac, actarit, indomethacin, N-Boc-L-phenylglycine, aspirin, indobufen, mefenamic acid and tolfenamic acid: 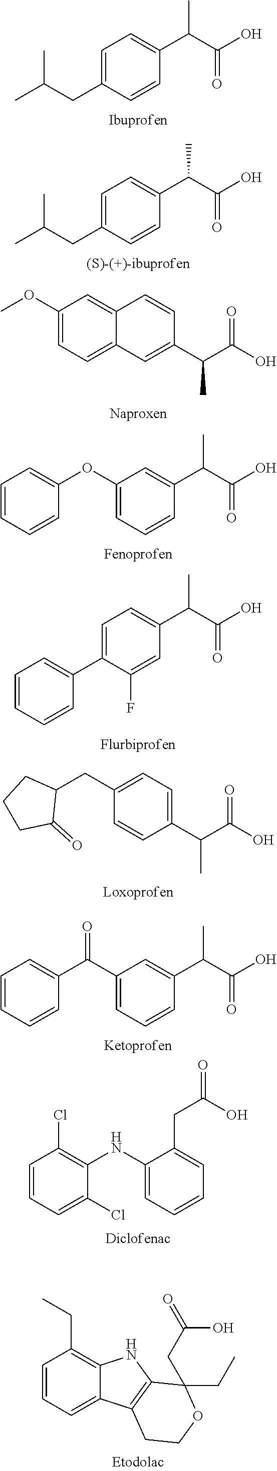 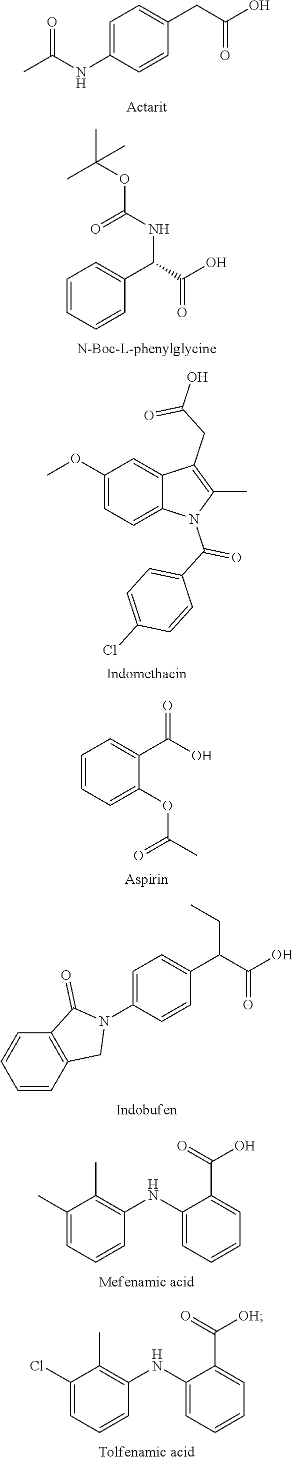
—B₂ is a group after dehydrogenation of a hydroxyl-containing compound of one of the following specific compounds:

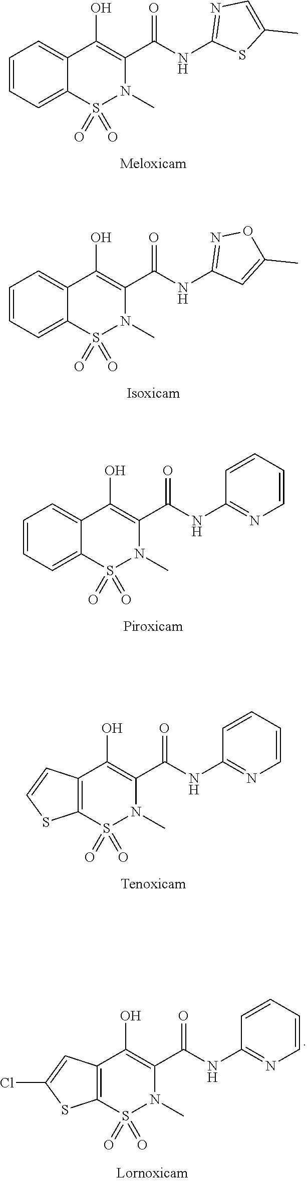 [SMILES: Cc1cnc(NC(=O)C2=C(O)c3ccccc3S(=O)(=O)N2C)s1 ; Cc1cc(NC(=O)C2=C(O)c3ccccc3S(=O)(=O)N2C)no1 ; CN1C(C(=O)Nc2ccccn2)=C(O)c2ccccc2S1(=O)=O ; CN1C(C(=O)Nc2ccccn2)=C(O)c2ccsc2S1(=O)=O ; CN1C(C(=O)Nc2ccccn2)=C(O)c2cc(Cl)sc2S1(=O)=O]

5. The anti-inflammatory compound, or the stereoisomer, tautomer, nitrogen oxide, prodrug, pharmaceutically acceptable salt, or solvate thereof of claim 3, obtained by a preparation method comprising the step of:
1) reacting A-CH₂—OH compound with an acyl chloride of B or directly with the B compound;
wherein the A-CH₂ OH compound is prepared by the following step 1): reacting the amine compound A to form the A-CH₂—OH compound, wherein A- is a group after dehydrogenation of an amine compound selected from a group consisting of any one of the following groups: tofacitinib, baricitinib, oclacitinib, ruxolitinib, upadacitinib and delgocitinib:

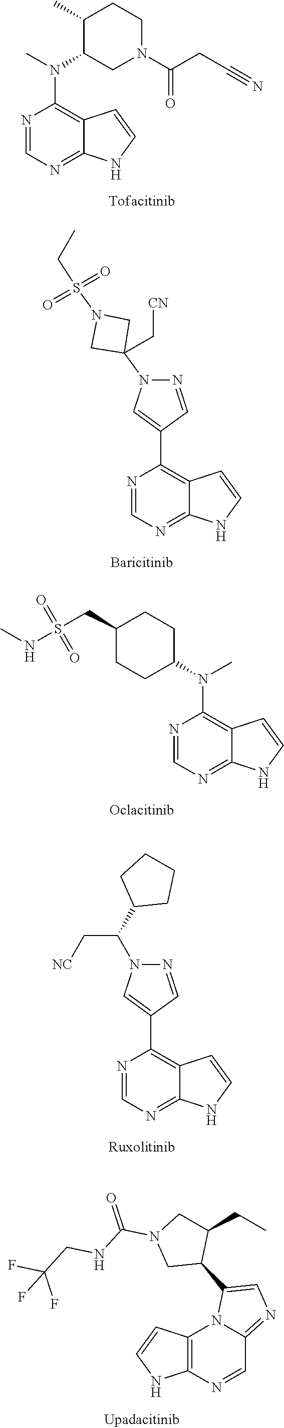

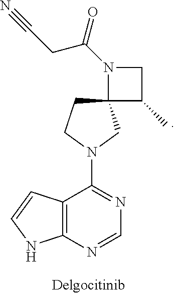 [SMILES: C[C@H]1CN(C(=O)CC#N)[C@]12CCN(c1ncnc3[nH]ccc13)C2]

6. The anti-inflammatory compound, or the stereoisomer, tautomer, nitrogen oxide, prodrug, pharmaceutically acceptable salt, or solvate thereof of claim 5, wherein the compound is any one of the following specific compounds:

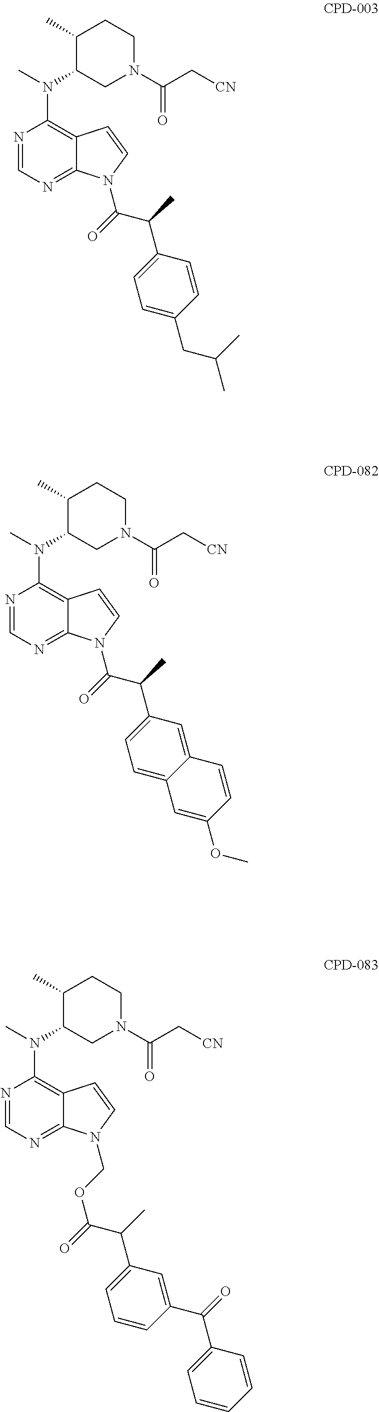 [SMILES: CC(C)Cc1ccc([C@H](C)C(=O)n2ccc3c(N(C)[C@H]4CN(C(=O)CC#N)CC[C@H]4C)ncnc32)cc1 ; COc1ccc2cc([C@H](C)C(=O)n3ccc4c(N(C)[C@H]5CN(C(=O)CC#N)CC[C@H]5C)ncnc43)ccc2c1 ; CC(C(=O)OCn1ccc2c(N(C)[C@H]3CN(C(=O)CC#N)CC[C@H]3C)ncnc21)c1cccc(C(=O)c2ccccc2)c1]

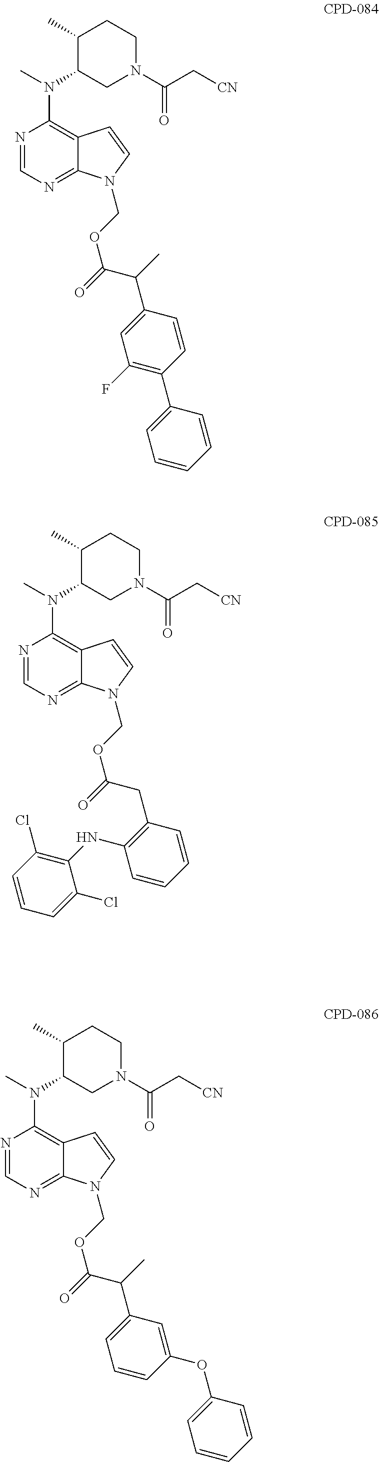 [SMILES: CC(C(=O)OCn1ccc2c(N(C)[C@H]3CN(C(=O)CC#N)CC[C@H]3C)ncnc21)c1ccc(-c2ccccc2)c(F)c1 ; C[C@@H]1CCN(C(=O)CC#N)C[C@@H]1N(C)c1ncnc2c1ccn2COC(=O)Cc1ccccc1Nc1c(Cl)cccc1Cl ; CC(C(=O)OCn1ccc2c(N(C)[C@H]3CN(C(=O)CC#N)CC[C@H]3C)ncnc21)c1cccc(Oc2ccccc2)c1]

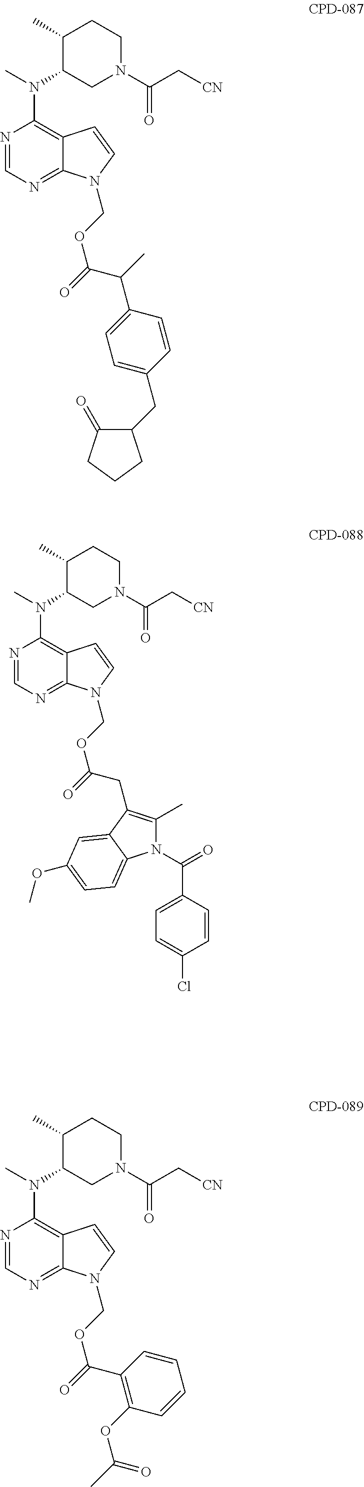 [SMILES: CC(C(=O)OCn1ccc2c(N(C)[C@H]3CN(C(=O)CC#N)CC[C@H]3C)ncnc21)c1ccc(CC2CCCC2=O)cc1 ; COc1ccc2c(c1)c(CC(=O)OCn1ccc3c(N(C)[C@H]4CN(C(=O)CC#N)CC[C@H]4C)ncnc31)c(C)n2C(=O)c1ccc(Cl)cc1 ; CC(=O)Oc1ccccc1C(=O)OCn1ccc2c(N(C)[C@H]3CN(C(=O)CC#N)CC[C@H]3C)ncnc21]

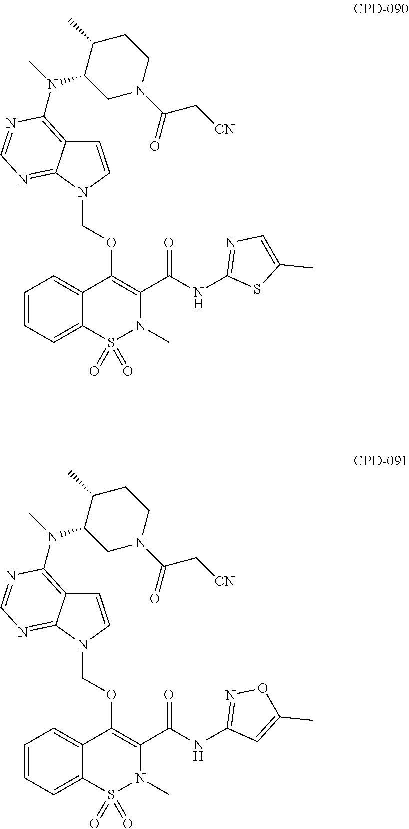 [SMILES: Cc1cnc(NC(=O)C2=C(OCn3ccc4c(N(C)[C@H]5CN(C(=O)CC#N)CC[C@H]5C)ncnc43)c3ccccc3S(=O)(=O)N2C)s1 ; Cc1cc(NC(=O)C2=C(OCn3ccc4c(N(C)[C@H]5CN(C(=O)CC#N)CC[C@H]5C)ncnc43)c3ccccc3S(=O)(=O)N2C)no1]

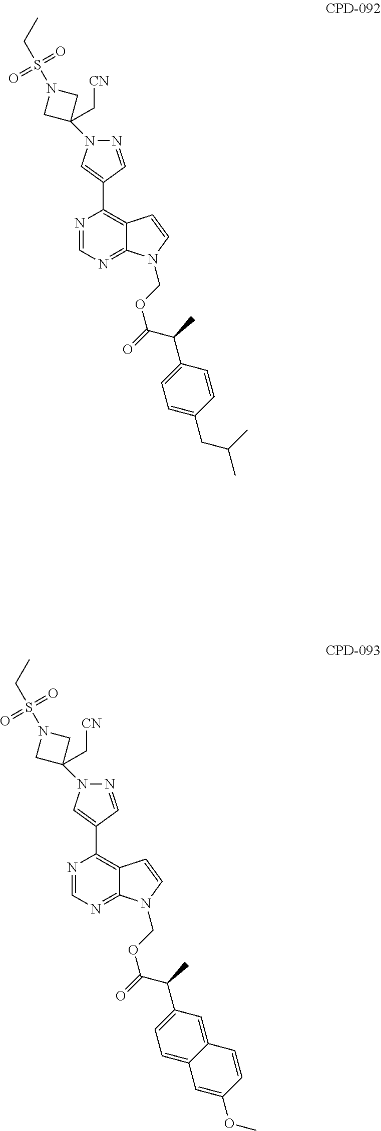 [SMILES: CCS(=O)(=O)N1CC(CC#N)(n2cc(-c3ncnc4c3ccn4COC(=O)[C@@H](C)c3ccc(CC(C)C)cc3)cn2)C1 ; CCS(=O)(=O)N1CC(CC#N)(n2cc(-c3ncnc4c3ccn4COC(=O)[C@@H](C)c3ccc4cc(OC)ccc4c3)cn2)C1]

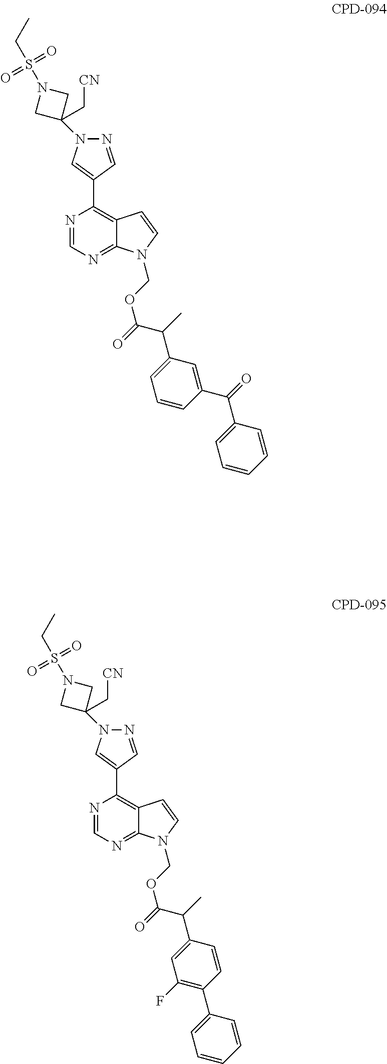 [SMILES: CCS(=O)(=O)N1CC(CC#N)(n2cc(-c3ncnc4c3ccn4COC(=O)C(C)c3cccc(C(=O)c4ccccc4)c3)cn2)C1 ; CCS(=O)(=O)N1CC(CC#N)(n2cc(-c3ncnc4c3ccn4COC(=O)C(C)c3ccc(-c4ccccc4)c(F)c3)cn2)C1]

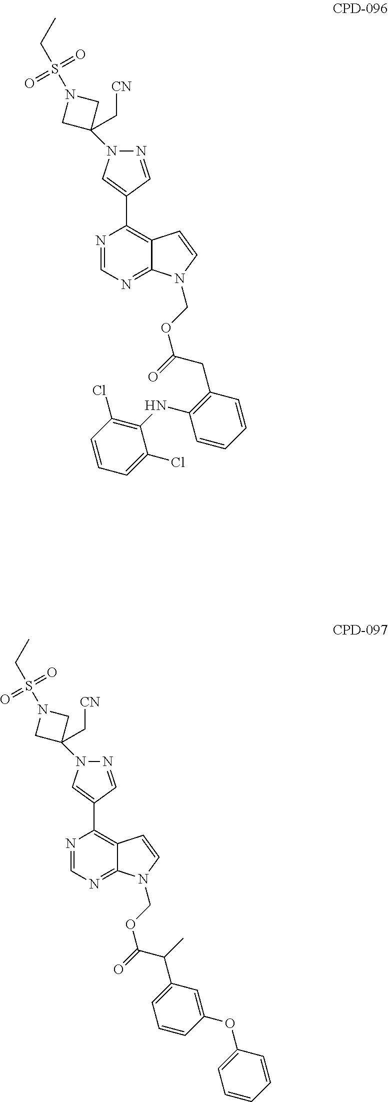 [SMILES: CCS(=O)(=O)N1CC(CC#N)(n2cc(-c3ncnc4c3ccn4COC(=O)Cc3ccccc3Nc3c(Cl)cccc3Cl)cn2)C1 ; CCS(=O)(=O)N1CC(CC#N)(n2cc(-c3ncnc4c3ccn4COC(=O)C(C)c3cccc(Oc4ccccc4)c3)cn2)C1]

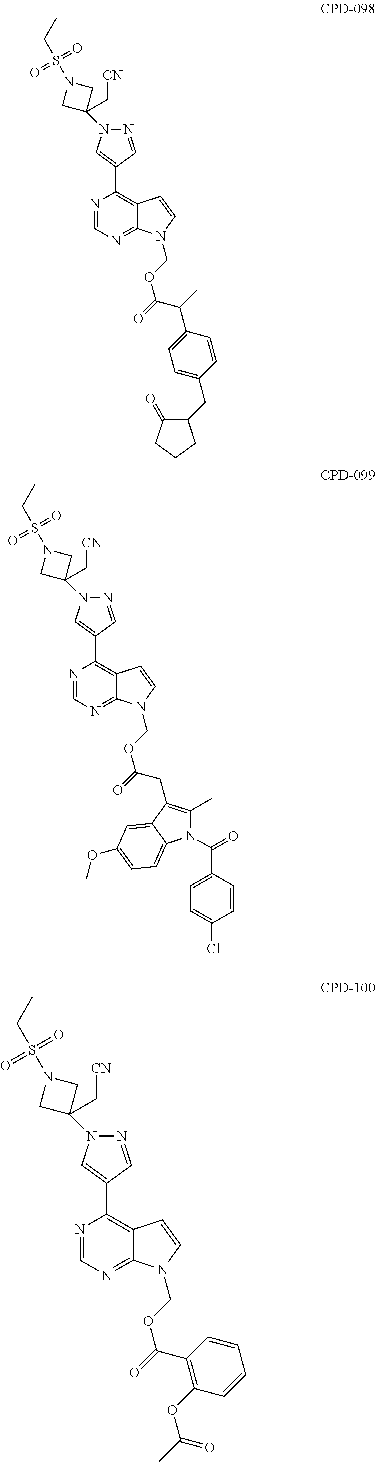 [SMILES: CCS(=O)(=O)N1CC(CC#N)(n2cc(-c3ncnc4c3ccn4COC(=O)C(C)c3ccc(CC4CCCC4=O)cc3)cn2)C1 ; CCS(=O)(=O)N1CC(CC#N)(n2cc(-c3ncnc4c3ccn4COC(=O)Cc3c(C)n(C(=O)c4ccc(Cl)cc4)c4ccc(OC)cc34)cn2)C1 ; CCS(=O)(=O)N1CC(CC#N)(n2cc(-c3ncnc4c3ccn4COC(=O)c3ccccc3OC(C)=O)cn2)C1]

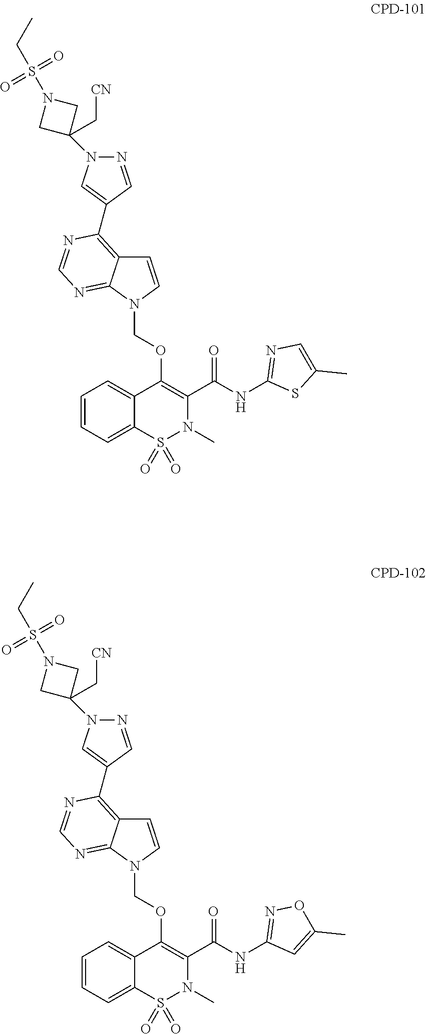 [SMILES: CCS(=O)(=O)N1CC(CC#N)(n2cc(-c3ncnc4c3ccn4COC3=C(C(=O)Nc4ncc(C)s4)N(C)S(=O)(=O)c4ccccc43)cn2)C1 ; CCS(=O)(=O)N1CC(CC#N)(n2cc(-c3ncnc4c3ccn4COC3=C(C(=O)Nc4cc(C)on4)N(C)S(=O)(=O)c4ccccc43)cn2)C1]

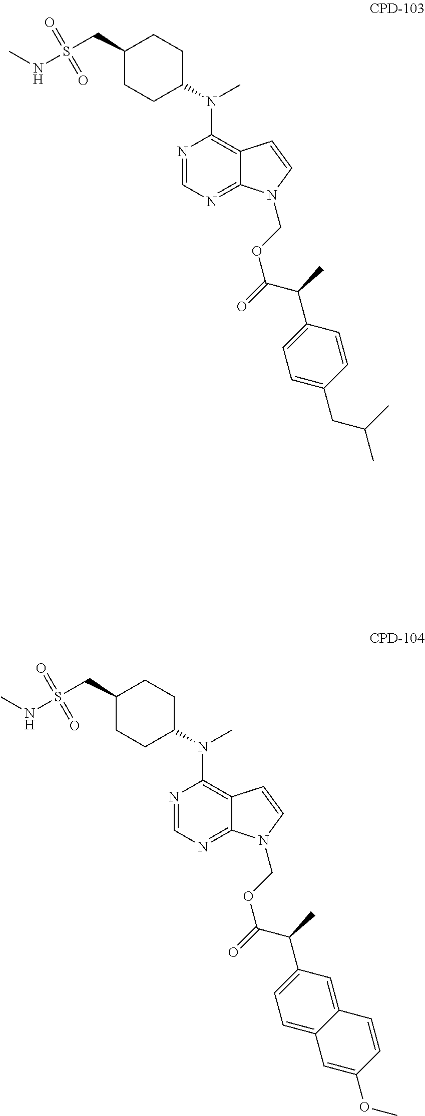

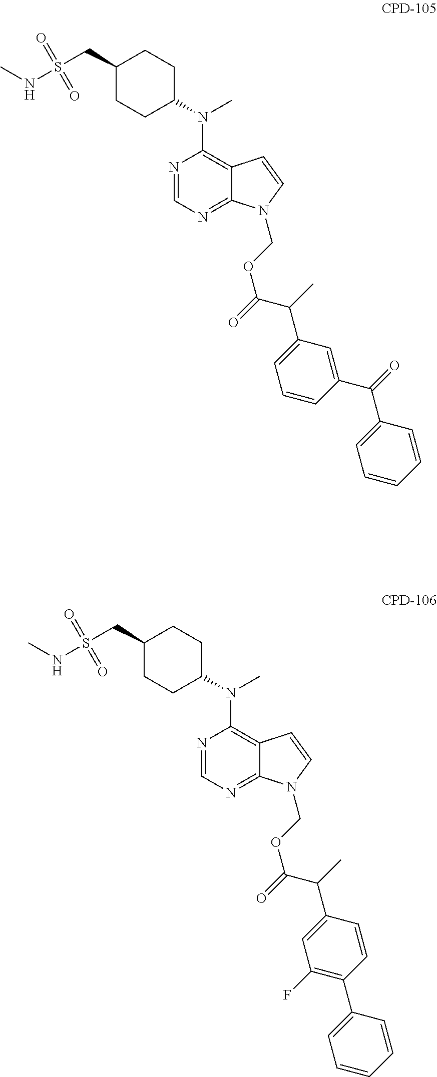

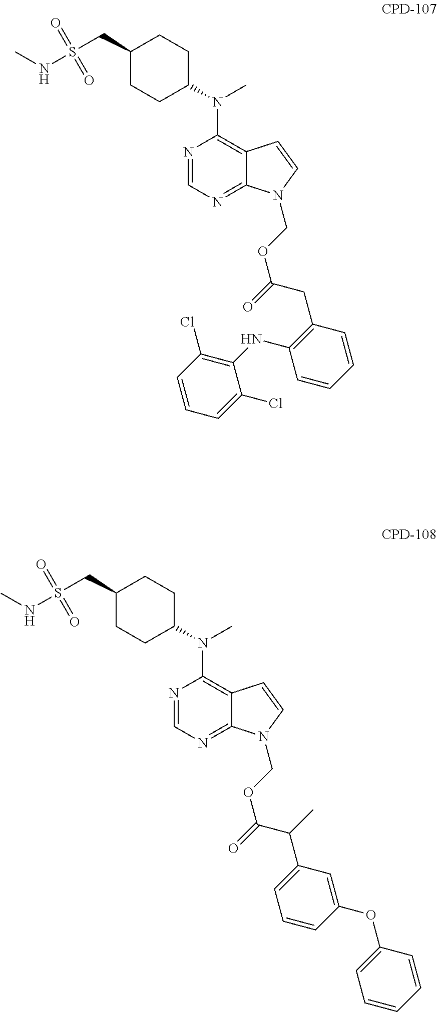

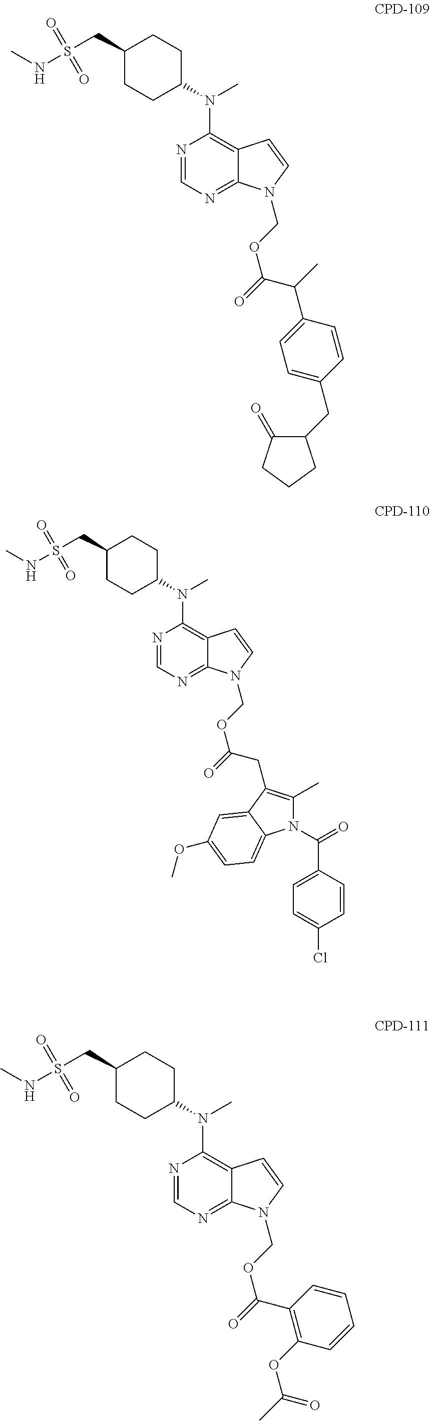

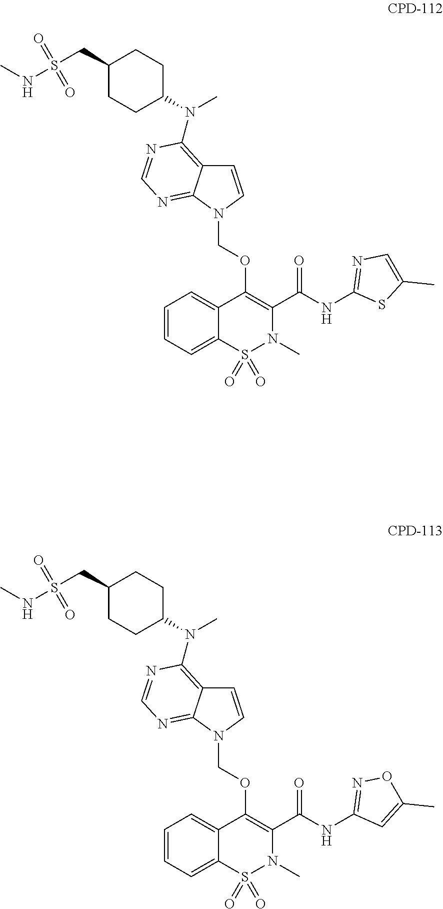

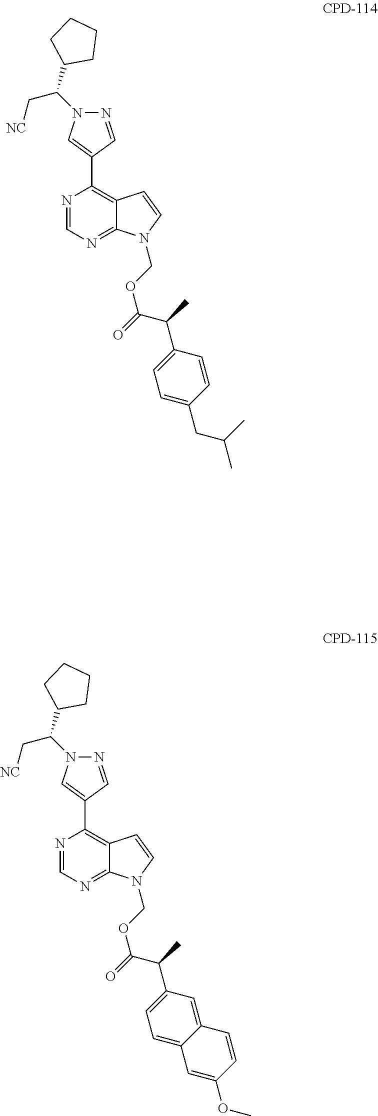 [SMILES: CC(C)Cc1ccc([C@H](C)C(=O)OCn2ccc3c(-c4cnn([C@H](CC#N)C5CCCC5)c4)ncnc32)cc1 ; COc1ccc2cc([C@H](C)C(=O)OCn3ccc4c(-c5cnn([C@H](CC#N)C6CCCC6)c5)ncnc43)ccc2c1]

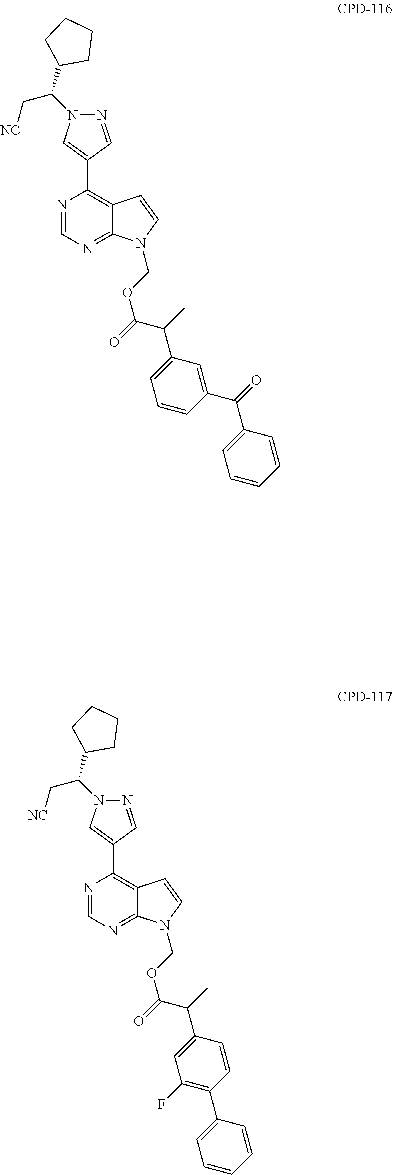 [SMILES: CC(C(=O)OCn1ccc2c(-c3cnn([C@H](CC#N)C4CCCC4)c3)ncnc21)c1cccc(C(=O)c2ccccc2)c1 ; CC(C(=O)OCn1ccc2c(-c3cnn([C@H](CC#N)C4CCCC4)c3)ncnc21)c1ccc(-c2ccccc2)c(F)c1]

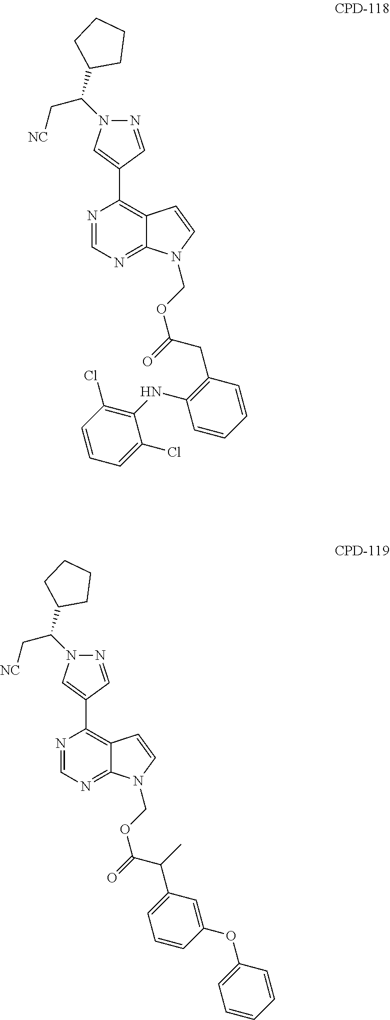 [SMILES: N#CC[C@H](C1CCCC1)n1cc(-c2ncnc3c2ccn3COC(=O)Cc2ccccc2Nc2c(Cl)cccc2Cl)cn1 ; CC(C(=O)OCn1ccc2c(-c3cnn([C@H](CC#N)C4CCCC4)c3)ncnc21)c1cccc(Oc2ccccc2)c1]

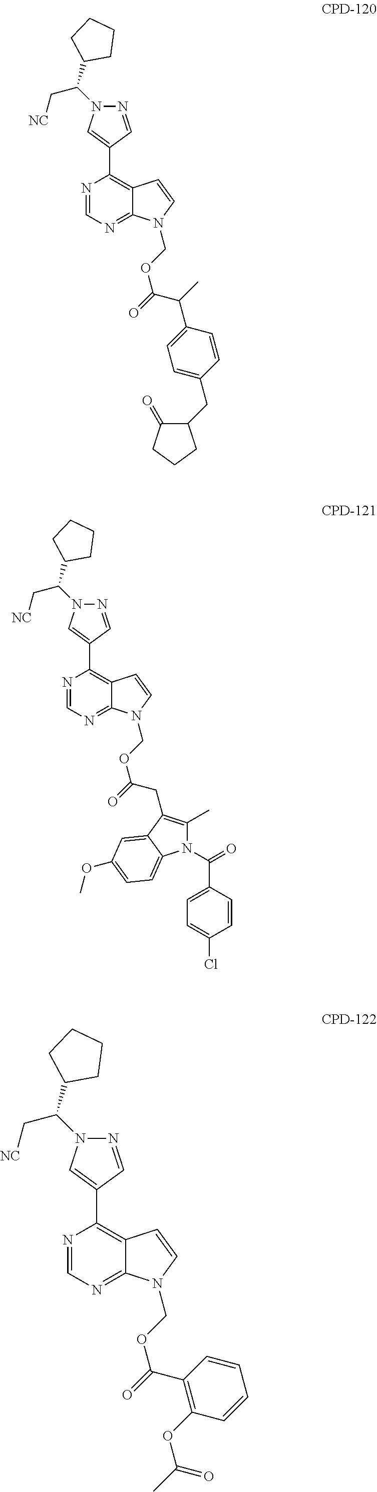 [SMILES: CC(C(=O)OCn1ccc2c(-c3cnn([C@H](CC#N)C4CCCC4)c3)ncnc21)c1ccc(CC2CCCC2=O)cc1 ; COc1ccc2c(c1)c(CC(=O)OCn1ccc3c(-c4cnn([C@H](CC#N)C5CCCC5)c4)ncnc31)c(C)n2C(=O)c1ccc(Cl)cc1 ; CC(=O)Oc1ccccc1C(=O)OCn1ccc2c(-c3cnn([C@H](CC#N)C4CCCC4)c3)ncnc21]

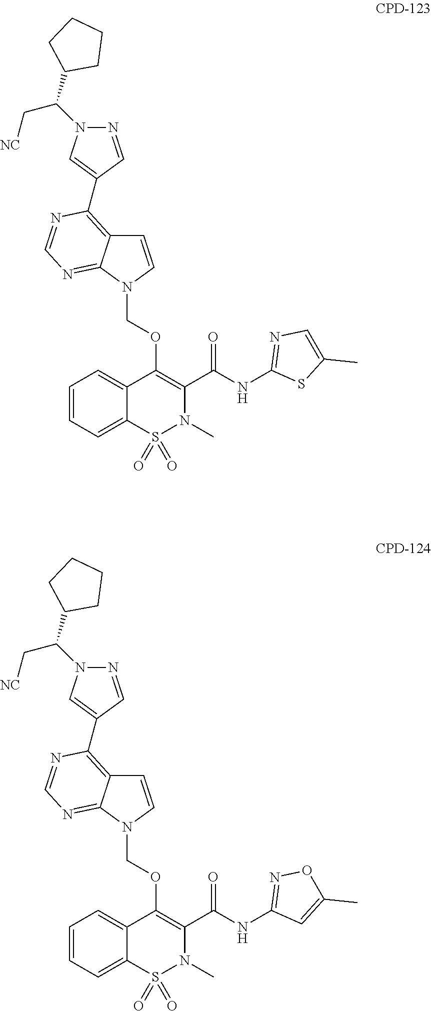 [SMILES: Cc1cnc(NC(=O)C2=C(OCn3ccc4c(-c5cnn([C@H](CC#N)C6CCCC6)c5)ncnc43)c3ccccc3S(=O)(=O)N2C)s1 ; Cc1cc(NC(=O)C2=C(OCn3ccc4c(-c5cnn([C@H](CC#N)C6CCCC6)c5)ncnc43)c3ccccc3S(=O)(=O)N2C)no1]

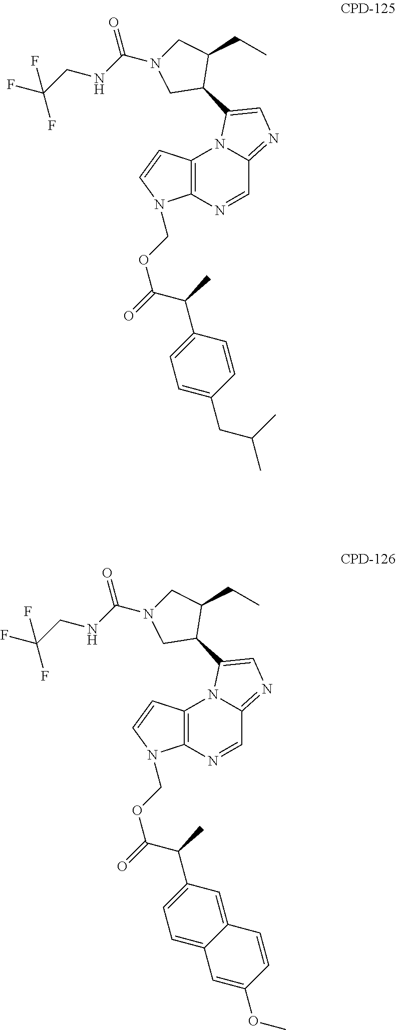 [SMILES: CC[C@@H]1CN(C(=O)NCC(F)(F)F)C[C@@H]1c1cnc2cnc3c(ccn3COC(=O)[C@@H](C)c3ccc(CC(C)C)cc3)n12 ; CC[C@@H]1CN(C(=O)NCC(F)(F)F)C[C@@H]1c1cnc2cnc3c(ccn3COC(=O)[C@@H](C)c3ccc4cc(OC)ccc4c3)n12]

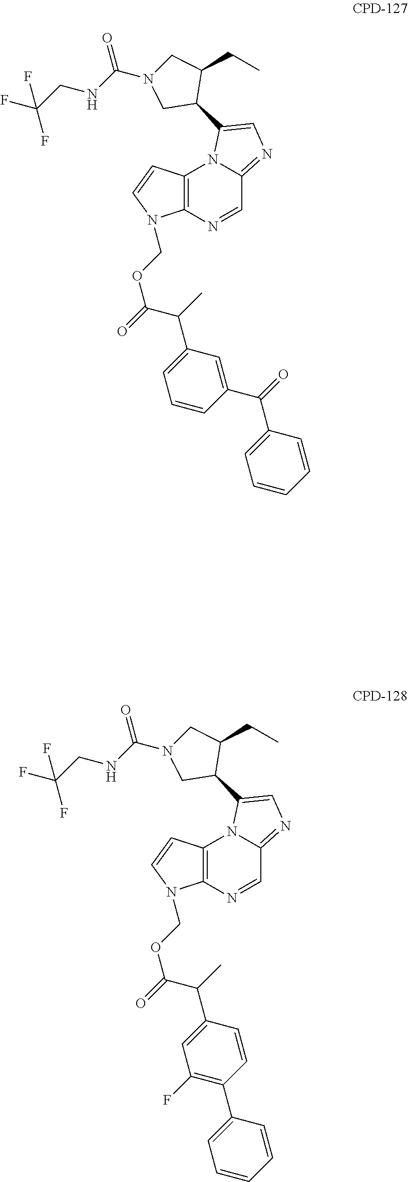 [SMILES: CC[C@@H]1CN(C(=O)NCC(F)(F)F)C[C@@H]1c1cnc2cnc3c(ccn3COC(=O)C(C)c3cccc(C(=O)c4ccccc4)c3)n12 ; CC[C@@H]1CN(C(=O)NCC(F)(F)F)C[C@@H]1c1cnc2cnc3c(ccn3COC(=O)C(C)c3ccc(-c4ccccc4)c(F)c3)n12]

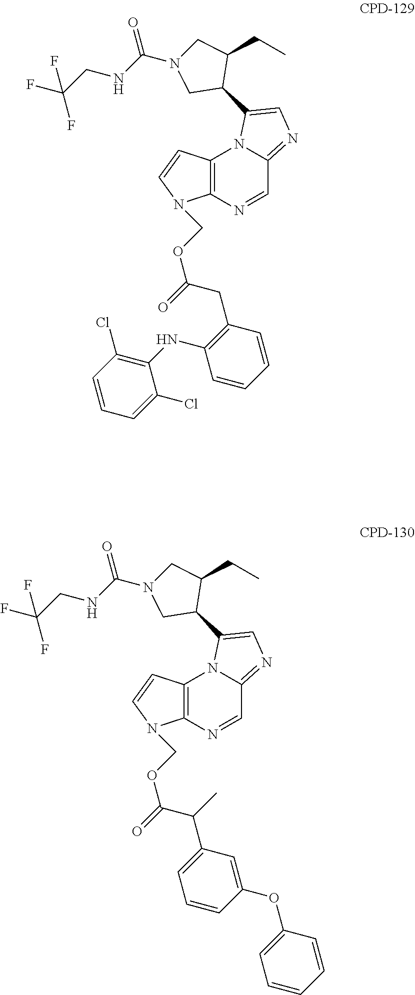 [SMILES: CC[C@@H]1CN(C(=O)NCC(F)(F)F)C[C@@H]1c1cnc2cnc3c(ccn3COC(=O)Cc3ccccc3Nc3c(Cl)cccc3Cl)n12 ; CC[C@@H]1CN(C(=O)NCC(F)(F)F)C[C@@H]1c1cnc2cnc3c(ccn3COC(=O)C(C)c3cccc(Oc4ccccc4)c3)n12]

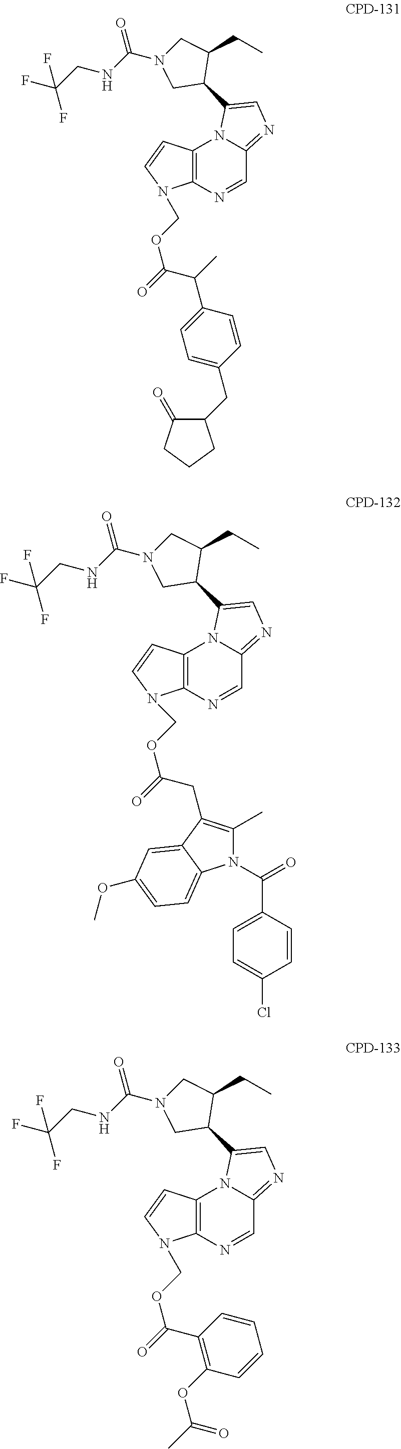 [SMILES: CC[C@@H]1CN(C(=O)NCC(F)(F)F)C[C@@H]1c1cnc2cnc3c(ccn3COC(=O)C(C)c3ccc(CC4CCCC4=O)cc3)n12 ; CC[C@@H]1CN(C(=O)NCC(F)(F)F)C[C@@H]1c1cnc2cnc3c(ccn3COC(=O)Cc3c(C)n(C(=O)c4ccc(Cl)cc4)c4ccc(OC)cc34)n12 ; CC[C@@H]1CN(C(=O)NCC(F)(F)F)C[C@@H]1c1cnc2cnc3c(ccn3COC(=O)c3ccccc3OC(C)=O)n12]

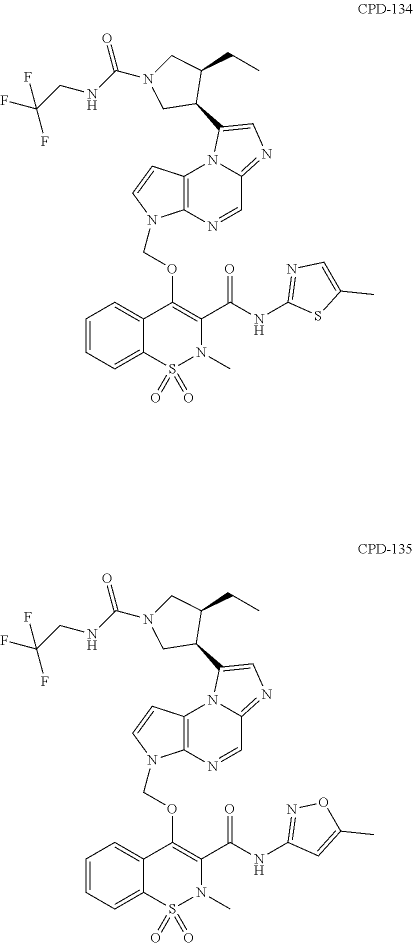 [SMILES: CC[C@@H]1CN(C(=O)NCC(F)(F)F)C[C@@H]1c1cnc2cnc3c(ccn3COC3=C(C(=O)Nc4ncc(C)s4)N(C)S(=O)(=O)c4ccccc43)n12 ; CC[C@@H]1CN(C(=O)NCC(F)(F)F)C[C@@H]1c1cnc2cnc3c(ccn3COC3=C(C(=O)Nc4cc(C)on4)N(C)S(=O)(=O)c4ccccc43)n12]

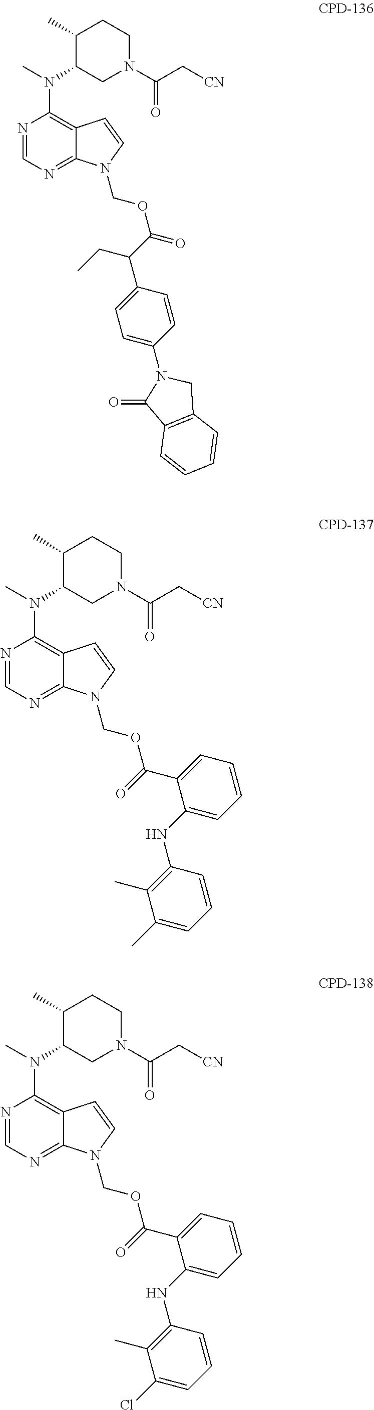 [SMILES: CCC(C(=O)OCn1ccc2c(N(C)[C@H]3CN(C(=O)CC#N)CC[C@H]3C)ncnc21)c1ccc(N2Cc3ccccc3C2=O)cc1 ; Cc1cccc(Nc2ccccc2C(=O)OCn2ccc3c(N(C)[C@H]4CN(C(=O)CC#N)CC[C@H]4C)ncnc32)c1C ; Cc1c(Cl)cccc1Nc1ccccc1C(=O)OCn1ccc2c(N(C)[C@H]3CN(C(=O)CC#N)CC[C@H]3C)ncnc21]

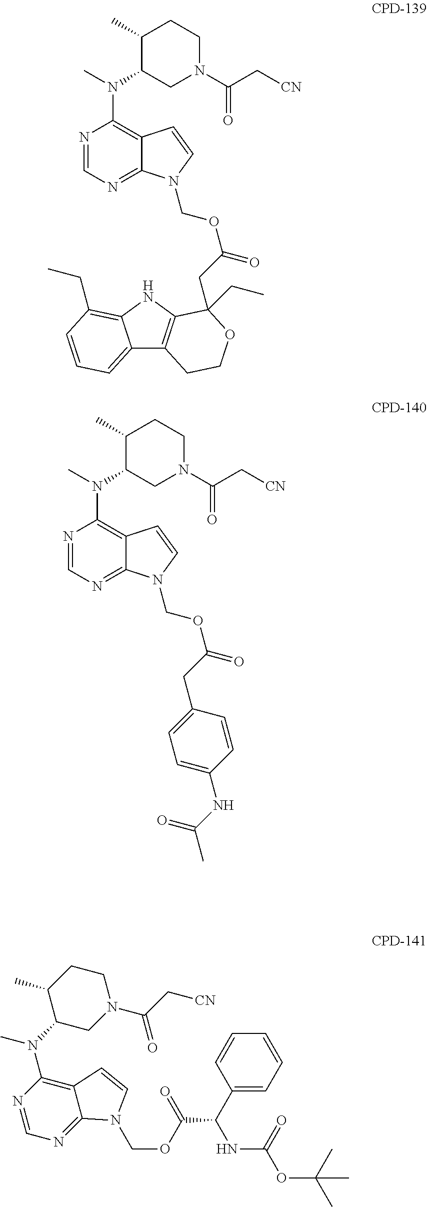 [SMILES: CCc1cccc2c3c([nH]c12)C(CC)(CC(=O)OCn1ccc2c(N(C)[C@H]4CN(C(=O)CC#N)CC[C@H]4C)ncnc21)OCC3 ; CC(=O)Nc1ccc(CC(=O)OCn2ccc3c(N(C)[C@H]4CN(C(=O)CC#N)CC[C@H]4C)ncnc32)cc1 ; C[C@@H]1CCN(C(=O)CC#N)C[C@@H]1N(C)c1ncnc2c1ccn2COC(=O)[C@@H](NC(=O)OC(C)(C)C)c1ccccc1]

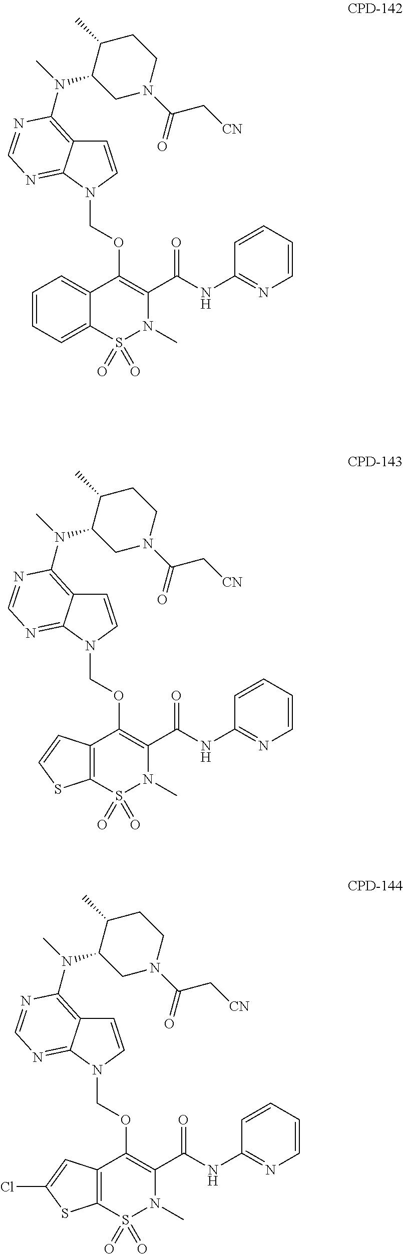 [SMILES: C[C@@H]1CCN(C(=O)CC#N)C[C@@H]1N(C)c1ncnc2c1ccn2COC1=C(C(=O)Nc2ccccn2)N(C)S(=O)(=O)c2ccccc21 ; C[C@@H]1CCN(C(=O)CC#N)C[C@@H]1N(C)c1ncnc2c1ccn2COC1=C(C(=O)Nc2ccccn2)N(C)S(=O)(=O)c2sccc21 ; C[C@@H]1CCN(C(=O)CC#N)C[C@@H]1N(C)c1ncnc2c1ccn2COC1=C(C(=O)Nc2ccccn2)N(C)S(=O)(=O)c2sc(Cl)cc21]

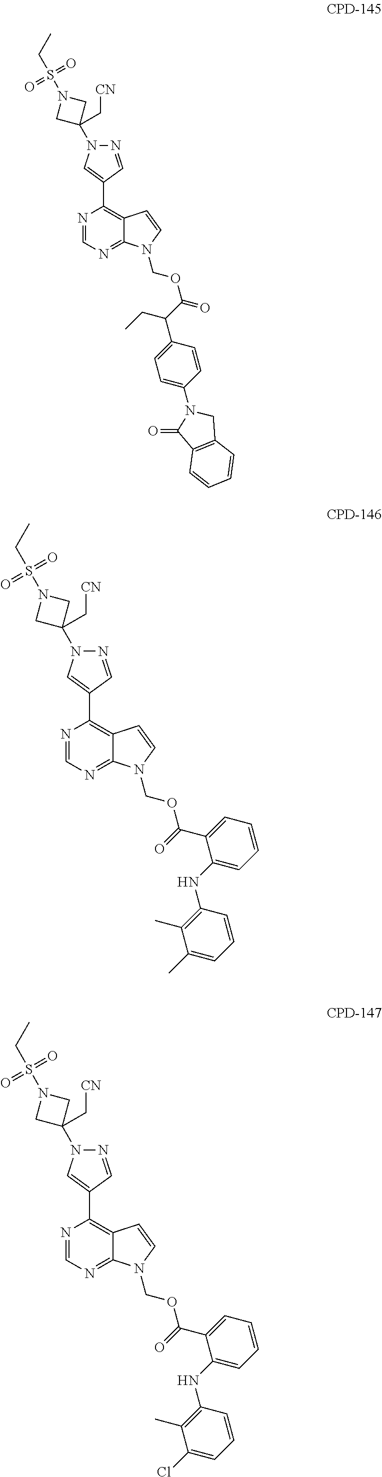 [SMILES: CCC(C(=O)OCn1ccc2c(-c3cnn(C4(CC#N)CN(S(=O)(=O)CC)C4)c3)ncnc21)c1ccc(N2Cc3ccccc3C2=O)cc1 ; CCS(=O)(=O)N1CC(CC#N)(n2cc(-c3ncnc4c3ccn4COC(=O)c3ccccc3Nc3cccc(C)c3C)cn2)C1 ; CCS(=O)(=O)N1CC(CC#N)(n2cc(-c3ncnc4c3ccn4COC(=O)c3ccccc3Nc3cccc(Cl)c3C)cn2)C1]

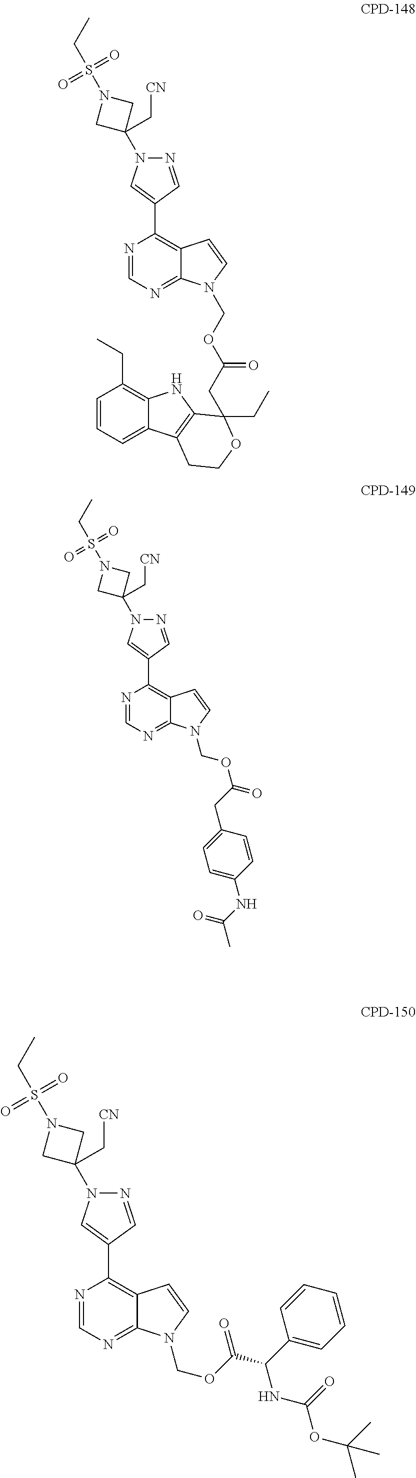 [SMILES: CCc1cccc2c3c([nH]c12)C(CC)(CC(=O)OCn1ccc2c(-c4cnn(C5(CC#N)CN(S(=O)(=O)CC)C5)c4)ncnc21)OCC3 ; CCS(=O)(=O)N1CC(CC#N)(n2cc(-c3ncnc4c3ccn4COC(=O)Cc3ccc(NC(C)=O)cc3)cn2)C1 ; CCS(=O)(=O)N1CC(CC#N)(n2cc(-c3ncnc4c3ccn4COC(=O)[C@@H](NC(=O)OC(C)(C)C)c3ccccc3)cn2)C1]

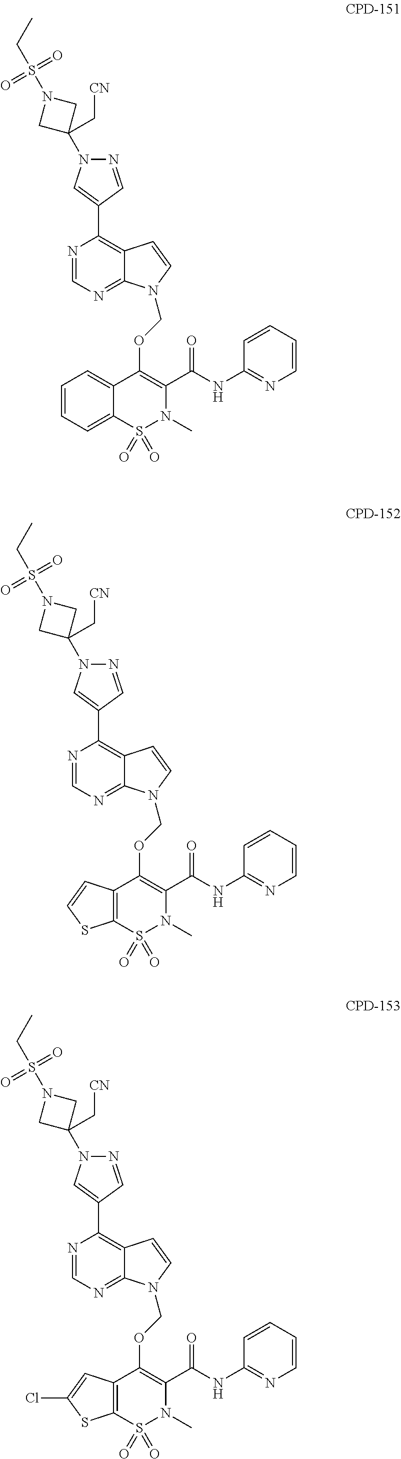 [SMILES: CCS(=O)(=O)N1CC(CC#N)(n2cc(-c3ncnc4c3ccn4COC3=C(C(=O)Nc4ccccn4)N(C)S(=O)(=O)c4ccccc43)cn2)C1 ; CCS(=O)(=O)N1CC(CC#N)(n2cc(-c3ncnc4c3ccn4COC3=C(C(=O)Nc4ccccn4)N(C)S(=O)(=O)c4sccc43)cn2)C1 ; CCS(=O)(=O)N1CC(CC#N)(n2cc(-c3ncnc4c3ccn4COC3=C(C(=O)Nc4ccccn4)N(C)S(=O)(=O)c4sc(Cl)cc43)cn2)C1]

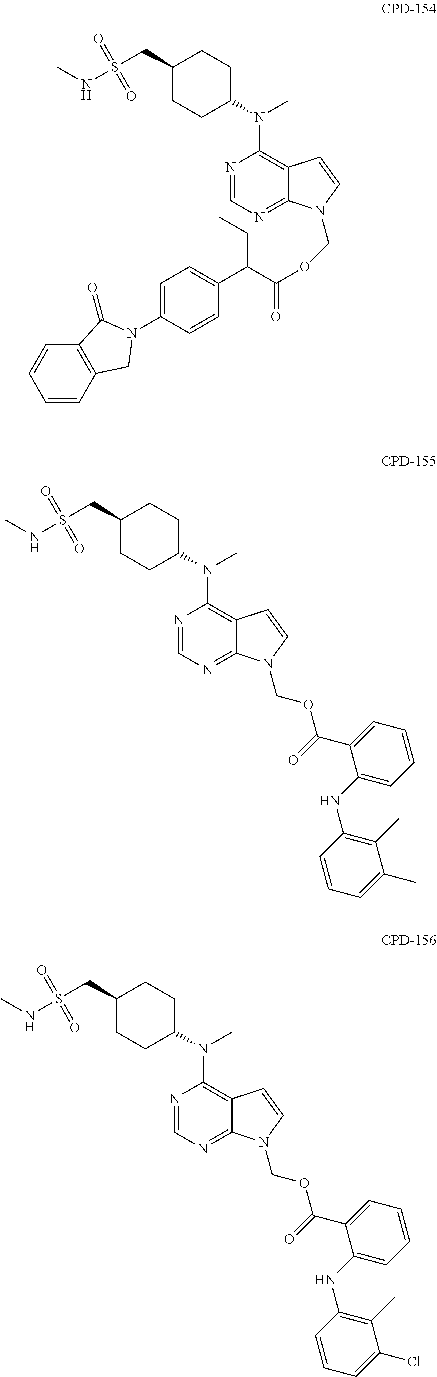

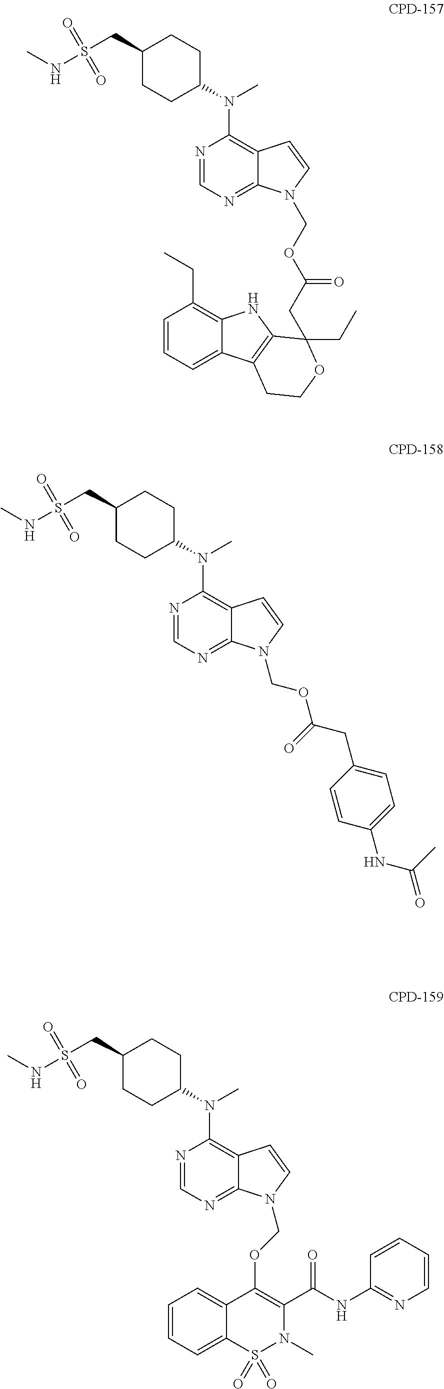

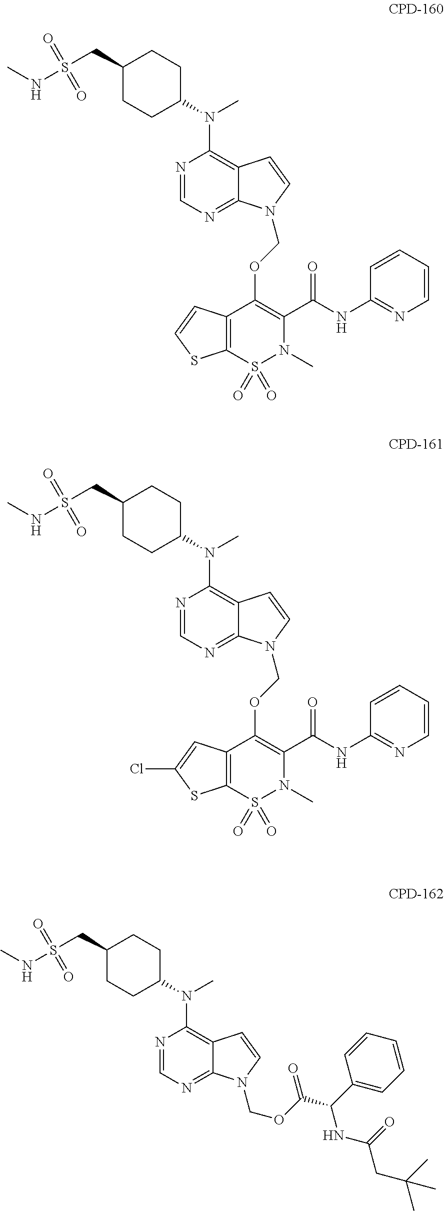

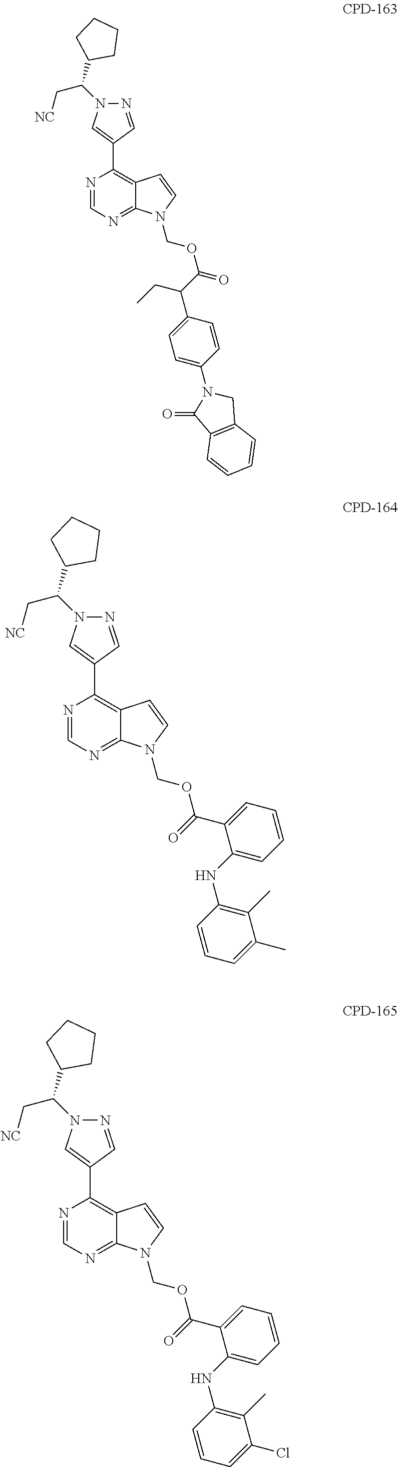 [SMILES: CCC(C(=O)OCn1ccc2c(-c3cnn([C@H](CC#N)C4CCCC4)c3)ncnc21)c1ccc(N2Cc3ccccc3C2=O)cc1 ; Cc1cccc(Nc2ccccc2C(=O)OCn2ccc3c(-c4cnn([C@H](CC#N)C5CCCC5)c4)ncnc32)c1C ; Cc1c(Cl)cccc1Nc1ccccc1C(=O)OCn1ccc2c(-c3cnn([C@H](CC#N)C4CCCC4)c3)ncnc21]

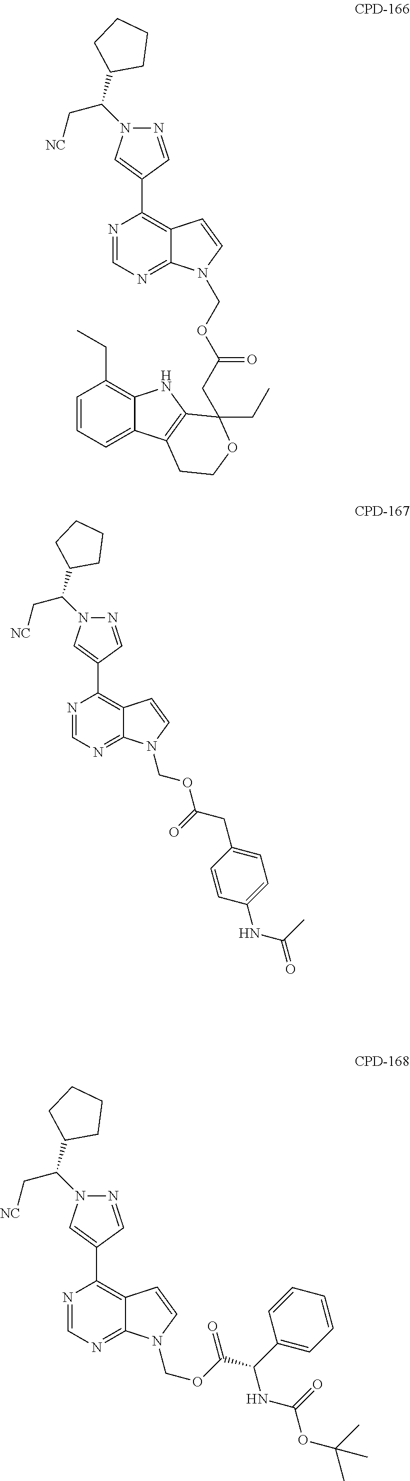 [SMILES: CCc1cccc2c3c([nH]c12)C(CC)(CC(=O)OCn1ccc2c(-c4cnn([C@H](CC#N)C5CCCC5)c4)ncnc21)OCC3 ; CC(=O)Nc1ccc(CC(=O)OCn2ccc3c(-c4cnn([C@H](CC#N)C5CCCC5)c4)ncnc32)cc1 ; CC(C)(C)OC(=O)N[C@H](C(=O)OCn1ccc2c(-c3cnn([C@H](CC#N)C4CCCC4)c3)ncnc21)c1ccccc1]

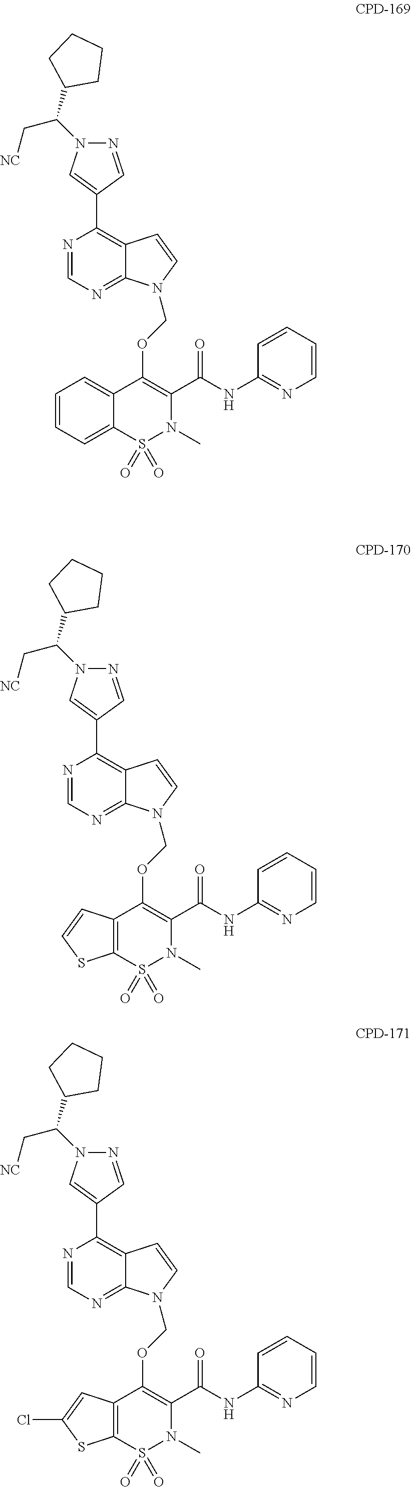 [SMILES: CN1C(C(=O)Nc2ccccn2)=C(OCn2ccc3c(-c4cnn([C@H](CC#N)C5CCCC5)c4)ncnc32)c2ccccc2S1(=O)=O ; CN1C(C(=O)Nc2ccccn2)=C(OCn2ccc3c(-c4cnn([C@H](CC#N)C5CCCC5)c4)ncnc32)c2ccsc2S1(=O)=O ; CN1C(C(=O)Nc2ccccn2)=C(OCn2ccc3c(-c4cnn([C@H](CC#N)C5CCCC5)c4)ncnc32)c2cc(Cl)sc2S1(=O)=O]

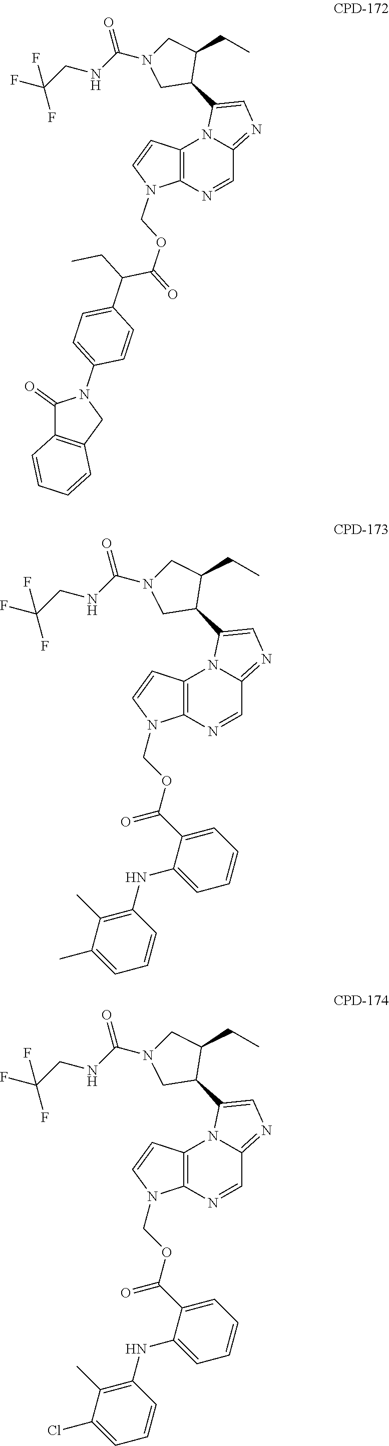

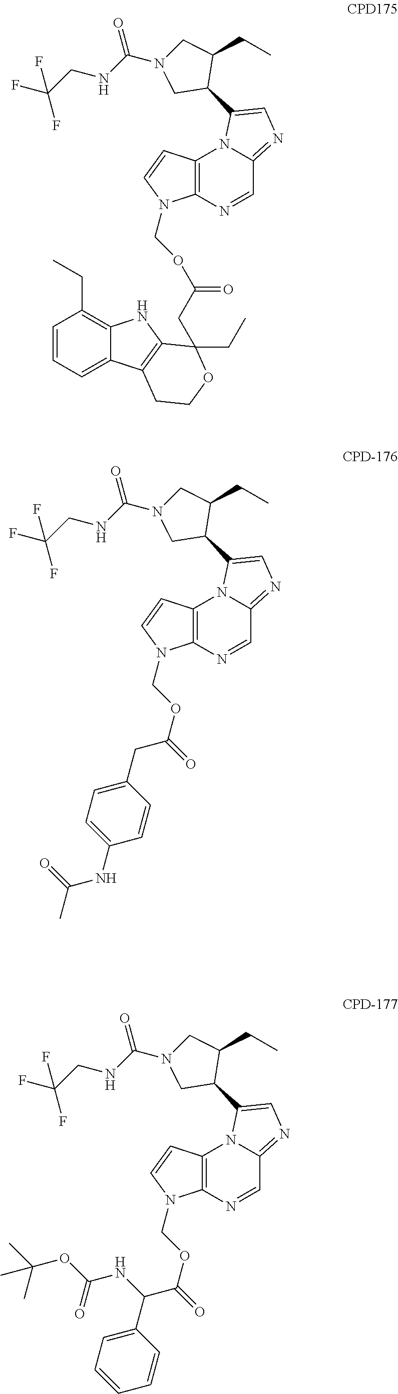 [SMILES: CCc1cccc2c3c([nH]c12)C(CC)(CC(=O)OCn1ccc2c1ncc1ncc([C@H]4CN(C(=O)NCC(F)(F)F)C[C@H]4CC)n12)OCC3 ; CC[C@@H]1CN(C(=O)NCC(F)(F)F)C[C@@H]1c1cnc2cnc3c(ccn3COC(=O)Cc3ccc(NC(C)=O)cc3)n12 ; CC[C@@H]1CN(C(=O)NCC(F)(F)F)C[C@@H]1c1cnc2cnc3c(ccn3COC(=O)C(NC(=O)OC(C)(C)C)c3ccccc3)n12]

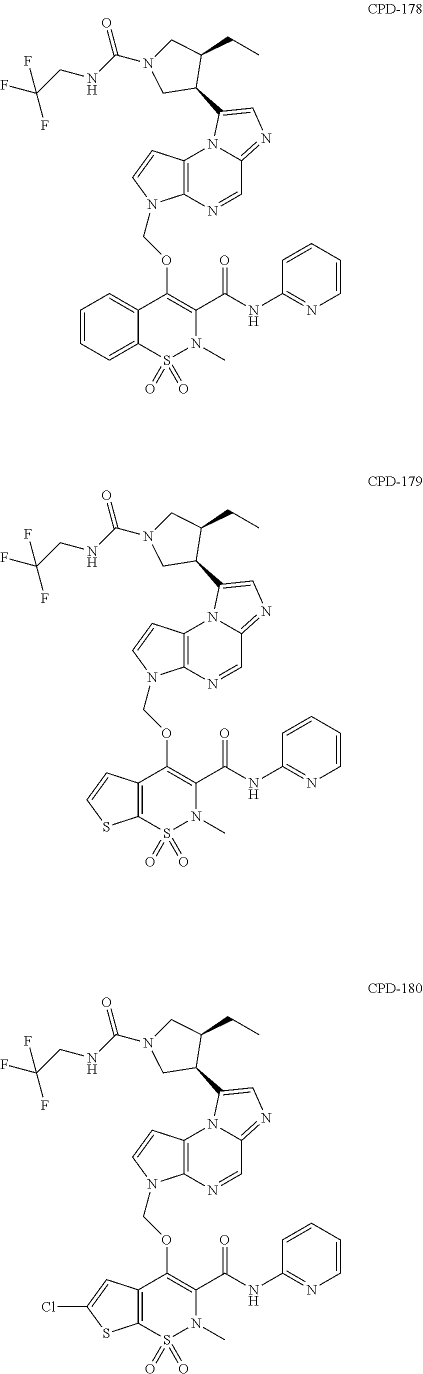 [SMILES: CC[C@@H]1CN(C(=O)NCC(F)(F)F)C[C@@H]1c1cnc2cnc3c(ccn3COC3=C(C(=O)Nc4ccccn4)N(C)S(=O)(=O)c4ccccc43)n12 ; CC[C@@H]1CN(C(=O)NCC(F)(F)F)C[C@@H]1c1cnc2cnc3c(ccn3COC3=C(C(=O)Nc4ccccn4)N(C)S(=O)(=O)c4sccc43)n12 ; CC[C@@H]1CN(C(=O)NCC(F)(F)F)C[C@@H]1c1cnc2cnc3c(ccn3COC3=C(C(=O)Nc4ccccn4)N(C)S(=O)(=O)c4sc(Cl)cc43)n12]

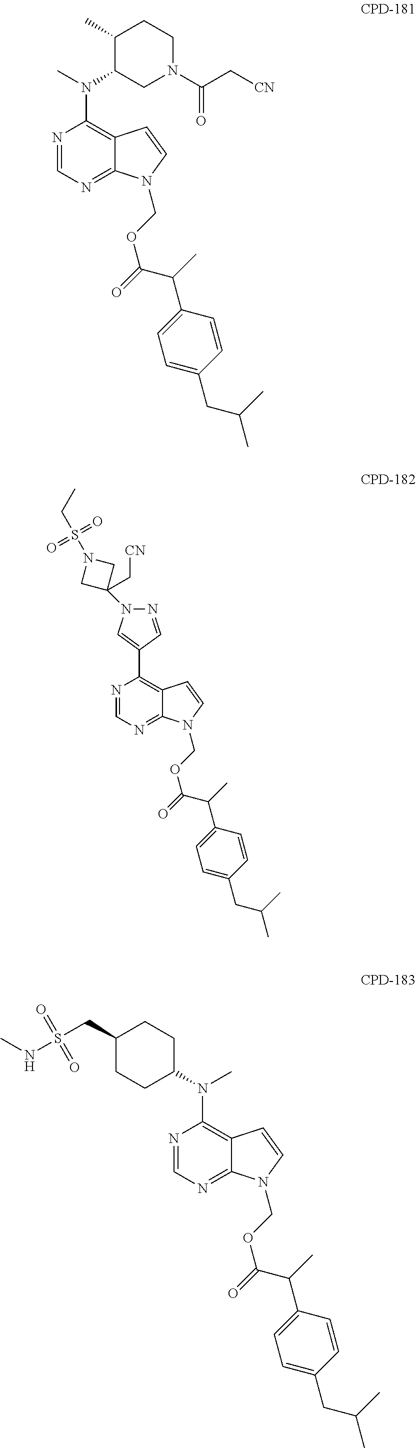

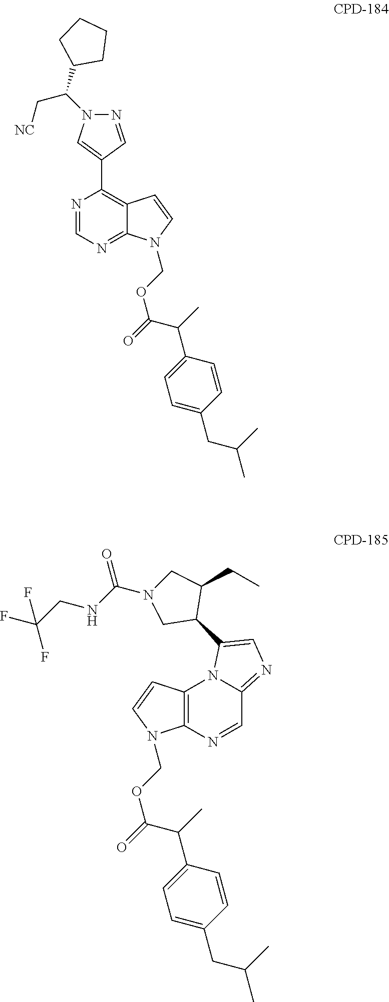 [SMILES: CC(C)Cc1ccc(C(C)C(=O)OCn2ccc3c(-c4cnn([C@H](CC#N)C5CCCC5)c4)ncnc32)cc1 ; CC[C@@H]1CN(C(=O)NCC(F)(F)F)C[C@@H]1c1cnc2cnc3c(ccn3COC(=O)C(C)c3ccc(CC(C)C)cc3)n12]

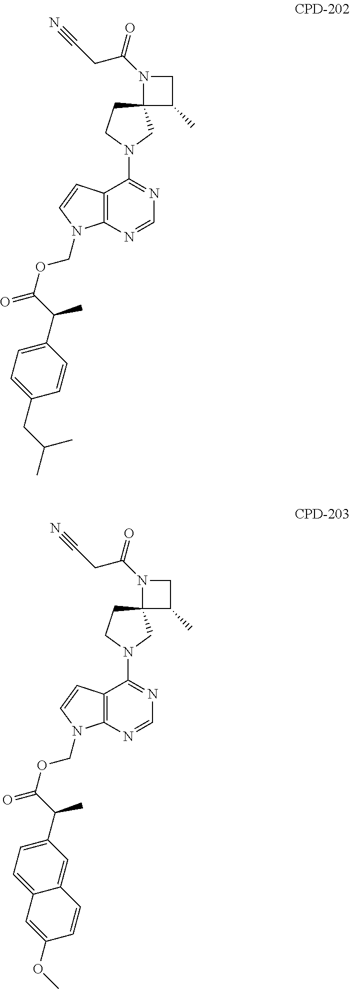 [SMILES: CC(C)Cc1ccc([C@H](C)C(=O)OCn2ccc3c(N4CC[C@]5(C4)[C@@H](C)CN5C(=O)CC#N)ncnc32)cc1 ; COc1ccc2cc([C@H](C)C(=O)OCn3ccc4c(N5CC[C@]6(C5)[C@@H](C)CN6C(=O)CC#N)ncnc43)ccc2c1]

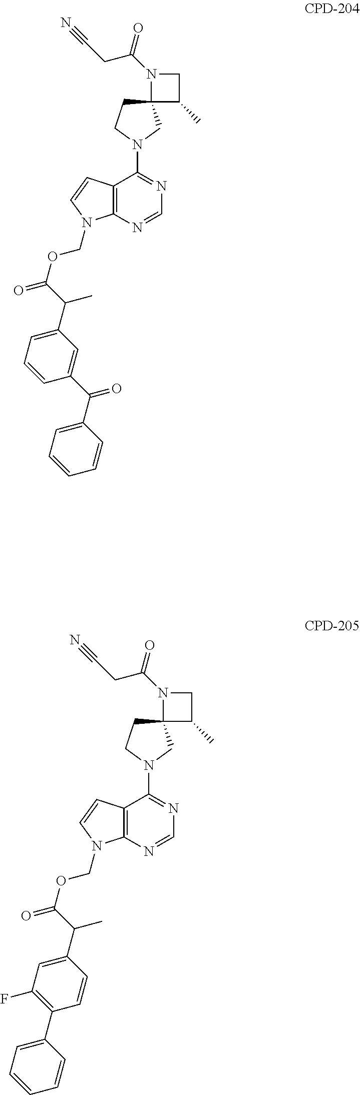 [SMILES: CC(C(=O)OCn1ccc2c(N3CC[C@]4(C3)[C@@H](C)CN4C(=O)CC#N)ncnc21)c1cccc(C(=O)c2ccccc2)c1 ; CC(C(=O)OCn1ccc2c(N3CC[C@]4(C3)[C@@H](C)CN4C(=O)CC#N)ncnc21)c1ccc(-c2ccccc2)c(F)c1]

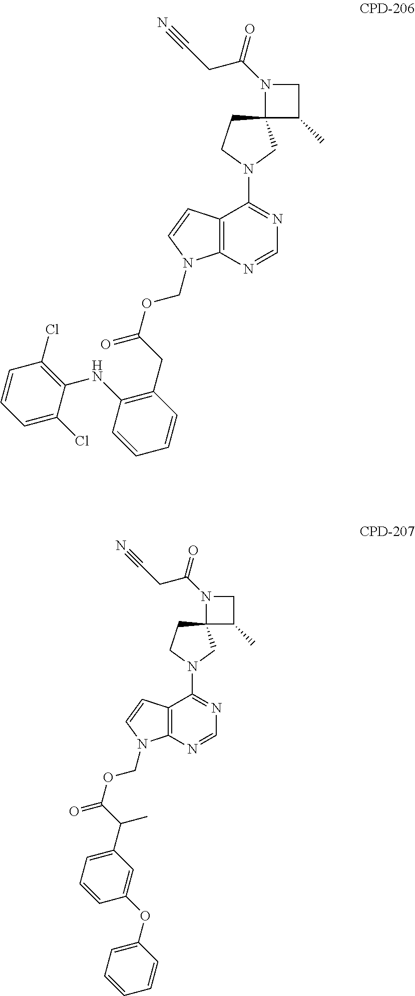 [SMILES: C[C@H]1CN(C(=O)CC#N)[C@]12CCN(c1ncnc3c1ccn3COC(=O)Cc1ccccc1Nc1c(Cl)cccc1Cl)C2 ; CC(C(=O)OCn1ccc2c(N3CC[C@]4(C3)[C@@H](C)CN4C(=O)CC#N)ncnc21)c1cccc(Oc2ccccc2)c1]

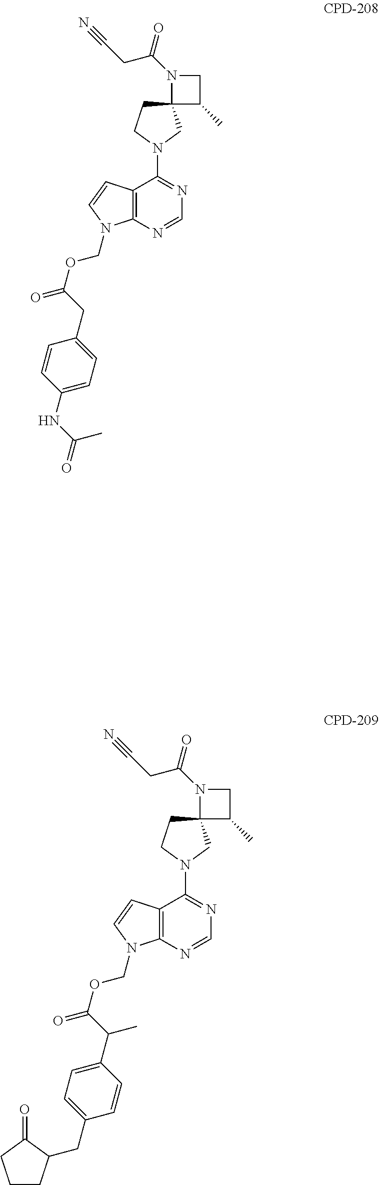 [SMILES: CC(=O)Nc1ccc(CC(=O)OCn2ccc3c(N4CC[C@]5(C4)[C@@H](C)CN5C(=O)CC#N)ncnc32)cc1 ; CC(C(=O)OCn1ccc2c(N3CC[C@]4(C3)[C@@H](C)CN4C(=O)CC#N)ncnc21)c1ccc(CC2CCCC2=O)cc1]

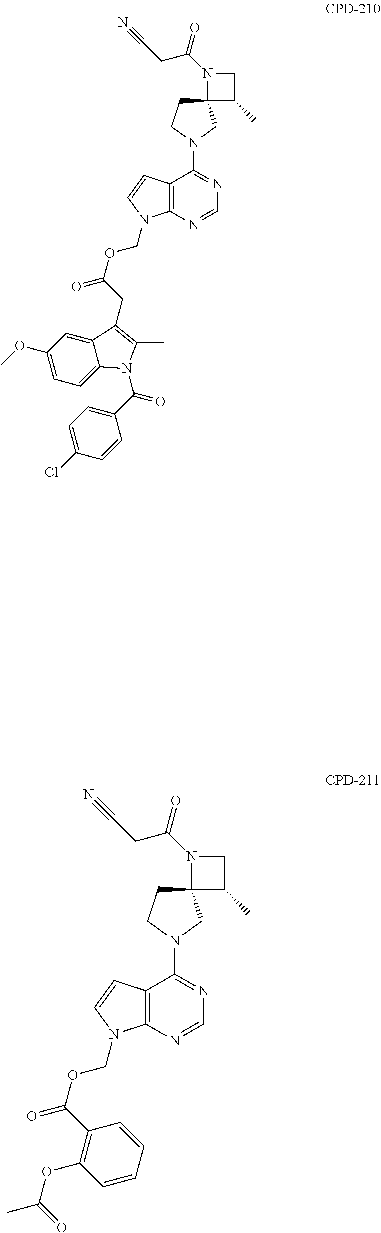

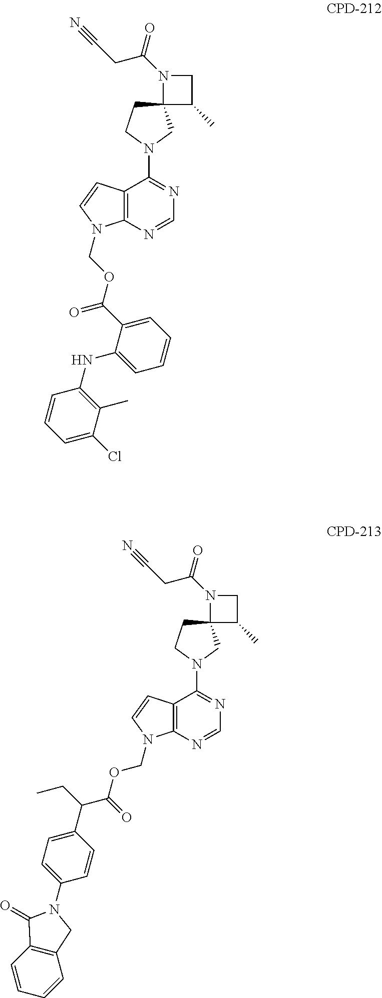 [SMILES: Cc1c(Cl)cccc1Nc1ccccc1C(=O)OCn1ccc2c(N3CC[C@]4(C3)[C@@H](C)CN4C(=O)CC#N)ncnc21 ; CCC(C(=O)OCn1ccc2c(N3CC[C@]4(C3)[C@@H](C)CN4C(=O)CC#N)ncnc21)c1ccc(N2Cc3ccccc3C2=O)cc1]

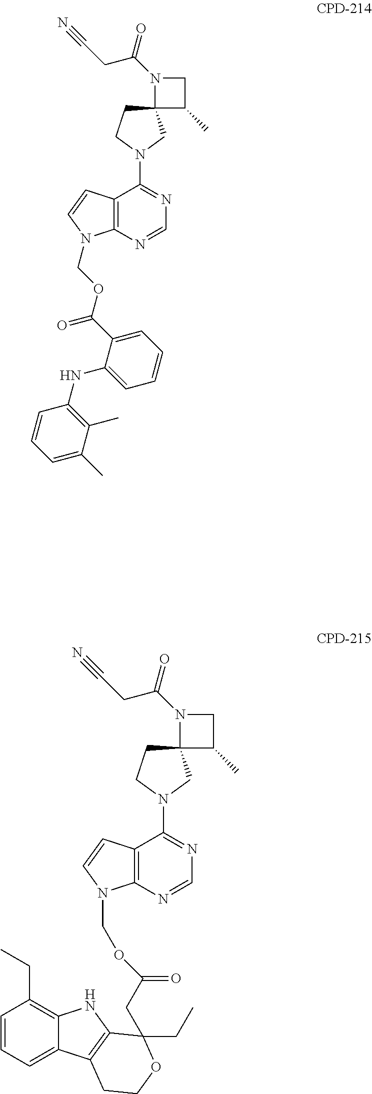 [SMILES: Cc1cccc(Nc2ccccc2C(=O)OCn2ccc3c(N4CC[C@]5(C4)[C@@H](C)CN5C(=O)CC#N)ncnc32)c1C ; CCc1cccc2c3c([nH]c12)C(CC)(CC(=O)OCn1ccc2c(N4CC[C@]5(C4)[C@@H](C)CN5C(=O)CC#N)ncnc21)OCC3]

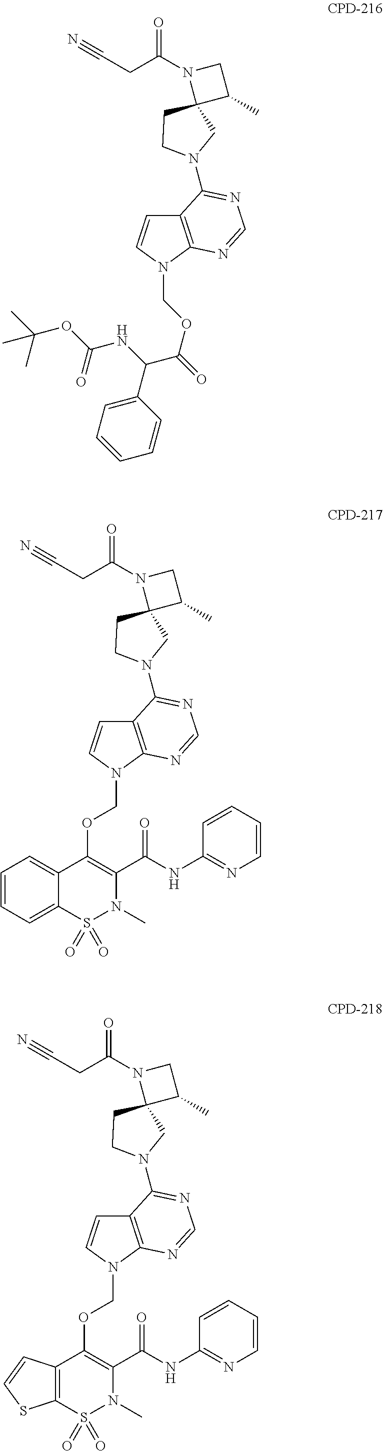 [SMILES: C[C@H]1CN(C(=O)CC#N)[C@]12CCN(c1ncnc3c1ccn3COC(=O)C(NC(=O)OC(C)(C)C)c1ccccc1)C2 ; C[C@H]1CN(C(=O)CC#N)[C@]12CCN(c1ncnc3c1ccn3COC1=C(C(=O)Nc3ccccn3)N(C)S(=O)(=O)c3ccccc31)C2 ; C[C@H]1CN(C(=O)CC#N)[C@]12CCN(c1ncnc3c1ccn3COC1=C(C(=O)Nc3ccccn3)N(C)S(=O)(=O)c3sccc31)C2]

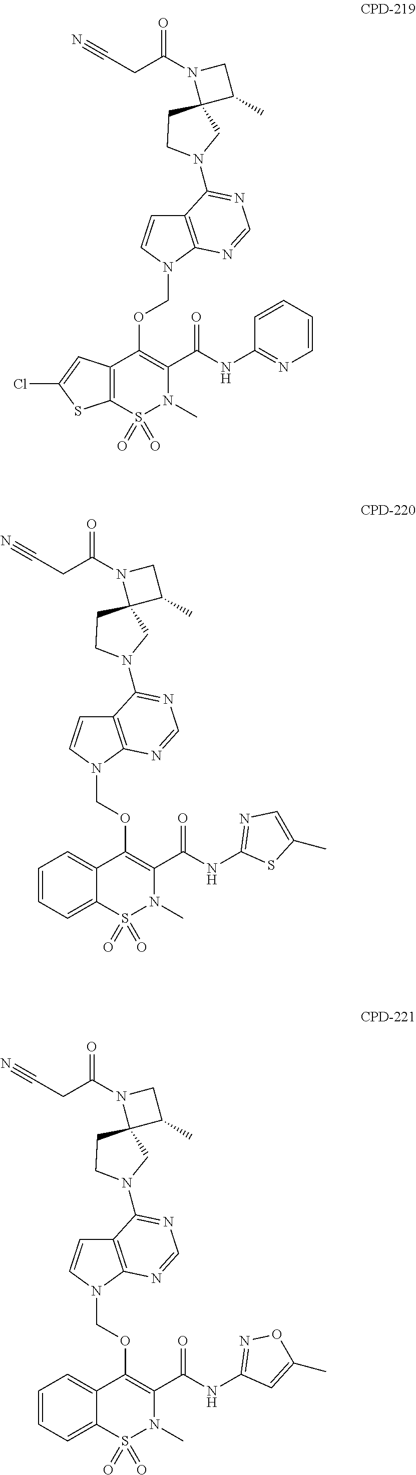 [SMILES: C[C@H]1CN(C(=O)CC#N)[C@]12CCN(c1ncnc3c1ccn3COC1=C(C(=O)Nc3ccccn3)N(C)S(=O)(=O)c3sc(Cl)cc31)C2 ; Cc1cnc(NC(=O)C2=C(OCn3ccc4c(N5CC[C@]6(C5)[C@@H](C)CN6C(=O)CC#N)ncnc43)c3ccccc3S(=O)(=O)N2C)s1 ; Cc1cc(NC(=O)C2=C(OCn3ccc4c(N5CC[C@]6(C5)[C@@H](C)CN6C(=O)CC#N)ncnc43)c3ccccc3S(=O)(=O)N2C)no1]

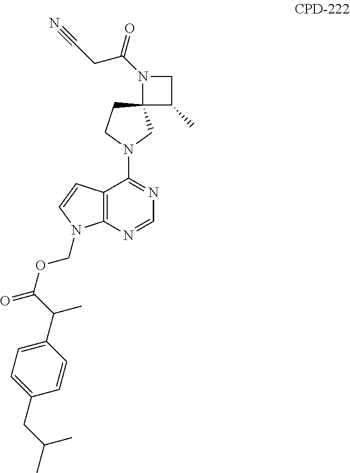 [SMILES: CC(C)Cc1ccc(C(C)C(=O)OCn2ccc3c(N4CC[C@]5(C4)[C@@H](C)CN5C(=O)CC#N)ncnc32)cc1]

7. A method of treating an anti-inflammatory symptom, comprising administering the anti-inflammatory compound, or the stereoisomer, tautomer, nitrogen oxide, prodrug, pharmaceutically acceptable salt, or solvate thereof of claim 1.

8. The anti-inflammatory compound, or the stereoisomer, tautomer, nitrogen oxide, prodrug, pharmaceutically acceptable salt, or solvate thereof of claim 12, wherein the substituted cyclohexyl group is a cyclohexyl group substituted with an amino group and a sulfonyl group, and the substituted piperidinyl group is a piperidinyl group substituted with an acyl group or a sulfonyl group and —CN.

9. The anti-inflammatory compound, or the stereoisomer, tautomer, nitrogen oxide, prodrug, pharmaceutically acceptable salt, or solvate thereof of claim 3, wherein Ra and Rb are each independently groups which consist of one group of acyl or sulfonyl and at least one group selected from a group consisting of cyano, amino or C1-C6 alkyl substituted amino and a four-, five-, or six-membered nitrogen-containing heterocyclyl, or the nitrogen-containing heterocyclyl substituted with C1-C6 alkyl, wherein the C1-C6 alkyl is substitutable by halogen.

10. The anti-inflammatory compound, or the stereoisomer, tautomer, nitrogen oxide, prodrug, pharmaceutically acceptable salt, or solvate thereof of claim 3, R_{d} is a thiazole or isoxazole substituted with methyl; or unsubstituted pyridyl.

11. The anti-inflammatory compound, or the stereoisomer, tautomer, nitrogen oxide, prodrug, pharmaceutically acceptable salt, or solvate thereof of claim 3, Ar is an aromatic ring group selected from a benzene ring, a naphthalene ring or an aryl heterocyclic ring; and a benzene ring, a naphthalene ring, or an aryl heterocyclic ring or an aryl fused heterocyclic ring substituted with one or more groups selected from halogen, a C₁-C₆ alkyl group, a C₁-C₆ alkoxy group, a C₁-C₆ acyl group, or a C₁-C₆ alkoxy group.

12. The anti-inflammatory compound, or the stereoisomer, tautomer, nitrogen oxide, prodrug, pharmaceutically acceptable salt, or solvate thereof of claim 3, wherein Cy is substituted cyclohexyl or substituted piperidinyl.

13. The anti-inflammatory compound, or the stereoisomer, tautomer, nitrogen oxide, prodrug, pharmaceutically acceptable salt, or solvate thereof of claim 3, wherein the phenyl ring may be substituted by halogen or C₁-C₆ alkyl in the structural formula (a).
